(19) United States
(12) Patent Application Publication
(10) Pub. No.: US 2023/0052147 A1
(43) Pub. Date: Feb. 16, 2023

(54) IMAGE DIFFERENTIATED MULTIPLEX ASSAYS FOR DETECTION OF DNA MUTATIONS IN LUNG CANCER

(71) Applicant: PLEXBIO CO., LTD., Taipei City (TW)

(72) Inventors: Dean TSAO, Hillsborough, CA (US); Chin-Shiou HUANG, Taipei City (TW); Shian Pin HU, Taipei City, Taiwan (TW); Fei-Yu CHANG, Taipei City (TW); Yi Ling HSIEH, Taipei City (TW)

(73) Assignee: PLEXBIO CO., LTD., Taipei City (TW)

(21) Appl. No.: 17/758,511

(22) PCT Filed: Jan. 7, 2021
(86) PCT No.: PCT/US2021/012541

§ 371 Date: Jul. 7, 2022

Related U.S. Application Data
(60) Provisional application No. 62/958,599, filed on Jan. 8, 2020.

(51) Int. Cl. C12Q 1/6827; C12Q 1/6886; C12Q 1/6834

(52) U.S. Cl. CPC C12Q 1/6827; C12Q 1/6886; C12Q 1/6834; C12Q 2600/106; C12Q 2600/112; C12Q 2600/156; C12Q 2600/16

(57) ABSTRACT

Provided herein are methods and kits for detecting the presence of DNA and/or RNA mutations associated with cancer (e.g., lung cancer). The methods and kits employ microcarriers, each with a probe specific for a DNA or RNA mutation and an identifier unique to the probe sequence. Upon isolation and amplification of nucleic acids from a sample, hybridization of amplified DNA with a probe, specific for a DNA or RNA mutation, that is coupled to a microcarrier indicates the presence of the mutation in the sample. Since each microcarrier can be identified through detection of the identifier, multiplex screening assays are provided. Representative genes that can be screened for mutations include, e.g., KRAS, NRAS, PIK3CA, BRAF, EGFR, AKT1, MEK1, and HER2 for DNA mutations and/or ALK, ROS, RET, NTRK1, and cMET for RNA mutations.

### CROSS REFERENCE TO RELATED APPLICATIONS

This application claims the priority benefit of U.S. Patent Application No. 62/958,599, filed Jan. 8, 2020, the contents of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 695502001340SEQLIST.TXT, date recorded: Jan. 5, 2021, size: 245 KB).

### FIELD

Provided herein are methods for multiplex detection of DNA and/or RNA mutations in a sample using microcarriers, as well as kits related thereto. The microcarriers are encoded with an identifier and include a probe for detection of a mutation of interest.

### BACKGROUND

Early detection is a critically important factor in reducing the number of deaths attributable to cancer, since growth and metastasis of more advanced tumors are associated with increased mortality. Most cases of lung cancer are sporadic, rather than hereditary, but several genes and even types of specific mutations are commonly seen in these sporadic cases. For example, mutations in the EGFR, KRAS, MET, LKB1, BRAF, PIK3CA, ALK, RET, and ROS1 genes have been implicated as highly relevant in lung cancer (see El-Telbany, A. and Ma, P. C. (2012) Genes Cancer 3:467-480. Overall survival rates for lung cancer patients are still relatively low, but early detection and early identification of patients for targeted therapies (e.g., tyrosine kinase inhibitors for certain mutations) can lead to improved response rates. Therefore, there is a need for comprehensive and rapid tests that examine multiple genes simultaneously with a high level of accuracy, rather than individual gene testing.

Immunological and molecular diagnostic assays play a critical role both in the research and clinical fields. Often it is necessary to perform assays for a panel of multiple targets to gain a meaningful or bird's-eye view of results to facilitate research or clinical decision-making. This is particularly true in the era of genomics and proteomics, where an abundance of genetic markers and/or biomarkers are thought to influence or be predictive of particular disease states. In theory, assays of multiple targets can be accomplished by testing each target separately in parallel or sequentially in different reaction vessels (i.e., multiple singleplexing). However, not only are assays adopting a singleplexing strategy often cumbersome, but they also typically required large sample volumes, especially when the targets to be analyzed are large in number.

A multiplex assay simultaneously measures multiple analytes (two or more) in a single assay. Multiplex assays are commonly used in high-throughput screening settings, where many specimens can be analyzed at once. It is the ability to assay many analytes simultaneously and many specimens in parallel that is the hallmark of multiplex assays and is the reason that such assays have become a powerful tool in fields ranging from drug discovery to functional genomics to clinical diagnostics. In contrast to singleplexing, by combining all targets in the same reaction vessel, the assay is much less cumbersome and much easier to perform, since only one reaction vessel is handled per sample. The required test samples can thus be dramatically reduced in volume, which is especially important when samples (e.g., tumor tissues, cerebral spinal fluid, or bone marrow) are difficult and/or invasive to retrieve in large quantities. Equally important is the fact that the reagent cost can be decreased and assay throughput increased drastically.

Many assays of complex macromolecule samples are composed of two steps. In the first step, agents capable of specifically capturing the target macromolecules are attached to a solid phase surface. These immobilized molecules may be used to capture the target macromolecules from a complex sample by various means, such as hybridization (e.g., in DNA, RNA based assays). In the second step, detection molecules are incubated with and bind to the complex of capture molecule and the target, emitting signals such as fluorescence or other electromagnetic signals. The amount of the target is then quantified by the intensity of those signals.

Multiplex assays may be carried out by utilizing multiple capture agents, each specific for a different target macromolecule. In chip-based array multiplex assays, each type of capture agent (e.g., a single-stranded oligonucleotide probe) is attached to a pre-defined position on the chip. The amount of multiplex targets in a complex sample is determined by measuring the signal of the detection molecule at each position corresponding to a type of capture agent. In suspension array multiplex assays, microparticles or microcarriers are suspended in the assay solution. These microparticles or microcarriers contain an identification element, which may be embedded, printed, or otherwise generated by one or more elements of the microparticle/microcarrier. Each type of capture agent is immobilized to particles with the same ID, and the signals emitted from the detection molecules on the surface of the particles with a particular ID reflect the amount of the corresponding target.

One application for which multiplex assays are particularly well-suited is detection of DNA and/or RNA mutations. In particular, detecting mutations associated with lung cancer can aid in early diagnosis and in identifying patients suitable for targeted therapies, depending on the genetic makeup of their cancers. However, existing diagnostic techniques are often expensive or time-consuming. Methods for detecting multiple gene mutations using serial, individual assays are time consuming and suffer from lack of uniformity if carried out using different assay types (see Schneider, M. et al. (2011) Cancers 3:91-105). Applying multiplex assay technologies such as analog-encoded microcarriers to this problem can provide cheaper, quicker assays with more accurate results while enabling multiplex screening for many mutations known to be correlated with tumorigenesis in a single assay.

Therefore, a need exists for applying a robust and sensitive multiplex assay system to the problem of screening for DNA and/or mutations. This provides a mechanism for multiplex detection of many DNA and/or RNA mutations in a single assay.

All publications, patents, and patent applications cited herein are hereby incorporated by reference in their entirety for all purposes.

### BRIEF SUMMARY

To meet this need, provided herein, inter alia, are methods and kits using microcarriers, encoded with a unique identifier, that include a probe for detecting a DNA or RNA mutation, e.g., a mutation associated with lung cancer. These microcarriers may be used in multiplexed assays in which each microcarrier includes a probe for detecting a particular mutation and an identifier for correlation of the microcarrier and its associated probe. The methods and kits disclosed herein may find use, e.g., in monitoring lung cancer, monitoring response to treatment of lung cancer, and/or early screening/detection of lung cancer.

Accordingly, in one aspect, provided herein is a method for detecting the presence of DNA mutations in the KRAS, NRAS, PIK3CA, BRAF, EGFR, AKT1, MEK1, and HER2 genes, the method comprising: (a) isolating DNA from a sample (e.g., obtained from a patient); (b) amplifying the isolated DNA by polymerase chain reaction (PCR) using primer pairs specific for the loci of one or more DNA mutations in each of the KRAS, NRAS, PIK3CA, BRAF, EGFR, AKT1, MEK1, and HER2 genes (e.g., in vitro); (c) hybridizing the amplified DNA with at least seven probes, said at least seven probes comprising one or more probes specific for a DNA mutation in each of the KRAS, NRAS, PIK3CA, BRAF, EGFR, AKT1, MEK1, and HER2 genes, wherein each of said at least seven probes is coupled to a microcarrier, and wherein each of the microcarriers comprises an identifier corresponding to the probe coupled thereto; (d) detecting presence or absence of hybridization of the amplified DNA with said at least seven probes, wherein hybridization between the amplified DNA and one of the probes indicates the presence of the DNA mutation corresponding to the probe; (e) detecting the identifiers of the microcarriers; and (f) correlating the detected identifiers of the microcarriers with the detected presence or absence of hybridization of the amplified DNA to the corresponding probes of the microcarriers. In some embodiments, the KRAS, NRAS, PIK3CA, BRAF, EGFR, AKT1, MEK1, and HER2 genes are human genes.

In some embodiments, step (b) comprises amplifying the isolated DNA by PCR in the presence of at least seven blocking nucleic acids, wherein each of said at least seven blocking nucleic acids hybridizes with a wild-type DNA locus corresponding with one of the DNA mutations in the KRAS, PIK3CA, BRAF, EGFR, AKT1, MEK1, or HER2 genes and prevents amplification of the wild-type DNA locus. In some embodiments, each of said at least seven blocking nucleic acids comprises: a single-stranded oligonucleotide that hybridizes with the corresponding wild-type DNA locus; and a 3′ terminal moiety that blocks extension from the single-stranded oligonucleotide. In some embodiments, the 3′ terminal moiety comprises one or more inverted deoxythymidines. In some embodiments, each of said at least seven blocking nucleic acids comprises one or more modified nucleotides selected from the group consisting of locked nucleic acids (LNAs), peptide nucleic acids (PNAs), hexose nucleic acids (HNAs), threose nucleic acids (TNAs), glycol nucleic acids (GNAs), and cyclohexenyl nucleic acids (CeNAs).

In some embodiments, the one or more DNA mutations in the KRAS gene comprise one or more DNA mutations encoding a G12D, G12V, or G12C mutated KRAS protein. In some embodiments, the one or more DNA mutations in the KRAS gene comprise DNA mutations encoding G12D, G12V, and G12C mutated KRAS proteins. In some embodiments, the probes specific for one or more DNA mutations in the KRAS gene comprise: (1) a first probe comprising a sequence selected from the group consisting of TAGTTGGAGCT (SEQ ID NO:38), TGTGGTAGTTG (SEQ ID NO:40), TGATGGCGTAG (SEQ ID NO:42), TGGAGCTGATGGC (SEQ ID NO:44), and GCGTAGGCAAG (SEQ ID NO:46); (2) a second probe comprising a sequence selected from the group consisting of CTGTTGGCGTAGG (SEQ ID NO:48), GTAGTTGGAGCTG (SEQ ID NO:50), TGGAGCTGTTGGC (SEQ ID NO:52), TTGTGGTAGTTGG (SEQ ID NO:54), and GGCGTAGGCAAGA (SEQ ID NO:56); and (3) a third probe comprising a sequence selected from the group consisting of TAGTTGGAGCTT (SEQ ID NO:58), GCGTAGGCAAGA (SEQ ID NO:60), GGAGCTTGTGGC (SEQ ID NO:62), TTGTGGCGTAGG (SEQ ID NO:64), and TGTGGTAGTTGG (SEQ ID NO:66); wherein each of the three probes is coupled to a microcarrier with a different identifier. In some embodiments, each of the three probes further comprises eight nucleotides at the 5′ end, and wherein the eight nucleotides at the 5′ end are adenine or thymine nucleotides. In some embodiments, the probes specific for one or more DNA mutations in the KRAS gene comprise: (1) a first probe comprising a sequence selected from the group consisting of TTTTTTTTTTTTAATAGTTGGAGCT (SEQ ID NO:39), TTTTTTTTTTTTAATGTGGTAGTTG (SEQ ID NO:41), TTTTTTTTTTTTAATGATGGCGTAG (SEQ ID NO: 43), TTTTTTTTTTTATGGAGCTGATGGC (SEQ ID NO:45), and TTTTTTTTTTTTAAGCGTAGGCAAG (SEQ ID NO:47); (2) a second probe comprising a sequence selected from the group consisting of TTTTTTTTTTTACTGTTGGCGTAGG (SEQ ID NO:49), TTTTTTTTTTTAGTAGTTGGAGCTG (SEQ ID NO:51), TTTTTTTTTTTATGGAGCTGTTGGC (SEQ ID NO:53), TTTTTTTTTTTATTGTGGTAGTTGG (SEQ ID NO:55), and TITTTTTTTTTAGGCGTAGGCAAGA (SEQ ID NO:57); and (3) a third probe comprising a sequence selected from the group consisting of TTTTTTTTTTTAATAGTTGGAGCTT (SEQ ID NO:59), TTTTTTTTTTTAAGCGTAGGCAAGA (SEQ ID NO:61), TTTTTTTTTTTAAGGAGCTTGTGGC (SEQ ID NO: 63), TTTTTTTTTTTAATTGTGGCGTAGG (SEQ ID NO: 65), and TTTTTTTTTTTAATGTGGTAGTTGG (SEQ ID NO:67); wherein each of the three probes is coupled to a microcarrier with a different identifier. In some embodiments, step (b) comprises amplifying the isolated DNA by PCR using a primer pair comprising the sequences GTACTGGTGGAGTATTTGATAGTG (SEQ ID NO:1) and CGTCAAGGCACTCTTGCCTAC (SEQ ID NO:2). In some embodiments, step (b) comprises amplifying the isolated DNA by PCR in the presence of a blocking nucleic acid that hybridizes with a wild-type KRAS DNA locus corresponding with one of the KRAS DNA mutations and prevents amplification of the wild-type KRAS DNA locus, and wherein the blocking nucleic acid comprises the sequence TACGCCACCAGCT(invdT)ₙ, wherein n is 1, 2, or 3 (SEQ ID NO:281); TTGGAGCTGGTGGCGTA(invdT)ₙ, wherein n is 1, 2, or 3 (SEQ ID NO:282); GCTGGTGGCGTAGGCA(invdT)ₙ, wherein n is 1, 2, or 3 (SEQ ID NO:283); GCTGGTGGCGTAGGC(invdT)ₙ, wherein n is 1, 2, or 3 (SEQ ID NO:284); or TTGGAGCTGGTGGCGT(invdT)ₙ, wherein n is 1, 2, or 3 (SEQ ID NO:285); with italicized nucleic acids representing locked nucleic acids.

In some embodiments, the one or more DNA mutations in the PIK3CA gene comprise one or more DNA mutations encoding an E542K or E545K mutated PIK3CA protein. In some embodiments, the one or more DNA mutations in the PIK3CA gene comprise DNA mutations encoding E542K and E545K mutated PIK3CA proteins. In some embodiments, the probes specific for one or more DNA mutations in the PIK3CA gene comprise: (1) a first probe comprising a sequence selected from the group consisting of GCTCAGTGATTTTAG (SEQ ID NO:87), TGCTCAGTGATTTT (SEQ ID NO:89), GCTCAGTGATTTTAG (SEQ ID NO: 91), CCTGCTCAGTGATTTTA (SEQ ID NO:93), and CTCAGTGATTTTAGA (SEQ ID NO:95); and (2) a second probe comprising a sequence selected from the group consisting of TTCTCCTGCTTA (SEQ ID NO:97), CTCCTGCTTAGT (SEQ ID NO:99), TCTCCTGCTTAG (SEQ ID NO:101), TCCTGCTTAGTG (SEQ ID NO:103), and CTCCTGCTTAGTGA (SEQ ID NO:105); wherein each of the two probes is coupled to a microcarrier with a different identifier. In some embodiments, each of the two probes further comprises eight nucleotides at the 5′ end, and wherein the eight nucleotides at the 5′ end are adenine or thymine nucleotides. the probes specific for one or more DNA mutations in the PIK3CA gene comprise: (1) a first probe comprising a sequence selected from the group consisting of TTTTTTTTTAGCTCAGTGATTTTAG (SEQ ID NO:88), TTTTTTTTTTGCTCAGTGATTTT (SEQ ID NO:90), TTTTTTTTTAGCTCAGTGATTTTAG (SEQ ID NO:92), TTTTTTTCCTGCTCAGTGATTTTA (SEQ ID NO:94), and TTTTTTTTTTTCTCAGTGATTTTAGA (SEQ ID NO:96); and (2) a second probe comprising a sequence selected from the group consisting of TTTTTTTTTTTTTCTCCTGCTTA (SEQ ID NO:98), TTTTTTTTTTTTTCTCCTGCTTAGT (SEQ ID NO: 100), TTTTTTTTTTTATCTCCTGCTTAG (SEQ ID NO:102), TTTTTTTTTTTTTTTCCTGCTTAGTG (SEQ ID NO:104), and TTTTTTTTTTTTTCTCCTGCTTAGTGA (SEQ ID NO:106); wherein each of the three probes is coupled to a microcarrier with a different identifier. In some embodiments, step (b) comprises amplifying the isolated DNA by PCR using a primer pair comprising the sequences CAATTTCTACAAGAGATCCTCTCTCT (SEQ ID NO:5) and CTCCATTTTAGCACTTACCTGTGAC (SEQ ID NO:6). In some embodiments, step (b) comprises amplifying the isolated DNA by PCR in the presence of a blocking nucleic acid that hybridizes with a wild-type PIK3CA DNA locus corresponding with one of the PIK3CA DNA mutations and prevents amplification of the wild-type PIK3CA DNA locus, and wherein at least one of the at least seven blocking nucleic acids comprises the sequence CTGAAATCACTGAGCAGG(invdT)ₙ, wherein n is 1, 2, or 3 (SEQ ID NO:291); TCTCTGAAATCACTGAGCAGG(invdT)ₙ, wherein n is 1, 2, or 3 (SEQ ID NO:292); TCTCTGAAATCACTGAGCAGG(invdT)ₙ, wherein n is 1, 2, or 3 (SEQ ID NO:293); TCTCTGAAATCACTGAGCAGG(invdT)ₙ, wherein n is 1, 2, or 3 (SEQ ID NO:294); or TCTCTGAATTCACTGAGCAGG(invdT)ₙ, wherein n is 1, 2, or 3 (SEQ ID NO:295); with italicized nucleic acids representing locked nucleic acids. In some embodiments, the one or more DNA mutations in the PIK3CA gene comprise a DNA mutation encoding an H1047R mutated PIK3CA protein. In some embodiments, the probes specific for one or more DNA mutations in the PIK3CA gene comprise: (1) a first probe comprising a sequence selected from the group consisting of GATGCACGTCATG (SEQ ID NO:107), TGAATGATGCACG (SEQ ID NO:109), TGATGCACGTC (SEQ ID NO:111), AATGATGCACGTCA (SEQ ID NO:113), and AATGATGCACGTC (SEQ ID NO:115); wherein the first probe is coupled to a microcarrier with an identifier. In some embodiments, the first probe further comprises eight nucleotides at the 5′ end, and wherein the eight nucleotides at the 5′ end are adenine or thymine nucleotides. In some embodiments, the probes specific for one or more DNA mutations in the PIK3CA gene comprise: (1) a first probe comprising a sequence selected from the group consisting of TTTTTTTTTTTTTGATGCACGTCATG (SEQ ID NO:108), TTTTTTTTTTTGAATGATGCACG (SEQ ID NO:110), TTTTTTTTTTTTTGATGCACGTC (SEQ ID NO:112), TTTTTTTTTTTTAATGATGCACGTCA (SEQ ID NO:114), and TTTTTTTTTTTTAATGATGCACGTC (SEQ ID NO:116); wherein the first probe is coupled to a microcarrier with an identifier. In some embodiments, step (b) comprises amplifying the isolated DNA by PCR using a primer pair comprising the sequences ACCCTAGCCTTAGATAAAACTGAGC (SEQ ID NO:7) and TTTGTTGTCCAGCCACCATGA (SEQ ID NO:8). In some embodiments, step (b) comprises amplifying the isolated DNA by PCR in the presence a blocking nucleic acid that hybridizes with a wild-type PIK3CA DNA locus corresponding with one of the PIK3CA DNA mutations and prevents amplification of the wild-type PIK3CA DNA locus, and wherein at least one of the at least seven blocking nucleic acids comprises the sequence CACCATGATGTGCAT(invdT)ₙ, wherein n is 1, 2, or 3 (SEQ ID NO:296); CCACCATGATGTGCAT(invdT)ₙ, wherein n is 1, 2, or 3 (SEQ ID NO:297); CACCATGATGTGCAT(invdT)ₙ, wherein n is 1, 2, or 3 (SEQ ID NO:298); CCACCATGATGTGCATCA(invdT)ₙ, wherein n is 1, 2, or 3 (SEQ ID NO:299); or CATGATGTGCA(invdT)ₙ, wherein n is 1, 2, or 3 (SEQ ID NO:300); with italicized nucleic acids representing locked nucleic acids.

In some embodiments, the one or more DNA mutations in the BRAF gene comprise one or more DNA mutations encoding a V600E mutated BRAF protein. In some embodiments, the probe specific for one or more DNA mutations in the BRAF gene comprises a sequence selected from the group consisting of TTTGGTCTAGCTACAGA (SEQ ID NO:79), CTACAGAGAAATCTCGA (SEQ ID NO:81), GTGATTTTGGTCTAGCT (SEQ ID NO:83), and TCTAGCTACAGAGAAAT (SEQ ID NO:85). In some embodiments, the probe specific for one or more DNA mutations in the BRAF gene further comprises eight nucleotides at the 5′ end, and wherein the eight nucleotides at the 5′ end are adenine or thymine nucleotides. In some embodiments, the probe specific for one or more DNA mutations in the BRAF gene comprises a sequence selected from the group consisting of TTTTTTAATTGAGAAATCTCGATGGAG (SEQ ID NO:78), TTTTTTAATTTTTGGTCTAGCTACAGA (SEQ ID NO:80), TTTTTTAATTCTACAGAGAAATCTCGA (SEQ ID NO:82), TTTTTTAATTGTGATTTTGGTCTAGCT (SEQ ID NO:84), and TTTTTTAATTTCTAGCTACAGAGAAAT (SEQ ID NO:86). In some embodiments, step (b) comprises amplifying the isolated DNA by PCR using a primer pair comprising the sequences ATAGCCTCAATTCTTACCATCCACAAAATG (SEQ ID NO:9) and CAGATATATTTCTTCATGAAGACCTCACAGTAA (SEQ ID NO:10). In some embodiments, step (b) comprises amplifying the isolated DNA by PCR in the presence of a blocking nucleic acid that hybridizes with a wild-type BRAF DNA locus corresponding with the BRAF DNA mutation and prevents amplification of the wild-type BRAF DNA locus, and wherein at least one of the at least seven blocking nucleic acids comprises the sequence GAGATTTCACTGTAGC(invdT)ₙ, wherein n is 1, 2, or 3 (SEQ ID NO:301); GAGATTTCACTGTAGC(invdT)ₙ, wherein n is 1, 2, or 3 (SEQ ID NO:302); GAGAITTCACTGTAGC(invdT)ₙ, wherein n is 1, 2, or 3 (SEQ ID NO:303); GAGATTTCACTGTAGC(invdT)ₙ, wherein n is 1, 2, or 3 (SEQ ID NO:304); or GAGATTTCACTGTAGC(invdT)ₙ, wherein n is 1, 2, or 3 (SEQ ID NO:305); with italicized nucleic acids representing locked nucleic acids.

In some embodiments, the one or more DNA mutations in the EGFR gene comprise one or more DNA mutations encoding a G719A mutated EGFR protein. In some embodiments, the probe specific for one or more DNA mutations in the EGFR gene comprises a sequence selected from the group consisting of TCAAAGTGCTGGCCTC (SEQ ID NO:117), AGATCAAAGTGCTGGCCTCCG (SEQ ID NO:119), AAAGTGCTGGCCT (SEQ ID NO:121), AGTGCTGGCCT (SEQ ID NO:123), and AAGTGCTGGCCTC (SEQ ID NO:125). In some embodiments, the probe specific for one or more DNA mutations in the EGFR gene further comprises eight nucleotides at the 5′ end, and wherein the eight nucleotides at the 5′ end are adenine or thymine nucleotides. In some embodiments, the probe specific for one or more DNA mutations in the EGFR gene comprises a sequence selected from the group consisting of TTTTTTTTTTCAAAGTGCTGGCCTC (SEQ ID NO:118), TTTTTTAGATCAAAGTGCTGGCCTCCG (SEQ ID NO:120), TTTTTTTTTTTTAAAGTGCTGGCCT (SEQ ID NO:122), TTTTTTTTTTTTTAGTGCTGGCCT (SEQ ID NO:124), and TTTTTTTTTTTTAAGTGCTGGCCTC (SEQ ID NO:126). In some embodiments, step (b) comprises amplifying the isolated DNA by PCR using a primer pair comprising the sequences CTTGTGGAGCCTCTTACACCC (SEQ ID NO:11) and TGCCGAACGCACCGGA (SEQ ID NO:12). In some embodiments, step (b) comprises amplifying the isolated DNA by PCR in the presence of a blocking nucleic acid that hybridizes with a wild-type EGFR DNA locus corresponding with the EGFR DNA mutation and prevents amplification of the wild-type EGFR DNA locus, and wherein at least one of the at least seven blocking nucleic acids comprises the sequenceCGGAGCCCAGCACTTTGA (invdT)ₙ, wherein n is 1, 2, or 3 (SEQ ID NO:306); CGCACCGGAGCCCAGCACT (invdT)ₙ, wherein n is 1, 2, or 3 (SEQ ID NO:307); GAGCCCAGCAC (invdT)ₙ, wherein n is 1, 2, or 3 (SEQ ID NO:308); CGCACCGGAGCCCAGCAC (invdT)ₙ, wherein n is 1, 2, or 3 (SEQ ID NO:309); or CGCACCGGAGCCCAGCACTTA (invdT)ₙ, wherein n is 1, 2, or 3 (SEQ ID NO:310); with italicized nucleic acids representing locked nucleic acids. In some embodiments, the one or more DNA mutations in the EGFR gene comprise one or more DNA mutations encoding an E746_A750del mutated EGFR protein. In some embodiments, the probes specific for one or more DNA mutations in the EGFR gene comprise: (1) a first probe comprising a sequence selected from the group consisting of AATCAAAACATCTCCGAAAG (SEQ ID NO:128), CAAAACATCTCCG (SEQ ID NO:130), AACATCTCCG (SEQ ID NO:132), and AAACATCTCCGAAAGCC (SEQ ID NO:134); and (2) a second probe comprising a sequence selected from the group consisting of AATCAAGACATCTCCGA (SEQ ID NO:136), GCAATCAAGACATCTCCGA (SEQ ID NO:138), AATCAAGACATCTC (SEQ ID NO:140), AATCAAGACATCTCCGAAAGC (SEQ ID NO:142), and CAAGACATCTCCGA (SEQ ID NO:144); wherein each of the two probes is coupled to a microcarrier with a different identifier. In some embodiments, each of the two probes further comprises eight nucleotides at the 5′ end, and wherein the eight nucleotides at the 5′ end are adenine or thymine nucleotides. In some embodiments, the probes specific for one or more DNA mutations in the EGFR gene comprise: (1) a first probe comprising a sequence selected from the group consisting of TTTTTTTTTAATCAAAACATCTCCG (SEQ ID NO:127), TTTTTTTTTAATCAAAACATCTCCGAAAG (SEQ ID NO:129), TTTTTTTTTTTACAAAACATCTCCG (SEQ ID NO:131), TTTTTTTTTTTTTTTAACATCTCCG (SEQ ID NO:133), and TTTTTTTTTTTTTTAAACATCTCCGAAAGCC (SEQ ID NO:135); and (2) a second probe comprising a sequence selected from the group consisting of TTTTTTTTAATCAAGACATCTCCGA (SEQ ID NO:137), TTTTTTGCAATCAAGACATCTCCGA (SEQ ID NO:139), TTTTTTTTAATCAAGACATCTC (SEQ ID NO:141), TTTTTTTTAATCAAGACATCTCCGAAAGC (SEQ ID NO:143), and TTTTTTTTTTTCAAGACATCTCCGA (SEQ ID NO: 145); wherein each of the two probes is coupled to a microcarrier with a different identifier. In some embodiments, step (b) comprises amplifying the isolated DNA by PCR using a primer pair comprising the sequences GCCAGTTAACGTCTTCCTTCTC (SEQ ID NO:13) and ATCGAGGATTTCCTTGTTGGCTT (SEQ ID NO: 14). In some embodiments, step (b) comprises amplifying the isolated DNA by PCR in the presence of a blocking nucleic acid that hybridizes with a wild-type EGFR DNA locus corresponding with the EGFR DNA mutation and prevents amplification of the wild-type EGFR DNA locus, and wherein at least one of the at least seven blocking nucleic acids comprises the sequence CGGAGATGTTGCTTCTCTTAATTCC(invdT)ₙ, wherein n is 1, 2, or 3 (SEQ ID NO:311); CGGAGATGTTGCTTCTCT(invdT)ₙ, wherein n is 1, 2, or 3 (SEQ ID NO: 312); GTTGCTTCTCTTAATTCC(invdT)ₙ, wherein n is 1, 2, or 3 (SEQ ID NO: 313); ATGTTGCTTCTCT(invdT)ₙ, wherein n is 1, 2, or 3 (SEQ ID NO:314); or TTGCTTCTCTTA(invdT)ₙ, wherein n is 1, 2, or 3 (SEQ ID NO:315); with italicized nucleic acids representing locked nucleic acids. In some embodiments, the one or more DNA mutations in the EGFR gene comprise one or more DNA mutations encoding a T790M, C797S, S768I, V769_D770insASV, H773_V774insH, D770_N771insG, or D770_N771insSVD mutated EGFR protein. In some embodiments, the one or more DNA mutations in the EGFR gene comprise DNA mutations encoding T790M, C797S, S768I, V769_D770insASV, H773_V774insH, D770_N771insG, and D770_N771insSVD mutated EGFR proteins. In some embodiments, the probes specific for one or more DNA mutations in the EGFR gene comprise: (1) a first probe comprising a sequence selected from the group consisting of GAGATGCATGATGA (SEQ ID NO:146), TGAGATGCATGATGAG (SEQ ID NO:147), ATGAGATGCATGATGAG (SEQ ID NO:148), TGAGCTGCATGATGA (SEQ ID NO:149), and CATGAGATGCATGATGA (SEQ ID NO:150); (2) a second probe comprising a sequence selected from the group consisting of CCAGGAGGCTGCCG (SEQ ID NO:461), CAGGAGGCTGCCGA (SEQ ID NO:463), TCCAGGAGGCTGCC (SEQ ID NO:465), CCAGGAGGCTGCC (SEQ ID NO:467), and CAGGAGGCTGCC (SEQ ID NO:469); (3) a third probe comprising a sequence selected from the group consisting of CCAGGAGGGAGCC (SEQ ID NO:471), CCAGGAGGGAGCCG (SEQ ID NO:473), TCCAGGAGGGAGCC (SEQ ID NO:475), CAGGAGGGAGCCG (SEQ ID NO:477), and CAGGAGGGAGCCGA (SEQ ID NO:479); (4) a fourth probe comprising a sequence selected from the group consisting of ATGGCCATCTTGG (SEQ ID NO:421), GGCCATCTTGGA (SEQ ID NO:423), GATGGCCATCTTG (SEQ ID NO:425), TGATGGCCATCTTG (SEQ ID NO:427), and TGGCCATCTTGG (SEQ ID NO:429); (5) a fifth probe comprising a sequence selected from the group consisting of GTGATGGCCGG (SEQ ID NO:431), TGATGGCCGGCG (SEQ ID NO:433), GTGATGGCCGGCGT (SEQ ID NO:435), GATGGCCGGCGT (SEQ ID NO:437), and GATGGCCCGCGTG (SEQ ID NO:439); (6) a sixth probe comprising a sequence selected from the group consisting of AACCCCCATCACGT (SEQ ID NO:441), GACAACCCCCATCACG (SEQ ID NO:443), CGTGGACAACCCCCATCA (SEQ ID NO:445), CCCATCACGTGT (SEQ ID NO:447), and TGGACAACCCCCATCAC (SEQ ID NO:449); and (7) a seventh probe comprising a sequence selected from the group consisting of GCCAGCGTGGACGG (SEQ ID NO:451), CGTGGACGGTAACC (SEQ ID NO:453), GACGGTAACCCCC (SEQ ID NO:455), CCAGCGTGGACGGT (SEQ ID NO:457), and GCCAGCGTGGACGGTA (SEQ ID NO:459); wherein each of the seven probes is coupled to a microcarrier with a different identifier. In some embodiments, each of the seven probes specific for one or more DNA mutations in the EGFR gene further comprises eight nucleotides at the 5′ end, and wherein the eight nucleotides at the 5′ end are adenine or thymine nucleotides. In some embodiments, the probes specific for one or more DNA mutations in the EGFR gene comprise: (1) a first probe comprising a sequence selected from the group consisting of TTTTTTTTTTTGAGATGCATGATGA (SEQ ID NO:352), TTTTTTTTTTGAGATGCATGATGAG (SEQ ID NO:353), TTTTTTTTATGAGATGCATGATGAG (SEQ ID NO:354), TTTTTTTTTTTGAGCTGCATGATGA (SEQ ID NO:355), and TTTTTTTTCATGAGATGCATGATGA (SEQ ID NO:356); (2) a second probe comprising a sequence selected from the group consisting of TTTTTTTTTTTACCAGGAGGCTGCCG (SEQ ID NO:462), TTTTTTTTTTTACAGGAGGCTGCCGA (SEQ ID NO:464), TTTTTTTTTTTATCCAGGAGGCTGCC (SEQ ID NO:466), TTTTTTTTTTTACCAGGAGGCTGCC (SEQ ID NO:468), and TTTTTTTTTTTACAGGAGGCTGCC (SEQ ID NO:470); (3) a third probe comprising a sequence selected from the group consisting of TTTTTTTTTTTACCAGGAGGGAGCC (SEQ ID NO:472), TTTTTTTTTTTACCAGGAGGGAGCCG (SEQ ID NO:474), TTTTTTTTTTTATCCAGGAGGGAGCC (SEQ ID NO:476), TTTTTTTTTTTACAGGAGGGAGCCG (SEQ ID NO:478), and TTTTTTTTTTACAGGAGGGAGCCGA (SEQ ID NO:480); (4) a fourth probe comprising a sequence selected from the group consisting of TTTTTTTTTATGGCCATCTTGG (SEQ ID NO:422), TTTTTTTTTTAGGCCATCTTGGA (SEQ ID NO:424), TTTTTTTAGATGGCCATCTTG (SEQ ID NO:426), TTTTTTTTGATGGCCATCTTG (SEQ ID NO:428), and TTTTTTTTTTTGGCCATCTTGG (SEQ ID NO:430), (5) a fifth probe comprising a sequence selected from the group consisting of TTTTTTTTTTTGTGATGGCCGG (SEQ ID NO:432), TTTTTTTTTTTTTGATGGCCGGCG (SEQ ID NO:434), TTTTTTTTTTTGTGATGGCCGGCGT (SEQ ID NO:436), TTTTTTTTTTTTTGATGGCCGGCGT (SEQ ID NO:438), and TTTTTTTTTTTTTGATGGCCCGCGTG (SEQ ID NO:440); (6) a sixth probe comprising a sequence selected from the group consisting of TTTTTTTTTTTAACCCCCATCACGT (SEQ ID NO:442), TTTTTTTTGACAACCCCCATCACG (SEQ ID NO:444), TTTTCGTGGACAACCCCCATCA (SEQ ID NO:446), TTTTTTTTTTTTCCCATCACGTGT (SEQ ID NO:448), and TTTTTTTTGGACAACCCCCATCAC (SEQ ID NO:450); and (7) a seventh probe comprising a sequence selected from the group consisting of TTTTTTTTTTTGCCAGCGTGGACGG (SEQ ID NO:452), TTTTTTTTTTTCGTGGACGGTAACC (SEQ ID NO:454), TTTTTTTTTTTGACGGTAACCCCC (SEQ ID NO:456), TTTTTTTTTTCCAGCGTGGACGGT (SEQ ID NO:458), and TTTTTTTGCCAGCGTGGACGGTA (SEQ ID NO:460); wherein each of the seven probes is coupled to a microcarrier with a different identifier. In some embodiments, step (b) comprises amplifying the isolated DNA by PCR using a primer pair comprising the sequences CCTCCACCGTGCAGATCATC (SEQ ID NO:15) and TTCCCTGATTACCTTTGCGAT (SEQ ID NO:16); a primer pair comprising the sequences CCACACTGACGTGCCTCT (SEQ ID NO:511) and GCACACGTAGGGGTTGTCCAAGA (SEQ ID NO:512); a primer pair comprising the sequences CCACACTGACGTGCCTCT (SEQ ID NO:513) and GTACACGCTGGCCACGCCG (SEQ ID NO:514); a primer pair comprising the sequences CCACACTGACGTGCCTCT (SEQ ID NO:515) and CAGGCGGCACACGTGAT (SEQ ID NO:516); and/or a primer pair comprising the sequences CCACACTGACGTGCCTCT (SEQ ID NO:517) and AGGCGGCACACGTGCGGGTTAC (SEQ ID NO:518). In some embodiments, step (b) comprises amplifying the isolated DNA by PCR in the presence of a blocking nucleic acid that hybridizes with a wild-type EGFR DNA locus corresponding with the EGFR DNA mutation and prevents amplification of the wild-type EGFR DNA locus, and wherein at least one of the at least seven blocking nucleic acids comprises the sequence CATCACGCAGCTCATG(invdT)ₙ, wherein n is 1, 2, or 3 (SEQ ID NO:316); TGCAGCTCATCACGCAGC(invdT)ₙ, wherein n is 1, 2, or 3 (SEQ ID NO:317); TCATCACGCAGCTCAT(invdT)ₙ, wherein n is 1, 2, or 3 (SEQ ID NO:318); TCATCACGCAGC(invdT)ₙ, wherein n is 1, 2, or 3 (SEQ ID NO: 319); or CTCATCACGCAGC(invdT)ₙ, wherein n is 1, 2, or 3 (SEQ ID NO:320); with italicized nucleic acids representing locked nucleic acids. In some embodiments, the one or more DNA mutations in the EGFR gene comprise one or more DNA mutations encoding an L858R mutated EGFR protein. In some embodiments, the probes specific for one or more DNA mutations in the EGFR gene comprise: a first probe comprising a sequence selected from the group consisting of ATTTTGGGCGGGCC (SEQ ID NO:151), TTGGGCGGGCCAAA (SEQ ID NO: 153), GCGGGCCAAACT (SEQ ID NO: 155), GGGCGGGCCAAACT (SEQ ID NO:157), and TGGGCGGGCCA (SEQ ID NO:159); wherein the first probe is coupled to a microcarrier with an identifier. In some embodiments, the first probe further comprises eight nucleotides at the 5′ end, and wherein the eight nucleotides at the 5′ end are adenine or thymine nucleotides. In some embodiments, the probes specific for one or more DNA mutations in the EGFR gene comprise: a first probe comprising a sequence selected from the group consisting of TTTTTTTATTTTGGGCGGGCC (SEQ ID NO:152), TTTTTTTTAATTGGGCGGGCCAAA (SEQ ID NO:154), TTTTTTTAAAAAAGCGGGCCAAACT (SEQ ID NO: 156), TTTTTTTTAAAAGGGCGGGCCAAACT (SEQ ID NO:158), and TTTTTTTTAAATGGGCGGGCCA (SEQ ID NO:160); wherein the first probe is coupled to a microcarrier with an identifier. In some embodiments, step (b) comprises amplifying the isolated DNA by PCR using a primer pair comprising the sequences GGAGGACCGTCGCTTGG (SEQ ID NO:17) and TCTTTCTCTTCCGCACCCAG (SEQ ID NO:18). In some embodiments, step (b) comprises amplifying the isolated DNA by PCR in the presence of a blocking nucleic acid that hybridizes with a wild-type EGFR DNA locus corresponding with the EGFR DNA mutation and prevents amplification of the wild-type EGFR DNA locus, and wherein at least one of the at least seven blocking nucleic acids comprises the sequence CCAGCAGTTTGGCCAGCCCT(invdT)ₙ, wherein n is 1, 2, or 3 (SEQ ID NO:321); CCAGCAGTTTGGCCAGCCCT(invdT)ₙ, wherein n is 1, 2, or 3 (SEQ ID NO:322); CCAGCAGTTTGGCCAGCCCT(invdT)ₙ, wherein n is 1, 2, or 3 (SEQ ID NO:323); AGCAGTTTGGCCAGCC(invdT)ₙ, wherein n is 1, 2, or 3 (SEQ ID NO:324); or CCAGCAGTTTGGCCAGCCCT(invdT)ₙ, wherein n is 1, 2, or 3 (SEQ ID NO:325); with italicized nucleic acids representing locked nucleic acids.

In some embodiments, the one or more DNA mutations in the AKT1 gene comprise one or more DNA mutations encoding an E17K mutated AKT1 protein. In some embodiments, the probe specific for one or more DNA mutations in the AKT1 gene comprises a sequence selected from the group consisting of TGTAGGGAAGTACA (SEQ ID NO:370), TCTGTAGGGAAGTAC (SEQ ID NO:372), GTCTGTAGGGAAGTACAT (SEQ ID NO:374), CCGCACGTCTGTAGGGA (SEQ ID NO:376), and ACGTCTGTAGGGAAGTA (SEQ ID NO:378). In some embodiments, the probe specific for one or more DNA mutations in the AKT1 gene further comprises seven nucleotides at the 5′ end, and wherein the seven nucleotides at the 5′ end are adenine or thymine nucleotides. In some embodiments, the probe specific for one or more DNA mutations in the AKT1 gene comprises a sequence selected from the group consisting of TTTTTTTTTTTTTTGTAGGGAAGTACA (SEQ ID NO:371), TTTTTTTTTTTTCTGTAGGGAAGTAC (SEQ ID NO:373), TTTTTTTGTCTGTAGGGAAGTACAT (SEQ ID NO:375), TTTTTTTCCGCACGTCTGTAGGGA (SEQ ID NO:377), and TTTTTTTTACGTCTGTAGGGAAGTA (SEQ ID NO:379). In some embodiments, step (b) comprises amplifying the isolated DNA by PCR using a primer pair comprising the sequences GAGGGTCTGACGGGTAGAGTG (SEQ ID NO:380) and TGGCCGCCAGGTCTTGATGTA (SEQ ID NO:381). In some embodiments, step (b) comprises amplifying the isolated DNA by PCR in the presence of a blocking nucleic acid that hybridizes with a wild-type AKT1 DNA locus corresponding with the AKT1 DNA mutation and prevents amplification of the wild-type AKT1 DNA locus, and wherein at least one of the at least seven blocking nucleic acids comprises the sequence TGTACTCCCCTACA (invdT)ₙ, wherein n is 1, 2, or 3 (SEQ ID NO:382); GATGTACTCCCCT (invdT)ₙ, wherein n is 1, 2, or 3 (SEQ ID NO:383); ATGTACTCCCCTAC (invdT)ₙ, wherein n is 1, 2, or 3 (SEQ ID NO:384); GTACTCCCCTACA (invdT)ₙ, wherein n is 1, 2, or 3 (SEQ ID NO:385); or GATGTACTCCCCTACA (invdT)ₙ, wherein n is 1, 2, or 3 (SEQ ID NO:386); with italicized nucleic acids representing locked nucleic acids.

In some embodiments, the one or more DNA mutations in the MEK1 gene comprise one or more DNA mutations encoding a K57N mutated MEK1 protein. In some embodiments, the probe specific for one or more DNA mutations in the MEK1 gene comprises a sequence selected from the group consisting of TTACCCAGAATCAGAA (SEQ ID NO:387), CCAGAATCAGAAGGTG (SEQ ID NO:389), TTCTTACCCAGAATCA (SEQ ID NO:391), CCTTTCTTACCCAGAATC (SEQ ID NO:393), and CAGAATCAGAAGGTGG (SEQ ID NO:395). In some embodiments, the probe specific for one or more DNA mutations in the MEK1 gene further comprises eight nucleotides at the 5′ end, and wherein the eight nucleotides at the 5′ end are adenine or thymine nucleotides. In some embodiments, the probe specific for one or more DNA mutations in the MEK1 gene comprises a sequence selected from the group consisting of TTTTTAAATTTACCCAGAATCAGAA (SEQ ID NO:388), TTTTTAAATCCAGAATCAGAAGGTG (SEQ ID NO:390), TTTTTAAATTTCTTACCCAGAATCA (SEQ ID NO:392), TTTTTAAATCCTTTCTTACCCAGAATC (SEQ ID NO:394), and TTTTTAAATCAGAATCAGAAGGTGG (SEQ ID NO:396). In some embodiments, step (b) comprises amplifying the isolated DNA by PCR using a primer pair comprising the sequences CTTGATGAGCAGCAGCGAAA (SEQ ID NO:397) and CCTTCAGTTCTCCCACCTTCTG (SEQ ID NO:398). In some embodiments, step (b) comprises amplifying the isolated DNA by PCR in the presence of a blocking nucleic acid that hybridizes with a wild-type MEK1 DNA locus corresponding with the MEK1 DNA mutation and prevents amplification of the wild-type MEK1 DNA locus, and wherein at least one of the at least seven blocking nucleic acids comprises the sequence TCTGCTTCTGGGTAAG (invdT)ₙ, wherein n is 1, 2, or 3 (SEQ ID NO:399); TTCTGCTTCTGGGTAAGA (invdT)ₙ, wherein n is 1, 2, or 3 (SEQ ID NO:400); CACCTTCTGCTTCTGGG (invdT)ₙ, wherein n is 1, 2, or 3 (SEQ ID NO:401); TCTGCTTCTGGGTA (invdT)ₙ, wherein n is 1, 2, or 3 (SEQ ID NO:402); or CACCTTCTGCTTCTGGGTAAGA (invdT)ₙ, wherein n is 1, 2, or 3 (SEQ ID NO:403); with italicized nucleic acids representing locked nucleic acids.

In some embodiments, the one or more DNA mutations in the HER2 gene comprise one or more DNA mutations encoding an A775_G776insYVMA mutated HER2 protein. In some embodiments, the probe specific for one or more DNA mutations in the HER2 gene comprises a sequence selected from the group consisting of ATACGTGATGTCTTAC (SEQ ID NO:404), ACGTGATGGCTTACGT (SEQ ID NO:406), AAGCATACGTGATGGCT (SEQ ID NO:408), GCATACGTGATGGCTT (SEQ ID NO:410), and GCATACGTGATGGCTTA (SEQ ID NO:412). In some embodiments, the probe specific for one or more DNA mutations in the HER2 gene further comprises five nucleotides at the 5′ end, and wherein the five nucleotides at the 5′ end are adenine or thymine nucleotides. In some embodiments, the probe specific for one or more DNA mutations in the HER2 gene comprises a sequence selected from the group consisting of TTTTTTTTTATACGTGATGTCTTAC (SEQ ID NO:405), TTTTTTTTTTTACGTGATGGCTTACGT (SEQ ID NO:407), TTTTTAAGCATACGTGATGGCT (SEQ ID NO:409), TTTTTTTGCATACGTGATGGCTT (SEQ ID NO:411), and TTTTTTTGCATACGTGATGGCTTA (SEQ ID NO:413). In some embodiments, step (b) comprises amplifying the isolated DNA by PCR using a primer pair comprising the sequences ATGGCTGTGGTTTGTGATGGT (SEQ ID NO:414) and ACACCAGCCATCACGTAAGACA (SEQ ID NO:415).

In some embodiments according to any of the embodiments described herein, the sample is a blood, serum, or plasma sample. In some embodiments, (a) comprises isolating circulating free DNA (cfDNA) from the sample, and wherein the isolated cfDNA is amplified by PCR in (b). In some embodiments, the methods further comprise amplifying a positive control DNA sequence using a primer pair specific for the positive control DNA sequence in (b); hybridizing the amplified positive control gene sequence with a probe specific for the positive control gene sequence in (c), wherein the probe specific for the positive control gene sequence is coupled to a microcarrier with an identifier corresponding to a positive control; detecting presence or absence of hybridization of the amplified positive control DNA sequence with the probe specific for the positive control gene sequence in (d); and detecting the identifier corresponding to the positive control in (e). In some embodiments, the methods further comprise detecting absence of hybridization of the amplified DNA with a microcarrier having an identifier corresponding to a negative control in (d), wherein the microcarrier with the identifier corresponding to the negative control comprises a probe that does not hybridize with the amplified DNA; and detecting the identifier corresponding to the negative control in (e).

In another aspect, provided herein is a method for detecting the presence of mutations in the ALK, ROS, RET, NTRK1, and cMET genes, the method comprising: (a) isolating RNA from a sample (e.g., obtained from a patient); (b) amplifying (e.g., in vitro) DNA from the isolated RNA by reverse transcription-polymerase chain reaction (RT-PCR), wherein amplifying the DNA comprises: (1) generating cDNA specific for each of the ALK, ROS, RET, NTRK1, and cMET genes from the isolated RNA using a first primer specific for each of the ALK, ROS, RET, NTRK1, and cMET genes, the isolated RNA, and a reverse transcriptase, and (2) amplifying DNA specific for each of the ALK, ROS, RET, NTRK1, and cMET genes by polymerase chain reaction (PCR) using the cDNA generated in (b)(1), a DNA polymerase, the first primer, and a second primer specific for each of the ALK, ROS, RET, NTRK1, and cMET genes that binds to a strand of the cDNA opposite the corresponding first primer and promotes strand extension in a direction opposite that promoted by the corresponding first primer; (c) hybridizing the amplified DNA with at least five probes, said at least five probes comprising one or more probes specific for a mutation in each of the ALK, ROS, RET, NTRK1, and cMET genes, wherein each of said at least five probes is coupled to a microcarrier, and wherein each of the microcarriers comprises an identifier corresponding to the probe coupled thereto; (d) detecting presence or absence of hybridization of the amplified DNA with said at least five probes, wherein hybridization between the amplified DNA and one of the probes indicates the presence of the mutation corresponding to the probe; (e) detecting the identifiers of the microcarriers; and (f) correlating the detected identifiers of the microcarriers with the detected presence or absence of hybridization of the amplified DNA to the corresponding probes of the microcarriers. In some embodiments, the ALK, ROS, RET, NTRK1, and cMET genes are human genes. In some embodiments, one or more of the mutations in the ALK, ROS, RET, and NTRK1 genes comprises a fusion gene. In some embodiments, each of the mutations in the ALK, ROS, RET, and NTRK1 genes comprises a fusion gene.

In some embodiments, the one or more mutations in the ALK gene comprise an EML4-ALK fusion gene. In some embodiments, the first primer is specific for a region of the EML4 locus, and the second primer is specific for a region of the ALK locus. In some embodiments, the second primer is specific for a region of the EML4 locus, and the first primer is specific for a region of the ALK locus. In some embodiments, the one or more mutations in the ALK gene comprise one or more of EML E13:ALK E20, EML E20:ALK E20, and EML E6:ALK E20 EML4-ALK fusion genes. In some embodiments, the one or more mutations in the ALK gene comprise EML E13:ALK E20, EML E20:ALK E20, and EML E6:ALK E20 EML4-ALK fusion genes. In some embodiments, the probes specific for one or more mutations in the ALK gene comprise: (1) a first probe comprising a sequence selected from the group consisting of AAAGGACCTAAAGTGT (SEQ ID NO:161), CCTAAAGTGTACCGC (SEQ ID NO:163), GGGAAAGGACCTAAAG (SEQ ID NO:165), AGTGTACCGCCGGAA (SEQ ID NO:167), and TACCGCCGGAAGCACC (SEQ ID NO:169); (2) a second probe comprising a sequence selected from the group consisting of GACTATGAAATATTGTAC (SEQ ID NO:171), GAAATATTGTACTTGTAC (SEQ ID NO:173), TATTGTACTTGTACCGCC (SEQ ID NO:175), TGTACCGCCGGAAGCAC (SEQ ID NO:177), and CCGCCGGAAGCACCAGGA (SEQ ID NO:179); (3) a third probe comprising a sequence selected from the group consisting of TGTCATCATCAACCAA (SEQ ID NO:181), ATGTCATCATCAACC (SEQ ID NO:183), GTGTACCGCCGGAAGC (SEQ ID NO:185), TCAACCAAGTGTACCG (SEQ ID NO:187), and TACCGCCGGAAGCACCA (SEQ ID NO:189); and (4) a fourth probe comprising a sequence selected from the group consisting of CGAAAAAAACAGCCAA (SEQ ID NO:191), TCGCGAAAAAAACAGC (SEQ ID NO:193), GTGTACCGCCGGAAGC (SEQ ID NO:195), TACCGCCGGAAGCACC (SEQ ID NO:197), and ACAGCCAAGTGTACCG (SEQ ID NO:199); wherein each of the four probes is coupled to a microcarrier with a different identifier. In some embodiments, each of the four probes further comprises eight nucleotides at the 5′ end, and wherein the eight nucleotides at the 5′ end are adenine or thymine nucleotides. In some embodiments, the probes specific for one or more mutations in the ALK gene comprise: (1) a first probe comprising a sequence selected from the group consisting of TTTTTTTTTTAAAGGACCTAAAGTGT (SEQ ID NO:162), TTTTTTTTTTCCTAAAGTGTACCGC (SEQ ID NO:164), TTTTTTTTTTGGGAAAGGACCTAAAG (SEQ ID NO:166), TTTTTTTTTTAGTGTACCGCCGGAA (SEQ ID NO:168), and TTTTTTTTTTTACCGCCGGAAGCACC (SEQ ID NO: 170); (2) a second probe comprising a sequence selected from the group consisting of TTTTTTTTTTTGACTATGAAATATTGTAC (SEQ ID NO:172), TTTTTTTTTTTTGAAATATTGTACTTGTAC (SEQ ID NO:174), TTTTTTTTTTTTTATTGTACTTGTACCGCC (SEQ ID NO:176), TTTTTTTTTTTTTTGTACCGCCGGAAGCAC (SEQ ID NO:178), and TTTTTTTTTTTTCCGCCGGAAGCACCAGGA (SEQ ID NO:180); (3) a third probe comprising a sequence selected from the group consisting of TTTTTTTTTTTTTTTGTCATCATCAACCAA (SEQ ID NO:182), TTTTTTTTTTTTTTATGTCATCATCAACC (SEQ ID NO:184), TTTTTTTTTTTTTTGTGTACCGCCGGAAGC (SEQ ID NO:186), TTTTTTTTTTTTTTTCAACCAAGTGTACCG (SEQ ID NO:188), and TTTTTTTTTTTTTTTACCGCCGGAAGCACCA (SEQ ID NO:190); and (4) a fourth probe comprising a sequence selected from the group consisting of TTTTTTTTTTTTTCGAAAAAAACAGCCAA (SEQ ID NO:192), TTTTTTTTTTTTTTCGCGAAAAAAACAGC (SEQ ID NO:194), TTTTTTTTTTTTTGTGTACCGCCGGAAGC (SEQ ID NO: 196), TTTTTTTTTTTTTTACCGCCGGAAGCACC (SEQ ID NO: 198), and TTTTTTTTTTTTTTACAGCCAAGTGTACCG (SEQ ID NO:200); wherein each of the four probes is coupled to a microcarrier with a different identifier. In some embodiments, the first primer specific for one or more mutations in the ALK gene comprises the sequence AGTTGGGGTTGTAGTCGGTCAT (SEQ ID NO:363) or GAAGCCTCCCTGGATCTCC (SEQ ID NO:364). In some embodiments, the second primer specific for one or more mutations in the ALK gene comprises a sequence selected from the group consisting of TATGGAGCAAAACTACTGTAGAGCC (SEQ ID NO:357), CCAGCTACATCACACACCTTGACT (SEQ ID NO:358), and TAATACCAAAAGTTACCAAAACTGCA (SEQ ID NO:359).

In some embodiments, the one or more mutations in the ROS gene comprise an ROS fusion gene selected from the group consisting of CD74-ROS, and SLC34A2-ROS. In some embodiments, the first primer is specific for a region of the CD74, or SLC34A2, and the second primer is specific for a region of the ROS locus. In some embodiments, the second primer is specific for a region of the CD74, or SLC34A2, locus, and the first primer is specific for a region of the ROS locus. In some embodiments, the first primer is specific for a region of the CD74 or SLC34A2 locus, and the second primer is specific for a region of the ROS locus; or wherein the second primer is specific for a region of the CD74 or SLC34A2 locus, and the first primer is specific for a region of the ROS locus. In some embodiments, the one or more mutations in the ROS gene comprise one or more of CD74 E6:ROS E32, CD74 E6:ROS E34, SLC34A2 E4:ROS E32, and SLC34A2 E4:ROS E34 fusion genes. In some embodiments, the one or more mutations in the ROS gene comprise CD74 E6:ROS E32, CD74 E6:ROS E34, SLC34A2 E4:ROS E32, and SLC34A2 E4:ROS E34 fusion genes. In some embodiments, the probes specific for one or more mutations in the ROS gene comprise: (1) a first probe comprising a sequence selected from the group consisting of ACTGACGCTCCACCGAAA (SEQ ID NO:201), CCACTGACGCTCCACCGA (SEQ ID NO:203), GCTGGAGTCCCAAATAAAC (SEQ ID NO:205), GGAGTCCCAAATAAACCAG (SEQ ID NO:207), and CACCGAAAGCTGGAGTCCC (SEQ ID NO:209); (2) a second probe comprising a sequence selected from the group consisting of CCGAAAGATGATTTT (SEQ ID NO:211), GACGCTCCACCGAAA (SEQ ID NO:213). ACTGACGCTCCACCGA (SEQ ID NO:215), GATGATTTTTGGATA (SEQ ID NO:217), and TGATTTTTGGATACCA (SEQ ID NO:219); (3) a third probe comprising a sequence selected from the group consisting of AGCGCCTTCCAGCTGGTTGGA (SEQ ID NO:221), CTGGTTGGAGCTGGAGTCCC (SEQ ID NO:223), AGTAGCGCCTTCCAGCTGGTTG (SEQ ID NO:225), GCTGGAGTCCCAAATAAACCA (SEQ ID NO:227), and GGAGTCCCAAATAAACCAGG (SEQ ID NO:229); and (4) a fourth probe comprising a sequence selected from the group consisting of GCGCCTTCCAGCTGGTTG (SEQ ID NO:231), GTAGCGCCTTCCAGCTGGT (SEQ ID NO:233), TGGTTGGAGATGATTTTT (SEQ ID NO:235), GATGATTTTTGGATACCAG (SEQ ID NO:237), and TGATTTTTGGATACCA (SEQ ID NO:239); wherein each of the four probes is coupled to a microcarrier with a different identifier. In some embodiments, each of the four probes further comprises eight nucleotides at the 5′ end, and wherein the eight nucleotides at the 5′ end are adenine or thymine nucleotides. In some embodiments, the probes specific for one or more mutations in the ROS gene comprise: (1) a first probe comprising a sequence selected from the group consisting of TTTTTTTTTTTACTGACGCTCCACCGAAA (SEQ ID NO:202), TTTTTTTTTTTCCACTGACGCTCCACCGA (SEQ ID NO:204), TTTTTTTTTTTGCTGGAGTCCCAAATAAAC (SEQ ID NO:206), TTTTTTTTTTTGGAGTCCCAAATAAACCAG (SEQ ID NO:208), and TTTTTTTTTTTCACCGAAAGCTGGAGTCCC (SEQ ID NO:210); (2) a second probe comprising a sequence selected from the group consisting of TTTTTTTTTTTTCCGAAAGATGATTTT (SEQ ID NO:212), TTTTTTTTTTTTGACGCTCCACCGAAA (SEQ ID NO:214), TTTTTTTTTTTTACTGACGCTCCACCGA (SEQ ID NO:216), TTTTTTTTTTTTGATGATTTTTGGATA (SEQ ID NO:218), and TTTTTTTTTTTTTGATTTTTGGATACCA (SEQ ID NO:220); (3) a third probe comprising a sequence selected from the group consisting of TTTTTTTTTTTTAGCGCCTTCCAGCTGGTTGGA (SEQ ID NO:222), TTTTTTTTTTTTCTGGTTGGAGCTGGAGTCCC (SEQ ID NO:224), TTTTTTTTTTTTAGTAGCGCCTTCCAGCTGGTTG (SEQ ID NO:226), TTTTTTTTTTTTGCTGGAGTCCCAAATAAACCA (SEQ ID NO:228), and TTTTTTTTTTTTGGAGTCCCAAATAAACCAGG (SEQ ID NO:230); and (4) a fourth probe comprising a sequence selected from the group consisting of TTTTTTTTTTGCGCCTTCCAGCTGGTTG (SEQ ID NO:232), TTTTTTTTTTGTAGCGCCTTCCAGCTGGT (SEQ ID NO:234), TTTTTTTTTTTGGTTGGAGATGATTTTT (SEQ ID NO:236), TTTTTTTTTTGATGATTTTTGGATACCAG (SEQ ID NO:238), and TTTTTTTTTTTGATTTTTGGATACCA (SEQ ID NO:240); wherein each of the four probes is coupled to a microcarrier with a different identifier. In some embodiments, the first primer specific for one or more mutations in the ROS gene comprises the sequence AATTCAATACATACTATCAGCTTTCTCCCACTGTATTGAA (SEQ ID NO:21) or AATATTTCTGGTACGAGTGGGATTGTAACAACCAGAAATA (SEQ ID NO:22). In some embodiments, the second primer specific for one or more mutations in the ROS gene comprises the sequence GGAGTGCCATCGCTGTTTGAAATGAGCAGGCACT (SEQ ID NO:19) or TACAGCCCTGGATATTCTTAGTAGCGC (SEQ ID NO:20).

In some embodiments, the one or more mutations in the RET gene comprise a RET fusion gene selected from the group consisting of KIF5B-RET. In some embodiments, the first primer is specific for a region of the KIF5B, or CCDC6 locus, and the second primer is specific for a region of the RET locus. In some embodiments, the second primer is specific for a region of the KIF5B, or CCDC6 locus, and the first primer is specific for a region of the RET locus. In some embodiments, the first primer is specific for a region of the KIF5B locus, and the second primer is specific for a region of the RET locus; or wherein the second primer is specific for a region of the KIF5B locus, and the first primer is specific for a region of the RET locus. In some embodiments, the one or more mutations in the RET gene comprise one or more of KIF5B E15:RET E11, KIF5B E15:RET E12, KIF5B E16:RET E12, KIF5B E22:RET E12, KIF5B E23:RET E12, and CCDC6 E1:RET E12 fusion genes. In some embodiments, the one or more mutations in the RET gene comprise KIF5B E15:RET E11, KIF5B E15:RET E12, KIF5B E16:RET E12, KIF5B E22:RET E12, and KIF5B E23:RET E12 fusion genes. In some embodiments, the probes specific for one or more mutations in the RET gene comprise: (1) a first probe comprising a sequence selected from the group consisting of GTGGGAAATAATGATGTAAA (SEQ ID NO:241), CTGTGGGAAATAATGATGTA (SEQ ID NO:243), GATCCACTGTGCGACGAGCT (SEQ ID NO:245), TGATGTAAAGATCCACTGTG (SEQ ID NO:247), and TCCACTGTGCGACGAGCTGT (SEQ ID NO:249); (2) a second probe comprising a sequence selected from the group consisting of TGGGAAATAATGATGTAAA (SEQ ID NO:251), CTGTGGGAAATAATGATGTA (SEQ ID NO:253), GGAGGATCCAAAGTGGGAAT (SEQ ID NO:255), GGATCCAAAGTGGGAATT (SEQ ID NO:257), and ATGATGTAAAGGAGGATCC (SEQ ID NO:259); (3) a third probe comprising a sequence selected from the group consisting of CTTCGTATCTCTCAAGAGGAT (SEQ ID NO:481), GTATCTCTCAAGAGGATCCAA (SEQ ID NO:483), TTCGTATCTCTCAAGAG (SEQ ID NO:485), TCAAGAGGATCCAAA (SEQ ID NO:487), and TCTCTCAAGAGG (SEQ ID NO:489); (4) a fourth probe comprising a sequence selected from the group consisting of GTTAAAAAGGAGGATCCAA (SEQ ID NO:491), ACAAGAGTTAAAAAGGAGGA (SEQ ID NO:493), AAGAGTTAAAAAGGAGGATC (SEQ ID NO:495), AAAAGGAGGATCCAAAG (SEQ ID NO:497), and AAGGAGGATCCAAAGTG (SEQ ID NO:499); and (5) a fifth probe comprising a sequence selected from the group consisting of AAACAGGAGGATCCAAA (SEQ ID NO:501), AAGTGCACAAACAGGAGG (SEQ ID NO:503), GTGCACAAACAGGAGGATC (SEQ ID NO:505), CACAAACAGGAGGAT (SEQ ID NO:507), and AACAGGAGGATCCAAA (SEQ ID NO:509); wherein each of the five probes is coupled to a microcarrier with a different identifier. In some embodiments, each of the four probes further comprises eight nucleotides at the 5′ end, and wherein the eight nucleotides at the 5′ end are adenine or thymine nucleotides. In some embodiments, the probes specific for one or more mutations in the RET gene comprise: (1) a first probe comprising a sequence selected from the group consisting of TTTTTTTTTTGTGGGAAATAATGATGTAAA (SEQ ID NO:242), TTTTTTTTTTCTGTGGGAAATAATGATGTA (SEQ ID NO:244), TTTTTTTTTTGATCCACTGTGCGACGAGCT (SEQ ID NO:246), TTTTTTTTTTTGATGTAAAGATCCACTGTG (SEQ ID NO:248), and TTTTTTTTTTTCCACTGTGCGACGAGCTGT (SEQ ID NO:250); (2) a second probe comprising a sequence selected from the group consisting of TTTTTTTTTTGGGAAATAATGATGTAAA (SEQ ID NO:252), TTTTTTTTTCTGTGGGAAATAATGATGTA (SEQ ID NO:254), TTTTTTTTTGGAGGATCCAAAGTGGGAAT (SEQ ID NO:256), TTTTTTTTTGGATCCAAAGTGGGAATT (SEQ ID NO:258), and TTTTTTTATGATGTAAAGGAGGATCC (SEQ ID NO:260); (3) a third probe comprising a sequence selected from the group consisting of TTTTTTTTTCTTCGTATCTCTCAAGAGGAT (SEQ ID NO:482), TTTTTTTTTGTATCTCTCAAGAGGATCCAA (SEQ ID NO:484), TTTTTTTTTTTCGTATCTCTCAAGAG (SEQ ID NO:486), TTTTTTTTTTCAAGAGGATCCAAA (SEQ ID NO:488), and TTTTTTTTTTCTCTCAAGAGG (SEQ ID NO:490); (4) a fourth probe comprising a sequence selected from the group consisting of TTTTTTTTTGTTAAAAAGGAGGATCCAA (SEQ ID NO:492), TTTTTTTTACAAGAGTTAAAAAGGAGGA (SEQ ID NO:494), TTATTATTAAGAGTTAAAAAGGAGGATC (SEQ ID NO:811), TTTTTTTTAAAAGGAGGATCCAAAG (SEQ ID NO:498), and TTTTTTTTAAGGAGGATCCAAAGTG (SEQ ID NO:500); and (5) a fifth probe comprising a sequence selected from the group consisting of TTTTTTTTAAACAGGAGGATCCAAA (SEQ ID NO:502), TTTTTATTAAGTGCACAAACAGGAGG (SEQ ID NO:504), TATTATTATGTGCACAAACAGGAGGATC (SEQ ID NO:506), TATTTTTTCACAAACAGGAGGAT (SEQ ID NO:508), and TTTTATTTAACAGGAGGATCCAAA (SEQ ID NO:510); wherein each of the five probes is coupled to a microcarrier with a different identifier. In some embodiments, the first primer specific for one or more mutations in the RET gene comprises the sequence GTGATCGCACAGTAGGACAGCGGCTGCGATC (SEQ ID NO:26) or CTCTAGGAGATATCATTCCAAATTCGCCTTCTCCTAG (SEQ ID NO:27). In some embodiments, the second primer specific for one or more mutations in the RET gene comprises a sequence selected from the group consisting of TTTCTGGTGCTATGAGGAAATGACCAACCACCAGA (SEQ ID NO:23), AAGGAGTTAGCAGCATGTCAGC (SEQ ID NO:519), AACTTCAGACTTTACACAACCTGC (SEQ ID NO:520), and ATTGATTCTGATGACACCGGA (SEQ ID NO:521).

In some embodiments, the one or more mutations in the NTRK1 gene comprise a CD74-NTRK1 fusion gene. In some embodiments, the first primer is specific for a region of the CD74 locus, and the second primer is specific for a region of the NTRK1 locus. In some embodiments, the second primer is specific for a region of the CD74 locus, and the first primer is specific for a region of the NTRK1 locus. In some embodiments, the one or more mutations in the NTRK1 gene comprise a CD74 E8:NTRK1 E12 fusion gene. In some embodiments, the probe specific for one or more mutations in the NTRK1 gene comprises a sequence selected from the group consisting of CAGGATCTGGGCCCAGACA (SEQ ID NO:261), GATCTGGGCCCAGACACTA (SEQ ID NO:263), CCAGACACTAACAGCACAT (SEQ ID NO:265), GGGCCCAGACACTAACAGC (SEQ ID NO:267), and CTAACAGCACATCTGGAGA (SEQ ID NO:269). In some embodiments, the probe specific for one or more mutations in the NTRK1 gene further comprises eight nucleotides at the 5′ end, and wherein the eight nucleotides at the 5′ end are adenine or thymine nucleotides. In some embodiments, the probe specific for one or more mutations in the NTRK1 gene comprises a sequence selected from the group consisting of TTTTTTTTTTACAGGATCTGGGCCCAGACA (SEQ ID NO:262), TTTTTTTTTTAGATCTGGGCCCAGACACTA (SEQ ID NO:264), TTTTTTTTTTACCAGACACTAACAGCACAT (SEQ ID NO:266), TTTTTTTTTTAGGGCCCAGACACTAACAGC (SEQ ID NO:268), and TTTTTTTTTTACTAACAGCACATCTGGAGA (SEQ ID NO:270). In some embodiments, the first primer specific for one or more mutations in the NTRK1 gene comprises the sequence GGACGAAAATCCAGACCCCAAAAGGTGTTTCGT (SEQ ID NO:32). In some embodiments, the second primer specific for one or more mutations in the NTRK1 gene comprises the sequence AGAAGACGTGACAGGAACTGGAGGACCCGTCTT (SEQ ID NO:30).

In some embodiments, the one or more mutations in the cMET gene results in exon skipping. In some embodiments, the one or more mutations in the cMET gene results in skipping of exon 14. In some embodiments, the probe specific for one or more mutations in the cMET gene comprises a sequence selected from the group consisting of AGAAAGCAAATTAAAGAT (SEQ ID NO:271), AGCAAATTAAAGATCAG (SEQ ID NO:273), AAATTAAAGATCAGTTTC (SEQ ID NO:275), AGATCAGTTTCCTAATTC (SEQ ID NO:277), and AAGATCAGTTTCCTAATT (SEQ ID NO:279). In some embodiments, the probe specific for one or more mutations in the cMET gene further comprises eight nucleotides at the 5′ end, and wherein the eight nucleotides at the 5′ end are adenine or thymine nucleotides. In some embodiments, the probe specific for one or more mutations in the cMET gene comprises a sequence selected from the group consisting of TTTTTTTTTTAGAAAGCAAATTAAAGAT (SEQ ID NO:272), TTTTTTTTTTAGCAAATTAAAGATCAG (SEQ ID NO:274), TTTTTTTTTTAAATTAAAGATCAGTTTC (SEQ ID NO:276), TTTTTTTTTTAGATCAGTTTCCTAATTC (SEQ ID NO:278), and TTTTTTTTTTAAGATCAGTTTCCTAATT (SEQ ID NO:280). In some embodiments, the first primer specific for one or more mutations in the cMET gene comprises the sequence GACAGTATTTTGCAGTAATGGACTGGATATATCAGA (SEQ ID NO:29). In some embodiments, the second primer specific for one or more mutations in the cMET gene comprises the sequence GAATTTCACAGGATTGATTGCTGGTGTTGTCTC (SEQ ID NO:28).

In some embodiments according to any of the embodiments described herein, the sample is a blood, serum, or plasma sample. In some embodiments, isolating RNA from the sample in (a) comprises isolating RNA from one or more of tumor-conditioned platelets, tumor exosomes, and circulating tumor cells (CTCs). In some embodiments, the methods further comprise amplifying a positive control DNA sequence from the isolated RNA by reverse transcription-polymerase chain reaction (RT-PCR) in (b), wherein amplifying the positive control DNA sequence comprises: (1) generating cDNA specific for the positive control sequence from the isolated RNA using a first primer specific for the positive control sequence, the isolated RNA, and a reverse transcriptase, and (2) amplifying DNA specific for the positive control sequence by polymerase chain reaction (PCR) using the cDNA specific for the positive control sequence generated in (1), a DNA polymerase, the first primer, and a second primer specific for the positive control sequence that binds to a strand of the cDNA opposite the corresponding first primer and promotes strand extension in a direction opposite that promoted by the corresponding first primer; hybridizing the amplified positive control gene sequence with a probe specific for the positive control gene sequence in (c), wherein the probe specific for the positive control gene sequence is coupled to a microcarrier with an identifier corresponding to a positive control; detecting presence or absence of hybridization of the amplified positive control DNA sequence with the probe specific for the positive control gene sequence in (d); and detecting the identifier corresponding to the positive control in (e). In some embodiments, the methods further comprise detecting absence of hybridization of the amplified DNA with a microcarrier having an identifier corresponding to a negative control in (d), wherein the microcarrier with the identifier corresponding to the negative control comprises a probe that does not hybridize with the amplified DNA; and detecting the identifier corresponding to the negative control in (e).

In another aspect, provided herein is a method for detecting the presence of mutations in the genes, the method comprising: (a) isolating DNA and RNA from a sample; (b) amplifying the isolated DNA by polymerase chain reaction (PCR) using primer pairs specific for the loci of one or more DNA mutations in each of the KRAS, NRAS, PIK3CA, BRAF, EGFR, AKT1, MEK1, and HER2 genes; (c) amplifying DNA from the isolated RNA by reverse transcription-polymerase chain reaction (RT-PCR), wherein amplifying the DNA from the isolated RNA comprises: (1) generating cDNA specific for each of the ALK, ROS, RET, NTRK1, and cMET genes from the isolated RNA using a first primer specific for each of the ALK, ROS, RET, NTRK1, and cMET genes, the isolated RNA, and a reverse transcriptase, and (2) amplifying DNA specific for each of the ALK, ROS, RET, NTRK1, and cMET genes by polymerase chain reaction (PCR) using the cDNA generated in (c)(1), a DNA polymerase, the first primer, and a second primer specific for each of the ALK, ROS, RET, NTRK1, and cMET genes that binds to a strand of the cDNA opposite the corresponding first primer and promotes strand extension in a direction opposite that promoted by the corresponding first primer; (d) hybridizing the DNA amplified by PCR in (b) with at least seven probes, said at least seven probes comprising one or more probes specific for a mutation in each of the KRAS, NRAS, PIK3CA, BRAF, EGFR, AKT1, MEK1, and HER2 genes, wherein each of said at least seven probes is coupled to a microcarrier, and wherein each of the microcarriers comprises an identifier corresponding to the probe coupled thereto; (e) detecting presence or absence of hybridization of the DNA amplified by PCR in (b) with said at least seven probes, wherein hybridization between the amplified DNA and one of the probes indicates the presence of the mutation corresponding to the probe; (f) hybridizing the DNA amplified by RT-PCR in (c) with at least five probes, said at least five probes comprising one or more probes specific for a mutation in each of the ALK, ROS, RET, NTRK1, and cMET genes, wherein each of said at least five probes is coupled to a microcarrier, and wherein each of the microcarriers comprises an identifier corresponding to the probe coupled thereto; (g) detecting presence or absence of hybridization of the DNA amplified by RT-PCR in (c) with said at least five probes, wherein hybridization between the amplified DNA and one of the probes indicates the presence of the mutation corresponding to the probe; (h) detecting the identifiers of the microcarriers; and (i) correlating the detected identifiers of the microcarriers with the presence or absence of hybridization of the amplified DNA to the corresponding probes of the microcarriers detected in (e) and (g). In some embodiments, (a) comprises: isolating total RNA-rich plasma (TRRP) by centrifuging the sample, wherein the sample comprises whole blood or plasma; subjecting the TRRP to one or more centrifugation steps to generate an RNA fraction and a cell-free DNA (cfDNA) fraction, wherein the RNA fraction comprises one or more of: platelets, white blood cells, exosomes, circulating tumor cells, and free RNA; isolating DNA from the cfDNA fraction; and isolating RNA from the RNA fraction.

In some embodiments according to any of the embodiments described herein, each of the primer pairs comprises a primer coupled to a detection reagent. In some embodiments, the detection reagent comprises a fluorescent detection reagent, and wherein detecting the presence or absence of hybridization of the amplified DNA with said probes in (d) comprises fluorescence imaging of the fluorescent detection reagent. In some embodiments, the detection reagent comprises biotin, and wherein detecting the presence or absence of hybridization of the amplified DNA with said probes in step (d) comprises: (1) after hybridization in (c), contacting the microcarriers with streptavidin conjugated to a signal-emitting entity; and (2) detecting a signal from the signal-emitting entity in association with the microcarriers. In some embodiments, the signal-emitting entity comprises phycoerythrin (PE). In some embodiments, detecting the identifiers of the microcarriers in (e) comprises bright field imaging of the identifiers. In some embodiments, the identifiers of the microcarriers comprise digital barcodes. In some embodiments, each of the microcarriers comprises: (i) a first photopolymer layer; (ii) a second photopolymer layer; and (iii) an intermediate layer between the first layer and the second layer, the intermediate layer having an encoded pattern representing the identifier defined thereon, wherein the intermediate layer is partially substantially transmissive and partially substantially opaque to light, representing a code corresponding to the microcarrier, wherein the outermost surface of the microcarrier comprises a photoresist photopolymer, and said photoresist photopolymer is functionalized with the probe specific for the DNA mutation, and wherein said microcarrier has about the same density as water. In some embodiments, the identifiers of the microcarriers comprise analog codes. In some embodiments, each of the microcarriers comprises: (i) a substantially transparent polymer layer having a first surface and a second surface, the first and the second surfaces being parallel to each other; (ii) a substantially non-transparent layer that constitutes a two-dimensional shape, wherein the substantially non-transparent layer is affixed to the first surface of the substantially transparent polymer layer and encloses a center portion of the substantially transparent polymer layer, wherein the two-dimensional shape of the substantially non-transparent layer represents an analog code, and wherein the analog code corresponds to the identifier; and (iii) the probe specific for the mutation, wherein the probe is coupled to at least one of the first surface and the second surface of the substantially transparent polymer layer in at least the center portion of the substantially transparent polymer layer. In some embodiments, each of the microcarriers further comprises an orientation indicator for orienting the analog code of the substantially non-transparent polymer layer. In some embodiments, the polymer of the substantially transparent polymer layer comprises an epoxy-based polymer. In some embodiments, the epoxy-based polymer is SU-8.

In another aspect, provided herein is a kit comprising at least seven microcarriers, wherein each of said at least seven microcarriers comprises: (i) a probe coupled to the microcarrier, wherein the probe is specific for a DNA mutation in the KRAS, PIK3CA, BRAF, EGFR, AKT1, MEK1, or HER2 gene; and (ii) an identifier corresponding to the probe coupled thereto; wherein the kit comprises at least one microcarrier comprising a probe specific for a DNA mutation in the KRAS gene, at least one microcarrier comprising a probe specific for a DNA mutation in the PIK3CA gene, at least one microcarrier comprising a probe specific for a DNA mutation in the BRAF gene, at least one microcarrier comprising a probe specific for a DNA mutation in the EGFR gene, at least one microcarrier comprising a probe specific for a DNA mutation in the AKT1 gene, at least one microcarrier comprising a probe specific for a DNA mutation in the MEK1 gene, and at least one microcarrier comprising a probe specific for a DNA mutation in the HER2 gene; and wherein the KRAS, NRAS, PIK3CA, BRAF, EGFR, AKT1, MEK1, and HER2 genes are human genes.

In some embodiments, the kit further comprises at least seven blocking nucleic acids, wherein each of said at least seven blocking nucleic acids hybridizes with a wild-type DNA locus corresponding with one of the DNA mutations in the KRAS, PIK3CA, BRAF, EGFR, AKT1, MEK1, or HER2 genes and prevents amplification of the wild-type DNA locus. In some embodiments, each of said at least seven blocking nucleic acids comprises: a single-stranded oligonucleotide that hybridizes with the corresponding wild-type DNA locus; and a 3′ terminal moiety that blocks extension from the single-stranded oligonucleotide. In some embodiments, the 3′ terminal moiety comprises one or more inverted deoxythynmidines. In some embodiments, each of said at least seven blocking nucleic acids comprises one or more modified nucleotides selected from the group consisting of locked nucleic acids (LNAs), peptide nucleic acids (PNAs), hexose nucleic acids (HNAs), threose nucleic acids (TNAs), glycol nucleic acids (GNAs), and cyclohexenyl nucleic acids (CeNAs).

In some embodiments, the DNA mutation in the KRAS gene comprises one or more DNA mutations encoding a G12D, G12V, or G12C mutated KRAS protein. In some embodiments, the DNA mutation in the KRAS gene comprises DNA mutations encoding G12D, G12V, and G12C mutated KRAS proteins. In some embodiments, the probes specific for the DNA mutation in the KRAS gene comprise: (1) a first probe comprising a sequence selected from the group consisting of TAGTTGGAGCT (SEQ ID NO:38), TGTGGTAGTTG (SEQ ID NO:40), TGATGGCGTAG (SEQ ID NO:42), TGGAGCTGATGGC (SEQ ID NO:44), and GCGTAGGCAAG (SEQ ID NO:46); (2) a second probe comprising a sequence selected from the group consisting of CTGTTGGCGTAGG (SEQ ID NO:48), GTAGTTGGAGCTG (SEQ ID NO:50), TGGAGCTGTTGGC (SEQ ID NO:52), TTGTGGTAGTTGG (SEQ ID NO:54), and GGCGTAGGCAAGA (SEQ ID NO:56); and (3) a third probe comprising a sequence selected from the group consisting of TAGTTGGAGCTT (SEQ ID NO:58), GCGTAGGCAAGA (SEQ ID NO:60), GGAGCTTGTGGC (SEQ ID NO: 62), TTGTGGCGTAGG (SEQ ID NO:64), and TGTGGTAGTTGG (SEQ ID NO:66); wherein each of the three probes is coupled to a microcarrier with a different identifier. In some embodiments, each of the three probes further comprises eight nucleotides at the 5′ end, and wherein the eight nucleotides at the 5′ end are adenine or thymine nucleotides. In some embodiments, the probes specific for the DNA mutation in the KRAS gene comprise: (1) a first probe comprising a sequence selected from the group consisting of TTTTTTTTTTTTAATAGTTGGAGCT (SEQ ID NO:39), TTTTTTTTTTTTAATGTGGTAGTTG (SEQ ID NO:41), TTTTTTTTTTTTAATGATGGCGTAG (SEQ ID NO: 43), TTTTTTTTTTTATGGAGCTGATGGC (SEQ ID NO: 45), and TTTTTTTTTTTTAAGCGTAGGCAAG (SEQ ID NO:47); (2) a second probe comprising a sequence selected from the group consisting of TTTTTTTTTTTACTGTTGGCGTAGG (SEQ ID NO:49), TTTTTTTTTTTAGTAGTTGGAGCTG (SEQ ID NO:51), TTTTTTTTTTTATGGAGCTGTTGGC (SEQ ID NO:53), TTTTTTTTTTTATTGTGGTAGTTGG (SEQ ID NO:55), and TTTTTTTTTTTAGGCGTAGGCAAGA (SEQ ID NO:57); and (3) a third probe comprising a sequence selected from the group consisting of TTTTTTTTTTTAATAGTTGGAGCTT (SEQ ID NO:59), TTTTTTTTTTTAAGCGTAGGCAAGA (SEQ ID NO:61), TTTTTTTTTTTAAGGAGCTTGTGGC (SEQ ID NO: 63), TTTTTTTTTTTAATTGTGGCGTAGG (SEQ ID NO: 65), and TTTTTTTTTTTAATGTGGTAGTTGG (SEQ ID NO:67); wherein each of the three probes is coupled to a microcarrier with a different identifier. In some embodiments, the kit further comprises a primer pair comprising the sequences GTACTGGTGGAGTATTTGATAGTG (SEQ ID NO:1) and CGTCAAGGCACTCTTGCCTAC (SEQ ID NO:2). In some embodiments, the kit further comprises a blocking nucleic acid that hybridizes with a wild-type KRAS DNA locus corresponding with the KRAS DNA mutation and prevents amplification of the wild-type KRAS DNA locus, and wherein the blocking nucleic acid comprises the sequence TACGCCACCAGCT(invdT)ₙ, wherein n is 1, 2, or 3 (SEQ ID NO:281); TTGGAGCTGGTGGCGTA(invdT)ₙ, wherein n is 1, 2, or 3 (SEQ ID NO:282); GCTGGTGGCGTAGGCA(invdT)ₙ, wherein n is 1, 2, or 3 (SEQ ID NO:283); GCTGGTGGCGTAGGC(invdT)ₙ, wherein n is 1, 2, or 3 (SEQ ID NO:284); or TTGGAGCTGGTGGCGT(invdT)ₙ, wherein n is 1, 2, or 3 (SEQ ID NO:285); with italicized nucleic acids representing locked nucleic acids. In some embodiments, the DNA mutation in the PIK3CA gene comprises one or more DNA mutations encoding an E542K or E545K mutated PIK3CA protein. In some embodiments, the DNA mutation in the PIK3CA gene comprises DNA mutations encoding E542K and E545K mutated PIK3CA proteins. In some embodiments, the probes specific for the DNA mutation in the PIK3CA gene comprise: (1) a first probe comprising a sequence selected from the group consisting of GCTCAGTGATTTTAG (SEQ ID NO:87), TGCTCAGTGATTTT (SEQ ID NO: 89), GCTCAGTGATTTTAG (SEQ ID NO:91), CCTGCTCAGTGATTTTA (SEQ ID NO:93), and CTCAGTGATTTTAGA (SEQ ID NO:95); and (2) a second probe comprising a sequence selected from the group consisting of TTCTCCTGCTTA (SEQ ID NO:97), CTCCTGCTTAGT (SEQ ID NO:99), TCTCCTGCTTAG (SEQ ID NO: 101), TCCTGCTTAGTG (SEQ ID NO:103), and CTCCTGCTTAGTGA (SEQ ID NO:105); wherein each of the two probes is coupled to a microcarrier with a different identifier. In some embodiments, each of the two probes further comprises eight nucleotides at the 5′ end, and wherein the eight nucleotides at the 5′ end are adenine or thymine nucleotides. In some embodiments, the probes specific for the DNA mutation in the PIK3CA gene comprise: (1) a first probe comprising a sequence selected from the group consisting of TTTTTTTTTAGCTCAGTGATTTTAG (SEQ ID NO:88), TTTTTTTTTTGCTCAGTGATTTT (SEQ ID NO:90), TTTTTTTTTAGCTCAGTGATTTTAG (SEQ ID NO:92), TTTTTTTCCTGCTCAGTGATTTTA (SEQ ID NO: 94), and TTTTTTTTTTTCTCAGTGATTTTAGA (SEQ ID NO:96); and (2) a second probe comprising a sequence selected from the group consisting of TTTTTTTTTTTTTCTCCTGCTTA (SEQ ID NO:98), TTTTTTTTTTTTTCTCCTGCTTAGT (SEQ ID NO:100), TTTTTTTTTTTATCTCCTGCTTAG (SEQ ID NO:102), TTTTTTTTTTTTTTTCCTGCTTAGTG (SEQ ID NO:104), and TTTTTTTTTTTTTCTCCTGCTTAGTGA (SEQ ID NO:106); wherein each of the three probes is coupled to a microcarrier with a different identifier. In some embodiments, the kit further comprises a primer pair comprising the sequences CAATTTCTACAAGAGATCCTCTCTCT (SEQ ID NO:5) and CTCCATTTTAGCACTTACCTGTGAC (SEQ ID NO:6). In some embodiments, the kit further comprises a blocking nucleic acid that hybridizes with a wild-type PIK3CA DNA locus corresponding with the PIK3CA DNA mutation and prevents amplification of the wild-type PIK3CA DNA locus, and the blocking nucleic acid comprises the sequence CTGAAATCACTGAGCAGG(invdT)ₙ, wherein n is 1, 2, or 3 (SEQ ID NO:291); TCTCTGAAATCACTGAGCAGG(invdT)ₙ, wherein n is 1, 2, or 3 (SEQ ID NO:292); TCTCTGAAATCACTGAGCAGG(invdT)ₙ, wherein n is 1, 2, or 3 (SEQ ID NO:293); TCTCTGAAATCACTGAGCAGG(invdT)ₙ, wherein n is 1, 2, or 3 (SEQ ID NO:294); or TCTCTGAATTCACTGAGCAGG(invdT)ₙ, wherein n is 1, 2, or 3 (SEQ ID NO:295); with italicized nucleic acids representing locked nucleic acids. In some embodiments, the DNA mutation in the PIK3CA gene comprises a DNA mutation encoding an H1047R mutated PIK3CA protein. In some embodiments, the probe specific for the DNA mutation in the PIK3CA gene comprises a sequence selected from the group consisting of GATGCACGTCATG (SEQ ID NO:107), TGAATGATGCACG (SEQ ID NO:109), TGATGCACGTC (SEQ ID NO:111), AATGATGCACGTCA (SEQ ID NO:113), and AATGATGCACGTC (SEQ ID NO:115). In some embodiments, the probe specific for the DNA mutation in the PIK3CA gene comprises a sequence selected from the group consisting of TTTTTTTTTTTTTTTGATGCACGTCATG (SEQ ID NO:108), TTTTTTTTTTTGAATGATGCACG (SEQ ID NO:110), TTTTTTTTTTTTTGATGCACGTC (SEQ ID NO:112), TTTTTTTTTTTTAATGATGCACGTCA (SEQ ID NO:114), and TTTTTTTTTTTTAATGATGCACGTC (SEQ ID NO:116). In some embodiments, the probe further comprises eight nucleotides at the 5′ end, and wherein the eight nucleotides at the 5′ end are adenine or thymine nucleotides. In some embodiments, the kit further comprises a primer pair comprising the sequences ACCCTAGCCTTAGATAAAACTGAGC (SEQ ID NO:7) and TTTGTTGTCCAGCCACCATGA (SEQ ID NO:8). In some embodiments, the kit further comprises a blocking nucleic acid that hybridizes with a wild-type PIK3CA DNA locus corresponding with the PIK3CA DNA mutation and prevents amplification of the wild-type PIK3CA DNA locus, and wherein the blocking nucleic acid comprises the sequence CACCATGATGTGCAT(invdT)ₙ, wherein n is 1, 2, or 3 (SEQ ID NO:296); CCACCATGATGTGCAT(invdT)ₙ, wherein n is 1, 2, or 3 (SEQ ID NO:297); CACCATGATGTGCAT(invdT)ₙ, wherein n is 1, 2, or 3 (SEQ ID NO:298); CCACCATGATGTGCATCA(invdT)ₙ, wherein n is 1, 2, or 3 (SEQ ID NO:299); or CATGATGTGCA(invdT)ₙ, wherein n is 1, 2, or 3 (SEQ ID NO:300); with italicized nucleic acids representing locked nucleic acids. In some embodiments, the DNA mutation in the BRAF gene comprises a DNA mutation encoding a V600E mutated BRAF protein. In some embodiments, the probe specific for the DNA mutation in the BRAF gene comprises a sequence selected from the group consisting of TTTGGTCTAGCTACAGA (SEQ ID NO: 79), CTACAGAGAAATCTCGA (SEQ ID NO:81), GTGATTTTGGTCTAGCT (SEQ ID NO:83), and TCTAGCTACAGAGAAAT (SEQ ID NO:85). In some embodiments, the probe specific for one or more DNA mutations in the BRAF gene further comprises eight nucleotides at the 5′ end, and wherein the eight nucleotides at the 5′ end are adenine or thymine nucleotides. In some embodiments, the probe specific for the DNA mutation in the BRAF gene comprises a sequence selected from the group consisting of TTTTTTAATTGAGAAATCTCGATGGAG (SEQ ID NO:78), TTTTTTAATTTTTGGTCTAGCTACAGA (SEQ ID NO:80), TTTTTTAATTCTACAGAGAAATCTCGA (SEQ ID NO:82), TTTTTTAATTGTGATTTTGGTCTAGCT (SEQ ID NO:84), and TTTTTTAATTTCTAGCTACAGAGAAAT (SEQ ID NO:86). In some embodiments, the kit further comprises a primer pair comprising the sequences ATAGCCTCAATTCTTACCATCCACAAAATG (SEQ ID NO:9) and CAGATATATTTCTTCATGAAGACCTCACAGTAA (SEQ ID NO:10). In some embodiments, the kit further comprises a blocking nucleic acid that hybridizes with a wild-type BRAF DNA locus corresponding with the BRAF DNA mutation and prevents amplification of the wild-type BRAF DNA locus, and wherein the blocking nucleic acid comprises the sequence GAGATTTCACTGTAGC(invdT)ₙ, wherein n is 1, 2, or 3 (SEQ ID NO:301); GAGATTTCACTGTAGC(invdT)ₙ, wherein n is 1, 2, or 3 (SEQ ID NO:302); GAGATTTCACTGTAGC(invdT)ₙ, wherein n is 1, 2, or 3 (SEQ ID NO:303); GAGATTCACTGTAGC(invdT)ₙ, wherein n is 1, 2, or 3 (SEQ ID NO:304); or GAGATTTCACTGTAGC(invdT)ₙ, wherein n is 1, 2, or 3 (SEQ ID NO:305); with italicized nucleic acids representing locked nucleic acids. In some embodiments, the probe specific for the DNA mutation in the EGFR gene comprises a sequence selected from the group consisting of TCAAAGTGCTGGCCTC (SEQ ID NO:117), AGATCAAAGTGCTGGCCTCCG (SEQ ID NO:119), AAAGTGCTGGCCT (SEQ ID NO:121), AGTGCTGGCCT (SEQ ID NO:123), and AAGTGCTGGCCTC (SEQ ID NO:125). In some embodiments, the probe specific for the DNA mutation in the EGFR gene further comprises eight nucleotides at the 5′ end, and wherein the eight nucleotides at the 5′ end are adenine or thymine nucleotides. In some embodiments, the probe specific for the DNA mutation in the EGFR gene comprises a sequence selected from the group consisting of TTTTTTTTTTCAAAGTGCTGGCCTC (SEQ ID NO:118), TTTTTTAGATCAAAGTGCTGGCCTCCG (SEQ ID NO:120), TTTTTTTTTTTAAAGTGCTGGCCT (SEQ ID NO: 122), TTTTTTTTTTTTTAGTGCTGGCCT (SEQ ID NO:124), and TTTTTTTTTTTTAAGTGCTGGCCTC (SEQ ID NO:126). In some embodiments, the kit further comprises a primer pair comprising the sequences CTTGTGGAGCCTCTTACACCC (SEQ ID NO:11) and TGCCGAACGCACCGGA (SEQ ID NO:12). In some embodiments, the kit further comprises a blocking nucleic acid that hybridizes with a wild-type EGFR DNA locus corresponding with the EGFR DNA mutation and prevents amplification of the wild-type EGFR DNA locus, and wherein the blocking nucleic acid comprises the sequenceCGGAGCCCAGCACTTTGA (invdT)ₙ, wherein n is 1, 2, or 3 (SEQ ID NO:306); CGCACCGGAGCCCAGCACT (invdT)ₙ, wherein n is 1, 2, or 3 (SEQ ID NO:307); GAGCCCAGCAC (invdT)ₙ, wherein n is 1, 2, or 3 (SEQ ID NO:308); CGCACCGGAGCCCAGCAC (invdT)ₙ, wherein n is 1, 2, or 3 (SEQ ID NO:309); or CGCACCGGAGCCCAGCACTTA (invdT)ₙ, wherein n is 1, 2, or 3 (SEQ ID NO:310); with italicized nucleic acids representing locked nucleic acids. In some embodiments, the DNA mutation in the EGFR gene comprises a DNA mutation encoding an E746_A750del mutated EGFR protein. In some embodiments, the probe specific for the DNA mutation in the EGFR gene comprises: (1) a first probe comprising a sequence selected from the group consisting of AATCAAAACATCTCCGAAAG (SEQ ID NO:128), CAAAACATCTCCG (SEQ ID NO:130), AACATCTCCG (SEQ ID NO:132), and AAACATCTCCGAAAGCC (SEQ ID NO:134); and (2) a second probe comprising a sequence selected from the group consisting of AATCAAGACATCTCCGA (SEQ ID NO:136), GCAATCAAGACATCTCCGA (SEQ ID NO:138), AATCAAGACATCTC (SEQ ID NO:140), AATCAAGACATCTCCGAAAGC (SEQ ID NO:142), and CAAGACATCTCCGA (SEQ ID NO:144); wherein each of the two probes is coupled to a microcarrier with a different identifier. In some embodiments, each of the two probes further comprises eight nucleotides at the 5′ end, and wherein the eight nucleotides at the 5′ end are adenine or thymine nucleotides. In some embodiments, the probe specific for the DNA mutation in the EGFR gene comprises: (1) a first probe comprising a sequence selected from the group consisting of TTTTTTTTTAATCAAAACATCTCCG (SEQ ID NO:127), TTTTTTTTTAATCAAAACATCTCCGAAAG (SEQ ID NO:129), TTTTTTTTTTTACAAAACATCTCCG (SEQ ID NO:131), TTTTTTTTTTTTTTTAACATCTCCG (SEQ ID NO:133), and TTTTTTTTTTTTTTAAACATCTCCGAAAGCC (SEQ ID NO:135); and (2) a second probe comprising a sequence selected from the group consisting of TTTTTTTTAATCAAGACATCTCCGA (SEQ ID NO:137), TTTTTTGCAATCAAGACATCTCCGA (SEQ ID NO: 139), TTTTTTTTAATCAAGACATCTC (SEQ ID NO:141), TTTTTTTTAATCAAGACATCTCCGAAAGC (SEQ ID NO:143), and TTTTTTTTTTTCAAGACATCTCCGA (SEQ ID NO:145); wherein each of the two probes is coupled to a microcarrier with a different identifier. In some embodiments, the kit further comprises a primer pair comprising the sequences GCCAGTTAACGTCTTCCTTCTC (SEQ ID NO:13) and ATCGAGGATTTCCTTGTTGGCTT (SEQ ID NO:14). In some embodiments, the kit further comprises a blocking nucleic acid that hybridizes with a wild-type EGFR DNA locus corresponding with the EGFR DNA mutation and prevents amplification of the wild-type EGFR DNA locus, and wherein the blocking nucleic acid comprises the sequence CGGAGATGTTGCTTCTCTTAATTCC(invdT)ₙ, wherein n is 1, 2, or 3 (SEQ ID NO:311); CGGAGATGTTGCTTCTCT(invdT)ₙ, wherein n is 1, 2, or 3 (SEQ ID NO: 312); GTTGCTTCTCTTAATTCC(invdT)ₙ, wherein n is 1, 2, or 3 (SEQ ID NO: 313); ATGTTGCTTCTCT(invdT)ₙ, wherein n is 1, 2, or 3 (SEQ ID NO:314); or TTGCTTCTCTTA(invdT)ₙ, wherein n is 1, 2, or 3 (SEQ ID NO:315); with italicized nucleic acids representing locked nucleic acids. In some embodiments, the DNA mutation in the EGFR gene comprises one or more DNA mutations encoding a T790M, C797S, S768I, V769_D770insASV, H773_V774insH, D770_N771insG, or D770_N771insSVD mutated EGFR protein. In some embodiments, the DNA mutation in the EGFR gene comprises DNA mutations encoding T790M, C797S, S768I, V769_D770insASV, H773_V774insH, D770_N771insG, and D770_N771insSVD mutated EGFR proteins. In some embodiments, the probe specific for the DNA mutation in the EGFR gene comprises: (1) a first probe comprising a sequence selected from the group consisting of GAGATGCATGATGA (SEQ ID NO:146), TGAGATGCATGATGAG (SEQ ID NO:147), ATGAGATGCATGATGAG (SEQ ID NO:148), TGAGCTGCATGATGA (SEQ ID NO:149), and CATGAGATGCATGATGA (SEQ ID NO: 150); (2) a second probe comprising a sequence selected from the group consisting of CCAGGAGGCTGCCG (SEQ ID NO:461), CAGGAGGCTGCCGA (SEQ ID NO:463), TCCAGGAGGCTGCC (SEQ ID NO:465), CCAGGAGGCTGCC (SEQ ID NO:467), and CAGGAGGCTGCC (SEQ ID NO:469); (3) a third probe comprising a sequence selected from the group consisting of CCAGGAGGGAGCC (SEQ ID NO:471), CCAGGAGGGAGCCG (SEQ ID NO:473), TCCAGGAGGGAGCC (SEQ ID NO:475), CAGGAGGGAGCCG (SEQ ID NO:477), and CAGGAGGGAGCCGA (SEQ ID NO:479); (4) a fourth probe comprising a sequence selected from the group consisting of ATGGCCATCTTGG (SEQ ID NO:421), GGCCATCTTGGA (SEQ ID NO:423), GATGGCCATCTTG (SEQ ID NO:425), TGATGGCCATCTTG (SEQ ID NO:427), and TGGCCATCTTGG (SEQ ID NO: 429); (5) a fifth probe comprising a sequence selected from the group consisting of GTGATGGCCGG (SEQ ID NO:431), TGATGGCCGGCG (SEQ ID NO:433), GTGATGGCCGGCGT (SEQ ID NO:435), GATGGCCGGCGT (SEQ ID NO:437), and GATGGCCCGCGTG (SEQ ID NO:439); (6) a sixth probe comprising a sequence selected from the group consisting of AACCCCCATCACGT (SEQ ID NO:441), GACAACCCCCATCACG (SEQ ID NO:443), CGTGGACAACCCCCATCA (SEQ ID NO:445), CCCATCACGTGT (SEQ ID NO:447), and TGGACAACCCCCATCAC (SEQ ID NO:449); and (7) a seventh probe comprising a sequence selected from the group consisting of GCCAGCGTGGACGG (SEQ ID NO:451), CGTGGACGGTAACC (SEQ ID NO:453), GACGGTAACCCCC (SEQ ID NO:455), CCAGCGTGGACGGT (SEQ ID NO:457), and GCCAGCGTGGACGGTA (SEQ ID NO:459); wherein each of the seven probes is coupled to a microcarrier with a different identifier. In some embodiments, each of the seven probes specific for the DNA mutation in the EGFR gene further comprises eight nucleotides at the 5′ end, and wherein the eight nucleotides at the 5′ end are adenine or thymine nucleotides. In some embodiments, the probe specific for the DNA mutation in the EGFR gene comprises: (1) a first probe comprising a sequence selected from the group consisting of TTTTTTTTTTTGAGATGCATGATGA (SEQ ID NO:352), TTTTTTTTTTGAGATGCATGATGAG (SEQ ID NO:353), TTTTTTTTATGAGATGCATGATGAG (SEQ ID NO:354), TTTTTTTTTTTGAGCTGCATGATGA (SEQ ID NO:355), and TTTTTTTTCATGAGATGCATGATGA (SEQ ID NO:356); (2) a second probe comprising a sequence selected from the group consisting of TTTTTTTTTTTACCAGGAGGCTGCCG (SEQ ID NO:462), TTTTTTTTTTTACAGGAGGCTGCCGA (SEQ ID NO:464), TTTTTTTTTTTATCCAGGAGGCTGCC (SEQ ID NO:466), TTTTTTTTTTTACCAGGAGGCTGCC (SEQ ID NO:468), and TTTTTTTTTTTACAGGAGGCTGCC (SEQ ID NO:470); (3) a third probe comprising a sequence selected from the group consisting of TTTTTTTTTTTACCAGGAGGGAGCC (SEQ ID NO:472), TTTTTTTTTTTACCAGGAGGGAGCCG (SEQ ID NO:474), TTTTTTTTTTTATCCAGGAGGGAGCC (SEQ ID NO:476), TTTTTTTTTTTACAGGAGGGAGCCG (SEQ ID NO:478), and TTTTTTTTTTTACAGGAGGGAGCCGA (SEQ ID NO:480); (4) a fourth probe comprising a sequence selected from the group consisting of TTTTTTTTTATGGCCATCTTGG (SEQ ID NO:422), TTTTTTTTTTAGGCCATCTTGGA (SEQ ID NO:424), TTTTTTTAGATGGCCATCTTG (SEQ ID NO:426), TTTTTTTTGATGGCCATCTTG (SEQ ID NO:428), and TTTTTTTTTTTGGCCATCTTGG (SEQ ID NO:430), (5) a fifth probe comprising a sequence selected from the group consisting of TTTTTTTTTTTGTGATGGCCGG (SEQ ID NO:432), TTTTTTTTTTTTTGATGGCCGGCG (SEQ ID NO:434), TTTTTTTTTTTGTGATGGCCGGCGT (SEQ ID NO:436), TTTTTTTTTTTTTGATGGCCGGCGT (SEQ ID NO:438), and TTTTTTTTTTTTTGATGGCCCGCGTG (SEQ ID NO:440); (6) a sixth probe comprising a sequence selected from the group consisting of TTTTTTTTTTTAACCCCCATCACGT (SEQ ID NO:442), TTTTTTTTGACAACCCCCATCACG (SEQ ID NO:444), TTTTCGTGGACAACCCCCATCA (SEQ ID NO:446), TTTTTTTTTTTTCCCATCACGTGT (SEQ ID NO:448), and TTTTTTTTGGACAACCCCCATCAC (SEQ ID NO:450); and (7) a seventh probe comprising a sequence selected from the group consisting of TTTTTTTTTTTGCCAGCGTGGACGG (SEQ ID NO:452), TTTTTTTTTTTCGTGGACGGTAACC (SEQ ID NO:454), TTTTTTTTTTTGACGGTAACCCCC (SEQ ID NO:456), TTTTTTTTTTCCAGCGTGGACGGT (SEQ ID NO:458), and TTTTTTTGCCAGCGTGGACGGTA (SEQ ID NO:460); wherein each of the seven probes is coupled to a microcarrier with a different identifier. In some embodiments, the kit further comprises a primer pair comprising the sequences CCTCCACCGTGCAGATCATC (SEQ ID NO:15) and TTCCCTGATTACCTTTGCGAT (SEQ ID NO:16); a primer pair comprising the sequences CCACACTGACGTGCCTCT (SEQ ID NO:511) and GCACACGTAGGGGTTGTCCAAGA (SEQ ID NO:512); a primer pair comprising the sequences CCACACTGACGTGCCTCT (SEQ ID NO:513) and GTACACGCTGGCCACGCCG (SEQ ID NO:514); a primer pair comprising the sequences CCACACTGACGTGCCTCT (SEQ ID NO:515) and CAGGCGGCACACGTGAT (SEQ ID NO:516); and/or a primer pair comprising the sequences CCACACTGACGTGCCTCT (SEQ ID NO:517) and AGGCGGCACACGTGCGGGTTAC (SEQ ID NO:518). In some embodiments, the kit further comprises a blocking nucleic acid that hybridizes with a wild-type EGFR DNA locus corresponding with the EGFR DNA mutation and prevents amplification of the wild-type EGFR DNA locus, and wherein the blocking nucleic acid comprises the sequence CATCACGCAGCTCATG(invdT)ₙ, wherein n is 1, 2, or 3 (SEQ ID NO:316); TGCAGCTCATCACGCAGC(invdT)ₙ, wherein n is 1, 2, or 3 (SEQ ID NO:317); TCATCACGCAGCTCAT(invdT)ₙ, wherein n is 1, 2, or 3 (SEQ ID NO:318); TCATCACGCAGC(invdT)ₙ, wherein n is 1, 2, or 3 (SEQ ID NO:319); or CTCATCACGCAGC(invdT)ₙ, wherein n is 1, 2, or 3 (SEQ ID NO:320); with italicized nucleic acids representing locked nucleic acids. In some embodiments, the DNA mutation in the EGFR gene comprises a DNA mutation encoding an L858R mutated EGFR protein. In some embodiments, the probe specific for the DNA mutation in the EGFR gene comprises a sequence selected from the group consisting of ATTTTGGGCGGGCC (SEQ ID NO:151), TTGGGCGGGCCAAA (SEQ ID NO:153), GCGGGCCAAACT (SEQ ID NO:155), GGGCGGGCCAAACT (SEQ ID NO: 157), and TGGGCGGGCCA (SEQ ID NO:159). In some embodiments, the probe further comprises eight nucleotides at the 5′ end, and wherein the eight nucleotides at the 5′ end are adenine or thymine nucleotides. In some embodiments, the probe specific for the DNA mutation in the EGFR gene comprises a sequence selected from the group consisting of TTTTTTTATTTTGGGCGGGCC (SEQ ID NO:152), TTTTTTTTAATTGGGCGGGCCAAA (SEQ ID NO:154), TTTTTTTAAAAAAGCGGGCCAAACT (SEQ ID NO:156), TTTTTTTTAAAAGGGCGGGCCAAACT (SEQ ID NO:158), and TTTTTTTTAAATGGGCGGGCCA (SEQ ID NO:160). In some embodiments, the kit further comprises a primer pair comprising the sequences GGAGGACCGTCGCTTGG (SEQ ID NO:17) and TCTTTCTCTTCCGCACCCAG (SEQ ID NO:18). In some embodiments, the kit further comprises a blocking nucleic acid that hybridizes with a wild-type EGFR DNA locus corresponding with the EGFR DNA mutation and prevents amplification of the wild-type EGFR DNA locus, and wherein the blocking nucleic acid comprises the sequence CCAGCAGTTTGGCCAGCCCT(invdT)ₙ, wherein n is 1, 2, or 3 (SEQ ID NO:321); CCAGCAGTTTGGCCAGCCCT(invdT)ₙ, wherein n is 1, 2, or 3 (SEQ ID NO:322); CCAGCAGTTTGGCCAGCCCT(invdT)ₙ, wherein n is 1, 2, or 3 (SEQ ID NO:323); AGCAGTTTGGCCAGCC(invdT)ₙ, wherein n is 1, 2, or 3 (SEQ ID NO:324); or CCAGCAGTTTGGCCAGCCCT(invdT)ₙ, wherein n is 1, 2, or 3 (SEQ ID NO:325); with italicized nucleic acids representing locked nucleic acids. In some embodiments, the DNA mutation in the AKT1 gene comprises a DNA mutation encoding an E17K mutated AKT1 protein. In some embodiments, the probe specific for the DNA mutation in the AKT1 gene comprises a sequence selected from the group consisting of TGTAGGGAAGTACA (SEQ ID NO:370), TCTGTAGGGAAGTAC (SEQ ID NO:372), GTCTGTAGGGAAGTACAT (SEQ ID NO:374), CCGCACGTCTGTAGGGA (SEQ ID NO:376), and ACGTCTGTAGGGAAGTA (SEQ ID NO:378). In some embodiments, the probe specific for the DNA mutation in the AKT1 gene further comprises seven nucleotides at the 5′ end, and wherein the seven nucleotides at the 5′ end are adenine or thymine nucleotides. In some embodiments, the probe specific for the DNA mutation in the AKT1 gene comprises a sequence selected from the group consisting of TTTTTTTTTTTTTTGTAGGGAAGTACA (SEQ ID NO:371), TTTTTTTTTTTTCTGTAGGGAAGTAC (SEQ ID NO:373), TTTTTTTGTCTGTAGGGAAGTACAT (SEQ ID NO:375), TTTTTTTCCGCACGTCTGTAGGGA (SEQ ID NO:377), and TTTTTTTTACGTCTGTAGGGAAGTA (SEQ ID NO:379). In some embodiments, the kit further comprises a primer pair comprising the sequences GAGGGTCTGACGGGTAGAGTG (SEQ ID NO:380) and TGGCCGCCAGGTCTTGATGTA (SEQ ID NO:381). In some embodiments, the kit further comprises a blocking nucleic acid that hybridizes with a wild-type AKT1 DNA locus corresponding with the AKT1 DNA mutation and prevents amplification of the wild-type AKT1 DNA locus, and wherein the blocking nucleic acid comprises the sequence TGTACTCCCCTACA (invdT)ₙ, wherein n is 1, 2, or 3 (SEQ ID NO:382); GATGTACTCCCCT (invdT)ₙ, wherein n is 1, 2, or 3 (SEQ ID NO:383); ATGTACTCCCCTAC (invdT)ₙ, wherein n is 1, 2, or 3 (SEQ ID NO:384); GTACTCCCCTACA (invdT)ₙ, wherein n is 1, 2, or 3 (SEQ ID NO:385); or GATGTACTCCCCTACA (invdT)ₙ, wherein n is 1, 2, or 3 (SEQ ID NO:386); with italicized nucleic acids representing locked nucleic acids. In some embodiments, the DNA mutation in the MEK1 gene comprises a DNA mutation encoding a K57N mutated MEK1 protein. In some embodiments, the probe specific for the DNA mutation in the MEK1 gene comprises a sequence selected from the group consisting of TTACCCAGAATCAGAA (SEQ ID NO:387), CCAGAATCAGAAGGTG (SEQ ID NO:389), TTCTTACCCAGAATCA (SEQ ID NO:391), CCTTTCTTACCCAGAATC (SEQ ID NO:393), and CAGAATCAGAAGGTGG (SEQ ID NO:395). In some embodiments, the probe specific for the DNA mutation in the MEK1 gene further comprises eight nucleotides at the 5′ end, and wherein the eight nucleotides at the 5′ end are adenine or thymine nucleotides. In some embodiments, the probe specific for the DNA mutation in the MEK1 gene comprises a sequence selected from the group consisting of TTTTTAAATTTACCCAGAATCAGAA (SEQ ID NO:388), TTTTTAAATCCAGAATCAGAAGGTG (SEQ ID NO:390), TTTTTAAATTTCTTACCCAGAATCA (SEQ ID NO:392), TTTTTAAATCCTTTCTTACCCAGAATC (SEQ ID NO:394), and TTTTTAAATCAGAATCAGAAGGTGG (SEQ ID NO:396). In some embodiments, the kit further comprises a primer pair comprising the sequences CTTGATGAGCAGCAGCGAAA (SEQ ID NO:397) and CCTTCAGTTCTCCCACCTTCTG (SEQ ID NO:398). In some embodiments, the kit further comprises a blocking nucleic acid that hybridizes with a wild-type MEK1 DNA locus corresponding with the MEK1 DNA mutation and prevents amplification of the wild-type MEK1 DNA locus, and wherein the blocking nucleic acid comprises the sequence TCTGCTTCTGGGTAAG (invdT)ₙ, wherein n is 1, 2, or 3 (SEQ ID NO:399); TTCTGCTTCTGGGTAAGA (invdT)ₙ, wherein n is 1, 2, or 3 (SEQ ID NO:400); CACCTTCTGCTTCTGGG (invdT)ₙ, wherein n is 1, 2, or 3 (SEQ ID NO:401); TCTGCTTCTGGGTA (invdT)ₙ, wherein n is 1, 2, or 3 (SEQ ID NO:402); or CACCTTCTGCTTCTGGGTAAGA (invdT)ₙ, wherein n is 1, 2, or 3 (SEQ ID NO:403); with italicized nucleic acids representing locked nucleic acids. In some embodiments, the DNA mutation in the HER2 gene comprises a DNA mutation encoding an A775_G776insYVMA mutated HER2 protein. In some embodiments, the probe specific for the DNA mutation in the HER2 gene comprises a sequence selected from the group consisting of ATACGTGATGTCTTAC (SEQ ID NO:404), ACGTGATGGCTTACGT (SEQ ID NO:406), AAGCATACGTGATGGCT (SEQ ID NO:408), GCATACGTGATGGCTT (SEQ ID NO:410), and GCATACGTGATGGCTTA (SEQ ID NO:412). In some embodiments, the probe specific for the DNA mutation in the HER2 gene further comprises five nucleotides at the 5′ end, and wherein the five nucleotides at the 5′ end are adenine or thymine nucleotides. In some embodiments, the probe specific for the DNA mutation in the HER2 gene comprises a sequence selected from the group consisting of TTTTTTTTTATACGTGATGTCTTAC (SEQ ID NO:405), TTTTTTTTTTTACGTGATGGCTTACGT (SEQ ID NO:407), TTTTTAAGCATACGTGATGGCT (SEQ ID NO:409), TTTTTTTGCATACGTGATGGCTT (SEQ ID NO:411), and TTTTTTTGCATACGTGATGGCTTA (SEQ ID NO:413). In some embodiments, the kit further comprises a primer pair comprising the sequences ATGGCTGTGGTTTGTGATGGT (SEQ ID NO:414) and ACACCAGCCATCACGTAAGACA (SEQ ID NO:415).

In another aspect, provided herein is a kit comprising at least five microcarriers, wherein each of said at least five microcarriers comprises: (i) a probe coupled to the microcarrier, wherein the probe is specific for an RNA mutation in the ALK, ROS, RET, NTRK1, or cMET gene; and (ii) an identifier corresponding to the probe coupled thereto; wherein the kit comprises at least one microcarrier comprising a probe specific for an RNA mutation in the ALK gene, at least one microcarrier comprising a probe specific for an RNA mutation in the ROS gene, at least one microcarrier comprising a probe specific for an RNA mutation in the RET gene, at least one microcarrier comprising a probe specific for an RNA mutation in the NTRK1 gene, and at least one microcarrier comprising a probe specific for an RNA mutation in the cMET gene; and wherein the ALK, ROS, RET, NTRK1, and cMET genes are human genes. In some embodiments, each of the mutations in the ALK, ROS, RET, and NTRK1 genes comprises a fusion gene or gene rearrangement.

In some embodiments, the mutation in the ALK gene comprises one or more of EML E13:ALK E20, EML E20:ALK E20, and EML E6:ALK E20 EML4-ALK fusion genes. In some embodiments, the mutation in the ALK gene comprises EML E13:ALK E20, EML E20:ALK E20, and EML E6:ALK E20 EML4-ALK fusion genes. In some embodiments, the probe specific for the mutation in the ALK gene comprises: (1) a first probe comprising a sequence selected from the group consisting of AAAGGACCTAAAGTGT (SEQ ID NO:161), CCTAAAGTGTACCGC (SEQ ID NO:163), GGGAAAGGACCTAAAG (SEQ ID NO:165), AGTGTACCGCCGGAA (SEQ ID NO:167), and TACCGCCGGAAGCACC (SEQ ID NO:169); (2) a second probe comprising a sequence selected from the group consisting of GACTATGAAATATTGTAC (SEQ ID NO:171), GAAATATTGTACTTGTAC (SEQ ID NO:173), TATTGTACTTGTACCGCC (SEQ ID NO:175), TGTACCGCCGGAAGCAC (SEQ ID NO:177), and CCGCCGGAAGCACCAGGA (SEQ ID NO:179); (3) a third probe comprising a sequence selected from the group consisting of TGTCATCATCAACCAA (SEQ ID NO:181), ATGTCATCATCAACC (SEQ ID NO:183), GTGTACCGCCGGAAGC (SEQ ID NO:185), TCAACCAAGTGTACCG (SEQ ID NO:187), and TACCGCCGGAAGCACCA (SEQ ID NO:189); and (4) a fourth probe comprising a sequence selected from the group consisting of CGAAAAAAACAGCCAA (SEQ ID NO:191), TCGCGAAAAAAACAGC (SEQ ID NO:193), GTGTACCGCCGGAAGC (SEQ ID NO:195), TACCGCCGGAAGCACC (SEQ ID NO:197), and ACAGCCAAGTGTACCG (SEQ ID NO:199); wherein each of the four probes is coupled to a microcarrier with a different identifier. In some embodiments, each of the four probes further comprises eight nucleotides at the 5′ end, and wherein the eight nucleotides at the 5′ end are adenine or thymine nucleotides. In some embodiments, the probe specific for the mutation in the ALK gene comprises: (1) a first probe comprising a sequence selected from the group consisting of TTTTTTTTTTAAAGGACCTAAAGTGT (SEQ ID NO:162), TTTTTTTTTTCCTAAAGTGTACCGC (SEQ ID NO:164), TTTTTTTTTTGGGAAAGGACCTAAAG (SEQ ID NO:166), TTTTTTTTTTAGTGTACCGCCGGAA (SEQ ID NO:168), and TTTTTTTTTTTACCGCCGGAAGCACC (SEQ ID NO: 170); (2) a second probe comprising a sequence selected from the group consisting of TTTTTTTTTTTTGACTATGAAATATTGTAC (SEQ ID NO:172), TTTTTTTTTTTTGAAATATTGTACTTGTAC (SEQ ID NO:174), TTTTTTTTTTTTTATTGTACTTGTACCGCC (SEQ ID NO:176), TTTTTTTTTTTTTTGTACCGCCGGAAGCAC (SEQ ID NO:178), and TTTTTTTTTTTTCCGCCGGAAGCACCAGGA (SEQ ID NO:180); (3) a third probe comprising a sequence selected from the group consisting of TTTTTTTTTTTTTTTGTCATCATCAACCAA (SEQ ID NO:182), TTTTTTTTTTTTTTATGTCATCATCAACC (SEQ ID NO:184), TTTTTTTTTTTTTTGTGTACCGCCGGAAGC (SEQ ID NO:186), TTTTTTTTTTTTTTTCAACCAAGTGTACCG (SEQ ID NO:188), and TTTTTTTTTTTTTTTACCGCCGGAAGCACCA (SEQ ID NO:190); and (4) a fourth probe comprising a sequence selected from the group consisting of TTTTTTTTTTTTTCGAAAAAAACAGCCAA (SEQ ID NO:192), TTTTTTTTTTTTTTCGCGAAAAAAACAGC (SEQ ID NO:194), TTTTTTTTTTTTTGTGTACCGCCGGAAGC (SEQ ID NO:196), TITTTTTTTTTTTTACCGCCGGAAGCACC (SEQ ID NO:198), and TTTTTTTTTTTTTTACAGCCAAGTGTACCG (SEQ ID NO:200); wherein each of the four probes is coupled to a microcarrier with a different identifier. In some embodiments, the kit further comprises a first primer that is suitable for generating cDNA specific for the mutation in the ALK gene, wherein the first primer comprises the sequence AGTTGGGGTTGTAGTCGGTCAT (SEQ ID NO:363) or GAAGCCTCCCTGGATCTCC (SEQ ID NO:364); and a second primer specific for the mutation in the ALK gene that comprises a sequence selected from the group consisting of TATGGAGCAAAACTACTGTAGAGCC (SEQ ID NO:357), CCAGCTACATCACACACCTTGACT (SEQ ID NO:358), and TAATACCAAAAGTTACCAAAACTGCA (SEQ ID NO:359). In some embodiments, the mutation in the ROS gene comprises an ROS fusion gene selected from the group consisting of CD74-ROS, and SLC34A2-ROS. In some embodiments, the mutation in the ROS gene comprises CD74 E6:ROS E32, CD74 E6:ROS E34, SLC34A2 E4:ROS E32, and SLC34A2 E4:ROS E34 fusion genes. In some embodiments, the probe specific for the mutation in the ROS gene comprises: (1) a first probe comprising a sequence selected from the group consisting of ACTGACGCTCCACCGAAA (SEQ ID NO:201), CCACTGACGCTCCACCGA (SEQ ID NO:203), GCTGGAGTCCCAAATAAAC (SEQ ID NO:205), GGAGTCCCAAATAAACCAG (SEQ ID NO:207), and CACCGAAAGCTGGAGTCCC (SEQ ID NO:209); (2) a second probe comprising a sequence selected from the group consisting of CCGAAAGATGATTTT (SEQ ID NO:211), GACGCTCCACCGAAA (SEQ ID NO:213), ACTGACGCTCCACCGA (SEQ ID NO:215), GATGATTTTTGGATA (SEQ ID NO:217), and TGATTTTTGGATACCA (SEQ ID NO:219); (3) a third probe comprising a sequence selected from the group consisting of AGCGCCTTCCAGCTGGTTGGA (SEQ ID NO:221), CTGGTTGGAGCTGGAGTCCC (SEQ ID NO:223), AGTAGCGCCTTCCAGCTGGTTG (SEQ ID NO:225), GCTGGAGTCCCAAATAAACCA (SEQ ID NO:227), and GGAGTCCCAAATAAACCAGG (SEQ ID NO:229); and (4) a fourth probe comprising a sequence selected from the group consisting of GCGCCTTCCAGCTGGTTG (SEQ ID NO:231), GTAGCGCCTTCCAGCTGGT (SEQ ID NO:233), TGGTTGGAGATGATTTTT (SEQ ID NO:235), GATGATTTTTGGATACCAG (SEQ ID NO:237), and TGATTTTTGGATACCA (SEQ ID NO:239); wherein each of the four probes is coupled to a microcarrier with a different identifier. In some embodiments, each of the four probes further comprises eight nucleotides at the 5′ end, and wherein the eight nucleotides at the 5′ end are adenine or thymine nucleotides. In some embodiments, the probe specific for the mutation in the ROS gene comprises: (1) a first probe comprising a sequence selected from the group consisting of TTTTTTTTTTTACTGACGCTCCACCGAAA (SEQ ID NO:202), TTTTTTTTTTTCCACTGACGCTCCACCGA (SEQ ID NO:204), TTTTTTTTTTTGCTGGAGTCCCAAATAAAC (SEQ ID NO:206), TTTTTTTTTTTGGAGTCCCAAATAAACCAG (SEQ ID NO:208), and TTTTTTTTTTTCACCGAAAGCTGGAGTCCC (SEQ ID NO:210); (2) a second probe comprising a sequence selected from the group consisting of TTTTTTTTTTTTCCGAAAGATGATTTT (SEQ ID NO:212), TTTTTTTTTTTTGACGCTCCACCGAAA (SEQ ID NO:214), TTTTTTTTTTTTACTGACGCTCCACCGA (SEQ ID NO:216), TTTTTTTTTTTTGATGATTTTTGGATA (SEQ ID NO:218), and TTTTTTTTTTTTTGATTTTTGGATACCA (SEQ ID NO:220); (3) a third probe comprising a sequence selected from the group consisting of TTTTTTTTTTTTAGCGCCTTCCAGCTGGTTGGA (SEQ ID NO:222), TTTTTTTTTTTTCTGGTTGGAGCTGGAGTCCC (SEQ ID NO:224), TTTTTTTTTTTTAGTAGCGCCTTCCAGCTGGTTG (SEQ ID NO:226), TTTTTTTTTTTTGCTGGAGTCCCAAATAAACCA (SEQ ID NO:228), and TTTTTTTTTTTTGGAGTCCCAAATAAACCAGG (SEQ ID NO:230); and (4) a fourth probe comprising a sequence selected from the group consisting of TTTTTTTTTTGCGCCTTCCAGCTGGTTG (SEQ ID NO:232), TTTTTTTTTTGTAGCGCCTTCCAGCTGGT (SEQ ID NO:234), TTTTTTTTTTTGGTTGGAGATGATTTTT (SEQ ID NO:236), TTTTTTTTTTGATGATTTTTGGATACCAG (SEQ ID NO:238), and TTTTTTTTTTTGATTTTTGGATACCA (SEQ ID NO:240); wherein each of the four probes is coupled to a microcarrier with a different identifier. In some embodiments, the kit further comprises a first primer that is suitable for generating cDNA specific for the mutation in the ROS gene, wherein the first primer comprises the sequence AATTCAATACATACTATCAGCTTTCTCCCACTGTATTGAA (SEQ ID NO:21) or AATATTTCTGGTACGAGTGGGATTGTAACAACCAGAAATA (SEQ ID NO:22); and a second primer specific for the mutation in the ROS gene that comprises the sequence GGAGTGCCATCGCTGTTTGAAATGAGCAGGCACT (SEQ ID NO:19) or TACAGCCCTGGATATTCTTAGTAGCGC (SEQ ID NO:20). In some embodiments, the mutation in the RET gene comprises a RET fusion gene selected from the group consisting of KIF5B-RET. In some embodiments, the mutation in the RET gene comprises KIF5B E15:RET E11, KIF5B E15:RET E12, KIF5B E16:RET E12, KIF5B E22:RET E12, and KIF5B E23:RET E12 fusion genes. In some embodiments, the probe specific for the mutation in the RET gene comprises: (1) a first probe comprising a sequence selected from the group consisting of GTGGGAAATAATGATGTAAA (SEQ ID NO:241), CTGTGGGAAATAATGATGTA (SEQ ID NO:243), GATCCACTGTGCGACGAGCT (SEQ ID NO:245), TGATGTAAAGATCCACTGTG (SEQ ID NO:247), and TCCACTGTGCGACGAGCTGT (SEQ ID NO:249); (2) a second probe comprising a sequence selected from the group consisting of TGGGAAATAATGATGTAAA (SEQ ID NO:251), CTGTGGGAAATAATGATGTA (SEQ ID NO:253), GGAGGATCCAAAGTGGGAAT (SEQ ID NO:255), GGATCCAAAGTGGGAATT (SEQ ID NO:257), and ATGATGTAAAGGAGGATCC (SEQ ID NO:259); (3) a third probe comprising a sequence selected from the group consisting of CTTCGTATCTCTCAAGAGGAT (SEQ ID NO:481), GTATCTCTCAAGAGGATCCAA (SEQ ID NO:483), TTCGTATCTCTCAAGAG (SEQ ID NO:485), TCAAGAGGATCCAAA (SEQ ID NO:487), and TCTCTCAAGAGG (SEQ ID NO:489); (4) a fourth probe comprising a sequence selected from the group consisting of GTTAAAAAGGAGGATCCAA (SEQ ID NO:491), ACAAGAGTTAAAAAGGAGGA (SEQ ID NO:493), AAGAGTTAAAAAGGAGGATC (SEQ ID NO:495), AAAAGGAGGATCCAAAG (SEQ ID NO:497), and AAGGAGGATCCAAAGTG (SEQ ID NO:499); and (5) a fifth probe comprising a sequence selected from the group consisting of AAACAGGAGGATCCAAA (SEQ ID NO:501), AAGTGCACAAACAGGAGG (SEQ ID NO:503), GTGCACAAACAGGAGGATC (SEQ ID NO:505), CACAAACAGGAGGAT (SEQ ID NO:507), and AACAGGAGGATCCAAA (SEQ ID NO:509); wherein each of the five probes is coupled to a incrocarrier with a different identifier. In some embodiments, each of the four probes further comprises eight nucleotides at the 5′ end, and wherein the eight nucleotides at the 5′ end are adenine or thymine nucleotides. In some embodiments, the probe specific for the mutation in the RET gene comprises: (1) a first probe comprising a sequence selected from the group consisting of TTTTTTTTTTGTGGGAAATAATGATGTAAA (SEQ ID NO:242), TTTTTTTTTTCTGTGGGAAATAATGATGTA (SEQ ID NO:244), TTTTTTTTTTGATCCACTGTGCGACGAGCT (SEQ ID NO:246), TTTTTTTTTTTGATGTAAAGATCCACTGTG (SEQ ID NO:248), and TTTTTTTTTTTCCACTGTGCGACGAGCTGT (SEQ ID NO:250); (2) a second probe comprising a sequence selected from the group consisting of TTTTTTTTTTGGGAAATAATGATGTAAA (SEQ ID NO:252), TTTTTTTTTCTGTGGGAAATAATGATGTA (SEQ ID NO:254), TTTTTTTTTGGAGGATCCAAAGTGGGAAT (SEQ ID NO:256), TTTTTTTTTGGATCCAAAGTGGGAATT (SEQ ID NO:258), and TTTTTTTTTATGATGTAAAGGAGGATCC (SEQ ID NO:260); (3) a third probe comprising a sequence selected from the group consisting of TTTTTTTTTCTTCGTATCTCTCAAGAGGAT (SEQ ID NO:482), TTTTTTTTTGTATCTCTCAAGAGGATCCAA (SEQ ID NO:484), TTTTTTTTTTTCGTATCTCTCAAGAG (SEQ ID NO:486), TTTTTTTTTTCAAGAGGATCCAAA (SEQ ID NO:488), and TTTTTTTTTTCTCTCAAGAGG (SEQ ID NO:490); (4) a fourth probe comprising a sequence selected from the group consisting of TTTTTTTTTGTTAAAAAGGAGGATCCAA (SEQ ID NO:492), TTTTTTTTACAAGAGTTAAAAAGGAGGA (SEQ ID NO:494), TTATTATTAAGAGTTAAAAAGGAGGATC (SEQ ID NO:811), TTTTTTTTAAAAGGAGGATCCAAAG (SEQ ID NO:498), and TTTTTTTTAAGGAGGATCCAAAGTG (SEQ ID NO:500); and (5) a fifth probe comprising a sequence selected from the group consisting of TTTTTTTTAAACAGGAGGATCCAAA (SEQ ID NO:502), TTTTTATTAAGTGCACAAACAGGAGG (SEQ ID NO:504), TATTATTATGTGCACAAACAGGAGGATC (SEQ ID NO:506), TATTTTTTCACAAACAGGAGGAT (SEQ ID NO:508), and TTTTATTTAACAGGAGGATCCAAA (SEQ ID NO:510); wherein each of the five probes is coupled to a microcarrier with a different identifier. In some embodiments, the kit further comprises a first primer that is suitable for generating cDNA specific for the mutation in the RET gene, wherein the first primer comprises the sequence GTGATCGCACAGTAGGACAGCGGCTGCGATC (SEQ ID NO:26) or CTCTAGGAGATATCATTCCAAATTCGCCTTCTCCTAG (SEQ ID NO:27); and a second primer specific for the mutation in the RET gene that comprises a sequence selected from the group consisting of TTTCTGGTGCTATGAGGAAATGACCAACCACCAGA (SEQ ID NO:23), AAGGAGTTAGCAGCATGTCAGC (SEQ ID NO:519), AACTTCAGACTTTACACAACCTGC (SEQ ID NO:520), and ATTGATTCTGATGACACCGGA (SEQ ID NO:521). In some embodiments, the mutation in the NTRK1 gene comprises a CD74-NTRK1 fusion gene. In some embodiments, the mutation in the NTRK1 gene comprises a CD74 E8:NTRK1 E12 fusion gene. In some embodiments, the probe specific for the mutation in the NTRK1 gene comprises a sequence selected from the group consisting of CAGGATCTGGGCCCAGACA (SEQ ID NO:261), GATCTGGGCCCAGACACTA (SEQ ID NO:263), CCAGACACTAACAGCACAT (SEQ ID NO:265), GGGCCCAGACACTAACAGC (SEQ ID NO:267), and CTAACAGCACATCTGGAGA (SEQ ID NO:269). In some embodiments, the probe specific for the mutation in the NTRK1 gene further comprises eight nucleotides at the 5′ end, and wherein the eight nucleotides at the 5′ end are adenine or thymine nucleotides. In some embodiments, the probe specific for the mutation in the NTRK1 gene comprises a sequence selected from the group consisting of TTTTTTTTTTACAGGATCTGGGCCCAGACA (SEQ ID NO:262), TTTTTTTTTTAGATCTGGGCCCAGACACTA (SEQ ID NO:264), TTTTTTTTTTACCAGACACTAACAGCACAT (SEQ ID NO:266), TTTTTTTTTTAGGGCCCAGACACTAACAGC (SEQ ID NO:268), and TTTTTTTTTTACTAACAGCACATCTGGAGA (SEQ ID NO:270). In some embodiments, the kit further comprises a first primer that is suitable for generating cDNA specific for the mutation in the NTRK1 gene, wherein the first primer comprises the sequence GGACGAAAATCCAGACCCCAAAAGGTGTTTCGT (SEQ ID NO:32); and a second primer specific for the mutation in the NTRK1 gene that comprises the sequence AGAAGACGTGACAGGAACTGGAGGACCCGTCTT (SEQ ID NO:30). In some embodiments, the mutation in the cMET gene results in exon skipping. In some embodiments, the mutation in the cMET gene results in skipping of exon 14. In some embodiments, the probe specific for the mutation in the cMET gene comprises a sequence selected from the group consisting of AGAAAGCAAATTAAAGAT (SEQ ID NO:271), AGCAAATTAAAGATCAG (SEQ ID NO:273), AAATTAAAGATCAGTTTC (SEQ ID NO:275), AGATCAGTTTCCTAATTC (SEQ ID NO:277), and AAGATCAGTTTCCTAATT (SEQ ID NO:279). In some embodiments, the probe specific for one or more mutations in the cMET gene further comprises eight nucleotides at the 5′ end, and wherein the eight nucleotides at the 5′ end are adenine or thymine nucleotides. In some embodiments, the probe specific for the mutation in the cMET gene comprises a sequence selected from the group consisting of TTTTTTTTTTAGAAAGCAAATTAAAGAT (SEQ ID NO:272), TTTTTTTTTTAGCAAATTAAAGATCAG (SEQ ID NO:274), TTTTTTTTTTAAATTAAAGATCAGTTTC (SEQ ID NO:276), TTTTTTTTTTAGATCAGTTTCCTAATTC (SEQ ID NO:278), and TTTTTTTTITAAGATCAGTTTCCTAATT (SEQ ID NO:280). In some embodiments, the kit further comprises a first primer that is suitable for generating cDNA specific for the mutation in the cMET gene, wherein the first primer comprises the sequence GACAGTATTTTGCAGTAATGGACTGGATATATCAGA (SEQ ID NO:29); and a second primer specific for the mutation in the cMET gene that comprises the sequence GAATTTCACAGGATTGATTGCTGGTGTTGTCTC (SEQ ID NO:28).

In some embodiments according to any of the embodiments described herein, the identifiers of the microcarriers comprise digital barcodes. In some embodiments, each of the microcarriers comprises: (i) a first photopolymer layer; (ii) a second photopolymer layer; and (iii) an intermediate layer between the first layer and the second layer, the intermediate layer having an encoded pattern representing the identifier defined thereon, wherein the intermediate layer is partially substantially transmissive and partially substantially opaque to light, representing a code corresponding to the microcarrier, wherein the outermost surface of the microcarrier comprises a photoresist photopolymer, and said photoresist photopolymer is functionalized with the probe specific for the DNA mutation, and wherein said microcarrier has about the same density as water. In some embodiments, the identifiers of the microcarriers comprise analog codes. In some embodiments, each of the microcarriers comprises: (i) a substantially transparent polymer layer having a first surface and a second surface, the first and the second surfaces being parallel to each other; (ii) a substantially non-transparent layer that constitutes a two-dimensional shape, wherein the substantially non-transparent layer is affixed to the first surface of the substantially transparent polymer layer and encloses a center portion of the substantially transparent polymer layer, wherein the two-dimensional shape of the substantially non-transparent layer represents an analog code, and wherein the analog code corresponds to the identifier; and (iii) the probe specific for the mutation, wherein the probe is coupled to at least one of the first surface and the second surface of the substantially transparent polymer layer in at least the center portion of the substantially transparent polymer layer. In some embodiments, each of the microcarriers further comprises an orientation indicator for orienting the analog code of the substantially non-transparent polymer layer. In some embodiments, the polymer of the substantially transparent polymer layer comprises an epoxy-based polymer. In some embodiments, the epoxy-based polymer is SU-8.

In another aspect, provided herein is a kit comprising: (a) a plurality of probes, wherein each probe of the plurality is coupled to a microcarrier that has a unique identifier corresponding to the probe coupled thereto, the plurality of probes comprising a first probe comprising the sequence TTTTTTTTTTTTAATAGTTGGAGCT (SEQ ID NO:39); a second probe comprising the sequence TTTTTTTTTTTAGGCGTAGGCAAGA (SEQ ID NO:57); a third probe comprising the sequence TTTTTTTTTTTAAGGAGCTTGTGGC (SEQ ID NO:63); a fourth probe comprising the sequence TTTTTTTCCTGCTCAGTGATTTTA (SEQ ID NO: 94); a fifth probe comprising the sequence TTTTTTTTTTTATCTCCTGCTTAG (SEQ ID NO:102); a sixth probe comprising the sequence TTTTTTTTTTTTAATGATGCACGTCA (SEQ ID NO:114); a seventh probe comprising the sequence TTTTTTAATTCTACAGAGAAATCTCGA (SEQ ID NO:82); an eighth probe comprising the sequence TTTTTTTTTTTTTAGTGCTGGCCT (SEQ ID NO:124); a ninth probe comprising the sequence TTTTTTTTTTTACAAAACATCTCCG (SEQ ID NO:131); a tenth probe comprising the sequence TTTTTTTTAATCAAGACATCTC (SEQ ID NO:141); an eleventh probe comprising the sequence TTTTTTTTCATGAGATGCATGATGA (SEQ ID NO:356); a twelfth probe comprising the sequence TTTTTTTTTTTACAGGAGGCTGCCGA (SEQ ID NO:464); a thirteenth probe comprising the sequence TTTTTTTTTTTACAGGAGGGAGCCG (SEQ ID NO:478); a fourteenth probe comprising the sequence TTTTTTTAGATGGCCATCTTG (SEQ ID NO:426); a fifteenth probe comprising the sequence TTTTTTTTTTTGTGATGGCCGG (SEQ ID NO:432); a sixteenth probe comprising the sequence TTTTTTTTGGACAACCCCCATCAC (SEQ ID NO:450); a seventeenth probe comprising the sequence TTTTTTTGCCAGCGTGGACGGTA (SEQ ID NO:460); an eighteenth probe comprising the sequence TTTTTTTTAAATGGGCGGGCCA (SEQ ID NO:160); a nineteenth probe comprising the sequence TTTTTTTTTTTTTTGTAGGGAAGTACA (SEQ ID NO:371); a twentieth probe comprising the sequence TTTTTAAATCAGAATCAGAAGGTGG (SEQ ID NO:396); a twentieth probe comprising the sequence TTTTTAAATCAGAATCAGAAGGTGG (SEQ ID NO:396); a twenty-first probe comprising the sequence TTTTTTTTTTTACGTGATGGCTTACGT (SEQ ID NO:407); a twenty-second probe comprising the sequence TTTTTTTTTTAGTGTACCGCCGGAA (SEQ ID NO:168); a twenty-third probe comprising the sequence TTTTTTTTTTTTGACTATGAAATATTGTAC (SEQ ID NO:172); a twenty-fourth probe comprising the sequence TTTTTTTTTTTTTTTACCGCCGGAAGCACCA (SEQ ID NO:190); a twenty-fifth probe comprising the sequence TTTTTTTTTTTTTGTGTACCGCCGGAAGC (SEQ ID NO:196); a twenty-sixth probe comprising the sequence TTTTTTTTTTTGCTGGAGTCCCAAATAAAC (SEQ ID NO:206); a twenty-seventh probe comprising the sequence TTTTTTTTTTTTGACGCTCCACCGAAA (SEQ ID NO:214); a twenty-eighth probe comprising the sequence TTTTTTTTTTTTGGAGTCCCAAATAAACCAGG (SEQ ID NO:230); a twenty-ninth probe comprising the sequence TTTTTTTTTTGATGATTTTTGGATACCAG (SEQ ID NO:238); a thirtieth probe comprising the sequence TTTTTTTTTTGTGGGAAATAATGATGTAAA (SEQ ID NO:242); a thirty-first probe comprising the sequence TTTTTTTTTCTGTGGGAAATAATGATGTA (SEQ ID NO:254); a thirty-second probe comprising the sequence TTTTTTTTTTCTCTCAAGAGG (SEQ ID NO:490); a thirty-third probe comprising the sequence TTTTTTTTAAGGAGGATCCAAAGTG (SEQ ID NO:500); a thirty-fourth probe comprising the sequence TTTTTTTTAAACAGGAGGATCCAAA (SEQ ID NO:502); a thirty-fifth probe comprising the sequence TTTTTTTTTTACCAGACACTAACAGCACAT (SEQ ID NO:266); a thirty-sixth probe comprising the sequence TTTTTTTTTTAGAAAGCAAATTAAAGAT (SEQ ID NO:272); (b) a plurality of primer pairs, the plurality of primer pairs comprising a first primer pair comprising the sequences GTACTGGTGGAGTATTTGATAGTG (SEQ ID NO:1) and CGTCAAGGCACTCTTGCCTAC (SEQ ID NO:2); a second primer pair comprising the sequences CAATTTCTACAAGAGATCCTCTCTCT (SEQ ID NO:5) and CTCCATTTTAGCACTTACCTGTGAC (SEQ ID NO:6); a third primer pair comprising the sequences ACCCTAGCCTTAGATAAAACTGAGC (SEQ ID NO:7) and TTTGTTGTCCAGCCACCATGA (SEQ ID NO:8); a fourth primer pair comprising the sequences ATAGCCTCAATTCTTACCATCCACAAAATG (SEQ ID NO:9) and CAGATATATTTCTTCATGAAGACCTCACAGTAA (SEQ ID NO: 10); a fifth primer pair comprising the sequences CTTGTGGAGCCTCTTACACCC (SEQ ID NO:11) and TGCCGAACGCACCGGA (SEQ ID NO:12); a sixth primer pair comprising the sequences GCCAGTTAACGTCTTCCTTCTC (SEQ ID NO:13) and ATCGAGGATTTCCTTGTTGGCTT (SEQ ID NO:14); a seventh primer pair comprising the sequences CCTCCACCGTGCAGATCATC (SEQ ID NO:15) and TTCCCTGATTACCTTTGCGAT (SEQ ID NO:16); an eighth primer pair comprising the sequences CCACACTGACGTGCCTCT (SEQ ID NO:511) and GCACACGTAGGGGTTGTCCAAGA (SEQ ID NO:512); a ninth primer pair comprising the sequences CCACACTGACGTGCCTCT (SEQ ID NO:513) and GTACACGCTGGCCACGCCG (SEQ ID NO:514); a tenth primer pair comprising the sequences CCACACTGACGTGCCTCT (SEQ ID NO:515) and CAGGCGGCACACGTGAT (SEQ ID NO:516); an eleventh primer pair comprising the sequences CCACACTGACGTGCCTCT (SEQ ID NO:517) and AGGCGGCACACGTGCGGGTTAC (SEQ ID NO:518); a twelfth primer pair comprising the sequences GGAGGACCGTCGCTTGG (SEQ ID NO:17) and TCTTTCTCTTCCGCACCCAG (SEQ ID NO:18); a thirteenth primer pair comprising the sequences GAGGGTCTGACGGGTAGAGTG (SEQ ID NO:380) and TGGCCGCCAGGTCTTGATGTA (SEQ ID NO:381); a fourteenth primer pair comprising the sequences CTTGATGAGCAGCAGCGAAA (SEQ ID NO:397) and CCTTCAGTTCTCCCACCTTCTG (SEQ ID NO:398); a fifteenth primer pair comprising the sequences ATGGCTGTGGTTTGTGATGGT (SEQ ID NO:414) and ACACCAGCCATCACGTAAGACA (SEQ ID NO:415); a sixteenth primer pair comprising the sequences AGTTGGGGTTGTAGTCGGTCAT (SEQ ID NO:363) and TATGGAGCAAAACTACTGTAGAGCC (SEQ ID NO:357); a seventeenth primer pair comprising the sequences AGTTGGGGTTGTAGTCGGTCAT (SEQ ID NO:363) and CCAGCTACATCACACACCTTGACT (SEQ ID NO:358); an eighteenth primer pair comprising the sequences AGTTGGGGTTGTAGTCGGTCAT (SEQ ID NO:363) and TAATACCAAAAGTTACCAAAACTGCA (SEQ ID NO:359); a nineteenth primer pair comprising the sequences GGAGTGCCATCGCTGTTTGAAATGAGCAGGCACT (SEQ ID NO:19); a twentieth primer pair comprising the sequences GGAGTGCCATCGCTGTTTGAAATGAGCAGGCACT (SEQ ID NO:19); a twenty-first primer pair comprising the sequences TACAGCCCTGGATATTCTTAGTAGCGC (SEQ ID NO:20); a twenty-second primer pair comprising the sequences TACAGCCCTGGATATTCTTAGTAGCGC (SEQ ID NO:20); a twenty-third primer pair comprising the sequences GTGATCGCACAGTAGGACAGCGGCTGCGATC (SEQ ID NO:26) and TTTCTGGTGCTATGAGGAAATGACCAACCACCAGA (SEQ ID NO:23); a twenty-fourth primer pair comprising the sequences CTCTAGGAGATATCATTCCAAATTCGCCTTCTCCTAG (SEQ ID NO:27) and TTTCTGGTGCTATGAGGAAATGACCAACCACCAGA (SEQ ID NO:23); a twenty-fifth primer pair comprising the sequences CTCTAGGAGATATCATTCCAAATTCGCCTTCTCCTAG (SEQ ID NO:27) and AAGGAGTTAGCAGCATGTCAGC (SEQ ID NO:519); a twenty-sixth primer pair comprising the sequences CTCTAGGAGATATCATTCCAAATTCGCCTTCTCCTAG (SEQ ID NO:27) and AACTTCAGACTTTACACAACCTGC (SEQ ID NO:520); a twenty-seventh primer pair comprising the sequences CTCTAGGAGATATCATTCCAAATTCGCCTTCTCCTAG (SEQ ID NO:27) and ATTGATTCTGATGACACCGGA (SEQ ID NO:521); a twenty-eighth primer pair comprising the sequences GGACGAAAATCCAGACCCCAAAAGGTGTTTCGT (SEQ ID NO:32) and AGAAGACGTGACAGGAACTGGAGGACCCGTCTT (SEQ ID NO:30); a twenty-ninth primer pair comprising the sequences GACAGTATTTTGCAGTAATGGACTGGATATATCAGA (SEQ ID NO:29) and GAATTTCACAGGATTGATTGCTGGTGTTGTCTC (SEQ ID NO:28); and (c) a plurality of blocking nucleic acids, the plurality of blocking nucleic acids comprising a first blocking nucleic acid comprising the sequence TTGGAGCTGGTGGCGT(invdT)ₙ, wherein n is 1, 2, or 3 (SEQ ID NO:285); a second blocking nucleic acid comprising the sequence CTGAAATCACTGAGCAGG(invdT)ₙ, wherein n is 1, 2, or 3 (SEQ ID NO:291); a third blocking nucleic acid comprising the sequence CCACCATGATGTGCATCA(invdT)ₙ, wherein n is 1, 2, or 3 (SEQ ID NO:299); a fourth blocking nucleic acid comprising the sequence GAGATTTCACTGTAGC(invdT)ₙ, wherein n is 1, 2, or 3 (SEQ ID NO:301); a fifth blocking nucleic acid comprising the sequence CGCACCGGAGCCCAGCACTTA (invdT)ₙ, wherein n is 1, 2, or 3 (SEQ ID NO:310); a sixth blocking nucleic acid comprising the sequence CGGAGATGTTGCTTCTCT(invdT)ₙ, wherein n is 1, 2, or 3 (SEQ ID NO:312); a seventh blocking nucleic acid comprising the sequence TGCAGCTCATCACGCAGC(invdT)ₙ, wherein n is 1, 2, or 3 (SEQ ID NO:317); an eighth blocking nucleic acid comprising the sequence CCAGCAGTTTGGCCAGCCCT(invdT)ₙ, wherein n is 1, 2, or 3 (SEQ ID NO:322); a ninth blocking nucleic acid comprising the sequence GATGTACTCCCCT (invdT)ₙ, wherein n is 1, 2, or 3 (SEQ ID NO:383); a tenth blocking nucleic acid comprising the sequence CACCTTCTGCTTCTGGGTAAGA (invdT)ₙ, wherein n is 1, 2, or 3 (SEQ ID NO:403).

In another aspect, provided herein is a kit comprising: (a) a plurality of probes, wherein each probe of the plurality is coupled to a microcarrier that has a unique identifier corresponding to the probe coupled thereto, the plurality of probes comprising a first probe comprising the sequence TTTTTTTTTTTTAATGATGGCGTAG (SEQ ID NO:43); a second probe comprising the sequence TTTTTTTTTTTAGTAGTTGGAGCTG (SEQ ID NO:51); a third probe comprising the sequence TTTTTTTTTTTAATTGTGGCGTAGG (SEQ ID NO:65); a fourth probe comprising the sequence TTTTTTTTTAGCTCAGTGATTTTAG (SEQ ID NO:88); a fifth probe comprising the sequence TTTTTTTTTTTTTTTCCTGCTTAGTG (SEQ ID NO: 104); a sixth probe comprising the sequence TTTTTTTTTTTTTGATGCACGTC (SEQ ID NO:112); a seventh probe comprising the sequence TTTTTTAATTGAGAAATCTCGATGGAG (SEQ ID NO:78); an eighth probe comprising the sequence TTTTTTAGATCAAAGTGCTGGCCTCCG (SEQ ID NO:120); a ninth probe comprising the sequence TTTTTTTTTTTTTTAAACATCTCCGAAAGCC (SEQ ID NO:135); a tenth probe comprising the sequence TTTTTTTTTTTCAAGACATCTCCGA (SEQ ID NO:145); an eleventh probe comprising the sequence TTTTTTTTTTTGAGATGCATGATGA (SEQ ID NO:352); a twelfth probe comprising the sequence TTTTTTTTTTTACAGGAGGCTGCC (SEQ ID NO:470); a thirteenth probe comprising the sequence TTTTTTTTTTTACCAGGAGGGAGCCG (SEQ ID NO:474); a fourteenth probe comprising the sequence TTTTTTTTTATGGCCATCTTGG (SEQ ID NO:422); a fifteenth probe comprising the sequence TTTTTTTTTTTTTGATGGCCCGCGTG (SEQ ID NO:440); a sixteenth probe comprising the sequence TTTTTTTTTTTTCCCATCACGTGT (SEQ ID NO:448); a seventeenth probe comprising the sequence TTTTTTTTTTTGACGGTAACCCCC (SEQ ID NO:456); an eighteenth probe comprising the sequence TTTTTTTATTTTGGGCGGGCC (SEQ ID NO:152); a nineteenth probe comprising the sequence TTTTTTTTTTTTCTGTAGGGAAGTAC (SEQ ID NO:373); a twentieth probe comprising the sequence TTTTTAAATTTCTTACCCAGAATCA (SEQ ID NO:392); a twenty-first probe comprising the sequence TTTTTAAGCATACGTGATGGCT (SEQ ID NO:409); a twenty-second probe comprising the sequence TTTTTTTTTTGGGAAAGGACCTAAAG (SEQ ID NO:166); a twenty-third probe comprising the sequence TTTTTTTTTTTTCCGCCGGAAGCACCAGGA (SEQ ID NO:180); a twenty-fourth probe comprising the sequence TTTTTTTTTTTTTTGTGTACCGCCGGAAGC (SEQ ID NO:186); a twenty-fifth probe comprising the sequence TTTTTTTTTTTTTCGAAAAAAACAGCCAA (SEQ ID NO:192); a twenty-sixth probe comprising the sequence TTTTTTTTTTTACTGACGCTCCACCGAAA (SEQ ID NO:202); a twenty-seventh probe comprising the sequence TTTTTTTTTTTTGACGCTCCACCGAAA (SEQ ID NO:214); a twenty-eighth probe comprising the sequence TTTTTTTTTTTTAGCGCCTTCCAGCTGGTTGGA (SEQ ID NO:222); a twenty-ninth probe comprising the sequence TTTTTTTTTTTGATTTTTGGATACCA (SEQ ID NO:240); a thirtieth probe comprising the sequence TTTTTTTTTTTCCACTGTGCGACGAGCTGT (SEQ ID NO:250); a thirty-first probe comprising the sequence TTTTTTTTTGGATCCAAAGTGGGAATT (SEQ ID NO:258); a thirty-second probe comprising the sequence TTTTTTTTTTCAAGAGGATCCAAA (SEQ ID NO:488); a thirty-third probe comprising the sequence TTTTTTTTACAAGAGTTAAAAAGGAGGA (SEQ ID NO:494); a thirty-fourth probe comprising the sequence TTTTTATTAAGTGCACAAACAGGAGG (SEQ ID NO:504); a thirty-fifth probe comprising the sequence TTTTTTTTTTACTAACAGCACATCTGGAGA (SEQ ID NO:270); a thirty-sixth probe comprising the sequence TTTTTTTTTTAGATCAGTTTCCTAATTC (SEQ ID NO:278); (b) a plurality of primer pairs, the plurality of primer pairs comprising a first primer pair comprising the sequences GTACTGGTGGAGTATTTGATAGTG (SEQ ID NO:1) and CGTCAAGGCACTCTTGCCTAC (SEQ ID NO:2); a second primer pair comprising the sequences CAATTTCTACAAGAGATCCTCTCTCT (SEQ ID NO:5) and CTCCATTTTAGCACTTACCTGTGAC (SEQ ID NO:6); a third primer pair comprising the sequences ACCCTAGCCTTAGATAAAACTGAGC (SEQ ID NO:7) and TTTGTTGTCCAGCCACCATGA (SEQ ID NO:8); a fourth primer pair comprising the sequences ATAGCCTCAATTCTTACCATCCACAAAATG (SEQ ID NO:9) and CAGATATATTTCTTCATGAAGACCTCACAGTAA (SEQ ID NO:10); a fifth primer pair comprising the sequences CTTGTGGAGCCTCTTACACCC (SEQ ID NO:11) and TGCCGAACGCACCGGA (SEQ ID NO:12); a sixth primer pair comprising the sequences GCCAGTTAACGTCTTCCTTCTC (SEQ ID NO:13) and ATCGAGGATTTCCTTGTTGGCTT (SEQ ID NO:14); a seventh primer pair comprising the sequences CCTCCACCGTGCAGATCATC (SEQ ID NO: 15) and TTCCCTGATTACCTTTGCGAT (SEQ ID NO:16); an eighth primer pair comprising the sequences CCACACTGACGTGCCTCT (SEQ ID NO:511) and GCACACGTAGGGGTTGTCCAAGA (SEQ ID NO:512); a ninth primer pair comprising the sequences CCACACTGACGTGCCTCT (SEQ ID NO:513) and GTACACGCTGGCCACGCCG (SEQ ID NO:514); a tenth primer pair comprising the sequences CCACACTGACGTGCCTCT (SEQ ID NO:515) and CAGGCGGCACACGTGAT (SEQ ID NO:516); an eleventh primer pair comprising the sequences CCACACTGACGTGCCTCT (SEQ ID NO:517) and AGGCGGCACACGTGCGGGTTAC (SEQ ID NO:518); a twelfth primer pair comprising the sequences GGAGGACCGTCGCTTGG (SEQ ID NO:17) and TCTTTCTCTTCCGCACCCAG (SEQ ID NO:18); a thirteenth primer pair comprising the sequences GAGGGTCTGACGGGTAGAGTG (SEQ ID NO:380) and TGGCCGCCAGGTCTTGATGTA (SEQ ID NO:381); a fourteenth primer pair comprising the sequences CTTGATGAGCAGCAGCGAAA (SEQ ID NO:397) and CCTTCAGTTCTCCCACCTTCTG (SEQ ID NO:398); a fifteenth primer pair comprising the sequences ATGGCTGTGGTTTGTGATGGT (SEQ ID NO:414) and ACACCAGCCATCACGTAAGACA (SEQ ID NO:415); a sixteenth primer pair comprising the sequences AGTTGGGGTTGTAGTCGGTCAT (SEQ ID NO:363) and TATGGAGCAAAACTACTGTAGAGCC (SEQ ID NO:357); a seventeenth primer pair comprising the sequences AGTTGGGGTTGTAGTCGGTCAT (SEQ ID NO:363) and CCAGCTACATCACACACCTTGACT (SEQ ID NO:358); an eighteenth primer pair comprising the sequences AGTTGGGGTTGTAGTCGGTCAT (SEQ ID NO:363) and TAATACCAAAAGTTACCAAAACTGCA (SEQ ID NO:359); a nineteenth primer pair comprising the sequences GGAGTGCCATCGCTGTTTGAAATGAGCAGGCACT (SEQ ID NO:19); a twentieth primer pair comprising the sequences GGAGTGCCATCGCTGTTTGAAATGAGCAGGCACT (SEQ ID NO:19); a twenty-first primer pair comprising the sequences TACAGCCCTGGATATTCTTAGTAGCGC (SEQ ID NO:20); a twenty-second primer pair comprising the sequences TACAGCCCTGGATATTCTTAGTAGCGC (SEQ ID NO:20); a twenty-third primer pair comprising the sequences GTGATCGCACAGTAGGACAGCGGCTGCGATC (SEQ ID NO:26) and TTTCTGGTGCTATGAGGAAATGACCAACCACCAGA (SEQ ID NO:23); a twenty-fourth primer pair comprising the sequences CTCTAGGAGATATCATTCCAAATTCGCCTTCTCCTAG (SEQ ID NO:27) and TTTCTGGTGCTATGAGGAAATGACCAACCACCAGA (SEQ ID NO:23); a twenty-fifth primer pair comprising the sequences CTCTAGGAGATATCATTCCAAATTCGCCTTCTCCTAG (SEQ ID NO:27) and AAGGAGTTAGCAGCATGTCAGC (SEQ ID NO:519); a twenty-sixth primer pair comprising the sequences CTCTAGGAGATATCATTCCAAATTCGCCTTCTCCTAG (SEQ ID NO:27) and AACTTCAGACTTTACACAACCTGC (SEQ ID NO:520); a twenty-seventh primer pair comprising the sequences CTCTAGGAGATATCATTCCAAATTCGCCTTCTCCTAG (SEQ ID NO:27) and ATTGATTCTGATGACACCGGA (SEQ ID NO:521); a twenty-eighth primer pair comprising the sequences GGACGAAAATCCAGACCCCAAAAGGTGTTTCGT (SEQ ID NO:32) and AGAAGACGTGACAGGAACTGGAGGACCCGTCTT (SEQ ID NO:30); a twenty-ninth primer pair comprising the sequences GACAGTATTTTGCAGTAATGGACTGGATATATCAGA (SEQ ID NO:29) and GAATTTCACAGGATTGATTGCTGGTGTTGTCTC (SEQ ID NO:28); and (c) a plurality of blocking nucleic acids, the plurality of blocking nucleic acids comprising a first blocking nucleic acid comprising the sequence TTGGAGCTGGTGGCGTA(invdT)ₙ, wherein n is 1, 2, or 3 (SEQ ID NO:282); a second blocking nucleic acid comprising the sequence TCTCTGAAATCACTGAGCAGG(invdT)ₙ, wherein n is 1, 2, or 3 (SEQ ID NO:294); a third blocking nucleic acid comprising the sequence CCACCATGATGTGCAT(invdT)ₙ, wherein n is 1, 2, or 3 (SEQ ID NO:297); a fourth blocking nucleic acid comprising the sequence GAGATTTCACTGTAGC(invdT)ₙ, wherein n is 1, 2, or 3 (SEQ ID NO:305); a fifth blocking nucleic acid comprising the sequence CGCACCGGAGCCCAGCAC (invdT)ₙ, wherein n is 1, 2, or 3 (SEQ ID NO:309); a sixth blocking nucleic acid comprising the sequence GTTGCTTCTCTTAATTCC(invdT)ₙ, wherein n is 1, 2, or 3 (SEQ ID NO:313); a seventh blocking nucleic acid comprising the sequence CTCATCACGCAGC(invdT)ₙ, wherein n is 1, 2, or 3 (SEQ ID NO:320); an eighth blocking nucleic acid comprising the sequence CCAGCAGTTTGGCCAGCCCT(invdT)ₙ, wherein n is 1, 2, or 3 (SEQ ID NO:325); a ninth blocking nucleic acid comprising the sequence; GATGTACTCCCCTACA (invdT)ₙ, wherein n is 1, 2, or 3 (SEQ ID NO:386); a tenth blocking nucleic acid comprising the sequence CACCTTCTGCTTCTGGG (invdT)ₙ, wherein n is 1, 2, or 3 (SEQ ID NO:401).

In another aspect, provided herein is a kit comprising: (a) a plurality of probes, wherein each probe of the plurality is coupled to a microcarrier that has a unique identifier corresponding to the probe coupled thereto, the plurality of probes comprising a first probe comprising the sequence TTTTTTTTTTTATGGAGCTGATGGC (SEQ ID NO:45); a second probe comprising the sequence TTTTTTTTTTTATGGAGCTGTTGGC (SEQ ID NO:53); a third probe comprising the sequence TTTTTTTTTTTAAGGAGCTTGTGGC (SEQ ID NO:63); a fourth probe comprising the sequence TTTTTTTTTTTCTCAGTGATTTTAGA (SEQ ID NO:96); a fifth probe comprising the sequence TTTTTTTTTTTTTCTCCTGCTTAGT (SEQ ID NO:100); a sixth probe comprising the sequence TTTTTTTTTTTTAATGATGCACGTC (SEQ ID NO:116); a seventh probe comprising the sequence TTTTTTAATTTCTAGCTACAGAGAAAT (SEQ ID NO:86); an eighth probe comprising the sequence TTTTTTTTTTCAAAGTGCTGGCCTC (SEQ ID NO:118); a ninth probe comprising the sequence TTTTTTTTTAATCAAAACATCTCCG (SEQ ID NO:127); a tenth probe comprising the sequence TTTTTTTTAATCAAGACATCTCCGA (SEQ ID NO:137); an eleventh probe comprising the sequence TTTTTTTTTTGAGATGCATGATGAG (SEQ ID NO:353); a twelfth probe comprising the sequence TTTTTTTTTTTACCAGGAGGCTGCC (SEQ ID NO:468); a thirteenth probe comprising the sequence TTTTTTTTTTTACCAGGAGGGAGCC (SEQ ID NO:472); a fourteenth probe comprising the sequence TTTTTTTTTTAGGCCATCTTGGA (SEQ ID NO:424); a fifteenth probe comprising the sequence TTTTTTTTTTTGTGATGGCCGGCGT (SEQ ID NO:436); a sixteenth probe comprising the sequence TTTTTTTTTTTAACCCCCATCACGT (SEQ ID NO:442); a seventeenth probe comprising the sequence TTTTTTTTTTTCGTGGACGGTAACC (SEQ ID NO:454); an eighteenth probe comprising the sequence TTTTTTTAAAAAAGCGGGCCAAACT (SEQ ID NO:156); a nineteenth probe comprising the sequence TTTTTTTTACGTCTGTAGGGAAGTA (SEQ ID NO:379); a twentieth probe comprising the sequence TTTTTAAATTTACCCAGAATCAGAA (SEQ ID NO:388); a twenty-first probe comprising the sequence TTTTTTTTTATACGTGATGTCTTAC (SEQ ID NO:405); a twenty-second probe comprising the sequence TTTTTTTTTTCCTAAAGTGTACCGC (SEQ ID NO:164); a twenty-third probe comprising the sequence TTTTTTTTTTTTTATTGTACTTGTACCGCC (SEQ ID NO:176); a twenty-fourth probe comprising the sequence TTTTTTTTTTTTTTTCAACCAAGTGTACCG (SEQ ID NO:188); a twenty-fifth probe comprising the sequence TTTTTTTTTTTTTTACAGCCAAGTGTACCG (SEQ ID NO:200); a twenty-sixth probe comprising the sequence TTTTTTTTTTTCACCGAAAGCTGGAGTCCC (SEQ ID NO:210); a twenty-seventh probe comprising the sequence TTTTTTTTTTTTCCGAAAGATGATTTT (SEQ ID NO:212); a twenty-eighth probe comprising the sequence TTTTTTTTTTTTCTGGTTGGAGCTGGAGTCCC (SEQ ID NO:224); a twenty-ninth probe comprising the sequence TTTTTTTTTTTGGTTGGAGATGATTTTT (SEQ ID NO:236); a thirtieth probe comprising the sequence TTTTTTTTTTTGATGTAAAGATCCACTGTG (SEQ ID NO:248); a thirty-first probe comprising the sequence TTTTTTTTTATGATGTAAAGGAGGATCC (SEQ ID NO:260); a thirty-second probe comprising the sequence TTTTTTTTTGTATCTCTCAAGAGGATCCAA (SEQ ID NO:484); a thirty-third probe comprising the sequence TTATTATTAAGAGTTAAAAAGGAGGATC (SEQ ID NO:811); a thirty-fourth probe comprising the sequence TATTATTATGTGCACAAACAGGAGGATC (SEQ ID NO:506); a thirty-fifth probe comprising the sequence TTTTTTTTTTAGGGCCCAGACACTAACAGC (SEQ ID NO:268); a thirty-sixth probe comprising the sequence TTTTTTTTTTAAATTAAAGATCAGTTTC (SEQ ID NO:276); (b) a plurality of primer pairs, the plurality of primer pairs comprising a first primer pair comprising the sequences GTACTGGTGGAGTATTTGATAGTG (SEQ ID NO:1) and CGTCAAGGCACTCTTGCCTAC (SEQ ID NO:2); a second primer pair comprising the sequences CAATTTCTACAAGAGATCCTCTCTCT (SEQ ID NO:5) and CTCCATTTTAGCACTTACCTGTGAC (SEQ ID NO:6); a third primer pair comprising the sequences ACCCTAGCCTTAGATAAAACTGAGC (SEQ ID NO:7) and TTTGTTGTCCAGCCACCATGA (SEQ ID NO:8); a fourth primer pair comprising the sequences ATAGCCTCAATTCTTACCATCCACAAAATG (SEQ ID NO:9) and CAGATATATTTCTTCATGAAGACCTCACAGTAA (SEQ ID NO:10); a fifth primer pair comprising the sequences CTTGTGGAGCCTCTTACACCC (SEQ ID NO:11) and TGCCGAACGCACCGGA (SEQ ID NO:12); a sixth primer pair comprising the sequences GCCAGTTAACGTCTTCCTTCTC (SEQ ID NO:13) and ATCGAGGATTTCCTTGTTGGCTT (SEQ ID NO: 14); a seventh primer pair comprising the sequences CCTCCACCGTGCAGATCATC (SEQ ID NO:15) and TTCCCTGATTACCTTTGCGAT (SEQ ID NO:16); an eighth primer pair comprising the sequences CCACACTGACGTGCCTCT (SEQ ID NO:511) and GCACACGTAGGGGTTGTCCAAGA (SEQ ID NO: 512); a ninth primer pair comprising the sequences CCACACTGACGTGCCTCT (SEQ ID NO:513) and GTACACGCTGGCCACGCCG (SEQ ID NO:514); a tenth primer pair comprising the sequences CCACACTGACGTGCCTCT (SEQ ID NO:515) and CAGGCGGCACACGTGAT (SEQ ID NO:516); an eleventh primer pair comprising the sequences CCACACTGACGTGCCTCT (SEQ ID NO:517) and AGGCGGCACACGTGCGGGTTAC (SEQ ID NO:518); a twelfth primer pair comprising the sequences GGAGGACCGTCGCTTGG (SEQ ID NO:17) and TCTTTCTCTTCCGCACCCAG (SEQ ID NO:18); a thirteenth primer pair comprising the sequences GAGGGTCTGACGGGTAGAGTG (SEQ ID NO:380) and TGGCCGCCAGGTCTTGATGTA (SEQ ID NO:381); a fourteenth primer pair comprising the sequences CTTGATGAGCAGCAGCGAAA (SEQ ID NO:397) and CCTTCAGTTCTCCCACCTTCTG (SEQ ID NO:398); a fifteenth primer pair comprising the sequences ATGGCTGTGGTTTGTGATGGT (SEQ ID NO:414) and ACACCAGCCATCACGTAAGACA (SEQ ID NO:415); a sixteenth primer pair comprising the sequences AGTTGGGGTTGTAGTCGGTCAT (SEQ ID NO:363) and TATGGAGCAAAACTACTGTAGAGCC (SEQ ID NO:357); a seventeenth primer pair comprising the sequences AGTTGGGGTTGTAGTCGGTCAT (SEQ ID NO:363) and CCAGCTACATCACACACCTTGACT (SEQ ID NO:358); an eighteenth primer pair comprising the sequences AGTTGGGGTTGTAGTCGGTCAT (SEQ ID NO:363) and TAATACCAAAAGTTACCAAAACTGCA (SEQ ID NO:359); a nineteenth primer pair comprising the sequences GGAGTGCCATCGCTGTTTGAAATGAGCAGGCACT (SEQ ID NO:19); a twentieth primer pair comprising the sequences GGAGTGCCATCGCTGTTTGAAATGAGCAGGCACT (SEQ ID NO:19); a twenty-first primer pair comprising the sequences TACAGCCCTGGATATTCTTAGTAGCGC (SEQ ID NO:20); a twenty-second primer pair comprising the sequences TACAGCCCTGGATATTCTTAGTAGCGC (SEQ ID NO:20); a twenty-third primer pair comprising the sequences GTGATCGCACAGTAGGACAGCGGCTGCGATC (SEQ ID NO:26) and TTTCTGGTGCTATGAGGAAATGACCAACCACCAGA (SEQ ID NO:23); a twenty-fourth primer pair comprising the sequences CTCTAGGAGATATCATTCCAAATTCGCCTTCTCCTAG (SEQ ID NO:27) and TTTCTGGTGCTATGAGGAAATGACCAACCACCAGA (SEQ ID NO:23); a twenty-fifth primer pair comprising the sequences CTCTAGGAGATATCATTCCAAATTCGCCTTCTCCTAG (SEQ ID NO:27) and AAGGAGTTAGCAGCATGTCAGC (SEQ ID NO:519); a twenty-sixth primer pair comprising the sequences CTCTAGGAGATATCATTCCAAATTCGCCTTCTCCTAG (SEQ ID NO:27) and AACTTCAGACTTTACACAACCTGC (SEQ ID NO:520); a twenty-seventh primer pair comprising the sequences CTCTAGGAGATATCATTCCAAATTCGCCTTCTCCTAG (SEQ ID NO:27) and ATTGATTCTGATGACACCGGA (SEQ ID NO:521); a twenty-eighth primer pair comprising the sequences GGACGAAAATCCAGACCCCAAAAGGTGTTTCGT (SEQ ID NO:32) and AGAAGACGTGACAGGAACTGGAGGACCCGTCTT (SEQ ID NO:30); a twenty-ninth primer pair comprising the sequences GACAGTATTTTGCAGTAATGGACTGGATATATCAGA (SEQ ID NO:29) and GAATTTCACAGGATTGATTGCTGGTGTTGTCTC (SEQ ID NO:28); and (c) a plurality of blocking nucleic acids, the plurality of blocking nucleic acids comprising a first blocking nucleic acid comprising the sequence TACGCCACCAGCT(invdT)ₙ, wherein n is 1, 2, or 3 (SEQ ID NO:281); a second blocking nucleic acid comprising the sequence TCTCTGAAATCACTGAGCAGG(invdT)ₙ, wherein n is 1, 2, or 3 (SEQ ID NO:293); a third blocking nucleic acid comprising the sequence CACCATGATGTGCAT(invdT)ₙ, wherein n is 1, 2, or 3 (SEQ ID NO:296); a fourth blocking nucleic acid comprising the sequence GAGATTTCACTGTAGC(invdT)ₙ, wherein n is 1, 2, or 3 (SEQ ID NO:303); a fifth blocking nucleic acid comprising the sequence GAGCCCAGCAC (invdT)ₙ, wherein n is 1, 2, or 3 (SEQ ID NO:308); a sixth blocking nucleic acid comprising the sequence CGGAGATGTTGCTTCTCTTAATTCC(invdT)ₙ, wherein n is 1, 2, or 3 (SEQ ID NO:311); a seventh blocking nucleic acid comprising the sequence CATCACGCAGCTCATG(invdT)ₙ, wherein n is 1, 2, or 3 (SEQ ID NO:316); an eighth blocking nucleic acid comprising the sequence CCAGCAGTTTGGCCAGCCCT(invdT)ₙ, wherein n is 1, 2, or 3 (SEQ ID NO:321); a ninth blocking nucleic acid comprising the sequence TGTACTCCCCTACA (invdT)ₙ, wherein n is 1, 2, or 3 (SEQ ID NO:382); a tenth blocking nucleic acid comprising the sequence TCTGCTTCTGGGTAAG (invdT)ₙ, wherein n is 1, 2, or 3 (SEQ ID NO:399).

It is to be understood that one, some, or all of the properties of the various embodiments described herein may be combined to form other embodiments of the present invention. These and other aspects of the invention will become apparent to one of skill in the art.

### BRIEF DESCRIPTION OF THE DRAWINGS

FIGS. 1A & 1B show two views of an exemplary microcarrier.

FIGS. 1C & 1D show an exemplary assay for DNA detection using an exemplary microcarrier.

FIG. 2A shows three examples of microcarriers, each having a unique analog code.

FIG. 2B shows examples of microcarriers with a unique analog code, in accordance with some embodiments.

FIG. 2C shows an example of a microcarrier with a unique analog code, in accordance with some embodiments.

FIG. 3 shows a flowchart illustrating an exemplary method for detecting the presence of DNA mutation(s) and RNA variant(s), in accordance with some embodiments.

FIGS. 4 & 5 illustrate an exemplary scheme for preferentially amplifying and detecting mutant (FIG. 5) over wild-type (FIG. 4) loci corresponding to a DNA mutation of interest, in accordance with some embodiments. Solid horizontal lines indicate amplified DNA sequences, dashed horizontal lines indicate primer/probe/blocking nucleic acid (NA) sequences, and vertical lines indicate Watson-Crick base pairing.

FIG. 6 shows a flowchart illustrating an exemplary protocol for isolating RNA and cell-free DNA (cfDNA) from a blood sample.

FIGS. 7A-7C show the results of multiplex detection of DNA mutations. Values reflect the fluorescence signal (in arbitrary units, AU) obtained for each pairwise combination of amplified DNA specific for each indicated DNA mutation (columns) and microcarrier-coupled probe specific for each indicated DNA mutation (rows).

FIGS. 8A & 8B show the results of multiplex detection of RNA variants. Values reflect the fluorescence signal (in arbitrary units, AU) obtained for each pairwise combination of RNA sample specific for each indicated RNA variant (columns) and microcarrier-coupled probe specific for each indicated RNA variant (rows).

FIGS. 9A & 9B show the results of multiplex detection of RNA and DNA mutations from patient samples. In FIG. 9A, RNA was obtained from formalin-fixed, paraffin-embedded (FFPE) samples, and selected mutations were detected by next-generation sequencing (NGS), as compared to the microcarrier approach described herein (LCP). In FIG. 9B, DNA was obtained from pleural effusion or cfDNA in serum samples, and selected mutations were detected by ddPCR or next-generation sequencing (NGS), as compared to the microcarrier approach described herein (LCP).

FIGS. 10A-10C show comparisons between the microcarrier approach described herein (LCP) and other mutation detection techniques in detecting DNA or RNA mutations in tissue samples or liquid biopsies (blood samples) obtained from patients with stage I or II lung cancer. Shown are the results obtained using DNA or RNA from liquid biopsies (FIG. 10A), DNA from tissue samples (FIG. 10B), or RNA from tissue samples (FIG. 10C). These results demonstrate the versatility of the LCP approach in detecting a variety of cancer-associated mutations from different samples in DNA or RNA.

### DETAILED DESCRIPTION

In one aspect, provided herein are methods for detecting the presence of DNA mutations in the KRAS, NRAS, PIK3CA, BRAF, EGFR, AKT1, MEK1, and HER2 genes and/or RNA mutations in the ALK, ROS, RET, NTRK1, and cMET genes.

In some embodiments, the methods include isolating DNA from a sample; amplifying the isolated DNA by polymerase chain reaction (PCR) using primer pairs specific for the loci of one or more DNA mutations in each of the KRAS, NRAS, PIK3CA, BRAF, EGFR, AKT1, MEK1, and HER2 genes; hybridizing the amplified DNA with at least seven probes, said at least seven probes comprising one or more probes specific for a DNA mutation in each of the KRAS, NRAS, PIK3CA, BRAF, EGFR, AKT1, MEK1, and HER2 genes, wherein each of said at least seven probes is coupled to a microcarrier, and wherein each of the microcarriers comprises an identifier corresponding to the probe coupled thereto; detecting presence or absence of hybridization of the amplified DNA with said at least seven probes, wherein hybridization between the amplified DNA and one of the probes indicates the presence of the DNA mutation corresponding to the probe; detecting the identifiers of the microcarriers; and correlating the detected identifiers of the microcarriers with the detected presence or absence of hybridization of the amplified DNA to the corresponding probes of the microcarriers.

In some embodiments, the methods include isolating RNA from a sample; amplifying DNA from the isolated RNA by reverse transcription-polymerase chain reaction (RT-PCR), wherein amplifying the DNA comprises: generating cDNA specific for each of the ALK, ROS, RET, NTRK1, and cMET genes from the isolated RNA using a first primer specific for each of the ALK, ROS, RET, NTRK1, and cMET genes, the isolated RNA, and a reverse transcriptase, and amplifying DNA specific for each of the ALK, ROS, RET, NTRK1, and cMET genes by polymerase chain reaction (PCR) using the cDNA, a DNA polymerase, the first primer, and a second primer specific for each of the ALK, ROS, RET, NTRK1, and cMET genes that binds to a strand of the cDNA opposite the corresponding first primer and promotes strand extension in a direction opposite that promoted by the corresponding first primer; hybridizing the amplified DNA with at least five probes, said at least five probes comprising one or more probes specific for a mutation in each of the ALK, ROS, RET, NTRK1, and cMET genes, wherein each of said at least five probes is coupled to a microcarrier, and wherein each of the microcarriers comprises an identifier corresponding to the probe coupled thereto; detecting presence or absence of hybridization of the amplified DNA with said at least five probes, wherein hybridization between the amplified DNA and one of the probes indicates the presence of the mutation corresponding to the probe; detecting the identifiers of the microcarriers; and correlating the detected identifiers of the microcarriers with the detected presence or absence of hybridization of the amplified DNA to the corresponding probes of the microcarriers.

In some embodiments, the methods include isolating DNA and RNA from a sample; amplifying the isolated DNA by polymerase chain reaction (PCR) using primer pairs specific for the loci of one or more DNA mutations in each of the KRAS, NRAS, PIK3CA, BRAF, EGFR, AKT1, MEK1, and HER2 genes; amplifying DNA from the isolated RNA by reverse transcription-polymerase chain reaction (RT-PCR), wherein amplifying the DNA from the isolated RNA comprises: generating cDNA specific for each of the ALK, ROS, RET, NTRK1, and cMET genes from the isolated RNA using a first primer specific for each of the ALK, ROS, RET, NTRK1, and cMET genes, the isolated RNA, and a reverse transcriptase, and amplifying DNA specific for each of the ALK, ROS, RET, NTRK1, and cMET genes by polymerase chain reaction (PCR) using the cDNA, a DNA polymerase, the first primer, and a second primer specific for each of the ALK, ROS, RET, NTRK1, and cMET genes that binds to a strand of the cDNA opposite the corresponding first primer and promotes strand extension in a direction opposite that promoted by the corresponding first primer; hybridizing the DNA amplified by PCR from the isolated DNA with at least seven probes, said at least seven probes comprising one or more probes specific for a mutation in each of the KRAS, NRAS, PIK3CA, BRAF, EGFR, AKT1, MEK1, and HER2 genes, wherein each of said at least seven probes is coupled to a microcarrier, and wherein each of the microcarriers comprises an identifier corresponding to the probe coupled thereto; detecting presence or absence of hybridization of the DNA amplified by PCR from the isolated DNA with said at least seven probes, wherein hybridization between the amplified DNA and one of the probes indicates the presence of the mutation corresponding to the probe; hybridizing the DNA amplified by RT-PCR from the isolated RNA with at least five probes, said at least five probes comprising one or more probes specific for a mutation in each of the ALK, ROS, RET, NTRK1, and cMET genes, wherein each of said at least five probes is coupled to a microcarrier, and wherein each of the microcarriers comprises an identifier corresponding to the probe coupled thereto; detecting presence or absence of hybridization of the DNA amplified by RT-PCR from the isolated RNA with said at least five probes, wherein hybridization between the amplified DNA and one of the probes indicates the presence of the mutation corresponding to the probe; detecting the identifiers of the microcarriers; and correlating the detected identifiers of the microcarriers with the presence or absence of hybridization of the amplified DNA to the corresponding probes of the microcarriers.

In another aspect, provided herein are kits for performing any of the methods described herein. In some embodiments, the kits include microcarriers, probe sequences, primers, and/or blocking nucleic acids, e.g., as described herein.

### I. General Techniques

The practice of the techniques described herein will employ, unless otherwise indicated, conventional techniques in polymer technology, microfabrication, micro-electro-mechanical systems (MEMS) fabrication, photolithography, microfluidics, organic chemistry, biochemistry, oligonucleotide synthesis and modification, bioconjugate chemistry, nucleic acid hybridization, molecular biology, microbiology, genetics, recombinant DNA, and related fields as are within the skill of the art. The techniques are described in the references cited herein and are fully explained in the literature.

For molecular biology and recombinant DNA techniques, see, for example, (Maniatis, T. et al. (1982), Molecular Cloning: A Laboratory Manual, Cold Spring Harbor; Ausubel, F. M. (1987), Current Protocols in Molecular Biology, Greene Pub. Associates and Wiley-Interscience; Ausubel, F. M. (1989), Short Protocols in Molecular Biology: A Compendium of Methods from Current Protocols in Molecular Biology, Greene Pub. Associates and Wiley-Interscience; Sambrook, J. et al. (1989), Molecular Cloning: A Laboratory Manual, Cold Spring Harbor; Innis, M. A. (1990), PCR Protocols: A Guide to Methods and Applications, Academic Press; Ausubel, F. M. (1992), Short Protocols in Molecular Biology: A Compendium of Methods from Current Protocols in Molecular Biology, Greene Pub. Associates; Ausubel, F. M. (1995), Short Protocols in Molecular Biology: A Compendium of Methods from Current Protocols in Molecular Biology, Greene Pub. Associates; Innis, M. A. et al. (1995), PCR Strategies, Academic Press; Ausubel, F. M. (1999), Short Protocols in Molecular Biology: A Compendium of Methods from Current Protocols in Molecular Biology, Wiley, and annual updates.

For DNA synthesis techniques and nucleic acids chemistry, see for example, Gait, M. J. (1990), Oligonucleotide Synthesis: A Practical Approach, IRL Press; Eckstein, F. (1991), Oligonucleotides and Analogues: A Practical Approach, IRL Press; Adams, R. L. et al. (1992), The Biochemistry of the Nucleic Acids, Chapman & Hall; Shabarova, Z. et al. (1994), Advanced Organic Chemistry of Nucleic Acids, Weinheim; Blackburn, G. M. et al. (1996), Nucleic Acids in Chemistry and Biology, Oxford University Press; Hermanson, G. T. (1996), Bioconjugate Techniques, Academic Press).

For microfabrication, see for example, (Campbell, S. A. (1996), The Science and Engineering of Microelectronic Fabrication, Oxford University Press; Zaut, P. V. (1996), Microarray Fabrication: a Practical Guide to Semiconductor Processing, Semiconductor Services; Madou, M. J. (1997), Fundamentals of Microfabrication, CRC Press; Rai-Choudhury, P. (1997). Handbook of Microlithography, Micromachining, & Microfabrication: Microlithography).

### II. Definitions

Before describing the invention in detail, it is to be understood that this invention is not limited to particular compositions or biological systems, which can, of course, vary. It is also to be understood that the terminology used herein is for the purpose of describing particular embodiments only, and is not intended to be limiting.

The term “microcarrier” as used herein may refer to a physical substrate onto which a capture agent or probe may be coupled. A microcarrier of the present disclosure may take any suitable geometric form or shape. In some embodiments, the microcarrier may be disc-shaped. Typically the form or shape of a microcarrier will include at least one dimension on the order of 10⁻⁴ to 10⁻⁷ m (hence the prefix “micro”).

The term “polymer” as used herein may refer to any macromolecular structure comprising repeated monomers. A polymer may be natural (e.g., found in nature) or synthetic (e.g., man-made, such as a polymer composed of non-natural monomer(s) and/or polymerized in a configuration or combination not found in nature).

The terms “substantially transparent” and “substantially non-transparent” as used herein may refer to the ability of light (e.g., of a particular wavelength, such as infrared, visible, UV, and so forth) to pass through a substrate, such as a polymer layer. A substantially transparent polymer may refer to one that is transparent, translucent, and/or pervious to light, whereas a substantially non-transparent polymer may refer to one that reflects and/or absorbs light. It is to be appreciated that whether a material is substantially transparent or substantially non-transparent may depend upon the wavelength and/or intensity of light illuminating the material, as well as the means detecting the light traveling through the material (or a decrease or absence thereof). In some embodiments, a substantially non-transparent material causes a perceptible decrease in transmitted light as compared to the surrounding material or image field, e.g., as imaged by light microscopy (e.g., bright field, dark field, phase contrast, differential interference contrast (DIC), Nomarski interference contrast (NIC), Nomarski, Hoffman modulation contrast (HMC), or fluorescence microscopy). In some embodiments, a substantially transparent material allows a perceptible amount of transmitted light to pass through the material, e.g., as imaged by light microscopy (e.g., bright field, dark field, phase contrast, differential interference contrast (DIC), Nomarski interference contrast (NIC), Nomarski, Hoffman modulation contrast (HMC), or fluorescence microscopy).

The term “analog code” as used herein may refer to any code in which the encoded information is represented in a non-quantized and/or non-discrete manner, e.g., as opposed to a digital code. For example, a digital code is sampled at discrete positions for a limited set of values (e.g., 0/1 type values), whereas an analog code may be sampled at a greater range of positions (or as a continuous whole) and/or may contain a wider set of values (e.g., shapes). In some embodiments, an analog code may be read or decoded using one or more analog shape recognition techniques.

As used herein, “sample” refers to a composition containing a material, such as a molecule, to be detected. In one embodiment, the sample is a “biological sample” (i.e., any material obtained from a living source (e.g. human, animal, plant, bacteria, fungi, protist, virus)). The biological sample can be in any form, including solid materials (e.g. tissue, cell pellets, biopsies, FFPE samples, etc.) and biological fluids (e.g. urine, blood or plasma, stool, saliva, lymph, tears, sweat, prostatic fluid, seminal fluid, semen, bile, mucus, pleural effusion, amniotic fluid and mouth wash (containing buccal cells)). Solid materials typically are mixed with a fluid. Sample can also refer to an environmental sample such as water, air, soil, or any other environmental source.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

### III. Methods of Detecting DNA and/or RNA Mutations

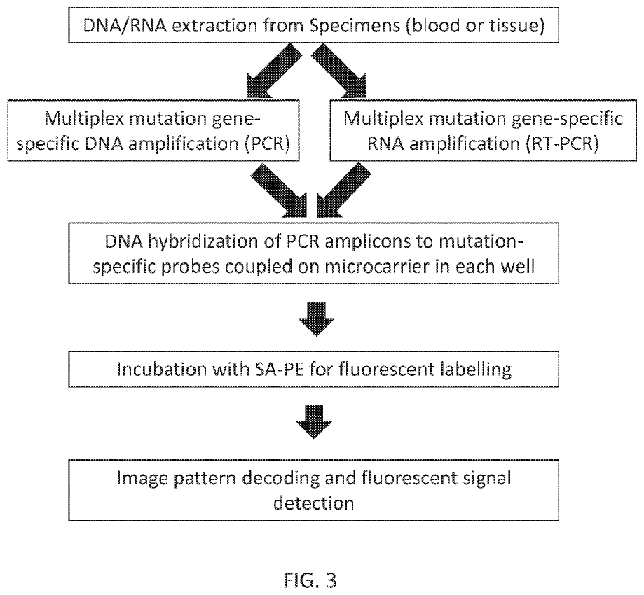

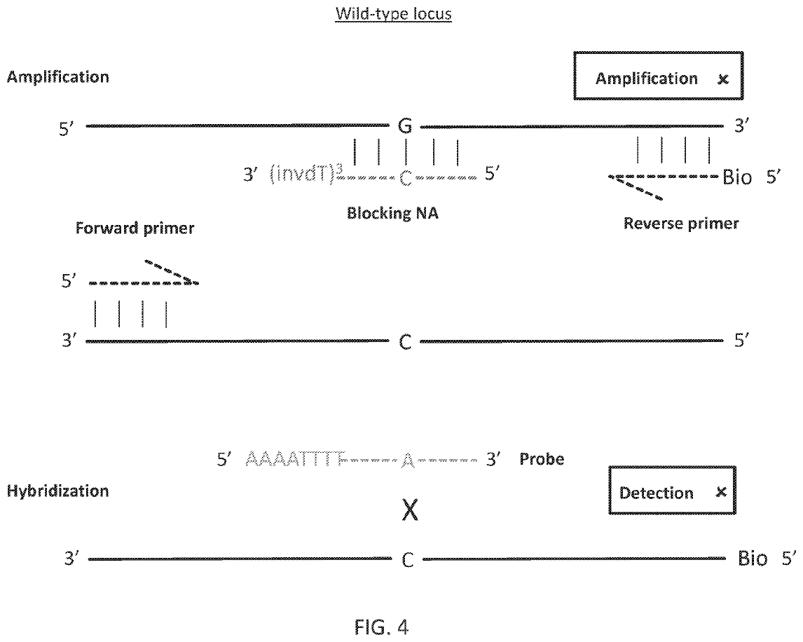

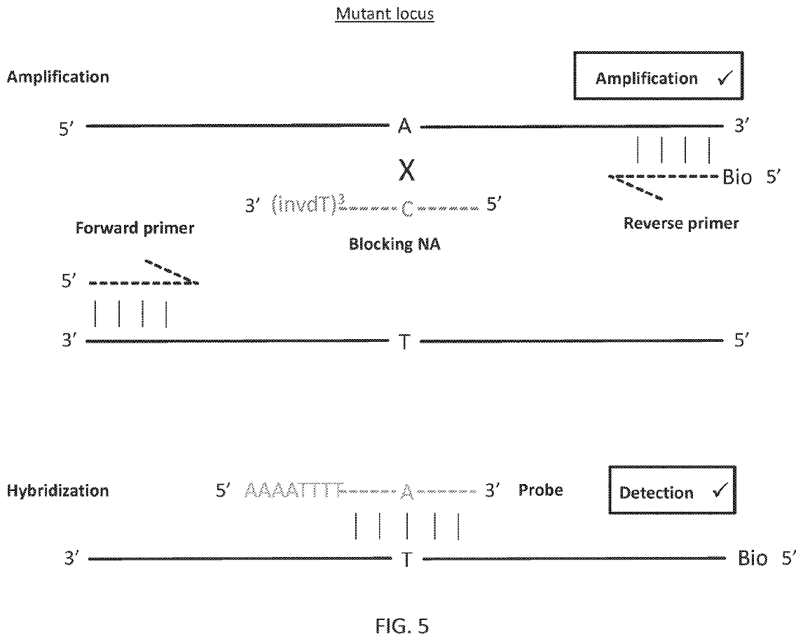

Certain aspects of the present disclosure relate to methods for detecting the presence of DNA mutations (e.g., one or more mutations in the KRAS, NRAS, PIK3CA, BRAF, EGFR, AKT1, MEK1, and/or HER2 genes) and/or RNA mutations (e.g., one or more mutations in the ALK, ROS, RET, NTRK1, and/or cMET genes) by using microcarriers, e.g., an encoded microcarrier described herein, or any of the microcarriers described in International Publication No. WO2016198954. The methods of the present disclosure employ encoded microcarrier(s) with some or all of the microcarrier features and aspects described herein, e.g., in sections IV, V, and VI. Advantageously, these encoded microcarriers allow for detection of DNA and/or RNA mutations in improved multiplex assays with a large number of potential unique microcarriers and reduced recognition error, as compared to traditional multiplex assays. A flowchart described an exemplary method for detection of mutations is provided in FIGS. 3 & 6, and exemplary PCR techniques are illustrated in FIGS. 4 & 5. The detection methods used herein may be performed in any suitable assay vessel known in the art, for example a microplate, petri dish, or any number of other well-known assay vessels.

In some embodiments, the methods of the present disclosure include isolating DNA and/or RNA from a sample. Standard molecular techniques known in the art allow for the isolation of DNA or RNA from a variety of different types of samples. DNA and RNA isolation kits suitable for a variety of samples are commercially available.

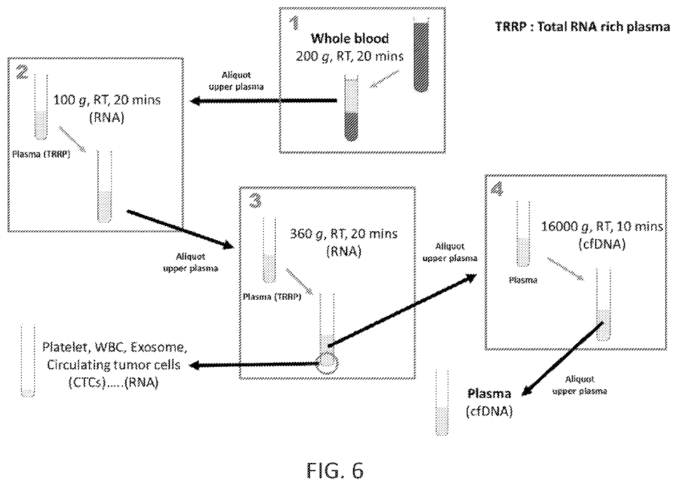

In some embodiments, the methods include isolating DNA and RNA from the same sample, e.g., a whole blood or plasma sample. An exemplary protocol for isolating DNA (e.g., circulating free or cell-free DNA, cfDNA) and RNA (e.g., from one or more of platelets, white blood cells, exosomes, circulating tumor cells, and free RNA) is shown in FIG. 6 (see also Best, M. G. et al. (2015) Cancer Cell 28:666-676). For example, in some embodiments, the methods include isolating total RNA-rich plasma (TRRP) by centrifuging a whole blood or plasma sample (e.g., by centrifuging whole blood at 200×g for 20 minutes and removing the TRRP), subjecting the TRRP to one or more centrifugation steps to generate an RNA fraction and a cell-free DNA (cfDNA) fraction (e.g., by centrifuging TRRP at 100×g for 20 minutes, removing the upper fraction, then centrifuging again for 360×g for 20 minutes), isolating DNA from the cfDNA fraction, and isolating RNA from the RNA fraction. An exemplary protocol is also provided in Example 2.

In some embodiments, the methods of the present disclosure use DNA from a sample at a concentration of between about 0.3 ng/μL to about 1 ng/μL. In some embodiments, the methods of the present disclosure use DNA from a sample at a concentration of at least about 0.3 ng/μL.

In some embodiments, the methods of the present disclosure use RNA from a sample at a concentration of between about 2 ng/μL to about 30 ng/L. In some embodiments, the methods of the present disclosure use RNA from a sample at a concentration of at least about 2 ng/μL.

In some embodiments, the methods of the present disclosure are used to detect, monitor, screen for, or monitor treatment response of lung cancer, e.g., in a patient from whom the sample is collected. As used herein, lung cancer can refer to various types of lung cancers, including without limitation non-small cell lung cancer (e.g., including subtypes such as adenocarcinoma, squamous cell carcinoma, and large cell carcinoma), small cell or oat cell cancer, and lung carcinoid tumors (e.g., bronchial carcinoids). A large body of research has implicated specific mutations in critical genes in many lung cancers. For example, mutations in KRAS, PIK3CA, BRAF, or EGFR are thought to be present in at least 40% of non-small-cell lung cancers (Rosell, R. et al. (2009) N. Engl J. Med. 361:958-967). Mutational screening is thought to improve patient outcomes, e.g., by identifying patients who are more likely to respond to targeted treatments, such as tyrosine kinase inhibitors.

The methods of the present disclosure can be used to detect analytes (e.g., DNA and/or RNA mutations) in any suitable solution. In some embodiments, the solution comprises a biological sample. In some embodiments, the solution comprises DNA or RNA isolated from a biological sample and, optionally, a buffer. Suitable buffers for DNA/RNA isolation are well-known in the art. Examples of biological samples include without limitation stool, blood, serum, plasma, urine, sputum, pleural effusion, bile, cerebrospinal fluid, interstitial fluid of skin or adipose tissue, saliva, tears, bronchial-alveolar lavage, oropharyngeal secretions, intestinal fluids, cervico-vaginal or uterine secretions, and seminal fluid. In some embodiments, the biological sample may be from a human. In other embodiments, the solution comprises a sample that is not a biological sample, such as an environmental sample, a sample prepared in a laboratory (e.g., a sample containing one or more analytes that have been prepared, isolated, purified, and/or synthesized), a fixed sample (e.g., a formalin-fixed, paraffin-embedded or FFPE sample), and so forth.

In some embodiments, the methods of the present disclosure include amplifying DNA (e.g., DNA isolated from a sample as described supra) by polymerase chain reaction (PCR). PCR techniques are well-known in the art. Briefly, a thermostable DNA polymerase is used to amplify copies of a DNA sequence of interest using template DNA strands (e.g., isolated from a sample and denatured) and a pair of oligonucleotide primers that are complementary to the 3′ ends of the sense and anti-sense strands (respectively) of the DNA template. The DNA polymerase is mixed in a reaction with both primers, all four deoxynucleotides (dNTPs), a buffer, magnesium ions (e.g., MgCl₂), and potassium ions (e.g., KCl), and optionally other ingredients. The reaction mixture is then subjected to multiple cycles (e.g., 20-40) of temperature changes that allow for denaturation of the DNA template, annealing of the primers to the denatured, single-stranded template, and primer extension by the DNA polymerase. Various DNA polymerases with different properties of interest (e.g., ability to amplify long or repetitive templates, high fidelity, hot start, etc.) have been characterized for use in PCR and are commercially available.

In some embodiments, the methods of the present disclosure include amplifying DNA from RNA (e.g., RNA isolated from a sample as described supra) by reverse transcription-polymerase chain reaction (RT-PCR). RT-PCR techniques are well-known in the art. Briefly, a reverse transcriptase and a primer complimentary to the 3′ end of an RNA molecule of interest are used for synthesizing first strand cDNA, which is then used as a template for PCR as described above using a DNA polymerase and a second primer for amplifying the strand opposite that amplified by the first primer. In some embodiments, the first primer comprises a 5′ label or modification, such as biotin. Various reverse transcriptases with different properties of interest (e.g., increased thermostability, modified RNase H activity, etc.) have been characterized for use in RT-PCR and are commercially available.

In some embodiments, the methods of the present disclosure include amplifying (e.g., by PCR or RT-PCR) from isolated DNA or RNA the loci of one or more mutations in one or more specific genes of interest. As herein, a “locus” of a DNA or RNA mutation comprises the mutation itself and sufficient adjacent sequence on one or both sides of the mutation for PCR amplification of, and/or probe hybridization to, the mutated DNA sequence (or, in the case of RNA, for PCR amplification of cDNA generated from the RNA). As is known in the art, the minimum sequence length sufficient for PCR amplification can be influenced by several factors, including without limitation the polymerase, the melting temperature of the primers, the propensity of the primers to form primer dimers, the ratio of the template to primers, etc. In some embodiments, the locus of a mutation comprises at least about 100 base pairs of adjacent sequence (i.e., including adjacent sequence both 5′ and 3′ to the mutation). In some embodiments, the locus of a mutation comprises less than or equal to about 200 base pairs of adjacent sequence (i.e., including adjacent sequence both 5′ and 3′ to the mutation). As described above, the locus of a DNA mutation can be amplified using a pair of primers specific to the locus, using the locus as the DNA or cDNA template.

In some embodiments, multiple PCR reactions can be used, e.g., to detect various DNA mutations. In some embodiments, each PCR reaction can include multiple primer pairs, each specific for a DNA mutation of interest.

Mutations

As used herein, amplifying the locus of a DNA or RNA mutation encompasses amplifying the mutant locus and/or the corresponding wild-type locus. It will be appreciated that in most instances, while many mutations can be screened in a multiplex assay, any individual sample will typically include one or very few of the mutations being screened. In some embodiments, a DNA mutation of the present disclosure refers to a mutation that is detected using DNA from a sample, and an RNA mutation of the present disclosure refers to a mutation that is detected using RNA from a sample (e.g., by generating cDNA and, subsequently, DNA from the RNA). It will be understood by one of skill in the art that mutations such as point mutations, deletions, insertions, and translocations/rearrangements may be present in both DNA and RNA from a sample (e.g., comprising tumor cells and/or non-tumor cells).

In some embodiments, the methods of the present disclosure include amplifying the loci of one or more mutations (e.g., DNA mutations) in a KRAS gene. KRAS encodes the KRAS proto-oncogene, a small GTPase frequently mutated in human cancers, also known as the Kirsten rag sarcoma viral oncogene homolog, PR310 c-K-ras oncogene, c-Ki-ras, c-Kirsten-ras, K-Ras2, K-ras p21, GTPase KRas, cellular c-Ki-ras2 proto-oncogene, cellular transforming proto-oncogene, oncogene KRAS2, transforming protein p21, and v-Ki-ras2 Kirsten rat sarcoma 2 viral oncogene homolog. In some embodiments, the KRAS gene is a human KRAS gene. In some embodiments, a human KRAS gene refers to the gene described by NCBI Entrez Gene ID No. 3845, including mutants and variants thereof. In other embodiments, the KRAS gene is from one of the following organisms: mouse (see, e.g., NCBI Entrez Gene ID No. 16653), rat (see, e.g., NCBI Entrez Gene ID No. 24525), cynomolgus monkey (see, e.g., NCBI Entrez Gene ID No. 102131483), fish (see, e.g., NCBI Entrez Gene ID No. 445289), dog (see, e.g., NCBI Entrez Gene ID No. 403871), cattle (see, e.g., NCBI Entrez Gene ID No. 541140), horse (see, e.g., NCBI Entrez Gene ID No. 100064473), chicken (see, e.g., NCBI Entrez Gene ID No. 418207), chimpanzee (see, e.g., NCBI Entrez Gene ID No. 473387), rhesus monkey (see, e.g., NCBI Entrez Gene ID No. 707977), or cat (see, e.g., NCBI Entrez Gene ID No. 751104).

A variety of KRAS mutations associated with cancer may be suitably detected by the methods described herein; see, e.g., Loyly, C., L. Horn, W. Pao. 2015. KRAS in Non-Small Cell Lung Cancer (NSCLC). My Cancer Genome at www.mycancergenome.org/content/disease/lung-cancer/kras/ (Updated June 18). Point mutations in KRASare found in 10-25% of lung cancers. KRAS mutations are seldom seen together with EGFR or ALK alterations in lung cancer and are more frequently observed in former or current smokers compared to never smokers. As per the COSMIC database (see cancer.sanger.ac.uk/cosmic), copy number gain in KRAS is observed in lung cancer (5.23%). Amplification of KRAS is also observed in certain cancers. KRAS mutations are generally associated with poor prognosis in NSCLC. However, a recent large retrospective study found no difference in prognosis by KRAS exon 12 mutation in patients with early stage NSCLC, calling into question the role of KRAS mutations a prognostic biomarker. Testing for KRAS mutations can be useful in determining a patient's sensitivity to tyrosine kinase inhibitors, such as MEK inhibitors. RAS mutations, such as in NSCLC, are associated with resistance to EGFR inhibitors, such as cetuximab, panitumumab, and erlotinib. FDA approved drugs sensitive to KRAS include sorafenib, regorafenib, palbociclib, cobimetinib, and trametinib. In some embodiments described herein, a KRAS mutation is named based on the resulting amino acid substitution/deletion/frameshift according to a human KRAS protein, e.g., as set forth in MTEYKLVVVGAGGVGKSALTIQLIQNHFVDEYDPTIEDSYRKQVVIDGETCLLDILDT AGQEEYSAMRDQYMRTGEGFLCVFAINNTKSFEDIHHYREQIKRVKDSEDVPMVLVG NKCDLPSRTVDTKQAQDLARSYGIPFIETSAKTRQGVDDAFYTLVREIRKHKEKMSKD GKKKKKKSKTKCVIM (SEQ ID NO:326). An exemplary human KRAS cDNA sequence is set forth in TGTGCTCGGAGCTCGATTTTCCTAGGCGGCGGCCGCGGCGGCGGAGGCAGCAGCG GCGGCGGCAGTGGCGGCGGCGAAGGTGGCGGCGGCTCGGCCAGTACTCCCGGCCC CCGCCATTTCGGACTGGGAGCGAGCGCGGCGCAGGCACTGAAGGCGGCGGCGGG GCCAGAGGCTCAGCGGCTCCCAGGCCTGCTGAAAATGACTGAATATAAACTTGTG GTAGTTGGAGCTGGTGGCGTAGGCAAGAGTGCCTTGACGATACAGCTAATTCAGA ATCATTTTGTGGACGAATATGATCCAACAATAGAGGATTCCTACAGGAAGCAAGT AGTAATTGATGGAGAAACCTGTCTCTTGGATATTCTCGACACAGCAGGTCAAGAG GAGTACAGTGCAATGAGGGACCAGTACATGAGGACTGGGGAGGGCTTTCTTTGTG TATTTGCCATAAATAATACTAAATCATTTGAAGATATTCACCATTATAGAGAACAA ATTAAAAGAGTTAAGGACTCTGAAGATGTACCTATGGTCCTAGTAGGAAATAAAT GTGATTTGCCTTCTAGAACAGTAGACACAAAACAGGCTCAGGACTTAGCAAGAAG TTATGGAATTCCTTTTATTGAAACATCAGCAAAGACAAGACAGAGAGTGGAGGAT GCTTTTTATACATTGGTGAGAGAGATCCGACAATACAGATTGAAAAAAATCAGCA AAGAAGAAAAGACTCCTGGCTGTGTGAAAATTAAAAAATGCATTATAATGTAATC TGGGTGTTGATGATGCCTTCTATACATTAGTTCGAGAAATTCGAAAACATAAAGAA AAGATGAGCAAAGATGGTAAAAAGAAGAAAAAG (SEQ ID NO:339). In some embodiments, a DNA mutation results in the mutation of G12 according to SEQ ID NO:326 or SEQ ID NO:339. For example, in some embodiments, a DNA mutation in aKRAS gene encodes or results in a G12C, G12D, or G12V mutated KRAS protein (numbering according to SEQ ID NO:326). These DNA mutations are also described by their nucleotide positions (rather than mutated polypeptide codons) in Table A1 infra. For example, in some embodiments, a DNA mutation in a KRAS gene results in a c.34G>C, c.34G>T, c.35G>A, mutation in the corresponding cDNA sequence of SEQ ID NO:339.

In some embodiments, a primer pair for amplifying the locus of a KRAS mutation (e.g., encoding or resulting in a G12C, G12D, or G12V mutated KRAS protein) comprises the sequences GTACTGGTGGAGTATTTGATAGTG (SEQ ID NO:1) and CGTCAAGGCACTCTTGCCTAC (SEQ ID NO:2), respectively.

In some embodiments, the methods of the present disclosure include amplifying the loci of one or more mutations (e.g., DNA mutations) in a BRAF gene. BRAF encodes the BRAF proto-oncogene, a serine/threonine kinase frequently mutated in human cancers, also known as B-Raf, BRAF1, B-RAF1, RAFB1, NS7, 94 kDa B-raf protein, p94, murine sarcoma viral (v-raf) oncogene homolog B1, v-raf murine sarcoma viral oncogene homolog B, and v-raf murine sarcoma viral oncogene homolog B1. In some embodiments, the BRAF gene is a human BRAF gene. In some embodiments, a human BRAF gene refers to the gene described by NCBI Entrez Gene ID No. 673, including mutants and variants thereof. In other embodiments, the BRAF gene is from one of the following organisms: mouse (see, e.g., NCBI Entrez Gene ID No. 109880), rat (see, e.g., NCBI Entrez Gene ID No. 114486), cynomolgus monkey (see, e.g., NCBI Entrez Gene ID No. 101866436), fish (see, e.g., NCBI Entrez Gene ID No. 403065), dog (see, e.g., NCBI Entrez Gene ID No. 475526), cattle (see, e.g., NCBI Entrez Gene ID No. 536051), horse (see, e.g., NCBI Entrez Gene ID No. 100065760), chicken (see, e.g., NCBI Entrez Gene ID No. 396239), chimpanzee (see, e.g., NCBI Entrez Gene ID No. 463781), rhesus monkey (see, e.g., NCBI Entrez Gene ID No. 693554), or cat (see, e.g., NCBI Entrez Gene ID No. 101092346).

A variety of BRAF mutations associated with cancer may be suitably detected by the methods described herein; see, e.g., Loyly, C., L. Horn, W. Pao. 2015. BRAF in Non-Small Cell Lung Cancer (NSCLC). My Cancer Genome at www.mycancergenome.org/content/disease/lung-cancer/braf/ (Updated June 18). The majority of the BRAF gain of function mutations alter residues in the kinase domain, most notably V600E, detectable by molecular testing. BRAF mutations and EGFR mutations are believed to be mutually exclusive. BRAF rearrangements, detectable by FISH, such as BRAF-KIAA1549, are also reported in some cancers. Amplifications are observed in certain cancers. Constitutive activation of BRAF has been observed in multiple cancers, including lung, where it leads to activation of the RAF/MEK/ERKpathway. Point mutations (1-4%) and copy number gain (1.43%) in BRAF are found in NSCLC. Prognosis associated with BRAFfusions is neutral in NSCLC when treated with chemotherapy. BRAF and MEK1/2 inhibitors are approved or under clinical evaluation as single agents or in combination for the treatment of BRAF mutant cancers. Some patients with V600E mutations have increased sensitivity to the BRAF inhibitors vemurafenib and dabrafinib. BRAF inhibition may ultimately result in resistance to BRAF or MEK inhibitors. Additionally, BRAF V600E mutations are resistant to EGFR therapies, such as cetuximab or panitumumab, as well as imatinib and sunitinib. While specific mutations and fusions, such as BRAF D594A/V and K483M, are insensitive to RAF inhibitors they are sensitive to MEK inhibitors. BRAF fusions, like BRAF-KIAA1549, are resistant to first generation BRAF inhibitors, such as vemurafenib, but second generation BRAF inhibitors are being investigated. FDA approved drugs sensitive to BRAF include dabrafenib, vemurafenib, cobimetinib, and trametinib. Targeting the RAF/MEK/ERK pathway may be efficacious in BRAF mutant cancers. In some embodiments described herein, a BRAF mutation is named based on the resulting amino acid substitution/deletion/frameshift according to a human BRAF protein, e.g., as set forth in MAALSGGGGGGAEPGQALFNGDMEPEAGAGAGAAASSAADPAIPEEVWNIKQMIKL TQEHIEALLDKFGGEHNPPSIYLEAYEEYTSKLDALQQREQQLLESLGNGTDFSVSSSA SMDTVTSSSSSSLSVLPSSLSVFQNPTDVARSNPKSPQKPIVRVFLPNKQRTVVPARCG VTVRDSLKKALMMRGLIPECCAVYRIQDGEKKPIGWDTDISWLTGEELHVEVLENVPL TTHNFVRKTFFTLAFCDFCRKLLFQGFRCQTCGYKFHQRCSTEVPLMCVNYDQLDLLF VSKFFEHHPIPQEEASLAETALTSGSSPSAPASDSIGPQILTSPSPSKSIPIPQPFRPADEDH RNQFGQRDRSSSAPNVHINTIEPVNIDDLIRDQGFRGDGGSTTGLSATPPASLPGSLTNV KALQKSPGPQRERKSSSSSEDRNRMKTLGRRDSSDDWEIPDGQITVGQRIGSGSFGTVY KGKWHGDVAVKMLNVTAPTPQQLQAFKNEVGVLRKTRHVNILLFMGYSTKPQLAIV TQWCEGSSLYHHLHIIETKFEMIKLIDIARQTAQGMDYLHAKSIIHRDLKSNNIFLHEDL TVKIGDFGLATVKSRWSGSHQFEQLSGSILWMAPEVIRMQDKNPYSFQSDVYAFGIVL YELMTGQLPYSNINNRDQIIFMVGRGYLSPDLSKVRSNCPKAMKRLMAECLKKKRDE RPLFPQILASIELLARSLPKIHRSASEPSLNRAGFQTEDFSLYACASPKTPIQAGGYGAFP VH (SEQ ID NO:329). An exemplary human BRAF cDNA sequence is set forth in ATGGCGGCGCTGAGCGGTGGCGGTGGTGGCGGCGCGGAGCCGGGCCAGGCTCTGT TCAACGGGGACATGGAGCCCGAGGCCGGCGCCGGCGCCGGCGCCGCGGCCTCTTC GGCTGCGGACCCTGCCATTCCGGAGGAGGTGTGGAATATCAAACAAATGATTAAG TTGACACAGGAACATATAGAGGCCCTATTGGACAAATTTGGTGGGGAGCATAATC CACCATCAATATATCTGGAGGCCTATGAAGAATACACCAGCAAGCTAGATGCACT CCAACAAAGAGAACAACAGTTATTGGAATCTCTGGGGAACGGAACTGATTTTTCT GTTTCTAGCTCTGCATCAATGGATACCGTTACATCTTCTTCCTCTTCTAGCCTTTCA GTGCTACCTTCATCTCTTTCAGTTTTTCAAAATCCCACAGATGTGGCACGGAGCAA CCCCAAGTCACCACAAAAACCTATCGTTAGAGTCTTCCTGCCCAACAAACAGAGG ACAGTGGTACCTGCAAGGTGTGGAGTTACAGTCCGAGACAGTCTAAAGAAAGCAC TGATGATGAGAGGTCTAATCCCAGAGTGCTGTGCTGTTTACAGAATTCAGGATGGA GAGAAGAAACCAATTGGTTGGGACACTGATATTTCCTGGCTTACTGGAGAAGAAT TGCATGTGGAAGTGTTGGAGAATGTTCCACTTACAACACACAACTTTGTACGAAAA ACGTTTTTCACCTTAGCATTTTGTGACTTTTGTCGAAAGCTGCTTTTCCAGGGTTTC CGCTGTCAAACATGTGGTTATAAATTTCACCAGCGTTGTAGTACAGAAGTTCCACT GATGTGTGTTAATTATGACCAACTTGATTTGCTGTTTGTCTCCAAGTTCTTTGAACA CCACCCAATACCACAGGAAGAGGCGTCCTTAGCAGAGACTGCCCTAACATCTGGA TCATCCCCTTCCGCACCCGCCTCGGACTCTATTGGGCCCCAAATTCTCACCAGTCC GTCTCCTTCAAAATCCATTCCAATTCCACAGCCCTTCCGACCAGCAGATGAAGATC ATCGAAATCAATTTGGGCAACGAGACCGATCCTCATCAGCTCCCAATGTGCATATA AACACAATAGAACCTGTCAATATTGATGACTTGATTAGAGACCAAGGATTTCGTG GTGATGGAGGATCAACCACAGGTTTGTCTGCTACCCCCCCTGCCTCATTACCTGGC TCACTAACTAACGTGAAAGCCTTACAGAAATCTCCAGGACCTCAGCGAGAAAGGA AGTCATCTTCATCCTCAGAAGACAGGAATCGAATGAAAACACTTGGTAGACGGGA CTCGAGTGATGATTGGGAGATTCCTGATGGGCAGATTACAGTGGGACAAAGAATT GGATCTGGATCATTTGGAACAGTCTACAAGGGAAAGTGGCATGGTGATGTGGCAG TGAAAATGTTGAATGTGACAGCACCTACACCTCAGCAGTTACAAGCCTTCAAAAA TGAAGTAGGAGTACTCAGGAAAACACGACATGTGAATATCCTACTCTTCATGGGC TATTCCACAAAGCCACAACTGGCTATTGTTACCCAGTGGTGTGAGGGCTCCAGCTT GTATCACCATCTCCATATCATTGAGACCAAATTTGAGATGATCAAACTTATAGATA TTGCACGACAGACTGCACAGGGCATGGATTACTTACACGCCAAGTCAATCATCCA CAGAGACCTCAAGAGTAATAATATATTTCTTCATGAAGACCTCACAGTAAAAATA GGTGATTTTGGTCTAGCTACAGTGAAATCTCGATGGAGTGGGTCCCATCAGTTTGA ACAGTTGTCTGGATCCATTTTGTGGATGGCACCAGAAGTCATCAGAATGCAAGATA AAAATCCATACAGCTTTCAGTCAGATGTATATGCATTTGGAATTGTTCTGTATGAA TTGATGACTGGACAGTTACCTTATTCAAACATCAACAACAGGGACCAGATAATTTT TATGGTGGGACGAGGATACCTGTCTCCAGATCTCAGTAAGGTACGGAGTAACTGT CCAAAAGCCATGAAGAGATTAATGGCAGAGTGCCTCAAAAAGAAAAGAGATGAG AGACCACTCTTTCCCCAAATTCTCGCCTCTATTGAGCTGCTGGCCCGCTCATTGCCA AAAATTCACCGCAGTGCATCAGAACCCTCCTTGAATCGGGCTGGTTTCCAAACAGA GGATTTTAGTCTATATGCTTGTGCTTCTCCAAAAACACCCATCCAGGCAGGGGGAT ATGGTGCGTTTCCTGTCCACTGA (SEQ ID NO:342). In some embodiments, a DNA mutation results in the mutation of V600 according to SEQ ID NO:329 or SEQ ID NO:342. For example, in some embodiments, a DNA mutation in a BRAF gene encodes or results in a V600E mutated BRAF protein (numbering according to SEQ ID NO:329). These DNA mutations are also described by their nucleotide positions (rather than mutated polypeptide codons) in Table A1 infra. It is to be appreciated that some references to the V600E BRAF mutation refer to it as V599E due to an early, incorrect BRAF protein sequence that was missing a codon at approximately amino acid 31 (see Garnett, M. J. and Marais, R. (2004) Cancer Cell 6:313-9 for description). For example, in some embodiments, a DNA mutation in a BRAF gene results in a c.1799T>A mutation in the corresponding cDNA sequence of SEQ ID NO:342.

In some embodiments, a primer pair for amplifying the locus of a BRAF mutation (e.g. encoding or resulting in a V600E mutated BRAF protein) comprises the sequences ATAGCCTCAATTCTTACCATCCACAAAATG (SEQ ID NO:9) and CAGATATATTTCTTCATGAAGACCTCACAGTAA (SEQ ID NO:10), respectively.

In some embodiments, the methods of the present disclosure include amplifying the loci of one or more mutations (e.g., DNA mutations) in an NRAS gene. NRAS encodes the NRAS proto-oncogene, a small GTPase frequently mutated in human cancers, also known as the Neuroblastoma RAS viral oncogene homolog, NCMS, NS6, ALPS4, CMNS, and NCMS. In some embodiments, the NRAS gene is a human NRAS gene. In some embodiments, a human NRAS gene refers to the gene described by NCBI Entrez Gene ID No. 4893, including mutants and variants thereof. In other embodiments, the NRAS gene is from one of the following organisms: mouse (see, e.g., NCBI Entrez Gene ID No. 18176), rat (see, e.g., NCBI Entrez Gene ID No. 24605), fish (see, e.g., NCBI Entrez Gene ID No. 30380), dog (see, e.g., NCBI Entrez Gene ID No. 403872), cattle (see, e.g., NCBI Entrez Gene ID No. 506322), horse (see, e.g., NCBI Entrez Gene ID No. 100059469), or chimpanzee (see, e.g., NCBI Entrez Gene ID No. 742713).

A variety of NRAS mutations associated with cancer are known and may be suitably detected by the methods described herein; see, e.g., Loyly, C., L. Horn, W. Pao. 2015. NRAS in Non-Small Cell Lung Cancer (NSCLC). My Cancer Genome at www.mycancergenome.org/content/disease/lung-cancer/nras/ (Updated June 18). The most frequent NRAS alterations observed in cancer are mutations at codons 12, 13, and 61 (90%), and within the phosphate binding loop/GI motif (residues 10-17), the switch II region (residues 59-67), and the G5 motif (residues 145-147). Somatic mutations in NRAS is rarely (0.2-1%) reported in primary NSCLC, but their role in carcinogenesis has been proven. Smoking and environmental carcinogens are associated with the etiology of NRAS mutated lung cancer. NRAS mutations have been correlated with metastases of NSCLC (1.5%). Somatic mutations in NRAS are generally associated with poor response to standard therapies. MEK inhibitors, such as selumetinib, are effective in treating cancer patients with RAS mutations. NRAS mutations, such as E63K0, are associated with resistance to anti-EGFR therapies, such as cetuximab and panitumumab, anti-BRAF therapies, such as vemurafenib and dabrafenib, ALK TKIs, and radiotherapy. In some embodiments described herein, an NRAS mutation is named based on the resulting amino acid substitution/deletion/frameshift according to a human NRAS protein, e.g., as set forth in MTEYKLVVVGAGGVGKSALTIQLIQNHFVDEYDPTIEDSYRKQVVIDGETCLLDILDT AGQEEYSAMRDQYMRTGEGFLCVFAINNSKSFADINLYREQIKRVKDSDDVPMVLVG NKCDLPTRTVDTKQAHELAKSYGIPFIETSAKTRQGVEDAFYTLVREIRQYRMKKLNS SDDGTQGCMGLPCVVM (SEQ ID NO:327). An exemplary human NRAS cDNA sequence is set forth in ATGACTGAGTACAAACTGGTGGTGGTTGGAGCAGGTGGTGTTGGGAAAAGCGCAC TGACAATCCAGCTAATCCAGAACCACTTTGTAGATGAATATGATCCCACCATAGAG GATTCTTACAGAAAACAAGTGGTTATAGATGGTGAAACCTGTTTGTTGGACATACT GGATACAGCTGGACAAGAAGAGTACAGTGCCATGAGAGACCAATACATGAGGAC AGGCGAAGGCTTCCTCTGTGTATTTGCCATCAATAATAGCAAGTCATTTGCGGATA TTAACCTCTACAGGGAGCAGATTAAGCGAGTAAAAGACTCGGATGATGTACCTAT GGTGCTAGTGGGAAACAAGTGTGATTTGCCAACAAGGACAGTTGATACAAAACAA GCCCACGAACTGGCCAAGAGTTACGGGATTCCATTCATTGAAACCTCAGCCAAGA CCAGACAGGGTGTTGAAGATGCTTTTTACACACTGGTAAGAGAAATACGCCAGTA CCGAATGAAAAAACTCAACAGCAGTGATGATGGGACTCAGGGTTGTATGGGATTG CCATGTGTGGTGATGTAA (SEQ ID NO:340). In some embodiments, a DNA mutation results in the mutation of Q61 according to SEQ ID NO:327 or SEQ ID NO:340. For example, in some embodiments, a DNA mutation in an NRAS gene encodes or results in a Q61L mutated NRAS protein (numbering according to SEQ ID NO:327). These DNA mutations are also described by their nucleotide positions (rather than mutated polypeptide codons) in Table A1 infra. For example, in some embodiments, a DNA mutation in an NRAS gene results in a c.182A>T mutation in the corresponding cDNA sequence of SEQ ID NO:340.

In some embodiments, a primer pair for amplifying the locus of an NRAS mutation (e.g., encoding or resulting in a Q61L mutated NRAS protein) comprises the sequences CCACACCCCCAGGATTCTT (SEQ ID NO:3) and TTGGTCTCTCATGGCACTGTACTC (SEQ ID NO:4), respectively.

In some embodiments, the methods of the present disclosure include amplifying the loci of one or more mutations (e.g., DNA mutations) in a PIK3CA gene. PIK3CA encodes the class I phosphatidylinositol-4,5-bisphosphate (PI) 3-kinase catalytic subunit, also known as the p110a protein, CLOVE, CWS5, MCM, MCAP, PI3K, CLAPO, MCMTC, and PI3K-alpha. In some embodiments, the PIK3CA gene is a human PIK3CA gene. In some embodiments, a human PIK3CA gene refers to the gene described by NCBI Entrez Gene ID No. 5290, including mutants and variants thereof. In other embodiments, the PIK3CA gene is from one of the following organisms: mouse (see, e.g., NCBI Entrez Gene ID No. 18706), rat (see, e.g., NCBI Entrez Gene ID No. 170911), fish (see, e.g., NCBI Entrez Gene ID No. 561737), dog (see, e.g., NCBI Entrez Gene ID No. 488084), cattle (see, e.g., NCBI Entrez Gene ID No. 282306), horse (see, e.g., NCBI Entrez Gene ID No. 100058141), chimpanzee (see, e.g., NCBI Entrez Gene ID No. 460858), or rhesus monkey (see, e.g., NCBI Entrez Gene ID No. 709959).

A variety of PIK3CA mutations associated with cancer are known and may be suitably detected by the methods described herein; see, e.g., Loyly, C., L. Horn, W. Pao. 2015. PIK3CA in Non-Small Cell Lung Cancer (NSCLC). My Cancer Genome at www.mycancergenome.org/content/disease/lung-cancer/pik3ca/ (Updated June 18). Activating mutations or amplification in PIK3CA result in constitutively active PI3K. Most PIK3CA gain of function mutations occur within the kinase (particularly residues 1043, 1047, and H1049R), alpha-helical (particularly residues E542K, E545K, and 546), and C-(particularly residues 345 and 420) domains. Other key domains that are less frequently mutated are the adaptor and linker domains. The PIK/AKT/mTOR pathway is dysregulated in 50-70% of NSCLC and PIK3CA mutations are detected in 1-5% of NSCLC. Copy number gain in PIK3CA is observed in lung cancer (16-20%), more frequently in sqNSCLC, and less frequently in SCLC (4.7%). PIK3CA is amplified in sqNSCLC (33-37%) and mutated (6.5-16%). PIK3CA activation is generally associated with poor prognosis. Tumors with constitutively active PIK3 have been proposed to be sensitive to agents targeting the PI3K/AKT/mTOR pathway. In some embodiments described herein, a PIK3CA mutation is named based on the resulting amino acid substitution/deletion/frameshift according to a human PIK3CA protein, e.g., as set forth in MPPRPSSGELWGIHLMPPRILVECLLPNGMIVTLECLREATLITIKHELFKEARKYPLHQ LLQDESSYIFVSVTQEAEREEFFDETRRLCDLRLFQPFLKVIEPVGNREEKILNREIGFAI GMPVCEFDMVKDPEVQDFRRNILNVCKEAVDLRDLNSPHSRAMYVYPPNVESSPELP KHIYNKLDKGQIIVVIWVIVSPNNDKQKYTLKINHDCVPEQVIAEAIRKKTRSMLLSSE QLKLCVLEYQGKYILKVCGCDEYFLEKYPLSQYKYIRSCIMLGRMPNLMLMAKESLY SQLPMDCFTMPSYSRRISTATPYMNGETSTKSLWVINSALRIKILCATYVNVNIRDIDKI YVRTGIYHGGEPLCDNVNTQRVPCSNPRWNEWLNYDIYIPDLPRAARLCLSICSVKGR KGAKEEHCPLAWGNINLFDYTDTLVSGKMALNLWPVPHGLEDLLNPIGVTGSNPNKE TPCLELEFDWFSSVVKFPDMSVIEEHANWSVSREAGFSYSHAGLSNRLARDNELREND KEQLKAISTRDPLSEITEQEKDFLWSHRHYCVTIPEILPKLLLSVKWNSRDEVAQMYCL VKDWPPIKPEQAMELLDCNYPDPMVRGFAVRCLEKYLTDDKLSQYLIQLVQVLKYEQ YLDNLLVRFLLKKALTNQRIGHFFFWHLKSEMHNKTVSQRFGLLLESYCRACGMYLK HLNRQVEAMEKLINLTDILKQEKKDETQKVQMKFLVEQMRRPDFMDALQGFLSPLNP AHQLGNLRLEECRIMSSAKRPLWLNWENPDIMSELLFQNNEIIFKNGDDLRQDMLTLQ IIRIMENIWQNQGLDLRMLPYGCLSIGDCVGLIEVVRNSHTIMQIQCKGGLKGALQFNS HTLHQWLKDKNKGEIYDAAIDLFTRSCAGYCVATFILGIGDRHNSNIMVKDDGQLFHI DFGHFLDHKKKKFGYKRERVPFVLTQDFLIVISKGAQECTKTREFERFQEMCYKAYLA IRQHANLFINLFSMMLGSGMPELQSFDDIAYIRKTLALDKTEQEALEYFMKQMNDAH HGGWTTKMDWIFHTIKQHALN (SEQ ID NO:328). An exemplary human PIK3CA cDNA sequence is set forth in ATGCCTCCACGACCATCATCAGGTGAACTGTGGGGCATCCACTTGATGCCCCCAAG AATCCTAGTAGAATGTTTACTACCAAATGGAATGATAGTGACTTTAGAATGCCTCC GTGAGGCTACATTAATAACCATAAAGCATGAACTATTTAAAGAAGCAAGAAAATA CCCCCTCCATCAACTTCTTCAAGATGAATCTTCTTACATTTTCGTAAGTGTTACTCA AGAAGCAGAAAGGGAAGAATTTTTTGATGAAACAAGACGACTTTGTGACCTTCGG CTTTTTCAACCCTTTTTAAAAGTAATTGAACCAGTAGGCAACCGTGAAGAAAAGAT CCTCAATCGAGAAATTGGTTTTGCTATCGGCATGCCAGTGTGTGAATTTGATATGG TTAAAGATCCAGAAGTACAGGACTTCCGAAGAAATATTCTGAACGTTTGTAAAGA AGCTGTGGATCTTAGGGACCTCAATTCACCTCATAGTAGAGCAATGTATGTCTATC CTCCAAATGTAGAATCTTCACCAGAATTGCCAAAGCACATATATAATAAATTAGAT AAAGGGCAAATAATAGTGGTGATCTGGGTAATAGTTTCTCCAAATAATGACAAGC AGAAGTATACTCTGAAAATCAACCATGACTGTGTACCAGAACAAGTAATTGCTGA AGCAATCAGGAAAAAAACTCGAAGTATGTTGCTATCCTCTGAACAACTAAAACTC TGTGTTTTAGAATATCAGGGCAAGTATATTTTAAAAGTGTGTGGATGTGATGAATA CTTCCTAGAAAAATATCCTCTGAGTCAGTATAAGTATATAAGAAGCTGTATAATGC TTGGGAGGATGCCCAATTTGATGTTGATGGCTAAAGAAAGCCTTTATTCTCAACTG CCAATGGACTGTTTTACAATGCCATCTTATTCCAGACGCATTTCCACAGCTACACC ATATATGAATGGAGAAACATCTACAAAATCCCTTTGGGTTATAAATAGTGCACTCA GAATAAAAATTCTTTGTGCAACCTACGTGAATGTAAATATTCGAGACATTGATAAG ATCTATGTTCGAACAGGTATCTACCATGGAGGAGAACCCTTATGTGACAATGTGAA CACTCAAAGAGTACCTTGTTCCAATCCCAGGTGGAATGAATGGCTGAATTATGATA TATACATTCCTGATCTTCCTCGTGCTGCTCGACTTTGCCTTTCCATTTGCTCTGTTAA AGGCCGAAAGGGTGCTAAAGAGGAACACTGTCCATTGGCATGGGGAAATATAAAC TTGTTTGATTACACAGACACTCTAGTATCTGGAAAAATGGCTTTGAATCTTTGGCC AGTACCTCATGGATTAGAAGATTTGCTGAACCCTATTGGTGTTACTGGATCAAATC CAAATAAAGAAACTCCATGCTTAGAGTTGGAGTTTGACTGGTTCAGCAGTGTGGTA AAGTTCCCAGATATGTCAGTGATTGAAGAGCATGCCAATTGGTCTGTATCCCGAGA AGCAGGATTTAGCTATTCCCACGCAGGACTGAGTAACAGACTAGCTAGAGACAAT GAATTAAGGGAAAATGACAAAGAACAGCTCAAAGCAATTTCTACACGAGATCCTC TCTCTGAAATCACTGAGCAGGAGAAAGATTTTCTATGGAGTCACAGACACTATTGT GTAACTATCCCCGAAATTCTACCCAAATTGCTTCTGTCTGTTAAATGGAATTCTAG AGATGAAGTAGCCCAGATGTATTGCTTGGTAAAAGATTGGCCTCCAATCAAACCT GAACAGGCTATGGAACTTCTGGACTGTAATTACCCAGATCCTATGGTTCGAGGTTT TGCTGTTCGGTGCTTGGAAAAATATTTAACAGATGACAAACTTTCTCAGTATTTAA TTCAGCTAGTACAGGTCCTAAAATATGAACAATATTTGGATAACTTGCTTGTGAGA TTTTTACTGAAGAAAGCATTGACTAATCAAAGGATTGGGCACTTTTTCTTTTGGCA TTTAAAATCTGAGATGCACAATAAAACAGTTAGCCAGAGGTTTGGCCTGCTTTTGG AGTCCTATTGTCGTGCATGTGGGATGTATTTGAAGCACCTGAATAGGCAAGTCGAG GCAATGGAAAAGCTCATTAACTTAACTGACATTCTCAAACAGGAGAAGAAGGATG AAACACAAAAGGTACAGATGAAGTTTTTAGTTGAGCAAATGAGGCGACCAGATTT CATGGATGCTCTACAGGGCTTTCTGTCTCCTCTAAACCCTGCTCATCAACTAGGAA ACCTCAGGCTTGAAGAGTGTCGAATTATGTCCTCTGCAAAAAGGCCACTGTGGTTG AATTGGGAGAACCCAGACATCATGTCAGAGTTACTGTTTCAGAACAATGAGATCA TCTTTAAAAATGGGGATGATTTACGGCAAGATATGCTAACACTTCAAATTATTCGT ATTATGGAAAATATCTGGCAAAATCAAGGTCTTGATCTTCGAATGTTACCTTATGG TTGTCTGTCAATCGGTGACTGTGTGGGACTTATTGAGGTGGTGCGAAATTCTCACA CTATTATGCAAATTCAGTGCAAAGGCGGCTTGAAAGGTGCACTGCAGTTCAACAG CCACACACTACATCAGTGGCTCAAAGACAAGAACAAAGGAGAAATATATGATGCA GCCATTGACCTGTTTACACGTTCATGTGCTGGATACTGTGTAGCTACCTTCATTTTG GGAATTGGAGATCGTCACAATAGTAACATCATGGTGAAAGACGATGGACAACTGT TTCATATAGATTTTGGACACTTTTTGGATCACAAGAAGAAAAAATTTGGTTATAAA CGAGAACGTGTGCCATTTGTTTTGACACAGGATTTCTTAATAGTGATTAGTAAAGG AGCCCAAGAATGCACAAAGACAAGAGAATTTGAGAGGTTTCAGGAGATGTGTTAC AAGGCTTATCTAGCTATTCGACAGCATGCCAATCTCTTCATAAATCTTTTCTCAATG ATGCTTGGCTCTGGAATGCCAGAACTACAATCTTTTGATGACATTGCATACATTCG AAAGACCCTAGCCTTAGATAAAACTGAGCAAGAGGCTTTGGAGTATTTCATGAAA CAAATGAATGATGCACATCATGGTGGCTGGACAACAAAAATGGATTGGATCTTCC ACACAATTAAACAGCATGCATTGAACTGA (SEQ ID NO:341). In some embodiments, a DNA mutation results in the mutation of E542, E545, or H1047 according to SEQ ID NO:328 or SEQ ID NO:341. For example, in some embodiments, a DNA mutation in a PIK3CA gene encodes or results in an E542K, E545K, or H1047R mutated PIK3CA protein (numbering according to SEQ ID NO:328). These DNA mutations are also described by their nucleotide positions (rather than mutated polypeptide codons) in Table A1 infra. For example, in some embodiments, a DNA mutation in a PIK3CA gene results in a c.1624G>A, c.1633G>A, or c.3140A>G mutation in the corresponding cDNA sequence of SEQ ID NO:341.

In some embodiments, a primer pair for amplifying the locus of a PIK3CA mutation (e.g., encoding or resulting in an E542K, or E545K mutated PIK3CA protein) comprises the sequences CAATTTCTACAAGAGATCCTCTCTCT (SEQ ID NO:5) and CTCCATTTTAGCACTTACCTGTGAC (SEQ ID NO:6), respectively. In some embodiments, a primer pair for amplifying the locus of a PIK3CA mutation (e.g., encoding or resulting in an H1047R mutated PIK3CA protein) comprises the sequences ACCCTAGCCTTAGATAAAACTGAGC (SEQ ID NO:7) and TTTGTTGTCCAGCCACCATGA (SEQ ID NO:8), respectively.

In some embodiments, the methods of the present disclosure include amplifying the loci of one or more mutations (e.g., DNA mutations) in an EGFR gene. EGFR encodes the epidermal growth factor receptor, a receptor tyrosine kinase frequently mutated in human cancers, also known as ERBB, ERBB1, HER1, NISBD2, PIG61, and mENA. In some embodiments, the EGFR gene is a human EGFR gene. In some embodiments, a human EGFR gene refers to the gene described by NCBI Entrez Gene ID No. 1956, including mutants and variants thereof. In other embodiments, the EGFR gene is from one of the following organisms: mouse (see, e.g., NCBI Entrez Gene ID No. 13649), rat (see, e.g., NCBI Entrez Gene ID No. 24329), dog (see, e.g., NCBI Entrez Gene ID No. 404306), cattle (see, e.g., NCBI Entrez Gene ID No. 407217), horse (see, e.g., NCBI Entrez Gene ID No. 100067755), chicken (see, e.g., NCBI Entrez Gene ID No. 396494), or cat (see, e.g., NCBI Entrez Gene ID No. 100510799).

A variety of EGFR mutations associated with cancer are known and may be suitably detected by the methods described herein; see, e.g., Loyly, C., L. Horn, W. Pao. 2015. EGFR in Non-Small Cell Lung Cancer (NSCLC). My Cancer Genome at www.mycancergenome.org/content/disease/lung-cancer/egfr/ (Updated June 18). EGFR alterations, including overexpression, amplification, and mutation, are involved in development of numerous solid tumors. The most frequent EGFR mutations in cancer are in the kinase domain, including indels between residues 739-757 and mutations of L858, leading to constitutive activation. Lung cancer point mutations in EGFR occur 28.94% of the time, while copy number gain is found in 5.06% of lung cancers. EGFR mutations in lung cancer are associated with adenocarcinoma in female nonsmokers of Asian ethnicity. Specific point mutations are frequently encountered in NSCLC: G719, T790M, C797S, and L861, and have distinct therapeutic relevance. Lung cancer patients with mutations in exons 18, 19, and 21 may be sensitive to EGFR inhibitors, such as erlotinib and gefitinib. Acquired mutations in exon 20, such as T790M, are known to be resistant to first generation EGFR TKIs. Later generation EGFR TKIs, such as afatinib and osimertinib, were developed to counter resistant variants, including T790M. Other mutations, such as C797S, L844V, and L718Q, may be responsible for resistance to third generation TKIs. EGFR alterations may also drive resistance to ALK-targeted therapy. FDA approved EGFR inhibitors include osimertinib, gefitinib, erlotinib, necitumumab, and afatinib. Osimertinib is approved for the treatment of T790M lung cancer. Gefitinib is approved for metastatic NSCLC with EGFR exon 19 deletions or exon 21 (L858R) substitution mutations as detected by an FDA-approved test. Other FDA approved drugs sensitive to EGFR include lapatinib, vandetanib, cetuximab, and panitumumab. In some embodiments described herein, an EGFR mutation is named based on the resulting amino acid substitution/deletion/frameshift according to a human EGFR protein, e.g., as set forth in

(SEQ ID NO: 330)
MRPSGTAGAALLALLAALCPASRALEEKKVCQGTSNKLTQLGTFEDHFLS
LQRMFNNCEVVLGNLEITYVQRNYDLSFLKTIQEVAGYVLIALNTVERIP
LENLQIIRGNMYYENSYALAVLSNYDANKTGLKELPMRNLQEILHGAVRF
SNNPALCNVESIQWRDIVSSDFLSNMSMDFQNHLGSCQKCDPSCPNGSCW
GAGEENCQKLTKIICAQQCSGRCRGKSPSDCCHNQCAAGCTGPRESDCLV
CRKFRDEATCKDTCPPLMLYNPTTYQMDVNPEGKYSFGATCVKKCPRNYV
VTDHGSCVRACGADSYEMEEDGVRKCKKCEGPCRKVCNGIGIGEFKDSLS
INATNIKHFKNCTSISGDLHILPVAFRGDSFTHTPPLDPQELDILKTVKE
ITGFLLIQAWPENRTDLHAFENLEIIRGRTKQHGQFSLAVVSLNITSLGL
RSLKEISDGDVIISGNKNLCYANTINWKKLFGTSGQKTKIISNRGENSCK
ATGQVCHALCSPEGCWGPEPRDCVSCRNVSRGRECVDKCNLLEGEPREFV
ENSECIQCHPECLPQAMNITCTGRGPDNCIQCAHYIDGPHCVKTCPAGVM
GENNTLVWKYADAGHVCHLCHPNCTYGCTGPGLEGCPTNGPKIPSIATGM
VGALLLLLVVALGIGLFMRRRHIVRKRTLRRLLQERELVEPLTPSGEAPN
QALLRILKETEFKKIKVLGSGAFGTVYKGLWIPEGEKVKIPVAIKELREA
TSPKANKEILDEAYVMASVDNPHVCRLLGICLTSTVQLITQLMPFGCLLD
YVREHKDNIGSQYLLNWCVQIAKGMNYLEDRRLVHRDLAARNVLVKTPQH
VKITDFGLAKLLGAEEKEYHAEGGKVPIKWMALESILHRIYTHQSDVWSY
GVTVWELMTFGSKPYDGIPASEISSILEKGERLPQPPICTIDVYMIMVKC
WMIDADSRPKFRELIIEFSKMARDPQRYLVIQGDERMHLPSPTDSNFYRA
LMDEEDMDDVVDADEYLIPQQGFFSSPSTSRTPLLSSLSATSNNSTVACI
DRNGLQSCPIKEDSFLQRYSSDPTGALTEDSIDDTFLPVPEYINQSVPKR
PAGSVQNPVYHNQPLNPAPSRDPHYQDPHSTAVGNPEYLNTVQPTCVNST
FDSPAHWAQKGSHQISLDNPDYQQDFFPKEAKPNGIFKGSTAENAEYLRV
APQSSEFIGA.

An exemplary human EGFR cDNA sequence is set forth in ATGCGACCCTCCGGGACGGCCGGGGCAGCGCTCCTGGCGCTGCTGGCTGCGCTCT GCCCGGCGAGTCGGGCTCTGGAGGAAAAGAAAGTTTGCCAAGGCACGAGTAACA AGCTCACGCAGTTGGGCACTTTTGAAGATCATTTTCTCAGCCTCCAGAGGATGTTC AATAACTGTGAGGTGGTCCTTGGGAATTTGGAAATTACCTATGTGCAGAGGAATTA TGATCTTTCCTTCTTAAAGACCATCCAGGAGGTGGCTGGTTATGTCCTCATTGCCCT CAACACAGTGGAGCGAATTCCTTTGGAAAACCTGCAGATCATCAGAGGAAATATG TACTACGAAAATTCCTATGCCTTAGCAGTCTTATCTAACTATGATGCAAATAAAAC CGGACTGAAGGAGCTGCCCATGAGAAATTTACAGGAAATCCTGCATGGCGCCGTG CGGTTCAGCAACAACCCTGCCCTGTGCAACGTGGAGAGCATCCAGTGGCGGGACA TAGTCAGCAGTGACTTTCTCAGCAACATGTCGATGGACTTCCAGAACCACCTGGGC AGCTGCCAAAAGTGTGATCCAAGCTGTCCCAATGGGAGCTGCTGGGGTGCAGGAG AGGAGAACTGCCAGAAACTGACCAAAATCATCTGTGCCCAGCAGTGCTCCGGGCG CTGCCGTGGCAAGTCCCCCAGTGACTGCTGCCACAACCAGTGTGCTGCAGGCTGCA CAGGCCCCCGGGAGAGCGACTGCCTGGTCTGCCGCAAATTCCGAGACGAAGCCAC GTGCAAGGACACCTGCCCCCCACTCATGCTCTACAACCCCACCACGTACCAGATGG ATGTGAACCCCGAGGGCAAATACAGCTTTGGTGCCACCTGCGTGAAGAAGTGTCC CCGTAATTATGTGGTGACAGATCACGGCTCGTGCGTCCGAGCCTGTGGGGCCGAC AGCTATGAGATGGAGGAAGACGGCGTCCGCAAGTGTAAGAAGTGCGAAGGGCCTT GCCGCAAAGTGTGTAACGGAATAGGTATTGGTGAATTTAAAGACTCACTCTCCATA AATGCTACGAATATTAAACACTTCAAAAACTGCACCTCCATCAGTGGCGATCTCCA CATCCTGCCGGTGGCATTTAGGGGTGACTCCTTCACACATACTCCTCCTCTGGATC CACAGGAACTGGATATTCTGAAAACCGTAAAGGAAATCACAGGGTTTTTGCTGAT TCAGGCTTGGCCTGAAAACAGGACGGACCTCCATGCCTTTGAGAACCTAGAAATC ATACGCGGCAGGACCAAGCAACATGGTCAGTTTTCTCTTGCAGTCGTCAGCCTGAA CATAACATCCTTGGGATTACGCTCCCTCAAGGAGATAAGTGATGGAGATGTGATA ATTTCAGGAAACAAAAATTTGTGCTATGCAAATACAATAAACTGGAAAAAACTGT TTGGGACCTCCGGTCAGAAAACCAAAATTATAAGCAACAGAGGTGAAAACAGCTG CAAGGCCACAGGCCAGGTCTGCCATGCCTTGTGCTCCCCCGAGGGCTGCTGGGGC CCGGAGCCCAGGGACTGCGTCTCTTGCCGGAATGTCAGCCGAGGCAGGGAATGCG TGGACAAGTGCAACCTTCTGGAGGGTGAGCCAAGGGAGTTTGTGGAGAACTCTGA GTGCATACAGTGCCACCCAGAGTGCCTGCCTCAGGCCATGAACATCACCTGCACA GGACGGGGACCAGACAACTGTATCCAGTGTGCCCACTACATTGACGGCCCCCACT GCGTCAAGACCTGCCCGGCAGGAGTCATGGGAGAAAACAACACCCTGGTCTGGAA GTACGCAGACGCCGGCCATGTGTGCCACCTGTGCCATCCAAACTGCACCTACGGAT GCACTGGGCCAGGTCTTGAAGGCTGTCCAACGAATGGGCCTAAGATCCCGTCCAT CGCCACTGGGATGGTGGGGGCCCTCCTCTTGCTGCTGGTGGTGGCCCTGGGGATCG GCCTCTTCATGCGAAGGCGCCACATCGTTCGGAAGCGCACGCTGCGGAGGCTGCT GCAGGAGAGGGAGCTTGTGGAGCCTCTTACACCCAGTGGAGAAGCTCCCAACCAA GCTCTCTTGAGGATCTTGAAGGAAACTGAATTCAAAAAGATCAAAGTGCTGGGCT CCGGTGCGTTCGGCACGGTGTATAAGGGACTCTGGATCCCAGAAGGTGAGAAAGT TAAAATTCCCGTCGCTATCAAGGAATTAAGAGAAGCAACATCTCCGAAAGCCAAC AAGGAAATCCTCGATGAAGCCTACGTGATGGCCAGCGTGGACAACCCCCACGTGT GCCGCCTGCTGGGCATCTGCCTCACCTCCACCGTGCAGCTCATCACGCAGCTCATG CCCTTCGGCTGCCTCCTGGACTATGTCCGGGAACACAAAGACAATATTGGCTCCCA GTACCTGCTCAACTGGTGTGTGCAGATCGCAAAGGGCATGAACTACTTGGAGGAC CGTCGCTTGGTGCACCGCGACCTGGCAGCCAGGAACGTACTGGTGAAAACACCGC AGCATGTCAAGATCACAGATTTTGGGCTGGCCAAACTGCTGGGTGCGGAAGAGAA AGAATACCATGCAGAAGGAGGCAAAGTGCCTATCAAGTGGATGGCATTGGAATCA ATTTTACACAGAATCTATACCCACCAGAGTGATGTCTGGAGCTACGGGGTGACTGT TTGGGAGTTGATGACCTTTGGATCCAAGCCATATGACGGAATCCCTGCCAGCGAG ATCTCCTCCATCCTGGAGAAAGGAGAACGCCTCCCTCAGCCACCCATATGTACCAT CGATGTCTACATGATCATGGTCAAGTGCTGGATGATAGACGCAGATAGTCGCCCA AAGTTCCGTGAGTTGATCATCGAATTCTCCAAAATGGCCCGAGACCCCCAGCGCTA CCTTGTCATTCAGGGGGATGAAAGAATGCATTTGCCAAGTCCTACAGACTCCAACT TCTACCGTGCCCTGATGGATGAAGAAGACATGGACGACGTGGTGGATGCCGACGA GTACCTCATCCCACAGCAGGGCTTCTTCAGCAGCCCCTCCACGTCACGGACTCCCC TCCTGAGCTCTCTGAGTGCAACCAGCAACAATTCCACCGTGGCTTGCATTGATAGA AATGGGCTGCAAAGCTGTCCCATCAAGGAAGACAGCTTCTTGCAGCGATACAGCT CAGACCCCACAGGCGCCTTGACTGAGGACAGCATAGACGACACCTTCCTCCCAGT GCCTGAATACATAAACCAGTCCGTTCCCAAAAGGCCCGCTGGCTCTGTGCAGAATC CTGTCTATCACAATCAGCCTCTGAACCCCGCGCCCAGCAGAGACCCACACTACCAG GACCCCCACAGCACTGCAGTGGGCAACCCCGAGTATCTCAACACTGTCCAGCCCA CCTGTGTCAACAGCACATTCGACAGCCCTGCCCACTGGGCCCAGAAAGGCAGCCA CCAAATTAGCCTGGACAACCCTGACTACCAGCAGGACTTCTTTCCCAAGGAAGCC AAGCCAAATGGCATCTTTAAGGGCTCCACAGCTGAAAATGCAGAATACCTAAGGG TCGCGCCACAAAGCAGTGAATTTATTGGAGCATGA (SEQ ID NO:343). In some embodiments, a DNA mutation results in the mutation of G719, E746, T790M, C797S, S768I, V769, H773, D770, or L858 according to SEQ ID NO:330 or SEQ ID NO:343. For example, in some embodiments, a DNA mutation in an EGFR gene encodes or results in a G719A, E746_A750del, T790M, C797S, S768I, V769_D770insASV, H773_V774insH. D770_N771insG, D770_N771insSVD, or L858R mutated EGFR protein (numbering according to SEQ ID NO:330). These DNA mutations are also described by their nucleotide positions (rather than mutated polypeptide codons) in Table A1 infra. For example, in some embodiments, a DNA mutation in an EGFR gene results in a c.2156G>C, c.2235_2249del15, c.2236_2250del15, c.2369C>T, c.2389T>A, c.2390G>C, c.2303G>T, c.2307_2308ins9GCCAGCGTG, c.2319_2320insCAC, c.2310_2311insGGT, c.2311_2312ins9GCGTGGACA, c.2309_2310AC>CCAGCGTGGAT, or c.2573T>G mutation in the corresponding cDNA sequence of SEQ ID NO:343.

In some embodiments, a primer pair for amplifying the locus of an EGFR mutation (e.g., encoding or resulting in a G719A mutated EGFR protein) comprises the sequences CTTGTGGAGCCTCTTACACCC (SEQ ID NO:11) and TGCCGAACGCACCGGA (SEQ ID NO:12), respectively. In some embodiments, a primer pair for amplifying the locus of an EGFR mutation (e.g., encoding or resulting in an E746_A750del mutated EGFR protein) comprises the sequences GCCAGTTAACGTCTTCCTTCTC (SEQ ID NO:13) and ATCGAGGATTTCCTTGTTGGCTT (SEQ ID NO:14), respectively. In some embodiments, a primer pair for amplifying the locus of an EGFR mutation (e.g., encoding or resulting in a T790M, C797S, S768I, V769_D770insASV, H773_V774insH, D770_N77linsG, D770_N771insSVD, or V769_D770insASV mutated EGFR protein) comprises the sequences CCTCCACCGTGCAGATCATC (SEQ ID NO:15) and TTCCCTGATTACCTTTGCGAT (SEQ ID NO:16), respectively. In some embodiments, a primer pair for amplifying the locus of an EGFR mutation (e.g., encoding or resulting in a T790M, C797S, S768I, V769_D770insASV, H773_V774insH, D770_N771insG, D770_N771insSVD, or V769_D770insASV mutated EGFR protein) comprises the sequences CCTCCACCGTGCAGATCATC (SEQ ID NO:15) and TTCCCTGATTACCTTTGCGAT (SEQ ID NO:16), respectively. In some embodiments, a primer pair for amplifying the locus of an EGFR mutation (e.g., encoding or resulting in an S768 mutated EGFR protein) comprises the sequences CCACACTGACGTGCCTCT (SEQ ID NO:511) and GCACACGTAGGGGTTGTCCAAGA (SEQ ID NO:512), respectively. In some embodiments, a primer pair for amplifying the locus of an EGFR mutation (e.g., encoding or resulting in a V769_D770insASV mutated EGFR protein) comprises the sequences CCACACTGACGTGCCTCT (SEQ ID NO:513) and GTACACGCTGGCCACGCCG (SEQ ID NO:514), respectively. In some embodiments, a primer pair for amplifying the locus of an EGFR mutation (e.g., encoding or resulting in an H773_V774insH mutated EGFR protein) comprises the sequences CCACACTGACGTGCCTCT (SEQ ID NO:515) and CAGGCGGCACACGTGAT (SEQ ID NO:516), respectively. In some embodiments, a primer pair for amplifying the locus of an EGFR mutation (e.g., encoding or resulting in a D770_N771insG mutated EGFR protein) comprises the sequences CCACACTGACGTGCCTCT (SEQ ID NO:517) and AGGCGGCACACGTGCGGGTTAC (SEQ ID NO:518), respectively. In some embodiments, a primer pair for amplifying the locus of an EGFR mutation (e.g., encoding or resulting in an L858R mutated EGFR protein) comprises the sequences GGAGGACCGTCGCTTGG (SEQ ID NO: 17).

In some embodiments, the methods of the present disclosure include amplifying the loci of one or more mutations (e.g., DNA mutations) in an AKT1 gene. AKT1 encodes the RAC-alpha serine/threonine protein kinase frequently mutated in human cancers, also known as AKT, CWS6, PKB, PKB-ALPHA, PRKBA, RAC, and RAC-ALPHA. In some embodiments, the AKT1 gene is a human AKT1 gene. In some embodiments, a human AKT1 gene refers to the gene described by NCBI Entrez Gene ID No. 207, including mutants and variants thereof. In other embodiments, the AKT1 gene is from one of the following organisms: mouse (see, e.g., NCBI Entrez Gene ID No. 11651), rat (see, e.g., NCBI Entrez Gene ID No. 24185), fish (see, e.g., NCBI Entrez Gene ID No. 101910198), dog (see, e.g., NCBI Entrez Gene ID No. 490878), cattle (see, e.g., NCBI Entrez Gene ID No. 280991), chicken (see, e.g., NCBI Entrez Gene ID No. 395928), or chimpanzee (see, e.g., NCBI Entrez Gene ID No. 740898).

A variety of AKT mutations associated with cancer are known and may be suitably detected by the methods described herein; see, e.g., Loyly, C., L. Horn, W. Pao. 2015. AKT1 in Non-Small Cell Lung Cancer (NSCLC). My Cancer Genome at www.mycancergenome.org/content/disease/lung-cancer/akt1/ (Updated June 18). The AKT1 proto-oncogene, on chromosome 14, encodes a serine-threonine protein kinase (PKB) and a downstream effector of PI3K that plays a role in cell proliferation, survival, apoptosis, cell growth, glucose metabolism, genome stability, transcription, and neovascularization. AKT1 promotes constitutive activation of the mTOR signaling pathway and the glycolytic phenotype in multiple cancers. The most frequent AKT1 alteration observed in cancer is E17K in the pleckstrin homology domain. Amplification and overexpression of AKT1 have also been observed in certain cancers. Point mutations in AKT1 occur in lung cancer (0.6%), but more frequently in sqNSCLC (2-5%). In lung cancer, 1.01% have copy number gain in AKT1. Testing for AKT1 mutations can be useful for determining sensitivity to various drugs, such as PI3K/AKT/mTOR inhibitors, including everolimus. Constitutive activation of AKT1 is associated with resistance to chemotherapy or radiation therapy in a variety of cancers, including EGFR-TKIs in lung cancer. While no direct AKT inhibitor has been yet approved for cancer, FDA approved drugs sensitive to AKT1 include everolimus and temsirolimus. Preclinical data report inhibition of certain AKT1 mutations, including E17K, by AKT inhibitors. Various allosteric and ATP-competitive AKT inhibitors are currently in clinical trials. In some embodiments described herein, an AKT mutation is named based on the resulting amino acid substitution/deletion/frameshift according to a human AKT1 protein, e.g., as set forth in MSDVAIVKEGWLHKRGEYIKTWRPRYFLLKNDGTFIGYKERPQDVDQREAPLNNFSVA QCQLMKTERPRPNTFIIRCLQWTTVIERTFHVETPEEREEWTTAIQTVADGLKKQEEEEM DFRSGSPSDNSGAEEMEVSLAKPKHRVTMNEFEYLKLLGKGTFGKVILVKEKATGRYY AMKILKKEVIVAKDEVAHTLTENRVLQNSRHPFLTALKYSFQTHDRLCFVMEYANGGE LFFHLSRERVFSEDRARFYGAEIVSALDYLHSEKNVVYRDLKLENLMLDKDGHIKITDF GLCKEGIKDGATMKTFCGTPEYLAPEVLEDNDYGRAVDWWGLGVVMYEMMCGRLPF YNQDHEKLFELILMEEIRFPRTLGPEAKSLLSGLLKKDPKQRLGGGSEDAKEIMQHRFFA GIVWQHVYEKKLSPPFKPQVTSETDTRYFDEEFTAQMITITPPDQDDSMECVDSERRPHF PQFSYSASGTA (SEQ ID NO:331). An exemplary human AKT1 cDNA sequence is set forth in ATGAGCGACGTGGCTATTGTGAAGGAGGGTTGGCTGCACAAACGAGGGGAGTACAT CAAGACCTGGCGGCCACGCTACTTCCTCCTCAAGAATGATGGCACCTTCATTGGCTA CAAGGAGCGGCCGCAGGATGTGGACCAACGTGAGGCTCCCCTCAACAACTTCTCTG TGGCGCAGTGCCAGCTGATGAAGACGGAGCGGCCCCGGCCCAACACCTTCATCATC CGCTGCCTGCAGTGGACCACTGTCATCGAACGCACCTTCCATGTGGAGACTCCTGAG GAGCGGGAGGAGTGGACAACCGCCATCCAGACTGTGGCTGACGGCCTCAAGAAGCA GGAGGAGGAGGAGATGGACTTCCGGTCGGGCTCACCCAGTGACAACTCAGGGGCTG AAGAGATGGAGGTGTCCCTGGCCAAGCCCAAGCACCGCGTGACCATGAACGAGTTT GAGTACCTGAAGCTGCTGGGCAAGGGCACTTTCGGCAAGGTGATCCTGGTGAAGGA GAAGGCCACAGGCCGCTACTACGCCATGAAGATCCTCAAGAAGGAAGTCATCGTGG CCAAGGACGAGGTGGCCCACACACTCACCGAGAACCGCGTCCTGCAGAACTCCAGG CACCCCTTCCTCACAGCCCTGAAGTACTCTTTCCAGACCCACGACCGCCTCTGCTTTG TCATGGAGTACGCCAACGGGGGCGAGCTGTTCTTCCACCTGTCCCGGGAGCGTGTGT TCTCCGAGGACCGGGCCCGCTTCTATGGCGCTGAGATTGTGTCAGCCCTGGACTACC TGCACTCGGAGAAGAACGTGGTGTACCGGGACCTCAAGCTGGAGAACCTCATGCTG GACAAGGACGGGCACATTAAGATCACAGACTTCGGGCTGTGCAAGGAGGGGATCAA GGACGGTGCCACCATGAAGACCTTTTGCGGCACACCTGAGTACCTGGCCCCCGAGG TGCTGGAGGACAATGACTACGGCCGTGCAGTGGACTGGTGGGGGCTGGGCGTGGTC ATGTACGAGATGATGTGCGGTCGCCTGCCCTTCTACAACCAGGACCATGAGAAGCTT TTTGAGCTCATCCTCATGGAGGAGATCCGCTTCCCGCGCACGCTTGGTCCCGAGGCC AAGTCCTTGCTTTCAGGGCTGCTCAAGAAGGACCCCAAGCAGAGGCTTGGCGGGGG CTCCGAGGACGCCAAGGAGATCATGCAGCATCGCTTCTTTGCCGGTATCGTGTGGCA GCACGTGTACGAGAAGAAGCTCAGCCCACCCTTCAAGCCCCAGGTCACGTCGGAGA CTGACACCAGGTATTTTGATGAGGAGTTCACGGCCCAGATGATCACCATCACACCAC CTGACCAAGATGACAGCATGGAGTGTGTGGACAGCGAGCGCAGGCCCCACTTCCCC CAGTTCTCCTACTCGGCCAGCGGCACGGCCTGA (SEQ ID NO:344). In some embodiments, a DNA mutation results in the mutation of E17 according to SEQ ID NO:331 or SEQ ID NO:344. For example, in some embodiments, a DNA mutation in an AKT1 gene encodes or results in an E17K mutated AKT1 protein (numbering according to SEQ ID NO:331). This DNA mutation is also described by its nucleotide positions (rather than mutated polypeptide codons) in Table A1 infra. For example, in some embodiments, a DNA mutation in an AKT1 gene results in a c.49G>A mutation in the corresponding cDNA sequence of SEQ ID NO:344.

In some embodiments, a primer pair for amplifying the locus of an AKT1 mutation (e.g., encoding or resulting in an E17K mutated AKT1 protein) comprises the sequences GAGGGTCTGACGGGTAGAGTG (SEQ ID NO:380) and TGGCCGCCAGGTCTTGATGTA (SEQ ID NO:381), respectively.

In some embodiments, the methods of the present disclosure include amplifying the loci of one or more mutations (e.g., DNA mutations) in a MEK1 gene. MEK1 encodes the dual specificity mitogen-activated protein kinase kinase 1 frequently mutated in human cancers, also known as MAP2K1, CFC3, MAPKK1, MKK1, and PRKMK1. In some embodiments, the MEK1 gene is a human MEK1 gene. In some embodiments, a human MEK1 gene refers to the gene described by NCBI Entrez Gene ID No. 5604, including mutants and variants thereof. In other embodiments, the MEK1 gene is from one of the following organisms: mouse (see, e.g., NCBI Entrez Gene ID No. 26395), rat (see, e.g., NCBI Entrez Gene ID No. 170851), fish (see, e.g., NCBI Entrez Gene ID No. 406728), dog (see, e.g., NCBI Entrez Gene ID No. 478347), cattle (see, e.g., NCBI Entrez Gene ID No. 533199), horse (see, e.g., NCBI Entrez Gene ID No. 100065996), chimpanzee (see, e.g., NCBI Entrez Gene ID No. 450188), or rhesus monkey (see, e.g., NCBI Entrez Gene ID No. 710415).

A variety of MEK1 mutations associated with cancer are known and may be suitably detected by the methods described herein; see, e.g., Loyly, C., L. Horn, W. Pao. 2015. MEK1 (MAP2K1) in Non-Small Cell Lung Cancer (NSCLC). My Cancer Genome at www.mycancergenome.org/content/disease/lung-cancer/map2k1/ (Updated June 18). In some embodiments described herein, a MEK1 mutation is named based on the resulting amino acid substitution/deletion/frameshift according to a human MEK1 protein, e.g., as set forth in MPKKKPTPIQLNPAPDGSAVNGTSSAETNLEALQKKLEELELDEQQRKRLEAFLTQKQK VGELKDDDFEKISELGAGNGGVVFKVSHKPSGLVMARKLIHLEIKPAIRNQIIRELQVLH ECNSPYIVGFYGAFYSDGEISICMEHMDGGSLDQVLKKAGRIPEQILGKVSIAVIKGLTYL REKHKIMHRDVKPSNILVNSRGEIKLCDFGVSGQLIDSMANSFVGTRSYMSPERLQGTH YSVQSDIWSMGLSLVEMAVGRYPIPPPDAKELELMFGCQVEGDAAETPPRPRTPGRPLS SYGMDSRPPMAIFELLDYIVNEPPPKLPSGVFSLEFQDFVNKCLIKNPAERADLKQLMVH AFIKRSDAEEVDFAGWLCSTIGLNQPSTPTHAAGV (SEQ ID NO:332). An exemplary human MEK1 cDNA sequence is set forth in ATGCCCAAGAAGAAGCCGACGCCCATCCAGCTGAACCCGGCCCCCGACGGCTCTGC AGTTAACGGGACCAGCTCTGCGGAGACCAACTTGGAGGCCTTGCAGAAGAAGCTGG AGGAGCTAGAGCTTGATGAGCAGCAGCGAAAGCGCCTTGAGGCCTTTCTTACCCAG AAGCAGAAGGTGGGAGAACTGAAGGATGACGACTTTGAGAAGATCAGTGAGCTGG GGGCTGGCAATGGCGGTGTGGTGTTCAAGGTCTCCCACAAGCCTTCTGGCCTGGTCA TGGCCAGAAAGCTAATTCATCTGGAGATCAAACCCGCAATCCGGAACCAGATCATA AGGGAGCTGCAGGTTCTGCATGAGTGCAACTCTCCGTACATCGTGGGCTTCTATGGT GCGTTCTACAGCGATGGCGAGATCAGTATCTGCATGGAGCACATGGATGGAGGTTC TCTGGATCAAGTCCTGAAGAAAGCTGGAAGAATTCCTGAACAAATTTTAGGAAAAG TTAGCATTGCTGTAATAAAAGGCCTGACATATCTGAGGGAGAAGCACAAGATCATG CACAGAGATGTCAAGCCCTCCAACATCCTAGTCAACTCCCGTGGGGAGATCAAGCT CTGTGACTTTGGGGTCAGCGGGCAGCTCATCGACTCCATGGCCAACTCCTTCGTGGG CACAAGGTCCTACATGTCGCCAGAAAGACTCCAGGGGACTCATTACTCTGTGCAGTC AGACATCTGGAGCATGGGACTGTCTCTGGTAGAGATGGCGGTTGGGAGGTATCCCA TCCCTCCTCCAGATGCCAAGGAGCTGGAGCTGATGTTTGGGTGCCAGGTGGAAGGA GATGCGGCTGAGACCCCACCCAGGCCAAGGACCCCCGGGAGGCCCCTTAGCTCATA CGGAATGGACAGCCGACCTCCCATGGCAATTTTTGAGTTGTTGGATTACATAGTCAA CGAGCCTCCTCCAAAACTGCCCAGTGGAGTGTTCAGTCTGGAATTTCAAGATTTTGT GAATAAATGCTTAATAAAAAACCCCGCAGAGAGAGCAGATTTGAAGCAACTCATGG TTCATGCTTTTATCAAGAGATCTGATGCTGAGGAAGTGGATTTTGCAGGTTGGCTCT GCTCCACCATCGGCCTTAACCAGCCCAGCACACCAACCCATGCTGCTGGCGTCTAA (SEQ ID NO:345). In some embodiments, a DNA mutation results in the mutation of Q56 or K57 according to SEQ ID NO:332 or SEQ ID NO:345. For example, in some embodiments, a DNA mutation in a MEK1 gene encodes or results in a K57N mutated MEK1 protein (numbering according to SEQ ID NO:332). These DNA mutations are also described by their nucleotide positions (rather than mutated polypeptide codons) in Table A1 infra. For example, in some embodiments, a DNA mutation in a MEK1 gene results in a c.171G>T mutation in the corresponding cDNA sequence of SEQ ID NO:345.

In some embodiments, a primer pair for amplifying the locus of a MEK1 mutation (e.g., encoding or resulting in a K57N mutated MEK1 protein) comprises the sequences CCTTCAGTTCTCCCACCTTCTG (SEQ ID NO:398).

In some embodiments, the methods of the present disclosure include amplifying the loci of one or more mutations (e.g., DNA mutations) in a HER2 gene. HER2 encodes the HER2/neu proto-oncogene, a receptor tyrosine kinase frequently mutated in human cancers, also known as ERBB2, HER-2, CD340, MLN19, NEU, NGL, and TKR1. In some embodiments, the HER2 gene is a human HER2 gene. In some embodiments, a human HER2 gene refers to the gene described by NCBI Entrez Gene ID No. 2064, including mutants and variants thereof. In other embodiments, the HER2 gene is from one of the following organisms: mouse (see, e.g., NCBI Entrez Gene ID No. 13866), rat (see, e.g., NCBI Entrez Gene ID No. 24337), fish (see, e.g., NCBI Entrez Gene ID No. 30300), dog (see, e.g., NCBI Entrez Gene ID No. 403883), horse (see, e.g., NCBI Entrez Gene ID No. 100054739), chimpanzee (see, e.g., NCBI Entrez Gene ID No. 454636), or cat (see, e.g., NCBI Entrez Gene ID No. 751824).

A variety of HER2 mutations associated with cancer are known and may be suitably detected by the methods described herein; see, e.g., Loyly, C., L. Horn, W. Pao. 2015. HER2 (ERBB2) in Non-Small Cell Lung Cancer (NSCLC). My Cancer Genome at www.mycancergenome.org/content/disease/lung-cancer/erbb2/ (Updated June 18). Alterations in ERBB2 found in cancer also include insertions in the kinase domain or deletions in the extracellular domain. Large deletions in the extracellular domain of ERBB2 result in mutant products p95HER2 and A16HER2. Other commonly mutated residues include G309, S310, S335, L755, 777, and 842. HER2 activation is associated with poor prognosis in a number of cancer types, including NSCLC with co-expression of EGFR. After EGFR T790M, HER2 and MET amplifications are the most common findings of acquired resistance (10-20%) under first-generation EGFR TKIs in NSLCs. FDA approved drugs sensitive to ERBB2 include trastuzumab, afatinib, lapatinib, and pertuzumab. The ratio of T790M/activating-mutations may predict the patients who will remain sensitive to third-generation TKIs longer. HER2+ status is associated with resistance to endocrine and chemotherapy regimens. Alterations, including 95HER2, A16HER2, L726, L755, P780, and small insertions in exon 20, are resistant to trastuzumab or lapatinib. In order to overcome resistance to first-generation EGFR/HER inhibitors, the second-generation EGFR/HER-TKIs, including afatinib, dacomitinib, and neratinib, irreversibly block enzymatic activation of EGFR, HER2, and HER4. In some embodiments described herein, a HER2 mutation is named based on the resulting amino acid substitution/deletion/frameshift according to a human HER2 protein, e.g., as set forth in MELAALCRWGLLLALLPPGAASTQVCTGTDMKLRLPASPETHLDMLRHLYQGCQVVQ GNLELTYLPTNASLSFLQDIQEVQGYVLIAHNQVRQVPLQRLRIVRGTQLFEDNYALAV LDNGDPLNNTTPVTGASPGGLRELQLRSLTEILKGGVLIQRNPQLCYQDTILWKDIFHKN NQLALTLIDTNRSRACHPCSPMCKGSRCWGESSEDCQSLTRTVCAGGCARCKGPLPTDC CHEQCAAGCTGPKHSDCLACLHFNHSGICELHCPALVTYNTDTFESMPNPEGRYTFGAS CVTACPYNYLSTDVGSCTLVCPLHNQEVTAEDGTQRCEKCSKPCARVCYGLGMEHLRE VRAVTSANIQEFAGCKKIFGSLAFLPESFDGDPASNTAPLQPEQLQVFETLEEITGYLYIS AWPDSLPDLSVFQNLQVIRGRILHNGAYSLTLQGLGISWLGLRSLRELGSGLALIHHNTH LCFVHTVPWDQLFRNPHQALLHTANRPEDECVGEGLACHQLCARGHCWGPGPTQCVN CSQFLRGQECVEECRVLQGLPREYVNARHCLPCHPECQPQNGSVTCFGPEADQCVACA HYKDPPFCVARCPSGVKPDLSYMPIWKFPDEEGACQPCPINCTHSCVDLDDKGCPAEQR ASPLTSIISAVVGILLVVVLGVVFGILIKRRQQKIRKYTMRRLLQETELVEPLTPSGAMPN QAQMRILKETELRKVKVLGSGAFGTVYKGIWIPDGENVKIPVAIKVLRENTSPKANKEIL DEAYVMAGVGSPYVSRLLGICLTSTVQLVTQLMPYGCLLDHVRENRGRLGSQDLLNW CMQIAKGMSYLEDVRLVHRDLAARNVLVKSPNHVKITDFGLARLLDIDETEYHADGGK VPIKWMALESILRRRFTHQSDVWSYGVTVWELMTFGAKPYDGIPAREIPDLLEKGERLP QPPICTIDVYMIMVKCWMIDSECRPRFRELVSEFSRMARDPQRFVVIQNEDLGPASPLDS TFYRSLLEDDDMGDLVDAEEYLVPQQGFFCPDPAPGAGGMVHHRHRSSSTRNM (SEQ ID NO:333). An exemplary human HER2 cDNA sequence is set forth in ATGGAGCTGGCGGCCTTGTGCCGCTGGGGGCTCCTCCTCGCCCTCTTGCCCCCCGGA GCCGCGAGCACCCAAGTGTGCACCGGCACAGACATGAAGCTGCGGCTCCCTGCCAG TCCCGAGACCCACCTGGACATGCTCCGCCACCTCTACCAGGGCTGCCAGGTGGTGCA GGGAAACCTGGAACTCACCTACCTGCCCACCAATGCCAGCCTGTCCTTCCTGCAGGA TATCCAGGAGGTGCAGGGCTACGTGCTCATCGCTCACAACCAAGTGAGGCAGGTCC CACTGCAGAGGCTGCGGATTGTGCGAGGCACCCAGCTCTTTGAGGACAACTATGCC CTGGCCGTGCTAGACAATGGAGACCCGCTGAACAATACCACCCCTGTCACAGGGGC CTCCCCAGGAGGCCTGCGGGAGCTGCAGCTTCGAAGCCTCACAGAGATCTTGAAAG GAGGGGTCTTGATCCAGCGGAACCCCCAGCTCTGCTACCAGGACACGATTTTGTGGA AGGACATCTTCCACAAGAACAACCAGCTGGCTCTCACACTGATAGACACCAACCGC TCTCGGGCCTGCCACCCCTGTTCTCCGATGTGTAAGGGCTCCCGCTGCTGGGGAGAG AGTTCTGAGGATTGTCAGAGCCTGACGCGCACTGTCTGTGCCGGTGGCTGTGCCCGC TGCAAGGGGCCACTGCCCACTGACTGCTGCCATGAGCAGTGTGCTGCCGGCTGCAC GGGCCCCAAGCACTCTGACTGCCTGGCCTGCCTCCACTTCAACCACAGTGGCATCTG TGAGCTGCACTGCCCAGCCCTGGTCACCTACAACACAGACACGTTTGAGTCCATGCC CAATCCCGAGGGCCGGTATACATTCGGCGCCAGCTGTGTGACTGCCTGTCCCTACAA CTACCTTTCTACGGACGTGGGATCCTGCACCCTCGTCTGCCCCCTGCACAACCAAGA GGTGACAGCAGAGGATGGAACACAGCGGTGTGAGAAGTGCAGCAAGCCCTGTGCCC GAGTGTGCTATGGTCTGGGCATGGAGCACTTGCGAGAGGTGAGGGCAGTTACCAGT GCCAATATCCAGGAGTTTGCTGGCTGCAAGAAGATCTTTGGGAGCCTGGCATTTCTG CCGGAGAGCTTTGATGGGGACCCAGCCTCCAACACTGCCCCGCTCCAGCCAGAGCA GCTCCAAGTGTTTGAGACTCTGGAAGAGATCACAGGTTACCTATACATCTCAGCATG GCCGGACAGCCTGCCTGACCTCAGCGTCTTCCAGAACCTGCAAGTAATCCGGGGAC GAATTCTGCACAATGGCGCCTACTCGCTGACCCTGCAAGGGCTGGGCATCAGCTGGC TGGGGCTGCGCTCACTGAGGGAACTGGGCAGTGGACTGGCCCTCATCCACCATAAC ACCCACCTCTGCTTCGTGCACACGGTGCCCTGGGACCAGCTCTTTCGGAACCCGCAC CAAGCTCTGCTCCACACTGCCAACCGGCCAGAGGACGAGTGTGTGGGCGAGGGCCT GGCCTGCCACCAGCTGTGCGCCCGAGGGCACTGCTGGGGTCCAGGGCCCACCCAGT GTGTCAACTGCAGCCAGTTCCTTCGGGGCCAGGAGTGCGTGGAGGAATGCCGAGTA CTGCAGGGGCTCCCCAGGGAGTATGTGAATGCCAGGCACTGTTTGCCGTGCCACCCT GAGTGTCAGCCCCAGAATGGCTCAGTGACCTGTTTTGGACCGGAGGCTGACCAGTGT GTGGCCTGTGCCCACTATAAGGACCCTCCCTTCTGCGTGGCCCGCTGCCCCAGCGGT GTGAAACCTGACCTCTCCTACATGCCCATCTGGAAGTTTCCAGATGAGGAGGGCGCA TGCCAGCCTTGCCCCATCAACTGCACCCACTCCTGTGTGGACCTGGATGACAAGGGC TGCCCCGCCGAGCAGAGAGCCAGCCCTCTGACGTCCATCATCTCTGCGGTGGTTGGC ATTCTGCTGGTCGTGGTCTTGGGGGTGGTCTTTGGGATCCTCATCAAGCGACGGCAG CAGAAGATCCGGAAGTACACGATGCGGAGACTGCTGCAGGAAACGGAGCTGGTGG AGCCGCTGACACCTAGCGGAGCGATGCCCAACCAGGCGCAGATGCGGATCCTGAAA GAGACGGAGCTGAGGAAGGTGAAGGTGCTTGGATCTGGCGCTTTTGGCACAGTCTA CAAGGGCATCTGGATCCCTGATGGGGAGAATGTGAAAATTCCAGTGGCCATCAAAG TGTTGAGGGAAAACACATCCCCCAAAGCCAACAAAGAAATCTTAGACGAAGCATAC GTGATGGCTGGTGTGGGCTCCCCATATGTCTCCCGCCTTCTGGGCATCTGCCTGACA TCCACGGTGCAGCTGGTGACACAGCTTATGCCCTATGGCTGCCTCTTAGACCATGTC CGGGAAAACCGCGGACGCCTGGGCTCCCAGGACCTGCTGAACTGGTGTATGCAGAT TGCCAAGGGGATGAGCTACCTGGAGGATGTGCGGCTCGTACACAGGGACTTGGCCG CTCGGAACGTGCTGGTCAAGAGTCCCAACCATGTCAAAATTACAGACTTCGGGCTG GCTCGGCTGCTGGACATTGACGAGACAGAGTACCATGCAGATGGGGGCAAGGTGCC CATCAAGTGGATGGCGCTGGAGTCCATTCTCCGCCGGCGGTTCACCCACCAGAGTGA TGTGTGGAGTTATGGTGTGACTGTGTGGGAGCTGATGACTTTTGGGGCCAAACCTTA CGATGGGATCCCAGCCCGGGAGATCCCTGACCTGCTGGAAAAGGGGGAGCGGCTGC CCCAGCCCCCCATCTGCACCATTGATGTCTACATGATCATGGTCAAATGTTGGATGA TTGACTCTGAATGTCGGCCAAGATTCCGGGAGTTGGTGTCTGAATTCTCCCGCATGG CCAGGGACCCCCAGCGCTTTGTGGTCATCCAGAATGAGGACTTGGGCCCAGCCAGT CCCTTGGACAGCACCTTCTACCGCTCACTGCTGGAGGACGATGACATGGGGGACCTG GTGGATGCTGAGGAGTATCTGGTACCCCAGCAGGGCTTCTTCTGTCCAGACCCTGCC CCGGGCGCTGGGGGCATGGTCCACCACAGGCACCGCAGCTCATCTACCAGGAATAT GTGA (SEQ ID NO:346). In some embodiments, a DNA mutation results in the mutation of A775 or G776 according to SEQ ID NO:333 or SEQ ID NO:346. For example, in some embodiments, a DNA mutation in a HER2 gene encodes or results in a A775_G776insYVMA mutated HER2 protein (numbering according to SEQ ID NO:333). These DNA mutations are also described by their nucleotide positions (rather than mutated polypeptide codons) in Table A1 infra. For example, in some embodiments, a DNA mutation in a HER2 gene results in a c.2324_2325ins12 mutation in the corresponding cDNA sequence of SEQ ID NO:346.

In some embodiments, a primer pair for amplifying the locus of a HER2 mutation (e.g., encoding or resulting in a A775_G776insYVMA mutated HER2 protein) comprises the sequences ATGGCTGTGGTTTGTGATGGT (SEQ ID NO:414) and ACACCAGCCATCACGTAAGACA (SEQ ID NO:415), respectively.

Exemplary and non-limiting DNA mutations are provided in Table A below.

In some embodiments, the methods of the present disclosure include amplifying the loci of one or more mutations (e.g., RNA mutations) in an ALK gene. ALK encodes the anaplastic lymphoma kinase, a receptor tyrosine kinase frequently mutated in human cancers, also known as CD246, NBLST3, or the ALK tyrosine kinase receptor. In some embodiments, the ALK gene is a human ALK gene. In some embodiments, a human ALK gene refers to the gene described by NCBI Entrez Gene ID No. 238, including mutants and variants thereof. In other embodiments, the ALK gene is from one of the following organisms: mouse (see, e.g., NCBI Entrez Gene ID No. 11682), rat (see, e.g., NCBI Entrez Gene ID No. 266802), fish (see, e.g., NCBI Entrez Gene ID No. 563509), cattle (see, e.g., NCBI Entrez Gene ID No. 536642), chicken (see, e.g., NCBI Entrez Gene ID No. 421297), or chimpanzee (see, e.g., NCBI Entrez Gene ID No. 459127).

A variety of ALK mutations associated with cancer are known and may be suitably detected by the methods described herein; see, e.g., Loyly, C., L. Horn, W. Pao. 2015. ALK in Non-Small Cell Lung Cancer (NSCLC). My Cancer Genome at www.mycancergenome.org/content/disease/lung-cancer/alk/ (Updated September 29). The ALK gene, on chromosome 2, encodes a receptor tyrosine kinase involved in cell growth, transformation, and differentiation. Alterations in ALK constitutively activate the kinase regulating the JAK-STAT3, PI3K-AKT and RAS-MAPK pathways and driving tumorigenesis in various tissues. The most common ALK alterations are gene rearrangements detectable by fluorescence in situ hybridization (FISH). In addition to fusions, various cancers harbor gain of function mutations in ALK, such as F1174L, D1091N, 11250T, and R1275. ALK-rearranged NSCLC represents 3-7% of all NSCLC. Eight percent of ALK-rearranged NSCLC are also EGFR+ or KRAS+ mutated. ALK rearrangements are associated with response to crizotinib in approximately 60-70% of ALK+ patients. A number of point mutations, such as the F1174L, are known to be associated with resistance to ALK inhibitor therapy. Additionally, ALK copy number gain as well as activating mutations in other driver genes such as EGFR may be acquired resistance mechanisms in patients undergoing ALK inhibitor therapy. FDA approved drugs sensitive to ALK against NSCLC include ceritinib, alectinib, and crizotinib. Evidence suggests differential primary response to crizotinib depending on the ALK fusion partner in NSCLC. Heat shock protein 90 (HSP90) inhibitors present a potential line of treatment due to dependence of ALK fusions, such as EML4-ALK, on HSP90 for stability. Next-generation agents such as alectinib may salvage CNS metastasis in ALK+ patients treated with both crizotinib and ceritinib. In some embodiments described herein, an ALK mutation is named based on the resulting amino acid substitution/deletion/frameshift/translocation according to a human ALK protein, e.g., as set forth in MGAIGLLWLLPLLLSTAAVGSGMGTGQRAGSPAAGPPLQPREPLSYSRLQRKSLAVDFV VPSLFRVYARDLLLPPSSSELKAGRPEARGSLALDCAPLLRLLGPAPGVSWTAGSPAPAE ARTLSRVLKGGSVRKLRRAKQLVLELGEEAILEGCVGPPGEAAVGLLQFNLSELFSWWI RQGEGRLRIRLMPEKKASEVGREGRLSAAIRASQPRLLFQIFGTGHSSLESPTNMPSPSPD YFTWNLTWIMKDSFPFLSHRSRYGLECSFDFPCELEYSPPLHDLRNQSWSWRRIPSEEAS QMDLLDGPGAERSKEMPRGSFLLLNTSADSKHTILSPWMRSSSEHCTLAVSVHRHLQPS GRYIAQLLPHNEAAREILLMPTPGKHGWTVLQGRIGRPDNPFRVALEYISSGNRSLSAVD FFALKNCSEGTSPGSKMALQSSFTCWNGTVLQLGQACDFHQDCAQGEDESQMCRKLPV GFYCNFEDGFCGWTQGTLSPHTPQWQVRTLKDARFQDHQDHALLLSTTDVPASESATV TSATFPAPIKSSPCELRMSWLIRGVLRGNVSLVLVENKTGKEQGRMVWHVAAYEGLSL WQWMVLPLLDVSDRFWLQMVAWWGQGSRAIVAFDNISISLDCYLTISGEDKILQNTAP KSRNLFERNPNKELKPGENSPRQTPIFDPTVHWLFTTCGASGPHGPTQAQCNNAYQNSN LSVEVGSEGPLKGIQIWKVPATDTYSISGYGAAGGKGGKNTMMRSHGVSVLGIFNLEKD DMLYILVGQQGEDACPSTNQLIQKVCIGENNVIEEEIRVNRSVHEWAGGGGGGGGATY VFKMKDGVPVPLIIAAGGGGRAYGAKTDTFHPERLENNSSVLGLNGNSGAAGGGGGW NDNTSLLWAGKSLQEGATGGHSCPQAMKKWGWETRGGFGGGGGGCSSGGGGGGYIG GNAASNNDPEMDGEDGVSFISPLGILYTPALKVMEGHGEVNIKHYLNCSHCEVDECHM DPESHKVICFCDHGTVLAEDGVSCIVSPTPEPHLPLSLILSVVTSALVAALVLAFSGIMIVY RRKHQELQAMQMELQSPEYKLSKLRTSTIMTDYNPNYCFAGKTSSISDLKEVPRKNITLI RGLGHGAFGEVYEGQVSGMPNDPSPLQVAVKTLPEVCSEQDELDFLMEALIISKFNHQN IVRCIGVSLQSLPRFILLELMAGGDLKSFLRETRPRPSQPSSLAMLDLLHVARDIACGCQY LEENHFIHRDIAARNCLLTCPGPGRVAKIGDFGMARDIYRASYYRKGGCAMLPVKWMP PEAFMEGIFTSKTDTWSFGVLLWEIFSLGYMPYPSKSNQEVLEFVTSGGRMDPPKNCPGP VYRIMTQCWQHQPEDRPNFAIILERIEYCTQDPDVINTALPIEYGPLVEEEEKVPVRPKDP EGVPPLLVSQQAKREEERSPAAPPPLPTTSSGKAAKKPTAAEISVRVPRGPAVEGGHVN MAFSQSNPPSELHKVHGSRNKPTSLWNPTYGSWFTEKPTKKNNPIAKKEPHDRGNLGLE GSCTVPPNVATGRLPGASLLLEPSSLTANMKEVPLFRLRHFPCGNVNYGYQQQGLPLEA ATAPGAGHYEDTILKSKNSMNQPGP (SEQ ID NO:334). An exemplary human ALK cDNA sequence is set forth in ATGGGAGCCATCGGGCTCCTGTGGCTCCTGCCGCTGCTGCTTTCCACGGCAGCTGTG GGCTCCGGGATGGGGACCGGCCAGCGCGCGGGCTCCCCAGCTGCGGGGCCGCCGCT GCAGCCCCGGGAGCCACTCAGCTACTCGCGCCTGCAGAGGAAGAGTCTGGCAGTTG ACTTCGTGGTGCCCTCGCTCTTCCGTGTCTACGCCCGGGACCTACTGCTGCCACCATC CTCCTCGGAGCTGAAGGCTGGCAGGCCCGAGGCCCGCGGCTCGCTAGCTCTGGACT GCGCCCCGCTGCTCAGGTTGCTGGGGCCGGCGCCGGGGGTCTCCTGGACCGCCGGTT CACCAGCCCCGGCAGAGGCCCGGACGCTGTCCAGGGTGCTGAAGGGCGGCTCCGTG CGCAAGCTCCGGCGTGCCAAGCAGTTGGTGCTGGAGCTGGGCGAGGAGGCGATCTT GGAGGGTTGCGTCGGGCCCCCCGGGGAGGCGGCTGTGGGGCTGCTCCAGTTCAATC TCAGCGAGCTGTTCAGTTGGTGGATTCGCCAAGGCGAAGGGCGACTGAGGATCCGC CTGATGCCCGAGAAGAAGGCGTCGGAAGTGGGCAGAGAGGGAAGGCTGTCCGCGG CAATTCGCGCCTCCCAGCCCCGCCTTCTCTTCCAGATCTTCGGGACTGGTCATAGCTC CTTGGAATCACCAACAAACATGCCTTCTCCTTCTCCTGATTATTTTACATGGAATCTC ACCTGGATAATGAAAGACTCCTTCCCTTTCCTGTCTCATCGCAGCCGATATGGTCTG GAGTGCAGCTTTGACTTCCCCTGTGAGCTGGAGTATTCCCCTCCACTGCATGACCTC AGGAACCAGAGCTGGTCCTGGCGCCGCATCCCCTCCGAGGAGGCCTCCCAGATGGA CTTGCTGGATGGGCCTGGGGCAGAGCGTTCTAAGGAGATGCCCAGAGGCTCCTTTCT CCTTCTCAACACCTCAGCTGACTCCAAGCACACCATCCTGAGTCCGTGGATGAGGAG CAGCAGTGAGCACTGCACACTGGCCGTCTCGGTGCACAGGCACCTGCAGCCCTCTG GAAGGTACATTGCCCAGCTGCTGCCCCACAACGAGGCTGCAAGAGAGATCCTCCTG ATGCCCACTCCAGGGAAGCATGGTTGGACAGTGCTCCAGGGAAGAATCGGGCGTCC AGACAACCCATTTCGAGTGGCCCTGGAATACATCTCCAGTGGAAACCGCAGCTTGTC TGCAGTGGACTTCTTTGCCCTGAAGAACTGCAGTGAAGGAACATCCCCAGGCTCCAA GATGGCCCTGCAGAGCTCCTTCACTTGTTGGAATGGGACAGTCCTCCAGCTTGGGCA GGCCTGTGACTTCCACCAGGACTGTGCCCAGGGAGAAGATGAGAGCCAGATGTGCC GGAAACTGCCTGTGGGTTTTTACTGCAACTTTGAAGATGGCTTCTGTGGCTGGACCC AAGGCACACTGTCACCCCACACTCCTCAATGGCAGGTCAGGACCCTAAAGGATGCC CGGTTCCAGGACCACCAAGACCATGCTCTATTGCTCAGTACCACTGATGTCCCCGCT TCTGAAAGTGCTACAGTGACCAGTGCTACGTTTCCTGCACCGATCAAGAGCTCTCCA TGTGAGCTCCGAATGTCCTGGCTCATTCGTGGAGTCTTGAGGGGAAACGTGTCCTTG GTGCTAGTGGAGAACAAAACCGGGAAGGAGCAAGGCAGGATGGTCTGGCATGTCG CCGCCTATGAAGGCTTGAGCCTGTGGCAGTGGATGGTGTTGCCTCTCCTCGATGTGT CTGACAGGTTCTGGCTGCAGATGGTCGCATGGTGGGGACAAGGATCCAGAGCCATC GTGGCTTTTGACAATATCTCCATCAGCCTGGACTGCTACCTCACCATTAGCGGAGAG GACAAGATCCTGCAGAATACAGCACCCAAATCAAGAAACCTGTTTGAGAGAAACCC AAACAAGGAGCTGAAACCCGGGGAAAATTCACCAAGACAGACCCCCATCTTTGACC CTACAGTTCATTGGCTGTTCACCACATGTGGGGCCAGCGGGCCCCATGGCCCCACCC AGGCACAGTGCAACAACGCCTACCAGAACTCCAACCTGAGCGTGGAGGTGGGGAGC GAGGGCCCCCTGAAAGGCATCCAGATCTGGAAGGTGCCAGCCACCGACACCTACAG CATCTCGGGCTACGGAGCTGCTGGCGGGAAAGGCGGGAAGAACACCATGATGCGGT CCCACGGCGTGTCTGTGCTGGGCATCTTCAACCTGGAGAAGGATGACATGCTGTACA TCCTGGTTGGGCAGCAGGGAGAGGACGCCTGCCCCAGTACAAACCAGTTAATCCAG AAAGTCTGCATTGGAGAGAACAATGTGATAGAAGAAGAAATCCGTGTGAACAGAA GCGTGCATGAGTGGGCAGGAGGCGGAGGAGGAGGGGGTGGAGCCACCTACGTATTT AAGATGAAGGATGGAGTGCCGGTGCCCCTGATCATTGCAGCCGGAGGTGGTGGCAG GGCCTACGGGGCCAAGACAGACACGTTCCACCCAGAGAGACTGGAGAATAACTCCT CGGTTCTAGGGCTAAACGGCAATTCCGGAGCCGCAGGTGGTGGAGGTGGCTGGAAT GATAACACTTCCTTGCTCTGGGCCGGAAAATCTTTGCAGGAGGGTGCCACCGGAGG ACATTCCTGCCCCCAGGCCATGAAGAAGTGGGGGTGGGAGACAAGAGGGGGTTTCG GAGGGGGTGGAGGGGGGTGCTCCTCAGGTGGAGGAGGCGGAGGATATATAGGCGG CAATGCAGCCTCAAACAATGACCCCGAAATGGATGGGGAAGATGGGGTTTCCTTCA TCAGTCCACTGGGCATCCTGTACACCCCAGCTTTAAAAGTGATGGAAGGCCACGGG GAAGTGAATATTAAGCATTATCTAAACTGCAGTCACTGTGAGGTAGACGAATGTCA CATGGACCCTGAAAGCCACAAGGTCATCTGCTTCTGTGACCACGGGACGGTGCTGG CTGAGGATGGCGTCTCCTGCATTGTGTCACCCACCCCGGAGCCACACCTGCCACTCT CGCTGATCCTCTCTGTGGTGACCTCTGCCCTCGTGGCCGCCCTGGTCCTGGCTTTCTC CGGCATCATGATTGTGTACCGCCGGAAGCACCAGGAGCTGCAAGCCATGCAGATGG AGCTGCAGAGCCCTGAGTACAAGCTGAGCAAGCTCCGCACCTCGACCATCATGACC GACTACAACCCCAACTACTGCTTTGCTGGCAAGACCTCCTCCATCAGTGACCTGAAG GAGGTGCCGCGGAAAAACATCACCCTCATTCGGGGTCTGGGCCATGGCGCCTTTGG GGAGGTGTATGAAGGCCAGGTGTCCGGAATGCCCAACGACCCAAGCCCCCTGCAAG TGGCTGTGAAGACGCTGCCTGAAGTGTGCTCTGAACAGGACGAACTGGATTTCCTCA TGGAAGCCCTGATCATCAGCAAATTCAACCACCAGAACATTGTTCGCTGCATTGGGG TGAGCCTGCAATCCCTGCCCCGGTTCATCCTGCTGGAGCTCATGGCGGGGGGAGACC TCAAGTCCTTCCTCCGAGAGACCCGCCCTCGCCCGAGCCAGCCCTCCTCCCTGGCCA TGCTGGACCTTCTGCACGTGGCTCGGGACATTGCCTGTGGCTGTCAGTATTTGGAGG AAAACCACTTCATCCACCGAGACATTGCTGCCAGAAACTGCCTCTTGACCTGTCCAG GCCCTGGAAGAGTGGCCAAGATTGGAGACTTCGGGATGGCCCGAGACATCTACAGG GCGAGCTACTATAGAAAGGGAGGCTGTGCCATGCTGCCAGTTAAGTGGATGCCCCC AGAGGCCTTCATGGAAGGAATATTCACTTCTAAAACAGACACATGGTCCTTTGGAGT GCTGCTATGGGAAATCTTTTCTCTTGGATATATGCCATACCCCAGCAAAAGCAACCA GGAAGTTCTGGAGTTTGTCACCAGTGGAGGCCGGATGGACCCACCCAAGAACTGCC CTGGGCCTGTATACCGGATAATGACTCAGTGCTGGCAACATCAGCCTGAAGACAGG CCCAACTTTGCCATCATTTTGGAGAGGATTGAATACTGCACCCAGGACCCGGATGTA ATCAACACCGCTTTGCCGATAGAATATGGTCCACTTGTGGAAGAGGAAGAGAAAGT GCCTGTGAGGCCCAAGGACCCTGAGGGGGTTCCTCCTCTCCTGGTCTCTCAACAGGC AAAACGGGAGGAGGAGCGCAGCCCAGCTGCCCCACCACCTCTGCCTACCACCTCCT CTGGCAAGGCTGCAAAGAAACCCACAGCTGCAGAGATCTCTGTTCGAGTCCCTAGA GGGCCGGCCGTGGAAGGGGGACACGTGAATATGGCATTCTCTCAGTCCAACCCTCC TTCGGAGTTGCACAAGGTCCACGGATCCAGAAACAAGCCCACCAGCTTGTGGAACC CAACGTACGGCTCCTGGTTTACAGAGAAACCCACCAAAAAGAATAATCCTATAGCA AAGAAGGAGCCACACGACAGGGGTAACCTGGGGCTGGAGGGAAGCTGTACTGTCCC ACCTAACGTTGCAACTGGGAGACTTCCGGGGGCCTCACTGCTCCTAGAGCCCTCTTC GCTGACTGCCAATATGAAGGAGGTACCTCTGTTCAGGCTACGTCACTTCCCTTGTGG GAATGTCAATTACGGCTACCAGCAACAGGGCTTGCCCTTAGAAGCCGCTACTGCCCC TGGAGCTGGTCATTACGAGGATACCATTCTGAAAAGCAAGAATAGCATGAACCAGC CTGGGCCCTGA (SEQ ID NO:347). In some embodiments, an RNA mutation results in a translocation, gene rearrangement, or fusion gene at the ALK locus. In some embodiments, an RNA mutation results in a fusion between the ALK and EML4 genes. For example, in some embodiments, an RNA mutation in an ALK gene encodes or results in an E13;A20, E20;A20, E6a;A20, E6b;A20 ALK fusion protein. As used herein, nomenclature for a fusion gene (e.g., any of the RNA mutations involving fusion genes described herein) can use the following formats interchangeably: GENE1 E #: GENE2 E # (e.g., EML E13:ALK E20) and GENE1 #;GENE2 # (e.g., E13;A20), referring to both genes and the respective gene exon numbers involved.

In some embodiments, a primer pair for amplifying the locus of an ALK mutation (e.g., encoding or resulting in an EML4:ALK fusion protein) comprises one sequence (e.g., that hybridizes with an EML4-specific locus of the fusion gene) selected from the group consisting of TATGGAGCAAAACTACTGTAGAGCC (SEQ ID NO:357), CCAGCTACATCACACACCTTGACT (SEQ ID NO:358), TAATACCAAAAGTTACCAAAACTGCA (SEQ ID NO:359), CAATCTCTGAAGATCATGTGGCC (SEQ ID NO:360), CAAGTGGCACAGTGGTGGC (SEQ ID NO:361), and TAACTGGAGGAGGGAAAGACAGA (SEQ ID NO:362); and another sequence (e.g., that hybridizes with an ALK-specific locus of the fusion gene) selected from the group consisting of AGTTGGGGTTGTAGTCGGTCAT (SEQ ID NO:363) and GAAGCCTCCCTGGATCTCC (SEQ ID NO:364).

In some embodiments, the methods of the present disclosure include amplifying the loci of one or more mutations (e.g., RNA mutations) in an ROS gene. ROS encodes the c-ros proto-oncogene, a receptor tyrosine kinase frequently mutated in human cancers, also known as ROS1, MCF3, and c-ros-1. In some embodiments, the ROS gene is a human ROS gene. In some embodiments, a human ROS gene refers to the gene described by NCBI Entrez Gene ID No. 6098, including mutants and variants thereof. In other embodiments, the ROS gene is from one of the following organisms: mouse (see, e.g., NCBI Entrez Gene ID No. 19886), rat (see, e.g., NCBI Entrez Gene ID No. 25346), fish (see, e.g., NCBI Entrez Gene ID No. 245951), cattle (see, e.g., NCBI Entrez Gene ID No. 100336768), chicken (see, e.g., NCBI Entrez Gene ID No. 396192), or chimpanzee (see, e.g., NCBI Entrez Gene ID No. 472108).

A variety of ROS mutations associated with cancer are known and may be suitably detected by the methods described herein; see, e.g., Loyly, C., L. Horn, W. Pao. 2015. ROS1 in Non-Small Cell Lung Cancer (NSCLC). My Cancer Genome at www.mycancergenome.org/content/disease/lung-cancer/ros1/ (Updated November 17). A number of ROS1 fusions, detectable by FISH, have been identified in 1-2% of NSCLC: FIG-ROS1, SLC34A2-ROS1, CD74-ROS1, LRIG3-ROS1, KDELR2-ROS1, and CCDC6-ROS1. ROS1 rearrangements share clinical and histological characteristics: never- or light-smoking history, female, younger age, and adenocarcinoma with signet ring cell histology. ALK and ROS1 fusion tumors have a significantly shorter disease free survival, which does not translate into a short overall survival, since patients respond to targeted therapy, such as crizotinib. Two thirds of ROS1+ patients respond to crizotinib, approved in the first-line for NSCLC. Crizotinib resistant ROS1G2032R mutants are sensitive to foretinib and cabozantinib. Patients ultimately develop secondary resistance to crizotinib and later generation therapies. Increased EGFR phosphorylation is detected in 44% of ALK- and ROS1-rearranged crizotinib resistant tumors, indicating that this mechanism may mediate resistance. Ceritinib, ASP3026, and brigatinib exhibit activity against ROS1 kinase, but fail to inhibit crizotinib resistant ROS1. There are a number of multi TKIs in evaluation for resistance to crizotinib, ceritinib, and alectinib: including merestinib, entrectinib, TAE684, and lorlatinib. In some embodiments described herein, an ROS mutation is named based on the resulting amino acid substitution/deletion/frameshift/translocation according to a human ROS protein, e.g., as set forth in MKNIYCLIPKLVNFATLGCLWISVVQCTVLNSCLKSCVTNLGQQLDLGTPHNLSEPCIQG CHFWNSVDQKNCALKCRESCEVGCSSAEGAYEEEVLENADLPTAPFASSIGSHNMTLR WKSANFSGVKYIIQWKYAQLLGSWTYTKTVSRPSYVVKPLHPFTEYIFRVVWIFTAQLQ LYSPPSPSYRTHPHGVPETAPLIRNIESSSPDTVEVSWDPPQFPGGPILGYNLRLISKNQKL DAGTQRTSFQFYSTLPNTIYRFSIAAVNEVGEGPEAESSITTSSSAVQQEEQWLFLSRKTS LRKRSLKHLVDEAHCLRLDAIYHNITGISVDVHQQIVYFSEGTLIWAKKAANMSDVSDL RIFYRGSGLISSISIDWLYQRMYFIMDELVCVCDLENCSNIEEITPPSISAPQKIVADSYNG YVFYLLRDGIYRADLPVPSGRCAEAVRIVESCTLKDFAIKPQAKRIIYFNDTAQVFMSTFL DGSASHLILPRIPFADVKSFACENNDFLVTDGKVIFQQDALSFNEFIVGCDLSHIEEFGFG NLVIFGSSSQLHPLPGRPQELSVLFGSHQALVQWKPPALAIGANVILISDIIELFELGPSAW QNWTYEVKVSTQDPPEVTHIFLNISGTMLNVPELQSAMKYKVSVRASSPKRPGPWSEPS VGTTLVPASEPPFIMAVKEDGLWSKPLNSFGPGEFLSSDIGNVSDMDWYNNSLYYSDTK GDVFVWLLNGTDISENYHLPSIAGAGALAFEWLGHFLYWAGKTYVIQRQSVLTGHTDI VTHVKLLVNDMVVDSVGGYLYWTTLYSVESTRLNGESSLVLQTQPWFSGKKVIALTLD LSDGLLYWLVQDSQCIHLYTAVLRGQSTGDTTITEFAAWSTSEISQNALMYYSGRLFWI NGFRIITTQEIGQKTSVSVLEPARFNQFTIIQTSLKPLPGNFSFTPKVIPDSVQESSFRIEGNA SSFQILWNGPPAVDWGVVFYSVEFSAHSKFLASEQHSLPVFTVEGLEPYALFNLSVTPYT YWGKGPKTSLSLRAPETVPSAPENPRIFILPSGKCCNKNEVVVEFRWNKPKHENGVLTK FEIFYNISNQSITNKTCEDWIAVNVTPSVMSFQLEGMSPRCFIAFQVRAFTSKGPGPYADV VKSTTSEINPFPHLITLLGNKIVFLDMDQNQVVWTFSAERVISAVCYTADNEMGYYAEG DSLFLLHLHNRSSSELFQDSLVFDITVITIDWISRHLYFALKESQNGMQVFDVDLEHKVK YPREVKIHNRNSTIISFSVYPLLSRLYWTEVSNFGYQMFYYSIISHTLHRILQPTATNQQN KRNQCSCNVTEFELSGAMAIDTSNLEKPLIYFAKAQEIWAMDLEGCQCWRVITVPAML AGKTLVSLTVDGDLIYWIITAKDSTQIYQAKKGNGAIVSQVKALRSRHILAYSSVMQPFP DKAFLSLASDTVEPTILNATNTSLTIRLPLAKTNLTWYGITSPTPTYLVYYAEVNDRKNSS DLKYRILEFQDSIALIEDLQPFSTYMIQIAVKNYYSDPLEHLPPGKEIWGKTKNGVPEAVQ LINTFVRSDTSLIISWRESHKPNGPKESVRYQLAISHLALIPETPLRQSEFPNGRLTLLVTR LSGGNIYVLKVLACHSEEMWCTESHPVTVEMFNTPEKPYSLVPENTSLQFNWKAPLNV NLIRFWVELQKWKYNEFYHVKTSCSQGPAYVCNITNLQPYTSYNVRVVVVYKTGENST SLPESFKTKAGVPNKPGIPKLLEGSKNSIQWEKAEDNGCRITYYILEIRKSTSNNLQNQNL RWKMTFNGSCSSVCTWKSKNLKGIFQFRVVAANNLGFGEYSGISENIILVGDDFWIPETS FILTIIVGIFLVVTIPLTFVWHRRLKNQKSAKEGVTVLINEDKELAELRGLAAGVGLANA CYAIHTLPTQEEIENLPAFPREKLTLRLLLGSGAFGEVYEGTAVDILGVGSGEIKVAVKTL KKGSTDQEKIEFLKEAHLMSKFNHPNILKQLGVCLLNEPQYIILELMEGGDLLTYLRKAR MATFYGPLLTLVDLVDLCVDISKGCVYLERMHFIHRDLAARNCLVSVKDYTSPRIVKIG DFGLARDIYKNDYYRKRGEGLLPVRWMAPESLMDGIFTTQSDVWSFGILIWEILTLGHQ PYPAHSNLDVLNYVQTGGRLEPPRNCPDDLWNLMTQCWAQEPDQRPTFHRIQDQLQLF RNFFLNSIYKSRDEANNSGVINESFEGEDGDVICLNSDDIMPVALMETKNREGLNYMVL ATECGQGEEKSEGPLGSQESESCGLRKEEKEPHADKDFCQEKQVAYCPSGKPEGLNYAC LTHSGYGDGSD (SEQ ID NO:335). An exemplary human ROS cDNA sequence is set forth in ATGAAGAACATTTACTGTCTTATTCCGAAGCTTGTCAATTTTGCAACTCTTGGCTGCC TATGGATTTCTGTGGTGCAGTGTACAGTTTTAAATAGCTGCCTAAAGTCGTGTGTAA CTAATCTGGGCCAGCAGCTTGACCTTGGCACACCACATAATCTGAGTGAACCGTGTA TCCAAGGATGTCACTTTTGGAACTCTGTAGATCAGAAAAACTGTGCTTTAAAGTGTC GGGAGTCGTGTGAGGTTGGCTGTAGCAGCGCGGAAGGTGCATATGAAGAGGAAGTA CTGGAAAATGCAGACCTACCAACTGCTCCCTTTGCTTCTTCCATTGGAAGCCACAAT ATGACATTACGATGGAAATCTGCAAACTTCTCTGGAGTAAAATACATCATTCAGTGG AAATATGCACAACTTCTGGGAAGCTGGACTTATACTAAGACTGTGTCCAGACCGTCC TATGTGGTCAAGCCCCTGCACCCCTTCACTGAGTACATTTTCCGAGTGGTTTGGATCT TCACAGCGCAGCTGCAGCTCTACTCCCCTCCAAGTCCCAGTTACAGGACTCATCCTC ATGGAGTTCCTGAAACTGCACCTTTGATTAGGAATATTGAGAGCTCAAGTCCCGACA CTGTGGAAGTCAGCTGGGATCCACCTCAATTCCCAGGTGGACCTATTTTGGGTTATA ACTTAAGGCTGATCAGCAAAAATCAAAAATTAGATGCAGGGACACAGAGAACCAGT TTCCAGTTTTACTCCACTTTACCAAATACTATCTACAGGTTTTCTATTGCAGCAGTAA ATGAAGTTGGTGAGGGTCCAGAAGCAGAATCTAGTATTACCACTTCATCTTCAGCAG TTCAACAAGAGGAACAGTGGCTCTTTTTATCCAGAAAAACTTCTCTAAGAAAGAGAT CTTTAAAACATTTAGTAGATGAAGCACATTGCCTTCGGTTGGATGCTATATACCATA ATATTACAGGAATATCTGTTGATGTCCACCAGCAAATTGTTTATTTCTCTGAAGGAA CTCTCATATGGGCGAAGAAGGCTGCCAACATGTCTGATGTATCTGACCTGAGAATTT TTTACAGAGGTTCAGGATTAATTTCTTCTATCTCCATAGATTGGCTTTATCAAAGAAT GTATTTCATCATGGATGAACTGGTATGTGTCTGTGATTTAGAGAACTGCTCAAACAT CGAGGAAATTACTCCACCCTCTATTAGTGCACCTCAAAAAATTGTGGCTGATTCATA CAATGGGTATGTCTTTTACCTCCTGAGAGATGGCATTTATAGAGCAGACCTTCCTGT ACCATCTGGCCGGTGTGCAGAAGCTGTGCGTATTGTGGAGAGTTGCACGTTAAAGG ACTTTGCAATCAAGCCACAAGCCAAGCGAATCATTTACTTCAATGACACTGCCCAAG TCTTCATGTCAACATTTCTGGATGGCTCTGCTTCCCATCTCATCCTACCTCGCATCCC CTTTGCTGATGTGAAAAGTTTTGCTTGTGAAAACAATGACTTTCTTGTCACAGATGG CAAGGTCATTTTCCAACAGGATGCTTTGTCTTTTAATGAATTCATCGTGGGATGTGA CCTGAGTCACATAGAAGAATTTGGGTTTGGTAACTTGGTCATCTTTGGCTCATCCTCC CAGCTGCACCCTCTGCCAGGCCGCCCGCAGGAGCTTTCGGTGCTGTTTGGCTCTCAC CAGGCTCTTGTTCAATGGAAGCCTCCTGCCCTTGCCATAGGAGCCAATGTCATCCTG ATCAGTGATATTATTGAACTCTTTGAATTAGGCCCTTCTGCCTGGCAGAACTGGACC TATGAGGTGAAAGTATCCACCCAAGACCCTCCTGAAGTCACTCATATTTTCTTGAAC ATAAGTGGAACCATGCTGAATGTACCTGAGCTGCAGAGTGCTATGAAATACAAGGT TTCTGTGAGAGCAAGTTCTCCAAAGAGGCCAGGCCCCTGGTCAGAGCCCTCAGTGG GTACTACCCTGGTGCCAGCTAGTGAACCACCATTTATCATGGCTGTGAAAGAAGATG GGCTTTGGAGTAAACCATTAAATAGCTTTGGCCCAGGAGAGTTCTTATCCTCTGATA TAGGAAATGTGTCAGACATGGATTGGTATAACAACAGCCTCTACTACAGTGACACG AAAGGCGACGTTTTTGTGTGGCTGCTGAATGGGACGGATATCTCAGAGAATTATCAC CTACCCAGCATTGCAGGAGCAGGGGCTTTAGCTTTTGAGTGGCTGGGTCACTTTCTC TACTGGGCTGGAAAGACATATGTGATACAAAGGCAGTCTGTGTTGACGGGACACAC AGACATTGTTACCCACGTGAAGCTATTGGTGAATGACATGGTGGTGGATTCAGTTGG TGGATATCTCTACTGGACCACACTCTATTCAGTGGAAAGCACCAGACTAAATGGGG AAAGTTCCCTTGTACTACAGACACAGCCTTGGTTTTCTGGGAAAAAGGTAATTGCTC TAACTTTAGACCTCAGTGATGGGCTCCTGTATTGGTTGGTTCAAGACAGTCAATGTA TTCACCTGTACACAGCTGTTCTTCGGGGACAGAGCACTGGGGATACCACCATCACAG AATTTGCAGCCTGGAGTACTTCTGAAATTTCCCAGAATGCACTGATGTACTATAGTG GTCGGCTGTTCTGGATCAATGGCTTTAGGATTATCACAACTCAAGAAATAGGTCAGA AAACCAGTGTCTCTGTTTTGGAACCAGCCAGATTTAATCAGTTCACAATTATTCAGA CATCCCTTAAGCCCCTGCCAGGGAACTTTTCCTTTACCCCTAAGGTTATTCCAGATTC TGTTCAAGAGTCTTCATTTAGGATTGAAGGAAATGCTTCAAGTTTTCAAATCCTGTG GAATGGTCCCCCTGCGGTAGACTGGGGTGTAGTTTTCTACAGTGTAGAATTTAGTGC TCATTCTAAGTTCTTGGCTAGTGAACAACACTCTTTACCTGTATTTACTGTGGAAGGA CTGGAACCTTATGCCTTATTTAATCTTTCTGTCACTCCTTATACCTACTGGGGAAAGG GCCCCAAAACATCTCTGTCACTTCGAGCACCTGAAACAGTTCCATCAGCACCAGAGA ACCCCAGAATATTTATATTACCAAGTGGAAAATGCTGCAACAAGAATGAAGTTGTG GTGGAATTTAGGTGGAACAAACCTAAGCATGAAAATGGGGTGTTAACAAAATTTGA AATTTTCTACAATATATCCAATCAAAGTATTACAAACAAAACATGTGAAGACTGGAT TGCTGTCAATGTCACTCCCTCAGTGATGTCTTTTCAACTTGAAGGCATGAGTCCCAG ATGCTTTATTGCCTTCCAGGTTAGGGCCTTTACATCTAAGGGGCCAGGACCATATGC TGACGTTGTAAAGTCTACAACATCAGAAATCAACCCATTTCCTCACCTCATAACTCT TCTTGGTAACAAGATAGTTTTTTTAGATATGGATCAAAATCAAGTTGTGTGGACGTT TTCAGCAGAAAGAGTTATCAGTGCCGTTTGCTACACAGCTGATAATGAGATGGGAT ATTATGCTGAAGGGGACTCACTCTTTCTTCTGCACTTGCACAATCGCTCTAGCTCTGA GCTTTTCCAAGATTCACTGGTTTTTGATATCACAGTTATTACAATTGACTGGATTTCA AGGCACCTCTACTTTGCACTGAAAGAATCACAAAATGGAATGCAAGTATTTGATGTT GATCTTGAACACAAGGTGAAATATCCCAGAGAGGTGAAGATTCACAATAGGAATTC AACAATAATTTCTTTTTCTGTATATCCTCTTTTAAGTCGCTTGTATTGGACAGAAGTT TCCAATTTTGGCTACCAGATGTTCTACTACAGTATTATCAGTCACACCTTGCACCGA ATTCTGCAACCCACAGCTACAAACCAACAAAACAAAAGGAATCAATGTTCTTGTAA TGTGACTGAATTTGAGTTAAGTGGAGCAATGGCTATTGATACCTCTAACCTAGAGAA ACCATTGATATACTTTGCCAAAGCACAAGAGATCTGGGCAATGGATCTGGAAGGCT GTCAGTGTTGGAGAGTTATCACAGTACCTGCTATGCTCGCAGGAAAAACCCTTGTTA GCTTAACTGTGGATGGAGATCTTATATACTGGATCATCACAGCAAAGGACAGCACA CAGATTTATCAGGCAAAGAAAGGAAATGGGGCCATCGTTTCCCAGGTGAAGGCCCT AAGGAGTAGGCATATCTTGGCTTACAGTTCAGTTATGCAGCCTTTTCCAGATAAAGC GTTTCTGTCTCTAGCTTCAGACACTGTGGAACCAACTATACTTAATGCCACTAACAC TAGCCTCACAATCAGATTACCTCTGGCCAAGACAAACCTCACATGGTATGGCATCAC CAGCCCTACTCCAACATACCTGGTTTATTATGCAGAAGTTAATGACAGGAAAAACA GCTCTGACTTGAAATATAGAATTCTGGAATTTCAGGACAGTATAGCTCTTATTGAAG ATTTACAACCATTTTCAACATACATGATACAGATAGCTGTAAAAAATTATTATTCAG ATCCTTTGGAACATTTACCACCAGGAAAAGAGATTTGGGGAAAAACTAAAAATGGA GTACCAGAGGCAGTGCAGCTCATTAATACAACTGTGCGGTCAGACACCAGCCTCATT ATATCTTGGAGAGAATCTCACAAGCCAAATGGACCTAAAGAATCAGTCCGTTATCA GTTGGCAATCTCACACCTGGCCCTAATTCCTGAAACTCCTCTAAGACAAAGTGAATT TCCAAATGGAAGGCTCACTCTCCTTGTTACTAGACTGTCTGGTGGAAATATTTATGT GTTAAAGGTTCTTGCCTGCCACTCTGAGGAAATGTGGTGTACAGAGAGTCATCCTGT CACTGTGGAAATGTTTAACACACCAGAGAAACCTTATTCCTTGGTTCCAGAGAACAC TAGTTTGCAATTTAATTGGAAGGCTCCATTGAATGTTAACCTCATCAGATTTTGGGTT GAGCTACAGAAGTGGAAATACAATGAGTTTTACCATGTTAAAACTTCATGCAGCCA AGGTCCTGCTTATGTCTGTAATATCACAAATCTACAACCTTATACTTCATATAATGTC AGAGTAGTGGTGGTTTATAAGACGGGAGAAAATAGCACCTCACTTCCAGAAAGCTT TAAGACAAAAGCTGGAGTCCCAAATAAACCAGGCATTCCCAAATTACTAGAAGGGA GTAAAAATTCAATACAGTGGGAGAAAGCTGAAGATAATGGATGTAGAATTACATAC TATATCCTTGAGATAAGAAAGAGCACTTCAAATAATTTACAGAACCAGAATTTAAG GTGGAAGATGACATTTAATGGATCCTGCAGTAGTGTTTGCACATGGAAGTCCAAAA ACCTGAAAGGAATATTTCAGTTCAGAGTAGTAGCTGCAAATAATCTAGGGTTTGGTG AATATAGTGGAATCAGTGAGAATATTATATTAGTTGGAGATGATTTTTGGATACCAG AAACAAGTTTCATACTTACTATTATAGTTGGAATATTTCTGGTTGTTACAATCCCACT GACCTTTGTCTGGCATAGAAGATTAAAGAATCAAAAAAGTGCCAAGGAAGGGGTGA CAGTGCTTATAAACGAAGACAAAGAGTTGGCTGAGCTGCGAGGTCTGGCAGCCGGA GTAGGCCTGGCTAATGCCTGCTATGCAATACATACTCTTCCAACCCAAGAGGAGATT GAAAATCTTCCTGCCTTCCCTCGGGAAAAACTGACTCTGCGTCTCTTGCTGGGAAGT GGAGCCTTTGGAGAAGTGTATGAAGGAACAGCAGTGGACATCTTAGGAGTTGGAAG TGGAGAAATCAAAGTAGCAGTGAAGACTTTGAAGAAGGGTTCCACAGACCAGGAG AAGATTGAATTCCTGAAGGAGGCACATCTGATGAGCAAATTTAATCATCCCAACATT CTGAAGCAGCTTGGAGTTTGTCTGCTGAATGAACCCCAATACATTATCCTGGAACTG ATGGAGGGAGGAGACCTTCTTACTTATTTGCGTAAAGCCCGGATGGCAACGTTTTAT GGTCCTTTACTCACCTTGGTTGACCTTGTAGACCTGTGTGTAGATATTTCAAAAGGCT GTGTCTACTTGGAACGGATGCATTTCATTCACAGGGATCTGGCAGCTAGAAATTGCC TTGTTTCCGTGAAAGACTATACCAGTCCACGGATAGTGAAGATTGGAGACTTTGGAC TCGCCAGAGACATCTATAAAAATGATTACTATAGAAAGAGAGGGGAAGGCCTGCTC CCAGTTCGGTGGATGGCTCCAGAAAGTTTGATGGATGGAATCTTCACTACTCAATCT GATGTATGGTCTTTTGGAATTCTGATTTGGGAGATTTTAACTCTTGGTCATCAGCCTT ATCCAGCTCATTCCAACCTTGATGTGTTAAACTATGTGCAAACAGGAGGGAGACTGG AGCCACCAAGAAATTGTCCTGATGATCTGTGGAATTTAATGACCCAGTGCTGGGCTC AAGAACCCGACCAAAGACCTACTTTTCATAGAATTCAGGACCAACTTCAGTTATTCA GAAATTTTTTCTTAAATAGCATTTATAAGTCCAGAGATGAAGCAAACAACAGTGGA GTCATAAATGAAAGCTTTGAAGGTGAAGATGGCGATGTGATTTGTTTGAATTCAGAT GACATTATGCCAGTTGCTTTAATGGAAACGAAGAACCGAGAAGGGTTAAACTATAT GGTACTTGCTACAGAATGTGGCCAAGGTGAAGAAAAGTCTGAGGGTCCTCTAGGCT CCCAGGAATCTGAATCTTGTGGTCTGAGGAAAGAAGAGAAGGAACCACATGCAGAC AAAGATTTCTGCCAAGAAAAACAAGTGGCTTACTGCCCTTCTGGCAAGCCTGAAGG CCTGAACTATGCCTGTCTCACTCACAGTGGATATGGAGATGGGTCTGATTAA (SEQ ID NO:348). In some embodiments, an RNA mutation results in a translocation, rearrangement, or fusion gene at the ROS locus. In some embodiments, an RNA mutation results in a fusion between the ROS gene and a gene selected from the group consisting of CD47, and SLC34A2. For example, in some embodiments, an RNA mutation in an ROS gene encodes or results in a C6;R32 or C6;R34 CD74-ROS1 fusion protein, or an S4;R32 or S4;R34 SLC34A2-ROS1 fusion protein, an SD2;R32 fusion protein.

In some embodiments, a primer pair for amplifying the locus of an ROS mutation (e.g., encoding or resulting in a C6;R32 or C6;R34 CD74-ROS1 fusion protein) comprises the sequence (e.g., that hybridizes with a CD74-specific locus of the fusion gene) GGAGTGCCATCGCTGTTTGAAATGAGCAGGCACT (SEQ ID NO:19) and another sequence (e.g., that hybridizes with an ROS-specific locus of the fusion gene) selected from the group consisting of AATTCAATACATACTATCAGCTTTCTCCCACTGTATTGAA (SEQ ID NO:39; for exon 32 fusions) and AATATTTCTGGTACGAGTGGGATTGTAACAACCAGAAATA (SEQ ID NO:40; for exon 34 fusions). In some embodiments, a primer pair for amplifying the locus of an ROS mutation (e.g., encoding or resulting in an S4;R32 or S4;R34 SLC34A2-ROS1 fusion protein) comprises the sequence (e.g., that hybridizes with an SLC34A2-specific locus of the fusion gene) TACAGCCCTGGATATTCTTAGTAGCGC (SEQ ID NO:20) and another sequence (e.g., that hybridizes with an ROS-specific locus of the fusion gene) selected from the group consisting of AATTCAATACATACTATCAGCTTTCTCCCACTGTATTGAA (SEQ ID NO:39; for exon 32 fusions) and AATATTTCTGGTACGAGTGGGATTGTAACAACCAGAAATA (SEQ ID NO:40; for exon 34 fusions).

In some embodiments, the methods of the present disclosure include amplifying the loci of one or more mutations (e.g., RNA mutations) in a RET gene. RET encodes the c-RET proto-oncogene, a receptor tyrosine kinase frequently mutated in human cancers, also known as PTC, RET51, RET9, CDHF12, CDHR16, HSCR1, MEN2A, MEN2B. MTC1, and RET-ELE1. In some embodiments, the RET gene is a human RET gene. In some embodiments, a human RET gene refers to the gene described by NCBI Entrez Gene ID No. 5979, including mutants and variants thereof. In other embodiments, the RET gene is from one of the following organisms: mouse (see, e.g., NCBI Entrez Gene ID No. 196713), rat (see, e.g., NCBI Entrez Gene ID No. 24716), fish (see, e.g., NCBI Entrez Gene ID No. 30512), cattle (see, e.g., NCBI Entrez Gene ID No. 515924), dog (see, e.g., NCBI Entrez Gene ID No. 403494), or chimpanzee (see, e.g., NCBI Entrez Gene ID No. 100612888).

A variety of RET mutations associated with cancer are known and may be suitably detected by the methods described herein; see, e.g., Riely, G. 2012. RET in Lung Cancer. My Cancer Genome at www.mycancergenome.org/content/disease/lung-cancer/ret/ (Updated December 13). Certain point mutations destabilize RET dimerization and result in constitutive activation of RET. RET gene fusions are found in 1-2% of adenocarcinoma type NSCLC and are generally mutually exclusive of mutations in EGFR, KRAS, ALK, and ROS1. Patients with RET rearrangements in NSCLC tend to be younger (≤60) and lack smoking history. Specific RET mutations, such as V804, may be responsible for insensitivity to TKIs such as vandetanib, motesanib, and cabozantinib, while retaining sensitivity to others, such as sunitinib and ponatinib. FDA approved drugs sensitive to RET include regorafenib, lenvatinib, ponatinib, cabozantinib, sorafenib, sunitinib, and vandetanib. Small molecule inhibitors targeting RET or downstream effectors RAF or MEK are under development for their efficacy in RET altered carcinoma. In some embodiments described herein, a RET mutation is named based on the resulting amino acid substitution/deletion/frameshift/translocation according to a human RET protein, e.g., as set forth in MAKATSGAAGLRLLLLLLLPLLGKVALGLYFSRDAYWEKLYVDQAAGTPLLYVHALR DAPEEVPSFRLGQHLYGTYRTRLHENNWICIQEDTGLLYLNRSLDHSSWEKLSVRNRGF PLLTVYLKVFLSPTSLREGECQWPGCARVYFSFFNTSFPACSSLKPRELCFPETRPSFRIRE NRPPGTFHQFRLLPVQFLCPNISVAYRLLEGEGLPFRCAPDSLEVSTRWALDREQREKYE LVAVCTVHAGAREEVVMVPFPVTVYDEDDSAPTFPAGVDTASAVVEFKRKEDTVVATL RVFDADVVPASGELVRRYTSTLLPGDTWAQQTFRVEHWPNETSVQANGSFVRATVHD YRLVLNRNLSISENRTMQLAVLVNDSDFQGPGAGVLLLHFNVSVLPVSLHLPSTYSLSVS RRARRFAQIGKVCVENCQAFSGINVQYKLHSSGANCSTLGVVTSAEDTSGILFVNDTKA LRRPKCAELHYMVVATDQQTSRQAQAQLLVTVEGSYVAEEAGCPLSCAVSKRRLECEE CGGLGSPTGRCEWRQGDGKGITRNFSTCSPSTKTCPDGHCDVVETQDINICPQDCLRGSI VGGHEPGEPRGIKAGYGTCNCFPEEEKCFCEPEDIQDPLCDELCRTVIAAAVLFSFIVSVL LSAFCIHCYHKFAHKPPISSAEMTFRRPAQAFPVSYSSSGARRPSLDSMENQVSVDAFKIL EDPKWEFPRKNLVLGKTLGEGEFGKVVKATAFHLKGRAGYTTVAVKMLKENASPSEL RDLLSEFNVLKQVNHPHVIKLYGACSQDGPLLLIVEYAKYGSLRGFLRESRKVGPGYLG SGGSRNSSSLDHPDERALTMGDLISFAWQISQGMQYLAEMKLVHRDLAARNILVAEGR KMKISDFGLSRDVYEEDSYVKRSQGRIPVKWMAIESLFDHIYTTQSDVWSFGVLLWEIV TLGGNPYPGIPPERLFNLLKTGHRMERPDNCSEEMYRLMLQCWKQEPDKRPVFADISKD LEKMMVKRRDYLDLAASTPSDSLIYDDGLSEEETPLVDCNNAPLPRALPSTWIENKLYG RISHAFTRF (SEQ ID NO:336). An exemplary human RET cDNA sequence is set forth in ATGGCGAAGGCGACGTCCGGTGCCGCGGGGCTGCGTCTGCTGTTGCTGCTGCTGCTG CCGCTGCTAGGCAAAGTGGCATTGGGCCTCTACTTCTCGAGGGATGCTTACTGGGAG AAGCTGTATGTGGACCAGGCAGCCGGCACGCCCTTGCTGTACGTCCATGCCCTGCGG GACGCCCCTGAGGAGGTGCCCAGCTTCCGCCTGGGCCAGCATCTCTACGGCACGTAC CGCACACGGCTGCATGAGAACAACTGGATCTGCATCCAGGAGGACACCGGCCTCCT CTACCTTAACCGGAGCCTGGACCATAGCTCCTGGGAGAAGCTCAGTGTCCGCAACC GCGGCTTTCCCCTGCTCACCGTCTACCTCAAGGTCTTCCTGTCACCCACATCCCTTCG TGAGGGCGAGTGCCAGTGGCCAGGCTGTGCCCGCGTATACTTCTCCTTCTTCAACAC CTCCTTTCCAGCCTGCAGCTCCCTCAAGCCCCGGGAGCTCTGCTTCCCAGAGACAAG GCCCTCCTTCCGCATTCGGGAGAACCGACCCCCAGGCACCTTCCACCAGTTCCGCCT GCTGCCTGTGCAGTTCTTGTGCCCCAACATCAGCGTGGCCTACAGGCTCCTGGAGGG TGAGGGTCTGCCCTTCCGCTGCGCCCCGGACAGCCTGGAGGTGAGCACGCGCTGGG CCCTGGACCGCGAGCAGCGGGAGAAGTACGAGCTGGTGGCCGTGTGCACCGTGCAC GCCGGCGCGCGCGAGGAGGTGGTGATGGTGCCCTTCCCGGTGACCGTGTACGACGA GGACGACTCGGCGCCCACCTTCCCCGCGGGCGTCGACACCGCCAGCGCCGTGGTGG AGTTCAAGCGGAAGGAGGACACCGTGGTGGCCACGCTGCGTGTCTTCGATGCAGAC GTGGTACCTGCATCAGGGGAGCTGGTGAGGCGGTACACAAGCACGCTGCTCCCCGG GGACACCTGGGCCCAGCAGACCTTCCGGGTGGAACACTGGCCCAACGAGACCTCGG TCCAGGCCAACGGCAGCTTCGTGCGGGCGACCGTACATGACTATAGGCTGGTTCTCA ACCGGAACCTCTCCATCTCGGAGAACCGCACCATGCAGCTGGCGGTGCTGGTCAAT GACTCAGACTTCCAGGGCCCAGGAGCGGGCGTCCTCTTGCTCCACTTCAACGTGTCG GTGCTGCCGGTCAGCCTGCACCTGCCCAGTACCTACTCCCTCTCCGTGAGCAGGAGG GCTCGCCGATTTGCCCAGATCGGGAAAGTCTGTGTGGAAAACTGCCAGGCATTCAGT GGCATCAACGTCCAGTACAAGCTGCATTCCTCTGGTGCCAACTGCAGCACGCTAGGG GTGGTCACCTCAGCCGAGGACACCTCGGGGATCCTGTTTGTGAATGACACCAAGGC CCTGCGGCGGCCCAAGTGTGCCGAACTTCACTACATGGTGGTGGCCACCGACCAGC AGACCTCTAGGCAGGCCCAGGCCCAGCTGCTTGTAACAGTGGAGGGGTCATATGTG GCCGAGGAGGCGGGCTGCCCCCTGTCCTGTGCAGTCAGCAAGAGACGGCTGGAGTG TGAGGAGTGTGGCGGCCTGGGCTCCCCAACAGGCAGGTGTGAGTGGAGGCAAGGAG ATGGCAAAGGGATCACCAGGAACTTCTCCACCTGCTCTCCCAGCACCAAGACCTGCC CCGACGGCCACTGCGATGTTGTGGAGACCCAAGACATCAACATTTGCCCTCAGGACT GCCTCCGGGGCAGCATTGTTGGGGGACACGAGCCTGGGGAGCCCCGGGGGATTAAA GCTGGCTATGGCACCTGCAACTGCTTCCCTGAGGAGGAGAAGTGCTTCTGCGAGCCC GAAGACATCCAGGATCCACTGTGCGACGAGCTGTGCCGCACGGTGATCGCAGCCGC TGTCCTCTTCTCCTTCATCGTCTCGGTGCTGCTGTCTGCCTTCTGCATCCACTGCTACC ACAAGTTTGCCCACAAGCCACCCATCTCCTCAGCTGAGATGACCTTCCGGAGGCCCG CCCAGGCCTTCCCGGTCAGCTACTCCTCTTCCGGTGCCCGCCGGCCCTCGCTGGACT CCATGGAGAACCAGGTCTCCGTGGATGCCTTCAAGATCCTGGAGGATCCAAAGTGG GAATTCCCTCGGAAGAACTTGGTTCTTGGAAAAACTCTAGGAGAAGGCGAATTTGG AAAAGTGGTCAAGGCAACGGCCTTCCATCTGAAAGGCAGAGCAGGGTACACCACGG TGGCCGTGAAGATGCTGAAAGAGAACGCCTCCCCGAGTGAGCTGCGAGACCTGCTG TCAGAGTTCAACGTCCTGAAGCAGGTCAACCACCCACATGTCATCAAATTGTATGGG GCCTGCAGCCAGGATGGCCCGCTCCTCCTCATCGTGGAGTACGCCAAATACGGCTCC CTGCGGGGCTTCCTCCGCGAGAGCCGCAAAGTGGGGCCTGGCTACCTGGGCAGTGG AGGCAGCCGCAACTCCAGCTCCCTGGACCACCCGGATGAGCGGGCCCTCACCATGG GCGACCTCATCTCATTTGCCTGGCAGATCTCACAGGGGATGCAGTATCTGGCCGAGA TGAAGCTCGTTCATCGGGACTTGGCAGCCAGAAACATCCTGGTAGCTGAGGGGCGG AAGATGAAGATTTCGGATTTCGGCTTGTCCCGAGATGTTTATGAAGAGGATTCCTAC GTGAAGAGGAGCCAGGGTCGGATTCCAGTTAAATGGATGGCAATTGAATCCCTTTTT GATCATATCTACACCACGCAAAGTGATGTATGGTCTTTTGGTGTCCTGCTGTGGGAG ATCGTGACCCTAGGGGGAAACCCCTATCCTGGGATTCCTCCTGAGCGGCTCTTCAAC CTTCTGAAGACCGGCCACCGGATGGAGAGGCCAGACAACTGCAGCGAGGAGATGTA CCGCCTGATGCTGCAATGCTGGAAGCAGGAGCCGGACAAAAGGCCGGTGTTTGCGG ACATCAGCAAAGACCTGGAGAAGATGATGGTTAAGAGGAGAGACTACTTGGACCTT GCGGCGTCCACTCCATCTGACTCCCTGATTTATGACGACGGCCTCTCAGAGGAGGAG ACACCGCTGGTGGACTGTAATAATGCCCCCCTCCCTCGAGCCCTCCCTTCCACATGG ATTGAAAACAAACTCTATGGTAGAATTTCCCATGCATTTACTAGATTCTAG (SEQ ID NO:349). In some embodiments, an RNA mutation results in a translocation, rearrangement, or fusion gene at the RET locus. In some embodiments, an RNA mutation results in a fusion between the RET gene and a gene selected from the group consisting of KIF5B, and CCDC6. For example, in some embodiments, an RNA mutation in a RET gene encodes or results in a K15;R11, K15;R12, K16;R12, K22;R11, or an K23;R12 KIF5B:RET fusion protein.

In some embodiments, a primer pair for amplifying the locus of a RET mutation (e.g., encoding or resulting in a K15;R11, K15;R12, K16;R12, K22;R12, or K23;R12KIF5B:RET fusion gene) comprises the sequences TTTCTGGTGCTATGAGGAAATGACCAACCACCAGA (SEQ ID NO:23) and GTGATCGCACAGTAGGACAGCGGCTGCGATC (SEQ ID NO:26) (e.g., for a K15;R11 KIF5B:RET fusion gene); the sequences TTTCTGGTGCTATGAGGAAATGACCAACCACCAGA (SEQ ID NO:23) and GTGATCGCACAGTAGGACAGCGGCTGCGATC (SEQ ID NO:27) (e.g., for a K15;R12 KIF5B:RET fusion gene), the sequences AAGGAGTTAGCAGCATGTCAGC (SEQ ID NO:519) and GTGATCGCACAGTAGGACAGCGGCTGCGATC (SEQ ID NO:27) (e.g., for a K16;R12 KIF5B:RET fusion gene), the sequences AACTTCAGACTTTACACAACCTGC (SEQ ID NO:520) and GTGATCGCACAGTAGGACAGCGGCTGCGATC (SEQ ID NO:27) (e.g., for a K22;R12 KIF5B:RET fusion gene), or the sequences ATTGATTCTGATGACACCGGA (SEQ ID NO:521) and GTGATCGCACAGTAGGACAGCGGCTGCGATC (SEQ ID NO:27) (e.g., for a K23;R12 KIF5B:RET fusion gene).

In some embodiments, the methods of the present disclosure include amplifying the loci of one or more mutations (e.g., RNA mutations) in an NTRK1 gene. NTRK1 encodes the tropomyosin receptor kinase A (TrkA, a receptor tyrosine kinase frequently mutated in human cancers, also known as the high affinity nerve growth factor receptor, neurotrophic tyrosine kinase receptor type 1, TRK1-transforming tyrosine kinase protein, MTC, TRK, TRKA, Trk-A, and p140-TrkA. In some embodiments, the NTRK1 gene is a human NTRK1 gene. In some embodiments, a human NTRK1 gene refers to the gene described by NCBI Entrez Gene ID No. 4914, including mutants and variants thereof. In other embodiments, the NTRK1 gene is from one of the following organisms: mouse (see, e.g., NCBI Entrez Gene ID No. 18211), rat (see, e.g., NCBI Entrez Gene ID No. 59109), fish (see, e.g., NCBI Entrez Gene ID No. 30546), cattle (see, e.g., NCBI Entrez Gene ID No. 353111), chicken (see, e.g., NCBI Entrez Gene ID No. 396337), or chimpanzee (see, e.g., NCBI Entrez Gene ID No. 457408).

A variety of NTRK1 mutations associated with cancer are known and may be suitably detected by the methods described herein; see, e.g., Loyly, C., R. Doebele. 2014. NTRK1 (TRKA) in Lung Cancer. My Cancer Genome at www.mycancergenome.org/content/disease/lung-cancer/ntrk1/ (Updated May 23). In some embodiments described herein, an NTRK1 mutation is named based on the resulting amino acid substitution/deletion/frameshift/translocation according to a human NTRK1 protein, e.g., as set forth in MKEAALICLAPSVPPILTVKSWDTMQLRAARSRCTNLLAASYIENQQHLQHLELRDLRG LGELRNLTIVKSGLRFVAPDAFHFTPRLSRLNLSFNALESLSWKTVQGLSLQELVLSGNP LHCSCALRWLQRWEEEGLGGVPEQKLQCHGQGPLAHMPNASCGVPTLKVQVPNASVD VGDDVLLRCQVEGRGLEQAGWILTELEQSATVMKSGGLPSLGLTLANVTSDLNRKNVT CWAENDVGRAEVSVQVNVSFPASVQLHTAVEMHHWCIPFSVDGQPAPSLRWLFNGSV LNETSFIFTEFLEPAANETVRHGCLRLNQPTHVNNGNYTLLAANPFGQASASIMAAFMD NPFEFNPEDPIPDTNSTSGDPVEKKDETPFGVSVAVGLAVFACLFLSTLLLVLNKCGRRN KFGINRPAVLAPEDGLAMSLHFMTLGGSSLSPTEGKGSGLQGHIIENPQYFSDACVHHIK RRDIVLKWELGEGAFGKVFLAECHNLLPEQDKMLVAVKALKEASESARQDFQREAELL TMLQHQHIVRFFGVCTEGRPLLMVFEYMRHGDLNRFLRSHGPDAKLLAGGEDVAPGPL GLGQLLAVASQVAAGMVYLAGLHFVHRDLATRNCLVGQGLVVKIGDFGMSRDIYSTD YYRVGGRTMLPIRWMPPESILYRKFTTESDVWSFGVVLWEIFTYGKQPWYQLSNTEAID CITQGRELERPRACPPEVYAIMRGCWQREPQQRHSIKDVHARLQALAQAPPVYLDVLG (SEQ ID NO:337). An exemplary human NTRK1 cDNA sequence is set forth in ATGAAGGAGGCCGCCCTCATCTGCCTGGCACCCTCTGTACCCCCGATCTTGACGGTG AAGTCCTGGGACACCATGCAGTTGCGGGCTGCTAGATCTCGGTGCACAAACTTGTTG GCAGCAAGCTACATCGAGAACCAGCAGCATCTGCAGCATCTGGAGCTCCGTGATCT GAGGGGCCTGGGGGAGCTGAGAAACCTCACCATCGTGAAGAGTGGTCTCCGTTTCG TGGCGCCAGATGCCTTCCATTTCACTCCTCGGCTCAGTCGCCTGAATCTCTCCTTCAA CGCTCTGGAGTCTCTCTCCTGGAAAACTGTGCAGGGCCTCTCCTTACAGGAACTGGT CCTGTCGGGGAACCCTCTGCACTGTTCTTGTGCCCTGCGCTGGCTACAGCGCTGGGA GGAGGAGGGACTGGGCGGAGTGCCTGAACAGAAGCTGCAGTGTCATGGGCAAGGG CCCCTGGCCCACATGCCCAATGCCAGCTGTGGTGTGCCCACGCTGAAGGTCCAGGTG CCCAATGCCTCGGTGGATGTGGGGGACGACGTGCTGCTGCGGTGCCAGGTGGAGGG GCGGGGCCTGGAGCAGGCCGGCTGGATCCTCACAGAGCTGGAGCAGTCAGCCACGG TGATGAAATCTGGGGGTCTGCCATCCCTGGGGCTGACCCTGGCCAATGTCACCAGTG ACCTCAACAGGAAGAACGTGACGTGCTGGGCAGAGAACGATGTGGGCCGGGCAGA GGTCTCTGTTCAGGTCAACGTCTCCTTCCCGGCCAGTGTGCAGCTGCACACGGCGGT GGAGATGCACCACTGGTGCATCCCCTTCTCTGTGGATGGGCAGCCGGCACCGTCTCT GCGCTGGCTCTTCAATGGCTCCGTGCTCAATGAGACCAGCTTCATCTTCACTGAGTT CCTGGAGCCGGCAGCCAATGAGACCGTGCGGCACGGGTGTCTGCGCCTCAACCAGC CCACCCACGTCAACAACGGCAACTACACGCTGCTGGCTGCCAACCCCTTCGGCCAG GCCTCCGCCTCCATCATGGCTGCCTTCATGGACAACCCTTTCGAGTTCAACCCCGAG GACCCCATCCCTGACACTAACAGCACATCTGGAGACCCGGTGGAGAAGAAGGACGA AACACCTTTTGGGGTCTCGGTGGCTGTGGGCCTGGCCGTCTTTGCCTGCCTCTTCCTT TCTACGCTGCTCCTTGTGCTCAACAAATGTGGACGGAGAAACAAGTTTGGGATCAAC CGCCCGGCTGTGCTGGCTCCAGAGGATGGGCTGGCCATGTCCCTGCATTTCATGACA TTGGGTGGCAGCTCCCTGTCCCCCACCGAGGGCAAAGGCTCTGGGCTCCAAGGCCA CATCATCGAGAACCCACAATACTTCAGTGATGCCTGTGTTCACCACATCAAGCGCCG GGACATCGTGCTCAAGTGGGAGCTGGGGGAGGGCGCCTTTGGGAAGGTCTTCCTTG CTGAGTGCCACAACCTCCTGCCTGAGCAGGACAAGATGCTGGTGGCTGTCAAGGCA CTGAAGGAGGCGTCCGAGAGTGCTCGGCAGGACTTCCAGCGTGAGGCTGAGCTGCT CACCATGCTGCAGCACCAGCACATCGTGCGCTTCTTCGGCGTCTGCACCGAGGGCCG CCCCCTGCTCATGGTCTTTGAGTATATGCGGCACGGGGACCTCAACCGCTTCCTCCG ATCCCATGGACCTGATGCCAAGCTGCTGGCTGGTGGGGAGGATGTGGCTCCAGGCC CCCTGGGTCTGGGGCAGCTGCTGGCCGTGGCTAGCCAGGTCGCTGCGGGGATGGTG TACCTGGCGGGTCTGCATTTTGTGCACCGGGACCTGGCCACACGCAACTGTCTAGTG GGCCAGGGACTGGTGGTCAAGATTGGTGATTTTGGCATGAGCAGGGATATCTACAG CACCGACTATTACCGTGTGGGAGGCCGCACCATGCTGCCCATTCGCTGGATGCCGCC CGAGAGCATCCTGTACCGTAAGTTCACCACCGAGAGCGACGTGTGGAGCTTCGGCG TGGTGCTCTGGGAGATCTTCACCTACGGCAAGCAGCCCTGGTACCAGCTCTCCAACA CGGAGGCAATCGACTGCATCACGCAGGGACGTGAGTTGGAGCGGCCACGTGCCTGC CCACCAGAGGTCTACGCCATCATGCGGGGCTGCTGGCAGCGGGAGCCCCAGCAACG CCACAGCATCAAGGATGTGCACGCCCGGCTGCAAGCCCTGGCCCAGGCACCTCCTG TCTACCTGGATGTCCTGGGCTAG (SEQ ID NO:350). In some embodiments, an RNA mutation results in a translocation, rearrangement, or fusion gene at the NTRK1 locus. In some embodiments, an RNA mutation results in a fusion between the NTRK1 and CD74 genes. For example, in some embodiments, an RNA mutation in an NTRK1 gene encodes or results in a C8;N12 CD74:NTRK1 fusion protein.

In some embodiments, a primer pair for amplifying the locus of an NTRK1 mutation (e.g., encoding or resulting in a C8;N12 CD74:NTRK1 fusion protein) comprises the sequence (e.g., that hybridizes with a CD74-specific locus of the fusion gene) AGAAGACGTGACAGGAACTGGAGGACCCGTCTT (SEQ ID NO:30) and the sequence (e.g., that hybridizes with an NTRK1-specific locus of the fusion gene) GGACGAAAATCCAGACCCCAAAAGGTGTTTCGT (SEQ ID NO:32).

In some embodiments, the methods of the present disclosure include amplifying the loci of one or more mutations (e.g., RNA mutations) in a cMET gene. cMET encodes the tyrosine protein kinase Met frequently mutated in human cancers, also known as the hepatocyte growth factor receptor (HGFR), AUTS9, RCCP2, DFNB97, and OSFD. In some embodiments, the cMET gene is a human cMET gene. In some embodiments, a human cMET gene refers to the gene described by NCBI Entrez Gene ID No. 4233, including mutants and variants thereof. In other embodiments, the cMET gene is from one of the following organisms: mouse (see, e.g., NCBI Entrez Gene ID No. 17295), rat (see, e.g., NCBI Entrez Gene ID No. 24553), fish (see, e.g., NCBI Entrez Gene ID No. 100150664), cattle (see, e.g., NCBI Entrez Gene ID No. 280855), chicken (see, e.g., NCBI Entrez Gene ID No. 396134), or chimpanzee (see, e.g., NCBI Entrez Gene ID No. 463671).

A variety of cMET mutations associated with cancer are known and may be suitably detected by the methods described herein; see, e.g., Loyly, C., P. Paik. 2017. MET Exon 14 Skipping Mutations in Lung Cancer. My Cancer Genome at www.mycancergenome.org/content/disease/lung-cancer/met/343 (Updated June 15). Occasional mutations in MET have been noted across the extracellular domain (residues 52-496), the juxtamembrane domain (residues 956-1093), and the tyrosine kinase domain (residues 1096-1355). The most frequently observed validated gain of function mutation in MET is Y1253D. Translocations of MET gene are rare, but have been noted in lung adenocarcinoma. Skipping of exon 14 and disruption of juxtamembrane domain activates MET in NSCLC and is sensitive to MET inhibitors. MET is amplified in 6% of sqNSCLC and copy number gain is seen in 2.7% of lung cancer. MET overexpression and amplification is generally associated with poor prognosis. Testing for MET mutations can be useful in determining a patient's sensitivity to various tyrosine kinase inhibitors. FDA approved drugs sensitive to MET include cabozantinib and crizotinib. In some embodiments described herein, an cMET mutation is named based on the resulting amino acid substitution/deletion/frameshift/translocation according to a human cMET protein, e.g., as set forth in MKAPAVLAPGILVLLFTLVQRSNGECKEALAKSEMNVNMKYQLPNFTAETPIQNVILHE HHIFLGATNYIYVLNEEDLQKVAEYKTGPVLEHPDCFPCQDCSSKANL SGGVWKDNIN MALVVDTYYDDQLISCGSVNRGTCQRHVFPHNHTADIQSEVHCIFSPQIEEPSQCPDCVV SALGAKVLSSVKDRFINFFVGNTINSSYFPDHPLHSISVRRLKETKDGFMFLTDQSYIDVL PEFRDSYPIKYVHAFESNNFIYFLTVQRETLDAQTFHTRIIRFCSINSGLHSYMEMPLECIL TEKRKKRSTKKEVFNILQAAYVSKPGAQLARQIGASLNDDILFGVFAQSKPDSAEPMDR SAMCAFPIKYVNDFFNKIVNKNNVRCLQHFYGPNHEHCFNRTLLRNSSGCEARRDEYRT EFTTALQRVDLFMGQFSEVLLTSISTFIKGDLTIANLGTSEGRFMQVVVSRSGPSTPHVNF LLDSHPVSPEVIVEHTLNQNGYTLVITGKKITKIPLNGLGCRHFQSCSQCLSAPPFVQCG WCHDKCVRSEECLSGTWTQQICLPAIYKVFPNSAPLEGGTRLTICGWDFGFRRNNKFDL KKTRVLLGNESCTLTLSESTMNTLKCTVGPAMNKHFNMSIIISNGHGTTQYSTFSYVDPV ITSISPKYGPMAGGTLLTLTGNYLNSGNSRHISIGGKTCTLKSVSNSILECYTPAQTISTEF AVKLKIDLANRETSIFSYREDPIVYEIHPTKSFISGGSTITGVGKNLNSVSVPRMVINVHEA GRNFTVACQHRSNSEIICCTTPSLQQLNLQLPLKTKAFFMLDGILSKYFDLIYVHNPVFKP FEKPVMISMGNENVLEIKGNDIDPEAVKGEVLKVGNKSCENIHLHSEAVLCTVPNDLLK LNSELNIEWKQAISSTVLGKVIVQPDQNFTGLIAGVVSISTALLLLLGFFLWLKKRKQIKD LGSELVRYDARVHTPHLDRLVSARSVSPTTEMVSNESVDYRATFPEDQFPNSSQNGSCR QVQYPLTDMSPILTSGDSDISSPLLQNTVHIDLSALNPELVQAVQHVVIGPSSLIVHFNEVI GRGHFGCVYHGTLLDNDGKKIHCAVKSLNRITDIGEVSQFLTEGIIMKDFSHPNVLSLLG ICLRSEGSPLVVLPYMKHGDLRNFIRNETHNPTVKDLIGFGLQVAKGMKYLASKKFVHR DLAARNCMLDEKFTVKVADFGLARDMYDKEYYSVHNKTGAKLPVKWMALESLQTQK FTTKSDVWSFGVLLWELMTRGAPPYPDVNTFDITVYLLQGRRLLQPEYCPDPLYEVML KCWHPKAEMRPSFSELVSRISAIFSTFIGEHYVHVNATYVNVKCVAPYPSLLSSEDNADD EVDTRPASFWETS (SEQ ID NO:338). An exemplary human cMET cDNA sequence is set forth in ATGCCCAAGAAGAAGCCGACGCCCATCCAGCTGAACCCGGCCCCCGACGGCTCTGC AGTTAACGGGACCAGCTCTGCGGAGACCAACTTGGAGGCCTTGCAGAAGAAGCTGG AGGAGCTAGAGCTTGATGAGCAGCAGCGAAAGCGCCTTGAGGCCTTTCTTACCCAG AAGCAGAAGGTGGGAGAACTGAAGGATGACGACTTTGAGAAGATCAGTGAGCTGG GGGCTGGCAATGGCGGTGTGGTGTTCAAGGTCTCCCACAAGCCTTCTGGCCTGGTCA TGGCCAGAAAGCTAATTCATCTGGAGATCAAACCCGCAATCCGGAACCAGATCATA AGGGAGCTGCAGGTTCTGCATGAGTGCAACTCTCCGTACATCGTGGGCTTCTATGGT GCGTTCTACAGCGATGGCGAGATCAGTATCTGCATGGAGCACATGGATGGAGGTTC TCTGGATCAAGTCCTGAAGAAAGCTGGAAGAATTCCTGAACAAATTTTAGGAAAAG TTAGCATTGCTGTAATAAAAGGCCTGACATATCTGAGGGAGAAGCACAAGATCATG CACAGAGATGTCAAGCCCTCCAACATCCTAGTCAACTCCCGTGGGGAGATCAAGCT CTGTGACTTTGGGGTCAGCGGGCAGCTCATCGACTCCATGGCCAACTCCTTCGTGGG CACAAGGTCCTACATGTCGCCAGAAAGACTCCAGGGGACTCATTACTCTGTGCAGTC AGACATCTGGAGCATGGGACTGTCTCTGGTAGAGATGGCGGTTGGGAGGTATCCCA TCCCTCCTCCAGATGCCAAGGAGCTGGAGCTGATGTTTGGGTGCCAGGTGGAAGGA GATGCGGCTGAGACCCCACCCAGGCCAAGGACCCCCGGGAGGCCCCTTAGCTCATA CGGAATGGACAGCCGACCTCCCATGGCAATTTTTGAGTTGTTGGATTACATAGTCAA CGAGCCTCCTCCAAAACTGCCCAGTGGAGTGTTCAGTCTGGAATTTCAAGATTTTGT GAATAAATGCTTAATAAAAAACCCCGCAGAGAGAGCAGATTTGAAGCAACTCATGG TTCATGCTTTTATCAAGAGATCTGATGCTGAGGAAGTGGATTTTGCAGGTTGGCTCT GCTCCACCATCGGCCTTAACCAGCCCAGCACACCAACCCATGCTGCTGGCGTCTAA (SEQ ID NO:351). In some embodiments, an RNA mutation results in skipping of one or more exons at the cMET locus and/or amplification of the cMET locus. In some embodiments, an RNA mutation results in exon 14 skipping at the cMET locus.

In some embodiments, a primer pair for amplifying the locus of a cMET mutation (e.g., encoding or resulting in skipping of cMET exon 14) comprises the sequences GAATTTCACAGGATTGATTGCTGGTGTTGTCTC (SEQ ID NO:28) and GACAGTATTTTGCAGTAATGGACTGGATATATCAGA (SEQ ID NO:29).

In some embodiments of any of the above embodiments, a first primer (e.g., for generating cDNA) comprises a 5′ modification or label, such as biotin.

Exemplary and non-limiting RNA mutations are provided in Table A2 below.

A multiplex assay of the present disclosure may include detecting two or more of the mutations described above in combination. For example, an assay may include detecting two or more of the DNA mutations described above and/or two or more of the RNA mutations described above in combination.

In some embodiments, the methods of the present disclosure further comprise the use of microcarriers with an identifier corresponding to a positive or negative control.

In some embodiments, the methods of the present disclosure comprise amplifying a positive control sequence from isolated RNA by reverse transcription-polymerase chain reaction (RT-PCR). The positive control RNA sequence can be any sequence that is likely to be present in all samples of a given type. e.g., a non-mutated or endogenous gene sequence from the organism from whence the sample is obtained. The positive control indicates that RNA (e.g., human RNA) is present in the sample at levels sufficient for detection. Like the mutated RNA sequences of interest, the positive control sequence is detected by generating cDNA specific for the positive control sequence from the isolated RNA (e.g., by using a first primer specific for the positive control sequence) and amplifying DNA specific for the positive control sequence by PCR using the cDNA specific for the positive control sequence. The amplified positive control gene sequence is hybridized with a probe specific for the positive control gene sequence (the probe specific for the positive control gene sequence is coupled to a microcarrier with an identifier corresponding to a positive control). The presence or absence of hybridization of the amplified positive control sequence with the probe specific for the positive control gene sequence is then detected (and the analog code of the microcarrier with the identifier corresponding to the positive control is also detected).

In some embodiments, as exemplified infra, the positive control RNA sequence comprises a sequence of a hypoxanthine phosphoribosyltransferase 1 (HPRT1) gene (e.g., a human HPRT1 gene), also known as HGPRT or HPRT. In some embodiments, a primer pair specific for the positive control RNA sequence comprises the sequences GGAAGATATAATTGACACTGGCAAAACA (SEQ ID NO:34) and ATTCATTATAGTCAAGGGCATATCC (SEQ ID NO:35).

Blocking Nucleic Acids

In some embodiments, the methods of the present disclosure include amplifying isolated DNA by PCR in the presence of one or more blocking nucleic acid(s) (e.g., a blocking nucleic acid corresponding to the wild-type version of each DNA mutation of interest). Advantageously, the blocking nucleic acid prevents amplification of the wild-type DNA locus, thus increasing the sensitivity of detecting the DNA mutation (cf. FIGS. 4 & 5). In some embodiments, the methods include amplifying isolated DNA by PCR in the presence of at least seven blocking nucleic acids, each of which hybridizes with the wild-type DNA locus corresponding with a DNA mutation in the KRAS, NRAS, PIK3CA, BRAF, EGFR, AKT1, MEK1, or HER2 gene (e.g., at least one blocking nucleic acid per gene).

In some embodiments, a blocking nucleic acid of the present disclosure comprises: a single-stranded oligonucleotide that hybridizes with the corresponding wild-type DNA locus, and a 3′ terminal moiety that blocks extension from the single-stranded oligonucleotide, thereby preventing amplification of the wild-type DNA locus. In some embodiments, the 3′ terminal moiety comprises one or more inverted deoxythymidines (invdTs). In certain embodiments, the 3′ terminal moiety comprises three consecutive inverted deoxythymidines.

In some embodiments, a blocking nucleic acid of the present disclosure comprises one or more modified nucleotides. Oligonucleotides comprising modified nucleotides in some or all sequence positions are contemplated and may have improved hybridization properties particularly advantageous for use as a blocking nucleic acid during PCR. For example, it is known that oligonucleotides partly or completely synthesized using locked nucleic acids (LNAs) possess greater thermal stability than corresponding oligonucleotides synthesized with only conventional nucleotides, thereby increasing the melting temperature of an oligo:LNA duplex and allowing for shorter sequences that retain stable hybridization during thermocycling. See Koshkin, A. A. et al. (1998) Tetrahedron 54:3607-30. A variety of modified nucleotides are known and include without limitation locked nucleic acids (LNAs), peptide nucleic acids (PNAs), hexose nucleic acids (HNAs), threose nucleic acids (TNAs), glycol nucleic acids (GNAs), and cyclohexenyl nucleic acids (CeNAs). For more detailed description of exemplary modified nucleotides, see, e.g., Schmidt, M. (2010) BioEssays 32:322-31.

In some embodiments, a blocking nucleic acid of the present disclosure hybridizes with a wild-type KRAS locus corresponding with the locus of one or more DNA mutations at G12 or G13 of KRAS, e.g., DNA mutation(s) encoding a G12D, G12V, or G12C mutated KRAS protein. In some embodiments, the blocking nucleic acid comprises the sequence TACGCCACCAGCT(invdT)ₙ, wherein n is 1, 2, or 3 (SEQ ID NO:281); TTGGAGCTGGTGGCGTA(invdT)ₙ, wherein n is 1, 2, or 3 (SEQ ID NO:282); GCTGGTGGCGTAGGCA(invdT)ₙ, wherein n is 1, 2, or 3 (SEQ ID NO: 283); GCTGGTGGCGTAGGC(invdT)ₙ, wherein n is 1, 2, or 3 (SEQ ID NO:284), or TTGGAGCTGGTGGCGT(invdT)ₙ, wherein n is 1, 2, or 3 (SEQ ID NO:285); with italicized nucleic acids representing locked nucleic acids. In some embodiments, the blocking nucleic acid comprises the sequence TACGCCACCAGCT(invdT)ₙ, wherein n is 1, 2, or 3 (SEQ ID NO:281); TTGGAGCTGGTGGCGTA(invdT)ₙ, wherein n is 1, 2, or 3 (SEQ ID NO:282); GCTGGTGGCGTAGGCA(invdT)ₙ, wherein n is 1, 2, or 3 (SEQ ID NO:283); GCTGGTGGCGTAGGC(invdT)ₙ, wherein n is 1, 2, or 3 (SEQ ID NO:284); or TTGGAGCTGGTGGCGT(invdT)ₙ, wherein n is 1, 2, or 3 (SEQ ID NO:285); with underlined nucleic acids representing locked nucleic acids. In certain embodiments, n is 3. In some embodiments, the blocking nucleic acid comprises a sequence of SEQ ID NOs:281-285 but optionally includes a different pattern or type of modified nucleotide(s). In some embodiments, the blocking nucleic acid comprises a sequence of SEQ ID NOs:281-285 but includes a different 3′ terminal moiety.

In some embodiments, a blocking nucleic acid of the present disclosure hybridizes with a wild-type NRAS locus corresponding with the locus of one or more DNA mutations at Q61 of NRAS, e.g., DNA mutation(s) encoding a Q61L mutated NRAS protein. In some embodiments, the blocking nucleic acid comprises the sequence CTCTTCTTGTCCAG(invdT)ₙ, wherein n is 1, 2, or 3 (SEQ ID NO:286); TCTTCTTGTCCAGCTGTATCC(invdT)ₙ, wherein n is 1, 2, or 3 (SEQ ID NO:287); TCTTGTCCAGCTGT(invdT)ₙ, wherein n is 1, 2, or 3 (SEQ ID NO:288); TCTTGTCCAGCTGTATC(invdT)ₙ, wherein n is 1, 2, or 3 (SEQ ID NO:289); or TCTTCTTGTCCAGCTG(invdT)ₙ, wherein n is 1, 2, or 3 (SEQ ID NO:290); with italicized nucleic acids representing locked nucleic acids. In some embodiments, the blocking nucleic acid comprises the sequence CTCTTCTTGTCCAG(invdT)ₙ, wherein n is 1, 2, or 3 (SEQ ID NO:286); TCTTCTTGTCCAGCTGTATCC(invdT)ₙ, wherein n is 1, 2, or 3 (SEQ ID NO:287); TCTTGTCCAGCTGT(invdT)ₙ, wherein n is 1, 2, or 3 (SEQ ID NO:288); TCTTGTCCAGCTGTATC(invdT)ₙ, wherein n is 1, 2, or 3 (SEQ ID NO:289); or TCTTCTTGTCCAGCTG(invdT)ₙ, wherein n is 1, 2, or 3 (SEQ ID NO:290); with underlined nucleic acids representing locked nucleic acids. In some embodiments of any of the above embodiments, n is 1, 2, 3, 4, 5, 6, 7, 8, 9, or 10. In certain embodiments, n is 3. In some embodiments, the blocking nucleic acid comprises a sequence of SEQ ID NOs:286-290 but optionally includes a different pattern or type of modified nucleotide(s). In some embodiments, the blocking nucleic acid comprises a sequence of SEQ ID NOs: 286-290 but includes a different 3′ terminal moiety.

In some embodiments, a blocking nucleic acid of the present disclosure hybridizes with a wild-type PIK3CA locus corresponding with the locus of one or more DNA mutations at E542 or E545 of PIK3CA, e.g., DNA mutation(s) encoding an E542K, or E545K mutated PIK3CA protein. In some embodiments, the blocking nucleic acid comprises the sequence CTGAAATCACTGAGCAGG(invdT)ₙ, wherein n is 1, 2, or 3 (SEQ ID NO:291); TCTCTGAAATCACTGAGCAGG(invdT)ₙ, wherein n is 1, 2, or 3 (SEQ ID NO:292); TCTCTGAAATCACTGAGCAGG(invdT)ₙ, wherein n is 1, 2, or 3 (SEQ ID NO:293); TCTCTGAAATCACTGAGCAGG(invdT)ₙ, wherein n is 1, 2, or 3 (SEQ ID NO:294); or TCTCTGAATTCACTGAGCAGG (invdT)ₙ, wherein n is 1, 2, or 3 (SEQ ID NO:295); with italicized nucleic acids representing locked nucleic acids. In some embodiments, the blocking nucleic acid comprises the sequence CTGAAATCACTGAGCAGG (invdT)ₙ, wherein n is 1, 2, or 3 (SEQ ID NO:291); TCTCTGAAATCACTGAGCAGG(invdT)ₙ, wherein n is 1, 2, or 3 (SEQ ID NO:292); TCTCTGAAATCACTGAGCAGG(invdT)ₙ, wherein n is 1, 2, or 3 (SEQ ID NO:293); TCTCTGAAATCACTGAGCAGG(invdT)ₙ, wherein n is 1, 2, or 3 (SEQ ID NO:294); or TCTCTGAATTCACTGAGCAGG(invdT)ₙ, wherein n is 1, 2, or 3 (SEQ ID NO:295); with underlined nucleic acids representing locked nucleic acids. In some embodiments, a blocking nucleic acid of the present disclosure hybridizes with a wild-type PIK3CA locus corresponding with the locus of one or more DNA mutations at H1047 of PIK3CA, e.g., DNA mutation(s) encoding an H1047R mutated PIK3CA protein. In some embodiments, the blocking nucleic acid comprises the sequence CACCATGATGTGCAT (invdT)ₙ, wherein n is 1, 2, or 3 (SEQ ID NO:296); CCACCATGATGTGCAT (invdT)ₙ, wherein n is 1, 2, or 3 (SEQ ID NO:297); CACCATGATGTGCAT (invdT)ₙ, wherein n is 1, 2, or 3 (SEQ ID NO:298); CCACCATGATGTGCATCA (invdT)ₙ, wherein n is 1, 2, or 3 (SEQ ID NO:299); or CATGATGTGCA (invdT)ₙ, wherein n is 1, 2, or 3 (SEQ ID NO:300); with italicized nucleic acids representing locked nucleic acids. In some embodiments, the blocking nucleic acid comprises the sequence CACCATGATGTGCAT (invdT)ₙ, wherein n is 1, 2, or 3 (SEQ ID NO:296); CCACCATGATGTGCAT (invdT)ₙ, wherein n is 1, 2, or 3 (SEQ ID NO:297); CACCATGATGTGCAT (invdT)ₙ, wherein n is 1, 2, or 3 (SEQ ID NO:298); CCACCATGATGTGCATCA (invdT)ₙ, wherein n is 1, 2, or 3 (SEQ ID NO:299); or CATGATGTGCA (invdT)ₙ, wherein n is 1, 2, or 3 (SEQ ID NO:300); with underlined nucleic acids representing locked nucleic acids. In some embodiments of any of the above embodiments, n is 1, 2, 3, 4, 5, 6, 7, 8, 9, or 10. In certain embodiments, n is 3. In some embodiments, the blocking nucleic acid comprises a sequence of SEQ ID NOs:291-300 but optionally includes a different pattern or type of modified nucleotide(s). In some embodiments, the blocking nucleic acid comprises a sequence of SEQ ID NOs: 291-300 but includes a different 3′ terminal moiety.

In some embodiments, a blocking nucleic acid of the present disclosure hybridizes with a wild-type BRAF locus corresponding with the locus of one or more DNA mutations at V600 of BRAF, e.g., DNA mutation(s) encoding a V600E mutated BRAF protein. In some embodiments, the blocking nucleic acid comprises the sequence GAGATTTCACTGTAGC (invdT)ₙ, wherein n is 1, 2, or 3 (SEQ ID NO:301); GAGATTTCACTGTAGC (invdT)ₙ, wherein n is 1, 2, or 3 (SEQ ID NO:302); GAGATTCACTGTAGC (invdT)ₙ, wherein n is 1, 2, or 3 (SEQ ID NO:303); GAGATTCACTGTAGC (invdT)ₙ, wherein n is 1, 2, or 3 (SEQ ID NO:304); or GAGATTTCACTGTAGC (invdT)ₙ, wherein n is 1, 2, or 3 (SEQ ID NO:305); with italicized nucleic acids representing locked nucleic acids. In some embodiments, the blocking nucleic acid comprises the sequence GAGATTTCACTGTAGC (invdT)ₙ, wherein n is 1, 2, or 3 (SEQ ID NO:301); GAGATTTCACTGTAGC (invdT)ₙ, wherein n is 1, 2, or 3 (SEQ ID NO:302); GAGATTTCACTGTAGC (invdT)ₙ, wherein n is 1, 2, or 3 (SEQ ID NO:303); GAGATTTCACTGTAGC (invdT)ₙ, wherein n is 1, 2, or 3 (SEQ ID NO:304); or GAGATTTCACTGTAGC (invdT)ₙ, wherein n is 1, 2, or 3 (SEQ ID NO:305); with underlined nucleic acids representing locked nucleic acids. In some embodiments of any of the above embodiments, n is 1, 2, 3, 4, 5, 6, 7, 8, 9, or 10. In certain embodiments, n is 3. In some embodiments, the blocking nucleic acid comprises a sequence of SEQ ID NOs:301-305 but optionally includes a different pattern or type of modified nucleotide(s). In some embodiments, the blocking nucleic acid comprises a sequence of SEQ ID NOs: 301-305 but includes a different 3′ terminal moiety.

In some embodiments, a blocking nucleic acid of the present disclosure hybridizes with a wild-type EGFR locus corresponding with the locus of one or more DNA mutations at G719 of EGFR, e.g., DNA mutation(s) encoding a G719A mutated EGFR protein. In some embodiments, the blocking nucleic acid comprises the sequence CGGAGCCCAGCACTTTGA (invdT)ₙ, wherein n is 1, 2, or 3 (SEQ ID NO:306); CGCACCGGAGCCCAGCACT (invdT)ₙ, wherein n is 1, 2, or 3 (SEQ ID NO:307); GAGCCCAGCAC (invdT)ₙ, wherein n is 1, 2, or 3 (SEQ ID NO:308); CGCACCGGAGCCCAGCAC (invdT)ₙ, wherein n is 1, 2, or 3 (SEQ ID NO:309); or CGCACCGGAGCCCAGCACTTA (invdT)ₙ, wherein n is 1, 2, or 3 (SEQ ID NO:310); with italicized nucleic acids representing locked nucleic acids. In some embodiments, the blocking nucleic acid comprises the sequence CGGAGCCCAGCACTTTGA (invdT)ₙ, wherein n is 1, 2, or 3 (SEQ ID NO:306); CGCACCGGAGCCCAGCACT (invdT)ₙ, wherein n is 1, 2, or 3 (SEQ ID NO:307); GAGCCCAGCAC (invdT)ₙ, wherein n is 1, 2, or 3 (SEQ ID NO:308); CGCACCGGAGCCCAGCAC (invdT)ₙ, wherein n is 1, 2, or 3 (SEQ ID NO:309); or CGCACCGGAGCCCAGCACTTA (invdT)ₙ, wherein n is 1, 2, or 3 (SEQ ID NO:310); with underlined nucleic acids representing locked nucleic acids. In some embodiments, a blocking nucleic acid of the present disclosure hybridizes with a wild-type EGFR locus corresponding with the locus of one or more DNA mutations at E746-A750 of EGFR, e.g., DNA mutation(s) encoding an E746_A750del mutated EGFR protein. In some embodiments, the blocking nucleic acid comprises the sequence CGGAGATGTTGCTTCTCTTAATTCC (invdT)ₙ, wherein n is 1, 2, or 3 (SEQ ID NO:311); CGGAGATGTTGCTTCTCT (invdT)ₙ, wherein n is 1, 2, or 3 (SEQ ID NO:312); GTTGCTTCTCTTAATTCC (invdT)ₙ, wherein n is 1, 2, or 3 (SEQ ID NO:313); ATGTTGCTTCTCT (invdT)ₙ, wherein n is 1, 2, or 3 (SEQ ID NO:314); or TTGCTTCTCTTA (invdT)ₙ, wherein n is 1, 2, or 3 (SEQ ID NO:315); with italicized nucleic acids representing locked nucleic acids. In some embodiments, the blocking nucleic acid comprises the sequence CGGAGATGTTGCTTCTCTTAATTCC(invdT)ₙ, wherein n is 1, 2, or 3 (SEQ ID NO:311); CGGAGATGTTGCTTCTCT(invdT)ₙ, wherein n is 1, 2, or 3 (SEQ ID NO:312); GTTGCTTCTCTTAATTCC(invdT)ₙ, wherein n is 1, 2, or 3 (SEQ ID NO:313); ATGTTGCTTCTCT(invdT)ₙ, wherein n is 1, 2, or 3 (SEQ ID NO:314); or TTGCTTCTCTTA(invdT)ₙ, wherein n is 1, 2, or 3 (SEQ ID NO:315); with underlined nucleic acids representing locked nucleic acids. In some embodiments, a blocking nucleic acid of the present disclosure hybridizes with a wild-type EGFR locus corresponding with the locus of one or more DNA mutations at T790, C797, S768, V769, H773, or D770 of EGFR, e.g., DNA mutation(s) encoding a T790M, C797S, S768I, V769_D770insASV, H773_V774insH, D770_N771insG, or D770_N771insSVD mutated EGFR protein. In some embodiments, the blocking nucleic acid comprises the sequence CATCACGCAGCTCATG (invdT)ₙ, wherein n is 1, 2, or 3 (SEQ ID NO:316); TGCAGCTCATCACGCAGC (invdT)ₙ, wherein n is 1, 2, or 3 (SEQ ID NO:317); TCATCACGCAGCTCAT (invdT)ₙ, wherein n is 1, 2, or 3 (SEQ ID NO:318); TCATCACGCAGC (invdT)ₙ, wherein n is 1, 2, or 3 (SEQ ID NO:319); or CTCATCACGCAGC (invdT)ₙ, wherein n is 1, 2, or 3 (SEQ ID NO:320); with italicized nucleic acids representing locked nucleic acids. In some embodiments, the blocking nucleic acid comprises the sequence CATCACGCAGCTCATG (invdT)ₙ, wherein n is 1, 2, or 3 (SEQ ID NO:316); TGCAGCTCATCACGCAGC (invdT)ₙ, wherein n is 1, 2, or 3 (SEQ ID NO:317); TCATCACGCAGCTCAT (invdT)ₙ, wherein n is 1, 2, or 3 (SEQ ID NO:318); TCATCACGCAGC (invdT)ₙ, wherein n is 1, 2, or 3 (SEQ ID NO: 319); or CTCATCACGCAGC (invdT)ₙ, wherein n is 1, 2, or 3 (SEQ ID NO:320); with underlined nucleic acids representing locked nucleic acids. In some embodiments of any of the above embodiments, n is 1, 2, 3, 4, 5, 6, 7, 8, 9, or 10. In certain embodiments, n is 3. In some embodiments, a blocking nucleic acid of the present disclosure hybridizes with a wild-type EGFR locus corresponding with the locus of one or more DNA mutations at L858 or L861 of EGFR, e.g., DNA mutation(s) encoding an L858R mutated EGFR protein. In some embodiments, the blocking nucleic acid comprises the sequence CCAGCAGTTTGGCCAGCCCT (invdT)ₙ, wherein n is 1, 2, or 3 (SEQ ID NO:321); CCAGCAGTTTGGCCAGCCCT (invdT)ₙ, wherein n is 1, 2, or 3 (SEQ ID NO:322); CCAGCAGTTTGGCCAGCCCT (invdT)ₙ, wherein n is 1, 2, or 3 (SEQ ID NO:323); AGCAGTTTGGCCAGCC (invdT)ₙ, wherein n is 1, 2, or 3 (SEQ ID NO:324); or CCAGCAGTTTGGCCAGCCCT (invdT)ₙ, wherein n is 1, 2, or 3 (SEQ ID NO:325); with italicized nucleic acids representing locked nucleic acids. In some embodiments, the blocking nucleic acid comprises the sequence CCAGCAGTTTGGCCAGCCCT (invdT)ₙ, wherein n is 1, 2, or 3 (SEQ ID NO:321); CCAGCAGTTTGGCCAGCCCT (invdT)ₙ, wherein n is 1, 2, or 3 (SEQ ID NO:322); CCAGCAGTTTGGCCAGCCCT (invdT)ₙ, wherein n is 1, 2, or 3 (SEQ ID NO:323); AGCAGTTTGGCCAGCC (invdT)ₙ, wherein n is 1, 2, or 3 (SEQ ID NO:324); or CCAGCAGTTTGGCCAGCCCT (invdT)ₙ, wherein n is 1, 2, or 3 (SEQ ID NO:325); with underlined nucleic acids representing locked nucleic acids. In some embodiments, a blocking nucleic acid of the present disclosure hybridizes with a wild-type EGFR locus corresponding with the locus of one or more DNA mutations at T790 of EGFR, e.g., DNA mutation(s) encoding a T790M mutated EGFR protein. In some embodiments, the blocking nucleic acid comprises the sequence CATCACGCAGC (invdT)ₙ, wherein n is 1, 2, or 3 (SEQ ID NO:365); CATCACGCAG (invdT)ₙ, wherein n is 1, 2, or 3 (SEQ ID NO:366); ATCACGCAGC (invdT)ₙ, wherein n is 1, 2, or 3 (SEQ ID NO:367); CATCACGCAGC (invdT)ₙ, wherein n is 1, 2, or 3 (SEQ ID NO:368); or CATCACGCAG (invdT)ₙ, wherein n is 1, 2, or 3 (SEQ ID NO:369); with italicized nucleic acids representing locked nucleic acids. In some embodiments, the blocking nucleic acid comprises the sequence CATCACGCAGC (invdT)ₙ, wherein n is 1, 2, or 3 (SEQ ID NO:365); CATCACGCAG (invdT)ₙ, wherein n is 1, 2, or 3 (SEQ ID NO:366); ATCACGCAGC (invdT)ₙ, wherein n is 1, 2, or 3 (SEQ ID NO:367); CATCACGCAGC (invdT)ₙ, wherein n is 1, 2, or 3 (SEQ ID NO:368); or CATCACGCAG (invdT)ₙ, wherein n is 1, 2, or 3 (SEQ ID NO:369); with underlined nucleic acids representing locked nucleic acids. In some embodiments, a blocking nucleic acid of the present disclosure hybridizes with a wild-type EGFR locus corresponding with the locus of one or more DNA mutations at C797 of EGFR, e.g., DNA mutation(s) encoding a C797S (T>A or G>C) mutated EGFR protein. In some embodiments, the blocking nucleic acid comprises the sequence GGCTGCCTCCTG (invdT)ₙ, wherein n is 1, 2, or 3 (SEQ ID NO:416); CGGCTGCCTCCTG (invdT)ₙ, wherein n is 1, 2, or 3 (SEQ ID NO:417); CGGCTGCCTCCTG (invdT)ₙ, wherein n is 1, 2, or 3 (SEQ ID NO:418); TCGGCTGCCTCCTG (invdT)ₙ, wherein n is 1, 2, or 3 (SEQ ID NO:419); or TCGGCTGCCTCCT (invdT)ₙ, wherein n is 1, 2, or 3 (SEQ ID NO:420); with italicized nucleic acids representing locked nucleic acids. In some embodiments, the blocking nucleic acid comprises the sequence GGCTGCCTCCTG (invdT), wherein n is 1, 2, or 3 (SEQ ID NO:416); CGGCTGCCTCCTG (invdT)ₙ, wherein n is 1, 2, or 3 (SEQ ID NO:417); CGGCTGCCTCCTG (invdT)ₙ, wherein n is 1, 2, or 3 (SEQ ID NO:418); TCGGCTGCCTCCTG (invdT)ₙ, wherein n is 1, 2, or 3 (SEQ ID NO:419); or TCGGCTGCCTCCT (invdT)ₙ, wherein n is 1, 2, or 3 (SEQ ID NO:420); with underlined nucleic acids representing locked nucleic acids. In some embodiments of any of the above embodiments, n is 1, 2, 3, 4, 5, 6, 7, 8, 9, or 10. In certain embodiments, n is 3. In some embodiments, the blocking nucleic acid comprises a sequence of SEQ ID NOs:306-325, 365-369, and 416-420 but optionally includes a different pattern or type of modified nucleotide(s). In some embodiments, the blocking nucleic acid comprises a sequence of SEQ ID NOs: 306-325, 365-369, and 416-420 but includes a different 3′ terminal moiety.

In some embodiments, a blocking nucleic acid of the present disclosure hybridizes with a wild-type AKT1 locus corresponding with the locus of one or more DNA mutations at E17 of AKT1, e.g., DNA mutation(s) encoding an E17K mutated AKT1 protein. In some embodiments, the blocking nucleic acid comprises the sequence TGTACTCCCCTACA (invdT)ₙ, wherein n is 1, 2, or 3 (SEQ ID NO:382); GATGTACTCCCCT (invdT)ₙ, wherein n is 1, 2, or 3 (SEQ ID NO:383); ATGTACTCCCCTAC (invdT)ₙ, wherein n is 1, 2, or 3 (SEQ ID NO:384); GTACTCCCCTACA (invdT)ₙ, wherein n is 1, 2, or 3 (SEQ ID NO:385); or GATGTACTCCCCTACA (invdT)ₙ, wherein n is 1, 2, or 3 (SEQ ID NO:386); with italicized nucleic acids representing locked nucleic acids. In some embodiments, the blocking nucleic acid comprises the sequence TGTACTCCCCTACA (invdT)ₙ, wherein n is 1, 2, or 3 (SEQ ID NO:382); GATGTACTCCCCT (invdT)ₙ, wherein n is 1, 2, or 3 (SEQ ID NO:383); ATGTACTCCCCTAC (invdT)ₙ, wherein n is 1, 2, or 3 (SEQ ID NO:384); GTACTCCCCTACA (invdT)ₙ, wherein n is 1, 2, or 3 (SEQ ID NO:385); or GATGTACTCCCCTACA (invdT)ₙ, wherein n is 1, 2, or 3 (SEQ ID NO:386); with underlined nucleic acids representing locked nucleic acids. In some embodiments of any of the above embodiments, n is 1, 2, 3, 4, 5, 6, 7, 8, 9, or 10. In certain embodiments, n is 3. In some embodiments, the blocking nucleic acid comprises a sequence of SEQ ID NOs:382-386 but optionally includes a different pattern or type of modified nucleotide(s). In some embodiments, the blocking nucleic acid comprises a sequence of SEQ ID NOs: 382-386 but includes a different 3′ terminal moiety.

In some embodiments, a blocking nucleic acid of the present disclosure hybridizes with a wild-type MEK1 locus corresponding with the locus of one or more DNA mutations at K57 of MEK1, e.g., DNA mutation(s) encoding a K57N mutated MEK1 protein. In some embodiments, the blocking nucleic acid comprises the sequence TCTGCTTCTGGGTAAG (invdT)ₙ, wherein n is 1, 2, or 3 (SEQ ID NO:399); TTCTGCTTCTGGGTAAGA (invdT)ₙ, wherein n is 1, 2, or 3 (SEQ ID NO:400); CACCTTCTGCTTCTGGG (invdT)ₙ, wherein n is 1, 2, or 3 (SEQ ID NO:401); TCTGCTTCTGGGTA (invdT)ₙ, wherein n is 1, 2, or 3 (SEQ ID NO:402); or CACCTTCTGCTTCTGGGTAAGA (invdT)ₙ, wherein n is 1, 2, or 3 (SEQ ID NO:403); with italicized nucleic acids representing locked nucleic acids. In some embodiments, the blocking nucleic acid comprises the sequence TCTGCTTCTGGGTAAG (invdT)ₙ, wherein n is 1, 2, or 3 (SEQ ID NO:399); TTCTGCTTCTGGGTAAGA (invdT)ₙ, wherein n is 1, 2, or 3 (SEQ ID NO:400); CACCTTCTGCTTCTGGG (invdT)ₙ, wherein n is 1, 2, or 3 (SEQ ID NO:401); TCTGCTTCTGGGTA (invdT)ₙ, wherein n is 1, 2, or 3 (SEQ ID NO:402); or CACCTTCTGCTTCTGGGTAAGA (invdT)ₙ, wherein n is 1, 2, or 3 (SEQ ID NO:403); with underlined nucleic acids representing locked nucleic acids. In some embodiments of any of the above embodiments, n is 1, 2, 3, 4, 5, 6, 7, 8, 9, or 10. In certain embodiments, n is 3. In some embodiments, the blocking nucleic acid comprises a sequence of SEQ ID NOs:399-403 but optionally includes a different pattern or type of modified nucleotide(s). In some embodiments, the blocking nucleic acid comprises a sequence of SEQ ID NOs:399-403 but includes a different 3′ terminal moiety.

Hybridization

In some embodiments, the methods of the present disclosure include hybridizing amplified DNA with one or more probes specific for a DNA or RNA mutation of the present disclosure. In some embodiments, the methods include hybridizing amplified DNA with at least seven probes, comprising one or more probes specific for a DNA mutation in each of the KRAS, NRAS, PIK3CA, BRAF, EGFR, AKT1, MEK1, and HER2 genes (e.g., one or more probes representing a mutation in each gene) and/or hybridizing amplified DNA with at least five probes, comprising one or more probes specific for an RNA mutation in each of the ALK, ROS, RET, NTRK1, and cMET genes (e.g., one or more probes representing a mutation in each gene).

As used herein, a probe may refer to an oligonucleotide that is capable of hybridization with at least a portion of the locus of a DNA or RNA mutation of interest. For example, a probe may include a single-stranded oligonucleotide that is able to base pair with most or all of the base pairs of a single-stranded DNA template that includes a DNA mutation of interest, or a single-stranded DNA template (e.g., generated from RNA and subsequently cDNA) that includes an RNA mutation of interest. For specific detection of a mutation, the probe is able to hybridize with a locus bearing the DNA or RNA mutation, but not with the corresponding wild-type locus (cf. FIGS. 4 & 5). Conditions suitable for hybridization of a probe with amplified DNA are known in the art (e.g., as referenced in the materials cited herein) and exemplified infra.

In some embodiments, a probe of the present disclosure is coupled to an encoded microcarrier of the present disclosure, e.g., as described in section IV. Exemplary methods for coupling a polynucleotide probe to a microcarrier surface are known in the art and provided in section IV. For multiplex assays, each type of probe can be coupled to a microcarrier with a particular identifier corresponding to the probe type. Advantageously, this allows the user to correlate a signal detected from the probe with the probe's identity, enabling multiplex assays in which multiple probes are utilized. In some embodiments, a probe of the present disclosure comprises a 5′ modification, e.g., a 5′ amino modifier C6.

In some embodiments, a probe of the present disclosure comprises (1) a sequence that hybridizes with at least a portion of the locus of a DNA or RNA mutation of interest; and (2) one or more additional nucleotides. The one or more additional nucleotides may be used, e.g., to couple the probe to the microcarrier surface and/or to provide spacing to reduce steric hindrance between the microcarrier surface and the amplified DNA during hybridization. In some embodiments, the one or more additional nucleotides are at the 5′ end of the probe sequence. In other embodiments, the one or more additional nucleotides are at the 3′ end of the probe sequence. In some embodiments, the one or more additional nucleotides are adenine or thymine nucleotides. Advantageously, this reduces the affinity of non-specific binding. In some embodiments, a probe of the present disclosure comprises 1 or more, 2 or more, 3 or more, 4 or more, 5 or more, 6 or more, 7 or more, or 8 or more adenine or thymine nucleotides at the 5′ end. In some embodiments, a probe of the present disclosure comprises 4, 5, 6, 7, or 8 adenine or thymine nucleotides at the 5′ end. In some embodiments, a probe of the present disclosure comprises at least 20, at least 24, at least 25, or at least 30 total nucleotides. Exemplary probe sequences are provided below.

In some embodiments, one or more probe(s) specific for a DNA mutation in a KRAS gene as described herein is/are used. For example; a probe comprising the sequence TAGTTGGAGCT (SEQ ID NO:38), TGTGGTAGTTG (SEQ ID NO:40), TGATGGCGTAG (SEQ ID NO: 42), TGGAGCTGATGGC (SEQ ID NO:44), or GCGTAGGCAAG (SEQ ID NO:46) can be used to detect a mutation encoding a G12D mutated KRAS protein; a probe comprising the sequence CTGTTGGCGTAGG (SEQ ID NO:48), GTAGTTGGAGCTG (SEQ ID NO:50), TGGAGCTGTTGGC (SEQ ID NO:52), TTGTGGTAGTTGG (SEQ ID NO:54), or GGCGTAGGCAAGA (SEQ ID NO:56) can be used to detect a mutation encoding a G12V mutated KRAS protein; a probe comprising the sequence TAGTTGGAGCTT (SEQ ID NO:58), GCGTAGGCAAGA (SEQ ID NO:60), GGAGCTTGTGGC (SEQ ID NO: 62), TTGTGGCGTAGG (SEQ ID NO:64), and/or TGTGGTAGTTGG (SEQ ID NO:66) can be used to detect a mutation encoding a G12C mutated KRAS protein. As described supra, in some embodiments, one or more probes of the present disclosure can comprise four, five, six, seven, or eight or more nucleotides (e.g., adenines or thymines) at its 5′ end. In certain embodiments, a probe comprising the sequence TTTTTTTTTTTTAATAGTTGGAGCT (SEQ ID NO:39), TTTTTTTTTTTTAATGTGGTAGTTG (SEQ ID NO:41), TTTTTTTTTTTTAATGATGGCGTAG (SEQ ID NO:43), TTTTTTTTTTTATGGAGCTGATGGC (SEQ ID NO:45), or TTTTTTTTTTTTAAGCGTAGGCAAG (SEQ ID NO:47) can be used to detect a mutation encoding a G12D mutated KRAS protein. In certain embodiments, a probe comprising the sequence TTTTTTTTTTTACTGTTGGCGTAGG (SEQ ID NO:49), TTTTTTTTTTTAGTAGTTGGAGCTG (SEQ ID NO:51), TTTTTTTTTTTATGGAGCTGTTGGC (SEQ ID NO:53), TTTTTTTTTTTATTGTGGTAGTTGG (SEQ ID NO:55), or TTTTTTTTTTTAGGCGTAGGCAAGA (SEQ ID NO:57) can be used to detect a mutation encoding a G12V mutated KRAS protein. In certain embodiments, a probe comprising the sequence TTTTTTTTTTTAATAGTTGGAGCTT (SEQ ID NO:59), TTTTTTTTTTTAAGCGTAGGCAAGA (SEQ ID NO:61), TTTTTTTTTTTAAGGAGCTTGTGGC (SEQ ID NO: 63), TTTTTTTTTTTAATTGTGGCGTAGG (SEQ ID NO: 65), or TTTTTTTTTTAATGTGGTAGTTGG (SEQ ID NO:67) can be used to detect a mutation encoding a G12C mutated KRAS protein. Probes comprising these sequences exclusive of the 5′ adenine and/or thymines are also contemplated.

In certain embodiments, a probe comprising the sequence TTTTTTTTTTTGACATACTGGATACAG (SEQ ID NO:69), TTTTTTTTTTTACTGGATACAGCTGGA (SEQ ID NO:71), TTTTTTTTTTTACTAGAAGAGTACAGT (SEQ ID NO:73), TTTTTTTTTTTATACAGCTGGACTAGA (SEQ ID NO:75), or TTTTTTTTTTTGCTGGACTAGAAGAGT (SEQ ID NO:77) can be used to detect a mutation encoding a Q61L (A>T) mutated NRAS protein. Probes comprising these sequences exclusive of the 5′ adenine and/or thymines are also contemplated.

In some embodiments, one or more probe(s) specific for a DNA mutation in a BRAF gene as described herein is/are used. For example, a probe comprising the sequence TTTGGTCTAGCTACAGA (SEQ ID NO:79), CTACAGAGAAATCTCGA (SEQ ID NO:81), GTGATTTTGGTCTAGCT (SEQ ID NO:83), or TCTAGCTACAGAGAAAT (SEQ ID NO:85) can be used to detect a mutation encoding a V600E mutated BRAF protein. As described supra, in some embodiments, one or more probes of the present disclosure can comprise four, five, six, seven, or eight or more nucleotides (e.g., adenines or thymines) at its 5′ end. In certain embodiments, a probe comprising the sequence TTTTTTAATTGAGAAATCTCGATGGAG (SEQ ID NO:78), TTTTTTAATTTTTGGTCTAGCTACAGA (SEQ ID NO:80), TTTTTTAATTCTACAGAGAAATCTCGA (SEQ ID NO:82), TTTTTTAATTGTGATTTTGGTCTAGCT (SEQ ID NO:84), or TTTTTTAATTTCTAGCTACAGAGAAAT (SEQ ID NO:86) can be used to detect a mutation encoding a V600E mutated BRAF protein. Probes comprising these sequences exclusive of the 5′ adenine and/or thymines are also contemplated.

In some embodiments, one or more probe(s) specific for a DNA mutation in a PIK3CA gene as described herein is/are used. For example, a probe comprising the sequence GCTCAGTGATTTTAG (SEQ ID NO:87), TGCTCAGTGATTTT (SEQ ID NO:89), GCTCAGTGATTTTAG (SEQ ID NO:91), CCTGCTCAGTGATTTTA (SEQ ID NO:93), or CTCAGTGATTTTAGA (SEQ ID NO:95) can be used to detect a mutation encoding an E542K mutated PIK3CA protein; a probe comprising the sequence TTCTCCTGCTTA (SEQ ID NO:97), CTCCTGCTTAGT (SEQ ID NO:99), TCTCCTGCTTAG (SEQ ID NO:101), TCCTGCTTAGTG (SEQ ID NO:103), or CTCCTGCTTAGTGA (SEQ ID NO:105) can be used to detect a mutation encoding an E545K mutated PIK3CA protein; and/or a probe comprising the sequence GATGCACGTCATG (SEQ ID NO:107), TGAATGATGCACG (SEQ ID NO:109), TGATGCACGTC (SEQ ID NO:111), AATGATGCACGTCA (SEQ ID NO:113), or AATGATGCACGTC (SEQ ID NO:115) can be used to detect a mutation encoding an H1047R mutated PIK3CA protein. As described supra, in some embodiments, one or more probes of the present disclosure can comprise four, five, six, seven, or eight or more nucleotides (e.g., adenines or thymines) at its 5′ end. In certain embodiments, a probe comprising the sequence TTTTTTTTTAGCTCAGTGATTTTAG (SEQ ID NO:88), TTTTTTTTTTGCTCAGTGATTTT (SEQ ID NO:90), TTTTTTTTTAGCTCAGTGATTTTAG (SEQ ID NO:92), TTTTTTTCCTGCTCAGTGATTTTA (SEQ ID NO: 94), or TTTTTTTTTTTCTCAGTGATTTTAGA (SEQ ID NO:96) can be used to detect a mutation encoding an E542K mutated PIK3CA protein. In certain embodiments, a probe comprising the sequence TTTTTTTTTTTTTCTCCTGCTTA (SEQ ID NO:98), TTTTTTTTTTTTTCTCCTGCTTAGT (SEQ ID NO:100), TTTTTTTTTTTATCTCCTGCTTAG (SEQ ID NO:102), TTTTTTTTTTTTTTTCCTGCTTAGTG (SEQ ID NO:104), or TTTTTTTTTTTTTCTCCTGCTTAGTGA (SEQ ID NO:106) can be used to detect a mutation encoding an E545K mutated PIK3CA protein. In certain embodiments. In certain embodiments, a probe comprising the sequence TTTTTTTTTTTTTTTGATGCACGTCATG (SEQ ID NO:108), TTTTTTTTTTTGAATGATGCACG (SEQ ID NO:110), TTTTTTTTTTTTTGATGCACGTC (SEQ ID NO:112), TTTTTTTTTTTTAATGATGCACGTCA (SEQ ID NO:114), or TTTTTTTTTTTTAATGATGCACGTC (SEQ ID NO:116) can be used to detect a mutation encoding an H1047R mutated PIK3CA protein. Probes comprising these sequences exclusive of the 5′ adenine and/or thymines are also contemplated.

In some embodiments, one or more probe(s) specific for a DNA mutation in an EGFR gene as described herein is/are used. For example, a probe comprising the sequence ATGGCCATCTTGG (SEQ ID NO:421), GGCCATCTTGGA (SEQ ID NO:423), GATGGCCATCTTG (SEQ ID NO:425), TGATGGCCATCTTG (SEQ ID NO:427), or TGGCCATCTTGG (SEQ ID NO:429) can be used to detect a mutation encoding an S768I mutated EGFR protein; a probe comprising the sequence GTGATGGCCGG (SEQ ID NO:431), TGATGGCCGGCG (SEQ ID NO:433), GTGATGGCCGGCGT (SEQ ID NO:435), GATGGCCGGCGT (SEQ ID NO:437), or GATGGCCCGCGTG (SEQ ID NO:439) can be used to detect a mutation encoding a V769_D770insASV, D770_N771insSVD, or V769_D770insASV mutated EGFR protein; a probe comprising the sequence AACCCCCATCACGT (SEQ ID NO:441), GACAACCCCCATCACG (SEQ ID NO:443), CGTGGACAACCCCCATCA (SEQ ID NO:445), CCCATCACGTGT (SEQ ID NO:447), or TGGACAACCCCCATCAC (SEQ ID NO:449) can be used to detect a mutation encoding an H773_V774insH mutated EGFR protein; a probe comprising the sequence GCCAGCGTGGACGG (SEQ ID NO:451), CGTGGACGGTAACC (SEQ ID NO:453), GACGGTAACCCCC (SEQ ID NO:455), CCAGCGTGGACGGT (SEQ ID NO:457), or GCCAGCGTGGACGGTA (SEQ ID NO:459) can be used to detect a mutation encoding a D770_N771insG mutated EGFR protein; a probe comprising the sequence CCAGGAGGCTGCCG (SEQ ID NO:461), CAGGAGGCTGCCGA (SEQ ID NO:463), TCCAGGAGGCTGCC (SEQ ID NO:465), CCAGGAGGCTGCC (SEQ ID NO:467), or CAGGAGGCTGCC (SEQ ID NO:469) can be used to detect a mutation encoding a C797S (T>A) mutated EGFR protein; a probe comprising the sequence CCAGGAGGGAGCC (SEQ ID NO:471), CCAGGAGGGAGCCG (SEQ ID NO:473), TCCAGGAGGGAGCC (SEQ ID NO:475), CAGGAGGGAGCCG (SEQ ID NO:477), or CAGGAGGGAGCCGA (SEQ ID NO:479) can be used to detect a mutation encoding a C797S (G>C) mutated EGFR protein; a probe comprising the sequence TCAAAGTGCTGGCCTC (SEQ ID NO:117), AGATCAAAGTGCTGGCCTCCG (SEQ ID NO:119), AAAGTGCTGGCCT (SEQ ID NO:121), AGTGCTGGCCT (SEQ ID NO:123), or AAGTGCTGGCCTC (SEQ ID NO:125) can be used to detect a mutation encoding a G719A mutated EGFR protein; a probe comprising the sequence AATCAAAACATCTCCGAAAG (SEQ ID NO:128), CAAAACATCTCCG (SEQ ID NO:130), AACATCTCCG (SEQ ID NO:132), AAACATCTCCGAAAGCC (SEQ ID NO:134), AATCAAGACATCTCCGA (SEQ ID NO:136), GCAATCAAGACATCTCCGA (SEQ ID NO:138), AATCAAGACATCTC (SEQ ID NO:140), AATCAAGACATCTCCGAAAGC (SEQ ID NO:142), or CAAGACATCTCCGA (SEQ ID NO:144) can be used to detect a mutation encoding an E746_A750del mutated EGFR protein; and/or a probe comprising the sequence ATTTTGGGCGGGCC (SEQ ID NO:151), TTGGGCGGGCCAAA (SEQ ID NO:153), GCGGGCCAAACT (SEQ ID NO:155), GGGCGGGCCAAACT (SEQ ID NO:157), or TGGGCGGGCCA (SEQ ID NO:159) can be used to detect a mutation encoding an L858R mutated EGFR protein. As described supra, in some embodiments, one or more probes of the present disclosure can comprise four, five, six, seven, or eight or more nucleotides (e.g., adenines or thymines) at its 5′ end. In certain embodiments, a probe comprising the sequence TTTTTTTTTTTGAGATGCATGATGA (SEQ ID NO:352), TTTTTTTTTTGAGATGCATGATGAG (SEQ ID NO:353). TTTTTTTATGAGATGCATGATGAG (SEQ ID NO:354), TTTTTTTTTTTGAGCTGCATGATGA (SEQ ID NO:355), or TTTTTTTTCATGAGATGCATGATGA (SEQ ID NO:356) can be used to detect a mutation encoding a T790M mutated EGFR protein. In certain embodiments, a probe comprising the sequence TTTTTTTTTATGGCCATCTTGG (SEQ ID NO:422), TTTTTTTTTTAGGCCATCTTGGA (SEQ ID NO:424), TTTTTTTAGATGGCCATCTTG (SEQ ID NO:426), TTTTTTTTGATGGCCATCTTG (SEQ ID NO:428), or TTTTTTTTTTTGGCCATCTTGG (SEQ ID NO:430) can be used to detect a mutation encoding an S768I mutated EGFR protein. In certain embodiments, a probe comprising the sequence TTTTTTTTTTTGTGATGGCCGG (SEQ ID NO:432), TTTTTTTTTTTTTGATGGCCGGCG (SEQ ID NO:434), TTTTTTTTTTTGTGATGGCCGGCGT (SEQ ID NO:436), TTTTTTTTTTTTTGATGGCCGGCGT (SEQ ID NO:438), or TTTTTTTTTTTTTGATGGCCCGCGTG (SEQ ID NO:440) can be used to detect a mutation encoding a V769_D770insASV, D770_N771insSVD, or V769_D770insASV mutated EGFR protein. In certain embodiments, a probe comprising the sequence TTTTTTTTTTTAACCCCCATCACGT (SEQ ID NO:442), TTTTTTTTGACAACCCCCATCACG (SEQ ID NO:444), TTTTCGTGGACAACCCCCATCA (SEQ ID NO:446), TTTTTTTTTTTTCCCATCACGTGT (SEQ ID NO:448), or TTTTTTTTGGACAACCCCCATCAC (SEQ ID NO:450) can be used to detect a mutation encoding an H773_V774insH mutated EGFR protein. In certain embodiments, a probe comprising the sequence TTTTTTTTTTTGCCAGCGTGGACGG (SEQ ID NO:452), TTTTTTTTTTTCGTGGACGGTAACC (SEQ ID NO:454), TTTTTTTTTTTGACGGTAACCCCC (SEQ ID NO:456), TTTTTTTTTTCCAGCGTGGACGGT (SEQ ID NO:458), or TTTTTTTGCCAGCGTGGACGGTA (SEQ ID NO:460) can be used to detect a mutation encoding a D770_N771insG mutated EGFR protein. In certain embodiments, a probe comprising the sequence TTTTTTTTTTTACCAGGAGGCTGCCG (SEQ ID NO:462), TTTTTTTTTTTACAGGAGGCTGCCGA (SEQ ID NO:464), TTTTTTTTTTTATCCAGGAGGCTGCC (SEQ ID NO:466), TTTTTTTTTTTACCAGGAGGCTGCC (SEQ ID NO:468), or TTTTTTTTTTTACAGGAGGCTGCC (SEQ ID NO:470) can be used to detect a mutation encoding a C797S (T>A) mutated EGFR protein. In certain embodiments, a probe comprising the sequence TTTTTTTTTTTACCAGGAGGGAGCC (SEQ ID NO:472), TTTTTTTTTTTACCAGGAGGGAGCCG (SEQ ID NO:474), TTTTTTTTTTTATCCAGGAGGGAGCC (SEQ ID NO:476), TTTTTTTTTTTACAGGAGGGAGCCG (SEQ ID NO:478), or TTTTTTTTTTTACAGGAGGGAGCCGA (SEQ ID NO:480) can be used to detect a mutation encoding a C797S (G>C) mutated EGFR protein. In certain embodiments, a probe comprising the sequence TTTTTTTTTTCAAAGTGCTGGCCTC (SEQ ID NO:118), TTTTTTAGATCAAAGTGCTGGCCTCCG (SEQ ID NO:120), TTTTTTTTTTTAAAGTGCTGGCCT (SEQ ID NO: 122), TTTTTTTTTTTTTAGTGCTGGCCT (SEQ ID NO:124), or TTTTTTTTTTTTAAGTGCTGGCCTC (SEQ ID NO:126) can be used to detect a mutation encoding a G719A mutated EGFR protein. In certain embodiments, a probe comprising the sequence TTTTTTTTTAATCAAAACATCTCCG (SEQ ID NO:127), TTTTTTTTTAATCAAAACATCTCCGAAAG (SEQ ID NO:129), TTTTTTTTTTTACAAAACATCTCCG (SEQ ID NO:131), TTTTTTTTTTTTTTTAACATCTCCG (SEQ ID NO:133), TTTTTTTTTTTTTTAAACATCTCCGAAAGCC (SEQ ID NO:135), TTTTTTTTAATCAAGACATCTCCGA (SEQ ID NO:137). TTTTTTGCAATCAAGACATCTCCGA (SEQ ID NO:139), TTTTTTTTAATCAAGACATCTC (SEQ ID NO:141), TTTTTTTTAATCAAGACATCTCCGAAAGC (SEQ ID NO:143), or TTTTTTTTTTTCAAGACATCTCCGA (SEQ ID NO:145) can be used to detect a mutation encoding an E746_A750del mutated EGFR protein. In certain embodiments, a probe comprising the sequence TTTTTTTATTTTGGGCGGGCC (SEQ ID NO:152), TTTTTTTTAATTGGGCGGGCCAAA (SEQ ID NO:154), TTTTTTTAAAAAAGCGGGCCAAACT (SEQ ID NO:156), TTTTTTTTAAAAGGGCGGGCCAAACT (SEQ ID NO:158), or TTTTTTTTAAATGGGCGGGCCA (SEQ ID NO:160) can be used to detect a mutation encoding an L858R mutated EGFR protein. In certain embodiments. Probes comprising these sequences exclusive of the 5′ adenine and/or thymines are also contemplated.

In some embodiments, one or more probe(s) specific for a DNA mutation in an AKT1 gene as described herein is/are used. For example, a probe comprising the sequence TGTAGGGAAGTACA (SEQ ID NO:370), TCTGTAGGGAAGTAC (SEQ ID NO:372), GTCTGTAGGGAAGTACAT (SEQ ID NO:374), CCGCACGTCTGTAGGGA (SEQ ID NO:376), or ACGTCTGTAGGGAAGTA (SEQ ID NO:378) can be used to detect a mutation encoding an E17K mutated AKT1 protein. As described supra, in some embodiments, one or more probes of the present disclosure can comprise four, five, six, seven, or eight or more nucleotides (e.g., adenines or thymines) at its 5′ end. In certain embodiments, a probe comprising the sequence TTTTTTTTTTTTTTGTAGGGAAGTACA (SEQ ID NO:371), TTTTTTTTTTTTCTGTAGGGAAGTAC (SEQ ID NO:373), TTTTTTTGTCTGTAGGGAAGTACAT (SEQ ID NO:375), TTTTTTTCCGCACGTCTGTAGGGA (SEQ ID NO:377), or TTTTTTTTACGTCTGTAGGGAAGTA (SEQ ID NO:379) can be used to detect a mutation encoding an E17K mutated AKT1 protein. Probes comprising these sequences exclusive of the 5′ adenine and/or thymines are also contemplated.

In some embodiments, one or more probe(s) specific for a DNA mutation in a MEK1 gene as described herein is/are used. For example, a probe comprising the sequence TTACCCAGAATCAGAA (SEQ ID NO:387), CCAGAATCAGAAGGTG (SEQ ID NO:389), TTCTTACCCAGAATCA (SEQ ID NO:391), CCTTTCTTACCCAGAATC (SEQ ID NO: 393), or CAGAATCAGAAGGTGG (SEQ ID NO:395) can be used to detect a mutation encoding a K57N mutated MEK1 protein. As described supra, in some embodiments, one or more probes of the present disclosure can comprise four, five, six, seven, or eight or more nucleotides (e.g., adenines or thymines) at its 5′ end. In certain embodiments, a probe comprising the sequence TTTTTAAATTTACCCAGAATCAGAA (SEQ ID NO:388), TTTTTAAATCCAGAATCAGAAGGTG (SEQ ID NO:390), TTTTTAAATTTCTTACCCAGAATCA (SEQ ID NO:392), TTTTTAAATCCTTTCTTACCCAGAATC (SEQ ID NO:394), or TTTTTAAATCAGAATCAGAAGGTGG (SEQ ID NO:396) can be used to detect a mutation encoding a K57N mutated MEK1 protein. Probes comprising these sequences exclusive of the 5′ adenine and/or thymines are also contemplated.

In some embodiments, one or more probe(s) specific for a DNA mutation in a HER2 gene as described herein is/are used. For example, a probe comprising the sequence ATACGTGATGTCTTAC (SEQ ID NO:404), ACGTGATGGCTTACGT (SEQ ID NO:406), AAGCATACGTGATGGCT (SEQ ID NO:408), GCATACGTGATGGCTT (SEQ ID NO:410), or GCATACGTGATGGCTTA (SEQ ID NO:412) can be used to detect a mutation encoding an A775_G776insYVMA mutated HER2 protein. As described supra, in some embodiments, one or more probes of the present disclosure can comprise four, five, six, seven, or eight or more nucleotides (e.g., adenines or thymines) at its 5′ end. In certain embodiments, a probe comprising the sequence TTTTTTTTTATACGTGATGTCTTAC (SEQ ID NO:405), TTTTTTTTTTTACGTGATGGCTTACGT (SEQ ID NO:407), TTTTTAAGCATACGTGATGGCT (SEQ ID NO:409), TTTTTTTGCATACGTGATGGCTT (SEQ ID NO:411), or TTTTTTTGCATACGTGATGGCTTA (SEQ ID NO:413) can be used to detect a mutation encoding an A775_G776insYVMA mutated HER2 protein. Probes comprising these sequences exclusive of the 5′ adenine and/or thymines are also contemplated.

In some embodiments, one or more probe(s) specific for an RNA mutation in an ALK gene (e.g., resulting in an EML4-ALK fusion gene) as described herein is/are used. For example, a probe comprising the sequence AAAGGACCTAAAGTGT (SEQ ID NO:161), CCTAAAGTGTACCGC (SEQ ID NO:163), GGGAAAGGACCTAAAG (SEQ ID NO:165), AGTGTACCGCCGGAA (SEQ ID NO:167), or TACCGCCGGAAGCACC (SEQ ID NO:169) can be used to detect an E13;A20 ALK mutation; a probe comprising the sequence GACTATGAAATATTGTAC (SEQ ID NO:171), GAAATATTGTACTTGTAC (SEQ ID NO:173), TATTGTACTTGTACCGCC (SEQ ID NO:175), TGTACCGCCGGAAGCAC (SEQ ID NO:177), or CCGCCGGAAGCACCAGGA (SEQ ID NO:179) can be used to detect an E20;A20 ALK mutation; a probe comprising the sequence TGTCATCATCAACCAA (SEQ ID NO:181), ATGTCATCATCAACC (SEQ ID NO:183), GTGTACCGCCGGAAGC (SEQ ID NO:185), TCAACCAAGTGTACCG (SEQ ID NO:187). TACCGCCGGAAGCACCA (SEQ ID NO:189), CGAAAAAAACAGCCAA (SEQ ID NO:191), TCGCGAAAAAAACAGC (SEQ ID NO:193), GTGTACCGCCGGAAGC (SEQ ID NO:195), TACCGCCGGAAGCACC (SEQ ID NO:197), or ACAGCCAAGTGTACCG (SEQ ID NO:199) can be used to detect an E6;A20 ALK mutation. As described supra, in some embodiments, one or more probes of the present disclosure can comprise four, five, six, seven, or eight or more nucleotides (e.g., adenines or thymines) at its 5′ end. In certain embodiments, a probe comprising the sequence TTTTTTTTTTAAAGGACCTAAAGTGT (SEQ ID NO:162), TTTTTTTTTTCCTAAAGTGTACCGC (SEQ ID NO:164), TTTTTTTTTTGGGAAAGGACCTAAAG (SEQ ID NO:166), TTTTTTTTTTAGTGTACCGCCGGAA (SEQ ID NO:168), or TTTTTTTTTTTACCGCCGGAAGCACC (SEQ ID NO:170) can be used to detect an E13;A20 ALK mutation. In certain embodiments, a probe comprising the sequence TTTTTTTTTTTTGACTATGAAATATTGTAC (SEQ ID NO:172), TTTTTTTTTTTTGAAATATTGTACTTGTAC (SEQ ID NO:174), TTTTTTTTTTTTTATTGTACTTGTACCGCC (SEQ ID NO:176), TTTTTTTTTTTTTTGTACCGCCGGAAGCAC (SEQ ID NO:178), or TTTTTTTTTTTTCCGCCGGAAGCACCAGGA (SEQ ID NO:180) can be used to detect an E20;A20 ALK mutation. In certain embodiments, a probe comprising the sequence TTTTTTTTTTTTTTTGTCATCATCAACCAA (SEQ ID NO:182), TTTTTTTTTTTTTTATGTCATCATCAACC (SEQ ID NO:184), TTTTTTTTTTTTTTGTGTACCGCCGGAAGC (SEQ ID NO:186), TTTTTTTTTTTTTTTCAACCAAGTGTACCG (SEQ ID NO:188), TTTTTTTTTTTTTTTACCGCCGGAAGCACCA (SEQ ID NO:190), TTTTTTTTTTTTTCGAAAAAAACAGCCAA (SEQ ID NO:192), TTTTTTTTTTTTTTCGCGAAAAAAACAGC (SEQ ID NO:194), TTTTTTTTTTTTTGTGTACCGCCGGAAGC (SEQ ID NO: 196), TTTTTTTTTTTTTTACCGCCGGAAGCACC (SEQ ID NO: 198), or TTTTTTTTTTTTTTACAGCCAAGTGTACCG (SEQ ID NO:200) can be used to detect an E6;A20 ALK mutation.

In some embodiments, one or more probe(s) specific for an RNA mutation in an ROS gene (e.g., resulting in a CD74-ROS or SLC34A2-ROS fusion gene) as described herein is/are used. For example, a probe comprising the sequence ACTGACGCTCCACCGAAA (SEQ ID NO:201), CCACTGACGCTCCACCGA (SEQ ID NO:203), GCTGGAGTCCCAAATAAAC (SEQ ID NO:205), GGAGTCCCAAATAAACCAG (SEQ ID NO:207), CACCGAAAGCTGGAGTCCC (SEQ ID NO:209), CCGAAAGATGATTTT (SEQ ID NO:211), GACGCTCCACCGAAA (SEQ ID NO:213), ACTGACGCTCCACCGA (SEQ ID NO:215), GATGATTTTTGGATA (SEQ ID NO:217), or TGATTTTTGGATACCA (SEQ ID NO:219) can be used to detect a CD74-ROS mutation; and/or a probe comprising the sequence AGCGCCTTCCAGCTGGTTGGA (SEQ ID NO:221), CTGGTTGGAGCTGGAGTCCC (SEQ ID NO:223), AGTAGCGCCTTCCAGCTGGTTG (SEQ ID NO:225), GCTGGAGTCCCAAATAAACCA (SEQ ID NO:227), GGAGTCCCAAATAAACCAGG (SEQ ID NO:229), GCGCCTTCCAGCTGGTTG (SEQ ID NO:231), GTAGCGCCTTCCAGCTGGT (SEQ ID NO:233), TGGTTGGAGATGATTTTT (SEQ ID NO:235), GATGATTTTTGGATACCAG (SEQ ID NO:237), or TGATTTTTGGATACCA (SEQ ID NO:239) can be used to detect a mutation encoding an SLC34A2-ROS mutation. As described supra, in some embodiments, one or more probes of the present disclosure can comprise four, five, six, seven, or eight or more nucleotides (e.g., adenines or thymines) at its 5′ end. In certain embodiments, a probe comprising the sequence TTTTTTTTTTTACTGACGCTCCACCGAAA (SEQ ID NO:202), TTTTTTTTTTTCCACTGACGCTCCACCGA (SEQ ID NO:204), TTTTTTTTTTTGCTGGAGTCCCAAATAAAC (SEQ ID NO:206), TTTTTTTTTTGGAGTCCCAAATAAACCAG (SEQ ID NO:208), TTTTTTTTTTTCACCGAAAGCTGGAGTCCC (SEQ ID NO:210), TTTTTTTTTTTTCCGAAAGATGATTTT (SEQ ID NO:212), TTTTTTTTTTTTGACGCTCCACCGAAA (SEQ ID NO:214), TTTTTTTTTTTTACTGACGCTCCACCGA (SEQ ID NO:216), TTTTTTTTTTTTGATGATTTTTGGATA (SEQ ID NO:218), or TTTTTTTTTTTTTGATTTTTGGATACCA (SEQ ID NO:220) can be used to detect a CD74-ROS mutation. In certain embodiments, a probe comprising the sequence TTTTTTTTTTTTAGCGCCTTCCAGCTGGTTGGA (SEQ ID NO:222), TTTTTTTTTTTTCTGGTTGGAGCTGGAGTCCC (SEQ ID NO:224), TTTTTTTTTTTTAGTAGCGCCTTCCAGCTGGTTG (SEQ ID NO:226), TTTTTTTTTTTTGCTGGAGTCCCAAATAAACCA (SEQ ID NO:228), TTTTTTTTTTTTGGAGTCCCAAATAAACCAGG (SEQ ID NO:230), TTTTTTTTTTGCGCCTTCCAGCTGGTTG (SEQ ID NO:232), TTTTTTTTTTGTAGCGCCTTCCAGCTGGT (SEQ ID NO:234), TTTTTTTTTTTGGTTGGAGATGATTTTT (SEQ ID NO:236), TTTTTTTTTTGATGATTTTTGGATACCAG (SEQ ID NO:238), or TTTTTTTTTTTGATTTTTGGATACCA (SEQ ID NO:240) can be used to detect an SLC34A2-ROS mutation. Probes comprising these sequences exclusive of the 5′ adenine and/or thymines are also contemplated.

In some embodiments, one or more probe(s) specific for an RNA mutation in a RET gene (e.g., resulting in a KIFSB-RET fusion gene) as described herein is/are used. For example, a probe comprising the sequence GTGGGAAATAATGATGTAAA (SEQ ID NO:241), CTGTGGGAAATAATGATGTA (SEQ ID NO:243), GATCCACTGTGCGACGAGCT (SEQ ID NO:245), TGATGTAAAGATCCACTGTG (SEQ ID NO:247), or TCCACTGTGCGACGAGCTGT (SEQ ID NO:249) can be used to detect a K15;R11 KIF5B-RET mutation; a probe comprising the sequence TGGGAAATAATGATGTAAA (SEQ ID NO:251), CTGTGGGAAATAATGATGTA (SEQ ID NO:253), GGAGGATCCAAAGTGGGAAT (SEQ ID NO:255), GGATCCAAAGTGGGAATT (SEQ ID NO:257), or ATGATGTAAAGGAGGATCC (SEQ ID NO:259) can be used to detect a K15;R12 KIF5B-RET mutation; a probe comprising the sequence CTTCGTATCTCTCAAGAGGAT (SEQ ID NO:481), GTATCTCTCAAGAGGATCCAA (SEQ ID NO:483), TTCGTATCTCTCAAGAG (SEQ ID NO:485), TCAAGAGGATCCAAA (SEQ ID NO:487), or TCTCTCAAGAGG (SEQ ID NO:489) can be used to detect a K16;R12 KIF5B-RET mutation; a probe comprising the sequence GTTAAAAAGGAGGATCCAA (SEQ ID NO:491), ACAAGAGTTAAAAAGGAGGA (SEQ ID NO:493), AAGAGTTAAAAAGGAGGATC (SEQ ID NO:495), AAAAGGAGGATCCAAAG (SEQ ID NO:497), or AAGGAGGATCCAAAGTG (SEQ ID NO:499) can be used to detect a K22;R12 KIF5B-RET mutation; and/or a probe comprising the sequence AAACAGGAGGATCCAAA (SEQ ID NO:501), AAGTGCACAAACAGGAGG (SEQ ID NO:503), GTGCACAAACAGGAGGATC (SEQ ID NO:505), CACAAACAGGAGGAT (SEQ ID NO:507), or AACAGGAGGATCCAAA (SEQ ID NO:509) can be used to detect a K23;R12 KIF5B-RET mutation. As described supra, in some embodiments, one or more probes of the present disclosure can comprise four, five, six, seven, or eight or more nucleotides (e.g., adenines or thymines) at its 5′ end. In certain embodiments, a probe comprising the sequence TTTTTTTTTTGTGGGAAATAATGATGTAAA (SEQ ID NO:242), TTTTTTTTTTCTGTGGGAAATAATGATGTA (SEQ ID NO:244), TTTTTTTTTTGATCCACTGTGCGACGAGCT (SEQ ID NO:246), TTTTTTTTTTTGATGTAAAGATCCACTGTG (SEQ ID NO:248), or TTTTTTTTTTTCCACTGTGCGACGAGCTGT (SEQ ID NO:250) can be used to detect a K15;R11 KIF5B-RET mutation. In certain embodiments, a probe comprising the sequence TTTTTTTTTTGGGAAATAATGATGTAAA (SEQ ID NO:252), TTTTTTTTTCTGTGGGAAATAATGATGTA (SEQ ID NO:254), TTTTTTTTTGGAGGATCCAAAGTGGGAAT (SEQ ID NO:256), TTTTTTTTTGGATCCAAAGTGGGAATT (SEQ ID NO: 258), or TTTTTTTTTATGATGTAAAGGAGGATCC (SEQ ID NO:260) can be used to detect a K15;R12 KIF5B-RET mutation. In certain embodiments, a probe comprising the sequence TTTTTTTTTCTTCGTATCTCTCAAGAGGAT (SEQ ID NO:482), TTTTTTTTTGTATCTCTCAAGAGGATCCAA (SEQ ID NO:484), TTTTTTTTTTTCGTATCTCTCAAGAG (SEQ ID NO:486), TTTTTTTTTTCAAGAGGATCCAAA (SEQ ID NO:488), or TTTTTTTTTTCTCTCAAGAGG (SEQ ID NO:490) can be used to detect a K16;R12 KIF5B-RET mutation. In certain embodiments, a probe comprising the sequence TTTTTTTTTGTTAAAAAGGAGGATCCAA (SEQ ID NO:492), TTTTTTTTACAAGAGTTAAAAAGGAGGA (SEQ ID NO:494), TTATTATTAAGAGTTAAAAAGGAGGATC (SEQ ID NO:811), TTTTTTTTAAAAGGAGGATCCAAAG (SEQ ID NO:498), or TTTTTTTTAAGGAGGATCCAAAGTG (SEQ ID NO:500) can be used to detect a K22;R12 KIF5B-RET mutation. In certain embodiments, a probe comprising the sequence TTTTTTTTAAACAGGAGGATCCAAA (SEQ ID NO:502), TTTTTATTAAGTGCACAAACAGGAGG (SEQ ID NO:504), TATTATTATGTGCACAAACAGGAGGATC (SEQ ID NO:506), TATTTTTTCACAAACAGGAGGAT (SEQ ID NO:508), or TTTTATTTAACAGGAGGATCCAAA (SEQ ID NO:510) can be used to detect a K23;R12 KIF5B-RET mutation. Probes comprising these sequences exclusive of the 5′ adenine and/or thymines are also contemplated.

In some embodiments, one or more probe(s) specific for an RNA mutation in an NTRK1 gene (e.g., resulting in a CD74-NTRK1 fusion gene) as described herein is/are used. For example, a probe comprising the sequence CAGGATCTGGGCCCAGACA (SEQ ID NO:261), GATCTGGGCCCAGACACTA (SEQ ID NO:263), CCAGACACTAACAGCACAT (SEQ ID NO:265), GGGCCCAGACACTAACAGC (SEQ ID NO:267), or CTAACAGCACATCTGGAGA (SEQ ID NO:269) can be used to detect a CD74-NTRK1 mutation. As described supra, in some embodiments, one or more probes of the present disclosure can comprise four, five, six, seven, or eight or more nucleotides (e.g., adenines or thymines) at its 5′ end. In certain embodiments, a probe comprising the sequence TTTTTTTTTTACAGGATCTGGGCCCAGACA (SEQ ID NO:262), TTTTTTTTTTAGATCTGGGCCCAGACACTA (SEQ ID NO:264), TTTTTTTTTTACCAGACACTAACAGCACAT (SEQ ID NO:266), TTTTTTTTTTAGGGCCCAGACACTAACAGC (SEQ ID NO:268), or TTTTTTTTTTACTAACAGCACATCTGGAGA (SEQ ID NO:270) can be used to detect a CD74-NTRK1 mutation.

In some embodiments, one or more probe(s) specific for an RNA mutation in a cMET gene (e.g., resulting in skipping of exon 14) as described herein is/are used. For example, a probe comprising the sequence AGAAAGCAAATTAAAGAT (SEQ ID NO:271), AGCAAATTAAAGATCAG (SEQ ID NO:273), AAATTAAAGATCAGTTTC (SEQ ID NO:275), AGATCAGTTTCCTAATTC (SEQ ID NO:277), or AAGATCAGTTTCCTAATT (SEQ ID NO:279) can be used to detect a cMET exon 14 skipping mutation. As described supra, in some embodiments, one or more probes of the present disclosure can comprise four, five, six, seven, or eight or more nucleotides (e.g., adenines or thymines) at its 5′ end. In certain embodiments, a probe comprising the sequence TTTTTTTTTTAGAAAGCAAATTAAAGAT (SEQ ID NO:272), TTTTTTTTTTAGCAAATTAAAGATCAG (SEQ ID NO:274), TTTTTTTTTTAAATTAAAGATCAGTTTC (SEQ ID NO:276), TTTTTTTTTTAGATCAGTTTCCTAATTC (SEQ ID NO:278), or TTTTTTTTTTAAGATCAGTTTCCTAATT (SEQ ID NO:280) can be used to detect a cMET exon 14 skipping mutation. Probes comprising these sequences exclusive of the 5′ adenine and/or thymines are also contemplated.

It is to be understood that the primers, blocking nucleic acids, and probes described above can be combined in any number or combination in the methods and kits of the present disclosure. Exemplary and non-limiting kits are described in greater detail infra.

Detection

In some embodiments, the methods of the present disclosure include detecting presence or absence of hybridization of amplified DNA with a probe of the present disclosure. Hybridization between the amplified DNA and one of the probes indicates the presence of the DNA or RNA mutation corresponding to the probe in the amplified DNA. Exemplary hybridization conditions and detection techniques are described and exemplified herein. In some embodiments, hybridization is performed using 5×SSPE buffer.

In some embodiments, the amplified DNA or cDNA is labeled with a detection reagent, and hybridization is measured by signal of the detection reagent associated with the microcarrier, e.g., after a washing step to reduce or eliminate non-specific binding. In some embodiments, a primer pair of the present disclosure comprises one or both primers coupled to the detection reagent. Thus, the amplified DNA is labeled with the detection reagent after PCR amplification using the labeled primer(s). In some embodiments, the detection reagent can be fluorescence-based including, but not limited to, phycoerythrin (PE), blue fluorescent protein, green fluorescent protein, yellow fluorescent protein, cyan fluorescent protein, and derivatives thereof. In other embodiments, the detection reagent can be radioisotope based, including, but not limited to, molecules labeled with ³²P, ³³P, ²²Na, ³⁶Cl, ²H, ³H, ³¹S, and ¹²³I. In other embodiments, the detection reagent is light-based including, but not limited to, luciferase (e.g. chemiluminescence-based), horseradish peroxidase, alkaline phosphatase and derivatives thereof. In some embodiments, the amplified DNA can be labeled with the detection reagent prior to contact with the microcarrier composition. In some embodiments, the detection reagent emits a signal when in close proximity to the probe, e.g., as with Forster resonance energy transfer (FRET). A variety of nucleic acid labeling techniques are known in the art; see, e.g., Gibriel, A. A. Y. (2012) Briefings in Functional Genomics 11:311-8.

In some embodiments, the detection reagent can be a fluorescent detection reagent. In some embodiments, detecting the presence or absence of hybridization of amplified DNA with a probe is performed by fluorescence microscopy (e.g., a fluorescent microscope or plate reader). In some embodiments, the detection reagent can be colorimetric based. In some embodiments, the detection reagent can be luminescence based. In some embodiments, detecting the presence or absence of hybridization of amplified DNA with a probe is performed by luminescence microscopy (e.g., a luminescent microscope or plate reader).

In some embodiments, the detection reagent comprises a tag or other moiety that can be detected by the addition of a secondary reagent conjugated to a signal-emitting entity (e.g., as described above). For example, in some embodiments, the detection reagent comprises biotin (e.g., a primer such as the reverse or antisense primer may be labeled at the 5′ end with biotin). Thus, in some embodiments, the detecting presence or absence of hybridization can include, after hybridization and optional washing, contacting microcarrier(s) with a secondary reagent conjugated to a signal-emitting entity and detecting a signal from the signal-emitting entity in association with the microcarrier(s) (e.g., after optional washing). For example, if the detection reagent comprises biotin, the microcarriers can be contacted with streptavidin conjugated to a signal-emitting entity such as phycoerythrin (PE), and signal from the signal-emitting entity can be detected.

Depending upon the particular detection reagent, a variety of techniques may be used to detect hybridization of the amplified DNA to a probe. For example, if the detection reagent comprises a fluorescent detection reagent, detecting the presence or absence of hybridization of the amplified DNA can comprise fluorescence imaging of the fluorescent detection reagent.

In some embodiments, detecting may include one or more washing steps, e.g., to reduce contaminants, remove any substances non-specifically bound to the probe, DNA, and/or microcarrier surface, and so forth. In some embodiments, a magnetic separation step may be used to wash a microcarrier containing a magnetic layer or material of the present disclosure. In other embodiments, other separation steps known in the art may be used.

In some embodiments, the methods of the present disclosure comprise detecting the presence or absence of hybridization of amplified DNA with a total of between about 1 and about 1000 microcarriers per probe per assay. In some embodiments, the methods of the present disclosure comprise detecting the presence or absence of hybridization of amplified DNA with a total of between about 1 and about 1000 microcarriers per probe per well of an assay plate. In some embodiments, the methods of the present disclosure comprise detecting the presence or absence of hybridization of amplified DNA with at least about 50 microcarriers per probe per assay. In some embodiments, the methods of the present disclosure comprise detecting the presence or absence of hybridization of amplified DNA with at least about 50 microcarriers per probe per well of an assay plate. In some embodiments, a microcarrier of the present disclosure comprises the probe coupled thereto at a concentration of 1 μM.

In some embodiments, the methods of the present disclosure comprise detecting the identifier of an encoded microcarrier. For example, in some embodiments, an image of the identifier of an encoded microcarrier can be obtained and decoded to identify the microcarrier and its corresponding probe. In some embodiments, the identifier detection step(s) may occur after the hybridization detection step(s). In other embodiments, the identifier detection step(s) may occur before the hybridization detection step(s). In still other embodiments, the identifier detection step(s) may occur simultaneously with the hybridization detection step(s). In some embodiments, after the hybridization detection step(s) and the identifier detection step(s), the methods of the present disclosure further comprise correlating the detected identifiers of the microcarriers with the detected presence or absence of hybridization of the amplified DNA to the corresponding probes of the microcarriers.

A variety of microcarrier coding schemes are described herein. In some embodiments, detecting the identifier of an encoded microcarrier comprises imaging a digital barcode of the microcarrier. In one embodiment, the coded microcarrier comprises a body having a series of alternating light transmissive and opaque sections, with relative widths bar code image (e.g., a series of narrow slits representing a “0” code and wide slits representing a “1” code, or vice versa). When the microcarrier is illuminated with a light beam, based on the either the “total intensity” of the transmission peak or the “bandwidth” of the transmission peak from the slit, the digital barcode either 0 or 1 can be determined by a line scan camera, a frame grabber, and a digital signal processor. In one embodiment, the bar code pattern with a series of narrow and wide bands provides an unambiguous signal and differentiation for 0's and 1's. The position of the slits on the pallet will determine which of the bits is the least significant (LSB) and most significant bit (MSB). The LSB will be placed closer to the edge of the pallet to distinguish it from the MSB at the other, longer end.

In some embodiments, detecting the identifier of an encoded microcarrier comprises imaging the identifier of the microcarrier, e.g., by bright field imaging of the identifier, such as with an analog code of the present disclosure.

A variety of decoding techniques are contemplated. In some embodiments, an identifier is detected using analog shape recognition to identify the identifier (e.g., an analog-encoded identifier). Conceptually, this decoding may involve, for example, imaging the analog code of each microcarrier (e.g., in a solution or sample), comparing each image against a library of analog codes, and matching each image to an image from the library, thus positively identifying the code. Optionally, as described herein, when using microcarriers that include an orientation indicator (e.g., an asymmetry), the decoding may further include a step of rotating each image to align with a particular orientation (based in part, e.g., on the orientation indicator). For example, if the orientation indicator includes a gap, the image could be rotated until the gap reaches a predetermined position or orientation (e.g., a 0° position of the image).

Various shape recognition software, tools, and methods are known in the art. Examples of such APIs and tools include without limitation Microsoftk Research FaceSDK, OpenBR, Face and Scene Recognition from ReKognition, Betaface API, and various ImageJ plugins. In some embodiments, the analog shape recognition may include without limitation image processing steps such as foreground extraction, shape detection, thresholding (e.g., automated or manual image thresholding), and the like.

It will be appreciated by one of skill in the art that the methods and microcarriers described herein may be adapted for various imaging devices, including without limitation a microscope, plate reader, and the like. In some embodiments, decoding an identifier (e.g., an analog code) can include illuminating the microcarrier by passing light through a substantially transparent portion (e.g., substantially transparent polymer layer(s)) of the microcarrier and/or the surrounding solution). The light may then fail to pass through, or pass through with a lower intensity or other appreciable difference, the substantially non-transparent portions (e.g., substantially non-transparent layer(s)) of the microcarrier to generate an analog-coded light pattern corresponding to the identifier. That is to say, the pattern of imaged light may correspond to the pattern of substantially transparent/substantially non-transparent areas of the microcarrier, thus producing an image of the analog code identifier. This imaging may include steps including without limitation capturing the image, thresholding the image, and any other image processing step desired to achieve more accurate, precise, or robust imaging of the identifier.

Any type of light microscopy may be used for the methods of the present disclosure, including without limitation one or more of bright field, dark field, phase contrast, differential interference contrast (DIC), Nomarski interference contrast (NIC), Nomarski, Hoffman modulation contrast (HMC), or fluorescence microscopy. In certain embodiments, the identifiers may be decoded using bright field microscopy, and hybridization may be detected using fluorescence microscopy.

In some embodiments, decoding the identifiers may further include using analog shape recognition to match an analog-coded image with an analog code. In some embodiments, an image may be matched with an analog code (e.g., an image file from a library of image files, with each image file corresponding to a unique two-dimensional shape/analog code) within a predetermined threshold, e.g., that tolerates a predetermined amount of deviation or mismatch between the image and the exemplar analog code image. Such a threshold may be empirically determined and may naturally be based on the particular type of two-dimensional shapes used for the analog codes and the extent of variation among the set of potential two-dimensional shapes.

Exemplary and non-limiting descriptions of microcarriers, and optional aspects thereof, suitable for use in the methods of the present disclosure are provided infra.

### IV. Encoded Microcarriers

Provided herein are encoded microcarriers suitable for analyte detection, e.g., multiplex analyte detection. Multiple configurations for encoded microcarriers are contemplated, described, and exemplified herein. As used herein, an “encoded” microcarrier may refer to a microcarrier with an identifier that corresponds to the identity of a probe coupled thereto. This enables the data of an assay using the microcarrier to be associated with the identity of the probe, allowing for the use of multiple microcarriers in a single multiplex assay, since the results of any individual microcarrier can be correlated with the identity of its probe. Exemplary types of identifiers, including digital barcodes and analog codes, are described infra. Any of the microcarriers (or configurations or features thereof) described in International Publication No. WO2016198954 may find use in the methods and kits described herein.

In some aspects, the methods and kits of the present disclosure use digital barcode identifiers. For example, in some embodiments, encoded microcarriers comprise: a first photopolymer layer: a second photopolymer layer; and an intermediate layer between the first layer and the second layer. In some embodiments, the intermediate layer has an encoded pattern representing the identifier defined thereon, wherein the intermediate layer is partially substantially transmissive and partially substantially opaque to light, representing a code corresponding to the microcarrier, wherein the outermost surface of the microcarrier comprises a photoresist photopolymer, and said photoresist photopolymer is functionalized with the probe specific for the DNA mutation, and wherein said microcarrier has about the same density as water. Exemplary microcarrier descriptions may be found, e.g., in U.S. Pat. Nos. 7,858,307; 7,871,770; 8,148,139; 8,232,092; and 9,255,922; as well as US PG Pub. Nos. US2009/201504, 2011/0007955, and 2012/0088691.

In one embodiment, a digitally encoded microcarrier of the present disclosure comprises a body having a series of alternating light transmissive and opaque sections, with relative positions, widths and/or spacing resembling a 1D or 2D bar code image (e.g., a series of narrow slits (e.g., about 1 to 5 microns in width) representing a “0” code and wide slits (e.g., about 1 to 10 microns in width) representing a “1” code, or vice versa, to form a binary code). In one embodiment, the size of the microcarrier is sized and configured to be 150×50×10 μm, or proportionally smaller, and a slit width of about 2.5 μm. Each digital barcode on such a microcarrier can consist of up to 14 slits (or bits), allowing 16,384 unique codes. In one embodiment, the body of the coded microcarrier may be configured to have at least two orthogonal cross sections that are different in relative geometry and/or size. Further, the geometry of the cross sections may be symmetrical or non-symmetrical, and/or regular or irregular shape. In one embodiment, the longest orthogonal axis of the coded microcarrier is less than 1 mm. In one embodiment, the coded microcarrier is provided with a reflective thin film, (e.g., plating or coating the coded microcarrier with a metal thin film, or providing an intermediate layer of metal thin film) to improve contrast and optical efficiency for image recognition for decoding. One alternate embodiment may include a metal layer as a layer sandwiched between two polymeric layers, by appropriately modifying the above described process. With this embodiment, surface condition could be made the same for both exposed planar surfaces of the microcarrier, to provide similar surface coating and immobilization conditions. Another embodiment is to coat the microcarrier with polymer or functional molecules, such as a probe of the present disclosure; therefore, the whole microcarrier has the same condition for molecular immobilization.

In some aspects, the methods and kits of the present disclosure use analog code identifiers. For example, in some embodiments, the methods and kits of the present disclosure use encoded microcarriers that comprise: a substantially transparent polymer layer having a first surface and a second surface, the first and the second surfaces being parallel to each other; a substantially non-transparent layer, wherein the substantially non-transparent polymer layer is affixed to the first surface of the substantially transparent polymer layer and encloses a center portion of the substantially transparent polymer layer, and wherein the substantially non-transparent polymer layer comprises a two-dimensional shape representing an analog code; and a probe of the present disclosure specific for a DNA or RNA mutation, wherein the probe is coupled to at least one of the first surface and the second surface of the substantially transparent polymer layer in at least the center portion of the substantially transparent polymer layer. The analog code represents the identifier. Thus, the microcarrier contains at least two layers: one of which is substantially transparent, and the other of which is a substantially non-transparent, two-dimensional shape that represents an analog code identifier.

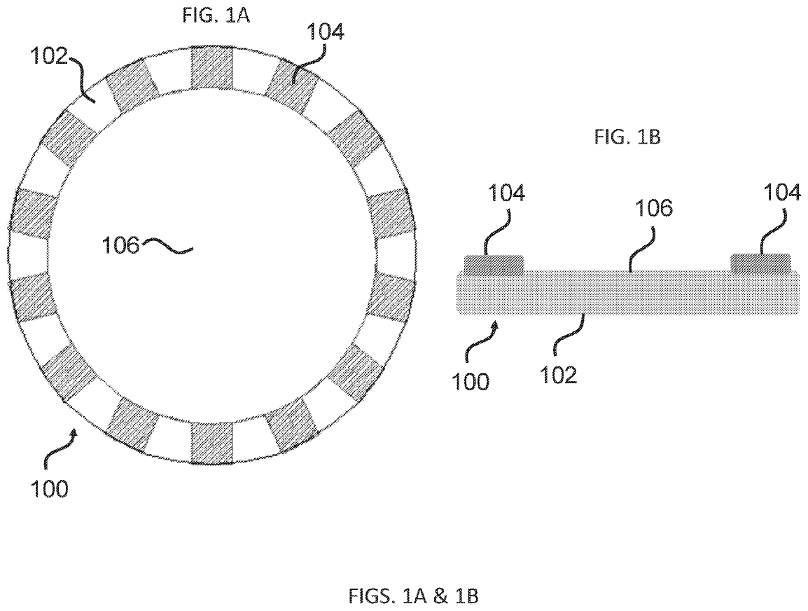

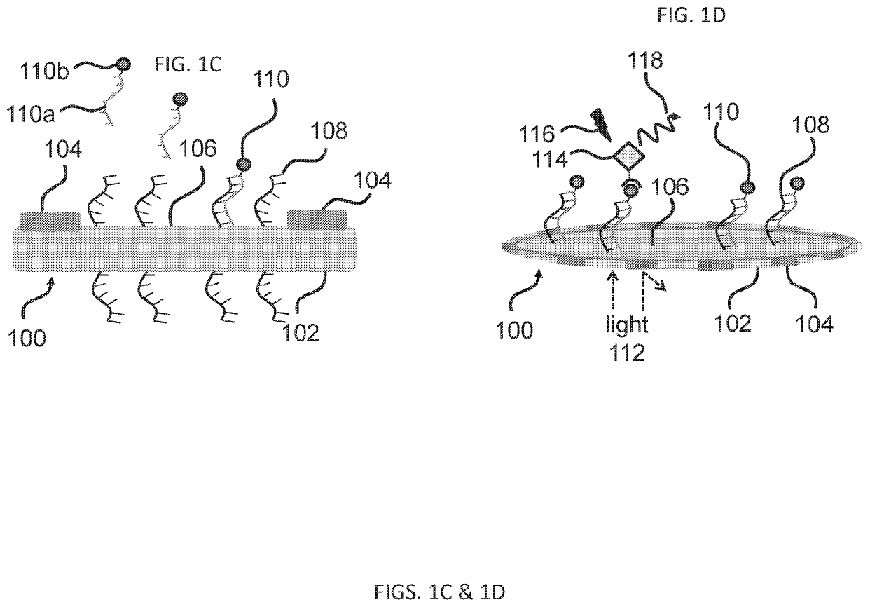

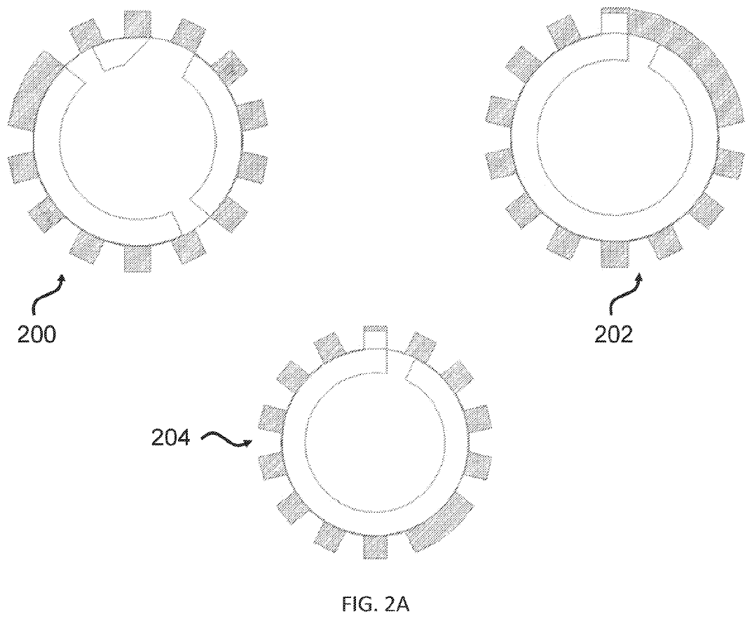

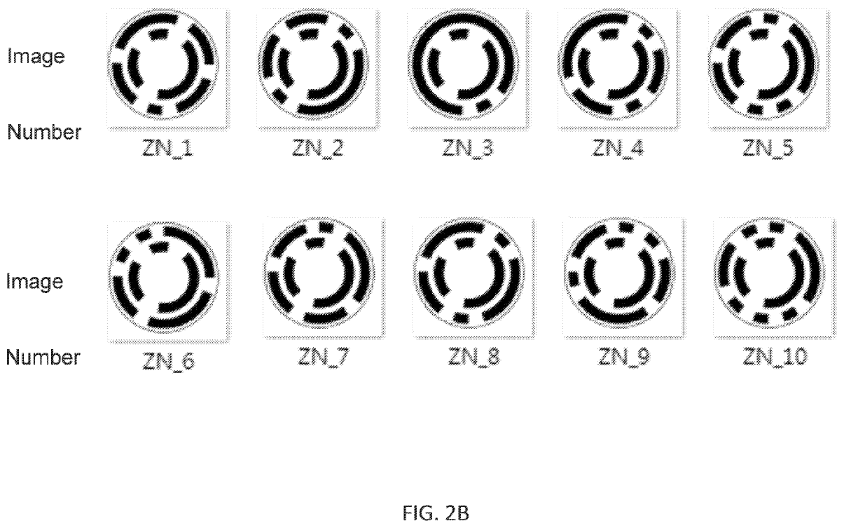

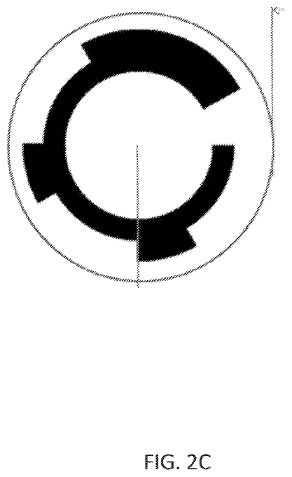

Advantageously, these microcarriers may employ a variety of two-dimensional shapes while still retaining a uniform overall form (e.g., the perimeter of the substantially transparent polymer layer) for uniformity of aspects including, for example, overall dimensions, physical properties, and/or behavior in solution. This is advantageous, for example, in allowing greater uniformity between different species of microcarriers (i.e., each has the same perimeter shape provided by the transparent polymer layer). Examples of this type of microcarrier and aspects thereof are illustrated in FIGS. 1A-2C.

FIGS. 1A & 1B show two views of exemplary microcarrier 100. Microcarrier 100 is a circular disc of approximately 50 μm in diameter and 10 μm in thickness. FIG. 1A provides a view of microcarrier 100 looking at a circular face of the disc, while FIG. 1B shows a side view of microcarrier 100 orthogonal to the surface shown in FIG. 1A. Two components of microcarrier 100 are shown. First, substantially transparent polymer layer 102 provides the body of the microcarrier. Layer 102 may be produced, e.g., using a polymer such as SU-8, as described herein.

Substantially non-transparent polymer layer 104 is affixed to a surface of layer 102. While the cross-section of microcarrier 100 shown in FIG. 1B shows a discontinuous view of layer 104, the view shown in FIG. 1A illustrates that layer 104 is shaped like a circular gear with a plurality of teeth. The shape, number, size, and spacing of these gear teeth constitutes a two-dimensional shape, and one or more of these aspects of the gear teeth may be modified in order to produce multiple two-dimensional shapes for analog encoding. Advantageously, the outside edge of layer 104's gear teeth fit within the perimeter of layer 102. This allows for a variety of analog codes, each representing a unique identifier for one species of microcarrier, while maintaining a uniform overall shape across multiple species of microcarrier. Stated another way, each microcarrier species within a population of multiple species may have a different two-dimensional gear shape (i.e., analog code), but each microcarrier will have the same perimeter, leading to greater uniformity of physical properties (e.g., size, shape, behavior in solution, and the like). Layer 104 may be produced, e.g., using a polymer such as SU-8 mixed with a dye, or using a black matrix resist, as described herein. This is merely an exemplary shape; other shapes are described above and shown, e.g., in FIGS. 2A-2C.

Layer 104 surrounds center portion 106 of layer 102. A probe is coupled to at least center portion 106 on one or both surfaces (i.e., upper/lower surfaces) of layer 102. Advantageously, this allows center portion 106 to be imaged without any potential for interference resulting from layer 104.

FIGS. 1C & ID show an exemplary assay using microcarrier 100 for analyte detection. FIG. 1C shows that microcarrier 100 may include probe 108 coupled to one or more surfaces in at least center portion 106. Microcarrier 100 is contacted with a solution containing amplified DNA 110, which has been denatured prior to contacting microcarrier 100 and hybridizes to probe 108. As described above, amplified DNA 110 is coupled to a detection reagent. In this example, the detection reagent is biotin (e.g., resulting from amplification of DNA using a biotin-labeled primer). Thus, amplified DNA 110 includes DNA 110a (e.g., comprising the locus of a mutation described herein) and biotin 110b. FIG. 1C illustrates a single microcarrier species (i.e., microcarrier 100), which captures amplified DNA 110, but in a multiplex assay multiple microcarrier species are used, each species having a particular probe that recognizes a specific DNA mutation.

FIG. 1D illustrates an exemplary process for “reading” microcarrier 100. This process includes two steps that may be accomplished simultaneously or separately. First, the hybridization of amplified DNA 110 by probe 108 is detected. In the example shown in FIG. 1D, secondary detection reagent 114 (e.g., streptavidin conjugated to PE) binds to amplified DNA 110 via a biotin:streptavidin interaction. Amplified DNA not hybridized to a probe coupled to microcarrier 100 may have been washed off prior to detection, such that only DNA bound to microcarrier 100 is detected. The PE moiety of secondary detection reagent 114 emits light 118 (e.g., a photon) when excited by light 116 at a wavelength within the excitation spectrum of PE. Light 118 may be detected by any suitable detection means, such as a fluorescence microscope, plate reader, and the like.

In addition, microcarrier 100 is read for its unique identifier. In the example shown in FIG. 1D, light 112 is used to illuminate the field containing microcarrier 100 (in some embodiments, light 112 may have a different wavelength than lights 116 and 118). When light 112 illuminates the field containing microcarrier 100, it passes through substantially transparent polymer layer 102 but is blocked by substantially non-transparent polymer layer 104, as shown in FIG. 1D. This generates a light pattern that can be imaged, for example, by light microscopy (e.g., using differential interference contrast, or DIC, microscopy). This light pattern is based on the two-dimensional shape (i.e., analog code) of microcarrier 100. Standard image recognition techniques may be used to decode the analog code represented by the image of microcarrier 100.

As described in greater detail below, the analyte detection and identifier imaging steps may occur in any order, or simultaneously. Advantageously, both detection steps shown in FIG. 1D may be accomplished on one imaging device. As one example, a microscope capable of both fluorescence and light (e.g., bright field) microscopy may be used to quantify the amount of amplified DNA 110 bound to microcarrier 100 (e.g., as detected by detection reagent 114) and image the analog code created by layers 102 and 104. This allows for a more efficient assay process with fewer equipment requirements.

In some embodiments, the microcarrier further includes a magnetic, substantially non-transparent layer affixed to a surface of the substantially transparent polymer layer that encloses the center portion of the substantially transparent polymer layer. In some embodiments, the magnetic, substantially non-transparent layer is between the substantially non-transparent polymer layer and the center portion of the substantially transparent polymer layer.

In some embodiments, the microcarrier further includes a second substantially transparent polymer layer aligned with and affixed to the first substantially transparent polymer layer. In some embodiments, the first and second substantially transparent polymer layers each have a center portion, and the center portions of both the first and second substantially transparent polymer layers are aligned. In some embodiments, the microcarrier further includes a magnetic, substantially non-transparent layer that encloses the center portions of both the first and second substantially transparent polymer layers. In some embodiments, the magnetic, substantially non-transparent layer is affixed between the first and second substantially transparent polymer layers. In some embodiments, the magnetic, substantially non-transparent layer is between the substantially non-transparent polymer layer and the center portions of both the first and second substantially transparent polymer layers.

In some embodiments, the microcarrier further includes an orientation indicator for orienting the analog code of the substantially non-transparent polymer layer. Any feature of the microcarrier that is visible and/or detectable by imaging (e.g., a form of microscopic or other imaging described herein) and/or by image recognition software may serve as an orientation indicator. An orientation indicator may serve as a point of reference, e.g., for an image recognition algorithm, to orient the image of an analog code in a uniform orientation (i.e., the shape of the substantially non-transparent polymer layer). Advantageously, this simplifies image recognition, as the algorithm would only need to compare the image of a particular analog code against a library of analog codes in the same orientation, and not against a library including all analog codes in all possible orientations. In some embodiments, the orientation indicator comprises an asymmetry of the outline of the substantially non-transparent polymer layer. For example, the orientation indicator may comprise a visible feature, such as an asymmetry, of the outline of the microcarrier (e.g., as illustrated by the discontinuity in the ring of the code shown in FIG. 2C).

Any of the microcarriers described herein may include one or more of the features, elements, or aspects described below. In addition, one or more of the features, elements, or aspects described below may adopt different characteristics depending on the embodiment of the microcarrier, e.g., as described above.

In some embodiments, a polymer of the present disclosure comprises an epoxy-based polymer. Suitable epoxy-based polymers for fabrication of the compositions described herein include, but are not limited to, the EPON™ family of epoxy resins provided by Hexion Specialty Chemicals, Inc. (Columbus, Ohio) and any number of epoxy resins provided by The Dow Chemical Company (Midland, Mich.). Many examples of suitable polymers are commonly known in the art, including without limitation SU-8, EPON 1002F, EPON 165/154, and a poly(methyl methacrylate)/poly(acrylic acid) block copolymer (PMMA-co-PAA). For additional polymers, see, for example, Warad, IC Packaging: Package Construction Analysis in Ultra Small IC Packaging, LAP LAMBERT Academic Publishing (2010); The Electronic Packaging Handbook, CRC Press (Blackwell, ed.), (2000); and Pecht et al., Electronic Packaging Materials and Their Properties, CCR Press, 1^{st} ed., (1998). These types of materials have the advantage of not swelling in aqueous environments which ensures that uniform microcarrier size and shape are maintained within the population of microcarriers. In some embodiments, the substantially transparent polymer is a photoresist polymer. In some embodiments, the epoxy-based polymer is an epoxy-based, negative-tone, near-UV photoresist. In some embodiments, the epoxy-based polymer is SU-8.

In some embodiments, the substantially non-transparent polymer is a polymer described herein (e.g., SU-8) mixed with one or more non-transparent or colored dye(s). In other embodiments, the substantially non-transparent polymer is a black matrix resist. Any black matrix resist known in the art may be used; see, e.g., U.S. Pat. No. 8,610,848 for exemplary black matrix resists and methods related thereto. In some embodiments, the black matrix resist may be a photoresist colored with a black pigment, e.g., as patterned on the color filter of an LCD as part of a black matrix. Black matrix resists may include without limitation those sold by Toppan Printing Co. (Tokyo). Tokyo OHKA Kogyo (Kawasaki), and Daxin Materials Corp. (Taichung City, Taiwan).

As described above, a microcarrier of the present disclosure may further include a magnetic layer, which may adopt a variety of shapes as described herein. In some embodiments, the magnetic layer may be a substantially non-transparent layer. In some embodiments, the magnetic layer may comprise a magnetic material. A magnetic layer of the present disclosure may be made of any suitable magnetic material, such as a material with paramagnetic, ferromagnetic, or ferrimagnetic properties. Examples of magnetic materials include without limitation iron, nickel, cobalt, and some rare earth metals (e.g., gadolinium, dysprosium, neodymium, and so forth), as well as alloys thereof. In some embodiments, the magnetic material comprises nickel, including without limitation elemental nickel and magnetic nickel alloys such as alnico and permalloy. The inclusion of a magnetic layer in a microcarrier of the present disclosure may be advantageous, e.g., in facilitating magnetic separation, which may be useful for washing, collecting, and otherwise manipulating one or more microcarriers.

In some embodiments, a microcarrier of the present disclosure may be encoded with a substantially non-transparent layer that constitutes a two-dimensional shape. For example, as described above, the two-dimensional shape may constitute the shape of a substantially non-transparent layer that contrasts with a substantially transparent layer of the microcarrier, or it may constitute the shape of the microcarrier itself (e.g., the perimeter). That is to say, the code is the shape of the substantially non-transparent layer itself (e.g., rather than a code generated by a fluorescent or other visible moiety on the surface of a layer of the microcarrier). Any two-dimensional shape that can encompass a plurality of resolvable and distinctive varieties may be used. In some embodiments, the two-dimensional shape comprises one or more of linear, circular, elliptical, rectangular, quadrilateral, or higher polygonal aspects, elements, and/or shapes. In some embodiments, the two-dimensional shape may be produced using a magnetic, substantially non-transparent layer of the present disclosure. In some embodiments, the analog code comprises one or more overlapping or partially overlapping arc elements forming a continuous or discontinuous ring (e.g., surrounding a center portion of the microcarrier).

FIG. 2A illustrates three exemplary embodiments of an analog coding scheme: microcarriers 200, 202, and 204. Importantly, as described above, more complex encoding schemes are available using analog image recognition, thereby greatly expanding the number of potential unique codes. In some embodiments, the two-dimensional shape of the substantially non-transparent polymer layer comprises one or more rings enclosing the center portion of the substantially transparent polymer layer. In some embodiments, at least one of the one or more rings comprises a discontinuity. Exemplary and non-limiting two-dimensional shapes formed using one or more rings (e.g., two rings) having varying numbers and configurations of discontinuities are illustrated in FIG. 2B. FIG. 2B illustrates 10 exemplary embodiments of the cod, inter alia, in terms of number of shapes (e.g., two distinct shapes in code ZN_3, as compared to seven distinct shapes in code ZN_10) and/or size of shapes (e.g., large, small, and intermediate-sized shapes in code ZN_2).

In some embodiments, the analog code comprises one or more overlapping or partially overlapping arc elements forming a continuous or discontinuous ring (e.g., surrounding a center portion of the microcarrier). The two-dimensional shape is decoded by imaging the microcarrier (e.g., by light microscopy), such that an image of the code is formed by the pattern generated by light passed through the substantially transparent magnetic polymer layer and light blocked from passing through the substantially non-transparent layer. A non-limiting example of a two-dimensional shape made of overlapping arc elements that form a discontinuous ring is illustrated in FIG. 2C.

In some embodiments, the microcarrier is less than about 200 μm in diameter. For example, in some embodiments, the diameter of the microcarrier is less than about 200 μm, less than about 180 μm, less than about 160 μm, less than about 140 μm, less than about 120 μm, less than about 100 μm, less than about 80 μm, less than about 60 μm, less than about 40 μm, or less than about 20 μm.

In some embodiments, the diameter of the microcarrier is about 180 μm, about 160 m, about 140 μm, about 120 μm, about 100 μm, about 90 μm, about 80 μm, about 70 m, about 60 μm, about 50 μm, about 40 μm, about 30 μm, about 20 μm, or about 10 m. In certain embodiments, the microcarrier is about 60 μm in diameter.

In some embodiments, the microcarrier is less than about 50 μm in thickness. For example, in some embodiments, the thickness of the microcarrier is less than about 70 μm, about 60 μm, about 50 μm, about 40 μm, about 30 μm, less than about 25 μm, less than about 20 μm, less than about 15 μm, less than about 10 μm, or less than about 5 μm. In some embodiments, the thickness of the microcarrier is less than about any of the following thicknesses (in μm): 70, 65, 60, 55, 50, 45, 40, 35, 30, 25, 20, 15, 10, 9, 8, 7, 6, 5, 4, 3, or 2. In some embodiments, the thickness of the microcarrier is greater than about any of the following thicknesses (in μm): 1, 2, 3, 4, 5, 6, 7, 8, 9, 10, 15, 20, 25, 30, 35, 40, 45, 50, 55, 60, or 65. That is, the thickness of the microcarrier may be any of a range of thicknesses (in μm) having an upper limit of 70, 65, 60, 55, 50, 45, 40, 35, 30, 25, 20, 15, 10, 9, 8, 7, 6, 5, 4, 3, or 2 and an independently selected lower limit of 1, 2, 3, 4, 5, 6, 7, 8, 9, 10, 15, 20, 25, 30, 35, 40, 45, 50, 55, 60, or 65, wherein the lower limit is less than the upper limit.

In some embodiments, the thickness of the microcarrier is about 50 μm, about 45 μm, about 40 μm, about 35 μm, about 30 μm, about 25 μm, about 20 μm, about 19 μm, about 18 μm, about 17 μm, about 16 μm, about 15 μm, about 14 μm, about 13 μm, about 12 μm, about 11 μm, about 10 μm, about 9 μm, about 8 μm, about 7 μm, about 6 μm, about 5 μm, about 4 μm, about 3 μm, about 2 μm, or about 1 μm. In certain embodiments, the microcarrier is about 10 m in thickness.

A variety of techniques may be used to couple a probe of the present disclosure to an encoded microcarrier. In some embodiments, the probe is coupled to a surface of the microcarrier (in some embodiments, in at least a center portion of the microcarrier surface). In some embodiments, the probe may be coupled to one or both of a first or a second surface of the polymer layer. In some embodiments, the polymer comprises an epoxy-based polymer or otherwise contains an epoxide group.

In some embodiments, the probe can be chemically attached to the microcarrier. In other embodiments, the probe can be physically absorbed to the surface of the microcarrier. In some embodiments, the attachment linkage between the probe and the microcarrier surface can be a covalent bond. In other embodiments, the attachment linkage between the probe and the microcarrier surface can be a non-covalent bond including, but not limited to, a salt bridge or other ionic bond, one or more hydrogen bonds, hydrophobic interactions, Van der Waals force, London dispersion force, a mechanical bond, one or more halogen bonds, aurophilicity, intercalation, or stacking.

In some embodiments, coupling the probe involves reacting the polymer with a photoacid generator and light to generate a cross-linked polymer. In some embodiments, the light is of a wavelength that activates the photoacid generator, e.g., UV or near-UV light. Photoacid generators are commercially available from Sigma-Aldrich (St. Louis) and BASF (Ludwigshafen). Any suitable photoacid generator known in the art may be used, including without limitation triphenyl or triaryl sulfonium hexafluoroantimonate; triarylsulfonium hexafluorophosphate; triphenylsulfonium perfluoro-1-butanesulfonate; triphenylsulfonium triflate; Tris(4-tert-butylphenyl)sulfonium perfluoro-1-butanesulfonate or triflate; Bis(4-tert-butylphenyl)iodonium-containing photoacid generators such as Bis(4-tert-butylphenyl)iodonium perfluoro-1-butanesulfonate, p-toluenesulfonate, and triflate; Boc-methoxyphenyldiphenylsulfonium triflate; (tert-Butoxycarbonylmethoxvnaphthyl)-diphenylsulfonium triflate; (4-tert-Butylphenyl)diphenylsulfonium triflate; diphenyliodonium hexafluorophosphate, nitrate, perfluoro-1-butanesulfonate, triflate, or p-toluenesulfonate; (4-fluorophenyl)diphenylsulfonium triflate; N-hydroxynaphthalimide triflate; N-hydroxy-5-norbornene-2,3-dicarboximide perfluoro-1-butanesulfonate; (4-iodophenyl)diphenylsulfonium Inflate; (4-methoxyphenyl)diphenylsulfonium triflate; 2-(4-methoxystyryl)-4,6-bis(trichloromethyl)-1,3,5-triazine; (4-methylphenyl)diphenylsulfonium triflate; (4-methylthiophenyl)methyl phenyl sulfonium triflate; (4-phenoxyphenyl)diphenylsulfonium triflate; (4-phenylthiophenyl)diphenylsulfonium triflate; or any of the photoacid generators described in product-finder.basf.com/group/corporate/product-finder/de/literature-document:/Brand+Irgacure-Brochure--Photoacid+Generator+Selection+Guide-English.pdf. In some embodiments, the photoacid generator is a sulfonium-containing photoacid generator.

In some embodiments, coupling the probe involves reacting an epoxide of the cross-linked polymer with a functional group such as an amine, carboxyl, thiol, or the like. Alternatively, the epoxy group on the surface can be oxidized to hydroxyl group, which is subsequently used as initiation sites for graft polymerization of water soluble polymers such as poly(acrylic acid). The carboxyl groups in poly(acrylic acid) are then used to form covalent bonds with amino or hydroxyl groups in capture agents. For example, in certain embodiments, the carboxyl groups in poly(acrylic acid) are used to form covalent bonds with amino groups in the probe.

In some embodiments, coupling the probe involves reacting an epoxide of the cross-linked polymer with a compound that contains an amine and a carboxyl. In some embodiments, the amine of the compound reacts with the epoxide to form a compound-coupled, cross-linked polymer. Without wishing to be bound to theory, it is thought that the probe may be coupled to the polymer before the polymer is cross-linked; however, this may reduce the uniformity of the resulting surface. Any compound with a primary amine and a carboxyl group may be used. Compounds may include without limitation glycine, amino undecanoic acid, amino caproic acid, acrylic acid, 2-carboxyethyl acrylic acid, 4-vinylbenzoic acid, 3-acrylamido-3-methyl-1-butanoic acid, glycidyl methacrylate, and the like. In some embodiments, the carboxyl of the compound-coupled, cross-linked polymer reacts with an amine (e.g., a primary amine) of the probe to couple the capture agent to the substantially transparent polymer.

Methods for making any of the encoded microcarriers described herein are described in greater detail in International Publication No. WO2016198954.

### V. Kits

Further provided herein are kits or articles of manufacture containing a plurality of microcarriers of the present disclosure. These kits or articles of manufacture may find use, inter alia, in conducting a multiplex assay, such as the exemplary multiplex assays described herein (see, e.g., section III above). Any of the microcarriers described herein (see, e.g., section IV above) may find use in a kit or article of manufacture of the present disclosure

In some embodiments, a kit or article of manufacture of the present disclosure comprises at least seven encoded microcarriers. In some embodiments, each of the seven encoded microcarriers comprises (i) a probe, specific for a DNA mutation in the KRAS, PIK3CA, BRAF, EGFR, AKT1, MEK1, or HER2 gene, coupled to the microcarrier; and (ii) an identifier corresponding to the probe. In some embodiments, the kit comprises at least one microcarrier comprising a probe specific for a DNA mutation in the KRAS gene, at least one microcarrier comprising a probe specific for a DNA mutation in the PIK3CA gene, at least one microcarrier comprising a probe specific for a DNA mutation in the BRAF gene, at least one microcarrier comprising a probe specific for a DNA mutation in the EGFR gene, at least one microcarrier comprising a probe specific for a DNA mutation in the AKT1 gene, at least one microcarrier comprising a probe specific for a DNA mutation in the MEK1 gene, and at least one microcarrier comprising a probe specific for a DNA mutation in the HER2 gene. That is to say, each of the KRAS, NRAS, PIK3CA, BRAF, EGFR, AKT1, MEK1, and HER2 genes is represented in the kit by a microcarrier with a probe specific for a mutation in the gene. Exemplary KRAS, NRAS, PIK3CA, BRAF, EGFR, AKT1, MEK1, and HER2 genes and mutations are described supra. In some embodiments, the KRAS, NRAS, PIK3CA, BRAF, EGFR, AKT1, MEK1, and HER2 genes are human genes. In some embodiments, the kit comprises microcarriers with probes suitable for detecting each of the following mutations (e.g., with at least one microcarrier+probe species for each mutation in the kit): DNA mutations encoding G12D, and G12V mutated KRAS proteins; DNA mutations encoding E542K, E545K, and H1047R mutated PIK3CA proteins; a DNA mutation encoding a V600E mutated BRAF protein; DNA mutations encoding G719A, E746_A750del, T790M, C797S (T>A and/or G>C), S768I, V769_D770insASV, H773_V774insH, D770_N771insSVD, and L858R mutated EGFR proteins; a DNA mutation encoding an E17K mutated AKT1 protein; a DNA mutation encoding an K57N mutated MEK1 protein; and a DNA mutation encoding an A775_G776insYVMA mutated HER2 protein. Any of the probes described herein (e.g., in section III) may be included in the kit, e.g., coupled to an encoded microcarrier. In some embodiments, the kit further comprises one or more microcarriers, probes, and/or primers corresponding to one or more RNA mutations, e.g., as described infra.

In some embodiments, the kit further comprises at least seven blocking nucleic acids. In some embodiments, said at least seven blocking nucleic acids hybridize with wild-type DNA loci corresponding with DNA mutations in the each of the KRAS, NRAS, PIK3CA, BRAF, EGFR, AKT1, MEK1, and HER2 genes. Exemplary descriptions of blocking nucleic acids are provided in section III.

In some embodiments, the kit further comprises one or more primer pairs, e.g., for amplifying the locus of a DNA mutation of interest. In some embodiments, the kit comprises a primer pair specific for the locus of one or more DNA mutations in each of the KRAS, NRAS, PIK3CA, BRAF, EGFR, AKT1, MEK1, and HER2 genes, e.g., at least seven primer pairs. Exemplary descriptions of primer pairs are provided in section III.

In some embodiments, a kit or article of manufacture of the present disclosure comprises: (a) a plurality of probes, wherein each probe of the plurality is coupled to a microcarrier that has a unique identifier corresponding to the probe coupled thereto, the plurality of probes comprising a first probe comprising a sequence selected from the group consisting of TAGTTGGAGCT (SEQ ID NO:38), TGTGGTAGTTG (SEQ ID NO:40), TGATGGCGTAG (SEQ ID NO:42), TGGAGCTGATGGC (SEQ ID NO:44), and GCGTAGGCAAG (SEQ ID NO:46); a second probe comprising a sequence selected from the group consisting of CTGTTGGCGTAGG (SEQ ID NO:48), GTAGTTGGAGCTG (SEQ ID NO:50), TGGAGCTGTTGGC (SEQ ID NO:52), TTGTGGTAGTTGG (SEQ ID NO:54), and GGCGTAGGCAAGA (SEQ ID NO:56); a third probe comprising a sequence selected from the group consisting of TAGTTGGAGCTT (SEQ ID NO:58), GCGTAGGCAAGA (SEQ ID NO:60), GGAGCTTGTGGC (SEQ ID NO:62), TTGTGGCGTAGG (SEQ ID NO:64), and TGTGGTAGTTGG (SEQ ID NO:66); a fourth probe comprising a sequence selected from the group consisting of GCTCAGTGATTTTAG (SEQ ID NO:87), TGCTCAGTGATTTT (SEQ ID NO:89), GCTCAGTGATTTTAG (SEQ ID NO:91), CCTGCTCAGTGATTTTA (SEQ ID NO:93), and CTCAGTGATTTTAGA (SEQ ID NO:95); a fifth probe comprising a sequence selected from the group consisting of TTCTCCTGCTTA (SEQ ID NO:97), CTCCTGCTTAGT (SEQ ID NO:99), TCTCCTGCTTAG (SEQ ID NO:101), TCCTGCTTAGTG (SEQ ID NO:103), and CTCCTGCTTAGTGA (SEQ ID NO:105); a sixth probe comprising a sequence selected from the group consisting of GATGCACGTCATG (SEQ ID NO:107), TGAATGATGCACG (SEQ ID NO:109), TGATGCACGTC (SEQ ID NO:111), AATGATGCACGTCA (SEQ ID NO:113), and AATGATGCACGTC (SEQ ID NO:115); a seventh probe comprising a sequence selected from the group consisting of TTTGGTCTAGCTACAGA (SEQ ID NO:79), CTACAGAGAAATCTCGA (SEQ ID NO:81), GTGATTTTGGTCTAGCT (SEQ ID NO:83), and TCTAGCTACAGAGAAAT (SEQ ID NO:85); an eighth probe comprising a sequence selected from the group consisting of TCAAAGTGCTGGCCTC (SEQ ID NO:117), AGATCAAAGTGCTGGCCTCCG (SEQ ID NO:119), AAAGTGCTGGCCT (SEQ ID NO:121), AGTGCTGGCCT (SEQ ID NO:123), and AAGTGCTGGCCTC (SEQ ID NO:125); a ninth probe comprising a sequence selected from the group consisting of AATCAAAACATCTCCGAAAG (SEQ ID NO:128), CAAAACATCTCCG (SEQ ID NO:130), AACATCTCCG (SEQ ID NO:132), and AAACATCTCCGAAAGCC (SEQ ID NO:134); a tenth probe comprising a sequence selected from the group consisting of AATCAAGACATCTCCGA (SEQ ID NO:136), GCAATCAAGACATCTCCGA (SEQ ID NO:138), AATCAAGACATCTC (SEQ ID NO:140), AATCAAGACATCTCCGAAAGC (SEQ ID NO:142), and CAAGACATCTCCGA (SEQ ID NO:144); an eleventh probe comprising a sequence selected from the group consisting of CCAGGAGGCTGCCG (SEQ ID NO:461), CAGGAGGCTGCCGA (SEQ ID NO:463), TCCAGGAGGCTGCC (SEQ ID NO:465), CCAGGAGGCTGCC (SEQ ID NO:467), and CAGGAGGCTGCC (SEQ ID NO:469); a thirteenth probe comprising a sequence selected from the group consisting of CCAGGAGGGAGCC (SEQ ID NO:471), CCAGGAGGGAGCCG (SEQ ID NO:473), TCCAGGAGGGAGCC (SEQ ID NO:475), CAGGAGGGAGCCG (SEQ ID NO:477), and CAGGAGGGAGCCGA (SEQ ID NO:479); a fourteenth probe comprising a sequence selected from the group consisting of ATGGCCATCTTGG (SEQ ID NO:421), GGCCATCTTGGA (SEQ ID NO:423), GATGGCCATCTTG (SEQ ID NO:425), TGATGGCCATCTTG (SEQ ID NO:427), and TGGCCATCTTGG (SEQ ID NO:429); a fifteenth probe comprising a sequence selected from the group consisting of GTGATGGCCGG (SEQ ID NO:431), TGATGGCCGGCG (SEQ ID NO:433), GTGATGGCCGGCGT (SEQ ID NO:435), GATGGCCGGCGT (SEQ ID NO:437), and GATGGCCCGCGTG (SEQ ID NO:439); a sixteenth probe comprising a sequence selected from the group consisting of AACCCCCATCACGT (SEQ ID NO:441), GACAACCCCCATCACG (SEQ ID NO:443), CGTGGACAACCCCCATCA (SEQ ID NO:445), CCCATCACGTGT (SEQ ID NO:447), and TGGACAACCCCCATCAC (SEQ ID NO:449); a seventeenth probe comprising a sequence selected from the group consisting of GCCAGCGTGGACGG (SEQ ID NO:451), CGTGGACGGTAACC (SEQ ID NO:453), GACGGTAACCCCC (SEQ ID NO:455), CCAGCGTGGACGGT (SEQ ID NO:457), and GCCAGCGTGGACGGTA (SEQ ID NO:459); an eighteenth probe comprising a sequence selected from the group consisting of ATTTTGGGCGGGCC (SEQ ID NO:151), TTGGGCGGGCCAAA (SEQ ID NO:153), GCGGGCCAAACT (SEQ ID NO:155), GGGCGGGCCAAACT (SEQ ID NO:157), and TGGGCGGGCCA (SEQ ID NO:159); a nineteenth probe comprising a sequence selected from the group consisting of TGTAGGGAAGTACA (SEQ ID NO:370), TCTGTAGGGAAGTAC (SEQ ID NO:372), GTCTGTAGGGAAGTACAT (SEQ ID NO:374), CCGCACGTCTGTAGGGA (SEQ ID NO:376), and ACGTCTGTAGGGAAGTA (SEQ ID NO:378); a twentieth probe comprising a sequence selected from the group consisting of TTACCCAGAATCAGAA (SEQ ID NO:387), CCAGAATCAGAAGGTG (SEQ ID NO:389), TTCTTACCCAGAATCA (SEQ ID NO:391), CCTTTCTTACCCAGAATC (SEQ ID NO:393), and CAGAATCAGAAGGTGG (SEQ ID NO:395); and a twenty-first probe comprising a sequence selected from the group consisting of ATACGTGATGTCTTAC (SEQ ID NO:404), ACGTGATGGCTTACGT (SEQ ID NO:406), AAGCATACGTGATGGCT (SEQ ID NO:408), GCATACGTGATGGCTT (SEQ ID NO:410), and GCATACGTGATGGCTTA (SEQ ID NO:412); (b) a plurality of primer pairs, the plurality of primer pairs comprising a first primer pair comprising the sequences GTACTGGTGGAGTATTTGATAGTG (SEQ ID NO:1) and CGTCAAGGCACTCTTGCCTAC (SEQ ID NO:2); a second primer pair comprising the sequences CAATTTCTACAAGAGATCCTCTCTCT (SEQ ID NO:5) and CTCCATTTTAGCACTTACCTGTGAC (SEQ ID NO:6); a third primer pair comprising the sequences ACCCTAGCCTTAGATAAAACTGAGC (SEQ ID NO:7) and TTTGTTGTCCAGCCACCATGA (SEQ ID NO:8); a fourth primer pair comprising the sequences ATAGCCTCAATTCTTACCATCCACAAAATG (SEQ ID NO:9) and CAGATATATTTCTTCATGAAGACCTCACAGTAA (SEQ ID NO: 10); a fifth primer pair comprising the sequences CTTGTGGAGCCTCTTACACCC (SEQ ID NO: 11) and TGCCGAACGCACCGGA (SEQ ID NO:12); a sixth primer pair comprising the sequences GCCAGTTAACGTCTTCCTTCTC (SEQ ID NO:13) and ATCGAGGATTTCCTTGTTGGCTT (SEQ ID NO:14); a seventh primer pair comprising the sequences CCTCCACCGTGCAGATCATC (SEQ ID NO:15) and TTCCCTGATTACCTTTGCGAT (SEQ ID NO:16); an eighth primer pair comprising the sequences CCACACTGACGTGCCTCT (SEQ ID NO: 511) and GCACACGTAGGGGTTGTCCAAGA (SEQ ID NO:512); a ninth primer pair comprising the sequences CCACACTGACGTGCCTCT (SEQ ID NO:513) and GTACACGCTGGCCACGCCG (SEQ ID NO:514); a tenth primer pair comprising the sequences CCACACTGACGTGCCTCT (SEQ ID NO:515) and CAGGCGGCACACGTGAT (SEQ ID NO:516); an eleventh primer pair comprising the sequences CCACACTGACGTGCCTCT (SEQ ID NO:517) and AGGCGGCACACGTGCGGGTTAC (SEQ ID NO:518); a twelfth primer pair comprising the sequences GGAGGACCGTCGCTTGG (SEQ ID NO:17) and TCTTTCTCTTCCGCACCCAG (SEQ ID NO:18); a thirteenth primer pair comprising the sequences GAGGGTCTGACGGGTAGAGTG (SEQ ID NO:380) and TGGCCGCCAGGTCTTGATGTA (SEQ ID NO:381), a fourteenth primer pair comprising the sequences CTTGATGAGCAGCAGCGAAA (SEQ ID NO:397) and CCTTCAGTTCTCCCACCTTCTG (SEQ ID NO:398); a fifteenth primer pair comprising the sequences ATGGCTGTGGTTTGTGATGGT (SEQ ID NO:414) and ACACCAGCCATCACGTAAGACA (SEQ ID NO:415); and (c) a plurality of blocking nucleic acids, the plurality of blocking nucleic acids comprising a first blocking nucleic acid comprising a sequence selected from the group consisting of TACGCCACCAGCT(invdT)ₙ, wherein n is 1, 2, or 3 (SEQ ID NO:281); TTGGAGCTGGTGGCGTA(invdT)ₙ, wherein n is 1, 2, or 3 (SEQ ID NO:282); GCTGGTGGCGTAGGCA(invdT), wherein n is 1, 2, or 3 (SEQ ID NO:283); GCTGGTGGCGTAGGC(invdT)ₙ, wherein n is 1, 2, or 3 (SEQ ID NO:284); and TTGGAGCTGGTGGCGT(invdT)ₙ, wherein n is 1, 2, or 3 (SEQ ID NO:285); a second blocking nucleic acid comprising a sequence selected from the group consisting of CTGAAATCACTGAGCAGG(invdT)ₙ, wherein n is 1, 2, or 3 (SEQ ID NO:291); TCTCTGAAATCACTGAGCAGG(invdT)ₙ, wherein n is 1, 2, or 3 (SEQ ID NO:292); TCTCTGAAATCACTGAGCAGG(invdT)ₙ, wherein n is 1, 2, or 3 (SEQ ID NO:293); TCTCTGAAATCACTGAGCAGG(invdT)ₙ, wherein n is 1, 2, or 3 (SEQ ID NO:294); and TCTCTGAATTCACTGAGCAGG(invdT)ₙ, wherein n is 1, 2, or 3 (SEQ ID NO:295); a third blocking nucleic acid comprising a sequence selected from the group consisting of CACCATGATGTGCAT(invdT)ₙ, wherein n is 1, 2, or 3 (SEQ ID NO:296); CCACCATGATGTGCAT(invdT)ₙ, wherein n is 1, 2, or 3 (SEQ ID NO:297); CACCATGATGTGCAT(invdT)ₙ, wherein n is 1, 2, or 3 (SEQ ID NO:298); CCACCATGATGTGCATCA(invdT)ₙ, wherein n is 1, 2, or 3 (SEQ ID NO:299); and CATGATGTGCA(invdT)ₙ, wherein n is 1, 2, or 3 (SEQ ID NO:300); a fourth blocking nucleic acid comprising a sequence selected from the group consisting of GAGATTTCACTGTAGC(invdT)ₙ, wherein n is 1, 2, or 3 (SEQ ID NO:301); GAGATTTCACTGTAGC(invdT)ₙ, wherein n is 1, 2, or 3 (SEQ ID NO:302); GAGATTTCACTGTAGC(invdT)ₙ, wherein n is 1, 2, or 3 (SEQ ID NO:303); GAGAITTCACTGTAGC(invdT)ₙ, wherein n is 1, 2, or 3 (SEQ ID NO:304); and GAGATTTCACTGTAGC(invdT)ₙ, wherein n is 1, 2, or 3 (SEQ ID NO: 305); a fifth blocking nucleic acid comprising a sequence selected from the group consisting of CGGAGCCCAGCACTTTGA (invdT)ₙ, wherein n is 1, 2, or 3 (SEQ ID NO:306); CGCACCGGAGCCCAGCACT (invdT)ₙ, wherein n is 1, 2, or 3 (SEQ ID NO:307); GAGCCCAGCAC (invdT)ₙ, wherein n is 1, 2, or 3 (SEQ ID NO:308); CGCACCGGAGCCCAGCAC (invdT)ₙ, wherein n is 1, 2, or 3 (SEQ ID NO:309); or CGCACCGGAGCCCAGCACTTA (invdT)ₙ, wherein n is 1, 2, or 3 (SEQ ID NO:310); a sixth blocking nucleic acid comprising a sequence selected from the group consisting of CGGAGATGTTGCTTCTCTTAATTCC(invdT)ₙ, wherein n is 1, 2, or 3 (SEQ ID NO:311); CGGAGATGTTGCTTCTCT(invdT)ₙ, wherein n is 1, 2, or 3 (SEQ ID NO: 312); GTTGCTTCTCTTAATTCC(invdT)ₙ, wherein n is 1, 2, or 3 (SEQ ID NO: 313); ATGTTGCTTCTCT(invdT)ₙ, wherein n is 1, 2, or 3 (SEQ ID NO:314); and TTGCTTCTCTTA(invdT)ₙ, wherein n is 1, 2, or 3 (SEQ ID NO:315); a seventh blocking nucleic acid comprising a sequence selected from the group consisting of CATCACGCAGCTCATG(invdT)ₙ, wherein n is 1, 2, or 3 (SEQ ID NO:316); TGCAGCTCATCACGCAGC(invdT)ₙ, wherein n is 1, 2, or 3 (SEQ ID NO:317); TCATCACGCAGCTCAT(invdT)ₙ, wherein n is 1, 2, or 3 (SEQ ID NO:318); TCATCACGCAGC(invdT)ₙ, wherein n is 1, 2, or 3 (SEQ ID NO:319), and CTCATCACGCAGC(invdT)ₙ, wherein n is 1, 2, or 3 (SEQ ID NO:320); an eighth blocking nucleic acid comprising a sequence selected from the group consisting of CCAGCAGTTTGGCCAGCCCT(invdT)ₙ, wherein n is 1, 2, or 3 (SEQ ID NO:321); CCAGCAGTTTGGCCAGCCCT(invdT)ₙ, wherein n is 1, 2, or 3 (SEQ ID NO:322); CCAGCAGTTTGGCCAGCCCT(invdT)ₙ, wherein n is 1, 2, or 3 (SEQ ID NO:323); AGCAGTTTGGCCAGCC(invdT)ₙ, wherein n is 1, 2, or 3 (SEQ ID NO:324); and CCAGCAGTTTGGCCAGCCCT(invdT)ₙ, wherein n is 1, 2, or 3 (SEQ ID NO:325); a ninth blocking nucleic acid comprising a sequence selected from the group consisting of TGTACTCCCCTACA (invdT)ₙ, wherein n is 1, 2, or 3 (SEQ ID NO:382); GATGTACTCCCCT (invdT)ₙ, wherein n is 1, 2, or 3 (SEQ ID NO:383); ATGTACTCCCCTAC (invdT)ₙ, wherein n is 1, 2, or 3 (SEQ ID NO:384); GTACTCCCCTACA (invdT)ₙ, wherein n is 1, 2, or 3 (SEQ ID NO:385); and GATGTACTCCCCTACA (invdT)ₙ, wherein n is 1, 2, or 3 (SEQ ID NO:386); and a tenth blocking nucleic acid comprising a sequence selected from the group consisting of TCTGCTTCTGGGTAAG (invdT)ₙ, wherein n is 1, 2, or 3 (SEQ ID NO:399); TTCTGCTTCTGGGTAAGA (invdT)ₙ, wherein n is 1, 2, or 3 (SEQ ID NO:400); CACCTTCTGCTTCTGGG (invdT)ₙ, wherein n is 1, 2, or 3 (SEQ ID NO:401); TCTGCTTCTGGGTA (invdT)ₙ, wherein n is 1, 2, or 3 (SEQ ID NO:402); and CACCTTCTGCTTCTGGGTAAGA (invdT)ₙ, wherein n is 1, 2, or 3 (SEQ ID NO:403); with italicized nucleic acids representing locked nucleic acids.

In some embodiments, a kit or article of manufacture of the present disclosure comprises at least five encoded microcarriers. In some embodiments, each of the seven encoded microcarriers comprises (i) a probe, specific for an RNA mutation in the ALK, ROS, RET, NTRK1, or cMET gene, coupled to the microcarrier; and (ii) an identifier corresponding to the probe. In some embodiments, the kit comprises at least one microcarrier comprising a probe specific for a DNA mutation in the ALK gene, at least one microcarrier comprising a probe specific for a DNA mutation in the ROS gene, at least one microcarrier comprising a probe specific for a DNA mutation in the RET gene, at least one microcarrier comprising a probe specific for a DNA mutation in the NTRK1 gene, and at least one microcarrier comprising a probe specific for a DNA mutation in the cMET gene That is to say, each of the ALK, ROS, RET, NTRK1, and cMET genes is represented in the kit by a microcarrier with a probe specific for a mutation in the gene. Exemplary ALK, ROS, RET, NTRK1, and cMET genes and mutations are described supra. In some embodiments, the ALK, ROS, RET, NTRK1, and cMET genes are human genes. In some embodiments, the kit comprises microcarriers with probes suitable for detecting each of the following mutations (e.g., with at least one microcarrier+probe species for each mutation in the kit): E13;A20, E20;A20, and E6;A20 ALK RNA mutations; C6;R32, C6;R34, S4;R32, and S4;R34 ROS RNA mutations; K15;R11, K15;R12, K16;R12, K22;R12, and K23;R12 RET RNA mutations; a C8;N12 NTRK1 RNA mutation; and an exon 14 skipping cMET RNA mutation. Any of the probes described herein (e.g., in section III) may be included in the kit, e.g., coupled to an encoded microcarrier. In some embodiments, the kit further comprises one or more microcarriers, probes, and/or primers corresponding to one or more DNA mutations, e.g., as described supra.

In some embodiments, the kit further comprises at least five blocking nucleic acids. In some embodiments, said at least seven blocking nucleic acids hybridize with wild-type DNA loci (e.g., amplified from RNA via PCR amplification of cDNA) corresponding with RNA mutations in the each of the ALK, ROS, RET, NTRK1, and cMET genes. Exemplary descriptions of blocking nucleic acids are provided in section III.

In some embodiments, the kit further comprises one or more primer pairs, e.g., for amplifying the locus of a RNA mutation of interest. In some embodiments, the kit comprises a primer pair specific for the locus of one or more RNA mutations in each of the ALK, ROS, RET, NTRK1, and cMET genes, e.g., at least five primer pairs (e.g., a first primer for generation of cDNA and a second primer for PCR amplification of DNA from the cDNA in combination with the first primer). Exemplary descriptions of primer pairs are provided in section III.

In some embodiments, a kit or article of manufacture of the present disclosure comprises: (a) a plurality of probes, wherein each probe of the plurality is coupled to a microcarrier that has a unique identifier corresponding to the probe coupled thereto, the plurality of probes comprising a first probe comprising a sequence selected from the group consisting of AAAGGACCTAAAGTGT (SEQ ID NO:161). CCTAAAGTGTACCGC (SEQ ID NO:163), GGGAAAGGACCTAAAG (SEQ ID NO:165), AGTGTACCGCCGGAA (SEQ ID NO:167), and TACCGCCGGAAGCACC (SEQ ID NO:169); a second probe comprising a sequence selected from the group consisting of GACTATGAAATATTGTAC (SEQ ID NO:171), GAAATATTGTACTTGTAC (SEQ ID NO:173), TATTGTACTTGTACCGCC (SEQ ID NO:175), TGTACCGCCGGAAGCAC (SEQ ID NO:177), and CCGCCGGAAGCACCAGGA (SEQ ID NO:179); a third probe comprising a sequence selected from the group consisting of TGTCATCATCAACCAA (SEQ ID NO:181), ATGTCATCATCAACC (SEQ ID NO:183), GTGTACCGCCGGAAGC (SEQ ID NO:185), TCAACCAAGTGTACCG (SEQ ID NO:187), and TACCGCCGGAAGCACCA (SEQ ID NO:189); a fourth probe comprising a sequence selected from the group consisting of CGAAAAAAACAGCCAA (SEQ ID NO:191), TCGCGAAAAAAACAGC (SEQ ID NO:193), GTGTACCGCCGGAAGC (SEQ ID NO 195), TACCGCCGGAAGCACC (SEQ ID NO:197), and ACAGCCAAGTGTACCG (SEQ ID NO:199); a fifth probe comprising a sequence selected from the group consisting of ACTGACGCTCCACCGAAA (SEQ ID NO:201), CCACTGACGCTCCACCGA (SEQ ID NO:203), GCTGGAGTCCCAAATAAAC (SEQ ID NO:205), GGAGTCCCAAATAAACCAG (SEQ ID NO:207), and CACCGAAAGCTGGAGTCCC (SEQ ID NO:209); a sixth probe comprising a sequence selected from the group consisting of CCGAAAGATGATTTT (SEQ ID NO:211), GACGCTCCACCGAAA (SEQ ID NO:213), ACTGACGCTCCACCGA (SEQ ID NO: 215), GATGATTTTTGGATA (SEQ ID NO:217), and TGATTTTTGGATACCA (SEQ ID NO:219); a seventh probe comprising a sequence selected from the group consisting of AGCGCCTTCCAGCTGGTTGGA (SEQ ID NO:221), CTGGTTGGAGCTGGAGTCCC (SEQ ID NO:223), AGTAGCGCCTTCCAGCTGGTTG (SEQ ID NO:225), GCTGGAGTCCCAAATAAACCA (SEQ ID NO:227), and GGAGTCCCAAATAAACCAGG (SEQ ID NO:229); an eighth probe comprising a sequence selected from the group consisting of GCGCCTTCCAGCTGGTTG (SEQ ID NO:231), GTAGCGCCTTCCAGCTGGT (SEQ ID NO:233), TGGTTGGAGATGATTTTT (SEQ ID NO:235), GATGATTTTTGGATACCAG (SEQ ID NO:237), and TGATTTTTGGATACCA (SEQ ID NO:239); a ninth probe comprising a sequence selected from the group consisting of GTGGGAAATAATGATGTAAA (SEQ ID NO:241), CTGTGGGAAATAATGATGTA (SEQ ID NO:243), GATCCACTGTGCGACGAGCT (SEQ ID NO:245), TGATGTAAAGATCCACTGTG (SEQ ID NO:247), and TCCACTGTGCGACGAGCTGT (SEQ ID NO:249); a tenth probe comprising a sequence selected from the group consisting of TGGGAAATAATGATGTAAA (SEQ ID NO:251), CTGTGGGAAATAATGATGTA (SEQ ID NO:253), GGAGGATCCAAAGTGGGAAT (SEQ ID NO:255), GGATCCAAAGTGGGAATT (SEQ ID NO:257), and ATGATGTAAAGGAGGATCC (SEQ ID NO:259); an eleventh probe comprising a sequence selected from the group consisting of CTTCGTATCTCTCAAGAGGAT (SEQ ID NO:481), GTATCTCTCAAGAGGATCCAA (SEQ ID NO:483), TTCGTATCTCTCAAGAG (SEQ ID NO:485), TCAAGAGGATCCAAA (SEQ ID NO:487), and TCTCTCAAGAGG (SEQ ID NO:489); a twelfth probe comprising a sequence selected from the group consisting of GTTAAAAAGGAGGATCCAA (SEQ ID NO:491), ACAAGAGTTAAAAAGGAGGA (SEQ ID NO:493), AAGAGTTAAAAAGGAGGATC (SEQ ID NO:495), AAAAGGAGGATCCAAAG (SEQ ID NO:497), and AAGGAGGATCCAAAGTG (SEQ ID NO:499); a thirteenth probe comprising a sequence selected from the group consisting of AAACAGGAGGATCCAAA (SEQ ID NO:501), AAGTGCACAAACAGGAGG (SEQ ID NO:503), GTGCACAAACAGGAGGATC (SEQ ID NO:505), CACAAACAGGAGGAT (SEQ ID NO:507), and AACAGGAGGATCCAAA (SEQ ID NO:509); a fourteenth probe comprising a sequence selected from the group consisting of CAGGATCTGGGCCCAGACA (SEQ ID NO:261), GATCTGGGCCCAGACACTA (SEQ ID NO:263), CCAGACACTAACAGCACAT (SEQ ID NO:265), GGGCCCAGACACTAACAGC (SEQ ID NO:267), and CTAACAGCACATCTGGAGA (SEQ ID NO:269); and a fifteenth probe comprising a sequence selected from the group consisting of AGAAAGCAAATTAAAGAT (SEQ ID NO:271), AGCAAATTAAAGATCAG (SEQ ID NO:273), AAATTAAAGATCAGTTTC (SEQ ID NO:275), AGATCAGTTTCCTAATTC (SEQ ID NO:277), and AAGATCAGTTTCCTAATT (SEQ ID NO:279); and (b) a plurality of primers, the plurality of primers comprising each of the following sequences (with at least one individual, respective primer per sequence) AGTTGGGGTTGTAGTCGGTCAT (SEQ ID NO:363), AGCCTCCCTGGATCTCC (SEQ ID NO:364), TATGGAGCAAAACTACTGTAGAGCC (SEQ ID NO:357), CCAGCTACATCACACACCTTGACT (SEQ ID NO:358), TAATACCAAAAGTTACCAAAACTGCA (SEQ ID NO:359), GGAGTGCCATCGCTGTTTGAAATGAGCAGGCACT (SEQ ID NO:19), TACAGCCCTGGATATTCTTAGTAGCGC (SEQ ID NO:20), GTGATCGCACAGTAGGACAGCGGCTGCGATC (SEQ ID NO:26), CTCTAGGAGATATCATTCCAAATTCGCCTTCTCCTAG (SEQ ID NO:27), TTTCTGGTGCTATGAGGAAATGACCAACCACCAGA (SEQ ID NO:23), AAGGAGTTAGCAGCATGTCAGC (SEQ ID NO:519), AACTTCAGACTTTACACAACCTGC (SEQ ID NO:520), ATTGATTCTGATGACACCGGA (SEQ ID NO:521), GGACGAAAATCCAGACCCCAAAAGGTGTTTCGT (SEQ ID NO:32), CATTGGGTGACATCAGGGAGCTGCCACCCAA (SEQ ID NO:33), AGAAGACGTGACAGGAACTGGAGGACCCGTCTT (SEQ ID NO:30), GACAGTATTTTGCAGTAATGGACTGGATATATCAGA (SEQ ID NO:29), and GAATTTCACAGGATTGATTGCTGGTGTTGTCTC (SEQ ID NO:28).

It is to be understood that a kit of the present disclosure can comprise various microcarrier/probes, primers, and/or blocking nucleic acids suitable for detecting one or more DNA mutations and/or one or more RNA mutations of the present disclosure. Non-limiting examples of such kits are described below, but it is contemplated that any of the reagents for detecting any of the DNA/RNA mutations of the present disclosure can be combined in any number or combination.

In some embodiments, a kit or article of manufacture of the present disclosure comprises: (a) a plurality of probes, wherein each probe of the plurality is coupled to a microcarrier that has a unique identifier corresponding to the probe coupled thereto, the plurality of probes comprising a first probe comprising the sequence TTTTTTTTTTTTAATAGTTGGAGCT (SEQ ID NO:39); a second probe comprising the sequence TTTTTTTTTTTAGGCGTAGGCAAGA (SEQ ID NO:57); a third probe comprising the sequence TTTTTTTTTTTAAGGAGCTTGTGGC (SEQ ID NO: 63); a fourth probe comprising the sequence TTTTTTTCCTGCTCAGTGATTTTA (SEQ ID NO:94); a fifth probe comprising the sequence TTTTTTTTTTTATCTCCTGCTTAG (SEQ ID NO:102); a sixth probe comprising the sequence TTTTTTTTTTTTAATGATGCACGTCA (SEQ ID NO:114); a seventh probe comprising the sequence TTTTTTAATTCTACAGAGAAATCTCGA (SEQ ID NO:82); an eighth probe comprising the sequence TTTTTTTTTTTTTAGTGCTGGCCT (SEQ ID NO:124); a ninth probe comprising the sequence TTTTTTTTTTTACAAAACATCTCCG (SEQ ID NO:131); a tenth probe comprising the sequence TTTTTTTTAATCAAGACATCTC (SEQ ID NO: 141); an eleventh probe comprising the sequence TTTTTTTTCATGAGATGCATGATGA (SEQ ID NO:356); a twelfth probe comprising the sequence TTTTTTTTTTTACAGGAGGCTGCCGA (SEQ ID NO:464); a thirteenth probe comprising the sequence TTTTTTTTTTTACAGGAGGGAGCCG (SEQ ID NO:478); a fourteenth probe comprising the sequence TTTTTTTAGATGGCCATCTTG (SEQ ID NO:426); a fifteenth probe comprising the sequence TTTTTTTTTTTGTGATGGCCGG (SEQ ID NO:432); a sixteenth probe comprising the sequence TTTTTTTTGGACAACCCCCATCAC (SEQ ID NO:450); a seventeenth probe comprising the sequence TTTTTTTGCCAGCGTGGACGGTA (SEQ ID NO:460); an eighteenth probe comprising the sequence TTTTTTTTAAATGGGCGGGCCA (SEQ ID NO:160); a nineteenth probe comprising the sequence TTTTTTTTTTTTTTGTAGGGAAGTACA (SEQ ID NO:371); a twentieth probe comprising the sequence TTTTTAAATCAGAATCAGAAGGTGG (SEQ ID NO:396); a twentieth probe comprising the sequence TTTTTAAATCAGAATCAGAAGGTGG (SEQ ID NO:396); a twenty-first probe comprising the sequence TTTTTTTTTTTACGTGATGGCTTACGT (SEQ ID NO:407); a twenty-second probe comprising the sequence TTTTTTTTTTAGTGTACCGCCGGAA (SEQ ID NO:168); a twenty-third probe comprising the sequence TTTTTTTTTTTTGACTATGAAATATTGTAC (SEQ ID NO:172); a twenty-fourth probe comprising the sequence TTTTTTTTTTTTTTTACCGCCGGAAGCACCA (SEQ ID NO:190); a twenty-fifth probe comprising the sequence TTTTTTTTTTTTTGTGTACCGCCGGAAGC (SEQ ID NO:196); a twenty-sixth probe comprising the sequence TTTTTTTTTTTGCTGGAGTCCCAAATAAAC (SEQ ID NO:206); a twenty-seventh probe comprising the sequence TTTTTTTTTTTTGACGCTCCACCGAAA (SEQ ID NO:214); a twenty-eighth probe comprising the sequence TTTTTTTTTTTTGGAGTCCCAAATAAACCAGG (SEQ ID NO:230); a twenty-ninth probe comprising the sequence TTTTTTTTTTGATGATTTTTGGATACCAG (SEQ ID NO:238); a thirtieth probe comprising the sequence TTTTTTTTTTGTGGGAAATAATGATGTAAA (SEQ ID NO:242); a thirty-first probe comprising the sequence TTTTTTTTTCTGTGGGAAATAATGATGTA (SEQ ID NO:254); a thirty-second probe comprising the sequence TTTTTTTTTTCTCTCAAGAGG (SEQ ID NO:490); a thirty-third probe comprising the sequence TTTTTTTTAAGGAGGATCCAAAGTG (SEQ ID NO:500); a thirty-fourth probe comprising the sequence TTTTTTTTAAACAGGAGGATCCAAA (SEQ ID NO:502); a thirty-fifth probe comprising the sequence TTTTTTTTTTACCAGACACTAACAGCACAT (SEQ ID NO:266); a thirty-sixth probe comprising the sequence TTTTTTTTTTAGAAAGCAAATTAAAGAT (SEQ ID NO:272); (b) a plurality of primer pairs, the plurality of primer pairs comprising a first primer pair comprising the sequences GTACTGGTGGAGTATTTGATAGTG (SEQ ID NO:1) and CGTCAAGGCACTCTTGCCTAC (SEQ ID NO:2); a second primer pair comprising the sequences CAATTTCTACAAGAGATCCTCTCTCT (SEQ ID NO:5) and CTCCATTTTAGCACTTACCTGTGAC (SEQ ID NO:6); a third primer pair comprising the sequences ACCCTAGCCTTAGATAAAACTGAGC (SEQ ID NO:7) and TTTGTTGTCCAGCCACCATGA (SEQ ID NO:8); a fourth primer pair comprising the sequences ATAGCCTCAATTCTTACCATCCACAAAATG (SEQ ID NO:9) and CAGATATATTTCTTCATGAAGACCTCACAGTAA (SEQ ID NO:10); a fifth primer pair comprising the sequences CTTGTGGAGCCTCTTACACCC (SEQ ID NO: 11) and TGCCGAACGCACCGGA (SEQ ID NO:12); a sixth primer pair comprising the sequences GCCAGTTAACGTCTTCCTTCTC (SEQ ID NO: 13) and ATCGAGGATTTCCTTGTTGGCTT (SEQ ID NO:14); a seventh primer pair comprising the sequences CCTCCACCGTGCAGATCATC (SEQ ID NO:15) and TTCCCTGATTACCTTTGCGAT (SEQ ID NO:16); an eighth primer pair comprising the sequences CCACACTGACGTGCCTCT (SEQ ID NO:511) and GCACACGTAGGGGTTGTCCAAGA (SEQ ID NO:512); a ninth primer pair comprising the sequences CCACACTGACGTGCCTCT (SEQ ID NO: 513) and GTACACGCTGGCCACGCCG (SEQ ID NO:514); a tenth primer pair comprising the sequences CCACACTGACGTGCCTCT (SEQ ID NO:515) and CAGGCGGCACACGTGAT (SEQ ID NO:516); an eleventh primer pair comprising the sequences CCACACTGACGTGCCTCT (SEQ ID NO:517) and AGGCGGCACACGTGCGGGTTAC (SEQ ID NO:518); a twelfth primer pair comprising the sequences GGAGGACCGTCGCTTGG (SEQ ID NO:17) and TCTTTCTCTTCCGCACCCAG (SEQ ID NO:18); a thirteenth primer pair comprising the sequences GAGGGTCTGACGGGTAGAGTG (SEQ ID NO:380) and TGGCCGCCAGGTCTTGATGTA (SEQ ID NO:381), a fourteenth primer pair comprising the sequences CTTGATGAGCAGCAGCGAAA (SEQ ID NO:397) and CCTTCAGTTCTCCCACCTTCTG (SEQ ID NO:398); a fifteenth primer pair comprising the sequences ATGGCTGTGGTTTGTGATGGT (SEQ ID NO:414) and ACACCAGCCATCACGTAAGACA (SEQ ID NO:415); a sixteenth primer pair comprising the sequences AGTTGGGGTTGTAGTCGGTCAT (SEQ ID NO:363) and TATGGAGCAAAACTACTGTAGAGCC (SEQ ID NO: 357); a seventeenth primer pair comprising the sequences AGTTGGGGTTGTAGTCGGTCAT (SEQ ID NO:363) and CCAGCTACATCACACACCTTGACT (SEQ ID NO:358); an eighteenth primer pair comprising the sequences AGTTGGGGTTGTAGTCGGTCAT (SEQ ID NO:363) and TAATACCAAAAGTTACCAAAACTGCA (SEQ ID NO:359); a nineteenth primer pair comprising the sequences GGAGTGCCATCGCTGTTTGAAATGAGCAGGCACT (SEQ ID NO:19); a twentieth primer pair comprising the sequences GGAGTGCCATCGCTGTTTGAAATGAGCAGGCACT (SEQ ID NO:19); a twenty-first primer pair comprising the sequences TACAGCCCTGGATATTCTTAGTAGCGC (SEQ ID NO:20); a twenty-second primer pair comprising the sequences TACAGCCCTGGATATTCTTAGTAGCGC (SEQ ID NO:20); a twenty-third primer pair comprising the sequences GTGATCGCACAGTAGGACAGCGGCTGCGATC (SEQ ID NO:26) and TTTCTGGTGCTATGAGGAAATGACCAACCACCAGA (SEQ ID NO: 23); a twenty-fourth primer pair comprising the sequences CTCTAGGAGATATCATTCCAAATTCGCCTTCTCCTAG (SEQ ID NO:27) and TTTCTGGTGCTATGAGGAAATGACCAACCACCAGA (SEQ ID NO:23); a twenty-fifth primer pair comprising the sequences CTCTAGGAGATATCATTCCAAATTCGCCTTCTCCTAG (SEQ ID NO:27) and AAGGAGTTAGCAGCATGTCAGC (SEQ ID NO:519); a twenty-sixth primer pair comprising the sequences CTCTAGGAGATATCATTCCAAATTCGCCTTCTCCTAG (SEQ ID NO:27) and AACTTCAGACTTTACACAACCTGC (SEQ ID NO:520); a twenty-seventh primer pair comprising the sequences CTCTAGGAGATATCATTCCAAATTCGCCTTCTCCTAG (SEQ ID NO:27) and ATTGATTCTGATGACACCGGA (SEQ ID NO:521); a twenty-eighth primer pair comprising the sequences GGACGAAAATCCAGACCCCAAAAGGTGTTTCGT (SEQ ID NO:32) and AGAAGACGTGACAGGAACTGGAGGACCCGTCTT (SEQ ID NO:30); a twenty-ninth primer pair comprising the sequences GACAGTATTTTGCAGTAATGGACTGGATATATCAGA (SEQ ID NO:29) and GAATTTCACAGGATTGATTGCTGGTGTTGTCTC (SEQ ID NO:28); and (c) a plurality of blocking nucleic acids, the plurality of blocking nucleic acids comprising a first blocking nucleic acid comprising the sequence TTGGAGCTGGTGGCGT(invdT)ₙ, wherein n is 1, 2, or 3 (SEQ ID NO:285); a second blocking nucleic acid comprising the sequence CTGAAATCACTGAGCAGG(invdT)ₙ, wherein n is 1, 2, or 3 (SEQ ID NO:291); a third blocking nucleic acid comprising the sequence CCACCATGATGTGCATCA(invdT)ₙ, wherein n is 1, 2, or 3 (SEQ ID NO:299); a fourth blocking nucleic acid comprising the sequence GAGATTTCACTGTAGC(invdT)ₙ, wherein n is 1, 2, or 3 (SEQ ID NO:301); a fifth blocking nucleic acid comprising the sequence CGCACCGGAGCCCAGCACTTA (invdT)ₙ, wherein n is 1, 2, or 3 (SEQ ID NO:310); a sixth blocking nucleic acid comprising the sequence CGGAGATGTTGCTTCTCT(invdT)ₙ, wherein n is 1, 2, or 3 (SEQ ID NO:312); a seventh blocking nucleic acid comprising the sequence TGCAGCTCATCACGCAGC(invdT)ₙ, wherein n is 1, 2, or 3 (SEQ ID NO:317); an eighth blocking nucleic acid comprising the sequence CCAGCAGTTTGGCCAGCCCT(invdT)ₙ, wherein n is 1, 2, or 3 (SEQ ID NO:322); a ninth blocking nucleic acid comprising the sequence GATGTACTCCCCT (invdT)ₙ, wherein n is 1, 2, or 3 (SEQ ID NO:383); and a tenth blocking nucleic acid comprising the sequence CACCTTCTGCTTCTGGGTAAGA (invdT)ₙ, wherein n is 1, 2, or 3 (SEQ ID NO:403).

In some embodiments, a kit or article of manufacture of the present disclosure comprises: (a) a plurality of probes, wherein each probe of the plurality is coupled to a microcarrier that has a unique identifier corresponding to the probe coupled thereto, the plurality of probes comprising a first probe comprising the sequence TTTTTTTTTTTTAATGATGGCGTAG (SEQ ID NO:43); a second probe comprising the sequence TTTTTTTTTTTAGTAGTTGGAGCTG (SEQ ID NO:51); a third probe comprising the sequence TTTTTTTTTTTAATTGTGGCGTAGG (SEQ ID NO:65); a fourth probe comprising the sequence TTTTTTTTTAGCTCAGTGATTTTAG (SEQ ID NO:88); a fifth probe comprising the sequence TTTTTTTTTTTTTTTCCTGCTTAGTG (SEQ ID NO:104); a sixth probe comprising the sequence TTTTTTTTTTTTTGATGCACGTC (SEQ ID NO:112); a seventh probe comprising the sequence TTTTTTAATTGAGAAATCTCGATGGAG (SEQ ID NO:78); an eighth probe comprising the sequence TTTTTTAGATCAAAGTGCTGGCCTCCG (SEQ ID NO:120); a ninth probe comprising the sequence TTTTTTTTTTTTTTAAACATCTCCGAAAGCC (SEQ ID NO:135); a tenth probe comprising the sequence TTTTTTTTTTTCAAGACATCTCCGA (SEQ ID NO:145); an eleventh probe comprising the sequence TTTTTTTTTTTGAGATGCATGATGA (SEQ ID NO:352); a twelfth probe comprising the sequence TTTTTTTTTTTACAGGAGGCTGCC (SEQ ID NO:470); a thirteenth probe comprising the sequence TTTTTTTTTTTACCAGGAGGGAGCCG (SEQ ID NO:474); a fourteenth probe comprising the sequence TTTTTTTTTATGGCCATCTTGG (SEQ ID NO:422); a fifteenth probe comprising the sequence TTTTTTTTTTTTTGATGGCCCGCGTG (SEQ ID NO:440); a sixteenth probe comprising the sequence TTTTTTTTTTTTCCCATCACGTGT (SEQ ID NO:448); a seventeenth probe comprising the sequence TTTTTTTTTTTGACGGTAACCCCC (SEQ ID NO:456); an eighteenth probe comprising the sequence TTTTTTTATTTTGGGCGGGCC (SEQ ID NO:152); a nineteenth probe comprising the sequence TTTTTTTTTTTTCTGTAGGGAAGTAC (SEQ ID NO:373); a twentieth probe comprising the sequence TTTTTAAATTTCTTACCCAGAATCA (SEQ ID NO:392); a twenty-first probe comprising the sequence TTTTTAAGCATACGTGATGGCT (SEQ ID NO:409); a twenty-second probe comprising the sequence TTTTTTTTTTGGGAAAGGACCTAAAG (SEQ ID NO:166); a twenty-third probe comprising the sequence ITTTTTTTTTTTCCGCCGGAAGCACCAGGA (SEQ ID NO:180); a twenty-fourth probe comprising the sequence TTTTTTTTTTTTTTGTGTACCGCCGGAAGC (SEQ ID NO:186); a twenty-fifth probe comprising the sequence TTTTTTTTTTTTTCGAAAAAAACAGCCAA (SEQ ID NO:192); a twenty-sixth probe comprising the sequence TTTTTTTTTTTACTGACGCTCCACCGAAA (SEQ ID NO:202); a twenty-seventh probe comprising the sequence TTTTTTTTTTTTGACGCTCCACCGAAA (SEQ ID NO:214); a twenty-eighth probe comprising the sequence TTTTTTTTTTTTAGCGCCTTCCAGCTGGTTGGA (SEQ ID NO:222); a twenty-ninth probe comprising the sequence TTTTTTTTTTTGATTTTTGGATACCA (SEQ ID NO:240); a thirtieth probe comprising the sequence TTTTTTTTTTTCCACTGTGCGACGAGCTGT (SEQ ID NO:250); a thirty-first probe comprising the sequence TTTTTTTTTGGATCCAAAGTGGGAATT (SEQ ID NO:258); a thirty-second probe comprising the sequence TTTTTTTTTTCAAGAGGATCCAAA (SEQ ID NO:488); a thirty-third probe comprising the sequence TTTTTTTTACAAGAGTTAAAAAGGAGGA (SEQ ID NO:494); a thirty-fourth probe comprising the sequence TTTTTATTAAGTGCACAAACAGGAGG (SEQ ID NO:504); a thirty-fifth probe comprising the sequence TTTTTTTTTTACTAACAGCACATCTGGAGA (SEQ ID NO:270); a thirty-sixth probe comprising the sequence TTTTTTTTTTAGATCAGTTTCCTAATTC (SEQ ID NO:278); (b) a plurality of primer pairs, the plurality of primer pairs comprising a first primer pair comprising the sequences GTACTGGTGGAGTATTTGATAGTG (SEQ ID NO:1) and CGTCAAGGCACTCTTGCCTAC (SEQ ID NO:2); a second primer pair comprising the sequences CAATTTCTACAAGAGATCCTCTCTCT (SEQ ID NO:5) and CTCCATTTTAGCACTTACCTGTGAC (SEQ ID NO:6); a third primer pair comprising the sequences ACCCTAGCCTTAGATAAAACTGAGC (SEQ ID NO:7) and TTTGTTGTCCAGCCACCATGA (SEQ ID NO:8); a fourth primer pair comprising the sequences ATAGCCTCAATTCTTACCATCCACAAAATG (SEQ ID NO:9) and CAGATATATTTCTTCATGAAGACCTCACAGTAA (SEQ ID NO:10); a fifth primer pair comprising the sequences CTTGTGGAGCCTCTTACACCC (SEQ ID NO: 11) and TGCCGAACGCACCGGA (SEQ ID NO:12); a sixth primer pair comprising the sequences GCCAGTTAACGTCTTCCTTCTC (SEQ ID NO: 13) and ATCGAGGATTTCCTTGTTGGCTT (SEQ ID NO:14); a seventh primer pair comprising the sequences CCTCCACCGTGCAGATCATC (SEQ ID NO:15) and TTCCCTGATTACCTTTGCGAT (SEQ ID NO:16); an eighth primer pair comprising the sequences CCACACTGACGTGCCTCT (SEQ ID NO:511) and GCACACGTAGGGGTTGTCCAAGA (SEQ ID NO:512); a ninth primer pair comprising the sequences CCACACTGACGTGCCTCT (SEQ ID NO:513) and GTACACGCTGGCCACGCCG (SEQ ID NO:514); a tenth primer pair comprising the sequences CCACACTGACGTGCCTCT (SEQ ID NO:515) and CAGGCGGCACACGTGAT (SEQ ID NO:516); an eleventh primer pair comprising the sequences CCACACTGACGTGCCTCT (SEQ ID NO:517) and AGGCGGCACACGTGCGGGTTAC (SEQ ID NO:518); a twelfth primer pair comprising the sequences GGAGGACCGTCGCTTGG (SEQ ID NO:17) and TCTTTCTCTTCCGCACCCAG (SEQ ID NO:18); a thirteenth primer pair comprising the sequences GAGGGTCTGACGGGTAGAGTG (SEQ ID NO: 380) and TGGCCGCCAGGTCTTGATGTA (SEQ ID NO:381); a fourteenth primer pair comprising the sequences CTTGATGAGCAGCAGCGAAA (SEQ ID NO:397) and CCTTCAGTTCTCCCACCTTCTG (SEQ ID NO:398); a fifteenth primer pair comprising the sequences ATGGCTGTGGTTTGTGATGGT (SEQ ID NO:414) and ACACCAGCCATCACGTAAGACA (SEQ ID NO:415); a sixteenth primer pair comprising the sequences AGTTGGGGTTGTAGTCGGTCAT (SEQ ID NO:363) and TATGGAGCAAAACTACTGTAGAGCC (SEQ ID NO:357); a seventeenth primer pair comprising the sequences AGTTGGGGTTGTAGTCGGTCAT (SEQ ID NO:363) and CCAGCTACATCACACACCTTGACT (SEQ ID NO:358); an eighteenth primer pair comprising the sequences AGTTGGGGTTGTAGTCGGTCAT (SEQ ID NO:363) and TAATACCAAAAGTTACCAAAACTGCA (SEQ ID NO:359); a nineteenth primer pair comprising the sequences GGAGTGCCATCGCTGTTTGAAATGAGCAGGCACT (SEQ ID NO:19); a twentieth primer pair comprising the sequences GGAGTGCCATCGCTGTTTGAAATGAGCAGGCACT (SEQ ID NO:19); a twenty-first primer pair comprising the sequences TACAGCCCTGGATATTCTTAGTAGCGC (SEQ ID NO:20); a twenty-second primer pair comprising the sequences TACAGCCCTGGATATTCTTAGTAGCGC (SEQ ID NO:20); a twenty-third primer pair comprising the sequences GTGATCGCACAGTAGGACAGCGGCTGCGATC (SEQ ID NO:26) and TTTCTGGTGCTATGAGGAAATGACCAACCACCAGA (SEQ ID NO:23); a twenty-fourth primer pair comprising the sequences CTCTAGGAGATATCATTCCAAATTCGCCTTCTCCTAG (SEQ ID NO:27) and TTTCTGGTGCTATGAGGAAATGACCAACCACCAGA (SEQ ID NO:23); a twenty-fifth primer pair comprising the sequences CTCTAGGAGATATCATTCCAAATTCGCCTTCTCCTAG (SEQ ID NO:27) and AAGGAGTTAGCAGCATGTCAGC (SEQ ID NO:519); a twenty-sixth primer pair comprising the sequences CTCTAGGAGATATCATTCCAAATTCGCCTTCTCCTAG (SEQ ID NO:27) and AACTTCAGACTTTACACAACCTGC (SEQ ID NO:520); a twenty-seventh primer pair comprising the sequences CTCTAGGAGATATCATTCCAAATTCGCCTTCTCCTAG (SEQ ID NO:27) and ATTGATTCTGATGACACCGGA (SEQ ID NO:521); a twenty-eighth primer pair comprising the sequences GGACGAAAATCCAGACCCCAAAAGGTGTTTCGT (SEQ ID NO:32) and AGAAGACGTGACAGGAACTGGAGGACCCGTCTT (SEQ ID NO:30); a twenty-ninth primer pair comprising the sequences GACAGTATTTTGCAGTAATGGACTGGATATATCAGA (SEQ ID NO:29) and GAATTTCACAGGATTGATTGCTGGTGTTGTCTC (SEQ ID NO:28); and (c) a plurality of blocking nucleic acids, the plurality of blocking nucleic acids comprising a first blocking nucleic acid comprising the sequence TTGGAGCTGGTGGCGTA(invdT)ₙ, wherein n is 1, 2, or 3 (SEQ ID NO:282); a second blocking nucleic acid comprising the sequence TCTCTGAAATCACTGAGCAGG(invdT)ₙ, wherein n is 1, 2, or 3 (SEQ ID NO:294); a third blocking nucleic acid comprising the sequence CCACCATGATGTGCAT(invdT)ₙ, wherein n is 1, 2, or 3 (SEQ ID NO:297); a fourth blocking nucleic acid comprising the sequence GAGATTTCACTGTAGC(invdT)ₙ, wherein n is 1, 2, or 3 (SEQ ID NO:305); a fifth blocking nucleic acid comprising the sequence CGCACCGGAGCCCAGCAC (invdT)ₙ, wherein n is 1, 2, or 3 (SEQ ID NO:309); a sixth blocking nucleic acid comprising the sequence GTTGCTTCTCTTAATTCC(invdT)ₙ, wherein n is 1, 2, or 3 (SEQ ID NO:313); a seventh blocking nucleic acid comprising the sequence CTCATCACGCAGC(invdT)ₙ, wherein n is 1, 2, or 3 (SEQ ID NO:320); an eighth blocking nucleic acid comprising the sequence CCAGCAGTTTGGCCAGCCCT(invdT)ₙ, wherein n is 1, 2, or 3 (SEQ ID NO:325); a ninth blocking nucleic acid comprising the sequence; GATGTACTCCCCTACA (invdT)ₙ, wherein n is 1, 2, or 3 (SEQ ID NO:386); and a tenth blocking nucleic acid comprising the sequence CACCTTCTGCTTCTGGG (invdT)ₙ, wherein n is 1, 2, or 3 (SEQ ID NO:401).

In some embodiments, a kit or article of manufacture of the present disclosure comprises: (a) a plurality of probes, wherein each probe of the plurality is coupled to a microcarrier that has a unique identifier corresponding to the probe coupled thereto, the plurality of probes comprising a first probe comprising the sequence TTTTTTTTTTTATGGAGCTGATGGC (SEQ ID NO:45); a second probe comprising the sequence TTTTTTTTTTTATGGAGCTGTTGGC (SEQ ID NO:53); a third probe comprising the sequence TTTTTTTTTTTAAGGAGCTTGTGGC (SEQ ID NO: 63); a fourth probe comprising the sequence TTTTTTTTTTTCTCAGTGATTTTAGA (SEQ ID NO:96); a fifth probe comprising the sequence TTTTTTTTTTTTTCTCCTGCTTAGT (SEQ ID NO: 100); a sixth probe comprising the sequence TTTTTTTTTTTTAATGATGCACGTC (SEQ ID NO: 116); a seventh probe comprising the sequence TTTTTTAATTTCTAGCTACAGAGAAAT (SEQ ID NO:86); an eighth probe comprising the sequence TTTTTTTTTTCAAAGTGCTGGCCTC (SEQ ID NO:118); a ninth probe comprising the sequence TTTTTTTTTAATCAAAACATCTCCG (SEQ ID NO:127); a tenth probe comprising the sequence TTTTTTTTAATCAAGACATCTCCGA (SEQ ID NO:137); an eleventh probe comprising the sequence TTTTTTTTTTGAGATGCATGATGAG (SEQ ID NO:353); a twelfth probe comprising the sequence TTTTTTTTTTTACCAGGAGGCTGCC (SEQ ID NO:468); a thirteenth probe comprising the sequence TTTTTTTTTTTACCAGGAGGGAGCC (SEQ ID NO:472); a fourteenth probe comprising the sequence TTTTTTTTTTAGGCCATCTTGGA (SEQ ID NO:424); a fifteenth probe comprising the sequence TTTTTTTTTTTGTGATGGCCGGCGT (SEQ ID NO:436); a sixteenth probe comprising the sequence TTTTTTTTTTTAACCCCCATCACGT (SEQ ID NO:442); a seventeenth probe comprising the sequence TTTTTTTTTTTCGTGGACGGTAACC (SEQ ID NO:454); an eighteenth probe comprising the sequence TTTTTTTAAAAAAGCGGGCCAAACT (SEQ ID NO:156); a nineteenth probe comprising the sequence TTTTTTTTACGTCTGTAGGGAAGTA (SEQ ID NO:379); a twentieth probe comprising the sequence TTTTTAAATTTACCCAGAATCAGAA (SEQ ID NO:388); a twenty-first probe comprising the sequence TTTTTTTTTATACGTGATGTCTTAC (SEQ ID NO:405); a twenty-second probe comprising the sequence TTTTTTTTTTCCTAAAGTGTACCGC (SEQ ID NO:164); a twenty-third probe comprising the sequence TTTTTTTTTTTTTATTGTACTTGTACCGCC (SEQ ID NO:176); a twenty-fourth probe comprising the sequence TTTTTTTTTTTTTTTCAACCAAGTGTACCG (SEQ ID NO:188); a twenty-fifth probe comprising the sequence TTTTTTTTTTTTTTACAGCCAAGTGTACCG (SEQ ID NO:200); a twenty-sixth probe comprising the sequence TTTTTTTTTTTCACCGAAAGCTGGAGTCCC (SEQ ID NO:210); a twenty-seventh probe comprising the sequence TTTTTTTTTTTTCCGAAAGATGATTTT (SEQ ID NO:212); a twenty-eighth probe comprising the sequence TTTTTTTTTTTTCTGGTTGGAGCTGGAGTCCC (SEQ ID NO:224); a twenty-ninth probe comprising the sequence TTTTTTTTTTTGGTTGGAGATGATTTTT (SEQ ID NO:236); a thirtieth probe comprising the sequence TTTTTTTTTTTGATGTAAAGATCCACTGTG (SEQ ID NO:248); a thirty-first probe comprising the sequence TTTTTTTTTATGATGTAAAGGAGGATCC (SEQ ID NO:260); a thirty-second probe comprising the sequence TTTTTTTTTGTATCTCTCAAGAGGATCCAA (SEQ ID NO:484); a thirty-third probe comprising the sequence TTATTATTAAGAGTTAAAAAGGAGGATC (SEQ ID NO:811); a thirty-fourth probe comprising the sequence TATTATTATGTGCACAAACAGGAGGATC (SEQ ID NO:506); a thirty-fifth probe comprising the sequence TTTTTTTTTTAGGGCCCAGACACTAACAGC (SEQ ID NO:268); a thirty-sixth probe comprising the sequence TTTTTTTTTTAAATTAAAGATCAGTTTC (SEQ ID NO:276); (b) a plurality of primer pairs, the plurality of primer pairs comprising a first primer pair comprising the sequences GTACTGGTGGAGTATTTGATAGTG (SEQ ID NO:1) and CGTCAAGGCACTCTTGCCTAC (SEQ ID NO:2); a second primer pair comprising the sequences CAATTTCTACAAGAGATCCTCTCTCT (SEQ ID NO:5) and CTCCATTTTAGCACTTACCTGTGAC (SEQ ID NO:6); a third primer pair comprising the sequences ACCCTAGCCTTAGATAAAACTGAGC (SEQ ID NO:7) and TTTGTTGTCCAGCCACCATGA (SEQ ID NO:8); a fourth primer pair comprising the sequences ATAGCCTCAATTCTTACCATCCACAAAATG (SEQ ID NO: 9) and CAGATATATTTCTTCATGAAGACCTCACAGTAA (SEQ ID NO:10); a fifth primer pair comprising the sequences CTTGTGGAGCCTCTTACACCC (SEQ ID NO: 11) and TGCCGAACGCACCGGA (SEQ ID NO:12); a sixth primer pair comprising the sequences GCCAGTTAACGTCTTCCTTCTC (SEQ ID NO: 13) and ATCGAGGATTTCCTTGTTGGCTT (SEQ ID NO:14); a seventh primer pair comprising the sequences CCTCCACCGTGCAGATCATC (SEQ ID NO:15) and TTCCCTGATTACCTTTGCGAT (SEQ ID NO:16); an eighth primer pair comprising the sequences CCACACTGACGTGCCTCT (SEQ ID NO:511) and GCACACGTAGGGGTTGTCCAAGA (SEQ ID NO:512); a ninth primer pair comprising the sequences CCACACTGACGTGCCTCT (SEQ ID NO: 513) and GTACACGCTGGCCACGCCG (SEQ ID NO:514); a tenth primer pair comprising the sequences CCACACTGACGTGCCTCT (SEQ ID NO:515) and CAGGCGGCACACGTGAT (SEQ ID NO:516); an eleventh primer pair comprising the sequences CCACACTGACGTGCCTCT (SEQ ID NO:517) and AGGCGGCACACGTGCGGGTTAC (SEQ ID NO:518); a twelfth primer pair comprising the sequences GGAGGACCGTCGCTTGG (SEQ ID NO:17) and TCTTTCTCTTCCGCACCCAG (SEQ ID NO:18); a thirteenth primer pair comprising the sequences GAGGGTCTGACGGGTAGAGTG (SEQ ID NO:380) and TGGCCGCCAGGTCTTGATGTA (SEQ ID NO:381), a fourteenth primer pair comprising the sequences CTTGATGAGCAGCAGCGAAA (SEQ ID NO:397) and CCTTCAGTTCTCCCACCTTCTG (SEQ ID NO:398); a fifteenth primer pair comprising the sequences ATGGCTGTGGTTTGTGATGGT (SEQ ID NO:414) and ACACCAGCCATCACGTAAGACA (SEQ ID NO:415); a sixteenth primer pair comprising the sequences AGTTGGGGTTGTAGTCGGTCAT (SEQ ID NO:363) and TATGGAGCAAAACTACTGTAGAGCC (SEQ ID NO:357); a seventeenth primer pair comprising the sequences AGTTGGGGTTGTAGTCGGTCAT (SEQ ID NO:363) and CCAGCTACATCACACACCTTGACT (SEQ ID NO:358); an eighteenth primer pair comprising the sequences AGTTGGGGTTGTAGTCGGTCAT (SEQ ID NO:363) and TAATACCAAAAGTTACCAAAACTGCA (SEQ ID NO:359); a nineteenth primer pair comprising the sequences GGAGTGCCATCGCTGTTTGAAATGAGCAGGCACT (SEQ ID NO:19); a twentieth primer pair comprising the sequences GGAGTGCCATCGCTGTTTGAAATGAGCAGGCACT (SEQ ID NO:19); a twenty-first primer pair comprising the sequences TACAGCCCTGGATATTCTTAGTAGCGC (SEQ ID NO:20); a twenty-second primer pair comprising the sequences TACAGCCCTGGATATTCTTAGTAGCGC (SEQ ID NO:20); a twenty-third primer pair comprising the sequences GTGATCGCACAGTAGGACAGCGGCTGCGATC (SEQ ID NO:26) and TTTCTGGTGCTATGAGGAAATGACCAACCACCAGA (SEQ ID NO: 23); a twenty-fourth primer pair comprising the sequences CTCTAGGAGATATCATTCCAAATTCGCCTTCTCCTAG (SEQ ID NO:27) and TTTCTGGTGCTATGAGGAAATGACCAACCACCAGA (SEQ ID NO:23); a twenty-fifth primer pair comprising the sequences CTCTAGGAGATATCATTCCAAATTCGCCTTCTCCTAG (SEQ ID NO:27) and AAGGAGTTAGCAGCATGTCAGC (SEQ ID NO:519); a twenty-sixth primer pair comprising the sequences CTCTAGGAGATATCATTCCAAATTCGCCTTCTCCTAG (SEQ ID NO:27) and AACTTCAGACTTTACACAACCTGC (SEQ ID NO:520); a twenty-seventh primer pair comprising the sequences CTCTAGGAGATATCATTCCAAATTCGCCTTCTCCTAG (SEQ ID NO:27) and ATTGATTCTGATGACACCGGA (SEQ ID NO:521); a twenty-eighth primer pair comprising the sequences GGACGAAAATCCAGACCCCAAAAGGTGTTTCGT (SEQ ID NO:32) and AGAAGACGTGACAGGAACTGGAGGACCCGTCTT (SEQ ID NO:30); a twenty-ninth primer pair comprising the sequences GACAGTATTTTGCAGTAATGGACTGGATATATCAGA (SEQ ID NO:29) and GAATTTCACAGGATTGATTGCTGGTGTTGTCTC (SEQ ID NO:28); and (c) a plurality of blocking nucleic acids, the plurality of blocking nucleic acids comprising a first blocking nucleic acid comprising the sequence TACGCCACCAGCT(invdT)ₙ, wherein n is 1, 2, or 3 (SEQ ID NO:281); a second blocking nucleic acid comprising the sequence TCTCTGAAATCACTGAGCAGG(invdT)ₙ, wherein n is 1, 2, or 3 (SEQ ID NO:293); a third blocking nucleic acid comprising the sequence CACCATGATGTGCAT(invdT)ₙ, wherein n is 1, 2, or 3 (SEQ ID NO:296); a fourth blocking nucleic acid comprising the sequence GAGAITTCACTGTAGC(invdT)ₙ, wherein n is 1, 2, or 3 (SEQ ID NO:303); a fifth blocking nucleic acid comprising the sequence GAGCCCAGCAC (invdT)ₙ, wherein n is 1, 2, or 3 (SEQ ID NO:308); a sixth blocking nucleic acid comprising the sequence CGGAGATGTTGCTTCTCTTAATTCC(invdT)ₙ, wherein n is 1, 2, or 3 (SEQ ID NO:311); a seventh blocking nucleic acid comprising the sequence CATCACGCAGCTCATG(invdT)ₙ, wherein n is 1, 2, or 3 (SEQ ID NO:316); an eighth blocking nucleic acid comprising the sequence CCAGCAGTTTGGCCAGCCCT(invdT)ₙ, wherein n is 1, 2, or 3 (SEQ ID NO:321); a ninth blocking nucleic acid comprising the sequence TGTACTCCCCTACA (invdT)ₙ, wherein n is 1, 2, or 3 (SEQ ID NO:382); and a tenth blocking nucleic acid comprising the sequence TCTGCTTCTGGGTAAG (invdT)ₙ, wherein n is 1, 2, or 3 (SEQ ID NO:399).

In some embodiments, the kit comprises a microcarrier with an identifier corresponding to a positive control and to which a probe specific for a positive control gene sequence is coupled, and a primer pair specific for the positive control DNA sequence. For example, in some embodiments, a positive control RNA sequence comprises a sequence of a human hypoxanthine phosphoribosyltransferase 1 (HPRT1) gene. In some embodiments, the primer pair specific for the positive control RNA sequence comprises the sequences GGAAGATATAATTGACACTGGCAAAACA (SEQ ID NO:34) and ATTCATTATAGTCAAGGGCATATCC (SEQ ID NO:35). In some embodiments, each probe is coupled to a microcarrier of the present disclosure with a unique identifier. In some embodiments, the kit comprises a microcarrier of the present disclosure with an identifier corresponding to a negative control, e.g., with a probe that does not hybridize with amplified DNA.

In some embodiments, the kit comprises a primer pair, with one or both primers of the pair labeled with a detection reagent, e.g., as described supra. In some embodiments, the detection reagent comprises a fluorescent detection reagent. In some embodiments, the detection reagent comprises biotin, and the kit comprises streptavidin conjugated to a signal-emitting entity (e.g., streptavidin conjugated to phycoerythrin).

In some embodiments, the kits or articles of manufacture may further include one or more detection reagents of the present disclosure for detecting an amount of the first analyte bound to the first microcarrier and an amount of the second analyte bound to the second microcarrier. In some embodiments, the detection reagent for the first analyte may be the same as the detection reagent for the second analyte. In other embodiments, the detection reagent for the first analyte may be different from the detection reagent for the second analyte.

In some embodiments, the kits or articles of manufacture may further include instructions for using the kit or articles of manufacture to detect one or more DNA or RNA mutations of the present disclosure. These instructions may be for using the kit or article of manufacture, e.g., in any of the methods described herein.

In some embodiments, the kits or articles of manufacture may further include one or more detection reagents (e.g., as described above, such as streptavidin conjugated to PE), along with any instructions or reagents suitable for coupling a detection reagent to one or more analytes, or for coupling a detection reagent to one or more macromolecules that recognize an analyte. The kits or articles of manufacture may further include any additional components for using the microcarriers in an assay (e.g., a multiplex assay), including without limitation a plate (e.g., a 96-well or other similar microplate), dish, microscope slide, or other suitable assay container; a non-transitory computer-readable storage medium (e.g., containing software and/or other instructions for analog shape or code recognition); washing agents; buffers; plate sealers; mixing containers; diluents or storage solutions; and the like.

In some embodiments, a probe of the present disclosure may be coupled to a microcarrier of the present disclosure, e.g., a microcarrier described herein and/or a microcarrier produced by any of the methods described herein. Any of the probes described herein may find use in the methods and/or microcarriers of the present disclosure.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Multiplex Detection of Lung Cancer-Associated Mutations Using Encoded Microcarriers

As described above, multiplex screening for cancer-associated DNA mutations and RNA variants using DNA and RNA isolated from a blood or tissue sample represents a non-invasive method for early detection. The following Example describes the validation of a microcarrier-based approach for multiplex screening to identify a variety of important DNA mutations and RNA variants associated with lung cancer.

Materials and Methods

### Sample Preparation

A flowchart of the method used to test the detection of DNA mutations and RNA variants using encoded microcarriers is provided in FIG. 3. DNA was isolated from the plasmids described in Table C or K562 cells as described in Table B using standard techniques. DNA was quantified using a NanoDrop™ UV-Vis spectrophotometer (Thermo Scientific) or Qubit® Fluorometer (Thermo Scientific) according to manufacturer's instructions. Concentration of extracted DNA used for all experiments was ≥12.5 ng/μL. To test the detection of RNA variants using encoded microcarriers, RNA was isolated from HEK293 cells transfected with the plasmids described in Table D after 24 h. culturing using standard techniques.

The mutations that were detected in these experiments are provided in Tables A1 & A2.

For the limit of detection (LOD) testing of DNA mutations, DNA was extracted from K562 cells for use as “wild type” DNA. DNA with one of various mutations of interest was obtained using plasmids bearing mutated KRAS, NRAS, PIK3CA, BRAF, EGFR, AKT1, MEK1, or HER2 sequences. Wild type and mutated DNA were mixed and diluted to achieve a total DNA concentration of 12.5 ng/μL with a ratio of 1% mutated DNA to 99% wild type DNA. Concentration of each DNA sample used for the experiments is shown in Tables B and C below.

For the limit of detection (LOD) testing for RNA variants, RNA was obtained using plasmids bearing sequences of RNA variants of ALK, ROS, RET NTRK1 or cMET.

TABLE B
Wild-type DNA.

| | WT | | | | | | | |
|---|---|---|---|---|---|---|---|---|
| | WT DNA Stock | | WT DNA 500 ng/μl | | | WT DNA 100 ng/μl | | |
| Cell | mutation | conc | stock | final | | 100 ng/μl | final | |
| line | % | (ng/μl) | volume | volume | Tris | volume | volume | Tris |
| K562 | — | 1208 | 207.0 | 500 | 293.0 | 100 | 1000 | 900 |

### Polymerase Chain Reaction (PCR)

Mutation-enriching PCR was used to selectively amplify polynucleotides having a mutation of interest from the DNA samples prepared above. Locked nucleic acids (LNAs) with 3′ inverted dT nucleotides were used to block the amplification of wild-type DNA sequences, thereby enriching for mutations of interest. Briefly, a blocking nucleic acid was included in the PCR reaction to block amplification of the wild-type locus. The blocking nucleic acid hybridized to the sense strand of the wild-type locus and prevented primer extension to amplify from the sense strand, as shown in FIG. 4. LNAs were used because of more stable hybridization, as compared to oligonucleotides with standard nucleic acids. Any copies of the mutant locus present in the sample could be amplified by PCR with no interference from the blocking nucleic acid, since it did not hybridize with the mutant sequence (FIG. 5). The following primer pairs were used: SEQ ID NOs: 1 and 2, 3 and 4, 5 and 6, 7 and 8, 9 and 10, 11 and 12, 13 and 14, 15 and 16, 17 and 18. The following blocking nucleic acids were used: SEQ ID NOs: 281, 286, 293, 296, 303, 308, 311, 316, 321, and 365.

Each sample was vortex-mixed, then spun down and kept on ice. PCR reactions were carried out as follows. For each sample, two PCR reactions were performed according to the amounts listed in Tables D and E. Each PCR reaction included 4 μL of 12.5 ng/μL extracted DNA, for a total of 50 ng DNA. Once PCR reaction mixes were generated in PCR tubes, the tubes were mixed by tapping, spun down briefly, and placed in a thermocycler. PCR thermocycle conditions were as described in Table F below. The ramp rate was 1° C./second.

To selectively amplify polynucleotides having a RNA variant of interest from the RNA samples prepared above, cDNA was first generated from the isolated RNA by reverse transcription polymerase chain reaction (RT-PCR), which was further amplified by mutation-enriching PCR. PCR thermocycle conditions were as described in Table G below. The ramp rate was 5° C./second. The following primers were used: SEQ ID NOs: 19-37.

TABLE D
PCR reaction 1.
PCR Reaction 1

| Material | Vol. (μL) per reaction |
|---|---|
| Reaction Mix 1 | 11 |
| Primer Mix 1 | 5 |
| Extracted DNA/PC/NC | 4 |
| Total | 20 |

TABLE E
PCR reaction 2.
PCR Reaction 2

| Material | Vol. (μL) per reaction |
|---|---|
| Reaction Mix 2 | 11 |
| Primer Mix 2 | 5 |
| Extracted DNA/PC/NC | 4 |
| Total | 20 |

TABLE F
PCR conditions for DNA mutations.

| Step | Temp | Time | | Cycle | Ramp rate |
|---|---|---|---|---|---|
| Pre-denature hold | 95° C. | 5 | min | 1 | 1° C./s |
| Denature | 95° C. | 20 | sec | 38 cycles | |
| LNA Blocking | 70° C. | 20 | sec | | |
| Annealing | 55° C. | 20 | sec | | |
| Extension | 60° C. | 25 | sec | | |

| | | |
|---|---|---|
| Storage | 4° C. | ∞ |

TABLE G
PCR conditions for RNA variants.

| Step | Temp | Time | | Cycle | Ramp rate |
|---|---|---|---|---|---|
| cDNA synthesis (RT) | 55° C. | 15 | min | 1 | 5° C./s |
| Pre-denature hold | 95° C. | 2 | min | 1 | |
| Denature | 95° C. | 15 | sec | 45 | |
| Annealing | 60° C. | 30 | sec | | |
| Extension | 72° C. | 30 | sec | | |

| | | | |
|---|---|---|---|
| Storage | 4° C. | ∞ | ∞ |

### Hybridization

PCR amplicons were hybridized to the capture agent probe sequences of the microcarriers. The probes were designed to hybridize with the mutant sequence and not the wild-type sequence, thereby allowing the specific detection of the mutant DNA or RNA variant. Combined with the use of blocking nucleic acids that bind the wild-type sequence, this strategy enables the assay to detect mutant DNA even when present at a much lower copy number than the corresponding wild-type locus (see FIGS. 4 & 5). The following probes were used for DNA: SEQ ID NOs: 45, 53, 63, 75, 86, 96, 100, 116, 118, 127, 137, 353, and 156. The following probes were used for RNA: SEQ ID NOs: 164, 176 188, 200, 210, 212, 224, 236, 248, 260, 268, and 276.

Briefly, encoded microcarriers, each individually specific for a mutation or RNA variant shown in Tables A1 & A2, were pooled into a single well of a 96-well plate. The microcarrier solution was mixed by vortexing for 10 seconds, then 20 μL of microcarrier solution was added to each well of a 96-well plate. The stock microcarrier solution was re-vortexed every 4 wells to ensure a homogeneous suspension. 100 μL hybridization buffer (5×SSPE buffer) was added to each well. PCR products were spun down and denatured by heating to 95° C. for 5 minutes, then cooled down to 4° C. 10 μL of denatured PCR product 1 or 10 μL of denatured PCR product 2 was added to each well. The assay plate was mixed by shaking at 1200 rpm for 20 minutes at 37° C. Wells were then washed with 1× wash buffer.

For detection, 50 μL of streptavidin-conjugated phycoerythrin (SA-PE) solution was added to each well, and plates were again shaken at 1200 rpm for 10 minutes at 37° C. Wells were then washed again. Each well was then subject to analog image decoding and fluorescent detection.

Results

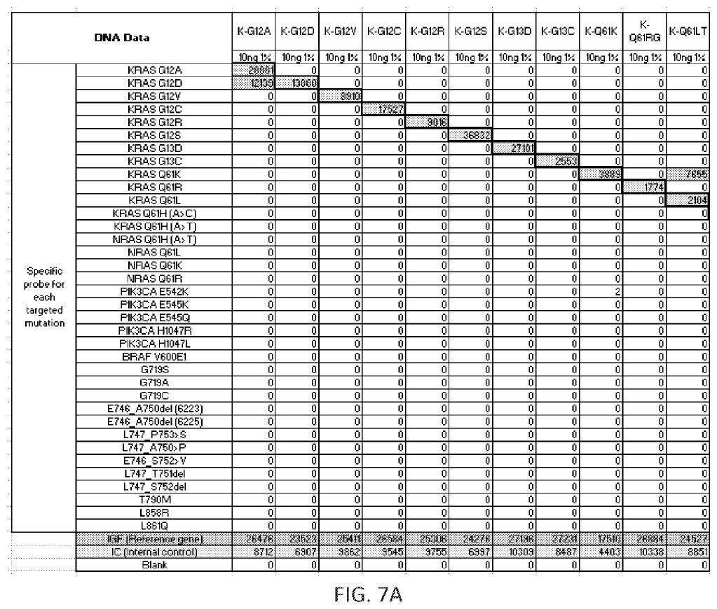

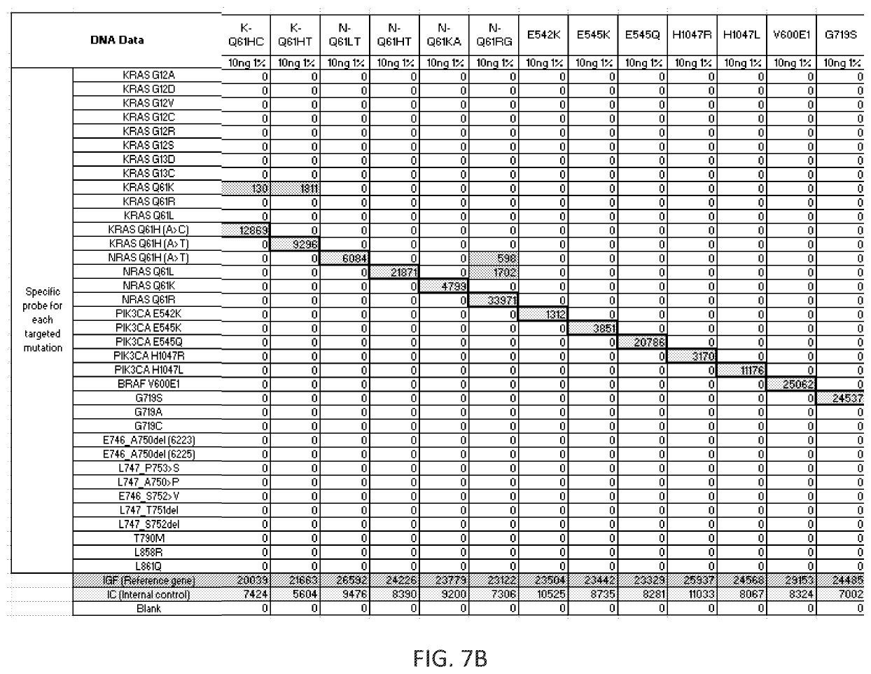

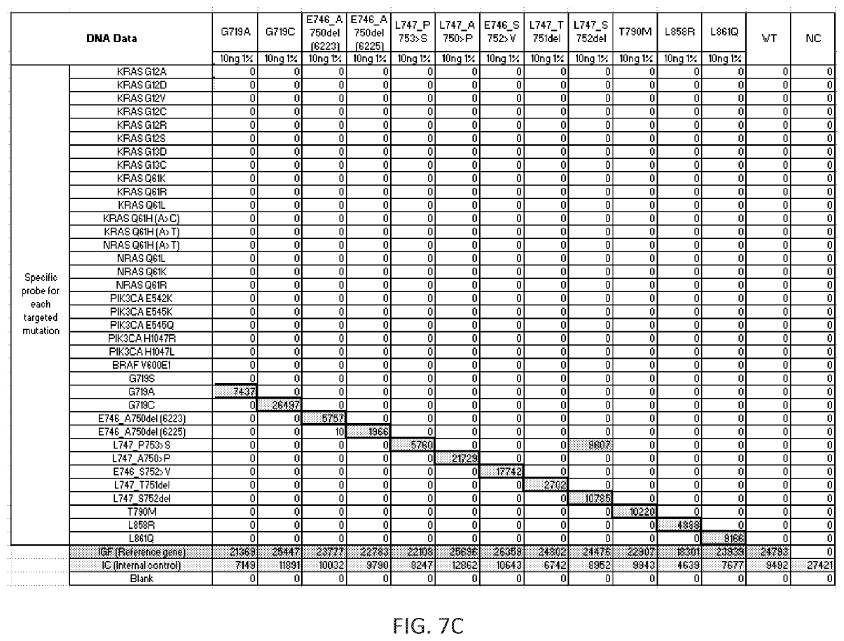

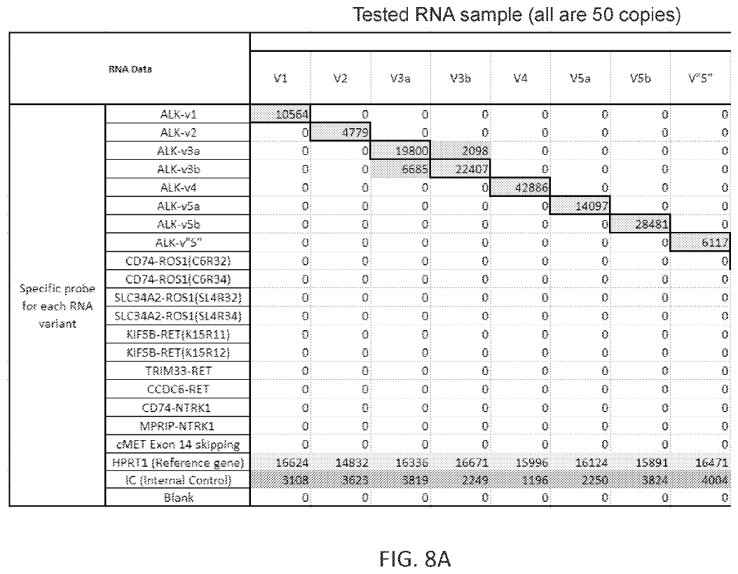

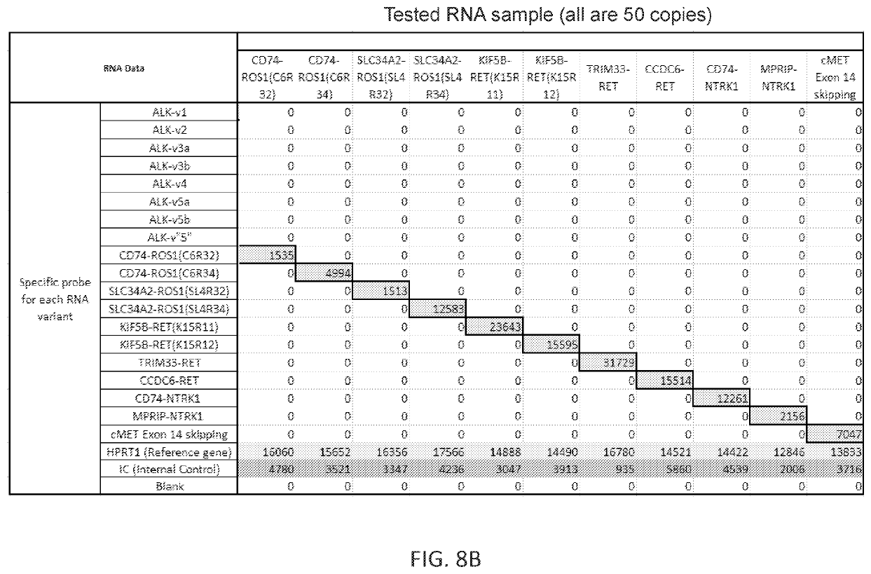

Microcarriers with probes specific for each of the mutations or RNA variant listed in Table A were used to detect the presence of mutated DNA or RNA variants in the LOD testing assays described above. The results of these experiments are shown in FIGS. 7A-7C (DNA) and FIGS. 8A & 8B (RNA). The microcarrier-based assay was validated for each mutation in a pairwise fashion, using one probe and one mutated DNA sequence or one RNA variant sequence per assay. The columns indicate which mutated DNA sequence or RNA variant sequence was present in the sample. The rows indicate which probe was coupled to the microcarriers for these assays. For DNA mutations, as a negative control, “blank” microcarriers with no probe were used. For a positive control DNA, human IGF DNA was amplified and detected using a probe specific for the amplified sequence.

FIGS. 7A-7C report the fluorescence signal (in arbitrary units, AU) obtained for each DNA experiment. As shown, in nearly all cases in which the mutated DNA sequence and the probe were mismatched, no fluorescence was detected, indicating a lack of hybridization between the probe and DNA. KRAS DNA weakly cross-reacted with the KRAS G12D probe, and the NRAS Q61L probe, but yielded much lower fluorescence signal than each respective KRAS or NRAS DNA sample. In contrast, when each mutated DNA sequence was mixed with the appropriate microcarrier-coupled probe, hybridization was detected by a strong fluorescence signal.

No signal other than the positive control was detected using wild-type DNA, indicating that the LNA oligonucleotides were effective in blocking wild-type DNA amplification and/or the probe sequences were effective in eliminating hybridization of wild-type DNA.

For RNA variant detection, as a negative control, “blank” microcarriers with no probe were used. For a positive control, human HPRT1 RNA was reverse transcribed, amplified and detected using a probe specific for the amplified sequence.

FIGS. 8A & 8B report the fluorescence signal (in arbitrary units, AU) obtained for each experiment. As shown, in nearly all cases in which the cDNA sequence for the RNA variant and the probe were mismatched, no fluorescence was detected, indicating a lack of hybridization between the probe and cDNA. The cDNA of ALK-v3a weakly cross-reacted with the ALK-v3b probe, and the cDNA of ALK-v3b weakly cross-reacted with the ALK-v3a probe, but yielded much lower fluorescence signal than each respective variants. This was likely due to the sequence similarity between the two variants. In contrast, when each cDNA sequence of the RNA variant was mixed with the appropriate microcarrier-coupled probe, hybridization was detected by a strong fluorescence signal.

These results demonstrate the sensitive and accurate detection of individual mutations of interest even with wild-type DNA present in amounts greater by orders of magnitude than the mutated sequences of interest. Advantageously, multiplex detection of several mutations in tandem leads to greater confidence and fidelity, since detection of each gene acts as an internal control for all of the other genes. These results suggest a rapid, multiplexed strategy using encoded microcarriers with oligonucleotide probes for the identification of important cancer-associated mutations from human samples.

### Example 2: Multiplex Detection of Lung Cancer-Associated Mutations from Blood Samples Using Encoded Microcarriers

The preceding Example demonstrated the use of encoded microcarriers with oligonucleotide probes for multiplex detection of cancer-associated mutations based on isolated DNA or RNA samples. The following Example demonstrates the efficacy of this approach using DNA and RNA extracted from blood samples.

Materials and Methods

Isolation of Plasma Total RNA and Plasma Cell-Free DNA (cfDNA)

FIG. 6 shows the process of isolating plasma total RNA and plasma cfDNA from a blood sample (see also Best, M. G. et al. (2015) Cancer Cell 28:666-676). To isolate plasma total RNA, whole blood was collected in purple-capped BD Vacutainers or cell-free DNA BCT containing EDTA anti-coagulant. Tubes were stored at room temperature and processed within 1 hour. The cells and aggregates were removed by centrifugation at room temperature for 20 mins at 200 g, resulting in total RNA-rich plasma. The total RNA-rich plasma was transferred to a 15 ml conical tube (tube A) without pipetting the supernatant or touching the red blood cells with the pipet tip. The total RNA-rich plasma was centrifuged for 20 minutes at 100 g at room temperature, and the supernatant was transferred to a second conical tube (tube B). The supernatant in tube B was further centrifuged for 20 minutes at 360 g at room temperature. The supernatant was removed and transferred to a third conical tube (tube C). 30 μL RNA stable buffer was added to the total RNA-rich pellet in tube B and incubated overnight at 4° C. before being frozen down at −80° C. for future use.

The supernatant in tube C was aliquoted into 1.5 mL or 2 mL tubes and centrifuged at room temperature for 10 minutes at 16,000 g. The supernatant containing cfDNA from each tube was transferred to a 15 mL conical tube. For extraction of cfDNA, the following commercial kits were used according to manufacturer's protocols: Promega-Maxwell RSC ccfDNA Plasma Kit, Thermo-MagMAX Nucleic Acid Isolation Kit, Catchgene-Catch-cfDNA Serum/Plasma Ki, Qiagen-QIAamp Circulating Nucleic Acid Kit.

Isolation of cfDNA-Rich Plasma

To isolate cfDNA-rich plasma only, whole blood was centrifuged at 1,600 g for 10 minutes. The upper plasma was transferred to 1.5 nL or 2 mL tubes and centrifuged at 16,000 g for 10 minutes. The resulting upper plasma was transferred to a 15 mL conical tube and frozen at −80° C. for future use.

### Extraction of Plasma Total RNA

The total-RNA rich sample was defrosted for 5-10 minutes at 4° C., dissolved in 1 mL RNA Isolation Buffer and left at room temperature for 10 minutes under the fume hood. 200 μL chloroform was added to each tube, followed by 15-30 seconds of vortexing and left for 2-3 minutes at room temperature. The tubes were then centrifuged for 15 minutes at 12,000 g at 4° C. The aqueous phase (upper phase) which contained the RNA Isolation Buffer and chloroform mix was then transferred to a different tube. To minimize carryover of contaminating DNA, about 3-4 mm of the aqueous phase above the interphase was not transferred. 500 μL of isopropanol (cooled to −20° C.) was added to each tube with the RNA isolation buffer and chloroform mix. The tubes were inverted to mix and left at −20° C. for an hour. The samples were then centrifuged for 15 minutes at 12,000 g at 4° C., all liquids were removed and 1 mL of freshly made RNA Wash Buffer was added. Upon centrifugation at 7,500 g for 10 minutes at 4° C., all liquids were removed and the pellets were dried by placing the tubes in a fume hood for 5 minutes with the lids open. When the RNA pellets appeared dry, 40 μL RNA Elution Buffer was added to dissolve the RNA and the solution was vortexed and kept on ice.

PCR reactions were generated using the DNA isolated above according to the following proportions. Primer mixes were generated as described in Example 1.

TABLE G
PCR reaction 1.
PCR Reaction 1

| Material | Vol. (μL) per reaction |
|---|---|
| Reaction Mix 1 | 10 |
| Primer Mix 1 | 10 |
| Extracted DNA/PC/NC | 20 |
| Total | 40 |

TABLE H
PCR reaction 2.
PCR Reaction 2

| Material | Vol. (μL) per reaction |
|---|---|
| Reaction Mix 2 | 10 |
| Primer Mix 2 | 10 |
| Extracted DNA/PC/NC | 20 |
| Total | 40 |

Other PCR conditions (e.g., thermocycling), hybridization conditions, and detection were as described in Example 1.

### Results

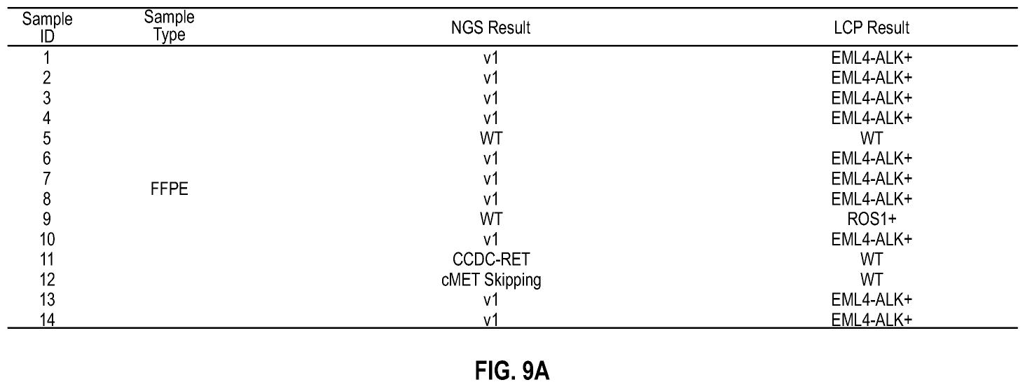

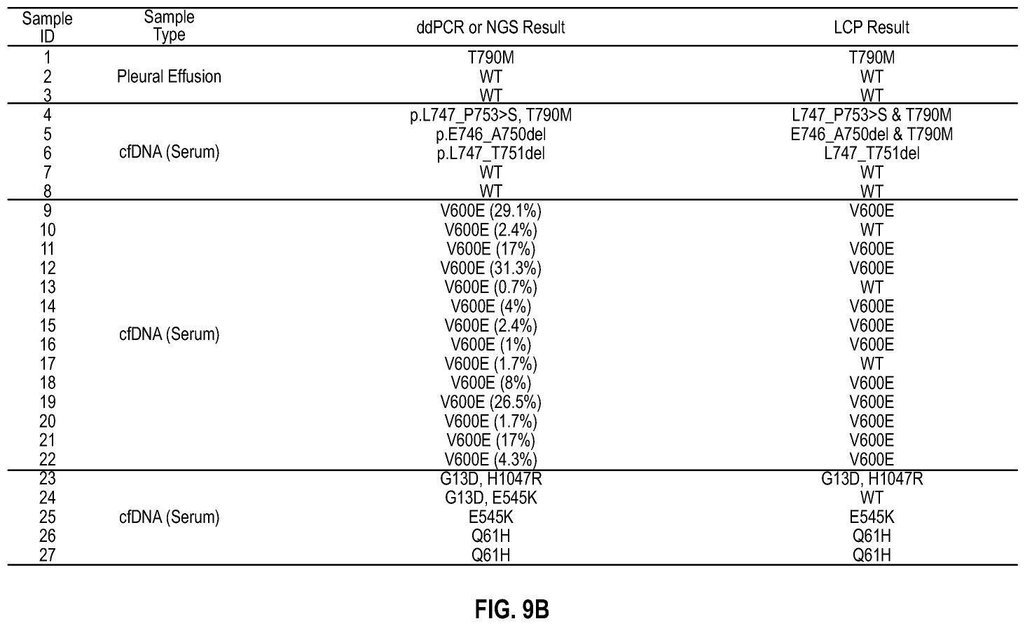

In FIG. 9A, RNA was obtained from formalin-fixed, paraffin-embedded (FFPE) samples, and selected mutations were detected by next-generation sequencing (NGS), as compared to the microcarrier approach described herein (LCP). In FIG. 9B, DNA was obtained from pleural effusion or cfDNA in serum samples, and selected mutations were detected by ddPCR or next-generation sequencing (NGS), as compared to the microcarrier approach described herein (LCP). These results demonstrate the successful, multiplex detection of multiple cancer-associated mutations in DNA and RNA from patient samples using the methods described herein.

Next, the performance of the lung cancer panel (LCP) detected by the microcarrier approach described herein was compared to that of digital PCR (ddPCR). These approaches were compared using tissue samples (FPE samples) obtained from stage III non-small cell lung cancer patients. The ddPCR assay used for this comparison study only detected mutations in the EGFR gene.

9 samples were tested by ddPCR for mutations in the EGFR gene. The results are shown in Table 1. These results show that the LCP approach was able to detect the same mutations as the reference ddPCR method, while also correctly detecting wild-type (WT) EGFR genes as well.

TABLE I

| | | Lung Cancer | |
|---|---|---|---|
| | | Panel - FFPE | External 3rd Party |
| | | (DNA) | Single Gene |
| Ref. Method | Sample ID | EGFR | Testing Results** |
| Digital PCR | 1 | WT | WT |
| EGFR (only) | 2 | p.L747_T7521del | L747_T751del |
| | 3 | WT | WT |
| | 4 | p.L858R | L858R |
| | 5 | p.E746_A750del | E746_A750del |
| | 6 | p.L858R | L858R |
| | 7 | p.E746_A750del | E746_A750del |
| | 8 | WT | WT |
| | 9 | WT | WT |

The LCP panel was also compared with a RT-PCR-based approach to detect mutations in EGFR, KRAS, and BRAF genes from 41 patient samples as described above (Table J). These results confirm that the LCP approach was able to correctly identify particular mutations and wild-type sequences in these genes, as compared to an existing RT-PCR approach.

TABLE J

| | | | | | External 3rd Party |
|---|---|---|---|---|---|
| | | Lung Cancer Panel - FFPE (DNA) | | | RT-PCR |
| Ref. Method | Sample ID | EGFR | KRAS | BRAF | test Results |
| RT-PCR | 1 | WT | WT | V600E1 | BRAF |
| EGFR | 2 | WT | WT | V600E1 | BRAF |
| KRAS | 3 | WT | G12V | WT | KRAS |
| BRAF | 4 | WT | WT | WT | EGFR ex19 ¹ |
| | 5 | WT | WT | WT | KRAS 146 ¹ |
| | 6 | p.L747_P753 > S | WT | WT | EGFR ex19 |
| | 7 | WT | Q61K | WT | KRAS 61 |
| | 8 | WT | WT | WT | KRAS 146 ¹ |
| | 9 | L858R | WT | WT | EGFR L858R |
| | 10 | WT | WT | V600E1 | BRAF V600E1 |
| | 11 | WT | G12C | WT | KRAS G12C |
| | 12 | WT | G12V | WT | KRAS G12V |
| | 13 | WT | G12V | WT | KRAS G12V |
| | 14 | WT | G12C | WT | KRAS G12C |
| | 15 | p.E746_A750del | WT | WT | EGFR E746_A750del |
| | 16 | G719A | WT | WT | EGFR G719A |
| RT-PCR | 17 | WT | N/A | N/A | WT |
| EGFR only | 18 | WT | N/A | N/A | WT |
| | 19 | Exon 19 del (E746_A750 del) | N/A | N/A | Exon 19 del |
| | 20 | WT | N/A | N/A | WT |
| | 21 | WT | N/A | N/A | WT |
| | 22 | Exon 19 del (L747_P753 > S) | N/A | N/A | Exon 19 del |
| | 23 | WT | N/A | N/A | WT |
| | 24 | Exon 19 del (E746_A750 del) | N/A | N/A | Exon 19 del |
| | 25 | WT | N/A | N/A | WT |
| | 26 | Exon 19 del (L747_P753 > S) | N/A | N/A | Exon 19 del |
| | 27 | WT | N/A | N/A | Exon 20 Ins |
| | 28 | WT | N/A | N/A | WT |
| | 29 | WT | N/A | N/A | WT |
| RT-PCR | 30 | WT | N/A | N/A | WT |
| EGFR only | 31 | WT | N/A | N/A | Exon 19 mut ¹ |
| | 32 | WT | N/A | N/A | WT |
| | 33 | WT | N/A | N/A | WT |
| | 34 | WT | N/A | N/A | WT |
| | 35 | Exon 19 del | N/A | N/A | Exon 19 del |
| | 36 | L858R | N/A | N/A | L858R |
| | 37 | Exon 19 del | N/A | N/A | Exon 19 del |
| | 38 | WT | N/A | N/A | WT |
| | 39 | WT | N/A | N/A | WT |
| | 40 | WT | N/A | N/A | WT |
| | 41 | WT | N/A | N/A | WT |

¹ These targets were not included in the version of lung cancer panel used in this test, leading to discordant results.

The LCP panel was also compared with an NGS-based approach to detect mutations in EGFR, KRAS, NRAS, PIK3CA, and BRAF genes from 7 patient samples as described above (Table K). These results again confirm that the LCP approach was able to correctly identify particular mutations and wild-type sequences in these genes, as compared to NGS detection of mutations.

TABLE K

| | | | | | | | External |
|---|---|---|---|---|---|---|---|
| | | | | | | | 3rd Party |
| Ref. | Sample | Lung Cancer Panel - FFPE (DNA) | | | | | NGS Testing |
| Method | ID | EGFR | KRAS | NRAS | PIK3CA | BRAF | Results** |
| NGS | 53 | WT | WT | WT | WT | V600E | V600E |
| | 54 | WT | WT | WT | WT | WT | WT |
| | 55 | WT | WT | WT | WT | WT | WT |
| | 56 | WT | G12D (1833) | WT | WT | WT | WT |
| | 57 | WT | G12C | WT | WT | WT | G12C |
| | 58 | WT | WT | WT | WT | WT | WT |
| | 59 | WT | G12V | WT | WT | WT | G12V |

RNA detection of mutations using LCP as described above was also compared to NGS detection of RNA mutations. RNA was extracted from FFPE samples from 14 patients with stage III lung cancer and analyzed by NGS or the LCP approach for gene rearrangements in ALK, ROS1, RET, NTRK1, or cMET As shown in Table L, LCP was able to detect all of the mutations detected by NGS. In addition, LCP was able to identify rearrangements in RET and cMET that were not detectable by NGS, potentially due to the higher sensitivity of LCP (NGS detection limit was around 250 copies, while the detection limit of LCP was less than 10).

TABLE L

| | | | | | | | External |
|---|---|---|---|---|---|---|---|
| | | | | | | | 3rd Party |
| Ref. | Sample | Lung Cancer Panel - FFPE (RNA) | | | | | NGS Testing |
| Method | ID | ALK | ROS1 | RET | NTRK1 | cMET | Results** |
| NGS | 1 | v1 | WT | WT | WT | WT | EML4− |
| | | | | | | | ALK+ |
| | 2 | v1 | WT | WT | WT | WT | EML4− |
| | | | | | | | ALK+ |
| | 3 | v1 | WT | WT | WT | WT | EML4− |
| | | | | | | | ALK+ |
| | 4 | v1 | WT | WT | WT | WT | EML4− |
| | | | | | | | ALK+ |
| | 5 | WT | WT | WT | WT | WT | WT |
| | 6 | v1 | WT | WT | WT | WT | EML4− |
| | | | | | | | ALK+ |
| | 7 | v1 | WT | WT | WT | WT | EML4− |
| | | | | | | | ALK+ |
| | 8 | v1 | WT | WT | WT | WT | EML4− |
| | | | | | | | ALK+ |
| | 9 | WT | CD74- | WT | WT | WT | ROS1+ |
| | | | ROS1 | | | | |
| | 10 | v1 | WT | WT | WT | WT | EML4− |
| | | | | | | | ALK+ |
| | 11 | WT | WT | CCDC- | WT | WT | WT^{a} |
| | | | | RET | | | |
| | 12 | WT | WT | WT | WT | cMET exon 14 | WT^{a} |
| | | | | | | Skipping | |
| | 13 | v1 | WT | WT | WT | WT | EML4− |
| | | | | | | | ALK+ |
| | 14 | v1 | WT | WT | WT | WT | EML4− |
| | | | | | | | ALK+ |

^{a}The NGS detection limit on CCDC-RET and cMET exon 14 skipping variants was around 250 copies, which was inferior to those of the lung cancer panel (LoD < 10 copies).

To apply the LCP to liquid biopsy samples, LCP performance was tested against the ddPCR approach using either DNA extracted from pleural effusion, or cell-free DNA (cfDNA) from plasma/serum. Mutations in EGFR and BRAF were detected in 22 samples (Table M). Testing results using the ddPCR approach were provided by an external 3^{rd} party and reported the fractional abundance (FA %=II mutated copies/(# mutated copies+wild-type copies)) of each mutation. The ddPCR approach was used as a comparison because it is known as a method for absolute quantification of single-plexed mutations.

TABLE M

| Ref. | | | Lung Cancer Panel | External 3rd Party Single |
|---|---|---|---|---|
| Method | Sample Type | Sample ID | EGFR | Gene Testing Results** |
| Digital PCR | Pleural | 1 | T790M | EGFR T790M & L747_751 > P ^{a} |
| EGFR | Effusion | 2 | WT | WT |
| (only) | (DNA) | 3 | WT | WT |
| Digital PCR | cfDNA | 4 | p.L747_P753 > S, T790M | L747_P753 > S & T790M |
| EGFR | (Serum) | 5 | p.E746_A750del | E746_A750del & T790M |
| (only) | (DNA) | 6 | p.L747_T751del | L747_T751del |
| | | 7 | WT | WT |
| | | 8 | WT | WT |
| Digital PCR | cfDNA | 9 | V600E | V600E (29.1%) |
| BRAF | (Serum) | 10 | WT | V600E (2.4%) |
| (only) | (DNA) | 11 | V600E | V600E (17%) |
| | | 12 | V600E | V600E(31.3%) |
| | | 13 | WT | V600E (0.7%) |
| | | 14 | V600E | V600E (4%) |
| | | 15 | V600E | V600E (2.4%) |
| | | 16 | V600E | V600E (1%) |
| | | 17 | WT | V600E (1.7%) |
| | | 18 | V600E | V600E (8%) |
| | | 19 | V600E | V600E (26.5%) |
| | | 20 | V600E | V600E (1.7%) |
| | | 21 | V600E | V600E (17%) |
| | | 22 | V600E | V600E (4.3%) |

^{a} L747-751 > P is not a target in the lung cancer panel.

LCP performance using liquid biopsy samples was also compared against NGS using 5 patient samples for detection of mutations in EGFR, KRAS, NRAS, PIK3CA, and BRAF in cfDNA from serum (Table N).

TABLE N

| | | | | | | | | External |
|---|---|---|---|---|---|---|---|---|
| | | | Lung Cancer Panel - cfDNA | | | | | 3rd Party |
| Ref. | | Sample | (Serum) (DNA) | | | | | Single Gene |
| Method | Sample Type | ID | EGFR | KRAS | NRAS | PIK3CA | BRAF | Testing Results** |
| NGS | cfDNA (Serum) | 1 | WT | G13D | WT | H1047R | WT | KRAS G13D/PIK3CA |
| | (DNA) | | | | | | | H1047R |
| | | 2 | WT | WT | WT | WT | WT | KRAS G13D/PIK3CA |
| | | | | | | | | E545K |
| | | 3 | WT | WT | WT | E545K | WT | PIK3CA E545K |
| | | 4 | WT | Q61H | WT | WT | WT | KRAS Q61H |
| | | 5 | WT | Q61H | WT | WT | WT | KRAS Q61H |

Taken together, these results validate the use of LCP approach in detecting a variety of DNA and RNA mutations from both tissue samples and liquid biopsies, as compared to standard approaches.

### Example 3: Clinical Trial Data Demonstrating Multiplex Detection of Lung Cancer-Associated Mutations from Blood and Tissue Samples Using Encoded Microcarriers

The LCP approach described above was compared to other methods for detection of cancer-associated mutations, using liquid biopsies (blood samples) or tissue samples from patients with stage I or II lung cancer.

First, the LCP approach was compared with results obtained from the Oncomine Lung Cell-Free Total Nucleic Acid Research Assay (Cat. No. A35864; Thermo Fisher Scientific, Inc.), an NGS-based detection method. This assay is used to detect lung tumor-derived cell-free DNA and RNA (cell-free total nucleic acid; cfTNA) isolated from the plasma fraction of whole blood. 20 liquid biopsies (blood samples) were analyzed for lung cancer-associated mutations using either the Oncomine assay or the comprehensive lung cancer panel (LCP) approach described herein. As shown in FIG. 1A, the results from the 20 samples were nearly matching.

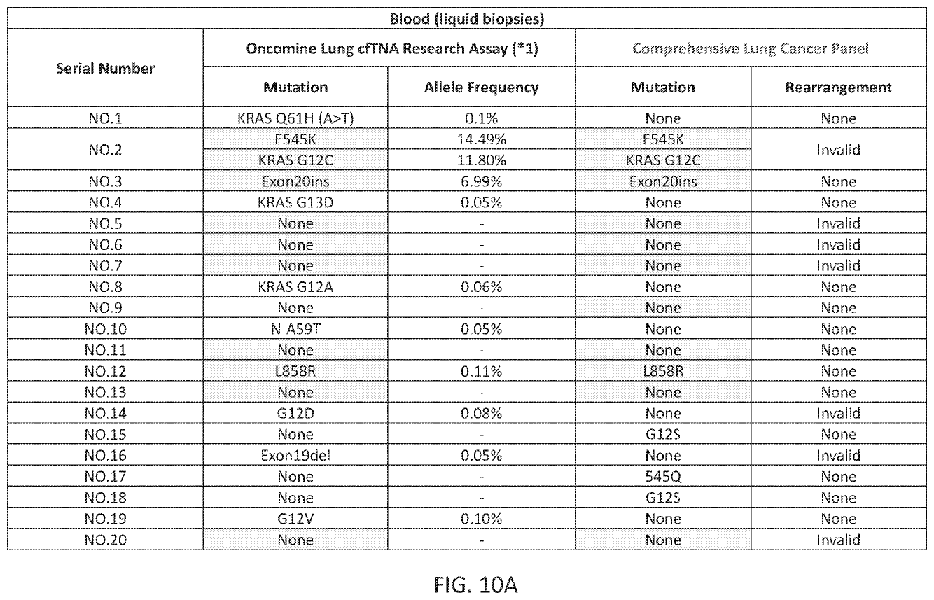

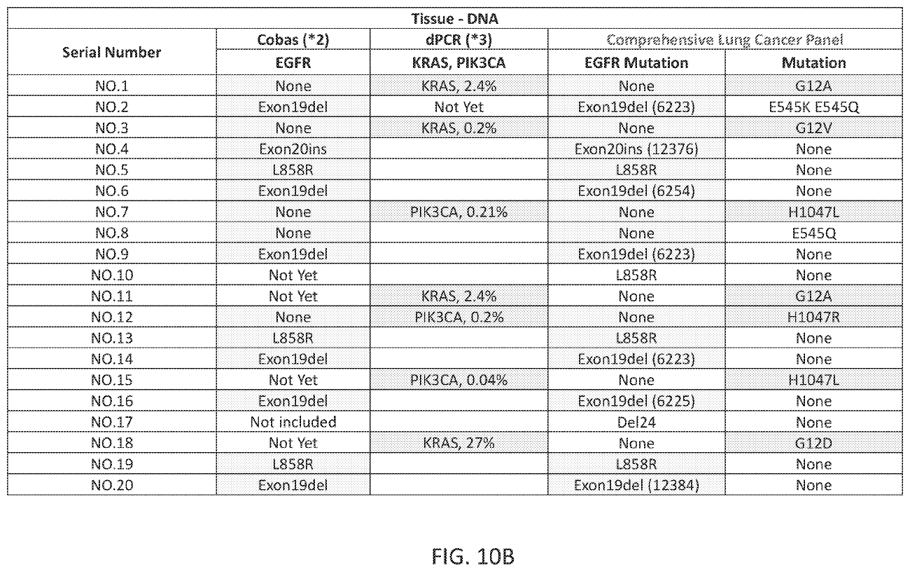

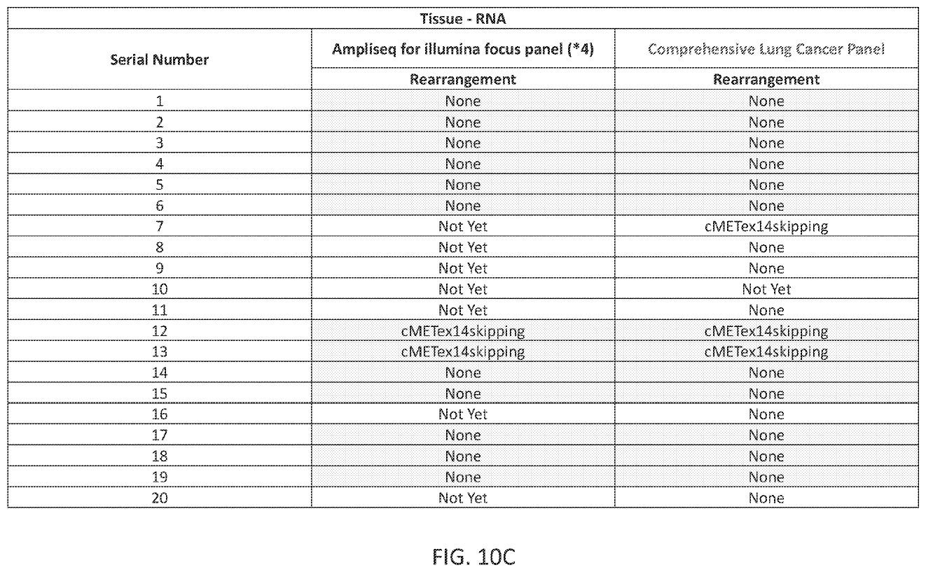

Second, the LCP approach was compared with results obtained from several PCR-based approaches for detecting mutations in EGFR, KRAS, or PIK3CA. For EGFR, the Cobas® EGFR Mutation Test v2 (Cat. No. 07248563190; Roche Molecular Diagnostics), a real-time PCR-based detection method, was used. The Cobas® EGFR Mutation Test v2 assay is used to identify 42 mutations in exons 18, 19, 20, and 21 of EGFR, including the T790M mutation. The results were also compared with the following digital PCR (dPCR)-based methods aimed at detection of mutations in KRAS or PIK3CA: the Bio-Rad® ddPCR™ KRAS G12/G13 screening kit (Cat. No. 1863506; Bio-Rad), the Bio-Rad® PrimePCR™ ddPCR™ PIK3CA mutation detection assay kit (Cat. No. 1863132; Bio-Rad) for the PIK3CA p.E545K mutation, the Bio-Rad® ddPCR™ mutation detection assays (Cat. No. 10042964; Bio-Rad), and the TaqMan™ Liquid Biopsy dPCR assay (Cat. No. A44177; Applied Biosystems/Thermo Fisher Scientific) for PIK3CA mutations. DNA was obtained from 20 tissue samples (formalin-fixed paraffin-embedded tumor tissue, FFPET) and used to detect lung cancer-associated mutations in EGFR, KRAS, or PIK3CA using the above PCR-based assays or the comprehensive lung cancer panel (LCP) approach described herein. As shown in FIG. 10B, the results from the 20 samples were nearly matching and successfully verified the feasibility of the LCP approach.

Finally, the LCP approach was compared with results obtained from the Illumina® AmpliSeq™ Focus Panel (Cat. No. 20019164; Illumina), an NGS-based, targeted resequencing assay for biomarker analysis of genes with known relevance to solid tumors. RNA was obtained from 20 tissue samples and used to detect lung cancer-associated mutations using the Illumina® AmpliSeq™ Focus Panel or the comprehensive lung cancer panel (LCP) approach described herein. As shown in FIG. 10C, the results from the 20 samples were nearly matching.

Taken together, these results validate the use of the LCP approach in detecting a variety of mutations from DNA and RNA using tissue samples or liquid biopsies, as compared to other NGS- or PCR-based approaches.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

TABLE A1
DNA mutations.

| | DNA Mutation | | | | |
|---|---|---|---|---|---|
| | | | | Amino Acid | Nucleotide |
| Frequency | Gene | Exon | Domain | Change | change |
| 15-25% | KRAS | Exon2 | GTP binding | G12D | c.34G > C (GGT > CGT) |
| | | | | G12V | c.34G > T (GGT > TGT) |
| | | | | G12C | c.35G > A (GGT > GAT) |
| 1% | NRAS | Exon3 | GTP binding | Q61L (A > T) | c.182A > T (CAA > CTA) |
| 1-3% | PIK3CA | Exon9 | Helical | E542K | c.1624G > A |
| | | | | E545K | c.1633G > A |
| | | Exon20 | Kinase | H1047R | c.3140A > G |
| 1-3% | BRAF | Exon15 | Kinase | V600E1 | c.1799T > A |
| | EGFR | Exon18 | Kinase | G719A | c.2156G > C |
| | | Exon19 | | E746_A750del | c.2235_2249del15 |
| | | | | E746_A750del | c.2236_2250del15 |
| | | Exon20 | | T790M | c.2369C > T |
| | | | | C797S T > A | c.2389T > A |
| | | | | C797S G > C | c.2390G > C |
| | | | | S768I | c.2303G > T |
| | | | | V769_D770insASV | c.2307_2308ins9GCCAGCGTG |
| | | | | H773_V774insH | c.2319_2320insCAC |
| | | | | D770_N771insG | c.2310_2311insGGT |
| | | | | D770_N771insSVD | 2311_2312ins9GCGTGGACA |
| | | | | p.V769_D770insASV | 2309_2310AC > CCAGCGTGGAT |
| | | Exon21 | | p.L858R (T > G) | c.2573T > G |
| 1% | AKT1 | Exon4 | Pleckstrin | E17K | c.49G > A |
| | | | homology | | |
| 1% | MEK1 | Exon2 | Alpha helix | K57N | c.171G > T |
| 2~4% | HER2 | Exon20 | Kinase | p.Ala775_Gly776insYVMA | c.2324_2325ins12 |

| | DNA Mutation | | | | | |
|---|---|---|---|---|---|---|
| | | Frequency in | | | | Total |
| | | each Mutated | Mutated sample | | | Mutation |
| | COSMIC | Gene in My | in Lung sample | % in | | in each |
| Frequency | Number | Cancer Genome | from COSMIC | each | Total % | gene |
| 15-25% | 518 | 17% | 1277 | 19.76% | 78.87% | 6461 |
| | 516 | 20% | 1478 | 22.88% | | |
| | 521 | 42% | 2341 | 36.23% | | |
| 1% | 583 | 25~50% | 26 | 29.21% | 29.21% | 89 |
| 1-3% | 760 | 8.9% | 26 | 9.67% | 43.87% | 269 |
| | 763 | 26.7% | 69 | 25.65% | | |
| | 775 | 12.9% | 23 | 8.55% | | |
| 1-3% | 476 | 55% | 127 | 43.34% | 43.34% | 293 |
| | 6239 | 0.60% | 56 | 0.73% | 0.73% | 7700 |
| | 6223 | 48% | 1034 | 13.43% | 19.70% | |
| | 6225 | | 483 | 6.27% | | |
| | 6240 | <5% | 300 | 3.90% | 5.04% | |
| | 6493937 | — | 1 | 0.01% | | |
| | 5945664 | — | 2 | 0.03% | | |
| | 6241 | 1.5~3% | 41 | 0.53% | | |
| | 12376 | 4~9.2% | 10 | 0.13% | | |
| | 12377 | | 6 | 0.08% | | |
| | 12378 | | 8 | 0.10% | | |
| | 13428 | | 19 | 0.25% | | |
| | 13558 | | 1 | 0.01% | | |
| | 6224 | 43% | 2334 | 30.31% | 30.31% | |
| 1% | 33765 | 88% | 16 | 61.54% | 61.54% | 26 |
| 1% | 1235478 | | 7 | 25.00% | 25.00% | 28 |
| 2~4% | 20959 | 83~100% | 47 | 28.14% | 28.14% | 167 |

TABLE A2
RNA mutations.

| | RNA Variant | | | | | | | | | |
|---|---|---|---|---|---|---|---|---|---|---|
| | | | | | Number | | | | | % in |
| | | Fusion | Final | Number in | of total | | | Mutated | Tested | lung |
| Frequency | Gene | Name | Name | lung cancer | variant | % | Coverage | sample | sample | cancer |
| 3-7% | ALK | V1 | E13; A20 | 175 | 387 | 45.22% | 91.73% | 355 | 9765 | 3.64% |
| | | V2 | E20; A20 | 51 | | 13.18% | | | | |
| | | V3a | E6a; A20 | 98 | | 25.32% | | | | |
| | | V3b | E6b; A20 | 31 | | 8.01% | | | | |
| 1~2% | ROS | CD74-ROS1 | C6; R32 | 3 | 36 | 8.3% | 100.00% | 36 | 1678 | 2.15% |
| | | | C6; R34 | 33 | | 91.7% | | | | |
| | | SLC34A2-ROS1 | S4; R32 | 6 | 9 | 66.7% | 77.8% | 7 | 1322 | 0.53% |
| | | | S4; R34 | 1 | | 11.1% | | | | |
| 1~2% | RET | KIF5B-RET | K15: R11 | 1 | 59 | 1.7% | 91.5% | 54 | 4009 | 1.35% |
| | | | K15; R12 | 36 | | 61.0% | | | | |
| | | | K16; R12 | 12 | | 20.3% | | | | |
| | | | K22; R12 | 3 | | 5.1% | | | | |
| | | | K23; R12 | 2 | | 3.4% | | | | |
| 1~5% | NTRK1 | CD74-NTRK1 | C8; N12 | 1 | 1 | 100.0% | 100.0% | No Record in | | — |
| | | | | | | | | COSMIC | | |
| 4% | cMET | cMET Exon14 skipping | — | | | | | | | 4% |
| | | | | | | | | | Total | 12% |

What is claimed is:

1. A method for detecting the presence of mutations in the KRAS, NRAS, PIK3CA, BRAF, EGFR, AKT1, MEK1, and HER2 genes, the method comprising:
(a) isolating DNA from a sample;
(b) amplifying the isolated DNA by polymerase chain reaction (PCR) using primer pairs specific for the loci of one or more DNA mutations in each of the KRAS, NRAS, PIK3CA, BRAF, EGFR, AKT1, MEK1, and HER2 genes;
(c) hybridizing the amplified DNA with at least seven probes, said at least seven probes comprising one or more probes specific for a DNA mutation in each of the KRAS, NRAS, PIK3CA, BRAF, EGFR, AKT1, MEK1, and HER2 genes, wherein each of said at least seven probes is coupled to a microcarrier, and wherein each of the microcarriers comprises an identifier corresponding to the probe coupled thereto;
(d) detecting presence or absence of hybridization of the amplified DNA with said at least seven probes, wherein hybridization between the amplified DNA and one of the probes indicates the presence of the DNA mutation corresponding to the probe;
(e) detecting the identifiers of the microcarriers; and
(f) correlating the detected identifiers of the microcarriers with the detected presence or absence of hybridization of the amplified DNA to the corresponding probes of the microcarriers.

2. The method of claim 1, wherein the KRAS, NRAS, PIK3CA, BRAF, EGFR, AKT1, MEK1, and HER2 genes are human genes.

3. The method of claim 1 or claim 2, wherein step (b) comprises amplifying the isolated DNA by PCR in the presence of at least seven blocking nucleic acids, wherein each of said at least seven blocking nucleic acids hybridizes with a wild-type DNA locus corresponding with one of the DNA mutations in the KRAS, PIK3CA, BRAF, EGFR, AKT1, MEK1, or HER2 genes and prevents amplification of the wild-type DNA locus.

4. The method of claim 3, wherein each of said at least seven blocking nucleic acids comprises:
a single-stranded oligonucleotide that hybridizes with the corresponding wild-type DNA locus; and
a 3′ terminal moiety that blocks extension from the single-stranded oligonucleotide.

5. The method of claim 4, wherein the 3′ terminal moiety comprises one or more inverted deoxythymidines.

6. The method of any one of claims 3-5, wherein each of said at least seven blocking nucleic acids comprises one or more modified nucleotides selected from the group consisting of locked nucleic acids (LNAs), peptide nucleic acids (PNAs), hexose nucleic acids (HNAs), threose nucleic acids (TNAs), glycol nucleic acids (GNAs), and cyclohexenyl nucleic acids (CeNAs).

7. The method of any one of claims 2-6, wherein the one or more DNA mutations in the KRAS gene comprise one or more DNA mutations encoding a G12D, G12V, or G12C mutated KRAS protein.

8. The method of claim 7, wherein the one or more DNA mutations in the KRAS gene comprise DNA mutations encoding G12D, G12V, and G12C mutated KRAS proteins.

9. The method of claim 8, wherein the probes specific for one or more DNA mutations in the KRAS gene comprise:
(1) a first probe comprising a sequence selected from the group consisting of TAGTTGGAGCT (SEQ ID NO:38), TGTGGTAGTTG (SEQ ID NO:40), TGATGGCGTAG (SEQ ID NO:42), TGGAGCTGATGGC (SEQ ID NO:44), and GCGTAGGCAAG (SEQ ID NO:46);
(2) a second probe comprising a sequence selected from the group consisting of CTGTTGGCGTAGG (SEQ ID NO:48), GTAGTTGGAGCTG (SEQ ID NO:50), TGGAGCTGTTGGC (SEQ ID NO:52), TTGTGGTAGTTGG (SEQ ID NO:54), and GGCGTAGGCAAGA (SEQ ID NO:56); and
(3) a third probe comprising a sequence selected from the group consisting of TAGTTGGAGCTT (SEQ ID NO:58), GCGTAGGCAAGA (SEQ ID NO:60), GGAGCTTGTGGC (SEQ ID NO:62), TTGTGGCGTAGG (SEQ ID NO:64), and TGTGGTAGTTGG (SEQ ID NO:66);
wherein each of the three probes is coupled to a microcarrier with a different identifier.

10. The method of claim 9, wherein each of the three probes further comprises eight nucleotides at the 5′ end, and wherein the eight nucleotides at the 5′ end are adenine or thymine nucleotides.

11. The method of claim 10, wherein the probes specific for one or more DNA mutations in the KRAS gene comprise:
(1) a first probe comprising a sequence selected from the group consisting of TTTTTTTTTTTTAATAGTTGGAGCT (SEQ ID NO:39), TTTTTTTTTTTTAATGTGGTAGTTG (SEQ ID NO:41), TTTTTTTTTTTTAATGATGGCGTAG (SEQ ID NO:43), TTTTTTTTTTTATGGAGCTGATGGC (SEQ ID NO:45), and TTTTTTTTTTTTAAGCGTAGGCAAG (SEQ ID NO:47);
(2) a second probe comprising a sequence selected from the group consisting of TTTTTTTTTTTACTGTTGGCGTAGG (SEQ ID NO:49), TTTTTTTTTTTAGTAGTTGGAGCTG (SEQ ID NO:51), TTTTTTTTTTATGGAGCTGTTGGC (SEQ ID NO:53), TTTTTTTTTTTATTGTGGTAGTTGG (SEQ ID NO:55), and TTTTTTTTTTTAGGCGTAGGCAAGA (SEQ ID NO:57); and
(3) a third probe comprising a sequence selected from the group consisting of TTTTTTTTTTTAATAGTTGGAGCTT (SEQ ID NO:59), TTTTTTTTTTTAAGCGTAGGCAAGA (SEQ ID NO:61), TTTTTTTTTTTAAGGAGCTTGTGGC (SEQ ID NO:63), TTTTTTTTTTTAATTGTGGCGTAGG (SEQ ID NO: 65), and TTTTTTTTTTTAATGTGGTAGTTGG (SEQ ID NO:67);
wherein each of the three probes is coupled to a microcarrier with a different identifier.

12. The method of any one of claims 7-11, wherein step (b) comprises amplifying the isolated DNA by PCR using a primer pair comprising the sequences GTACTGGTGGAGTATTTGATAGTG (SEQ ID NO:1) and CGTCAAGGCACTCTTGCCTAC (SEQ ID NO:2).

13. The method of any one of claims 7-12, wherein step (b) comprises amplifying the isolated DNA by PCR in the presence of a blocking nucleic acid that hybridizes with a wild-type KRAS DNA locus corresponding with one of the KRAS DNA mutations and prevents amplification of the wild-type KRAS DNA locus, and wherein the blocking nucleic acid comprises the sequence TACGCCACCAGCT(invdT)ₙ, wherein n is 1, 2, or 3 (SEQ ID NO:281); TTGGAGCTGGTGGCGTA(invdT)ₙ, wherein n is 1, 2, or 3 (SEQ ID NO:282); GCTGGTGGCGTAGGCA(invdT)ₙ, wherein n is 1, 2, or 3 (SEQ ID NO:283); GCTGGTGGCGTAGGC(invdT)ₙ, wherein n is 1, 2, or 3 (SEQ ID NO:284); or TTGGAGCTGGTGGCGT(invdT)ₙ, wherein n is 1, 2, or 3 (SEQ ID NO:285); with italicized nucleic acids representing locked nucleic acids.

14. The method of any one of claims 2-13, wherein the one or more DNA mutations in the PIK3CA gene comprise one or more DNA mutations encoding an E542K or E545K mutated PIK3CA protein.

15. The method of claim 14, wherein the one or more DNA mutations in the PIK3CA gene comprise DNA mutations encoding E542K and E545K mutated PIK3CA proteins.

16. The method of claim 15, wherein the probes specific for one or more DNA mutations in the PIK3CA gene comprise:
(1) a first probe comprising a sequence selected from the group consisting of GCTCAGTGATTTTAG (SEQ ID NO:87), TGCTCAGTGATTTT (SEQ ID NO: 89), GCTCAGTGATTTTAG (SEQ ID NO:91), CCTGCTCAGTGATTTTA (SEQ ID NO:93), and CTCAGTGATTTTAGA (SEQ ID NO:95); and
(2) a second probe comprising a sequence selected from the group consisting of TTCTCCTGCTTA (SEQ ID NO:97), CTCCTGCTTAGT (SEQ ID NO:99), TCTCCTGCTTAG (SEQ ID NO:101), TCCTGCTTAGTG (SEQ ID NO:103), and CTCCTGCTTAGTGA (SEQ ID NO:105);
wherein each of the two probes is coupled to a microcarrier with a different identifier.

17. The method of claim 16, wherein each of the two probes further comprises eight nucleotides at the 5′ end, and wherein the eight nucleotides at the 5′ end are adenine or thymine nucleotides.

18. The method of claim 17, wherein the probes specific for one or more DNA mutations in the PIK3CA gene comprise:
(1) a first probe comprising a sequence selected from the group consisting of TTTTTTTTTAGCTCAGTGATTTTAG (SEQ ID NO:88), TTTTTTTTTTGCTCAGTGATTTT (SEQ ID NO:90), TTTTTTTTTAGCTCAGTGATTTTAG (SEQ ID NO:92), TTTTTTTCCTGCTCAGTGATTTTA (SEQ ID NO: 94), and TTTTTTTTTTTCTCAGTGATTTTAGA (SEQ ID NO: 96); and
(2) a second probe comprising a sequence selected from the group consisting of TTTTTTTTTTTTTCTCCTGCTTA (SEQ ID NO:98), TTTTTTTTTTTTTCTCCTGCTTAGT (SEQ ID NO:100), TTTTTTTTTTTATCTCCTGCTTAG (SEQ ID NO:102), TTTTTTTTTTTTTTTCCTGCTTAGTG (SEQ ID NO:104), and TTTTTTTTTTTTTCTCCTGCTTAGTGA (SEQ ID NO:106);
wherein each of the three probes is coupled to a microcarrier with a different identifier.

19. The method of any one of claims 14-18, wherein step (b) comprises amplifying the isolated DNA by PCR using a primer pair comprising the sequences CAATTTCTACAAGAGATCCTCTCTCT (SEQ ID NO:5) and CTCCATTTTAGCACTTACCTGTGAC (SEQ ID NO:6).

20. The method of any one of claims 14-19, wherein step (b) comprises amplifying the isolated DNA by PCR in the presence of a blocking nucleic acid that hybridizes with a wild-type PIK3CA DNA locus corresponding with one of the PIK3CA DNA mutations and prevents amplification of the wild-type PIK3CA DNA locus, and wherein at least one of the at least seven blocking nucleic acids comprises the sequence CTGAAATCACTGAGCAGG(invdT)ₙ, wherein n is 1, 2, or 3 (SEQ ID NO:291); TCTCTGAAATCACTGAGCAGG(invdT)ₙ, wherein n is 1, 2, or 3 (SEQ ID NO:292); TCTCTGAAATCACTGAGCAGG(invdT)ₙ, wherein n is 1, 2, or 3 (SEQ ID NO:293); TCTCTGAAATCACTGAGCAGG(invdT)ₙ, wherein n is 1, 2, or 3 (SEQ ID NO:294); or TCTCTGAATTCACTGAGCAGG(invdT)ₙ, wherein n is 1, 2, or 3 (SEQ ID NO:295); with italicized nucleic acids representing locked nucleic acids.

21. The method of any one of claims 2-20, wherein the one or more DNA mutations in the PIK3CA gene comprise a DNA mutation encoding an H1047R mutated PIK3CA protein.

22. The method of claim 21, wherein the probes specific for one or more DNA mutations in the PIK3CA gene comprise:
(1) a first probe comprising a sequence selected from the group consisting of GATGCACGTCATG (SEQ ID NO:107), TGAATGATGCACG (SEQ ID NO:109), TGATGCACGTC (SEQ ID NO:111), AATGATGCACGTCA (SEQ ID NO:113), and AATGATGCACGTC (SEQ ID NO:115);
wherein the first probe is coupled to a microcarrier with an identifier.

23. The method of claim 22, wherein the first probe further comprises eight nucleotides at the 5′ end, and wherein the eight nucleotides at the 5′ end are adenine or thymine nucleotides.

24. The method of claim 23, wherein the probes specific for one or more DNA mutations in the PIK3CA gene comprise:
(1) a first probe comprising a sequence selected from the group consisting of TTTTTTTTTTTTTTTGATGCACGTCATG (SEQ ID NO:108), TTTTTTTTTTTGAATGATGCACG (SEQ ID NO:110), TTTTTTTTTTTTTGATGCACGTC (SEQ ID NO:112), TTTTTTTTTTTTAATGATGCACGTCA (SEQ ID NO:114), and TTTTTTTTTTTTAATGATGCACGTC (SEQ ID NO:116);
wherein the first probe is coupled to a microcarrier with an identifier.

25. The method of any one of claims 21-24, wherein step (b) comprises amplifying the isolated DNA by PCR using a primer pair comprising the sequences ACCCTAGCCTTAGATAAAACTGAGC (SEQ ID NO:7) and TTTGTTGTCCAGCCACCATGA (SEQ ID NO: 8).

26. The method of any one of claims 21-25, wherein step (b) comprises amplifying the isolated DNA by PCR in the presence a blocking nucleic acid that hybridizes with a wild-type PIK3CA DNA locus corresponding with one of the PIK3CA DNA mutations and prevents amplification of the wild-type PIK3CA DNA locus, and wherein at least one of the at least seven blocking nucleic acids comprises the sequence CACCATGATGTGCAT(invdT)ₙ, wherein n is 1, 2, or 3 (SEQ ID NO:296); CCACCATGATGTGCAT(invdT)ₙ, wherein n is 1, 2, or 3 (SEQ ID NO:297); CACCATGATGTGCAT(invdT)ₙ, wherein n is 1, 2, or 3 (SEQ ID NO:298); CCACCATGATGTGCATCA(invdT)ₙ, wherein n is 1, 2, or 3 (SEQ ID NO:299); or CATGATGTGCA(invdT)ₙ, wherein n is 1, 2, or 3 (SEQ ID NO:300); with italicized nucleic acids representing locked nucleic acids.

27. The method of any one of claims 2-26, wherein the one or more DNA mutations in the BRAF gene comprise one or more DNA mutations encoding a V600E mutated BRAF protein.

28. The method of claim 27, wherein the probe specific for one or more DNA mutations in the BRAF gene comprises a sequence selected from the group consisting of TTTGGTCTAGCTACAGA (SEQ ID NO:79), CTACAGAGAAATCTCGA (SEQ ID NO:81), GTGATTTTGGTCTAGCT (SEQ ID NO:83), and TCTAGCTACAGAGAAAT (SEQ ID NO:85).

29. The method of claim 28, wherein the probe specific for one or more DNA mutations in the BRAF gene further comprises eight nucleotides at the 5′ end, and wherein the eight nucleotides at the 5′ end are adenine or thymine nucleotides.

30. The method of claim 29, wherein the probe specific for one or more DNA mutations in the BRAF gene comprises a sequence selected from the group consisting of TTTFTTAATTGAGAAATCTCGATGGAG (SEQ ID NO:78), TTTFTTAATTTTTGGTCTAGCTACAGA (SEQ ID NO:80), TTTTTTAATTCTACAGAGAAATCTCGA (SEQ ID NO:82), TTTTTTAATTGTGATTTTGGTCTAGCT (SEQ ID NO:84), and TTTTTTAATTTCTAGCTACAGAGAAAT (SEQ ID NO:86).

31. The method of any one of claims 27-30, wherein step (b) comprises amplifying the isolated DNA by PCR using a primer pair comprising the sequences ATAGCCTCAATTCTTACCATCCACAAAATG (SEQ ID NO:9) and CAGATATATTTCTTCATGAAGACCTCACAGTAA (SEQ ID NO:10).

32. The method of any one of claims 27-31, wherein step (b) comprises amplifying the isolated DNA by PCR in the presence of a blocking nucleic acid that hybridizes with a wild-type BRAF DNA locus corresponding with the BRAF DNA mutation and prevents amplification of the wild-type BRAF DNA locus, and wherein at least one of the at least seven blocking nucleic acids comprises the sequence GAGATTTCACTGTAGC(invdT)ₙ, wherein n is 1, 2, or 3 (SEQ ID NO:301); GAGATTTCACTGTAGC(invdT)ₙ, wherein n is 1, 2, or 3 (SEQ ID NO:302); GAGATTTCACTGTAGC(invdT)ₙ, wherein n is 1, 2, or 3 (SEQ ID NO:303); GAGATTCACTGTAGC(invdT)ₙ, wherein n is 1, 2, or 3 (SEQ ID NO:304); or GAGATTTCACTGTAGC(invdT)ₙ, wherein n is 1, 2, or 3 (SEQ ID NO:305); with italicized nucleic acids representing locked nucleic acids.

33. The method of any one of claims 2-32, wherein the one or more DNA mutations in the EGFR gene comprise one or more DNA mutations encoding a G719A mutated EGFR protein.

34. The method of claim 33, wherein the probe specific for one or more DNA mutations in the EGFR gene comprises a sequence selected from the group consisting of TCAAAGTGCTGGCCTC (SEQ ID NO:117), AGATCAAAGTGCTGGCCTCCG (SEQ ID NO:119), AAAGTGCTGGCCT (SEQ ID NO:121), AGTGCTGGCCT (SEQ ID NO:123), and AAGTGCTGGCCTC (SEQ ID NO:125).

35. The method of claim 34, wherein the probe specific for one or more DNA mutations in the EGFR gene further comprises eight nucleotides at the 5′ end, and wherein the eight nucleotides at the 5′ end are adenine or thymine nucleotides.

36. The method of claim 35, wherein the probe specific for one or more DNA mutations in the EGFR gene comprises a sequence selected from the group consisting of TTTTTTTTTTCAAAGTGCTGGCCTC (SEQ ID NO:118), TTTTTTAGATCAAAGTGCTGGCCTCCG (SEQ ID NO:120), TTTTTTTTTTTAAAGTGCTGGCCT (SEQ ID NO: 122), TTTTTTTTTTTTTAGTGCTGGCCT (SEQ ID NO: 124), and TTTTTTTTTTTTAAGTGCTGGCCTC (SEQ ID NO: 126).

37. The method of any one of claims 33-36, wherein step (b) comprises amplifying the isolated DNA by PCR using a primer pair comprising the sequences CTTGTGGAGCCTCTTACACCC (SEQ ID NO:11) and TGCCGAACGCACCGGA (SEQ ID NO:12).

38. The method of any one of claims 33-37, wherein step (b) comprises amplifying the isolated DNA by PCR in the presence of a blocking nucleic acid that hybridizes with a wild-type EGFR DNA locus corresponding with the EGFR DNA mutation and prevents amplification of the wild-type EGFR DNA locus, and wherein at least one of the at least seven blocking nucleic acids comprises the sequenceCGGAGCCCAGCACTTTGA (invdT)ₙ, wherein n is 1, 2, or 3 (SEQ ID NO:306); CGCACCGGAGCCCAGCACT (invdT)ₙ, wherein n is 1, 2, or 3 (SEQ ID NO:307); GAGCCCAGCAC (invdT)ₙ, wherein n is 1, 2, or 3 (SEQ ID NO:308); CGCACCGGAGCCCAGCAC (invdT)ₙ, wherein n is 1, 2, or 3 (SEQ ID NO:309); or CGCACCGGAGCCCAGCACTTA (invdT)ₙ, wherein n is 1, 2, or 3 (SEQ ID NO:310); with italicized nucleic acids representing locked nucleic acids.

39. The method of any one of claims 2-38, wherein the one or more DNA mutations in the EGFR gene comprise one or more DNA mutations encoding an E746_A750del mutated EGFR protein.

40. The method of claim 39, wherein the probes specific for one or more DNA mutations in the EGFR gene comprise:
(1) a first probe comprising a sequence selected from the group consisting of AATCAAAACATCTCCGAAAG (SEQ ID NO:128), CAAAACATCTCCG (SEQ ID NO:130), AACATCTCCG (SEQ ID NO:132), and AAACATCTCCGAAAGCC (SEQ ID NO:134); and
(2) a second probe comprising a sequence selected from the group consisting of AATCAAGACATCTCCGA (SEQ ID NO:136), GCAATCAAGACATCTCCGA (SEQ ID NO:138), AATCAAGACATCTC (SEQ ID NO:140), AATCAAGACATCTCCGAAAGC (SEQ ID NO:142), and CAAGACATCTCCGA (SEQ ID NO:144);
wherein each of the two probes is coupled to a microcarrier with a different identifier.

41. The method of claim 40, wherein each of the two probes further comprises eight nucleotides at the 5′ end, and wherein the eight nucleotides at the 5′ end are adenine or thymine nucleotides.

42. The method of claim 41, wherein the probes specific for one or more DNA mutations in the EGFR gene comprise:
(1) a first probe comprising a sequence selected from the group consisting of TTTTTTTTTAATCAAAACATCTCCG (SEQ ID NO:127), TTTTTTTTTAATCAAAACATCTCCGAAAG (SEQ ID NO:129), TTTTTTTTTTTACAAAACATCTCCG (SEQ ID NO:131), TTTTTTTTTTTTTTTAACATCTCCG (SEQ ID NO:133), and TTTTTTTTTTTTTTAAACATCTCCGAAAGCC (SEQ ID NO:135); and
(2) a second probe comprising a sequence selected from the group consisting of TTTTTTTTAATCAAGACATCTCCGA (SEQ ID NO:137), TTTTTTGCAATCAAGACATCTCCGA (SEQ ID NO:139), TTTTTTTTAATCAAGACATCTC (SEQ ID NO:141), TTTTTTTTAATCAAGACATCTCCGAAAGC (SEQ ID NO:143), and TTTTTTTTTTTCAAGACATCTCCGA (SEQ ID NO:145);
wherein each of the two probes is coupled to a microcarrier with a different identifier.

43. The method of any one of claims 39-42, wherein step (b) comprises amplifying the isolated DNA by PCR using a primer pair comprising the sequences GCCAGTTAACGTCTTCCTTCTC (SEQ ID NO:13) and ATCGAGGATTTCCTTGTTGGCTT (SEQ ID NO:14).

44. The method of any one of claims 39-43, wherein step (b) comprises amplifying the isolated DNA by PCR in the presence of a blocking nucleic acid that hybridizes with a wild-type EGFR DNA locus corresponding with the EGFR DNA mutation and prevents amplification of the wild-type EGFR DNA locus, and wherein at least one of the at least seven blocking nucleic acids comprises the sequence CGGAGATGTTGCTTCTCTTAATTCC(invdT)ₙ, wherein n is 1, 2, or 3 (SEQ ID NO:311); CGGAGATGTTGCTTCTCT(invdT)ₙ, wherein n is 1, 2, or 3 (SEQ ID NO:312); GTTGCTTCTCTTAATTCC(invdT)ₙ, wherein n is 1, 2, or 3 (SEQ ID NO:313); ATGTTGCTTCTCT(invdT)ₙ, wherein n is 1, 2, or 3 (SEQ ID NO:314); or TTGCTTCTCTTA(invdT)ₙ, wherein n is 1, 2, or 3 (SEQ ID NO:315); with italicized nucleic acids representing locked nucleic acids.

45. The method of any one of claims 2-44, wherein the one or more DNA mutations in the EGFR gene comprise one or more DNA mutations encoding a T790M, C797S, S768I, V769_D770insASV, H773_V774insH, D770_N771insG, or D770_N771insSVD mutated EGFR protein.

46. The method of claim 45, wherein the one or more DNA mutations in the EGFR gene comprise DNA mutations encoding T790M, C797S, S768I, V769_D770insASV, H773_V774insH, D770_N771insG, and D770_N771insSVD mutated EGFR proteins.

47. The method of claim 46, wherein the probes specific for one or more DNA mutations in the EGFR gene comprise:
(1) a first probe comprising a sequence selected from the group consisting of GAGATGCATGATGA (SEQ ID NO:146), TGAGATGCATGATGAG (SEQ ID NO:147), ATGAGATGCATGATGAG (SEQ ID NO:148), TGAGCTGCATGATGA (SEQ ID NO:149), and CATGAGATGCATGATGA (SEQ ID NO:150);
(2) a second probe comprising a sequence selected from the group consisting of CCAGGAGGCTGCCG (SEQ ID NO:461), CAGGAGGCTGCCGA (SEQ ID NO:463), TCCAGGAGGCTGCC (SEQ ID NO:465), CCAGGAGGCTGCC (SEQ ID NO:467), and CAGGAGGCTGCC (SEQ ID NO:469);
(3) a third probe comprising a sequence selected from the group consisting of CCAGGAGGGAGCC (SEQ ID NO:471), CCAGGAGGGAGCCG (SEQ ID NO:473), TCCAGGAGGGAGCC (SEQ ID NO:475), CAGGAGGGAGCCG (SEQ ID NO:477), and CAGGAGGGAGCCGA (SEQ ID NO:479);
(4) a fourth probe comprising a sequence selected from the group consisting of ATGGCCATCTTGG (SEQ ID NO:421), GGCCATCTTGGA (SEQ ID NO:423), GATGGCCATCTTG (SEQ ID NO:425), TGATGGCCATCTTG (SEQ ID NO:427), and TGGCCATCTTGG (SEQ ID NO:429);
(5) a fifth probe comprising a sequence selected from the group consisting of GTGATGGCCGG (SEQ ID NO:431), TGATGGCCGGCG (SEQ ID NO:433), GTGATGGCCGGCGT (SEQ ID NO:435), GATGGCCGGCGT (SEQ ID NO:437), and GATGGCCCGCGTG (SEQ ID NO:439);
(6) a sixth probe comprising a sequence selected from the group consisting of AACCCCCATCACGT (SEQ ID NO:441), GACAACCCCCATCACG (SEQ ID NO:443), CGTGGACAACCCCCATCA (SEQ ID NO:445), CCCATCACGTGT (SEQ ID NO:447), and TGGACAACCCCCATCAC (SEQ ID NO:449); and
(7) a seventh probe comprising a sequence selected from the group consisting of GCCAGCGTGGACGG (SEQ ID NO:451), CGTGGACGGTAACC (SEQ ID NO:453), GACGGTAACCCCC (SEQ ID NO:455), CCAGCGTGGACGGT (SEQ ID NO:457), and GCCAGCGTGGACGGTA (SEQ ID NO:459);
wherein each of the seven probes is coupled to a microcarrier with a different identifier.

48. The method of claim 47, wherein each of the seven probes specific for one or more DNA mutations in the EGFR gene further comprises eight nucleotides at the 5′ end, and wherein the eight nucleotides at the 5′ end are adenine or thymine nucleotides.

49. The method of claim 48, wherein the probes specific for one or more DNA mutations in the EGFR gene comprise:
(1) a first probe comprising a sequence selected from the group consisting of TTTTTTTTTTTGAGATGCATGATGA (SEQ ID NO:352), TTTTTTTTTTGAGATGCATGATGAG (SEQ ID NO:353), TTTTTTTTATGAGATGCATGATGAG (SEQ ID NO:354), TTTTTTTTTTTGAGCTGCATGATGA (SEQ ID NO:355), and TTTTTTTTCATGAGATGCATGATGA (SEQ ID NO:356);
(2) a second probe comprising a sequence selected from the group consisting of TTTTTTTTTTTACCAGGAGGCTGCCG (SEQ ID NO:462), TTTTTTTTTTTACAGGAGGCTGCCGA (SEQ ID NO:464), TTTTTTTTTTTATCCAGGAGGCTGCC (SEQ ID NO:466), TTTTTTTTTTTACCAGGAGGCTGCC (SEQ ID NO:468), and TTTTTTTTTTTACAGGAGGCTGCC (SEQ ID NO:470),
(3) a third probe comprising a sequence selected from the group consisting of TTTTTTTTTTTACCAGGAGGGAGCC (SEQ ID NO:472), TTTTTTTTTTTACCAGGAGGGAGCCG (SEQ ID NO:474), TTTTTTTTTTTATCCAGGAGGGAGCC (SEQ ID NO:476), TTTTTTTTTTTACAGGAGGGAGCCG (SEQ ID NO:478), and TTTTTTTTTTTACAGGAGGGAGCCGA (SEQ ID NO:480);
(4) a fourth probe comprising a sequence selected from the group consisting of TTTTTTTTTATGGCCATCTTGG (SEQ ID NO:422), TTTTTTTTTTAGGCCATCTTGGA (SEQ ID NO:424), TTTTTTTAGATGGCCATCTTG (SEQ ID NO:426), TTTTTTTTGATGGCCATCTTG (SEQ ID NO:428), and TTTTTTTTTTTGGCCATCTTGG (SEQ ID NO:430);
(5) a fifth probe comprising a sequence selected from the group consisting of TTTTTTTTTTTGTGATGGCCGG (SEQ ID NO:432), TTTTTTTTTTTTTGATGGCCGGCG (SEQ ID NO:434), TTTTTTTTTTTGTGATGGCCGGCGT (SEQ ID NO:436), TTTTTTTTTTTTTGATGGCCGGCGT (SEQ ID NO:438), and TTTTTTTTTTTTTGATGGCCCGCGTG (SEQ ID NO:440);
(6) a sixth probe comprising a sequence selected from the group consisting of TTTTTTTTTTTAACCCCCATCACGT (SEQ ID NO:442), TTTTTTTTGACAACCCCCATCACG (SEQ ID NO:444), TTTTCGTGGACAACCCCCATCA (SEQ ID NO:446), TTTTTTTTTTTTCCCATCACGTGT (SEQ ID NO:448), and TTTTTTTTGGACAACCCCCATCAC (SEQ ID NO:450); and
(7) a seventh probe comprising a sequence selected from the group consisting of TTTTTTTTTTTGCCAGCGTGGACGG (SEQ ID NO:452), TTTTTTTTTTTCGTGGACGGTAACC (SEQ ID NO:454), TTTTTTTTTTTGACGGTAACCCCC (SEQ ID NO:456), TTTTTTTTTTCCAGCGTGGACGGT (SEQ ID NO:458), and TTTTTTTGCCAGCGTGGACGGTA (SEQ ID NO:460);
wherein each of the seven probes is coupled to a microcarrier with a different identifier.

50. The method of any one of claims 45-49, wherein step (b) comprises amplifying the isolated DNA by PCR using a primer pair comprising the sequences CCTCCACCGTGCAGATCATC (SEQ ID NO:15) and TTCCCTGATTACCTTTGCGAT (SEQ ID NO:16); a primer pair comprising the sequences CCACACTGACGTGCCTCT (SEQ ID NO:511) and GCACACGTAGGGGTTGTCCAAGA (SEQ ID NO:512); a primer pair comprising the sequences CCACACTGACGTGCCTCT (SEQ ID NO: 513) and GTACACGCTGGCCACGCCG (SEQ ID NO:514); a primer pair comprising the sequences CCACACTGACGTGCCTCT (SEQ ID NO:515) and CAGGCGGCACACGTGAT (SEQ ID NO:516); and/or a primer pair comprising the sequences CCACACTGACGTGCCTCT (SEQ ID NO: 517) and AGGCGGCACACGTGCGGGTTAC (SEQ ID NO:518).

51. The method of any one of claims 45-50, wherein step (b) comprises amplifying the isolated DNA by PCR in the presence of a blocking nucleic acid that hybridizes with a wild-type EGFR DNA locus corresponding with the EGFR DNA mutation and prevents amplification of the wild-type EGFR DNA locus, and wherein at least one of the at least seven blocking nucleic acids comprises the sequence CATCACGCAGCTCATG(invdT)ₙ, wherein n is 1, 2, or 3 (SEQ ID NO:316); TGCAGCTCATCACGCAGC(invdT)ₙ, wherein n is 1, 2, or 3 (SEQ ID NO:317); TCATCACGCAGCTCAT(invdT)ₙ, wherein n is 1, 2, or 3 (SEQ ID NO:318); TCATCACGCAGC(invdT)ₙ, wherein n is 1, 2, or 3 (SEQ ID NO: 319); or CTCATCACGCAGC(invdT)ₙ, wherein n is 1, 2, or 3 (SEQ ID NO:320); with italicized nucleic acids representing locked nucleic acids.

52. The method of any one of claims 2-51, wherein the one or more DNA mutations in the EGFR gene comprise one or more DNA mutations encoding an L858R mutated EGFR protein.

53. The method of claim 52, wherein the probes specific for one or more DNA mutations in the EGFR gene comprise: a first probe comprising a sequence selected from the group consisting of ATTTTGGGCGGGCC (SEQ ID NO:151), TTGGGCGGGCCAAA (SEQ ID NO:153), GCGGGCCAAACT (SEQ ID NO:155), GGGCGGGCCAAACT (SEQ ID NO:157), and TGGGCGGGCCA (SEQ ID NO:159);
wherein the first probe is coupled to a microcarrier with an identifier.

54. The method of claim 53, wherein the first probe further comprises eight nucleotides at the 5′ end, and wherein the eight nucleotides at the 5′ end are adenine or thymine nucleotides.

55. The method of claim 54, wherein the probes specific for one or more DNA mutations in the EGFR gene comprise: a first probe comprising a sequence selected from the group consisting of TTTTTTTATTTTGGGCGGGCC (SEQ ID NO:152), TTTTTTTTAATTGGGCGGGCCAAA (SEQ ID NO:154), TTTTTTTAAAAAAGCGGGCCAAACT (SEQ ID NO:156), TTTTTTTTAAAAGGGCGGGCCAAACT (SEQ ID NO:158), and TTTTTTTTAAATGGGCGGGCCA (SEQ ID NO:160);
wherein the first probe is coupled to a microcarrier with an identifier.

56. The method of any one of claims 52-55, wherein step (b) comprises amplifying the isolated DNA by PCR using a primer pair comprising the sequences GGAGGACCGTCGCTTGG (SEQ ID NO: 17) and TCTTTCTCTTCCGCACCCAG (SEQ ID NO:18).

57. The method of any one of claims 52-56, wherein step (b) comprises amplifying the isolated DNA by PCR in the presence of a blocking nucleic acid that hybridizes with a wild-type EGFR DNA locus corresponding with the EGFR DNA mutation and prevents amplification of the wild-type EGFR DNA locus, and wherein at least one of the at least seven blocking nucleic acids comprises the sequence CCAGCAGTTTGGCCAGCCCT(invdT)ₙ, wherein n is 1, 2, or 3 (SEQ ID NO:321); CCAGCAGTTTGGCCAGCCCT(invdT)ₙ, wherein n is 1, 2, or 3 (SEQ ID NO:322); CCAGCAGTTTGGCCAGCCCT(invdT)ₙ, wherein n is 1, 2, or 3 (SEQ ID NO:323); AGCAGTTTGGCCAGCC(invdT)ₙ, wherein n is 1, 2, or 3 (SEQ ID NO:324); or CCAGCAGTTTGGCCAGCCCT(invdT)ₙ, wherein n is 1, 2, or 3 (SEQ ID NO:325); with italicized nucleic acids representing locked nucleic acids.

58. The method of any one of claims 2-57, wherein the one or more DNA mutations in the AKT1 gene comprise one or more DNA mutations encoding an E17K mutated AKT1 protein.

59. The method of claim 58, wherein the probe specific for one or more DNA mutations in the AKT1 gene comprises a sequence selected from the group consisting of TGTAGGGAAGTACA (SEQ ID NO:370), TCTGTAGGGAAGTAC (SEQ ID NO:372), GTCTGTAGGGAAGTACAT (SEQ ID NO:374), CCGCACGTCTGTAGGGA (SEQ ID NO:376), and ACGTCTGTAGGGAAGTA (SEQ ID NO:378).

60. The method of claim 59, wherein the probe specific for one or more DNA mutations in the AKT1 gene further comprises seven nucleotides at the 5′ end, and wherein the seven nucleotides at the 5′ end are adenine or thymine nucleotides.

61. The method of claim 60, wherein the probe specific for one or more DNA mutations in the AKT1 gene comprises a sequence selected from the group consisting of TTTTTTTTTTTTTTGTAGGGAAGTACA (SEQ ID NO:371), TTTTTTTTTTTTCTGTAGGGAAGTAC (SEQ ID NO:373), TTTTTTTGTCTGTAGGGAAGTACAT (SEQ ID NO:375), TTTTTTTCCGCACGTCTGTAGGGA (SEQ ID NO:377), and TTTTTTTTACGTCTGTAGGGAAGTA (SEQ ID NO:379).

62. The method of any one of claims 58-61, wherein step (b) comprises amplifying the isolated DNA by PCR using a primer pair comprising the sequences GAGGGTCTGACGGGTAGAGTG (SEQ ID NO:380) and TGGCCGCCAGGTCTTGATGTA (SEQ ID NO:381).

63. The method of any one of claims 58-62, wherein step (b) comprises amplifying the isolated DNA by PCR in the presence of a blocking nucleic acid that hybridizes with a wild-type AKT1 DNA locus corresponding with the AKT1 DNA mutation and prevents amplification of the wild-type AKT1 DNA locus, and wherein at least one of the at least seven blocking nucleic acids comprises the sequence TGTACTCCCCTACA (invdT)ₙ, wherein n is 1, 2, or 3 (SEQ ID NO:382); GATGTACTCCCCT (invdT)ₙ, wherein n is 1, 2, or 3 (SEQ ID NO:383); ATGTACTCCCCTAC (invdT)ₙ, wherein n is 1, 2, or 3 (SEQ ID NO:384); GTACTCCCCTACA (invdT)ₙ, wherein n is 1, 2, or 3 (SEQ ID NO:385); or GATGTACTCCCCTACA (invdT)ₙ, wherein n is 1, 2, or 3 (SEQ ID NO:386); with italicized nucleic acids representing locked nucleic acids.

64. The method of any one of claims 2-63, wherein the one or more DNA mutations in the MEK1 gene comprise one or more DNA mutations encoding a K57N mutated MEK1 protein.

65. The method of claim 64, wherein the probe specific for one or more DNA mutations in the MEK1 gene comprises a sequence selected from the group consisting of TTACCCAGAATCAGAA (SEQ ID NO:387), CCAGAATCAGAAGGTG (SEQ ID NO:389), TTCTTACCCAGAATCA (SEQ ID NO:391), CCTTTCTTACCCAGAATC (SEQ ID NO: 393), and CAGAATCAGAAGGTGG (SEQ ID NO:395).

66. The method of claim 65, wherein the probe specific for one or more DNA mutations in the MEK1 gene further comprises eight nucleotides at the 5′ end, and wherein the eight nucleotides at the 5′ end are adenine or thymine nucleotides.

67. The method of claim 66, wherein the probe specific for one or more DNA mutations in the MEK1 gene comprises a sequence selected from the group consisting of TTTTTAAATTTACCCAGAATCAGAA (SEQ ID NO:388), TTTTTAAATCCAGAATCAGAAGGTG (SEQ ID NO:390), TTTTTAAATTTCTTACCCAGAATCA (SEQ ID NO:392), TTTTTAAATCCTTTCTTACCCAGAATC (SEQ ID NO:394), and TTTTTAAATCAGAATCAGAAGGTGG (SEQ ID NO:396).

68. The method of any one of claims 64-67, wherein step (b) comprises amplifying the isolated DNA by PCR using a primer pair comprising the sequences CTTGATGAGCAGCAGCGAAA (SEQ ID NO:397) and CCTTCAGTTCTCCCACCTTCTG (SEQ ID NO:398).

69. The method of any one of claims 64-68, wherein step (b) comprises amplifying the isolated DNA by PCR in the presence of a blocking nucleic acid that hybridizes with a wild-type MEK1 DNA locus corresponding with the MEK1 DNA mutation and prevents amplification of the wild-type MEK1 DNA locus, and wherein at least one of the at least seven blocking nucleic acids comprises the sequence TCTGCTTCTGGGTAAG (invdT)ₙ, wherein n is 1, 2, or 3 (SEQ ID NO:399); TTCTGCTTCTGGGTAAGA (invdT)ₙ, wherein n is 1, 2, or 3 (SEQ ID NO:400); CACCTTCTGCTTCTGGG (invdT)ₙ, wherein n is 1, 2, or 3 (SEQ ID NO:401); TCTGCTTCTGGGTA (invdT)ₙ, wherein n is 1, 2, or 3 (SEQ ID NO:402); or CACCTTCTGCTTCTGGGTAAGA (invdT)ₙ, wherein n is 1, 2, or 3 (SEQ ID NO:403); with italicized nucleic acids representing locked nucleic acids.

70. The method of any one of claims 2-69, wherein the one or more DNA mutations in the HER2 gene comprise one or more DNA mutations encoding an A775_G776insYVMA mutated HER2 protein.

71. The method of claim 70, wherein the probe specific for one or more DNA mutations in the HER2 gene comprises a sequence selected from the group consisting of ATACGTGATGTCTTAC (SEQ ID NO:404), ACGTGATGGCTTACGT (SEQ ID NO:406), AAGCATACGTGATGGCT (SEQ ID NO:408), GCATACGTGATGGCTT (SEQ ID NO:410), and GCATACGTGATGGCTTA (SEQ ID NO:412).

72. The method of claim 71, wherein the probe specific for one or more DNA mutations in the HER2 gene further comprises five nucleotides at the 5′ end, and wherein the five nucleotides at the 5′ end are adenine or thymine nucleotides.

73. The method of claim 72, wherein the probe specific for one or more DNA mutations in the HER2 gene comprises a sequence selected from the group consisting of TTTTTTTTTATACGTGATGTCTTAC (SEQ ID NO:405), TTTTTTTTTTTACGTGATGGCTTACGT (SEQ ID NO:407), TTTTTAAGCATACGTGATGGCT (SEQ ID NO:409), TTTTTTTGCATACGTGATGGCTT (SEQ ID NO:411), and TTTTTTTGCATACGTGATGGCTTA (SEQ ID NO:413).

74. The method of any one of claims 70-73, wherein step (b) comprises amplifying the isolated DNA by PCR using a primer pair comprising the sequences ATGGCTGTGGTTTGTGATGGT (SEQ ID NO:414) and ACACCAGCCATCACGTAAGACA (SEQ ID NO:415).

75. The method of any one of claims 1-74, wherein the sample is a blood, serum, or plasma sample.

76. The method of claim 75, wherein (a) comprises isolating circulating free DNA (cfDNA) from the sample, and wherein the isolated cfDNA is amplified by PCR in (b).

77. The method of any one of claims 1-76, wherein the method further comprises:
amplifying a positive control DNA sequence using a primer pair specific for the positive control DNA sequence in (b);
hybridizing the amplified positive control gene sequence with a probe specific for the positive control gene sequence in (c), wherein the probe specific for the positive control gene sequence is coupled to a microcarrier with an identifier corresponding to a positive control;
detecting presence or absence of hybridization of the amplified positive control DNA sequence with the probe specific for the positive control gene sequence in (d); and
detecting the identifier corresponding to the positive control in (e).

78. The method of any one of claims 1-77, wherein the method further comprises:
detecting absence of hybridization of the amplified DNA with a microcarrier having an identifier corresponding to a negative control in (d), wherein the microcarrier with the identifier corresponding to the negative control comprises a probe that does not hybridize with the amplified DNA; and
detecting the identifier corresponding to the negative control in (e).

79. A method for detecting the presence of mutations in the ALK, ROS, RET, NTRK1, and cMET genes, the method comprising:
(a) isolating RNA from a sample;
(b) amplifying DNA from the isolated RNA by reverse transcription-polymerase chain reaction (RT-PCR), wherein amplifying the DNA comprises:
(1) generating cDNA specific for each of the ALK, ROS, RET, NTRK1, and cMET genes from the isolated RNA using a first primer specific for each of the ALK, ROS, RET, NTRK1, and cMET genes, the isolated RNA, and a reverse transcriptase, and
(2) amplifying DNA specific for each of the ALK, ROS, RET, NTRK1, and cMET genes by polymerase chain reaction (PCR) using the cDNA generated in (b)(1), a DNA polymerase, the first primer, and a second primer specific for each of the ALK, ROS, RET, NTRK1, and cMET genes that binds to a strand of the cDNA opposite the corresponding first primer and promotes strand extension in a direction opposite that promoted by the corresponding first primer;
(c) hybridizing the amplified DNA with at least five probes, said at least five probes comprising one or more probes specific for a mutation in each of the ALK, ROS, RET, NTRK1, and cMET genes, wherein each of said at least five probes is coupled to a microcarrier, and wherein each of the microcarriers comprises an identifier corresponding to the probe coupled thereto;
(d) detecting presence or absence of hybridization of the amplified DNA with said at least five probes, wherein hybridization between the amplified DNA and one of the probes indicates the presence of the mutation corresponding to the probe;
(e) detecting the identifiers of the microcarriers; and
(f) correlating the detected identifiers of the microcarriers with the detected presence or absence of hybridization of the amplified DNA to the corresponding probes of the microcarriers.

80. The method of claim 79, wherein the ALK, ROS, RET, NTRK1, and cMET genes are human genes.

81. The method of claim 79 or claim 80, wherein one or more of the mutations in the ALK, ROS, RET, and NTRK1 genes comprises a fusion gene.

82. The method of claim 81, wherein each of the mutations in the ALK, ROS, RET, and NTRK1 genes comprises a fusion gene.

83. The method of any one of claims 79-82, wherein the one or more mutations in the ALK gene comprise an EML4-ALK fusion gene.

84. The method of claim 83, wherein the first primer is specific for a region of the EML4 locus, and the second primer is specific for a region of the ALK locus.

85. The method of claim 83, wherein the second primer is specific for a region of the EML4 locus, and the first primer is specific for a region of the ALK locus.

86. The method of any one of claims 83-85, wherein the one or more mutations in the ALK gene comprise one or more of EML E13:ALK E20, EML E20:ALK E20, and EML E6:ALK E20 EML4-ALK fusion genes.

87. The method of claim 86, wherein the one or more mutations in the ALK gene comprise EML E13:ALK E20, EML E20:ALK E20, and EML E6:ALK E20 EML4-ALK fusion genes.

88. The method of claim 87, wherein the probes specific for one or more mutations in the ALK gene comprise:
(1) a first probe comprising a sequence selected from the group consisting of AAAGGACCTAAAGTGT (SEQ ID NO:161), CCTAAAGTGTACCGC (SEQ ID NO:163), GGGAAAGGACCTAAAG (SEQ ID NO:165), AGTGTACCGCCGGAA (SEQ ID NO:167), and TACCGCCGGAAGCACC (SEQ ID NO:169);
(2) a second probe comprising a sequence selected from the group consisting of GACTATGAAATATTGTAC (SEQ ID NO:171), GAAATATTGTACTTGTAC (SEQ ID NO:173), TATTGTACTTGTACCGCC (SEQ ID NO:175), TGTACCGCCGGAAGCAC (SEQ ID NO:177), and CCGCCGGAAGCACCAGGA (SEQ ID NO:179);
(3) a third probe comprising a sequence selected from the group consisting of TGTCATCATCAACCAA (SEQ ID NO:181), ATGTCATCATCAACC (SEQ ID NO:183), GTGTACCGCCGGAAGC (SEQ ID NO:185), TCAACCAAGTGTACCG (SEQ ID NO:187), and TACCGCCGGAAGCACCA (SEQ ID NO:189); and
(4) a fourth probe comprising a sequence selected from the group consisting of CGAAAAAAACAGCCAA (SEQ ID NO:191), TCGCGAAAAAAACAGC (SEQ ID NO:193), GTGTACCGCCGGAAGC (SEQ ID NO:195), TACCGCCGGAAGCACC (SEQ ID NO:197), and ACAGCCAAGTGTACCG (SEQ ID NO:199);
wherein each of the four probes is coupled to a microcarrier with a different identifier.

89. The method of claim 88, wherein each of the four probes further comprises eight nucleotides at the 5′ end, and wherein the eight nucleotides at the 5′ end are adenine or thymine nucleotides.

90. The method of claim 89, wherein the probes specific for one or more mutations in the ALK gene comprise:
(1) a first probe comprising a sequence selected from the group consisting of TTTTTTTTTTAAAGGACCTAAAGTGT (SEQ ID NO:162), TTTTTTTTTTCCTAAAGTGTACCGC (SEQ ID NO: 164), TTTTTTTTTTGGGAAAGGACCTAAAG (SEQ ID NO:166), TTTTTTTTTTAGTGTACCGCCGGAA (SEQ ID NO:168), and TTTTTTTTTTTACCGCCGGAAGCACC (SEQ ID NO: 170);
(2) a second probe comprising a sequence selected from the group consisting of TTTTTTTTTTTTGACTATGAAATATTGTAC (SEQ ID NO:172), TTTTTTTTTTTTGAAATATTGTACTTGTAC (SEQ ID NO:174), TTTTTTTTTTTTTATTGTACTTGTACCGCC (SEQ ID NO:176), TTTTTTTTTTTTTTGTACCGCCGGAAGCAC (SEQ ID NO:178), and TTTTTTTTTTTTCCGCCGGAAGCACCAGGA (SEQ ID NO:180);
(3) a third probe comprising a sequence selected from the group consisting of TTTTTTTTTTTTTTTGTCATCATCAACCAA (SEQ ID NO:182), TTTTTTTTTTTTTTATGTCATCATCAACC (SEQ ID NO:184), TTTTTTTTTTTTTTGTGTACCGCCGGAAGC (SEQ ID NO:186), TTTTTTTTTTTTTTTCAACCAAGTGTACCG (SEQ ID NO:188), and TTTTTTTTTTTTTTTACCGCCGGAAGCACCA (SEQ ID NO:190); and
(4) a fourth probe comprising a sequence selected from the group consisting of TTTTTTTTTTTTTCGAAAAAAACAGCCAA (SEQ ID NO:192), TTTTTTTTTTTTTTCGCGAAAAAAACAGC (SEQ ID NO:194), TTTTTTTTTTTTTGTGTACCGCCGGAAGC (SEQ ID NO: 196), TTTTTTTTTTTTTTACCGCCGGAAGCACC (SEQ ID NO: 198), and TTTTTTTTTTTTTTACAGCCAAGTGTACCG (SEQ ID NO:200);
wherein each of the four probes is coupled to a microcarrier with a different identifier.

91. The method of any one of claims 83-90, wherein the first primer specific for one or more mutations in the ALK gene comprises the sequence AGTTGGGGTTGTAGTCGGTCAT (SEQ ID NO:363) or GAAGCCTCCCTGGATCTCC (SEQ ID NO:364).

92. The method of any one of claims 83-91, wherein the second primer specific for one or more mutations in the ALK gene comprises a sequence selected from the group consisting of TATGGAGCAAAACTACTGTAGAGCC (SEQ ID NO:357), CCAGCTACATCACACACCTTGACT (SEQ ID NO:358), and TAATACCAAAAGTTACCAAAACTGCA (SEQ ID NO:359).

93. The method of any one of claims 79-92, wherein the one or more mutations in the ROS gene comprise an ROS fusion gene selected from the group consisting of CD74-ROS, and SLC34A2-ROS.

94. The method of claim 93, wherein the first primer is specific for a region of the CD74, or SLC34A2, and the second primer is specific for a region of the ROS locus.

95. The method of claim 93, wherein the second primer is specific for a region of the CD74, or SLC34A2, locus, and the first primer is specific for a region of the ROS locus.

96. The method of claim 93, wherein the first primer is specific for a region of the CD74 or SLC34A2 locus, and the second primer is specific for a region of the ROS locus; or wherein the second primer is specific for a region of the CD74 or SLC34A2 locus, and the first primer is specific for a region of the ROS locus.

97. The method of any one of claims 93-96, wherein the one or more mutations in the ROS gene comprise one or more of CD74 E6:ROS E32, CD74 E6:ROS E34, SLC34A2 E4:ROS E32, and SLC34A2 E4:ROS E34 fusion genes.

98. The method of claim 97, wherein the one or more mutations in the ROS gene comprise CD74 E6:ROS E32, CD74 E6:ROS E34, SLC34A2 E4:ROS E32, and SLC34A2 E4:ROS E34 fusion genes.

99. The method of claim 98, wherein the probes specific for one or more mutations in the ROS gene comprise:
(1) a first probe comprising a sequence selected from the group consisting of ACTGACGCTCCACCGAAA (SEQ ID NO:201), CCACTGACGCTCCACCGA (SEQ ID NO:203), GCTGGAGTCCCAAATAAAC (SEQ ID NO:205), GGAGTCCCAAATAAACCAG (SEQ ID NO:207), and CACCGAAAGCTGGAGTCCC (SEQ ID NO:209);
(2) a second probe comprising a sequence selected from the group consisting of CCGAAAGATGATTTT (SEQ ID NO:211), GACGCTCCACCGAAA (SEQ ID NO:213), ACTGACGCTCCACCGA (SEQ ID NO:215), GATGATTTTTGGATA (SEQ ID NO:217), and TGATTTTTGGATACCA (SEQ ID NO: 219);
(3) a third probe comprising a sequence selected from the group consisting of AGCGCCTTCCAGCTGGTTGGA (SEQ ID NO:221), CTGGTTGGAGCTGGAGTCCC (SEQ ID NO:223), AGTAGCGCCTTCCAGCTGGTTG (SEQ ID NO:225), GCTGGAGTCCCAAATAAACCA (SEQ ID NO:227), and GGAGTCCCAAATAAACCAGG (SEQ ID NO:229); and
(4) a fourth probe comprising a sequence selected from the group consisting of GCGCCTTCCAGCTGGTTG (SEQ ID NO:231), GTAGCGCCTTCCAGCTGGT (SEQ ID NO:233), TGGTTGGAGATGATTTTT (SEQ ID NO:235), GATGATTTTTGGATACCAG (SEQ ID NO:237), and TGATTTTTGGATACCA (SEQ ID NO:239);
wherein each of the four probes is coupled to a microcarrier with a different identifier.

100. The method of claim 99, wherein each of the four probes further comprises eight nucleotides at the 5′ end, and wherein the eight nucleotides at the 5′ end are adenine or thymine nucleotides.

101. The method of claim 100, wherein the probes specific for one or more mutations in the ROS gene comprise:
(1) a first probe comprising a sequence selected from the group consisting of TTTTTTTTTTTACTGACGCTCCACCGAAA (SEQ ID NO:202), TTTTTTTTTTTCCACTGACGCTCCACCGA (SEQ ID NO:204), TTTTTTTTTTTGCTGGAGTCCCAAATAAAC (SEQ ID NO:206), TTTTTTTTTTTGGAGTCCCAAATAAACCAG (SEQ ID NO:208), and TTTTTTTTTTTCACCGAAAGCTGGAGTCCC (SEQ ID NO:210);
(2) a second probe comprising a sequence selected from the group consisting of TTTTTTTTTTTTCCGAAAGATGATTTT (SEQ ID NO:212), TTTTTTTTTTTTGACGCTCCACCGAAA (SEQ ID NO:214), TTTTTTTTTTTTACTGACGCTCCACCGA (SEQ ID NO:216), TTTTTTTTTTTTGATGATTTTTGGATA (SEQ ID NO:218), and TTTTTTTTTTTTTGATTTTTGGATACCA (SEQ ID NO:220);
(3) a third probe comprising a sequence selected from the group consisting of TTTTTTTTTTTTAGCGCCTTCCAGCTGGTTGGA (SEQ ID NO:222), TTTTTTTTTTTTCTGGTTGGAGCTGGAGTCCC (SEQ ID NO:224), TTTTTTTTTTTTAGTAGCGCCTTCCAGCTGGTTG (SEQ ID NO:226), TTTTTTTTTTTTGCTGGAGTCCCAAATAAACCA (SEQ ID NO:228), and TTTTTTTTTTTTGGAGTCCCAAATAAACCAGG (SEQ ID NO:230); and
(4) a fourth probe comprising a sequence selected from the group consisting of TTTTTTTTTTGCGCCTTCCAGCTGGTTG (SEQ ID NO:232), TTTTTTTTTTGTAGCGCCTTCCAGCTGGT (SEQ ID NO:234), TTTTTTTTTTTGGTTGGAGATGATTTTT (SEQ ID NO:236), TTTTTTTTTTGATGATTTTTGGATACCAG (SEQ ID NO:238), and TTTTTTTTTTTGATTTTTGGATACCA (SEQ ID NO:240);
wherein each of the four probes is coupled to a microcarrier with a different identifier.

102. The method of any one of claims 93-101, wherein the first primer specific for one or more mutations in the ROS gene comprises the sequence AATTCAATACATACTATCAGCTTTCTCCCACTGTATTGAA (SEQ ID NO:21) or AATATTTCTGGTACGAGTGGGATTGTAACAACCAGAAATA (SEQ ID NO:22).

103. The method of any one of claims 93-102, wherein the second primer specific for one or more mutations in the ROS gene comprises the sequence GGAGTGCCATCGCTGTTTGAAATGAGCAGGCACT (SEQ ID NO:19) or TACAGCCCTGGATATTCTTAGTAGCGC (SEQ ID NO:20).

104. The method of any one of claims 79-103, wherein the one or more mutations in the RET gene comprise a RET fusion gene selected from the group consisting of KIF5B-RET.

105. The method of claim 104, wherein the first primer is specific for a region of the KIF5B, or CCDC6 locus, and the second primer is specific for a region of the RET locus.

106. The method of claim 104, wherein the second primer is specific for a region of the KIF5B, or CCDC6 locus, and the first primer is specific for a region of the RET locus.

107. The method of claim 104, wherein the first primer is specific for a region of the KIF5B locus, and the second primer is specific for a region of the RET locus; or wherein the second primer is specific for a region of the KIF5B locus, and the first primer is specific for a region of the RET locus.

108. The method of any one of claims 104-107, wherein the one or more mutations in the RET gene comprise one or more of KIF5B E15:RET E11, KIF5B E15:RET E12, KIF5B E16:RET E12, KIF5B E22:RET E12, KIF5B E23:RET E12, and CCDC6 E1:RET E12 fusion genes.

109. The method of claim 108, wherein the one or more mutations in the RET gene comprise KIF5B E15:RET E11, KIF5B E15:RET E12, KIF5B E16:RET E12, KIF5B E22:RET E12, and KIF5B E23:RET E12 fusion genes.

110. The method of claim 109, wherein the probes specific for one or more mutations in the RET gene comprise:
(1) a first probe comprising a sequence selected from the group consisting of GTGGGAAATAATGATGTAAA (SEQ ID NO:241), CTGTGGGAAATAATGATGTA (SEQ ID NO:243), GATCCACTGTGCGACGAGCT (SEQ ID NO:245), TGATGTAAAGATCCACTGTG (SEQ ID NO:247), and TCCACTGTGCGACGAGCTGT (SEQ ID NO:249);
(2) a second probe comprising a sequence selected from the group consisting of TGGGAAATAATGATGTAAA (SEQ ID NO:251), CTGTGGGAAATAATGATGTA (SEQ ID NO:253), GGAGGATCCAAAGTGGGAAT (SEQ ID NO:255), GGATCCAAAGTGGGAATT (SEQ ID NO:257), and ATGATGTAAAGGAGGATCC (SEQ ID NO:259);
(3) a third probe comprising a sequence selected from the group consisting of CTTCGTATCTCTCAAGAGGAT (SEQ ID NO:481), GTATCTCTCAAGAGGATCCAA (SEQ ID NO:483), TTCGTATCTCTCAAGAG (SEQ ID NO:485), TCAAGAGGATCCAAA (SEQ ID NO:487), and TCTCTCAAGAGG (SEQ ID NO:489);
(4) a fourth probe comprising a sequence selected from the group consisting of GTTAAAAAGGAGGATCCAA (SEQ ID NO:491), ACAAGAGTTAAAAAGGAGGA (SEQ ID NO:493), AAGAGTTAAAAAGGAGGATC (SEQ ID NO:495), AAAAGGAGGATCCAAAG (SEQ ID NO:497), and AAGGAGGATCCAAAGTG (SEQ ID NO:499); and
(5) a fifth probe comprising a sequence selected from the group consisting of AAACAGGAGGATCCAAA (SEQ ID NO:501), AAGTGCACAAACAGGAGG (SEQ ID NO:503), GTGCACAAACAGGAGGATC (SEQ ID NO:505), CACAAACAGGAGGAT (SEQ ID NO:507), and AACAGGAGGATCCAAA (SEQ ID NO:509);
wherein each of the five probes is coupled to a microcarrier with a different identifier.

111. The method of claim 110, wherein each of the four probes further comprises eight nucleotides at the 5′ end, and wherein the eight nucleotides at the 5′ end are adenine or thymine nucleotides.

112. The method of claim 111, wherein the probes specific for one or more mutations in the RET gene comprise:
(1) a first probe comprising a sequence selected from the group consisting of TTTTTTTTTTGTGGGAAATAATGATGTAAA (SEQ ID NO:242), TTTTTTTTTTCTGTGGGAAATAATGATGTA (SEQ ID NO:244), TTTTTTTTTTGATCCACTGTGCGACGAGCT (SEQ ID NO:246), TTTTTTTTTTTGATGTAAAGATCCACTGTG (SEQ ID NO:248), and TTTTTTTTTTTCCACTGTGCGACGAGCTGT (SEQ ID NO:250);
(2) a second probe comprising a sequence selected from the group consisting of TTTTTTTTTTGGGAAATAATGATGTAAA (SEQ ID NO:252), TTTTTTTTTCTGTGGGAAATAATGATGTA (SEQ ID NO:254), TTTTTTTTTGGAGGATCCAAAGTGGGAAT (SEQ ID NO:256), TTTTTTTTTGGATCCAAAGTGGGAATT (SEQ ID NO:258), and TTTTTTTTATGATGTAAAGGAGGATCC (SEQ ID NO:260);
(3) a third probe comprising a sequence selected from the group consisting of TTTTTTTTTCTTCGTATCTCTCAAGAGGAT (SEQ ID NO:482), TTTTTTTTTGTATCTCTCAAGAGGATCCAA (SEQ ID NO:484), TTTTTTTTTTTCGTATCTCTCAAGAG (SEQ ID NO:486), TTTTTTTTTTCAAGAGGATCCAAA (SEQ ID NO:488), and TTTTTTTTTTCTCTCAAGAGG (SEQ ID NO:490);
(4) a fourth probe comprising a sequence selected from the group consisting of TTTTTTTTTGTTAAAAAGGAGGATCCAA (SEQ ID NO:492), TTTTTTTTACAAGAGTTAAAAAGGAGGA (SEQ ID NO:494), TTATTATTAAGAGTTAAAAAGGAGGATC (SEQ ID NO:811), TTTTTTTTAAAAGGAGGATCCAAAG (SEQ ID NO:498), and TTTTTTTTAAGGAGGATCCAAAGTG (SEQ ID NO:500); and
(5) a fifth probe comprising a sequence selected from the group consisting of TTTTTTTTAAACAGGAGGATCCAAA (SEQ ID NO:502), TTTTTATTAAGTGCACAAACAGGAGG (SEQ ID NO:504), TATTATTATGTGCACAAACAGGAGGATC (SEQ ID NO:506), TATTTTTTCACAAACAGGAGGAT (SEQ ID NO:508), and TTTTATTTAACAGGAGGATCCAAA (SEQ ID NO:510);
wherein each of the five probes is coupled to a microcarrier with a different identifier.

113. The method of any one of claims 103-112, wherein the first primer specific for one or more mutations in the RET gene comprises the sequence GTGATCGCACAGTAGGACAGCGGCTGCGATC (SEQ ID NO:26) or CTCTAGGAGATATCATTCCAAATTCGCCTTCTCCTAG (SEQ ID NO:27).

114. The method of any one of claims 106-113, wherein the second primer specific for one or more mutations in the RET gene comprises a sequence selected from the group consisting of TTTCTGGTGCTATGAGGAAATGACCAACCACCAGA (SEQ ID NO:23), AAGGAGTTAGCAGCATGTCAGC (SEQ ID NO:519), AACTTCAGACTTTACACAACCTGC (SEQ ID NO:520), and ATTGATTCTGATGACACCGGA (SEQ ID NO:521).

115. The method of any one of claims 79-114, wherein the one or more mutations in the NTRK1 gene comprise a CD74-NTRK1 fusion gene.

116. The method of claim 115, wherein the first primer is specific for a region of the CD74 locus, and the second primer is specific for a region of the NTRK1 locus.

117. The method of claim 115, wherein the second primer is specific for a region of the CD74 locus, and the first primer is specific for a region of the NTRK1 locus.

118. The method of any one of claims 115-117, wherein the one or more mutations in the NTRK1 gene comprise a CD74 E8:NTRK1 E12 fusion gene.

119. The method of claim 118, wherein the probe specific for one or more mutations in the NTRK1 gene comprises a sequence selected from the group consisting of CAGGATCTGGGCCCAGACA (SEQ ID NO:261), GATCTGGGCCCAGACACTA (SEQ ID NO:263), CCAGACACTAACAGCACAT (SEQ ID NO:265), GGGCCCAGACACTAACAGC (SEQ ID NO:267), and CTAACAGCACATCTGGAGA (SEQ ID NO:269).

120. The method of claim 119, wherein the probe specific for one or more mutations in the NTRK1 gene further comprises eight nucleotides at the 5′ end, and wherein the eight nucleotides at the 5′ end are adenine or thymine nucleotides.

121. The method of claim 120, wherein the probe specific for one or more mutations in the NTRK1 gene comprises a sequence selected from the group consisting of TTTTTTTTTTACAGGATCTGGGCCCAGACA (SEQ ID NO:262), TTTTTTTTTTAGATCTGGGCCCAGACACTA (SEQ ID NO:264), TTTTTTTTTTACCAGACACTAACAGCACAT (SEQ ID NO:266), TTTTTTTTTTAGGGCCCAGACACTAACAGC (SEQ ID NO:268), and TTTTTTTTTTACTAACAGCACATCTGGAGA (SEQ ID NO:270).

122. The method of any one of claims 115-120, wherein the first primer specific for one or more mutations in the NTRK1 gene comprises the sequence GGACGAAAATCCAGACCCCAAAAGGTGTTTCGT (SEQ ID NO:32).

123. The method of any one of claims 115-122, wherein the second primer specific for one or more mutations in the NTRK1 gene comprises the sequence AGAAGACGTGACAGGAACTGGAGGACCCGTCTT (SEQ ID NO:30).

124. The method of any one of claims 79-123, wherein the one or more mutations in the cMET gene results in exon skipping.

125. The method of claim 124, wherein the one or more mutations in the cMET gene results in skipping of exon 14.

126. The method of claim 125, wherein the probe specific for one or more mutations in the cMET gene comprises a sequence selected from the group consisting of AGAAAGCAAATTAAAGAT (SEQ ID NO:271), AGCAAATTAAAGATCAG (SEQ ID NO:273), AAATTAAAGATCAGTTTC (SEQ ID NO:275), AGATCAGTTTCCTAATTC (SEQ ID NO:277), and AAGATCAGTTTCCTAATT (SEQ ID NO:279).

127. The method of claim 126, wherein the probe specific for one or more mutations in the cMET gene further comprises eight nucleotides at the 5′ end, and wherein the eight nucleotides at the 5′ end are adenine or thymine nucleotides.

128. The method of claim 127, wherein the probe specific for one or more mutations in the cMET gene comprises a sequence selected from the group consisting of TTTTTTTTTTAGAAAGCAAATTAAAGAT (SEQ ID NO:272), TTTTTTTTTTAGCAAATTAAAGATCAG (SEQ ID NO:274), TTTTTTTTTTAAATTAAAGATCAGTTTC (SEQ ID NO:276), TTTTTTTTTTAGATCAGTTTCCTAATTC (SEQ ID NO:278), and TTTTTTTTTTAAGATCAGTTTCCTAATT (SEQ ID NO:280).

129. The method of any one of claims 124-128, wherein the first primer specific for one or more mutations in the cMET gene comprises the sequence GACAGTATTTTGCAGTAATGGACTGGATATATCAGA (SEQ ID NO:29).

130. The method of any one of claims 124-129, wherein the second primer specific for one or more mutations in the cMET gene comprises the sequence GAATTTCACAGGATTGATTGCTGGTGTTGTCTC (SEQ ID NO:28).

131. The method of any one of claims 79-130, wherein the sample is a blood, serum, or plasma sample.

132. The method of claim 131, wherein isolating RNA from the sample in (a) comprises isolating RNA from one or more of tumor-conditioned platelets, tumor exosomes, and circulating tumor cells (CTCs).

133. The method of any one of claims 79-132, wherein the method further comprises:
amplifying a positive control DNA sequence from the isolated RNA by reverse transcription-polymerase chain reaction (RT-PCR) in (b), wherein amplifying the positive control DNA sequence comprises:
(1) generating cDNA specific for the positive control sequence from the isolated RNA using a first primer specific for the positive control sequence, the isolated RNA, and a reverse transcriptase, and
(2) amplifying DNA specific for the positive control sequence by polymerase chain reaction (PCR) using the cDNA specific for the positive control sequence generated in (1), a DNA polymerase, the first primer, and a second primer specific for the positive control sequence that binds to a strand of the cDNA opposite the corresponding first primer and promotes strand extension in a direction opposite that promoted by the corresponding first primer;
hybridizing the amplified positive control gene sequence with a probe specific for the positive control gene sequence in (c), wherein the probe specific for the positive control gene sequence is coupled to a microcarrier with an identifier corresponding to a positive control;
detecting presence or absence of hybridization of the amplified positive control DNA sequence with the probe specific for the positive control gene sequence in (d); and
detecting the identifier corresponding to the positive control in (e).

134. The method of any one of claims 79-133, wherein the method further comprises:
detecting absence of hybridization of the amplified DNA with a microcarrier having an identifier corresponding to a negative control in (d), wherein the microcarrier with the identifier corresponding to the negative control comprises a probe that does not hybridize with the amplified DNA; and
detecting the identifier corresponding to the negative control in (e).

135. A method for detecting the presence of mutations in the genes, the method comprising:
(a) isolating DNA and RNA from a sample;
(b) amplifying the isolated DNA by polymerase chain reaction (PCR) using primer pairs specific for the loci of one or more DNA mutations in each of the KRAS, NRAS, PIK3CA, BRAF, EGFR AKT1, MEK1, and HER2 genes;
(c) amplifying DNA from the isolated RNA by reverse transcription-polymerase chain reaction (RT-PCR), wherein amplifying the DNA from the isolated RNA comprises:
(1) generating cDNA specific for each of the ALK, ROS, RET, NTRK1, and cMET genes from the isolated RNA using a first primer specific for each of the ALK, ROS, RET, NTRK1, and cMET genes, the isolated RNA, and a reverse transcriptase, and
(2) amplifying DNA specific for each of the ALK, ROS, RET, NTRK1, and cMET genes by polymerase chain reaction (PCR) using the cDNA generated in (c)(1), a DNA polymerase, the first primer, and a second primer specific for each of the ALK, ROS, RET, NTRK1, and cMET genes that binds to a strand of the cDNA opposite the corresponding first primer and promotes strand extension in a direction opposite that promoted by the corresponding first primer;
(d) hybridizing the DNA amplified by PCR in (b) with at least seven probes, said at least seven probes comprising one or more probes specific for a mutation in each of the KRAS, NRAS, PIK3CA, BRAF, EGFR, AKT1, MEK1, and HER2 genes, wherein each of said at least seven probes is coupled to a microcarrier, and wherein each of the microcarriers comprises an identifier corresponding to the probe coupled thereto;
(e) detecting presence or absence of hybridization of the DNA amplified by PCR in (b) with said at least seven probes, wherein hybridization between the amplified DNA and one of the probes indicates the presence of the mutation corresponding to the probe;
(f) hybridizing the DNA amplified by RT-PCR in (c) with at least five probes, said at least five probes comprising one or more probes specific for a mutation in each of the ALK, ROS, RET, NTRK1, and cMET genes, wherein each of said at least five probes is coupled to a microcarrier, and wherein each of the microcarriers comprises an identifier corresponding to the probe coupled thereto;
(g) detecting presence or absence of hybridization of the DNA amplified by RT-PCR in (c) with said at least five probes, wherein hybridization between the amplified DNA and one of the probes indicates the presence of the mutation corresponding to the probe;
(h) detecting the identifiers of the microcarriers; and
(i) correlating the detected identifiers of the microcarriers with the presence or absence of hybridization of the amplified DNA to the corresponding probes of the microcarriers detected in (e) and (g).

136. The method of claim 135, wherein (a) comprises:
isolating total RNA-rich plasma (TRRP) by centrifuging the sample, wherein the sample comprises whole blood or plasma;
subjecting the TRRP to one or more centrifugation steps to generate an RNA fraction and a cell-free DNA (cfDNA) fraction, wherein the RNA fraction comprises one or more of platelets, white blood cells, exosomes, circulating tumor cells, and free RNA;
isolating DNA from the cfDNA fraction; and
isolating RNA from the RNA fraction.

137. The method of any one of claims 1-136, wherein each of the primer pairs comprises a primer coupled to a detection reagent.

138. The method of claim 137, wherein the detection reagent comprises a fluorescent detection reagent, and wherein detecting the presence or absence of hybridization of the amplified DNA with said probes in (d) comprises fluorescence imaging of the fluorescent detection reagent.

139. The method of claim 137, wherein the detection reagent comprises biotin, and wherein detecting the presence or absence of hybridization of the amplified DNA with said probes in step (d) comprises:
(1) after hybridization in (c), contacting the microcarriers with streptavidin conjugated to a signal-emitting entity; and
(2) detecting a signal from the signal-emitting entity in association with the microcarriers.

140. The method of claim 139, wherein the signal-emitting entity comprises phycoerythrin (PE).

141. The method of any one of claims 1-140, wherein detecting the identifiers of the microcarriers in (e) comprises bright field imaging of the identifiers.

142. The method of any one of claims 1-141, wherein the identifiers of the microcarriers comprise digital barcodes.

143. The method of claim 142, wherein each of the microcarriers comprises:
(i) a first photopolymer layer;
(ii) a second photopolymer layer; and
(iii) an intermediate layer between the first layer and the second layer, the intermediate layer having an encoded pattern representing the identifier defined thereon, wherein the intermediate layer is partially substantially transmissive and partially substantially opaque to light, representing a code corresponding to the microcarrier, wherein the outermost surface of the microcarrier comprises a photoresist photopolymer, and said photoresist photopolymer is functionalized with the probe specific for the DNA mutation, and wherein said microcarrier has about the same density as water.

144. The method of any one of claims 1-141, wherein the identifiers of the microcarriers comprise analog codes.

145. The method of claim 144, wherein each of the microcarriers comprises:
(i) a substantially transparent polymer layer having a first surface and a second surface, the first and the second surfaces being parallel to each other;
(ii) a substantially non-transparent layer that constitutes a two-dimensional shape, wherein the substantially non-transparent layer is affixed to the first surface of the substantially transparent polymer layer and encloses a center portion of the substantially transparent polymer layer, wherein the two-dimensional shape of the substantially non-transparent layer represents an analog code, and wherein the analog code corresponds to the identifier; and
(iii) the probe specific for the mutation, wherein the probe is coupled to at least one of the first surface and the second surface of the substantially transparent polymer layer in at least the center portion of the substantially transparent polymer layer.

146. The method of claim 145, wherein each of the microcarriers further comprises an orientation indicator for orienting the analog code of the substantially non-transparent polymer layer.

147. The method of claim 145 or claim 146, wherein the polymer of the substantially transparent polymer layer comprises an epoxy-based polymer.

148. The method of claim 147, wherein the epoxy-based polymer is SU-8.

149. A kit comprising at least seven microcarriers, wherein each of said at least seven microcarriers comprises:
(i) a probe coupled to the microcarrier, wherein the probe is specific for a DNA mutation in the KRAS, PIK3CA, BRAF, EGFR, AKT1, MEK1, or HER2 gene; and
(ii) an identifier corresponding to the probe coupled thereto;
wherein the kit comprises at least one microcarrier comprising a probe specific for a DNA mutation in the KRAS gene, at least one microcarrier comprising a probe specific for a DNA mutation in the PIK3CA gene, at least one microcarrier comprising a probe specific for a DNA mutation in the BRAF gene, at least one microcarrier comprising a probe specific for a DNA mutation in the EGFR gene, at least one microcarrier comprising a probe specific for a DNA mutation in the AKT1 gene, at least one microcarrier comprising a probe specific for a DNA mutation in the MEK1 gene, and at least one microcarrier comprising a probe specific for a DNA mutation in the HER2 gene; and
wherein the KRAS, NRAS, PIK3CA, BRAF, EGFR, AKT1, MEK1, and HER2 genes are human genes.

150. The kit of claim 149, further comprising:
at least seven blocking nucleic acids, wherein each of said at least seven blocking nucleic acids hybridizes with a wild-type DNA locus corresponding with one of the DNA mutations in the KRAS, PIK3CA, BRAF, EGFR, AKT1, MEK1, or HER2 genes and prevents amplification of the wild-type DNA locus.

151. The kit of claim 150, wherein each of said at least seven blocking nucleic acids comprises:
a single-stranded oligonucleotide that hybridizes with the corresponding wild-type DNA locus; and
a 3′ terminal moiety that blocks extension from the single-stranded oligonucleotide.

152. The kit of claim 151, wherein the 3′ terminal moiety comprises one or more inverted deoxythymidines.

153. The kit of any one of claims 149-152, wherein each of said at least seven blocking nucleic acids comprises one or more modified nucleotides selected from the group consisting of locked nucleic acids (LNAs), peptide nucleic acids (PNAs), hexose nucleic acids (HNAs), threose nucleic acids (TNAs), glycol nucleic acids (GNAs), and cyclohexenyl nucleic acids (CeNAs).

154. The kit of any one of claims 149-153, wherein the DNA mutation in the KRAS gene comprises one or more DNA mutations encoding a G12D, G12V, or G12C mutated KRAS protein.

155. The kit of claim 154, wherein the DNA mutation in the KRAS gene comprises DNA mutations encoding G12D, G12V, and G12C mutated KRAS proteins.

156. The kit of claim 155, wherein the probes specific for the DNA mutation in the KRAS gene comprise:
(1) a first probe comprising a sequence selected from the group consisting of TAGTTGGAGCT (SEQ ID NO:38), TGTGGTAGTTG (SEQ ID NO:40), TGATGGCGTAG (SEQ ID NO:42), TGGAGCTGATGGC (SEQ ID NO:44), and GCGTAGGCAAG (SEQ ID NO:46);
(2) a second probe comprising a sequence selected from the group consisting of CTGTTGGCGTAGG (SEQ ID NO:48), GTAGTTGGAGCTG (SEQ ID NO:50), TGGAGCTGTTGGC (SEQ ID NO:52), TTGTGGTAGTTGG (SEQ ID NO:54), and GGCGTAGGCAAGA (SEQ ID NO:56); and
(3) a third probe comprising a sequence selected from the group consisting of TAGTTGGAGCTT (SEQ ID NO:58), GCGTAGGCAAGA (SEQ ID NO:60), GGAGCTTGTGGC (SEQ ID NO:62), TTGTGGCGTAGG (SEQ ID NO:64), and TGTGGTAGTTGG (SEQ ID NO: 66);
wherein each of the three probes is coupled to a microcarrier with a different identifier.

157. The kit of claim 156, wherein each of the three probes further comprises eight nucleotides at the 5′ end, and wherein the eight nucleotides at the 5′ end are adenine or thymine nucleotides.

158. The kit of claim 157, wherein the probes specific for the DNA mutation in the KRAS gene comprise:
(1) a first probe comprising a sequence selected from the group consisting of TTTTTTTTTTTTAATAGTTGGAGCT (SEQ ID NO:39), TTTTTTTTTTTTAATGTGGTAGTTG (SEQ ID NO:41), TTTTTTTTTTTTAATGATGGCGTAG (SEQ ID NO: 43), TTTTTTTTTTTATGGAGCTGATGGC (SEQ ID NO: 45), and TTTTTTTTTTTTAAGCGTAGGCAAG (SEQ ID NO:47);
(2) a second probe comprising a sequence selected from the group consisting of TTTTTTTTTTTACTGTTGGCGTAGG (SEQ ID NO:49), TTTTTTTTTTTAGTAGTTGGAGCTG (SEQ ID NO:51), TTTTTTTTTTTATGGAGCTGTTGGC (SEQ ID NO:53), TTTTTTTTTTTATTGTGGTAGTTGG (SEQ ID NO:55), and TTTTTTTTTTTAGGCGTAGGCAAGA (SEQ ID NO:57); and
(3) a third probe comprising a sequence selected from the group consisting of TTTTTTTTTTTAATAGTTGGAGCTT (SEQ ID NO:59), TTTTTTTTTTTAAGCGTAGGCAAGA (SEQ ID NO:61), TTTTTTTTTTTAAGGAGCTTGTGGC (SEQ ID NO: 63), TTTTTTTTTTTAATTGTGGCGTAGG (SEQ ID NO: 65), and TTTTTTTTTTTAATGTGGTAGTTGG (SEQ ID NO:67);
wherein each of the three probes is coupled to a microcarrier with a different identifier.

159. The kit of any one of claims 154-158, further comprising: a primer pair comprising the sequences GTACTGGTGGAGTATTTGATAGTG (SEQ ID NO:1) and CGTCAAGGCACTCTTGCCTAC (SEQ ID NO: 2).

160. The kit of any one of claims 154-159, further comprising: a blocking nucleic acid that hybridizes with a wild-type KRAS DNA locus corresponding with the KRAS DNA mutation and prevents amplification of the wild-type KRAS DNA locus, and wherein the blocking nucleic acid comprises the sequence TACGCCACCAGCT(invdT)ₙ, wherein n is 1, 2, or 3 (SEQ ID NO:281); TTGGAGCTGGTGGCGTA(invdT)ₙ, wherein n is 1, 2, or 3 (SEQ ID NO:282); GCTGGTGGCGTAGGCA(invdT)ₙ, wherein n is 1, 2, or 3 (SEQ ID NO:283); GCTGGTGGCGTAGGC(invdT)ₙ, wherein n is 1, 2, or 3 (SEQ ID NO:284); or TTGGAGCTGGTGGCGT(invdT)ₙ, wherein n is 1, 2, or 3 (SEQ ID NO:285); with italicized nucleic acids representing locked nucleic acids.

161. The kit of any one of claims 149-160, wherein the DNA mutation in the PIK3CA gene comprises one or more DNA mutations encoding an E542K or E545K mutated PIK3CA protein.

162. The kit of claim 161, wherein the DNA mutation in the PIK3CA gene comprises DNA mutations encoding E542K and E545K mutated PIK3CA proteins.

163. The kit of claim 162, wherein the probes specific for the DNA mutation in the PIK3CA gene comprise:
(1) a first probe comprising a sequence selected from the group consisting of GCTCAGTGATTTTAG (SEQ ID NO:87), TGCTCAGTGATTTT (SEQ ID NO: 89), GCTCAGTGATTTTAG (SEQ ID NO:91), CCTGCTCAGTGATTTTA (SEQ ID NO:93), and CTCAGTGATTTTAGA (SEQ ID NO:95); and
(2) a second probe comprising a sequence selected from the group consisting of TTCTCCTGCTTA (SEQ ID NO:97), CTCCTGCTTAGT (SEQ ID NO:99), TCTCCTGCTTAG (SEQ ID NO:101), TCCTGCTTAGTG (SEQ ID NO:103), and CTCCTGCTTAGTGA (SEQ ID NO:105);
wherein each of the two probes is coupled to a microcarrier with a different identifier.

164. The kit of claim 163, wherein each of the two probes further comprises eight nucleotides at the 5′ end, and wherein the eight nucleotides at the 5′ end are adenine or thymine nucleotides.

165. The kit of claim 164, wherein the probes specific for the DNA mutation in the PIK3CA gene comprise:
(1) a first probe comprising a sequence selected from the group consisting of TTTTTTTTTAGCTCAGTGATTTTAG (SEQ ID NO:88), TTTTTTTTTTGCTCAGTGATTTT (SEQ ID NO:90), TTTTTTTTTAGCTCAGTGATTTTAG (SEQ ID NO:92), TTTTTTTCCTGCTCAGTGATTTTA (SEQ ID NO:94), and TTTTTTTTTTTCTCAGTGATTTTAGA (SEQ ID NO: 96); and
(2) a second probe comprising a sequence selected from the group consisting of TTTTTTTTTTTTTCTCCTGCTTA (SEQ ID NO:98), TTTTTTTTTTTTTCTCCTGCTTAGT (SEQ ID NO:100), TTTTTTTTTTTATCTCCTGCTTAG (SEQ ID NO:102), TTTTTTTTTTTTTTTCCTGCTTAGTG (SEQ ID NO:104), and TTTTTTTTTTTTTCTCCTGCTTAGTGA (SEQ ID NO:106);
wherein each of the three probes is coupled to a microcarrier with a different identifier.

166. The kit of any one of claims 161-165, further comprising: a primer pair comprising the sequences CAATTTCTACAAGAGATCCTCTCTCT (SEQ ID NO:5) and CTCCATTTTAGCACTTACCTGTGAC (SEQ ID NO:6).

167. The kit of any one of claims 161-166, further comprising: a blocking nucleic acid that hybridizes with a wild-type PIK3CA DNA locus corresponding with the PIK3CA DNA mutation and prevents amplification of the wild-type PIK3CA DNA locus, and the blocking nucleic acid comprises the sequence CTGAAATCACTGAGCAGG(invdT)ₙ, wherein n is 1, 2, or 3 (SEQ ID NO:291); TCTCTGAAATCACTGAGCAGG(invdT)ₙ, wherein n is 1, 2, or 3 (SEQ ID NO:292); TCTCTGAAATCACTGAGCAGG(invdT)ₙ, wherein n is 1, 2, or 3 (SEQ ID NO:293); TCTCTGAAATCACTGAGCAGG(invdT)ₙ, wherein n is 1, 2, or 3 (SEQ ID NO:294); or TCTCTGAATTCACTGAGCAGG(invdT)ₙ, wherein n is 1, 2, or 3 (SEQ ID NO:295); with italicized nucleic acids representing locked nucleic acids.

168. The kit of any one of claims 149-167, wherein the DNA mutation in the PIK3CA gene comprises a DNA mutation encoding an H1047R mutated PIK3CA protein.

169. The kit of claim 168, wherein the probe specific for the DNA mutation in the PIK3CA gene comprises a sequence selected from the group consisting of GATGCACGTCATG (SEQ ID NO:107), TGAATGATGCACG (SEQ ID NO:109), TGATGCACGTC (SEQ ID NO:111), AATGATGCACGTCA (SEQ ID NO:113), and AATGATGCACGTC (SEQ ID NO:115).

170. The kit of claim 169, wherein the probe further comprises eight nucleotides at the 5′ end, and wherein the eight nucleotides at the 5′ end are adenine or thymine nucleotides.

171. The kit of claim 170, wherein the probe specific for the DNA mutation in the PIK3CA gene comprises a sequence selected from the group consisting of TTTTTTTTTTTTTTTGATGCACGTCATG (SEQ ID NO:108), TTTTTTTTTTTGAATGATGCACG (SEQ ID NO:110), TTTTTTTTTTTTTGATGCACGTC (SEQ ID NO:112), TTTTTTTTTTTTAATGATGCACGTCA (SEQ ID NO:114), and TTTTTTTTTTTTAATGATGCACGTC (SEQ ID NO:116).

172. The kit of any one of claims 168-171, further comprising: a primer pair comprising the sequences ACCCTAGCCTTAGATAAAACTGAGC (SEQ ID NO:7) and TTTGTTGTCCAGCCACCATGA (SEQ ID NO: 8).

173. The kit of any one of claims 168-172, further comprising: a blocking nucleic acid that hybridizes with a wild-type PIK3CA DNA locus corresponding with the PIK3CA DNA mutation and prevents amplification of the wild-type PIK3CA DNA locus, and wherein the blocking nucleic acid comprises the sequence CACCATGATGTGCAT(invdT)ₙ, wherein n is 1, 2, or 3 (SEQ ID NO:296); CCACCATGATGTGCAT(invdT)ₙ, wherein n is 1, 2, or 3 (SEQ ID NO:297); CACCATGATGTGCAT(invdT)ₙ, wherein n is 1, 2, or 3 (SEQ ID NO:298); CCACCATGATGTGCATCA(invdT)ₙ, wherein n is 1, 2, or 3 (SEQ ID NO:299); or CATGATGTGCA(invdT)ₙ, wherein n is 1, 2, or 3 (SEQ ID NO:300); with italicized nucleic acids representing locked nucleic acids.

174. The kit of any one of claims 149-173, wherein the DNA mutation in the BRAF gene comprises a DNA mutation encoding a V600E mutated BRAF protein.

175. The kit of claim 174, wherein the probe specific for the DNA mutation in the BRAF gene comprises a sequence selected from the group consisting of TTTGGTCTAGCTACAGA (SEQ ID NO:79), CTACAGAGAAATCTCGA (SEQ ID NO:81), GTGATTTTGGTCTAGCT (SEQ ID NO:83), and TCTAGCTACAGAGAAAT (SEQ ID NO:85).

176. The kit of claim 175, wherein the probe specific for one or more DNA mutations in the BRAF gene further comprises eight nucleotides at the 5′ end, and wherein the eight nucleotides at the 5′ end are adenine or thymine nucleotides.

177. The kit of claim 176, wherein the probe specific for the DNA mutation in the BRAF gene comprises a sequence selected from the group consisting of TTTTTTAATTGAGAAATCTCGATGGAG (SEQ ID NO:78), TTTTTTAATTTTTGGTCTAGCTACAGA (SEQ ID NO:80), TTTTTTAATTCTACAGAGAAATCTCGA (SEQ ID NO:82), TTTTTTAATTGTGATTTTGGTCTAGCT (SEQ ID NO:84), and TTTTTTAATTTCTAGCTACAGAGAAAT (SEQ ID NO:86).

178. The kit of any one of claims 174-177, further comprising: a primer pair comprising the sequences ATAGCCTCAATTCTTACCATCCACAAAATG (SEQ ID NO:9) and CAGATATATTTCTTCATGAAGACCTCACAGTAA (SEQ ID NO:10).

179. The kit of any one of claims 174-178, further comprising: a blocking nucleic acid that hybridizes with a wild-type BRAF DNA locus corresponding with the BRAF DNA mutation and prevents amplification of the wild-type BRAF DNA locus, and wherein the blocking nucleic acid comprises the sequence GAGATTTCACTGTAGC(invdT)ₙ, wherein n is 1, 2, or 3 (SEQ ID NO:301); GAGATTTCACTGTAGC(invdT)ₙ, wherein n is 1, 2, or 3 (SEQ ID NO:302); GAGATTCACTGTAGC(invdT)ₙ, wherein n is 1, 2, or 3 (SEQ ID NO:303); GAGATTTCACTGTAGC(invdT)ₙ, wherein n is 1, 2, or 3 (SEQ ID NO:304); or GAGATTTCACTGTAGC(invdT)ₙ, wherein n is 1, 2, or 3 (SEQ ID NO:305); with italicized nucleic acids representing locked nucleic acids.

180. The kit of any one of claims 149-179, wherein the DNA mutation in the EGFR gene comprises a DNA mutation encoding a G719A mutated EGFR protein.

181. The kit of claim 180, wherein the probe specific for the DNA mutation in the EGFR gene comprises a sequence selected from the group consisting of TCAAAGTGCTGGCCTC (SEQ ID NO:117), AGATCAAAGTGCTGGCCTCCG (SEQ ID NO:119), AAAGTGCTGGCCT (SEQ ID NO:121), AGTGCTGGCCT (SEQ ID NO:123), and AAGTGCTGGCCTC (SEQ ID NO:125).

182. The kit of claim 181, wherein the probe specific for the DNA mutation in the EGFR gene further comprises eight nucleotides at the 5′ end, and wherein the eight nucleotides at the 5′ end are adenine or thymine nucleotides.

183. The kit of claim 182, wherein the probe specific for the DNA mutation in the EGFR gene comprises a sequence selected from the group consisting of TTTTTTTTTTCAAAGTGCTGGCCTC (SEQ ID NO:118), TTTTTTAGATCAAAGTGCTGGCCTCCG (SEQ ID NO:120), TTTTTTTTTTTAAAGTGCTGGCCT (SEQ ID NO: 122), TTTTTTTTTTTTTAGTGCTGGCCT (SEQ ID NO:124), and TTTTTTTTTTTTAAGTGCTGGCCTC (SEQ ID NO: 126).

184. The kit of any one of claims 180-183, further comprising: a primer pair comprising the sequences CTTGTGGAGCCTCTTACACCC (SEQ ID NO:11) and TGCCGAACGCACCGGA (SEQ ID NO:12).

185. The kit of any one of claims 180-184, further comprising: a blocking nucleic acid that hybridizes with a wild-type EGFR DNA locus corresponding with the EGFR DNA mutation and prevents amplification of the wild-type EGFR DNA locus, and wherein the blocking nucleic acid comprises the sequenceCGGAGCCCAGCACTTTGA (invdT)ₙ, wherein n is 1, 2, or 3 (SEQ ID NO:306); CGCACCGGAGCCCAGCACT (invdT)ₙ, wherein n is 1, 2, or 3 (SEQ ID NO:307); GAGCCCAGCAC (invdT)ₙ, wherein n is 1, 2, or 3 (SEQ ID NO:308); CGCACCGGAGCCCAGCAC (invdT)ₙ, wherein n is 1, 2, or 3 (SEQ ID NO:309); or CGCACCGGAGCCCAGCACTTA (invdT)ₙ, wherein n is 1, 2, or 3 (SEQ ID NO:310); with italicized nucleic acids representing locked nucleic acids.

186. The kit of any one of claims 149-185, wherein the DNA mutation in the EGFR gene comprises a DNA mutation encoding an E746_A750del mutated EGFR protein.

187. The kit of claim 186, wherein the probe specific for the DNA mutation in the EGFR gene comprises:
(1) a first probe comprising a sequence selected from the group consisting of AATCAAAACATCTCCGAAAG (SEQ ID NO:128), CAAAACATCTCCG (SEQ ID NO:130), AACATCTCCG (SEQ ID NO:132), and AAACATCTCCGAAAGCC (SEQ ID NO:134); and
(2) a second probe comprising a sequence selected from the group consisting of AATCAAGACATCTCCGA (SEQ ID NO:136), GCAATCAAGACATCTCCGA (SEQ ID NO:138), AATCAAGACATCTC (SEQ ID NO:140), AATCAAGACATCTCCGAAAGC (SEQ ID NO:142), and CAAGACATCTCCGA (SEQ ID NO:144);
wherein each of the two probes is coupled to a microcarrier with a different identifier.

188. The kit of claim 187, wherein each of the two probes further comprises eight nucleotides at the 5′ end, and wherein the eight nucleotides at the 5′ end are adenine or thymine nucleotides.

189. The kit of claim 188, wherein the probe specific for the DNA mutation in the EGFR gene comprises:
(1) a first probe comprising a sequence selected from the group consisting of TTTTTTTTTAATCAAAACATCTCCG (SEQ ID NO:127), TTTTTTTTTAATCAAAACATCTCCGAAAG (SEQ ID NO:129), TTTTTTTTTTTACAAAACATCTCCG (SEQ ID NO:131), TTTTTTTTTTTTTTTAACATCTCCG (SEQ ID NO:133), and TTTTTTTTTTTTTTAAACATCTCCGAAAGCC (SEQ ID NO:135); and
(2) a second probe comprising a sequence selected from the group consisting of TTTTTTTTAATCAAGACATCTCCGA (SEQ ID NO:137), TTTTTTGCAATCAAGACATCTCCGA (SEQ ID NO:139), TTTTTTTTAATCAAGACATCTC (SEQ ID NO:141), TTTTTTTTAATCAAGACATCTCCGAAAGC (SEQ ID NO:143), and TTTTTTTTTTTCAAGACATCTCCGA (SEQ ID NO: 145);
wherein each of the two probes is coupled to a microcarrier with a different identifier.

190. The kit of any one of claims 186-189, further comprising: a primer pair comprising the sequences GCCAGTTAACGTCTTCCTTCTC (SEQ ID NO: 13) and ATCGAGGATTTCCTTGTTGGCTT (SEQ ID NO: 14).

191. The kit of any one of claims 186-190, further comprising: a blocking nucleic acid that hybridizes with a wild-type EGFR DNA locus corresponding with the EGFR DNA mutation and prevents amplification of the wild-type EGFR DNA locus, and wherein the blocking nucleic acid comprises the sequence CGGAGATGTTGCTTCTCTTAATTCC(invdT)ₙ, wherein n is 1, 2, or 3 (SEQ ID NO:311); CGGAGATGTTGCTTCTCT(invdT)ₙ, wherein n is 1, 2, or 3 (SEQ ID NO:312); GTTGCTTCTCTTAATTCC(invdT)ₙ, wherein n is 1, 2, or 3 (SEQ ID NO:313); ATGTTGCTTCTCT(invdT)ₙ, wherein n is 1, 2, or 3 (SEQ ID NO:314); or TTGCTTCTCTTA(invdT)ₙ, wherein n is 1, 2, or 3 (SEQ ID NO:315); with italicized nucleic acids representing locked nucleic acids.

192. The kit of any one of claims 149-191, wherein the DNA mutation in the EGFR gene comprises one or more DNA mutations encoding a T790M, C797S, S768I, V769_D770insASV, H773_V774insH, D770_N771insG, or D770_N771insSVD mutated EGFR protein.

193. The kit of claim 192, wherein the DNA mutation in the EGFR gene comprises DNA mutations encoding T790M, C797S, S768I, V769_D770insASV, H773_V774insH, D770_N771insG, and D770_N771insSVD mutated EGFR proteins.

194. The kit of claim 193, wherein the probe specific for the DNA mutation in the EGFR gene comprises:
(1) a first probe comprising a sequence selected from the group consisting of GAGATGCATGATGA (SEQ ID NO:146), TGAGATGCATGATGAG (SEQ ID NO:147), ATGAGATGCATGATGAG (SEQ ID NO:148), TGAGCTGCATGATGA (SEQ ID NO:149), and CATGAGATGCATGATGA (SEQ ID NO:150);
(2) a second probe comprising a sequence selected from the group consisting of CCAGGAGGCTGCCG (SEQ ID NO:461), CAGGAGGCTGCCGA (SEQ ID NO:463), TCCAGGAGGCTGCC (SEQ ID NO:465), CCAGGAGGCTGCC (SEQ ID NO:467), and CAGGAGGCTGCC (SEQ ID NO:469);
(3) a third probe comprising a sequence selected from the group consisting of CCAGGAGGGAGCC (SEQ ID NO:471), CCAGGAGGGAGCCG (SEQ ID NO:473), TCCAGGAGGGAGCC (SEQ ID NO:475), CAGGAGGGAGCCG (SEQ ID NO:477), and CAGGAGGGAGCCGA (SEQ ID NO:479);
(4) a fourth probe comprising a sequence selected from the group consisting of ATGGCCATCTTGG (SEQ ID NO:421), GGCCATCTTGGA (SEQ ID NO:423), GATGGCCATCTTG (SEQ ID NO:425), TGATGGCCATCTTG (SEQ ID NO:427), and TGGCCATCTTGG (SEQ ID NO:429);
(5) a fifth probe comprising a sequence selected from the group consisting of GTGATGGCCGG (SEQ ID NO:431), TGATGGCCGGCG (SEQ ID NO:433), GTGATGGCCGGCGT (SEQ ID NO:435), GATGGCCGGCGT (SEQ ID NO:437), and GATGGCCCGCGTG (SEQ ID NO:439);
(6) a sixth probe comprising a sequence selected from the group consisting of AACCCCCATCACGT (SEQ ID NO:441), GACAACCCCCATCACG (SEQ ID NO:443), CGTGGACAACCCCCATCA (SEQ ID NO:445), CCCATCACGTGT (SEQ ID NO:447), and TGGACAACCCCCATCAC (SEQ ID NO:449); and
(7) a seventh probe comprising a sequence selected from the group consisting of GCCAGCGTGGACGG (SEQ ID NO:451), CGTGGACGGTAACC (SEQ ID NO:453), GACGGTAACCCCC (SEQ ID NO:455), CCAGCGTGGACGGT (SEQ ID NO:457), and GCCAGCGTGGACGGTA (SEQ ID NO: 459);
wherein each of the seven probes is coupled to a microcarrier with a different identifier.

195. The kit of claim 194, wherein each of the seven probes specific for the DNA mutation in the EGFR gene further comprises eight nucleotides at the 5′ end, and wherein the eight nucleotides at the 5′ end are adenine or thymine nucleotides.

196. The kit of claim 195, wherein the probe specific for the DNA mutation in the EGFR gene comprises:
(1) a first probe comprising a sequence selected from the group consisting of TTTTTTTTTTTGAGATGCATGATGA (SEQ ID NO:352), TTTTTTTTTTGAGATGCATGATGAG (SEQ ID NO:353), TTTTTTTTATGAGATGCATGATGAG (SEQ ID NO:354), TTTTTTTTTTTGAGCTGCATGATGA (SEQ ID NO:355), and TTTTTTTTCATGAGATGCATGATGA (SEQ ID NO:356);
(2) a second probe comprising a sequence selected from the group consisting of TTTTTTTTTTTACCAGGAGGCTGCCG (SEQ ID NO:462), TTTTTTTTTTTACAGGAGGCTGCCGA (SEQ ID NO:464), TTTTTTTTTTTATCCAGGAGGCTGCC (SEQ ID NO:466), TTTTTTTTTTTACCAGGAGGCTGCC (SEQ ID NO:468), and TTTTTTTTTTTACAGGAGGCTGCC (SEQ ID NO:470);
(3) a third probe comprising a sequence selected from the group consisting of TTTTTTTTTTTACCAGGAGGGAGCC (SEQ ID NO:472), TTTTTTTTTTTACCAGGAGGGAGCCG (SEQ ID NO:474), TTTTTTTTTTTATCCAGGAGGGAGCC (SEQ ID NO:476), TTTTTTTTTTTACAGGAGGGAGCCG (SEQ ID NO:478), and TTTTTTTTTTTACAGGAGGGAGCCGA (SEQ ID NO:480);
(4) a fourth probe comprising a sequence selected from the group consisting of TTTTTTTTTATGGCCATCTTGG (SEQ ID NO:422), TTTTTTTTTTAGGCCATCTTGGA (SEQ ID NO:424), TTTTTTTAGATGGCCATCTTG (SEQ ID NO:426), TTTTTTTTGATGGCCATCTTG (SEQ ID NO:428), and TTTTTTTTTTTGGCCATCTTGG (SEQ ID NO:430);
(5) a fifth probe comprising a sequence selected from the group consisting of TTTTTTTTTTTGTGATGGCCGG (SEQ ID NO:432), TTTTTTTTTTTTTGATGGCCGGCG (SEQ ID NO:434), TTTTTTTTTTTGTGATGGCCGGCGT (SEQ ID NO:436), TTTTTTTTTTTTTGATGGCCGGCGT (SEQ ID NO:438), and TTTTTTTTTTTTTGATGGCCCGCGTG (SEQ ID NO:440);
(6) a sixth probe comprising a sequence selected from the group consisting of TTTTTTTTTTTAACCCCCATCACGT (SEQ ID NO:442), TTTFTTTTGACAACCCCCATCACG (SEQ ID NO:444), TTTTCGTGGACAACCCCCATCA (SEQ ID NO:446), TTTTTTTTTTTTCCCATCACGTGT (SEQ ID NO:448), and TTTTTTTTGGACAACCCCCATCAC (SEQ ID NO:450); and
(7) a seventh probe comprising a sequence selected from the group consisting of TTTTTTTTTTTGCCAGCGTGGACGG (SEQ ID NO:452), TTTTTTTTTTTCGTGGACGGTAACC (SEQ ID NO:454), TTTTTTTTTTTGACGGTAACCCCC (SEQ ID NO:456), TTTTTTTTTTCCAGCGTGGACGGT (SEQ ID NO:458), and TTTTTTTGCCAGCGTGGACGGTA (SEQ ID NO:460);
wherein each of the seven probes is coupled to a microcarrier with a different identifier.

197. The kit of any one of claims 192-196, further comprising: a primer pair comprising the sequences CCTCCACCGTGCAGATCATC (SEQ ID NO:15) and TTCCCTGATTACCTTTGCGAT (SEQ ID NO:16); a primer pair comprising the sequences CCACACTGACGTGCCTCT (SEQ ID NO:511) and GCACACGTAGGGGTTGTCCAAGA (SEQ ID NO:512); a primer pair comprising the sequences CCACACTGACGTGCCTCT (SEQ ID NO:513) and GTACACGCTGGCCACGCCG (SEQ ID NO:514); a primer pair comprising the sequences CCACACTGACGTGCCTCT (SEQ ID NO:515) and CAGGCGGCACACGTGAT (SEQ ID NO:516); and/or a primer pair comprising the sequences CCACACTGACGTGCCTCT (SEQ ID NO:517) and AGGCGGCACACGTGCGGGTTAC (SEQ ID NO:518).

198. The kit of any one of claims 192-197, further comprising: a blocking nucleic acid that hybridizes with a wild-type EGFR DNA locus corresponding with the EGFR DNA mutation and prevents amplification of the wild-type EGFR DNA locus, and wherein the blocking nucleic acid comprises the sequence CATCACGCAGCTCATG(invdT)ₙ, wherein n is 1, 2, or 3 (SEQ ID NO:316); TGCAGCTCATCACGCAGC(invdT)ₙ, wherein n is 1, 2, or 3 (SEQ ID NO: 317); TCATCACGCAGCTCAT(invdT)ₙ, wherein n is 1, 2, or 3 (SEQ ID NO:318); TCATCACGCAGC(invdT)ₙ, wherein n is 1, 2, or 3 (SEQ ID NO: 319); or CTCATCACGCAGC(invdT), wherein n is 1, 2, or 3 (SEQ ID NO:320); with italicized nucleic acids representing locked nucleic acids.

199. The kit of any one of claims 149-198, wherein the DNA mutation in the EGFR gene comprises a DNA mutation encoding an L858R mutated EGFR protein.

200. The kit of claim 199, wherein the probe specific for the DNA mutation in the EGFR gene comprises a sequence selected from the group consisting of ATTTTGGGCGGGCC (SEQ ID NO:151), TTGGGCGGGCCAAA (SEQ ID NO: 153), GCGGGCCAAACT (SEQ ID NO:155), GGGCGGGCCAAACT (SEQ ID NO: 157), and TGGGCGGGCCA (SEQ ID NO: 159).

201. The kit of claim 200, wherein the probe further comprises eight nucleotides at the 5′ end, and wherein the eight nucleotides at the 5′ end are adenine or thymine nucleotides.

202. The kit of claim 201, wherein the probe specific for the DNA mutation in the EGFR gene comprises a sequence selected from the group consisting of TTTTTTTATTTTGGGCGGGCC (SEQ ID NO:152), TTTTTTTTAATTGGGCGGGCCAAA (SEQ ID NO:154), TTTTTTTAAAAAAGCGGGCCAAACT (SEQ ID NO:156), TTTTTTTTAAAAGGGCGGGCCAAACT (SEQ ID NO:158), and TTTTTTTTAAATGGGCGGGCCA (SEQ ID NO:160).

203. The kit of any one of claims 199-202, further comprising: a primer pair comprising the sequences GGAGGACCGTCGCTTGG (SEQ ID NO:17) and TCTTTCTCTTCCGCACCCAG (SEQ ID NO: 18).

204. The kit of any one of claims 199-203, further comprising: a blocking nucleic acid that hybridizes with a wild-type EGFR DNA locus corresponding with the EGFR DNA mutation and prevents amplification of the wild-type EGFR DNA locus, and wherein the blocking nucleic acid comprises the sequence CCAGCAGTTTGGCCAGCCCT(invdT)ₙ, wherein n is 1, 2, or 3 (SEQ ID NO:321); CCAGCAGTTTGGCCAGCCCT(invdT)ₙ, wherein n is 1, 2, or 3 (SEQ ID NO:322); CCAGCAGTTTGGCCAGCCCT(invdT)ₙ, wherein n is 1, 2, or 3 (SEQ ID NO:323); AGCAGTTTGGCCAGCC(invdT)ₙ, wherein n is 1, 2, or 3 (SEQ ID NO:324); or CCAGCAGTTTGGCCAGCCCT(invdT)ₙ, wherein n is 1, 2, or 3 (SEQ ID NO:325); with italicized nucleic acids representing locked nucleic acids.

205. The kit of any one of claims 149-204, wherein the DNA mutation in the AKT1 gene comprises a DNA mutation encoding an E17K mutated AKT1 protein.

206. The kit of claim 205, wherein the probe specific for the DNA mutation in the AKT1 gene comprises a sequence selected from the group consisting of TGTAGGGAAGTACA (SEQ ID NO:370), TCTGTAGGGAAGTAC (SEQ ID NO:372), GTCTGTAGGGAAGTACAT (SEQ ID NO:374), CCGCACGTCTGTAGGGA (SEQ ID NO:376), and ACGTCTGTAGGGAAGTA (SEQ ID NO:378).

207. The kit of claim 206, wherein the probe specific for the DNA mutation in the AKT1 gene further comprises seven nucleotides at the 5′ end, and wherein the seven nucleotides at the 5′ end are adenine or thymine nucleotides.

208. The kit of claim 207, wherein the probe specific for the DNA mutation in the AKT1 gene comprises a sequence selected from the group consisting of TTTTTTTTTTTTTTGTAGGGAAGTACA (SEQ ID NO:371), TTTTTTTTTTTTCTGTAGGGAAGTAC (SEQ ID NO:373), TTTTTTTGTCTGTAGGGAAGTACAT (SEQ ID NO:375), TTTTTTTCCGCACGTCTGTAGGGA (SEQ ID NO:377), and TTTTTTTTACGTCTGTAGGGAAGTA (SEQ ID NO:379).

209. The kit of any one of claims 205-208, further comprising: a primer pair comprising the sequences GAGGGTCTGACGGGTAGAGTG (SEQ ID NO:380) and TGGCCGCCAGGTCTTGATGTA (SEQ ID NO:381).

210. The kit of any one of claims 205-209, further comprising: a blocking nucleic acid that hybridizes with a wild-type AKT1 DNA locus corresponding with the AKT1 DNA mutation and prevents amplification of the wild-type AKT1 DNA locus, and wherein the blocking nucleic acid comprises the sequence TGTACTCCCCTACA (invdT)ₙ, wherein n is 1, 2, or 3 (SEQ ID NO:382); GATGTACTCCCCT (invdT)ₙ, wherein n is 1, 2, or 3 (SEQ ID NO:383); ATGTACTCCCCTAC (invdT)ₙ, wherein n is 1, 2, or 3 (SEQ ID NO:384); GTACTCCCCTACA (invdT)ₙ, wherein n is 1, 2, or 3 (SEQ ID NO:385); or GATGTACTCCCCTACA (invdT)ₙ, wherein n is 1, 2, or 3 (SEQ ID NO:386); with italicized nucleic acids representing locked nucleic acids.

211. The kit of any one of claims 149-210, wherein the DNA mutation in the MEK1 gene comprises a DNA mutation encoding a K57N mutated MEK1 protein.

212. The kit of claim 211, wherein the probe specific for the DNA mutation in the MEK1 gene comprises a sequence selected from the group consisting of TTACCCAGAATCAGAA (SEQ ID NO:387), CCAGAATCAGAAGGTG (SEQ ID NO:389), TTCTTACCCAGAATCA (SEQ ID NO:391), CCTTTCTTACCCAGAATC (SEQ ID NO:393), and CAGAATCAGAAGGTGG (SEQ ID NO:395).

213. The kit of claim 212, wherein the probe specific for the DNA mutation in the MEK1 gene further comprises eight nucleotides at the 5′ end, and wherein the eight nucleotides at the 5′ end are adenine or thymine nucleotides.

214. The kit of claim 213, wherein the probe specific for the DNA mutation in the MEK1 gene comprises a sequence selected from the group consisting of TTTTTAAATTTACCCAGAATCAGAA (SEQ ID NO:388), TTTTTAAATCCAGAATCAGAAGGTG (SEQ ID NO:390), TTTTTAAATTTCTTACCCAGAATCA (SEQ ID NO:392), TTTTTAAATCCTTTCTTACCCAGAATC (SEQ ID NO:394), and TTTTTAAATCAGAATCAGAAGGTGG (SEQ ID NO:396).

215. The kit of any one of claims 211-214, further comprising: a primer pair comprising the sequences CTTGATGAGCAGCAGCGAAA (SEQ ID NO:397) and CCTTCAGTTCTCCCACCTTCTG (SEQ ID NO:398).

216. The kit of any one of claims 211-215, further comprising: a blocking nucleic acid that hybridizes with a wild-typeMEK1 DNA locus corresponding with the MEK1 DNA mutation and prevents amplification of the wild-type MEK1 DNA locus, and wherein the blocking nucleic acid comprises the sequence TCTGCTTCTGGGTAAG (invdT)ₙ, wherein n is 1, 2, or 3 (SEQ ID NO:399); TTCTGCTTCTGGGTAAGA (invdT)ₙ, wherein n is 1, 2, or 3 (SEQ ID NO:400); CACCTTCTGCTTCTGGG (invdT)ₙ, wherein n is 1, 2, or 3 (SEQ ID NO:401); TCTGCTTCTGGGTA (invdT)ₙ, wherein n is 1, 2, or 3 (SEQ ID NO:402); or CACCTTCTGCTTCTGGGTAAGA (invdT)ₙ, wherein n is 1, 2, or 3 (SEQ ID NO:403); with italicized nucleic acids representing locked nucleic acids.

217. The kit of any one of claims 149-216, wherein the DNA mutation in the HER2 gene comprises a DNA mutation encoding an A775_G776insYVMA mutated HER2 protein.

218. The kit of claim 217, wherein the probe specific for the DNA mutation in the HER2 gene comprises a sequence selected from the group consisting of ATACGTGATGTCTTAC (SEQ ID NO:404), ACGTGATGGCTTACGT (SEQ ID NO:406), AAGCATACGTGATGGCT (SEQ ID NO:408), GCATACGTGATGGCTT (SEQ ID NO:410), and GCATACGTGATGGCTTA (SEQ ID NO:412).

219. The kit of claim 218, wherein the probe specific for the DNA mutation in the HER2 gene further comprises five nucleotides at the 5′ end, and wherein the five nucleotides at the 5′ end are adenine or thymine nucleotides.

220. The kit of claim 219, wherein the probe specific for the DNA mutation in the HER2 gene comprises a sequence selected from the group consisting of TTTTTTTTTATACGTGATGTCTTAC (SEQ ID NO:405), TTTTTTTTTTTACGTGATGGCTTACGT (SEQ ID NO:407), TTTTTAAGCATACGTGATGGCT (SEQ ID NO:409), TTTTTTTGCATACGTGATGGCTT (SEQ ID NO:411), and TTTTTTTGCATACGTGATGGCTTA (SEQ ID NO:413).

221. The kit of any one of claims 217-220, further comprising: a primer pair comprising the sequences ATGGCTGTGGTTTGTGATGGT (SEQ ID NO:414) and ACACCAGCCATCACGTAAGACA (SEQ ID NO:415).

222. A kit comprising at least five microcarriers, wherein each of said at least five microcarriers comprises:
(i) a probe coupled to the microcarrier, wherein the probe is specific for an RNA mutation in the ALK, ROS, RET, NTRK1, or cMET gene; and
(ii) an identifier corresponding to the probe coupled thereto;
wherein the kit comprises at least one microcarrier comprising a probe specific for an RNA mutation in the ALK gene, at least one microcarrier comprising a probe specific for an RNA mutation in the ROS gene, at least one microcarrier comprising a probe specific for an RNA mutation in the RET gene, at least one microcarrier comprising a probe specific for an RNA mutation in the NTRK1 gene, and at least one microcarrier comprising a probe specific for an RNA mutation in the cMET gene; and
wherein the ALK, ROS, RET, NTRK1, and cMET genes are human genes.

223. The kit of claim 222, wherein each of the mutations in the ALK, ROS, RET, and NTRK1 genes comprises a fusion gene.

224. The kit of claim 223, wherein the mutation in the ALK gene comprises one or more of EML E13:ALK E20, EML E20:ALK E20, and EML E6:ALK E20 EML4-ALK fusion genes.

225. The kit of claim 224, wherein the mutation in the ALK gene comprises EML E13:ALK E20, EML E20:ALK E20, and EML E6:ALK E20 EML4-ALK fusion genes.

226. The kit of claim 225, wherein the probe specific for the mutation in the ALK gene comprises:
(1) a first probe comprising a sequence selected from the group consisting of AAAGGACCTAAAGTGT (SEQ ID NO: 161), CCTAAAGTGTACCGC (SEQ ID NO:163), GGGAAAGGACCTAAAG (SEQ ID NO:165), AGTGTACCGCCGGAA (SEQ ID NO:167), and TACCGCCGGAAGCACC (SEQ ID NO:169);
(2) a second probe comprising a sequence selected from the group consisting of GACTATGAAATATTGTAC (SEQ ID NO:171), GAAATATTGTACTTGTAC (SEQ ID NO:173), TATTGTACTTGTACCGCC (SEQ ID NO:175), TGTACCGCCGGAAGCAC (SEQ ID NO:177), and CCGCCGGAAGCACCAGGA (SEQ ID NO:179);
(3) a third probe comprising a sequence selected from the group consisting of TGTCATCATCAACCAA (SEQ ID NO:181), ATGTCATCATCAACC (SEQ ID NO: 183), GTGTACCGCCGGAAGC (SEQ ID NO:185), TCAACCAAGTGTACCG (SEQ ID NO:187), and TACCGCCGGAAGCACCA (SEQ ID NO:189); and
(4) a fourth probe comprising a sequence selected from the group consisting of CGAAAAAAACAGCCAA (SEQ ID NO:191), TCGCGAAAAAAACAGC (SEQ ID NO:193), GTGTACCGCCGGAAGC (SEQ ID NO:195), TACCGCCGGAAGCACC (SEQ ID NO:197), and ACAGCCAAGTGTACCG (SEQ ID NO:199);
wherein each of the four probes is coupled to a microcarrier with a different identifier.

227. The kit of claim 226, wherein each of the four probes further comprises eight nucleotides at the 5′ end, and wherein the eight nucleotides at the 5′ end are adenine or thymine nucleotides.

228. The kit of claim 227, wherein the probe specific for the mutation in the ALK gene comprises:
(1) a first probe comprising a sequence selected from the group consisting of TTTTTTTTTTAAAGGACCTAAAGTGT (SEQ ID NO: 162), TTTTTTTTTTCCTAAAGTGTACCGC (SEQ ID NO: 164), TTTTTTTTTTGGGAAAGGACCTAAAG (SEQ ID NO:166), TTTTTTTTTTAGTGTACCGCCGGAA (SEQ ID NO:168), and TTTTTTTTTTTACCGCCGGAAGCACC (SEQ ID NO: 170);
(2) a second probe comprising a sequence selected from the group consisting of TTTTTTTTTTTTGACTATGAAATATTGTAC (SEQ ID NO:172), TTTTTTTTTTTTGAAATATTGTACTTGTAC (SEQ ID NO:174), TTTTTTTTTTTTTATTGTACTTGTACCGCC (SEQ ID NO:176), TTTTTTTTTTTTTTGTACCGCCGGAAGCAC (SEQ ID NO:178), and TTTTTTTTTTTTCCGCCGGAAGCACCAGGA (SEQ ID NO:180);
(3) a third probe comprising a sequence selected from the group consisting of TTTTTTTTTTTTTTTGTCATCATCAACCAA (SEQ ID NO:182), TTTTTTTTTTTTTTATGTCATCATCAACC (SEQ ID NO:184), TTTTTTTTTTTTTTGTGTACCGCCGGAAGC (SEQ ID NO:186), TTTTTTTTTTTTTTTCAACCAAGTGTACCG (SEQ ID NO:188), and TTTTTTTTTTTTTTTACCGCCGGAAGCACCA (SEQ ID NO:190); and
(4) a fourth probe comprising a sequence selected from the group consisting of TTTTTTTTTTTTTCGAAAAAAACAGCCAA (SEQ ID NO:192), TTTTTTTTTTTTTTCGCGAAAAAAACAGC (SEQ ID NO:194), TTTTTTTTTTTTTGTGTACCGCCGGAAGC (SEQ ID NO: 196), TTTTTTTTTTTTTTACCGCCGGAAGCACC (SEQ ID NO: 198), and TTTTTTTTTTTTTTACAGCCAAGTGTACCG (SEQ ID NO:200);
wherein each of the four probes is coupled to a microcarrier with a different identifier.

229. The kit of any one of claims 224-228, further comprising: a first primer that is suitable for generating cDNA specific for the mutation in the ALK gene, wherein the first primer comprises the sequence AGTTGGGGTTGTAGTCGGTCAT (SEQ ID NO:363) or GAAGCCTCCCTGGATCTCC (SEQ ID NO:364); and a second primer specific for the mutation in the ALK gene that comprises a sequence selected from the group consisting of TATGGAGCAAAACTACTGTAGAGCC (SEQ ID NO:357), CCAGCTACATCACACACCTTGACT (SEQ ID NO:358), and TAATACCAAAAGTTACCAAAACTGCA (SEQ ID NO:359).

230. The kit of any one of claims 223-229, wherein the mutation in the ROS gene comprises an ROS fusion gene selected from the group consisting of CD74-ROS, and SLC34A2-ROS.

231. The kit of claim 230, wherein the mutation in the ROS gene comprises CD74 E6:ROS E32, CD74 E6:ROS E34, SLC34A2 E4:ROS E32, and SLC34A2 E4:ROS E34 fusion genes.

232. The kit of claim 231, wherein the probe specific for the mutation in the ROS gene comprises:
(1) a first probe comprising a sequence selected from the group consisting of ACTGACGCTCCACCGAAA (SEQ ID NO:201), CCACTGACGCTCCACCGA (SEQ ID NO:203), GCTGGAGTCCCAAATAAAC (SEQ ID NO:205), GGAGTCCCAAATAAACCAG (SEQ ID NO:207), and CACCGAAAGCTGGAGTCCC (SEQ ID NO:209);
(2) a second probe comprising a sequence selected from the group consisting of CCGAAAGATGATTTT (SEQ ID NO:211), GACGCTCCACCGAAA (SEQ ID NO:213), ACTGACGCTCCACCGA (SEQ ID NO:215), GATGATTTTTGGATA (SEQ ID NO:217), and TGATTTTTGGATACCA (SEQ ID NO: 219);
(3) a third probe comprising a sequence selected from the group consisting of AGCGCCTTCCAGCTGGTTGGA (SEQ ID NO:221), CTGGTTGGAGCTGGAGTCCC (SEQ ID NO:223), AGTAGCGCCTTCCAGCTGGTTG (SEQ ID NO:225), GCTGGAGTCCCAAATAAACCA (SEQ ID NO:227), and GGAGTCCCAAATAAACCAGG (SEQ ID NO:229); and
(4) a fourth probe comprising a sequence selected from the group consisting of GCGCCTTCCAGCTGGTTG (SEQ ID NO:231), GTAGCGCCTTCCAGCTGGT (SEQ ID NO:233), TGGTTGGAGATGATTTTT (SEQ ID NO:235), GATGATTTTTGGATACCAG (SEQ ID NO:237), and TGATTTTTGGATACCA (SEQ ID NO:239);
wherein each of the four probes is coupled to a microcarrier with a different identifier.

233. The kit of claim 232, wherein each of the four probes further comprises eight nucleotides at the 5′ end, and wherein the eight nucleotides at the 5′ end are adenine or thymine nucleotides.

234. The kit of claim 233, wherein the probe specific for the mutation in the ROS gene comprises:
(1) a first probe comprising a sequence selected from the group consisting of TTTTTTTTTTTACTGACGCTCCACCGAAA (SEQ ID NO:202), TTTTTTTTTTTCCACTGACGCTCCACCGA (SEQ ID NO:204), TTTTTTTTTTTGCTGGAGTCCCAAATAAAC (SEQ ID NO:206), TTTTTTTTTTTGGAGTCCCAAATAAACCAG (SEQ ID NO:208), and TTTTTTTTTTTCACCGAAAGCTGGAGTCCC (SEQ ID NO:210);
(2) a second probe comprising a sequence selected from the group consisting of TTTTTTTTTTTTCCGAAAGATGATTTT (SEQ ID NO:212), TTTTTTTTTTTTGACGCTCCACCGAAA (SEQ ID NO:214), TTTTTTTTTTTTACTGACGCTCCACCGA (SEQ ID NO:216), TTTTTTTTTTTTGATGATTTTTGGATA (SEQ ID NO:218), and TTTTTTTTTTTTTGATTTTTGGATACCA (SEQ ID NO:220);
(3) a third probe comprising a sequence selected from the group consisting of TTTTTTTTTTTTAGCGCCTTCCAGCTGGTTGGA (SEQ ID NO:222), TTTTTTTTTTTTCTGGTTGGAGCTGGAGTCCC (SEQ ID NO:224), TTTTTTTTTTTTAGTAGCGCCTTCCAGCTGGTTG (SEQ ID NO:226), TTTTTTTTTTTTGCTGGAGTCCCAAATAAACCA (SEQ ID NO:228), and TTTTTTTTTTTTGGAGTCCCAAATAAACCAGG (SEQ ID NO:230); and
(4) a fourth probe comprising a sequence selected from the group consisting of TTTTTTTTTTGCGCCTTCCAGCTGGTTG (SEQ ID NO:232), TTTTTTTTTTGTAGCGCCTTCCAGCTGGT (SEQ ID NO:234), TTTTTTTTTTTGGTTGGAGATGATTTTT (SEQ ID NO:236), TTTTTTTTTTGATGATTTTTGGATACCAG (SEQ ID NO:238), and TTTTTTTTTTTGATTTTTGGATACCA (SEQ ID NO:240);
wherein each of the four probes is coupled to a microcarrier with a different identifier.

235. The kit of any one of claims 230-234, further comprising: a first primer that is suitable for generating cDNA specific for the mutation in the ROS gene, wherein the first primer comprises the sequence AATTCAATACATACTATCAGCTTTCTCCCACTGTATTGAA (SEQ ID NO:21) or AATATTTCTGGTACGAGTGGGATTGTAACAACCAGAAATA (SEQ ID NO: 22); and a second primer specific for the mutation in the ROS gene that comprises the sequence GGAGTGCCATCGCTGTTTGAAATGAGCAGGCACT (SEQ ID NO:19) or TACAGCCCTGGATATTCTTAGTAGCGC (SEQ ID NO:20).

236. The kit of any one of claims 223-235, wherein the mutation in the RET gene comprises a RET fusion gene selected from the group consisting of KIF5B-RET.

237. The kit of claim 235, wherein the mutation in the RET gene comprises KIF5B E15:RET E11, KIF5B E15:RET E12, KIF5B E16:RET E12, KIF5B E22:RET E12, and KIF5B E23:RET E12 fusion genes.

238. The kit of claim 237, wherein the probe specific for the mutation in the RET gene comprises:
(1) a first probe comprising a sequence selected from the group consisting of GTGGGAAATAATGATGTAAA (SEQ ID NO:241), CTGTGGGAAATAATGATGTA (SEQ ID NO:243), GATCCACTGTGCGACGAGCT (SEQ ID NO:245), TGATGTAAAGATCCACTGTG (SEQ ID NO:247), and TCCACTGTGCGACGAGCTGT (SEQ ID NO:249);
(2) a second probe comprising a sequence selected from the group consisting of TGGGAAATAATGATGTAAA (SEQ ID NO:251), CTGTGGGAAATAATGATGTA (SEQ ID NO:253), GGAGGATCCAAAGTGGGAAT (SEQ ID NO:255), GGATCCAAAGTGGGAATT (SEQ ID NO:257), and ATGATGTAAAGGAGGATCC (SEQ ID NO:259);
(3) a third probe comprising a sequence selected from the group consisting of CTTCGTATCTCTCAAGAGGAT (SEQ ID NO:481), GTATCTCTCAAGAGGATCCAA (SEQ ID NO:483), TTCGTATCTCTCAAGAG (SEQ ID NO:485), TCAAGAGGATCCAAA (SEQ ID NO:487), and TCTCTCAAGAGG (SEQ ID NO:489);
(4) a fourth probe comprising a sequence selected from the group consisting of GTTAAAAAGGAGGATCCAA (SEQ ID NO:491), ACAAGAGTTAAAAAGGAGGA (SEQ ID NO:493), AAGAGTTAAAAAGGAGGATC (SEQ ID NO:495), AAAAGGAGGATCCAAAG (SEQ ID NO:497), and AAGGAGGATCCAAAGTG (SEQ ID NO:499); and
(5) a fifth probe comprising a sequence selected from the group consisting of AAACAGGAGGATCCAAA (SEQ ID NO:501), AAGTGCACAAACAGGAGG (SEQ ID NO:503), GTGCACAAACAGGAGGATC (SEQ ID NO:505), CACAAACAGGAGGAT (SEQ ID NO:507), and AACAGGAGGATCCAAA (SEQ ID NO:509);
wherein each of the five probes is coupled to a microcarrier with a different identifier.

239. The kit of claim 238, wherein each of the four probes further comprises eight nucleotides at the 5′ end, and wherein the eight nucleotides at the 5′ end are adenine or thymine nucleotides.

240. The kit of claim 239, wherein the probe specific for the mutation in the RET gene comprises:
(1) a first probe comprising a sequence selected from the group consisting of TTTTTTTTTTGTGGGAAATAATGATGTAAA (SEQ ID NO:242), TTTTTTTTTTCTGTGGGAAATAATGATGTA (SEQ ID NO:244), TTTTTTTTTTGATCCACTGTGCGACGAGCT (SEQ ID NO:246), TTTTTTTTTTTGATGTAAAGATCCACTGTG (SEQ ID NO:248), and TTTTTTTTTTTCCACTGTGCGACGAGCTGT (SEQ ID NO:250);
(2) a second probe comprising a sequence selected from the group consisting of TTTTTTTTTTGGGAAATAATGATGTAAA (SEQ ID NO:252), TTTTTTTTTCTGTGGGAAATAATGATGTA (SEQ ID NO:254), TTTTTTTTTGGAGGATCCAAAGTGGGAAT (SEQ ID NO:256), TTTTTTTTTGGATCCAAAGTGGGAATT (SEQ ID NO:258), and TTTTTTTTTATGATGTAAAGGAGGATCC (SEQ ID NO:260);
(3) a third probe comprising a sequence selected from the group consisting of TTTTTTTTTCTTCGTATCTCTCAAGAGGAT (SEQ ID NO:482), TTTTTTTTTGTATCTCTCAAGAGGATCCAA (SEQ ID NO:484), TTTTTTTTTTTCGTATCTCTCAAGAG (SEQ ID NO:486), TTTTTTTTTTCAAGAGGATCCAAA (SEQ ID NO:488), and TTTTTTTTTTCTCTCAAGAGG (SEQ ID NO:490);
(4) a fourth probe comprising a sequence selected from the group consisting of TTTTTTTTTGTTAAAAAGGAGGATCCAA (SEQ ID NO:492), TTTTTTTTACAAGAGTTAAAAAGGAGGA (SEQ ID NO:494), TTATTATTAAGAGTTAAAAAGGAGGATC (SEQ ID NO:811), TTTTTTTTAAAAGGAGGATCCAAAG (SEQ ID NO:498), and TTTTTTTTAAGGAGGATCCAAAGTG (SEQ ID NO:500); and
(5) a fifth probe comprising a sequence selected from the group consisting of TTTTTTTTAAACAGGAGGATCCAAA (SEQ ID NO:502), TTTTTATTAAGTGCACAAACAGGAGG (SEQ ID NO:504), TATTATTATGTGCACAAACAGGAGGATC (SEQ ID NO:506), TATTTTTTCACAAACAGGAGGAT (SEQ ID NO:508), and TTTTATTTAACAGGAGGATCCAAA (SEQ ID NO:510);
wherein each of the five probes is coupled to a microcarrier with a different identifier.

241. The kit of any one of claims 235-240, further comprising: a first primer that is suitable for generating cDNA specific for the mutation in the RET gene, wherein the first primer comprises the sequence GTGATCGCACAGTAGGACAGCGGCTGCGATC (SEQ ID NO:26) or CTCTAGGAGATATCATTCCAAATTCGCCTTCTCCTAG (SEQ ID NO:27); and a second primer specific for the mutation in the RET gene that comprises a sequence selected from the group consisting of TTTCTGGTGCTATGAGGAAATGACCAACCACCAGA (SEQ ID NO:23), AAGGAGTTAGCAGCATGTCAGC (SEQ ID NO:519), AACTTCAGACTTTACACAACCTGC (SEQ ID NO:520), and ATTGATTCTGATGACACCGGA (SEQ ID NO:521).

242. The kit of any one of claims 223-241, wherein the mutation in the NTRK1 gene comprises a CD74-NTRK1 fusion gene.

243. The kit of claim 242, wherein the mutation in the NTRK1 gene comprises a CD74 E8:NTRK1 E12 fusion gene.

244. The kit of claim 243, wherein the probe specific for the mutation in the NTRK1 gene comprises a sequence selected from the group consisting of CAGGATCTGGGCCCAGACA (SEQ ID NO:261), GATCTGGGCCCAGACACTA (SEQ ID NO:263), CCAGACACTAACAGCACAT (SEQ ID NO:265), GGGCCCAGACACTAACAGC (SEQ ID NO:267), and CTAACAGCACATCTGGAGA (SEQ ID NO:269).

245. The kit of claim 244, wherein the probe specific for the mutation in the NTRK1 gene further comprises eight nucleotides at the 5′ end, and wherein the eight nucleotides at the 5′ end are adenine or thymine nucleotides.

246. The kit of claim 245, wherein the probe specific for the mutation in the NTRK1 gene comprises a sequence selected from the group consisting of TTTTTTTTTTACAGGATCTGGGCCCAGACA (SEQ ID NO:262), TTTTTTTTTTAGATCTGGGCCCAGACACTA (SEQ ID NO:264), TTTTTTTTTTACCAGACACTAACAGCACAT (SEQ ID NO:266), TTTTTTTTTTAGGGCCCAGACACTAACAGC (SEQ ID NO:268), and TTTTTTTTTTACTAACAGCACATCTGGAGA (SEQ ID NO:270).

247. The kit of any one of claims 242-246, further comprising: a first primer that is suitable for generating cDNA specific for the mutation in the NTRK1 gene, wherein the first primer comprises the sequence GGACGAAAATCCAGACCCCAAAAGGTGTTTCGT (SEQ ID NO:32); and a second primer specific for the mutation in the NTRK1 gene that comprises the sequence AGAAGACGTGACAGGAACTGGAGGACCCGTCTT (SEQ ID NO:30).

248. The kit of any one of claims 222-247, wherein the mutation in the cMET gene results in exon skipping.

249. The kit of claim 248, wherein the mutation in the cMET gene results in skipping of exon 14.

250. The kit of claim 249, wherein the probe specific for the mutation in the cMET gene comprises a sequence selected from the group consisting of AGAAAGCAAATTAAAGAT (SEQ ID NO:271), AGCAAATTAAAGATCAG (SEQ ID NO:273), AAATTAAAGATCAGTTTC (SEQ ID NO:275), AGATCAGTTTCCTAATTC (SEQ ID NO:277), and AAGATCAGTTTCCTAATT (SEQ ID NO:279).

251. The kit of claim 250, wherein the probe specific for one or more mutations in the cMET gene further comprises eight nucleotides at the 5′ end, and wherein the eight nucleotides at the 5′ end are adenine or thymine nucleotides.

252. The kit of claim 251, wherein the probe specific for the mutation in the cMET gene comprises a sequence selected from the group consisting of TTTTTTTTTTAGAAAGCAAATTAAAGAT (SEQ ID NO:272), TTTTTTTTTTAGCAAATTAAAGATCAG (SEQ ID NO:274), TTTTTTTTTTAAATTAAAGATCAGTTTC (SEQ ID NO:276), TTTTTTTTTTAGATCAGTTTCCTAATTC (SEQ ID NO:278), and TTTTTTTTTTAAGATCAGTTTCCTAATT (SEQ ID NO:280).

253. The kit of any one of claims 248-252, further comprising: a first primer that is suitable for generating cDNA specific for the mutation in the cMET gene, wherein the first primer comprises the sequence GACAGTATTTTGCAGTAATGGACTGGATATATCAGA (SEQ ID NO:29); and a second primer specific for the mutation in the cMET gene that comprises the sequence GAATTTCACAGGATTGATTGCTGGTGTTGTCTC (SEQ ID NO:28).

254. The kit of any one of claims 149-253, wherein the identifiers of the microcarriers comprise digital barcodes.

255. The kit of claim 254, wherein each of the microcarriers comprises:
(i) a first photopolymer layer;
(ii) a second photopolymer layer; and
(iii) an intermediate layer between the first layer and the second layer, the intermediate layer having an encoded pattern representing the identifier defined thereon, wherein the intermediate layer is partially substantially transmissive and partially substantially opaque to light, representing a code corresponding to the microcarrier, wherein the outermost surface of the microcarrier comprises a photoresist photopolymer, and said photoresist photopolymer is functionalized with the probe specific for the DNA mutation, and wherein said microcarrier has about the same density as water.

256. The kit of any one of claims 149-253, wherein the identifiers of the microcarriers comprise analog codes.

257. The kit of claim 256, wherein each of the microcarriers comprises:
(i) a substantially transparent polymer layer having a first surface and a second surface, the first and the second surfaces being parallel to each other;
(ii) a substantially non-transparent layer that constitutes a two-dimensional shape, wherein the substantially non-transparent layer is affixed to the first surface of the substantially transparent polymer layer and encloses a center portion of the substantially transparent polymer layer, wherein the two-dimensional shape of the substantially non-transparent layer represents an analog code, and wherein the analog code corresponds to the identifier; and
(iii) the probe specific for the mutation, wherein the probe is coupled to at least one of the first surface and the second surface of the substantially transparent polymer layer in at least the center portion of the substantially transparent polymer layer.

258. The kit of claim 257, wherein each of the microcarriers further comprises an orientation indicator for orienting the analog code of the substantially non-transparent polymer layer.

259. The kit of claim 257 or claim 258, wherein the polymer of the substantially transparent polymer layer comprises an epoxy-based polymer.

260. The kit of claim 259, wherein the epoxy-based polymer is SU-8.

261. A kit, comprising:
(a) a plurality of probes, wherein each probe of the plurality is coupled to a microcarrier that has a unique identifier corresponding to the probe coupled thereto, the plurality of probes comprising a first probe comprising the sequence TTTTTTTTTTTTAATAGTTGGAGCT (SEQ ID NO:39); a second probe comprising the sequence TTTTTTTTTTTAGGCGTAGGCAAGA (SEQ ID NO:57); a third probe comprising the sequence TTTTTTTTTTTAAGGAGCTTGTGGC (SEQ ID NO:63); a fourth probe comprising the sequence TTTTTTTCCTGCTCAGTGATTTTA (SEQ ID NO:94); a fifth probe comprising the sequence TTTTTTTTTTTATCTCCTGCTTAG (SEQ ID NO:102); a sixth probe comprising the sequence TTTTTTTTTTTTAATGATGCACGTCA (SEQ ID NO:114); a seventh probe comprising the sequence TTTTTTAATTCTACAGAGAAATCTCGA (SEQ ID NO:82); an eighth probe comprising the sequence TTTTTTTTTTTTTAGTGCTGGCCT (SEQ ID NO:124); a ninth probe comprising the sequence TTTTTTTTTTTACAAAACATCTCCG (SEQ ID NO:131); a tenth probe comprising the sequence TTTTTTTTAATCAAGACATCTC (SEQ ID NO: 141); an eleventh probe comprising the sequence TTTTTTTTCATGAGATGCATGATGA (SEQ ID NO:356); a twelfth probe comprising the sequence TTTTTTTTTTTACAGGAGGCTGCCGA (SEQ ID NO:464); athirteenth probe comprising the sequence TTTTTTTTTTTACAGGAGGGAGCCG (SEQ ID NO:478); a fourteenth probe comprising the sequence TTTTTTTAGATGGCCATCTTG (SEQ ID NO:426); a fifteenth probe comprising the sequence TTTTTTTTTTTGTGATGGCCGG (SEQ ID NO:432); a sixteenth probe comprising the sequence TTTTTTTTGGACAACCCCCATCAC (SEQ ID NO:450); a seventeenth probe comprising the sequence TTTTTTTGCCAGCGTGGACGGTA (SEQ ID NO:460); an eighteenth probe comprising the sequence TTTTTTTTAAATGGGCGGGCCA (SEQ ID NO:160); a nineteenth probe comprising the sequence TTTTTTTTTTTTTTGTAGGGAAGTACA (SEQ ID NO:371); a twentieth probe comprising the sequence TTTTTAAATCAGAATCAGAAGGTGG (SEQ ID NO:396); a twentieth probe comprising the sequence TTTTTAAATCAGAATCAGAAGGTGG (SEQ ID NO:396); a twenty-first probe comprising the sequence TTTTTTTTTTTACGTGATGGCTTACGT (SEQ ID NO:407); a twenty-second probe comprising the sequence TTTTTTTTTTAGTGTACCGCCGGAA (SEQ ID NO:168); a twenty-third probe comprising the sequence TTTTTTTTTTTTGACTATGAAATATTGTAC (SEQ ID NO:172); a twenty-fourth probe comprising the sequence TTTTTTTTTTTTTTTACCGCCGGAAGCACCA (SEQ ID NO:190); a twenty-fifth probe comprising the sequence TTTTTTTTTTTTTGTGTACCGCCGGAAGC (SEQ ID NO:196); a twenty-sixth probe comprising the sequence TTTTTTTTTTTGCTGGAGTCCCAAATAAAC (SEQ ID NO:206); a twenty-seventh probe comprising the sequence TTTTTTTTTTTTGACGCTCCACCGAAA (SEQ ID NO:214); a twenty-eighth probe comprising the sequence TTTTTTTTTTTTGGAGTCCCAAATAAACCAGG (SEQ ID NO:230); a twenty-ninth probe comprising the sequence TTTTTTTTTTGATGATTTTTGGATACCAG (SEQ ID NO:238); a thirtieth probe comprising the sequence TTTTTTTTTTGTGGGAAATAATGATGTAAA (SEQ ID NO:242); a thirty-first probe comprising the sequence TTTTTTTTTCTGTGGGAAATAATGATGTA (SEQ ID NO:254); a thirty-second probe comprising the sequence TTTTTTTTTTCTCTCAAGAGG (SEQ ID NO:490); a thirty-third probe comprising the sequence TTTTTTTTAAGGAGGATCCAAAGTG (SEQ ID NO:500); a thirty-fourth probe comprising the sequence TTTTTTTTAAACAGGAGGATCCAAA (SEQ ID NO:502); a thirty-fifth probe comprising the sequence TTTTTTTTTTACCAGACACTAACAGCACAT (SEQ ID NO:266); a thirty-sixth probe comprising the sequence TTTTTTTTTTAGAAAGCAAATTAAAGAT (SEQ ID NO:272);
(b) a plurality of primer pairs, the plurality of primer pairs comprising a first primer pair comprising the sequences GTACTGGTGGAGTATTTGATAGTG (SEQ ID NO:1) and CGTCAAGGCACTCTTGCCTAC (SEQ ID NO:2); a second primer pair comprising the sequences CAATTTCTACAAGAGATCCTCTCTCT (SEQ ID NO:5) and CTCCATTTTAGCACTTACCTGTGAC (SEQ ID NO:6); a third primer pair comprising the sequences ACCCTAGCCTTAGATAAAACTGAGC (SEQ ID NO:7) and TTTGTTGTCCAGCCACCATGA (SEQ ID NO:8); a fourth primer pair comprising the sequences ATAGCCTCAATTCTTACCATCCACAAAATG (SEQ ID NO:9) and CAGATATATTTCTTCATGAAGACCTCACAGTAA (SEQ ID NO:10); a fifth primer pair comprising the sequences CTTGTGGAGCCTCTTACACCC (SEQ ID NO: 11) and TGCCGAACGCACCGGA (SEQ ID NO:12); a sixth primer pair comprising the sequences GCCAGTTAACGTCTTCCTTCTC (SEQ ID NO: 13) and ATCGAGGATTTCCTTGTTGGCTT (SEQ ID NO:14); a seventh primer pair comprising the sequences CCTCCACCGTGCAGATCATC (SEQ ID NO:15) and TTCCCTGATTACCTTTGCGAT (SEQ ID NO:16); an eighth primer pair comprising the sequences CCACACTGACGTGCCTCT (SEQ ID NO:511) and GCACACGTAGGGGTTGTCCAAGA (SEQ ID NO:512); a ninth primer pair comprising the sequences CCACACTGACGTGCCTCT (SEQ ID NO:513) and GTACACGCTGGCCACGCCG (SEQ ID NO:514); a tenth primer pair comprising the sequences CCACACTGACGTGCCTCT (SEQ ID NO:515) and CAGGCGGCACACGTGAT (SEQ ID NO:516); an eleventh primer pair comprising the sequences CCACACTGACGTGCCTCT (SEQ ID NO:517) and AGGCGGCACACGTGCGGGTTAC (SEQ ID NO:518); a twelfth primer pair comprising the sequences GGAGGACCGTCGCTTGG (SEQ ID NO:17) and TCTTTCTCTTCCGCACCCAG (SEQ ID NO:18); a thirteenth primer pair comprising the sequences GAGGGTCTGACGGGTAGAGTG (SEQ ID NO:380) and TGGCCGCCAGGTCTTGATGTA (SEQ ID NO:381), a fourteenth primer pair comprising the sequences CTTGATGAGCAGCAGCGAAA (SEQ ID NO:397) and CCTTCAGTTCTCCCACCTTCTG (SEQ ID NO:398); a fifteenth primer pair comprising the sequences ATGGCTGTGGTTTGTGATGGT (SEQ ID NO:414) and ACACCAGCCATCACGTAAGACA (SEQ ID NO:415); a sixteenth primer pair comprising the sequences AGTTGGGGTTGTAGTCGGTCAT (SEQ ID NO:363) and TATGGAGCAAAACTACTGTAGAGCC (SEQ ID NO:357); a seventeenth primer pair comprising the sequences AGTTGGGGTTGTAGTCGGTCAT (SEQ ID NO:363) and CCAGCTACATCACACACCTTGACT (SEQ ID NO:358); an eighteenth primer pair comprising the sequences AGTTGGGGTTGTAGTCGGTCAT (SEQ ID NO:363) and TAATACCAAAAGTTACCAAAACTGCA (SEQ ID NO:359); a nineteenth primer pair comprising the sequences GGAGTGCCATCGCTGTTTGAAATGAGCAGGCACT (SEQ ID NO:19); a twentieth primer pair comprising the sequences GGAGTGCCATCGCTGTTTGAAATGAGCAGGCACT (SEQ ID NO:19); a twenty-first primer pair comprising the sequences TACAGCCCTGGATATTCTTAGTAGCGC (SEQ ID NO:20); a twenty-second primer pair comprising the sequences TACAGCCCTGGATATTCTTAGTAGCGC (SEQ ID NO:20); a twenty-third primer pair comprising the sequences GTGATCGCACAGTAGGACAGCGGCTGCGATC (SEQ ID NO:26) and TTTCTGGTGCTATGAGGAAATGACCAACCACCAGA (SEQ ID NO:23); a twenty-fourth primer pair comprising the sequences CTCTAGGAGATATCATTCCAAATTCGCCTTCTCCTAG (SEQ ID NO:27) and TTTCTGGTGCTATGAGGAAATGACCAACCACCAGA (SEQ ID NO:23); a twenty-fifth primer pair comprising the sequences CTCTAGGAGATATCATTCCAAATTCGCCTTCTCCTAG (SEQ ID NO:27) and AAGGAGTTAGCAGCATGTCAGC (SEQ ID NO:519); a twenty-sixth primer pair comprising the sequences CTCTAGGAGATATCATTCCAAATTCGCCTTCTCCTAG (SEQ ID NO:27) and AACTTCAGACTTTACACAACCTGC (SEQ ID NO:520); a twenty-seventh primer pair comprising the sequences CTCTAGGAGATATCATTCCAAATTCGCCTTCTCCTAG (SEQ ID NO:27) and ATTGATTCTGATGACACCGGA (SEQ ID NO:521); a twenty-eighth primer pair comprising the sequences GGACGAAAATCCAGACCCCAAAAGGTGTTTCGT (SEQ ID NO:32) and AGAAGACGTGACAGGAACTGGAGGACCCGTCTT (SEQ ID NO:30); a twenty-ninth primer pair comprising the sequences GACAGTATTTTGCAGTAATGGACTGGATATATCAGA (SEQ ID NO:29) and GAATTTCACAGGATTGATTGCTGGTGTTGTCTC (SEQ ID NO:28); and
(c) a plurality of blocking nucleic acids, the plurality of blocking nucleic acids comprising a first blocking nucleic acid comprising the sequence TTGGAGCTGGTGGCGT(invdT)ₙ, wherein n is 1, 2, or 3 (SEQ ID NO:285); a second blocking nucleic acid comprising the sequence CTGAAATCACTGAGCAGG(invdT)ₙ, wherein n is 1, 2, or 3 (SEQ ID NO:291); a third blocking nucleic acid comprising the sequence CCACCATGATGTGCATCA(invdT)ₙ, wherein n is 1, 2, or 3 (SEQ ID NO:299); a fourth blocking nucleic acid comprising the sequence GAGATTTCACTGTAGC(invdT)ₙ, wherein n is 1, 2, or 3 (SEQ ID NO:301); a fifth blocking nucleic acid comprising the sequence CGCACCGGAGCCCAGCACTTA (invdT)ₙ, wherein n is 1, 2, or 3 (SEQ ID NO:310); a sixth blocking nucleic acid comprising the sequence CGGAGATGTTGCTTCTCT(invdT)ₙ, wherein n is 1, 2, or 3 (SEQ ID NO:312); a seventh blocking nucleic acid comprising the sequence TGCAGCTCATCACGCAGC(invdT)ₙ, wherein n is 1, 2, or 3 (SEQ ID NO:317); an eighth blocking nucleic acid comprising the sequence CCAGCAGTTTGGCCAGCCCT(invdT)ₙ, wherein n is 1, 2, or 3 (SEQ ID NO:322); a ninth blocking nucleic acid comprising the sequence GATGTACTCCCCT (invdT)ₙ, wherein n is 1, 2, or 3 (SEQ ID NO:383); and a tenth blocking nucleic acid comprising the sequence CACCTTCTGCTTCTGGGTAAGA (invdT)ₙ, wherein n is 1, 2, or 3 (SEQ ID NO:403).

262. A kit, comprising:
(a) a plurality of probes, wherein each probe of the plurality is coupled to a microcarrier that has a unique identifier corresponding to the probe coupled thereto, the plurality of probes comprising a first probe comprising the sequence TTTTTTTTTTTTAATGATGGCGTAG (SEQ ID NO:43); a second probe comprising the sequence TTTTTTTTTTTAGTAGTTGGAGCTG (SEQ ID NO:51); a third probe comprising the sequence TTTTTTTTTTTAATTGTGGCGTAGG (SEQ ID NO:65); a fourth probe comprising the sequence TTTTTTTTTAGCTCAGTGATTTTAG (SEQ ID NO:88); a fifth probe comprising the sequence TTTTTTTTTTTTTTTCCTGCTTAGTG (SEQ ID NO:104): a sixth probe comprising the sequence TTTTTTTTTTTTTGATGCACGTC (SEQ ID NO:112); a seventh probe comprising the sequence TTTTTTAATTGAGAAATCTCGATGGAG (SEQ ID NO:78); an eighth probe comprising the sequence TTTTTTAGATCAAAGTGCTGGCCTCCG (SEQ ID NO:120); a ninth probe comprising the sequence TTTTTTTTTTTTTTAAACATCTCCGAAAGCC (SEQ ID NO:135); a tenth probe comprising the sequence TTTTTTTTTTTCAAGACATCTCCGA (SEQ ID NO:145); an eleventh probe comprising the sequence TTTTTTTTTTTGAGATGCATGATGA (SEQ ID NO:352); a twelfth probe comprising the sequence TTTTTTTTTTTACAGGAGGCTGCC (SEQ ID NO:470); a thirteenth probe comprising the sequence TTTTTTTTTTTACCAGGAGGGAGCCG (SEQ ID NO:474); a fourteenth probe comprising the sequence TTTTTTTTTATGGCCATCTTGG (SEQ ID NO:422); a fifteenth probe comprising the sequence TTTTTTTTTTTTTGATGGCCCGCGTG (SEQ ID NO:440); a sixteenth probe comprising the sequence TTTTTTTTTTTTCCCATCACGTGT (SEQ ID NO:448); a seventeenth probe comprising the sequence TTTTTTTTTTTGACGGTAACCCCC (SEQ ID NO:456); an eighteenth probe comprising the sequence TTTTTTTATTTTGGGCGGGCC (SEQ ID NO:152); a nineteenth probe comprising the sequence TTTTTTTTTTTTCTGTAGGGAAGTAC (SEQ ID NO:373); a twentieth probe comprising the sequence TTTTTAAATTTCTTACCCAGAATCA (SEQ ID NO:392); a twenty-first probe comprising the sequence TTTTTAAGCATACGTGATGGCT (SEQ ID NO:409); a twenty-second probe comprising the sequence TTTTTTTTTTGGGAAAGGACCTAAAG (SEQ ID NO:166); a twenty-third probe comprising the sequence TTTTTTTTTTTTCCGCCGGAAGCACCAGGA (SEQ ID NO:180); a twenty-fourth probe comprising the sequence TTTTTTTTTTTTTTGTGTACCGCCGGAAGC (SEQ ID NO:186); a twenty-fifth probe comprising the sequence TTTTTTTTTTTTTCGAAAAAAACAGCCAA (SEQ ID NO:192); a twenty-sixth probe comprising the sequence TTTTTTTTTTTACTGACGCTCCACCGAAA (SEQ ID NO:202); a twenty-seventh probe comprising the sequence TTTTTTTTTTTTGACGCTCCACCGAAA (SEQ ID NO:214); a twenty-eighth probe comprising the sequence TTTTTTTTTTTTAGCGCCTTCCAGCTGGTTGGA (SEQ ID NO:222); a twenty-ninth probe comprising the sequence TTTTTTTTTTTGATTTTTGGATACCA (SEQ ID NO:240); a thirtieth probe comprising the sequence TTTTTTTTTTTCCACTGTGCGACGAGCTGT (SEQ ID NO:250); a thirty-first probe comprising the sequence TTTTTTTTTGGATCCAAAGTGGGAATT (SEQ ID NO:258); a thirty-second probe comprising the sequence TTTTTTTTTTCAAGAGGATCCAAA (SEQ ID NO:488); a thirty-third probe comprising the sequence TTTTTTTTACAAGAGTTAAAAAGGAGGA (SEQ ID NO:494); a thirty-fourth probe comprising the sequence TTTTTATTAAGTGCACAAACAGGAGG (SEQ ID NO:504); a thirty-fifth probe comprising the sequence TTTTTTTTTTACTAACAGCACATCTGGAGA (SEQ ID NO:270); a thirty-sixth probe comprising the sequence TTTTTTTTTTAGATCAGTTTCCTAATTC (SEQ ID NO:278);
(b) a plurality of primer pairs, the plurality of primer pairs comprising a first primer pair comprising the sequences GTACTGGTGGAGTATTTGATAGTG (SEQ ID NO:1) and CGTCAAGGCACTCTTGCCTAC (SEQ ID NO:2); a second primer pair comprising the sequences CAATTTCTACAAGAGATCCTCTCTCT (SEQ ID NO:5) and CTCCATTTTAGCACTTACCTGTGAC (SEQ ID NO:6); a third primer pair comprising the sequences ACCCTAGCCTTAGATAAAACTGAGC (SEQ ID NO:7) and TTTGTTGTCCAGCCACCATGA (SEQ ID NO:8); a fourth primer pair comprising the sequences ATAGCCTCAATTCTTACCATCCACAAAATG (SEQ ID NO:9) and CAGATATATTTCTTCATGAAGACCTCACAGTAA (SEQ ID NO:10); a fifth primer pair comprising the sequences CTTGTGGAGCCTCTTACACCC (SEQ ID NO: 11) and TGCCGAACGCACCGGA (SEQ ID NO:12); a sixth primer pair comprising the sequences GCCAGTTAACGTCTTCCTTCTC (SEQ ID NO:13) and ATCGAGGATTTCCTTGTTGGCTT (SEQ ID NO:14); a seventh primer pair comprising the sequences CCTCCACCGTGCAGATCATC (SEQ ID NO:15) and TTCCCTGATTACCTTTGCGAT (SEQ ID NO:16); an eighth primer pair comprising the sequences CCACACTGACGTGCCTCT (SEQ ID NO:511) and GCACACGTAGGGGTTGTCCAAGA (SEQ ID NO:512); a ninth primer pair comprising the sequences CCACACTGACGTGCCTCT (SEQ ID NO:513) and GTACACGCTGGCCACGCCG (SEQ ID NO:514); a tenth primer pair comprising the sequences CCACACTGACGTGCCTCT (SEQ ID NO:515) and CAGGCGGCACACGTGAT (SEQ ID NO:516); an eleventh primer pair comprising the sequences CCACACTGACGTGCCTCT (SEQ ID NO:517) and AGGCGGCACACGTGCGGGTTAC (SEQ ID NO:518); a twelfth primer pair comprising the sequences GGAGGACCGTCGCTTGG (SEQ ID NO:17) and TCTTTCTCTTCCGCACCCAG (SEQ ID NO:18); a thirteenth primer pair comprising the sequences GAGGGTCTGACGGGTAGAGTG (SEQ ID NO:380) and TGGCCGCCAGGTCTTGATGTA (SEQ ID NO:381); a fourteenth primer pair comprising the sequences CTTGATGAGCAGCAGCGAAA (SEQ ID NO:397) and CCTTCAGTTCTCCCACCTTCTG (SEQ ID NO:398); a fifteenth primer pair comprising the sequences ATGGCTGTGGTTTGTGATGGT (SEQ ID NO:414) and ACACCAGCCATCACGTAAGACA (SEQ ID NO:415); a sixteenth primer pair comprising the sequences AGTTGGGGTTGTAGTCGGTCAT (SEQ ID NO: 363) and TATGGAGCAAAACTACTGTAGAGCC (SEQ ID NO:357); a seventeenth primer pair comprising the sequences AGTTGGGGTTGTAGTCGGTCAT (SEQ ID NO:363) and CCAGCTACATCACACACCTTGACT (SEQ ID NO:358); an eighteenth primer pair comprising the sequences AGTTGGGGTTGTAGTCGGTCAT (SEQ ID NO:363) and TAATACCAAAAGTTACCAAAACTGCA (SEQ ID NO:359); a nineteenth primer pair comprising the sequences GGAGTGCCATCGCTGTTTGAAATGAGCAGGCACT (SEQ ID NO:19); a twentieth primer pair comprising the sequences GGAGTGCCATCGCTGTTTGAAATGAGCAGGCACT (SEQ ID NO:19); a twenty-first primer pair comprising the sequences TACAGCCCTGGATATTCTTAGTAGCGC (SEQ ID NO:20); a twenty-second primer pair comprising the sequences TACAGCCCTGGATATTCTTAGTAGCGC (SEQ ID NO:20); a twenty-third primer pair comprising the sequences GTGATCGCACAGTAGGACAGCGGCTGCGATC (SEQ ID NO:26) and TTTCTGGTGCTATGAGGAAATGACCAACCACCAGA (SEQ ID NO:23); a twenty-fourth primer pair comprising the sequences CTCTAGGAGATATCATTCCAAATTCGCCTTCTCCTAG (SEQ ID NO:27) and TTTCTGGTGCTATGAGGAAATGACCAACCACCAGA (SEQ ID NO:23); a twenty-fifth primer pair comprising the sequences CTCTAGGAGATATCATTCCAAATTCGCCTTCTCCTAG (SEQ ID NO:27) and AAGGAGTTAGCAGCATGTCAGC (SEQ ID NO:519); a twenty-sixth primer pair comprising the sequences CTCTAGGAGATATCATTCCAAATTCGCCTTCTCCTAG (SEQ ID NO:27) and AACTTCAGACTTTACACAACCTGC (SEQ ID NO:520); a twenty-seventh primer pair comprising the sequences CTCTAGGAGATATCATTCCAAATTCGCCTTCTCCTAG (SEQ ID NO:27) and ATTGATTCTGATGACACCGGA (SEQ ID NO:521); a twenty-eighth primer pair comprising the sequences GGACGAAAATCCAGACCCCAAAAGGTGTTTCGT (SEQ ID NO:32) and AGAAGACGTGACAGGAACTGGAGGACCCGTCTT (SEQ ID NO:30); a twenty-ninth primer pair comprising the sequences GACAGTATTTTGCAGTAATGGACTGGATATATCAGA (SEQ ID NO:29) and GAATTTCACAGGATTGATTGCTGGTGTTGTCTC (SEQ ID NO:28); and
(c) a plurality of blocking nucleic acids, the plurality of blocking nucleic acids comprising a first blocking nucleic acid comprising the sequence TTGGAGCTGGTGGCGTA(invdT)ₙ, wherein n is 1, 2, or 3 (SEQ ID NO:282); a second blocking nucleic acid comprising the sequence TCTCTGAAATCACTGAGCAGG(invdT)ₙ, wherein n is 1, 2, or 3 (SEQ ID NO:294); a third blocking nucleic acid comprising the sequence CCACCATGATGTGCAT(invdT)ₙ, wherein n is 1, 2, or 3 (SEQ ID NO:297); a fourth blocking nucleic acid comprising the sequence GAGATTTCACTGTAGC(invdT)ₙ, wherein n is 1, 2, or 3 (SEQ ID NO:305); a fifth blocking nucleic acid comprising the sequence CGCACCGGAGCCCAGCAC (invdT)ₙ, wherein n is 1, 2, or 3 (SEQ ID NO:309); a sixth blocking nucleic acid comprising the sequence GTTGCTTCTCTTAATTCC(invdT)ₙ, wherein n is 1, 2, or 3 (SEQ ID NO:313); a seventh blocking nucleic acid comprising the sequence CTCATCACGCAGC(invdT)ₙ, wherein n is 1, 2, or 3 (SEQ ID NO:320); an eighth blocking nucleic acid comprising the sequence CCAGCAGTTTGGCCAGCCCT(invdT)ₙ, wherein n is 1, 2, or 3 (SEQ ID NO:325); a ninth blocking nucleic acid comprising the sequence; GATGTACTCCCCTACA (invdT)ₙ, wherein n is 1, 2, or 3 (SEQ ID NO:386); and a tenth blocking nucleic acid comprising the sequence CACCTTCTGCTTCTGGG (invdT)ₙ, wherein n is 1, 2, or 3 (SEQ ID NO:401).

263. A kit, comprising:
(a) a plurality of probes, wherein each probe of the plurality is coupled to a microcarrier that has a unique identifier corresponding to the probe coupled thereto, the plurality of probes comprising a first probe comprising the sequence TTTTTTTTTTTATGGAGCTGATGGC (SEQ ID NO:45); a second probe comprising the sequence TTTTTTTTTTTATGGAGCTGTTGGC (SEQ ID NO:53); a third probe comprising the sequence TTTTTTTTTTTAAGGAGCTTGTGGC (SEQ ID NO:63); a fourth probe comprising the sequence TTTTTTTTTTTCTCAGTGATTTTAGA (SEQ ID NO:96); a fifth probe comprising the sequence TTTTTTTTTTTTTCTCCTGCTTAGT (SEQ ID NO:100); a sixth probe comprising the sequence TTTTTTTTTTTTAATGATGCACGTC (SEQ ID NO:116); a seventh probe comprising the sequence TTTTTTAATTTCTAGCTACAGAGAAAT (SEQ ID NO:86); an eighth probe comprising the sequence TTTTTTTTTTCAAAGTGCTGGCCTC (SEQ ID NO:118); a ninth probe comprising the sequence TTTTTTTTTAATCAAAACATCTCCG (SEQ ID NO: 127); a tenth probe comprising the sequence TTTTTTTTAATCAAGACATCTCCGA (SEQ ID NO:137); an eleventh probe comprising the sequence TTTTTTTTTTGAGATGCATGATGAG (SEQ ID NO:353); a twelfth probe comprising the sequence TTTTTTTTTTTACCAGGAGGCTGCC (SEQ ID NO:468); a thirteenth probe comprising the sequence TTTTTTTTTTTACCAGGAGGGAGCC (SEQ ID NO:472); a fourteenth probe comprising the sequence TTTTTTTTTTAGGCCATCTTGGA (SEQ ID NO:424); a fifteenth probe comprising the sequence TTTTTTTTTTTGTGATGGCCGGCGT (SEQ ID NO:436); a sixteenth probe comprising the sequence TTTTTTTTTTTAACCCCCATCACGT (SEQ ID NO:442); a seventeenth probe comprising the sequence TTTTTTTTTTTCGTGGACGGTAACC (SEQ ID NO:454); an eighteenth probe comprising the sequence TTTTTTTAAAAAAGCGGGCCAAACT (SEQ ID NO:156); a nineteenth probe comprising the sequence TTTTTTTTACGTCTGTAGGGAAGTA (SEQ ID NO:379); a twentieth probe comprising the sequence TTTTTAAATTTACCCAGAATCAGAA (SEQ ID NO:388); a twenty-first probe comprising the sequence TTTTTTTTTATACGTGATGTCTTAC (SEQ ID NO:405); a twenty-second probe comprising the sequence TTTTTTTTTTCCTAAAGTGTACCGC (SEQ ID NO:164); a twenty-third probe comprising the sequence TTTTTTTTTTTTTATTGTACTTGTACCGCC (SEQ ID NO:176); a twenty-fourth probe comprising the sequence TTTTTTTTTTTTTTTCAACCAAGTGTACCG (SEQ ID NO:188); a twenty-fifth probe comprising the sequence TTTTTTTTTTTTTTACAGCCAAGTGTACCG (SEQ ID NO:200); a twenty-sixth probe comprising the sequence TTTTTTTTTTTCACCGAAAGCTGGAGTCCC (SEQ ID NO:210); a twenty-seventh probe comprising the sequence TTTTTTTTTTTTCCGAAAGATGATTTT (SEQ ID NO:212); a twenty-eighth probe comprising the sequence TTTTTTTTTTTTCTGGTTGGAGCTGGAGTCCC (SEQ ID NO:224); a twenty-ninth probe comprising the sequence TTTTTTTTTTTGGTTGGAGATGATTTTT (SEQ ID NO:236); a thirtieth probe comprising the sequence TTTTTTTTTTTGATGTAAAGATCCACTGTG (SEQ ID NO:248); a thirty-first probe comprising the sequence TTTTTTTTTATGATGTAAAGGAGGATCC (SEQ ID NO:260); a thirty-second probe comprising the sequence TTTTTTTTTGTATCTCTCAAGAGGATCCAA (SEQ ID NO:484); a thirty-third probe comprising the sequence TTATTATTAAGAGTTAAAAAGGAGGATC (SEQ ID NO:811); a thirty-fourth probe comprising the sequence TATTATTATGTGCACAAACAGGAGGATC (SEQ ID NO:506); a thirty-fifth probe comprising the sequence TTTTTTTTTTAGGGCCCAGACACTAACAGC (SEQ ID NO:268); a thirty-sixth probe comprising the sequence TTTTTTTTTTAAATTAAAGATCAGTTTC (SEQ ID NO:276);
(b) a plurality of primer pairs, the plurality of primer pairs comprising a first primer pair comprising the sequences GTACTGGTGGAGTATTTGATAGTG (SEQ ID NO:1) and CGTCAAGGCACTCTTGCCTAC (SEQ ID NO:2); a second primer pair comprising the sequences CAATTTCTACAAGAGATCCTCTCTCT (SEQ ID NO:5) and CTCCATTTTAGCACTTACCTGTGAC (SEQ ID NO:6); a third primer pair comprising the sequences ACCCTAGCCTTAGATAAAACTGAGC (SEQ ID NO:7) and TTTGTTGTCCAGCCACCATGA (SEQ ID NO:8); a fourth primer pair comprising the sequences ATAGCCTCAATTCTTACCATCCACAAAATG (SEQ ID NO:9) and CAGATATATTTCTTCATGAAGACCTCACAGTAA (SEQ ID NO:10); a fifth primer pair comprising the sequences CTTGTGGAGCCTCTTACACCC (SEQ ID NO:11) and TGCCGAACGCACCGGA (SEQ ID NO:12); a sixth primer pair comprising the sequences GCCAGTTAACGTCTTCCTTCTC (SEQ ID NO:13) and ATCGAGGATTTCCTTGTTGGCTT (SEQ ID NO:14); a seventh primer pair comprising the sequences CCTCCACCGTGCAGATCATC (SEQ ID NO:15) and TTCCCTGATTACCTTTGCGAT (SEQ ID NO:16); an eighth primer pair comprising the sequences CCACACTGACGTGCCTCT (SEQ ID NO:511) and GCACACGTAGGGGTTGTCCAAGA (SEQ ID NO:512); a ninth primer pair comprising the sequences CCACACTGACGTGCCTCT (SEQ ID NO: 513) and GTACACGCTGGCCACGCCG (SEQ ID NO:514); a tenth primer pair comprising the sequences CCACACTGACGTGCCTCT (SEQ ID NO:515) and CAGGCGGCACACGTGAT (SEQ ID NO:516); an eleventh primer pair comprising the sequences CCACACTGACGTGCCTCT (SEQ ID NO:517) and AGGCGGCACACGTGCGGGTTAC (SEQ ID NO:518); a twelfth primer pair comprising the sequences GGAGGACCGTCGCTTGG (SEQ ID NO:17) and TCTTTCTCTTCCGCACCCAG (SEQ ID NO:18); a thirteenth primer pair comprising the sequences GAGGGTCTGACGGGTAGAGTG (SEQ ID NO:380) and TGGCCGCCAGGTCTTGATGTA (SEQ ID NO:381), a fourteenth primer pair comprising the sequences CTTGATGAGCAGCAGCGAAA (SEQ ID NO:397) and CCTTCAGTTCTCCCACCTTCTG (SEQ ID NO:398); a fifteenth primer pair comprising the sequences ATGGCTGTGGTTTGTGATGGT (SEQ ID NO:414) and ACACCAGCCATCACGTAAGACA (SEQ ID NO:415); a sixteenth primer pair comprising the sequences AGTTGGGGTTGTAGTCGGTCAT (SEQ ID NO:363) and TATGGAGCAAAACTACTGTAGAGCC (SEQ ID NO:357); a seventeenth primer pair comprising the sequences AGTTGGGGTTGTAGTCGGTCAT (SEQ ID NO:363) and CCAGCTACATCACACACCTTGACT (SEQ ID NO:358); an eighteenth primer pair comprising the sequences AGTTGGGGTTGTAGTCGGTCAT (SEQ ID NO:363) and TAATACCAAAAGTTACCAAAACTGCA (SEQ ID NO:359); a nineteenth primer pair comprising the sequences GGAGTGCCATCGCTGTTTGAAATGAGCAGGCACT (SEQ ID NO:19); a twentieth primer pair comprising the sequences GGAGTGCCATCGCTGTTTGAAATGAGCAGGCACT (SEQ ID NO:19); a twenty-first primer pair comprising the sequences TACAGCCCTGGATATTCTTAGTAGCGC (SEQ ID NO:20); a twenty-second primer pair comprising the sequences TACAGCCCTGGATATTCTTAGTAGCGC (SEQ ID NO:20); a twenty-third primer pair comprising the sequences GTGATCGCACAGTAGGACAGCGGCTGCGATC (SEQ ID NO:26) and TTTCTGGTGCTATGAGGAAATGACCAACCACCAGA (SEQ ID NO:23); a twenty-fourth primer pair comprising the sequences CTCTAGGAGATATCATTCCAAATTCGCCTTCTCCTAG (SEQ ID NO:27) and TTTCTGGTGCTATGAGGAAATGACCAACCACCAGA (SEQ ID NO:23); a twenty-fifth primer pair comprising the sequences CTCTAGGAGATATCATTCCAAATTCGCCTTCTCCTAG (SEQ ID NO:27) and AAGGAGTTAGCAGCATGTCAGC (SEQ ID NO:519); a twenty-sixth primer pair comprising the sequences CTCTAGGAGATATCATTCCAAATTCGCCTTCTCCTAG (SEQ ID NO:27) and AACTTCAGACTTTACACAACCTGC (SEQ ID NO:520); a twenty-seventh primer pair comprising the sequences CTCTAGGAGATATCATTCCAAATTCGCCTTCTCCTAG (SEQ ID NO:27) and ATTGATTCTGATGACACCGGA (SEQ ID NO:521); a twenty-eighth primer pair comprising the sequences GGACGAAAATCCAGACCCCAAAAGGTGTTTCGT (SEQ ID NO:32) and AGAAGACGTGACAGGAACTGGAGGACCCGTCTT (SEQ ID NO:30); a twenty-ninth primer pair comprising the sequences GACAGTATTTTGCAGTAATGGACTGGATATATCAGA (SEQ ID NO:29) and GAATTTCACAGGATTGATTGCTGGTGTTGTCTC (SEQ ID NO:28); and
(c) a plurality of blocking nucleic acids, the plurality of blocking nucleic acids comprising a first blocking nucleic acid comprising the sequence TACGCCACCAGCT(invdT)ₙ, wherein n is 1, 2, or 3 (SEQ ID NO:281); a second blocking nucleic acid comprising the sequence TCTCTGAAATCACTGAGCAGG(invdT)ₙ, wherein n is 1, 2, or 3 (SEQ ID NO:293); a third blocking nucleic acid comprising the sequence CACCATGATGTGCAT(invdT)ₙ, wherein n is 1, 2, or 3 (SEQ ID NO:296); a fourth blocking nucleic acid comprising the sequence GAGATTTCACTGTAGC(invdT)ₙ, wherein n is 1, 2, or 3 (SEQ ID NO:303); a fifth blocking nucleic acid comprising the sequence GAGCCCAGCAC (invdT)ₙ, wherein n is 1, 2, or 3 (SEQ ID NO:308); a sixth blocking nucleic acid comprising the sequence CGGAGATGTTGCTTCTCTTAATTCC(invdT)ₙ, wherein n is 1, 2, or 3 (SEQ ID NO:311); a seventh blocking nucleic acid comprising the sequence CATCACGCAGCTCATG(invdT)ₙ, wherein n is 1, 2, or 3 (SEQ ID NO:316); an eighth blocking nucleic acid comprising the sequence CCAGCAGTTTGGCCAGCCCT(invdT)ₙ, wherein n is 1, 2, or 3 (SEQ ID NO:321); a ninth blocking nucleic acid comprising the sequence TGTACTCCCCTACA (invdT)ₙ, wherein n is 1, 2, or 3 (SEQ ID NO:382); and a tenth blocking nucleic acid comprising the sequence TCTGCTTCTGGGTAAG (invdT)ₙ, wherein n is 1, 2, or 3 (SEQ ID NO:399).


SEQUENCE LISTING (521 sequences)

SEQ ID NO: 1 - DNA, 24 bp, Artificial Sequence
  Synthetic Construct
gtactggtggagtatttgatagtg

SEQ ID NO: 2 - DNA, 21 bp, Artificial Sequence
  Synthetic Construct
cgtcaaggcactcttgcctac

SEQ ID NO: 3 - DNA, 19 bp, Artificial Sequence
  Synthetic Construct
ccacacccccaggattctt

SEQ ID NO: 4 - DNA, 24 bp, Artificial Sequence
  Synthetic Construct
ttggtctctcatggcactgtactc

SEQ ID NO: 5 - DNA, 26 bp, Artificial Sequence
  Synthetic Construct
caatttctacaagagatcctctctct

SEQ ID NO: 6 - DNA, 25 bp, Artificial Sequence
  Synthetic Construct
ctccattttagcacttacctgtgac

SEQ ID NO: 7 - DNA, 25 bp, Artificial Sequence
  Synthetic Construct
accctagccttagataaaactgagc

SEQ ID NO: 8 - DNA, 21 bp, Artificial Sequence
  Synthetic Construct
tttgttgtccagccaccatga

SEQ ID NO: 9 - DNA, 30 bp, Artificial Sequence
  Synthetic Construct
atagcctcaattcttaccatccacaaaatg

SEQ ID NO: 10 - DNA, 33 bp, Artificial Sequence
  Synthetic Construct
cagatatatttcttcatgaagacctcacagtaa

SEQ ID NO: 11 - DNA, 21 bp, Artificial Sequence
  Synthetic Construct
cttgtggagcctcttacaccc

SEQ ID NO: 12 - DNA, 16 bp, Artificial Sequence
  Synthetic Construct
tgccgaacgcaccgga

SEQ ID NO: 13 - DNA, 22 bp, Artificial Sequence
  Synthetic Construct
gccagttaacgtcttccttctc

SEQ ID NO: 14 - DNA, 23 bp, Artificial Sequence
  Synthetic Construct
atcgaggatttccttgttggctt

SEQ ID NO: 15 - DNA, 20 bp, Artificial Sequence
  Synthetic Construct
cctccaccgtgcagatcatc

SEQ ID NO: 16 - DNA, 21 bp, Artificial Sequence
  Synthetic Construct
ttccctgattacctttgcgat

SEQ ID NO: 17 - DNA, 17 bp, Artificial Sequence
  Synthetic Construct
ggaggaccgtcgcttgg

SEQ ID NO: 18 - DNA, 20 bp, Artificial Sequence
  Synthetic Construct
tctttctcttccgcacccag

SEQ ID NO: 19 - DNA, 34 bp, Artificial Sequence
  Synthetic Construct
ggagtgccatcgctgtttgaaatgagcaggcact

SEQ ID NO: 20 - DNA, 27 bp, Artificial Sequence
  Synthetic Construct
tacagccctggatattcttagtagcgc

SEQ ID NO: 21 - DNA, 40 bp, Artificial Sequence
  Synthetic Construct
aattcaatacatactatcagctttctcccactgtattgaa

SEQ ID NO: 22 - DNA, 40 bp, Artificial Sequence
  Synthetic Construct
aatatttctggtacgagtgggattgtaacaaccagaaata

SEQ ID NO: 23 - DNA, 35 bp, Artificial Sequence
  Synthetic Construct
tttctggtgctatgaggaaatgaccaaccaccaga

SEQ ID NO: 24 - DNA, 39 bp, Artificial Sequence
  Synthetic Construct
cttcagtttgcatgatagagctggagacctacaaactga

SEQ ID NO: 25 - DNA, 37 bp, Artificial Sequence
  Synthetic Construct
tatttcatctgatacgaacctgggactgaagatgaaa

SEQ ID NO: 26 - DNA, 31 bp, Artificial Sequence
  Synthetic Construct
gtgatcgcacagtaggacagcggctgcgatc

SEQ ID NO: 27 - DNA, 37 bp, Artificial Sequence
  Synthetic Construct
ctctaggagatatcattccaaattcgccttctcctag

SEQ ID NO: 28 - DNA, 33 bp, Artificial Sequence
  Synthetic Construct
gaatttcacaggattgattgctggtgttgtctc

SEQ ID NO: 29 - DNA, 36 bp, Artificial Sequence
  Synthetic Construct
gacagtattttgcagtaatggactggatatatcaga

SEQ ID NO: 30 - DNA, 33 bp, Artificial Sequence
  Synthetic Construct
agaagacgtgacaggaactggaggacccgtctt

SEQ ID NO: 31 - DNA, 33 bp, Artificial Sequence
  Synthetic Construct
gagctgctacctacagcaggttgaaggagcagc

SEQ ID NO: 32 - DNA, 33 bp, Artificial Sequence
  Synthetic Construct
ggacgaaaatccagaccccaaaaggtgtttcgt

SEQ ID NO: 33 - DNA, 31 bp, Artificial Sequence
  Synthetic Construct
cattgggtgacatcagggagctgccacccaa

SEQ ID NO: 34 - DNA, 28 bp, Artificial Sequence
  Synthetic Construct
ggaagatataattgacactggcaaaaca

SEQ ID NO: 35 - DNA, 25 bp, Artificial Sequence
  Synthetic Construct
attcattatagtcaagggcatatcc

SEQ ID NO: 36 - DNA, 24 bp, Artificial Sequence
  Synthetic Construct
aagtatgctacacccgtttgatta

SEQ ID NO: 37 - DNA, 20 bp, Artificial Sequence
  Synthetic Construct
cgctctggctaaaagtccat

SEQ ID NO: 38 - DNA, 11 bp, Artificial Sequence
  Synthetic Construct
tagttggagct

SEQ ID NO: 39 - DNA, 25 bp, Artificial Sequence
  Synthetic Construct
ttttttttttttaatagttggagct

SEQ ID NO: 40 - DNA, 11 bp, Artificial Sequence
  Synthetic Construct
tgtggtagttg

SEQ ID NO: 41 - DNA, 25 bp, Artificial Sequence
  Synthetic Construct
ttttttttttttaatgtggtagttg

SEQ ID NO: 42 - DNA, 11 bp, Artificial Sequence
  Synthetic Construct
tgatggcgtag

SEQ ID NO: 43 - DNA, 25 bp, Artificial Sequence
  Synthetic Construct
ttttttttttttaatgatggcgtag

SEQ ID NO: 44 - DNA, 13 bp, Artificial Sequence
  Synthetic Construct
tggagctgatggc

SEQ ID NO: 45 - DNA, 25 bp, Artificial Sequence
  Synthetic Construct
tttttttttttatggagctgatggc

SEQ ID NO: 46 - DNA, 11 bp, Artificial Sequence
  Synthetic Construct
gcgtaggcaag

SEQ ID NO: 47 - DNA, 25 bp, Artificial Sequence
  Synthetic Construct
ttttttttttttaagcgtaggcaag

SEQ ID NO: 48 - DNA, 13 bp, Artificial Sequence
  Synthetic Construct
ctgttggcgtagg

SEQ ID NO: 49 - DNA, 25 bp, Artificial Sequence
  Synthetic Construct
tttttttttttactgttggcgtagg

SEQ ID NO: 50 - DNA, 13 bp, Artificial Sequence
  Synthetic Construct
gtagttggagctg

SEQ ID NO: 51 - DNA, 25 bp, Artificial Sequence
  Synthetic Construct
tttttttttttagtagttggagctg

SEQ ID NO: 52 - DNA, 13 bp, Artificial Sequence
  Synthetic Construct
tggagctgttggc

SEQ ID NO: 53 - DNA, 25 bp, Artificial Sequence
  Synthetic Construct
tttttttttttatggagctgttggc

SEQ ID NO: 54 - DNA, 13 bp, Artificial Sequence
  Synthetic Construct
ttgtggtagttgg

SEQ ID NO: 55 - DNA, 25 bp, Artificial Sequence
  Synthetic Construct
tttttttttttattgtggtagttgg

SEQ ID NO: 56 - DNA, 13 bp, Artificial Sequence
  Synthetic Construct
ggcgtaggcaaga

SEQ ID NO: 57 - DNA, 25 bp, Artificial Sequence
  Synthetic Construct
tttttttttttaggcgtaggcaaga

SEQ ID NO: 58 - DNA, 12 bp, Artificial Sequence
  Synthetic Construct
tagttggagctt

SEQ ID NO: 59 - DNA, 25 bp, Artificial Sequence
  Synthetic Construct
tttttttttttaatagttggagctt

SEQ ID NO: 60 - DNA, 12 bp, Artificial Sequence
  Synthetic Construct
gcgtaggcaaga

SEQ ID NO: 61 - DNA, 25 bp, Artificial Sequence
  Synthetic Construct
tttttttttttaagcgtaggcaaga

SEQ ID NO: 62 - DNA, 12 bp, Artificial Sequence
  Synthetic Construct
ggagcttgtggc

SEQ ID NO: 63 - DNA, 25 bp, Artificial Sequence
  Synthetic Construct
tttttttttttaaggagcttgtggc

SEQ ID NO: 64 - DNA, 12 bp, Artificial Sequence
  Synthetic Construct
ttgtggcgtagg

SEQ ID NO: 65 - DNA, 25 bp, Artificial Sequence
  Synthetic Construct
tttttttttttaattgtggcgtagg

SEQ ID NO: 66 - DNA, 12 bp, Artificial Sequence
  Synthetic Construct
tgtggtagttgg

SEQ ID NO: 67 - DNA, 25 bp, Artificial Sequence
  Synthetic Construct
tttttttttttaatgtggtagttgg

SEQ ID NO: 68 - DNA, 16 bp, Artificial Sequence
  Synthetic Construct
gacatactggatacag

SEQ ID NO: 69 - DNA, 27 bp, Artificial Sequence
  Synthetic Construct
tttttttttttgacatactggatacag

SEQ ID NO: 70 - DNA, 16 bp, Artificial Sequence
  Synthetic Construct
actggatacagctgga

SEQ ID NO: 71 - DNA, 27 bp, Artificial Sequence
  Synthetic Construct
tttttttttttactggatacagctgga

SEQ ID NO: 72 - DNA, 16 bp, Artificial Sequence
  Synthetic Construct
actagaagagtacagt

SEQ ID NO: 73 - DNA, 27 bp, Artificial Sequence
  Synthetic Construct
tttttttttttactagaagagtacagt

SEQ ID NO: 74 - DNA, 16 bp, Artificial Sequence
  Synthetic Construct
atacagctggactaga

SEQ ID NO: 75 - DNA, 27 bp, Artificial Sequence
  Synthetic Construct
tttttttttttatacagctggactaga

SEQ ID NO: 76 - DNA, 16 bp, Artificial Sequence
  Synthetic Construct
gctggactagaagagt

SEQ ID NO: 77 - DNA, 27 bp, Artificial Sequence
  Synthetic Construct
tttttttttttgctggactagaagagt

SEQ ID NO: 78 - DNA, 27 bp, Artificial Sequence
  Synthetic Construct
ttttttaattgagaaatctcgatggag

SEQ ID NO: 79 - DNA, 17 bp, Artificial Sequence
  Synthetic Construct
tttggtctagctacaga

SEQ ID NO: 80 - DNA, 27 bp, Artificial Sequence
  Synthetic Construct
ttttttaatttttggtctagctacaga

SEQ ID NO: 81 - DNA, 17 bp, Artificial Sequence
  Synthetic Construct
ctacagagaaatctcga

SEQ ID NO: 82 - DNA, 27 bp, Artificial Sequence
  Synthetic Construct
ttttttaattctacagagaaatctcga

SEQ ID NO: 83 - DNA, 17 bp, Artificial Sequence
  Synthetic Construct
gtgattttggtctagct

SEQ ID NO: 84 - DNA, 27 bp, Artificial Sequence
  Synthetic Construct
ttttttaattgtgattttggtctagct

SEQ ID NO: 85 - DNA, 17 bp, Artificial Sequence
  Synthetic Construct
tctagctacagagaaat

SEQ ID NO: 86 - DNA, 27 bp, Artificial Sequence
  Synthetic Construct
ttttttaatttctagctacagagaaat

SEQ ID NO: 87 - DNA, 15 bp, Artificial Sequence
  Synthetic Construct
gctcagtgattttag

SEQ ID NO: 88 - DNA, 25 bp, Artificial Sequence
  Synthetic Construct
tttttttttagctcagtgattttag

SEQ ID NO: 89 - DNA, 14 bp, Artificial Sequence
  Synthetic Construct
tgctcagtgatttt

SEQ ID NO: 90 - DNA, 23 bp, Artificial Sequence
  Synthetic Construct
ttttttttttgctcagtgatttt

SEQ ID NO: 91 - DNA, 15 bp, Artificial Sequence
  Synthetic Construct
gctcagtgattttag

SEQ ID NO: 92 - DNA, 25 bp, Artificial Sequence
  Synthetic Construct
tttttttttagctcagtgattttag

SEQ ID NO: 93 - DNA, 17 bp, Artificial Sequence
  Synthetic Construct
cctgctcagtgatttta

SEQ ID NO: 94 - DNA, 24 bp, Artificial Sequence
  Synthetic Construct
tttttttcctgctcagtgatttta

SEQ ID NO: 95 - DNA, 15 bp, Artificial Sequence
  Synthetic Construct
ctcagtgattttaga

SEQ ID NO: 96 - DNA, 26 bp, Artificial Sequence
  Synthetic Construct
tttttttttttctcagtgattttaga

SEQ ID NO: 97 - DNA, 12 bp, Artificial Sequence
  Synthetic Construct
ttctcctgctta

SEQ ID NO: 98 - DNA, 23 bp, Artificial Sequence
  Synthetic Construct
tttttttttttttctcctgctta

SEQ ID NO: 99 - DNA, 12 bp, Artificial Sequence
  Synthetic Construct
ctcctgcttagt

SEQ ID NO: 100 - DNA, 25 bp, Artificial Sequence
  Synthetic Construct
tttttttttttttctcctgcttagt

SEQ ID NO: 101 - DNA, 12 bp, Artificial Sequence
  Synthetic Construct
tctcctgcttag

SEQ ID NO: 102 - DNA, 24 bp, Artificial Sequence
  Synthetic Construct
tttttttttttatctcctgcttag

SEQ ID NO: 103 - DNA, 12 bp, Artificial Sequence
  Synthetic Construct
tcctgcttagtg

SEQ ID NO: 104 - DNA, 26 bp, Artificial Sequence
  Synthetic Construct
tttttttttttttttcctgcttagtg

SEQ ID NO: 105 - DNA, 14 bp, Artificial Sequence
  Synthetic Construct
ctcctgcttagtga

SEQ ID NO: 106 - DNA, 27 bp, Artificial Sequence
  Synthetic Construct
tttttttttttttctcctgcttagtga

SEQ ID NO: 107 - DNA, 13 bp, Artificial Sequence
  Synthetic Construct
gatgcacgtcatg

SEQ ID NO: 108 - DNA, 28 bp, Artificial Sequence
  Synthetic Construct
tttttttttttttttgatgcacgtcatg

SEQ ID NO: 109 - DNA, 13 bp, Artificial Sequence
  Synthetic Construct
tgaatgatgcacg

SEQ ID NO: 110 - DNA, 23 bp, Artificial Sequence
  Synthetic Construct
tttttttttttgaatgatgcacg

SEQ ID NO: 111 - DNA, 11 bp, Artificial Sequence
  Synthetic Construct
tgatgcacgtc

SEQ ID NO: 112 - DNA, 23 bp, Artificial Sequence
  Synthetic Construct
tttttttttttttgatgcacgtc

SEQ ID NO: 113 - DNA, 14 bp, Artificial Sequence
  Synthetic Construct
aatgatgcacgtca

SEQ ID NO: 114 - DNA, 26 bp, Artificial Sequence
  Synthetic Construct
ttttttttttttaatgatgcacgtca

SEQ ID NO: 115 - DNA, 13 bp, Artificial Sequence
  Synthetic Construct
aatgatgcacgtc

SEQ ID NO: 116 - DNA, 25 bp, Artificial Sequence
  Synthetic Construct
ttttttttttttaatgatgcacgtc

SEQ ID NO: 117 - DNA, 16 bp, Artificial Sequence
  Synthetic Construct
tcaaagtgctggcctc

SEQ ID NO: 118 - DNA, 25 bp, Artificial Sequence
  Synthetic Construct
ttttttttttcaaagtgctggcctc

SEQ ID NO: 119 - DNA, 21 bp, Artificial Sequence
  Synthetic Construct
agatcaaagtgctggcctccg

SEQ ID NO: 120 - DNA, 27 bp, Artificial Sequence
  Synthetic Construct
ttttttagatcaaagtgctggcctccg

SEQ ID NO: 121 - DNA, 13 bp, Artificial Sequence
  Synthetic Construct
aaagtgctggcct

SEQ ID NO: 122 - DNA, 24 bp, Artificial Sequence
  Synthetic Construct
tttttttttttaaagtgctggcct

SEQ ID NO: 123 - DNA, 11 bp, Artificial Sequence
  Synthetic Construct
agtgctggcct

SEQ ID NO: 124 - DNA, 24 bp, Artificial Sequence
  Synthetic Construct
tttttttttttttagtgctggcct

SEQ ID NO: 125 - DNA, 13 bp, Artificial Sequence
  Synthetic Construct
aagtgctggcctc

SEQ ID NO: 126 - DNA, 25 bp, Artificial Sequence
  Synthetic Construct
ttttttttttttaagtgctggcctc

SEQ ID NO: 127 - DNA, 25 bp, Artificial Sequence
  Synthetic Construct
tttttttttaatcaaaacatctccg

SEQ ID NO: 128 - DNA, 20 bp, Artificial Sequence
  Synthetic Construct
aatcaaaacatctccgaaag

SEQ ID NO: 129 - DNA, 29 bp, Artificial Sequence
  Synthetic Construct
tttttttttaatcaaaacatctccgaaag

SEQ ID NO: 130 - DNA, 13 bp, Artificial Sequence
  Synthetic Construct
caaaacatctccg

SEQ ID NO: 131 - DNA, 25 bp, Artificial Sequence
  Synthetic Construct
tttttttttttacaaaacatctccg

SEQ ID NO: 132 - DNA, 10 bp, Artificial Sequence
  Synthetic Construct
aacatctccg

SEQ ID NO: 133 - DNA, 25 bp, Artificial Sequence
  Synthetic Construct
tttttttttttttttaacatctccg

SEQ ID NO: 134 - DNA, 17 bp, Artificial Sequence
  Synthetic Construct
aaacatctccgaaagcc

SEQ ID NO: 135 - DNA, 31 bp, Artificial Sequence
  Synthetic Construct
ttttttttttttttaaacatctccgaaagcc

SEQ ID NO: 136 - DNA, 17 bp, Artificial Sequence
  Synthetic Construct
aatcaagacatctccga

SEQ ID NO: 137 - DNA, 25 bp, Artificial Sequence
  Synthetic Construct
ttttttttaatcaagacatctccga

SEQ ID NO: 138 - DNA, 19 bp, Artificial Sequence
  Synthetic Construct
gcaatcaagacatctccga

SEQ ID NO: 139 - DNA, 25 bp, Artificial Sequence
  Synthetic Construct
ttttttgcaatcaagacatctccga

SEQ ID NO: 140 - DNA, 14 bp, Artificial Sequence
  Synthetic Construct
aatcaagacatctc

SEQ ID NO: 141 - DNA, 22 bp, Artificial Sequence
  Synthetic Construct
ttttttttaatcaagacatctc

SEQ ID NO: 142 - DNA, 21 bp, Artificial Sequence
  Synthetic Construct
aatcaagacatctccgaaagc

SEQ ID NO: 143 - DNA, 29 bp, Artificial Sequence
  Synthetic Construct
ttttttttaatcaagacatctccgaaagc

SEQ ID NO: 144 - DNA, 14 bp, Artificial Sequence
  Synthetic Construct
caagacatctccga

SEQ ID NO: 145 - DNA, 25 bp, Artificial Sequence
  Synthetic Construct
tttttttttttcaagacatctccga

SEQ ID NO: 146 - DNA, 14 bp, Artificial Sequence
  Synthetic Construct
gagatgcatgatga

SEQ ID NO: 147 - DNA, 16 bp, Artificial Sequence
  Synthetic Construct
tgagatgcatgatgag

SEQ ID NO: 148 - DNA, 17 bp, Artificial Sequence
  Synthetic Construct
atgagatgcatgatgag

SEQ ID NO: 149 - DNA, 15 bp, Artificial Sequence
  Synthetic Construct
tgagctgcatgatga

SEQ ID NO: 150 - DNA, 17 bp, Artificial Sequence
  Synthetic Construct
catgagatgcatgatga

SEQ ID NO: 151 - DNA, 14 bp, Artificial Sequence
  Synthetic Construct
attttgggcgggcc

SEQ ID NO: 152 - DNA, 21 bp, Artificial Sequence
  Synthetic Construct
tttttttattttgggcgggcc

SEQ ID NO: 153 - DNA, 14 bp, Artificial Sequence
  Synthetic Construct
ttgggcgggccaaa

SEQ ID NO: 154 - DNA, 24 bp, Artificial Sequence
  Synthetic Construct
ttttttttaattgggcgggccaaa

SEQ ID NO: 155 - DNA, 12 bp, Artificial Sequence
  Synthetic Construct
gcgggccaaact

SEQ ID NO: 156 - DNA, 25 bp, Artificial Sequence
  Synthetic Construct
tttttttaaaaaagcgggccaaact

SEQ ID NO: 157 - DNA, 14 bp, Artificial Sequence
  Synthetic Construct
gggcgggccaaact

SEQ ID NO: 158 - DNA, 26 bp, Artificial Sequence
  Synthetic Construct
ttttttttaaaagggcgggccaaact

SEQ ID NO: 159 - DNA, 11 bp, Artificial Sequence
  Synthetic Construct
tgggcgggcca

SEQ ID NO: 160 - DNA, 22 bp, Artificial Sequence
  Synthetic Construct
ttttttttaaatgggcgggcca

SEQ ID NO: 161 - DNA, 16 bp, Artificial Sequence
  Synthetic Construct
aaaggacctaaagtgt

SEQ ID NO: 162 - DNA, 26 bp, Artificial Sequence
  Synthetic Construct
ttttttttttaaaggacctaaagtgt

SEQ ID NO: 163 - DNA, 15 bp, Artificial Sequence
  Synthetic Construct
cctaaagtgtaccgc

SEQ ID NO: 164 - DNA, 25 bp, Artificial Sequence
  Synthetic Construct
ttttttttttcctaaagtgtaccgc

SEQ ID NO: 165 - DNA, 16 bp, Artificial Sequence
  Synthetic Construct
gggaaaggacctaaag

SEQ ID NO: 166 - DNA, 26 bp, Artificial Sequence
  Synthetic Construct
ttttttttttgggaaaggacctaaag

SEQ ID NO: 167 - DNA, 15 bp, Artificial Sequence
  Synthetic Construct
agtgtaccgccggaa

SEQ ID NO: 168 - DNA, 25 bp, Artificial Sequence
  Synthetic Construct
ttttttttttagtgtaccgccggaa

SEQ ID NO: 169 - DNA, 16 bp, Artificial Sequence
  Synthetic Construct
taccgccggaagcacc

SEQ ID NO: 170 - DNA, 26 bp, Artificial Sequence
  Synthetic Construct
tttttttttttaccgccggaagcacc

SEQ ID NO: 171 - DNA, 18 bp, Artificial Sequence
  Synthetic Construct
gactatgaaatattgtac

SEQ ID NO: 172 - DNA, 30 bp, Artificial Sequence
  Synthetic Construct
ttttttttttttgactatgaaatattgtac

SEQ ID NO: 173 - DNA, 18 bp, Artificial Sequence
  Synthetic Construct
gaaatattgtacttgtac

SEQ ID NO: 174 - DNA, 30 bp, Artificial Sequence
  Synthetic Construct
ttttttttttttgaaatattgtacttgtac

SEQ ID NO: 175 - DNA, 18 bp, Artificial Sequence
  Synthetic Construct
tattgtacttgtaccgcc

SEQ ID NO: 176 - DNA, 30 bp, Artificial Sequence
  Synthetic Construct
tttttttttttttattgtacttgtaccgcc

SEQ ID NO: 177 - DNA, 17 bp, Artificial Sequence
  Synthetic Construct
tgtaccgccggaagcac

SEQ ID NO: 178 - DNA, 30 bp, Artificial Sequence
  Synthetic Construct
ttttttttttttttgtaccgccggaagcac

SEQ ID NO: 179 - DNA, 18 bp, Artificial Sequence
  Synthetic Construct
ccgccggaagcaccagga

SEQ ID NO: 180 - DNA, 30 bp, Artificial Sequence
  Synthetic Construct
ttttttttttttccgccggaagcaccagga

SEQ ID NO: 181 - DNA, 16 bp, Artificial Sequence
  Synthetic Construct
tgtcatcatcaaccaa

SEQ ID NO: 182 - DNA, 30 bp, Artificial Sequence
  Synthetic Construct
tttttttttttttttgtcatcatcaaccaa

SEQ ID NO: 183 - DNA, 15 bp, Artificial Sequence
  Synthetic Construct
atgtcatcatcaacc

SEQ ID NO: 184 - DNA, 29 bp, Artificial Sequence
  Synthetic Construct
ttttttttttttttatgtcatcatcaacc

SEQ ID NO: 185 - DNA, 16 bp, Artificial Sequence
  Synthetic Construct
gtgtaccgccggaagc

SEQ ID NO: 186 - DNA, 30 bp, Artificial Sequence
  Synthetic Construct
ttttttttttttttgtgtaccgccggaagc

SEQ ID NO: 187 - DNA, 16 bp, Artificial Sequence
  Synthetic Construct
tcaaccaagtgtaccg

SEQ ID NO: 188 - DNA, 30 bp, Artificial Sequence
  Synthetic Construct
tttttttttttttttcaaccaagtgtaccg

SEQ ID NO: 189 - DNA, 17 bp, Artificial Sequence
  Synthetic Construct
taccgccggaagcacca

SEQ ID NO: 190 - DNA, 31 bp, Artificial Sequence
  Synthetic Construct
tttttttttttttttaccgccggaagcacca

SEQ ID NO: 191 - DNA, 16 bp, Artificial Sequence
  Synthetic Construct
cgaaaaaaacagccaa

SEQ ID NO: 192 - DNA, 29 bp, Artificial Sequence
  Synthetic Construct
tttttttttttttcgaaaaaaacagccaa

SEQ ID NO: 193 - DNA, 16 bp, Artificial Sequence
  Synthetic Construct
tcgcgaaaaaaacagc

SEQ ID NO: 194 - DNA, 29 bp, Artificial Sequence
  Synthetic Construct
ttttttttttttttcgcgaaaaaaacagc

SEQ ID NO: 195 - DNA, 16 bp, Artificial Sequence
  Synthetic Construct
gtgtaccgccggaagc

SEQ ID NO: 196 - DNA, 29 bp, Artificial Sequence
  Synthetic Construct
tttttttttttttgtgtaccgccggaagc

SEQ ID NO: 197 - DNA, 16 bp, Artificial Sequence
  Synthetic Construct
taccgccggaagcacc

SEQ ID NO: 198 - DNA, 29 bp, Artificial Sequence
  Synthetic Construct
ttttttttttttttaccgccggaagcacc

SEQ ID NO: 199 - DNA, 16 bp, Artificial Sequence
  Synthetic Construct
acagccaagtgtaccg

SEQ ID NO: 200 - DNA, 30 bp, Artificial Sequence
  Synthetic Construct
ttttttttttttttacagccaagtgtaccg

SEQ ID NO: 201 - DNA, 18 bp, Artificial Sequence
  Synthetic Construct
actgacgctccaccgaaa

SEQ ID NO: 202 - DNA, 29 bp, Artificial Sequence
  Synthetic Construct
tttttttttttactgacgctccaccgaaa

SEQ ID NO: 203 - DNA, 18 bp, Artificial Sequence
  Synthetic Construct
ccactgacgctccaccga

SEQ ID NO: 204 - DNA, 29 bp, Artificial Sequence
  Synthetic Construct
tttttttttttccactgacgctccaccga

SEQ ID NO: 205 - DNA, 19 bp, Artificial Sequence
  Synthetic Construct
gctggagtcccaaataaac

SEQ ID NO: 206 - DNA, 30 bp, Artificial Sequence
  Synthetic Construct
tttttttttttgctggagtcccaaataaac

SEQ ID NO: 207 - DNA, 19 bp, Artificial Sequence
  Synthetic Construct
ggagtcccaaataaaccag

SEQ ID NO: 208 - DNA, 30 bp, Artificial Sequence
  Synthetic Construct
tttttttttttggagtcccaaataaaccag

SEQ ID NO: 209 - DNA, 19 bp, Artificial Sequence
  Synthetic Construct
caccgaaagctggagtccc

SEQ ID NO: 210 - DNA, 30 bp, Artificial Sequence
  Synthetic Construct
tttttttttttcaccgaaagctggagtccc

SEQ ID NO: 211 - DNA, 15 bp, Artificial Sequence
  Synthetic Construct
ccgaaagatgatttt

SEQ ID NO: 212 - DNA, 27 bp, Artificial Sequence
  Synthetic Construct
ttttttttttttccgaaagatgatttt

SEQ ID NO: 213 - DNA, 15 bp, Artificial Sequence
  Synthetic Construct
gacgctccaccgaaa

SEQ ID NO: 214 - DNA, 27 bp, Artificial Sequence
  Synthetic Construct
ttttttttttttgacgctccaccgaaa

SEQ ID NO: 215 - DNA, 16 bp, Artificial Sequence
  Synthetic Construct
actgacgctccaccga

SEQ ID NO: 216 - DNA, 28 bp, Artificial Sequence
  Synthetic Construct
ttttttttttttactgacgctccaccga

SEQ ID NO: 217 - DNA, 15 bp, Artificial Sequence
  Synthetic Construct
gatgatttttggata

SEQ ID NO: 218 - DNA, 27 bp, Artificial Sequence
  Synthetic Construct
ttttttttttttgatgatttttggata

SEQ ID NO: 219 - DNA, 16 bp, Artificial Sequence
  Synthetic Construct
tgatttttggatacca

SEQ ID NO: 220 - DNA, 28 bp, Artificial Sequence
  Synthetic Construct
tttttttttttttgatttttggatacca

SEQ ID NO: 221 - DNA, 21 bp, Artificial Sequence
  Synthetic Construct
agcgccttccagctggttgga

SEQ ID NO: 222 - DNA, 33 bp, Artificial Sequence
  Synthetic Construct
ttttttttttttagcgccttccagctggttgga

SEQ ID NO: 223 - DNA, 20 bp, Artificial Sequence
  Synthetic Construct
ctggttggagctggagtccc

SEQ ID NO: 224 - DNA, 32 bp, Artificial Sequence
  Synthetic Construct
ttttttttttttctggttggagctggagtccc

SEQ ID NO: 225 - DNA, 22 bp, Artificial Sequence
  Synthetic Construct
agtagcgccttccagctggttg

SEQ ID NO: 226 - DNA, 34 bp, Artificial Sequence
  Synthetic Construct
ttttttttttttagtagcgccttccagctggttg

SEQ ID NO: 227 - DNA, 21 bp, Artificial Sequence
  Synthetic Construct
gctggagtcccaaataaacca

SEQ ID NO: 228 - DNA, 33 bp, Artificial Sequence
  Synthetic Construct
ttttttttttttgctggagtcccaaataaacca

SEQ ID NO: 229 - DNA, 20 bp, Artificial Sequence
  Synthetic Construct
ggagtcccaaataaaccagg

SEQ ID NO: 230 - DNA, 32 bp, Artificial Sequence
  Synthetic Construct
ttttttttttttggagtcccaaataaaccagg

SEQ ID NO: 231 - DNA, 18 bp, Artificial Sequence
  Synthetic Construct
gcgccttccagctggttg

SEQ ID NO: 232 - DNA, 28 bp, Artificial Sequence
  Synthetic Construct
ttttttttttgcgccttccagctggttg

SEQ ID NO: 233 - DNA, 19 bp, Artificial Sequence
  Synthetic Construct
gtagcgccttccagctggt

SEQ ID NO: 234 - DNA, 29 bp, Artificial Sequence
  Synthetic Construct
ttttttttttgtagcgccttccagctggt

SEQ ID NO: 235 - DNA, 18 bp, Artificial Sequence
  Synthetic Construct
tggttggagatgattttt

SEQ ID NO: 236 - DNA, 28 bp, Artificial Sequence
  Synthetic Construct
tttttttttttggttggagatgattttt

SEQ ID NO: 237 - DNA, 19 bp, Artificial Sequence
  Synthetic Construct
gatgatttttggataccag

SEQ ID NO: 238 - DNA, 29 bp, Artificial Sequence
  Synthetic Construct
ttttttttttgatgatttttggataccag

SEQ ID NO: 239 - DNA, 16 bp, Artificial Sequence
  Synthetic Construct
tgatttttggatacca

SEQ ID NO: 240 - DNA, 26 bp, Artificial Sequence
  Synthetic Construct
tttttttttttgatttttggatacca

SEQ ID NO: 241 - DNA, 20 bp, Artificial Sequence
  Synthetic Construct
gtgggaaataatgatgtaaa

SEQ ID NO: 242 - DNA, 30 bp, Artificial Sequence
  Synthetic Construct
ttttttttttgtgggaaataatgatgtaaa

SEQ ID NO: 243 - DNA, 20 bp, Artificial Sequence
  Synthetic Construct
ctgtgggaaataatgatgta

SEQ ID NO: 244 - DNA, 30 bp, Artificial Sequence
  Synthetic Construct
ttttttttttctgtgggaaataatgatgta

SEQ ID NO: 245 - DNA, 20 bp, Artificial Sequence
  Synthetic Construct
gatccactgtgcgacgagct

SEQ ID NO: 246 - DNA, 30 bp, Artificial Sequence
  Synthetic Construct
ttttttttttgatccactgtgcgacgagct

SEQ ID NO: 247 - DNA, 20 bp, Artificial Sequence
  Synthetic Construct
tgatgtaaagatccactgtg

SEQ ID NO: 248 - DNA, 30 bp, Artificial Sequence
  Synthetic Construct
tttttttttttgatgtaaagatccactgtg

SEQ ID NO: 249 - DNA, 20 bp, Artificial Sequence
  Synthetic Construct
tccactgtgcgacgagctgt

SEQ ID NO: 250 - DNA, 30 bp, Artificial Sequence
  Synthetic Construct
tttttttttttccactgtgcgacgagctgt

SEQ ID NO: 251 - DNA, 19 bp, Artificial Sequence
  Synthetic Construct
tgggaaataatgatgtaaa

SEQ ID NO: 252 - DNA, 28 bp, Artificial Sequence
  Synthetic Construct
ttttttttttgggaaataatgatgtaaa

SEQ ID NO: 253 - DNA, 20 bp, Artificial Sequence
  Synthetic Construct
ctgtgggaaataatgatgta

SEQ ID NO: 254 - DNA, 29 bp, Artificial Sequence
  Synthetic Construct
tttttttttctgtgggaaataatgatgta

SEQ ID NO: 255 - DNA, 20 bp, Artificial Sequence
  Synthetic Construct
ggaggatccaaagtgggaat

SEQ ID NO: 256 - DNA, 29 bp, Artificial Sequence
  Synthetic Construct
tttttttttggaggatccaaagtgggaat

SEQ ID NO: 257 - DNA, 18 bp, Artificial Sequence
  Synthetic Construct
ggatccaaagtgggaatt

SEQ ID NO: 258 - DNA, 27 bp, Artificial Sequence
  Synthetic Construct
tttttttttggatccaaagtgggaatt

SEQ ID NO: 259 - DNA, 19 bp, Artificial Sequence
  Synthetic Construct
atgatgtaaaggaggatcc

SEQ ID NO: 260 - DNA, 28 bp, Artificial Sequence
  Synthetic Construct
tttttttttatgatgtaaaggaggatcc

SEQ ID NO: 261 - DNA, 19 bp, Artificial Sequence
  Synthetic Construct
caggatctgggcccagaca

SEQ ID NO: 262 - DNA, 30 bp, Artificial Sequence
  Synthetic Construct
ttttttttttacaggatctgggcccagaca

SEQ ID NO: 263 - DNA, 19 bp, Artificial Sequence
  Synthetic Construct
gatctgggcccagacacta

SEQ ID NO: 264 - DNA, 30 bp, Artificial Sequence
  Synthetic Construct
ttttttttttagatctgggcccagacacta

SEQ ID NO: 265 - DNA, 19 bp, Artificial Sequence
  Synthetic Construct
ccagacactaacagcacat

SEQ ID NO: 266 - DNA, 30 bp, Artificial Sequence
  Synthetic Construct
ttttttttttaccagacactaacagcacat

SEQ ID NO: 267 - DNA, 19 bp, Artificial Sequence
  Synthetic Construct
gggcccagacactaacagc

SEQ ID NO: 268 - DNA, 30 bp, Artificial Sequence
  Synthetic Construct
ttttttttttagggcccagacactaacagc

SEQ ID NO: 269 - DNA, 19 bp, Artificial Sequence
  Synthetic Construct
ctaacagcacatctggaga

SEQ ID NO: 270 - DNA, 30 bp, Artificial Sequence
  Synthetic Construct
ttttttttttactaacagcacatctggaga

SEQ ID NO: 271 - DNA, 18 bp, Artificial Sequence
  Synthetic Construct
agaaagcaaattaaagat

SEQ ID NO: 272 - DNA, 28 bp, Artificial Sequence
  Synthetic Construct
ttttttttttagaaagcaaattaaagat

SEQ ID NO: 273 - DNA, 17 bp, Artificial Sequence
  Synthetic Construct
agcaaattaaagatcag

SEQ ID NO: 274 - DNA, 27 bp, Artificial Sequence
  Synthetic Construct
ttttttttttagcaaattaaagatcag

SEQ ID NO: 275 - DNA, 18 bp, Artificial Sequence
  Synthetic Construct
aaattaaagatcagtttc

SEQ ID NO: 276 - DNA, 28 bp, Artificial Sequence
  Synthetic Construct
ttttttttttaaattaaagatcagtttc

SEQ ID NO: 277 - DNA, 18 bp, Artificial Sequence
  Synthetic Construct
agatcagtttcctaattc

SEQ ID NO: 278 - DNA, 28 bp, Artificial Sequence
  Synthetic Construct
ttttttttttagatcagtttcctaattc

SEQ ID NO: 279 - DNA, 18 bp, Artificial Sequence
  Synthetic Construct
aagatcagtttcctaatt

SEQ ID NO: 280 - DNA, 28 bp, Artificial Sequence
  Synthetic Construct
ttttttttttaagatcagtttcctaatt

SEQ ID NO: 281 - DNA, 16 bp, Artificial Sequence
  Synthetic Construct
  misc_feature 14, 15, 16: n = inverted deoxythymidine
  misc_feature 15, 16: Can be present or absent
tacgccaccagctnnn

SEQ ID NO: 282 - DNA, 20 bp, Artificial Sequence
  Synthetic Construct
  misc_feature 18, 19, 20: n = inverted deoxythymidine
  misc_feature 19, 20: Can be present or absent
ttggagctggtggcgtannn

SEQ ID NO: 283 - DNA, 19 bp, Artificial Sequence
  Synthetic Construct
  misc_feature 17, 18, 19: n = inverted deoxythymidine
  misc_feature 18, 19: Can be present or absent
gctggtggcgtaggcannn

SEQ ID NO: 284 - DNA, 18 bp, Artificial Sequence
  Synthetic Construct
  misc_feature 16, 17, 18: n = inverted deoxythymidine
  misc_feature 17, 18: Can be present or absent
gctggtggcgtaggcnnn

SEQ ID NO: 285 - DNA, 19 bp, Artificial Sequence
  Synthetic Construct
  misc_feature 17, 18, 19: n = inverted deoxythymidine
  misc_feature 18, 19: Can be present or absent
ttggagctggtggcgtnnn

SEQ ID NO: 286 - DNA, 17 bp, Artificial Sequence
  Synthetic Construct
  misc_feature 15, 16, 17: n = inverted deoxythymidine
  misc_feature 16, 17: Can be present or absent
ctcttcttgtccagnnn

SEQ ID NO: 287 - DNA, 24 bp, Artificial Sequence
  Synthetic Construct
  misc_feature 22, 23, 24: n = inverted deoxythymidine
  misc_feature 23, 24: Can be present or absent
tcttcttgtccagctgtatccnnn

SEQ ID NO: 288 - DNA, 17 bp, Artificial Sequence
  Synthetic Construct
  misc_feature 15, 16, 17: n = inverted deoxythymidine
  misc_feature 16, 17: Can be present or absent
tcttgtccagctgtnnn

SEQ ID NO: 289 - DNA, 20 bp, Artificial Sequence
  Synthetic Construct
  misc_feature 18, 19, 20: n = inverted deoxythymidine
  misc_feature 19, 20: Can be present or absent
tcttgtccagctgtatcnnn

SEQ ID NO: 290 - DNA, 19 bp, Artificial Sequence
  Synthetic Construct
  misc_feature 17, 18, 19: n = inverted deoxythymidine
  misc_feature 18, 19: Can be present or absent
tcttcttgtccagctgnnn

SEQ ID NO: 291 - DNA, 21 bp, Artificial Sequence
  Synthetic Construct
  misc_feature 19, 20, 21: n = inverted deoxythymidine
  misc_feature 20, 21: Can be present or absent
ctgaaatcactgagcaggnnn

SEQ ID NO: 292 - DNA, 24 bp, Artificial Sequence
  Synthetic Construct
  misc_feature 22, 23, 24: n = inverted deoxythymidine
  misc_feature 23, 24: Can be present or absent
tctctgaaatcactgagcaggnnn

SEQ ID NO: 293 - DNA, 24 bp, Artificial Sequence
  Synthetic Construct
  misc_feature 22, 23, 24: n = inverted deoxythymidine
  misc_feature 23, 24: Can be present or absent
tctctgaaatcactgagcaggnnn

SEQ ID NO: 294 - DNA, 24 bp, Artificial Sequence
  Synthetic Construct
  misc_feature 22, 23, 24: n = inverted deoxythymidine
  misc_feature 23, 24: Can be present or absent
tctctgaaatcactgagcaggnnn

SEQ ID NO: 295 - DNA, 24 bp, Artificial Sequence
  Synthetic Construct
  misc_feature 22, 23, 24: n = inverted deoxythymidine
  misc_feature 23, 24: Can be present or absent
tctctgaattcactgagcaggnnn

SEQ ID NO: 296 - DNA, 18 bp, Artificial Sequence
  Synthetic Construct
  misc_feature 16, 17, 18: n = inverted deoxythymidine
  misc_feature 17, 18: Can be present or absent
caccatgatgtgcatnnn

SEQ ID NO: 297 - DNA, 19 bp, Artificial Sequence
  Synthetic Construct
  misc_feature 17, 18, 19: n = inverted deoxythymidine
  misc_feature 18, 19: Can be present or absent
ccaccatgatgtgcatnnn

SEQ ID NO: 298 - DNA, 18 bp, Artificial Sequence
  Synthetic Construct
  misc_feature 16, 17, 18: n = inverted deoxythymidine
  misc_feature 17, 18: Can be present or absent
caccatgatgtgcatnnn

SEQ ID NO: 299 - DNA, 21 bp, Artificial Sequence
  Synthetic Construct
  misc_feature 19, 20, 21: n = inverted deoxythymidine
  misc_feature 20, 21: Can be present or absent
ccaccatgatgtgcatcannn

SEQ ID NO: 300 - DNA, 14 bp, Artificial Sequence
  Synthetic Construct
  misc_feature 12, 13, 14: n = inverted deoxythymidine
  misc_feature 13, 14: Can be present or absent
catgatgtgcannn

SEQ ID NO: 301 - DNA, 19 bp, Artificial Sequence
  Synthetic Construct
  misc_feature 17, 18, 19: n = inverted deoxythymidine
  misc_feature 18, 19: Can be present or absent
gagatttcactgtagcnnn

SEQ ID NO: 302 - DNA, 19 bp, Artificial Sequence
  Synthetic Construct
  misc_feature 17, 18, 19: n = inverted deoxythymidine
  misc_feature 18, 19: Can be present or absent
gagatttcactgtagcnnn

SEQ ID NO: 303 - DNA, 19 bp, Artificial Sequence
  Synthetic Construct
  misc_feature 17, 18, 19: n = inverted deoxythymidine
  misc_feature 18, 19: Can be present or absent
gagatttcactgtagcnnn

SEQ ID NO: 304 - DNA, 19 bp, Artificial Sequence
  Synthetic Construct
  misc_feature 17, 18, 19: n = inverted deoxythymidine
  misc_feature 18, 19: Can be present or absent
gagatttcactgtagcnnn

SEQ ID NO: 305 - DNA, 19 bp, Artificial Sequence
  Synthetic Construct
  misc_feature 17, 18, 19: n = inverted deoxythymidine
  misc_feature 18, 19: Can be present or absent
gagatttcactgtagcnnn

SEQ ID NO: 306 - DNA, 21 bp, Artificial Sequence
  Synthetic Construct
  misc_feature 19, 20, 21: n = inverted deoxythymidine
  misc_feature 20, 21: Can be present or absent
cggagcccagcactttgannn

SEQ ID NO: 307 - DNA, 22 bp, Artificial Sequence
  Synthetic Construct
  misc_feature 20, 21, 22: n = inverted deoxythymidine
  misc_feature 21, 22: Can be present or absent
cgcaccggagcccagcactnnn

SEQ ID NO: 308 - DNA, 14 bp, Artificial Sequence
  Synthetic Construct
  misc_feature 12, 13, 14: n = inverted deoxythymidine
  misc_feature 13, 14: Can be present or absent
gagcccagcacnnn

SEQ ID NO: 309 - DNA, 21 bp, Artificial Sequence
  Synthetic Construct
  misc_feature 19, 20, 21: n = inverted deoxythymidine
  misc_feature 20, 21: Can be present or absent
cgcaccggagcccagcacnnn

SEQ ID NO: 310 - DNA, 24 bp, Artificial Sequence
  Synthetic Construct
  misc_feature 22, 23, 24: n = inverted deoxythymidine
  misc_feature 23, 24: Can be present or absent
cgcaccggagcccagcacttannn

SEQ ID NO: 311 - DNA, 28 bp, Artificial Sequence
  Synthetic Construct
  misc_feature 26, 27, 28: n = inverted deoxythymidine
  misc_feature 27, 28: Can be present or absent
cggagatgttgcttctcttaattccnnn

SEQ ID NO: 312 - DNA, 21 bp, Artificial Sequence
  Synthetic Construct
  misc_feature 19, 20, 21: n = inverted deoxythymidine
  misc_feature 20, 21: Can be present or absent
cggagatgttgcttctctnnn

SEQ ID NO: 313 - DNA, 21 bp, Artificial Sequence
  Synthetic Construct
  misc_feature 19, 20, 21: n = inverted deoxythymidine
  misc_feature 20, 21: Can be present or absent
gttgcttctcttaattccnnn

SEQ ID NO: 314 - DNA, 16 bp, Artificial Sequence
  Synthetic Construct
  misc_feature 14, 15, 16: n = inverted deoxythymidine
  misc_feature 15, 16: Can be present or absent
atgttgcttctctnnn

SEQ ID NO: 315 - DNA, 15 bp, Artificial Sequence
  Synthetic Construct
  misc_feature 13, 14, 15: n = inverted deoxythymidine
  misc_feature 14, 15: Can be present or absent
ttgcttctcttannn

SEQ ID NO: 316 - DNA, 19 bp, Artificial Sequence
  Synthetic Construct
  misc_feature 17, 18, 19: n = inverted deoxythymidine
  misc_feature 18, 19: Can be present or absent
catcacgcagctcatgnnn

SEQ ID NO: 317 - DNA, 21 bp, Artificial Sequence
  Synthetic Construct
  misc_feature 19, 20, 21: n = inverted deoxythymidine
  misc_feature 20, 21: Can be present or absent
tgcagctcatcacgcagcnnn

SEQ ID NO: 318 - DNA, 19 bp, Artificial Sequence
  Synthetic Construct
  misc_feature 17, 18, 19: n = inverted deoxythymidine
  misc_feature 18, 19: Can be present or absent
tcatcacgcagctcatnnn

SEQ ID NO: 319 - DNA, 15 bp, Artificial Sequence
  Synthetic Construct
  misc_feature 13, 14, 15: n = inverted deoxythymidine
  misc_feature 14, 15: Can be present or absent
tcatcacgcagcnnn

SEQ ID NO: 320 - DNA, 16 bp, Artificial Sequence
  Synthetic Construct
  misc_feature 14, 15, 16: n = inverted deoxythymidine
  misc_feature 15, 16: Can be present or absent
ctcatcacgcagcnnn

SEQ ID NO: 321 - DNA, 23 bp, Artificial Sequence
  Synthetic Construct
  misc_feature 21, 22, 23: n = inverted deoxythymidine
  misc_feature 22, 23: Can be present or absent
ccagcagtttggccagccctnnn

SEQ ID NO: 322 - DNA, 23 bp, Artificial Sequence
  Synthetic Construct
  misc_feature 21, 22, 23: n = inverted deoxythymidine
  misc_feature 22, 23: Can be present or absent
ccagcagtttggccagccctnnn

SEQ ID NO: 323 - DNA, 23 bp, Artificial Sequence
  Synthetic Construct
  misc_feature 21, 22, 23: n = inverted deoxythymidine
  misc_feature 22, 23: Can be present or absent
ccagcagtttggccagccctnnn

SEQ ID NO: 324 - DNA, 19 bp, Artificial Sequence
  Synthetic Construct
  misc_feature 17, 18, 19: n = inverted deoxythymidine
  misc_feature 18, 19: Can be present or absent
agcagtttggccagccnnn

SEQ ID NO: 325 - DNA, 23 bp, Artificial Sequence
  Synthetic Construct
  misc_feature 21, 22, 23: n = inverted deoxythymidine
  misc_feature 22, 23: Can be present or absent
ccagcagtttggccagccctnnn

SEQ ID NO: 326 - protein, 188 aa, Homo sapiens [sequence omitted]

SEQ ID NO: 327 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 328 - protein, 1068 aa, Homo sapiens
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 329 - protein, 766 aa, Homo sapiens [sequence omitted]

SEQ ID NO: 330 - protein, 1210 aa, Homo sapiens [sequence omitted]

SEQ ID NO: 331 - protein, 480 aa, Homo sapiens [sequence omitted]

SEQ ID NO: 332 - protein, 393 aa, Homo sapiens [sequence omitted]

SEQ ID NO: 333 - protein, 1055 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRNM

SEQ ID NO: 334 - protein, 1620 aa, Homo sapiens
MGAIGLLWLLPLLLSTAAVGSGMGTGQRAGSPAAGPPLQPREPLSYSRLQRKSLAVDFVVPSLFRVYARDLLLPPSSSELKAGRPEARGSLALDCAPLLRLLGPAPGVSWTAGSPAPAEARTLSRVLKGGSVRKLRRAKQLVLELGEEAILEGCVGPPGEAAVGLLQFNLSELFSWWIRQGEGRLRIRLMPEKKASEVGREGRLSAAIRASQPRLLFQIFGTGHSSLESPTNMPSPSPDYFTWNLTWIMKDSFPFLSHRSRYGLECSFDFPCELEYSPPLHDLRNQSWSWRRIPSEEASQMDLLDGPGAERSKEMPRGSFLLLNTSADSKHTILSPWMRSSSEHCTLAVSVHRHLQPSGRYIAQLLPHNEAAREILLMPTPGKHGWTVLQGRIGRPDNPFRVALEYISSGNRSLSAVDFFALKNCSEGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLPLSLILSVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 335 - protein, 2347 aa, Homo sapiens
MKNIYCLIPKLVNFATLGCLWISVVQCTVLNSCLKSCVTNLGQQLDLGTPHNLSEPCIQGCHFWNSVDQKNCALKCRESCEVGCSSAEGAYEEEVLENADLPTAPFASSIGSHNMTLRWKSANFSGVKYIIQWKYAQLLGSWTYTKTVSRPSYVVKPLHPFTEYIFRVVWIFTAQLQLYSPPSPSYRTHPHGVPETAPLIRNIESSSPDTVEVSWDPPQFPGGPILGYNLRLISKNQKLDAGTQRTSFQFYSTLPNTIYRFSIAAVNEVGEGPEAESSITTSSSAVQQEEQWLFLSRKTSLRKRSLKHLVDEAHCLRLDAIYHNITGISVDVHQQIVYFSEGTLIWAKKAANMSDVSDLRIFYRGSGLISSISIDWLYQRMYFIMDELVCVCDLENCSNIEEITPPSISAPQKIVADSYNGYVFYLLRDGIYRADLPVPSGRCAEAVRIVESCTLKDFAIKPQAKRIIYFNDTAQVFMSTFLDGSASHLILPRIPFADVKSFACENNDFLVTDGKVIFQQDALSFNEFIVGCDLSHIEEFGFGNLVIFGSSSQLHPLPGRPQELSVLFGSHQALVQWKPPALAIGANVILISDIIELFELGPSAWQNWTYEVKVSTQDPPEVTHIFLNISGTMLNVPELQSAMKYKVSVRASSPKRPGPWSEPSVGTTLVPASEPPFIMAVKEDGLWSKPLNSFGPGEFLSSDIGNVSDMDWYNNSLYYSDTKGDVFVWLLNGTDISENYHLPSIAGAGALAFEWLGHFLYWAGKTYVIQRQSVLTGHTDIVTHVKLLVNDMVVDSVGGYLYWTTLYSVESTRLNGESSLVLQTQPWFSGKKVIALTLDLSDGLLYWLVQDSQCIHLYTAVLRGQSTGDTTITEFAAWSTSEISQNALMYYSGRLFWINGFRIITTQEIGQKTSVSVLEPARFNQFTIIQTSLKPLPGNFSFTPKVIPDSVQESSFRIEGNASSFQILWNGPPAVDWGVVFYSVEFSAHSKFLASEQHSLPVFTVEGLEPYALFNLSVTPYTYWGKGPKTSLSLRAPETVPSAPENPRIFILPSGKCCNKNEVVVEFRWNKPKHENGVLTKFEIFYNISNQSITNKTCEDWIAVNVTPSVMSFQLEGMSPRCFIAFQVRAFTSKGPGPYADVVKSTTSEINPFPHLITLLGNKIVFLDMDQNQVVWTFSAERVISAVCYTADNEMGYYAEGDSLFLLHLHNRSSSELFQDSLVFDITVITIDWISRHLYFALKESQNGMQVFDVDLEHKVKYPREVKIHNRNSTIISFSVYPLLSRLYWTEVSNFGYQMFYYSIISHTLHRILQPTATNQQNKRNQCSCNVTEFELSGAMAIDTSNLEKPLIYFAKAQEIWAMDLEGCQCWRVITVPAMLAGKTLVSLTVDGDLIYWIITAKDSTQIYQAKKGNGAIVSQVKALRSRHILAYSSVMQPFPDKAFLSLASDTVEPTILNATNTSLTIRLPLAKTNLTWYGITSPTPTYLVYYAEVNDRKNSSDLKYRILEFQDSIALIEDLQPFSTYMIQIAVKNYYSDPLEHLPPGKEIWGKTKNGVPEAVQLINTTVRSDTSLIISWRESHKPNGPKESVRYQLAISHLALIPETPLRQSEFPNGRLTLLVTRLSGGNIYVLKVLACHSEEMWCTESHPVTVEMFNTPEKPYSLVPENTSLQFNWKAPLNVNLIRFWVELQKWKYNEFYHVKTSCSQGPAYVCNITNLQPYTSYNVRVVVVYKTGENSTSLPESFKTKAGVPNKPGIPKLLEGSKNSIQWEKAEDNGCRITYYILEIRKSTSNNLQNQNLRWKMTFNGSCSSVCTWKSKNLKGIFQFRVVAANNLGFGEYSGISENIILVGDDFWIPETSFILTIIVGIFLVVTIPLTFVWHRRLKNQKSAKEGVTVLINEDKELAELRGLAAGVGLANACYAIHTLPTQEEIENLPAFPREKLTLRLLLGSGAFGEVYEGTAVDILGVGSGEIKVAVKTLKKGSTDQEKIEFLKEAHLMSKFNHPNILKQLGVCLLNEPQYIILELMEGGDLLTYLRKARMATFYGPLLTLVDLVDLCVDISKGCVYLERMHFIHRDLAARNCLVSVKDYTSPRIVKIGDFGLARDIYKNDYYRKRGEGLLPVRWMAPESLMDGIFTTQSDVWSFGILIWEILTLGHQPYPAHSNLDVLNYVQTGGRLEPPRNCPDDLWNLMTQCWAQEPDQRPTFHRIQDQLQLFRNFFLNSIYKSRDEANNSGVINESFEGEDGDVICLNSDDIMPVALMETKNREGLNYMVLATECGQGEEKSEGPLGSQESESCGLRKEEKEPHADKDFCQEKQVAYCPSGKPEGLNYACLTHSGYGDGSD

SEQ ID NO: 336 - protein, 1072 aa, Homo sapiens
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 337 - protein, 760 aa, Homo sapiens
MKEAALICLAPSVPPILTVKSWDTMQLRAARSRCTNLLAASYIENQQHLQHLELRDLRGLGELRNLTIVKSGLRFVAPDAFHFTPRLSRLNLSFNALESLSWKTVQGLSLQELVLSGNPLHCSCALRWLQRWEEEGLGGVPEQKLQCHGQGPLAHMPNASCGVPTLKVQVPNASVDVGDDVLLRCQVEGRGLEQAGWILTELEQSATVMKSGGLPSLGLTLANVTSDLNRKNVTCWAENDVGRAEVSVQVNVSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPDTNSTSGDPVEKKDETPFGVSVAVGLAVFACLFLSTLLLVLNKCGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLG

SEQ ID NO: 338 - protein, 1390 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 339 - DNA, 859 bp, Homo sapiens
tgtgctcggagctcgattttcctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagagagatccgacaatacagattgaaaaaaatcagcaaagaagaaaagactcctggctgtgtgaaaattaaaaaatgcattataatgtaatctgggtgttgatgatgccttctatacattagttcgagaaattcgaaaacataaagaaaagatgagcaaagatggtaaaaagaagaaaaag

SEQ ID NO: 340 - DNA, 570 bp, Homo sapiens
atgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaa

SEQ ID NO: 341 - DNA, 3207 bp, Homo sapiens
atgcctccacgaccatcatcaggtgaactgtggggcatccacttgatgcccccaagaatcctagtagaatgtttactaccaaatggaatgatagtgactttagaatgcctccgtgaggctacattaataaccataaagcatgaactatttaaagaagcaagaaaataccccctccatcaacttcttcaagatgaatcttcttacattttcgtaagtgttactcaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataagatctatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtacagatgaagtttttagttgagcaaatgaggcgaccagatttcatggatgctctacagggctttctgtctcctctaaaccctgctcatcaactaggaaacctcaggcttgaagagtgtcgaattatgtcctctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactga

SEQ ID NO: 342 - DNA, 2301 bp, Homo sapiens
atggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactga

SEQ ID NO: 343 - DNA, 3633 bp, Homo sapiens
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatga

SEQ ID NO: 344 - DNA, 1443 bp, Homo sapiens
atgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctga

SEQ ID NO: 345 - DNA, 1182 bp, Homo sapiens
atgcccaagaagaagccgacgcccatccagctgaacccggcccccgacggctctgcagttaacgggaccagctctgcggagaccaacttggaggccttgcagaagaagctggaggagctagagcttgatgagcagcagcgaaagcgccttgaggcctttcttacccagaagcagaaggtgggagaactgaaggatgacgactttgagaagatcagtgagctgggggctggcaatggcggtgtggtgttcaaggtctcccacaagccttctggcctggtcatggccagaaagctaattcatctggagatcaaacccgcaatccggaaccagatcataagggagctgcaggttctgcatgagtgcaactctccgtacatcgtgggcttctatggtgcgttctacagcgatggcgagatcagtatctgcatggagcacatggatggaggttctctggatcaagtcctgaagaaagctggaagaattcctgaacaaattttaggaaaagttagcattgctgtaataaaaggcctgacatatctgagggagaagcacaagatcatgcacagagatgtcaagccctccaacatcctagtcaactcccgtggggagatcaagctctgtgactttggggtcagcgggcagctcatcgactccatggccaactccttcgtgggcacaaggtcctacatgtcgccagaaagactccaggggactcattactctgtgcagtcagacatctggagcatgggactgtctctggtagagatggcggttgggaggtatcccatccctcctccagatgccaaggagctggagctgatgtttgggtgccaggtggaaggagatgcggctgagaccccacccaggccaaggacccccgggaggccccttagctcatacggaatggacagccgacctcccatggcaatttttgagttgttggattacatagtcaacgagcctcctccaaaactgcccagtggagtgttcagtctggaatttcaagattttgtgaataaatgcttaataaaaaaccccgcagagagagcagatttgaagcaactcatggttcatgcttttatcaagagatctgatgctgaggaagtggattttgcaggttggctctgctccaccatcggccttaaccagcccagcacaccaacccatgctgctggcgtctaa

SEQ ID NO: 346 - DNA, 3168 bp, Homo sapiens
atggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggaatatgtga

SEQ ID NO: 347 - DNA, 4863 bp, Homo sapiens
atgggagccatcgggctcctgtggctcctgccgctgctgctttccacggcagctgtgggctccgggatggggaccggccagcgcgcgggctccccagctgcggggccgccgctgcagccccgggagccactcagctactcgcgcctgcagaggaagagtctggcagttgacttcgtggtgccctcgctcttccgtgtctacgcccgggacctactgctgccaccatcctcctcggagctgaaggctggcaggcccgaggcccgcggctcgctagctctggactgcgccccgctgctcaggttgctggggccggcgccgggggtctcctggaccgccggttcaccagccccggcagaggcccggacgctgtccagggtgctgaagggcggctccgtgcgcaagctccggcgtgccaagcagttggtgctggagctgggcgaggaggcgatcttggagggttgcgtcgggccccccggggaggcggctgtggggctgctccagttcaatctcagcgagctgttcagttggtggattcgccaaggcgaagggcgactgaggatccgcctgatgcccgagaagaaggcgtcggaagtgggcagagagggaaggctgtccgcggcaattcgcgcctcccagccccgccttctcttccagatcttcgggactggtcatagctccttggaatcaccaacaaacatgccttctccttctcctgattattttacatggaatctcacctggataatgaaagactccttccctttcctgtctcatcgcagccgatatggtctggagtgcagctttgacttcccctgtgagctggagtattcccctccactgcatgacctcaggaaccagagctggtcctggcgccgcatcccctccgaggaggcctcccagatggacttgctggatgggcctggggcagagcgttctaaggagatgcccagaggctcctttctccttctcaacacctcagctgactccaagcacaccatcctgagtccgtggatgaggagcagcagtgagcactgcacactggccgtctcggtgcacaggcacctgcagccctctggaaggtacattgcccagctgctgccccacaacgaggctgcaagagagatcctcctgatgcccactccagggaagcatggttggacagtgctccagggaagaatcgggcgtccagacaacccatttcgagtggccctggaatacatctccagtggaaaccgcagcttgtctgcagtggacttctttgccctgaagaactgcagtgaaggaacatccccaggctccaagatggccctgcagagctccttcacttgttggaatgggacagtcctccagcttgggcaggcctgtgacttccaccaggactgtgcccagggagaagatgagagccagatgtgccggaaactgcctgtgggtttttactgcaactttgaagatggcttctgtggctggacccaaggcacactgtcaccccacactcctcaatggcaggtcaggaccctaaaggatgcccggttccaggaccaccaagaccatgctctattgctcagtaccactgatgtccccgcttctgaaagtgctacagtgaccagtgctacgtttcctgcaccgatcaagagctctccatgtgagctccgaatgtcctggctcattcgtggagtcttgaggggaaacgtgtccttggtgctagtggagaacaaaaccgggaaggagcaaggcaggatggtctggcatgtcgccgcctatgaaggcttgagcctgtggcagtggatggtgttgcctctcctcgatgtgtctgacaggttctggctgcagatggtcgcatggtggggacaaggatccagagccatcgtggcttttgacaatatctccatcagcctggactgctacctcaccattagcggagaggacaagatcctgcagaatacagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctga

SEQ ID NO: 348 - DNA, 7044 bp, Homo sapiens
atgaagaacatttactgtcttattccgaagcttgtcaattttgcaactcttggctgcctatggatttctgtggtgcagtgtacagttttaaatagctgcctaaagtcgtgtgtaactaatctgggccagcagcttgaccttggcacaccacataatctgagtgaaccgtgtatccaaggatgtcacttttggaactctgtagatcagaaaaactgtgctttaaagtgtcgggagtcgtgtgaggttggctgtagcagcgcggaaggtgcatatgaagaggaagtactggaaaatgcagacctaccaactgctccctttgcttcttccattggaagccacaatatgacattacgatggaaatctgcaaacttctctggagtaaaatacatcattcagtggaaatatgcacaacttctgggaagctggacttatactaagactgtgtccagaccgtcctatgtggtcaagcccctgcaccccttcactgagtacattttccgagtggtttggatcttcacagcgcagctgcagctctactcccctccaagtcccagttacaggactcatcctcatggagttcctgaaactgcacctttgattaggaatattgagagctcaagtcccgacactgtggaagtcagctgggatccacctcaattcccaggtggacctattttgggttataacttaaggctgatcagcaaaaatcaaaaattagatgcagggacacagagaaccagtttccagttttactccactttaccaaatactatctacaggttttctattgcagcagtaaatgaagttggtgagggtccagaagcagaatctagtattaccacttcatcttcagcagttcaacaagaggaacagtggctctttttatccagaaaaacttctctaagaaagagatctttaaaacatttagtagatgaagcacattgccttcggttggatgctatataccataatattacaggaatatctgttgatgtccaccagcaaattgtttatttctctgaaggaactctcatatgggcgaagaaggctgccaacatgtctgatgtatctgacctgagaattttttacagaggttcaggattaatttcttctatctccatagattggctttatcaaagaatgtatttcatcatggatgaactggtatgtgtctgtgatttagagaactgctcaaacatcgaggaaattactccaccctctattagtgcacctcaaaaaattgtggctgattcatacaatgggtatgtcttttacctcctgagagatggcatttatagagcagaccttcctgtaccatctggccggtgtgcagaagctgtgcgtattgtggagagttgcacgttaaaggactttgcaatcaagccacaagccaagcgaatcatttacttcaatgacactgcccaagtcttcatgtcaacatttctggatggctctgcttcccatctcatcctacctcgcatcccctttgctgatgtgaaaagttttgcttgtgaaaacaatgactttcttgtcacagatggcaaggtcattttccaacaggatgctttgtcttttaatgaattcatcgtgggatgtgacctgagtcacatagaagaatttgggtttggtaacttggtcatctttggctcatcctcccagctgcaccctctgccaggccgcccgcaggagctttcggtgctgtttggctctcaccaggctcttgttcaatggaagcctcctgcccttgccataggagccaatgtcatcctgatcagtgatattattgaactctttgaattaggcccttctgcctggcagaactggacctatgaggtgaaagtatccacccaagaccctcctgaagtcactcatattttcttgaacataagtggaaccatgctgaatgtacctgagctgcagagtgctatgaaatacaaggtttctgtgagagcaagttctccaaagaggccaggcccctggtcagagccctcagtgggtactaccctggtgccagctagtgaaccaccatttatcatggctgtgaaagaagatgggctttggagtaaaccattaaatagctttggcccaggagagttcttatcctctgatataggaaatgtgtcagacatggattggtataacaacagcctctactacagtgacacgaaaggcgacgtttttgtgtggctgctgaatgggacggatatctcagagaattatcacctacccagcattgcaggagcaggggctttagcttttgagtggctgggtcactttctctactgggctggaaagacatatgtgatacaaaggcagtctgtgttgacgggacacacagacattgttacccacgtgaagctattggtgaatgacatggtggtggattcagttggtggatatctctactggaccacactctattcagtggaaagcaccagactaaatggggaaagttcccttgtactacagacacagccttggttttctgggaaaaaggtaattgctctaactttagacctcagtgatgggctcctgtattggttggttcaagacagtcaatgtattcacctgtacacagctgttcttcggggacagagcactggggataccaccatcacagaatttgcagcctggagtacttctgaaatttcccagaatgcactgatgtactatagtggtcggctgttctggatcaatggctttaggattatcacaactcaagaaataggtcagaaaaccagtgtctctgttttggaaccagccagatttaatcagttcacaattattcagacatcccttaagcccctgccagggaacttttcctttacccctaaggttattccagattctgttcaagagtcttcatttaggattgaaggaaatgcttcaagttttcaaatcctgtggaatggtccccctgcggtagactggggtgtagttttctacagtgtagaatttagtgctcattctaagttcttggctagtgaacaacactctttacctgtatttactgtggaaggactggaaccttatgccttatttaatctttctgtcactccttatacctactggggaaagggccccaaaacatctctgtcacttcgagcacctgaaacagttccatcagcaccagagaaccccagaatatttatattaccaagtggaaaatgctgcaacaagaatgaagttgtggtggaatttaggtggaacaaacctaagcatgaaaatggggtgttaacaaaatttgaaattttctacaatatatccaatcaaagtattacaaacaaaacatgtgaagactggattgctgtcaatgtcactccctcagtgatgtcttttcaacttgaaggcatgagtcccagatgctttattgccttccaggttagggcctttacatctaaggggccaggaccatatgctgacgttgtaaagtctacaacatcagaaatcaacccatttcctcacctcataactcttcttggtaacaagatagtttttttagatatggatcaaaatcaagttgtgtggacgttttcagcagaaagagttatcagtgccgtttgctacacagctgataatgagatgggatattatgctgaaggggactcactctttcttctgcacttgcacaatcgctctagctctgagcttttccaagattcactggtttttgatatcacagttattacaattgactggatttcaaggcacctctactttgcactgaaagaatcacaaaatggaatgcaagtatttgatgttgatcttgaacacaaggtgaaatatcccagagaggtgaagattcacaataggaattcaacaataatttctttttctgtatatcctcttttaagtcgcttgtattggacagaagtttccaattttggctaccagatgttctactacagtattatcagtcacaccttgcaccgaattctgcaacccacagctacaaaccaacaaaacaaaaggaatcaatgttcttgtaatgtgactgaatttgagttaagtggagcaatggctattgatacctctaacctagagaaaccattgatatactttgccaaagcacaagagatctgggcaatggatctggaaggctgtcagtgttggagagttatcacagtacctgctatgctcgcaggaaaaacccttgttagcttaactgtggatggagatcttatatactggatcatcacagcaaaggacagcacacagatttatcaggcaaagaaaggaaatggggccatcgtttcccaggtgaaggccctaaggagtaggcatatcttggcttacagttcagttatgcagccttttccagataaagcgtttctgtctctagcttcagacactgtggaaccaactatacttaatgccactaacactagcctcacaatcagattacctctggccaagacaaacctcacatggtatggcatcaccagccctactccaacatacctggtttattatgcagaagttaatgacaggaaaaacagctctgacttgaaatatagaattctggaatttcaggacagtatagctcttattgaagatttacaaccattttcaacatacatgatacagatagctgtaaaaaattattattcagatcctttggaacatttaccaccaggaaaagagatttggggaaaaactaaaaatggagtaccagaggcagtgcagctcattaatacaactgtgcggtcagacaccagcctcattatatcttggagagaatctcacaagccaaatggacctaaagaatcagtccgttatcagttggcaatctcacacctggccctaattcctgaaactcctctaagacaaagtgaatttccaaatggaaggctcactctccttgttactagactgtctggtggaaatatttatgtgttaaaggttcttgcctgccactctgaggaaatgtggtgtacagagagtcatcctgtcactgtggaaatgtttaacacaccagagaaaccttattccttggttccagagaacactagtttgcaatttaattggaaggctccattgaatgttaacctcatcagattttgggttgagctacagaagtggaaatacaatgagttttaccatgttaaaacttcatgcagccaaggtcctgcttatgtctgtaatatcacaaatctacaaccttatacttcatataatgtcagagtagtggtggtttataagacgggagaaaatagcacctcacttccagaaagctttaagacaaaagctggagtcccaaataaaccaggcattcccaaattactagaagggagtaaaaattcaatacagtgggagaaagctgaagataatggatgtagaattacatactatatccttgagataagaaagagcacttcaaataatttacagaaccagaatttaaggtggaagatgacatttaatggatcctgcagtagtgtttgcacatggaagtccaaaaacctgaaaggaatatttcagttcagagtagtagctgcaaataatctagggtttggtgaatatagtggaatcagtgagaatattatattagttggagatgatttttggataccagaaacaagtttcatacttactattatagttggaatatttctggttgttacaatcccactgacctttgtctggcatagaagattaaagaatcaaaaaagtgccaaggaaggggtgacagtgcttataaacgaagacaaagagttggctgagctgcgaggtctggcagccggagtaggcctggctaatgcctgctatgcaatacatactcttccaacccaagaggagattgaaaatcttcctgccttccctcgggaaaaactgactctgcgtctcttgctgggaagtggagcctttggagaagtgtatgaaggaacagcagtggacatcttaggagttggaagtggagaaatcaaagtagcagtgaagactttgaagaagggttccacagaccaggagaagattgaattcctgaaggaggcacatctgatgagcaaatttaatcatcccaacattctgaagcagcttggagtttgtctgctgaatgaaccccaatacattatcctggaactgatggagggaggagaccttcttacttatttgcgtaaagcccggatggcaacgttttatggtcctttactcaccttggttgaccttgtagacctgtgtgtagatatttcaaaaggctgtgtctacttggaacggatgcatttcattcacagggatctggcagctagaaattgccttgtttccgtgaaagactataccagtccacggatagtgaagattggagactttggactcgccagagacatctataaaaatgattactatagaaagagaggggaaggcctgctcccagttcggtggatggctccagaaagtttgatggatggaatcttcactactcaatctgatgtatggtcttttggaattctgatttgggagattttaactcttggtcatcagccttatccagctcattccaaccttgatgtgttaaactatgtgcaaacaggagggagactggagccaccaagaaattgtcctgatgatctgtggaatttaatgacccagtgctgggctcaagaacccgaccaaagacctacttttcatagaattcaggaccaacttcagttattcagaaattttttcttaaatagcatttataagtccagagatgaagcaaacaacagtggagtcataaatgaaagctttgaaggtgaagatggcgatgtgatttgtttgaattcagatgacattatgccagttgctttaatggaaacgaagaaccgagaagggttaaactatatggtacttgctacagaatgtggccaaggtgaagaaaagtctgagggtcctctaggctcccaggaatctgaatcttgtggtctgaggaaagaagagaaggaaccacatgcagacaaagatttctgccaagaaaaacaagtggcttactgcccttctggcaagcctgaaggcctgaactatgcctgtctcactcacagtggatatggagatgggtctgattaa

SEQ ID NO: 349 - DNA, 3219 bp, Homo sapiens
atggcgaaggcgacgtccggtgccgcggggctgcgtctgctgttgctgctgctgctgccgctgctaggcaaagtggcattgggcctctacttctcgagggatgcttactgggagaagctgtatgtggaccaggcagccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctag

SEQ ID NO: 350 - DNA, 2283 bp, Homo sapiens
atgaaggaggccgccctcatctgcctggcaccctctgtacccccgatcttgacggtgaagtcctgggacaccatgcagttgcgggctgctagatctcggtgcacaaacttgttggcagcaagctacatcgagaaccagcagcatctgcagcatctggagctccgtgatctgaggggcctgggggagctgagaaacctcaccatcgtgaagagtggtctccgtttcgtggcgccagatgccttccatttcactcctcggctcagtcgcctgaatctctccttcaacgctctggagtctctctcctggaaaactgtgcagggcctctccttacaggaactggtcctgtcggggaaccctctgcactgttcttgtgccctgcgctggctacagcgctgggaggaggagggactgggcggagtgcctgaacagaagctgcagtgtcatgggcaagggcccctggcccacatgcccaatgccagctgtggtgtgcccacgctgaaggtccaggtgcccaatgcctcggtggatgtgggggacgacgtgctgctgcggtgccaggtggaggggcggggcctggagcaggccggctggatcctcacagagctggagcagtcagccacggtgatgaaatctgggggtctgccatccctggggctgaccctggccaatgtcaccagtgacctcaacaggaagaacgtgacgtgctgggcagagaacgatgtgggccgggcagaggtctctgttcaggtcaacgtctccttcccggccagtgtgcagctgcacacggcggtggagatgcaccactggtgcatccccttctctgtggatgggcagccggcaccgtctctgcgctggctcttcaatggctccgtgctcaatgagaccagcttcatcttcactgagttcctggagccggcagccaatgagaccgtgcggcacgggtgtctgcgcctcaaccagcccacccacgtcaacaacggcaactacacgctgctggctgccaaccccttcggccaggcctccgcctccatcatggctgccttcatggacaaccctttcgagttcaaccccgaggaccccatccctgacactaacagcacatctggagacccggtggagaagaaggacgaaacaccttttggggtctcggtggctgtgggcctggccgtctttgcctgcctcttcctttctacgctgctccttgtgctcaacaaatgtggacggagaaacaagtttgggatcaaccgcccggctgtgctggctccagaggatgggctggccatgtccctgcatttcatgacattgggtggcagctccctgtcccccaccgagggcaaaggctctgggctccaaggccacatcatcgagaacccacaatacttcagtgatgcctgtgttcaccacatcaagcgccgggacatcgtgctcaagtgggagctgggggagggcgcctttgggaaggtcttccttgctgagtgccacaacctcctgcctgagcaggacaagatgctggtggctgtcaaggcactgaaggaggcgtccgagagtgctcggcaggacttccagcgtgaggctgagctgctcaccatgctgcagcaccagcacatcgtgcgcttcttcggcgtctgcaccgagggccgccccctgctcatggtctttgagtatatgcggcacggggacctcaaccgcttcctccgatcccatggacctgatgccaagctgctggctggtggggaggatgtggctccaggccccctgggtctggggcagctgctggccgtggctagccaggtcgctgcggggatggtgtacctggcgggtctgcattttgtgcaccgggacctggccacacgcaactgtctagtgggccagggactggtggtcaagattggtgattttggcatgagcagggatatctacagcaccgactattaccgtgtgggaggccgcaccatgctgcccattcgctggatgccgcccgagagcatcctgtaccgtaagttcaccaccgagagcgacgtgtggagcttcggcgtggtgctctgggagatcttcacctacggcaagcagccctggtaccagctctccaacacggaggcaatcgactgcatcacgcagggacgtgagttggagcggccacgtgcctgcccaccagaggtctacgccatcatgcggggctgctggcagcgggagccccagcaacgccacagcatcaaggatgtgcacgcccggctgcaagccctggcccaggcacctcctgtctacctggatgtcctgggctag

SEQ ID NO: 351 - DNA, 4227 bp, Homo sapiens
atgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtacttggtggaaagaacctctcaacattgtcagttttctattttgctttgccagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatcatag

SEQ ID NO: 352 - DNA, 25 bp, Artificial Sequence
  Synthetic Construct
tttttttttttgagatgcatgatga

SEQ ID NO: 353 - DNA, 25 bp, Artificial Sequence
  Synthetic Construct
ttttttttttgagatgcatgatgag

SEQ ID NO: 354 - DNA, 25 bp, Artificial Sequence
  Synthetic Construct
ttttttttatgagatgcatgatgag

SEQ ID NO: 355 - DNA, 25 bp, Artificial Sequence
  Synthetic Construct
tttttttttttgagctgcatgatga

SEQ ID NO: 356 - DNA, 25 bp, Artificial Sequence
  Synthetic Construct
ttttttttcatgagatgcatgatga

SEQ ID NO: 357 - DNA, 25 bp, Artificial Sequence
  Synthetic Construct
tatggagcaaaactactgtagagcc

SEQ ID NO: 358 - DNA, 24 bp, Artificial Sequence
  Synthetic Construct
ccagctacatcacacaccttgact

SEQ ID NO: 359 - DNA, 26 bp, Artificial Sequence
  Synthetic Construct
taataccaaaagttaccaaaactgca

SEQ ID NO: 360 - DNA, 23 bp, Artificial Sequence
  Synthetic Construct
caatctctgaagatcatgtggcc

SEQ ID NO: 361 - DNA, 19 bp, Artificial Sequence
  Synthetic Construct
caagtggcacagtggtggc

SEQ ID NO: 362 - DNA, 23 bp, Artificial Sequence
  Synthetic Construct
taactggaggagggaaagacaga

SEQ ID NO: 363 - DNA, 22 bp, Artificial Sequence
  Synthetic Construct
agttggggttgtagtcggtcat

SEQ ID NO: 364 - DNA, 19 bp, Artificial Sequence
  Synthetic Construct
gaagcctccctggatctcc

SEQ ID NO: 365 - DNA, 14 bp, Artificial Sequence
  Synthetic Construct
  misc_feature 12, 13, 14: n = inverted deoxythymidine
  misc_feature 13, 14: Can be present or absent
catcacgcagcnnn

SEQ ID NO: 366 - DNA, 13 bp, Artificial Sequence
  Synthetic Construct
  misc_feature 11, 12, 13: n = inverted deoxythymidine
  misc_feature 12, 13: Can be present or absent
catcacgcagnnn

SEQ ID NO: 367 - DNA, 13 bp, Artificial Sequence
  Synthetic Construct
  misc_feature 11, 12, 13: n = inverted deoxythymidine
  misc_feature 12, 13: Can be present or absent
atcacgcagcnnn

SEQ ID NO: 368 - DNA, 14 bp, Artificial Sequence
  Synthetic Construct
  misc_feature 12, 13, 14: n = inverted deoxythymidine
  misc_feature 13, 14: Can be present or absent
catcacgcagcnnn

SEQ ID NO: 369 - DNA, 13 bp, Artificial Sequence
  Synthetic Construct
  misc_feature 11, 12, 13: n = inverted deoxythymidine
  misc_feature 12, 13: Can be present or absent
catcacgcagnnn

SEQ ID NO: 370 - DNA, 14 bp, Artificial Sequence
  Synthetic Construct
tgtagggaagtaca

SEQ ID NO: 371 - DNA, 27 bp, Artificial Sequence
  Synthetic Construct
ttttttttttttttgtagggaagtaca

SEQ ID NO: 372 - DNA, 15 bp, Artificial Sequence
  Synthetic Construct
tctgtagggaagtac

SEQ ID NO: 373 - DNA, 26 bp, Artificial Sequence
  Synthetic Construct
ttttttttttttctgtagggaagtac

SEQ ID NO: 374 - DNA, 18 bp, Artificial Sequence
  Synthetic Construct
gtctgtagggaagtacat

SEQ ID NO: 375 - DNA, 25 bp, Artificial Sequence
  Synthetic Construct
tttttttgtctgtagggaagtacat

SEQ ID NO: 376 - DNA, 17 bp, Artificial Sequence
  Synthetic Construct
ccgcacgtctgtaggga

SEQ ID NO: 377 - DNA, 24 bp, Artificial Sequence
  Synthetic Construct
tttttttccgcacgtctgtaggga

SEQ ID NO: 378 - DNA, 17 bp, Artificial Sequence
  Synthetic Construct
acgtctgtagggaagta

SEQ ID NO: 379 - DNA, 25 bp, Artificial Sequence
  Synthetic Construct
ttttttttacgtctgtagggaagta

SEQ ID NO: 380 - DNA, 21 bp, Artificial Sequence
  Synthetic Construct
gagggtctgacgggtagagtg

SEQ ID NO: 381 - DNA, 21 bp, Artificial Sequence
  Synthetic Construct
tggccgccaggtcttgatgta

SEQ ID NO: 382 - DNA, 17 bp, Artificial Sequence
  Synthetic Construct
  misc_feature 15, 16, 17: n = inverted deoxythymidine
  misc_feature 16, 17: Can be present or absent
tgtactcccctacannn

SEQ ID NO: 383 - DNA, 16 bp, Artificial Sequence
  Synthetic Construct
  misc_feature 14, 15, 16: n = inverted deoxythymidine
  misc_feature 15, 16: Can be present or absent
gatgtactcccctnnn

SEQ ID NO: 384 - DNA, 17 bp, Artificial Sequence
  Synthetic Construct
  misc_feature 15, 16, 17: n = inverted deoxythymidine
  misc_feature 16, 17: Can be present or absent
atgtactcccctacnnn

SEQ ID NO: 385 - DNA, 16 bp, Artificial Sequence
  Synthetic Construct
  misc_feature 14, 15, 16: n = inverted deoxythymidine
  misc_feature 15, 16: Can be present or absent
gtactcccctacannn

SEQ ID NO: 386 - DNA, 19 bp, Artificial Sequence
  Synthetic Construct
  misc_feature 17, 18, 19: n = inverted deoxythymidine
  misc_feature 18, 19: Can be present or absent
gatgtactcccctacannn

SEQ ID NO: 387 - DNA, 16 bp, Artificial Sequence
  Synthetic Construct
ttacccagaatcagaa

SEQ ID NO: 388 - DNA, 25 bp, Artificial Sequence
  Synthetic Construct
tttttaaatttacccagaatcagaa

SEQ ID NO: 389 - DNA, 16 bp, Artificial Sequence
  Synthetic Construct
ccagaatcagaaggtg

SEQ ID NO: 390 - DNA, 25 bp, Artificial Sequence
  Synthetic Construct
tttttaaatccagaatcagaaggtg

SEQ ID NO: 391 - DNA, 16 bp, Artificial Sequence
  Synthetic Construct
ttcttacccagaatca

SEQ ID NO: 392 - DNA, 25 bp, Artificial Sequence
  Synthetic Construct
tttttaaatttcttacccagaatca

SEQ ID NO: 393 - DNA, 18 bp, Artificial Sequence
  Synthetic Construct
cctttcttacccagaatc

SEQ ID NO: 394 - DNA, 27 bp, Artificial Sequence
  Synthetic Construct
tttttaaatcctttcttacccagaatc

SEQ ID NO: 395 - DNA, 16 bp, Artificial Sequence
  Synthetic Construct
cagaatcagaaggtgg

SEQ ID NO: 396 - DNA, 25 bp, Artificial Sequence
  Synthetic Construct
tttttaaatcagaatcagaaggtgg

SEQ ID NO: 397 - DNA, 20 bp, Artificial Sequence
  Synthetic Construct
cttgatgagcagcagcgaaa

SEQ ID NO: 398 - DNA, 22 bp, Artificial Sequence
  Synthetic Construct
ccttcagttctcccaccttctg

SEQ ID NO: 399 - DNA, 19 bp, Artificial Sequence
  Synthetic Construct
  misc_feature 17, 18, 19: n = inverted deoxythymidine
  misc_feature 18, 19: Can be present or absent
tctgcttctgggtaagnnn

SEQ ID NO: 400 - DNA, 21 bp, Artificial Sequence
  Synthetic Construct
  misc_feature 19, 20, 21: n = inverted deoxythymidine
  misc_feature 20, 21: Can be present or absent
ttctgcttctgggtaagannn

SEQ ID NO: 401 - DNA, 20 bp, Artificial Sequence
  Synthetic Construct
  misc_feature 18, 19, 20: n = inverted deoxythymidine
  misc_feature 19, 20: Can be present or absent
caccttctgcttctgggnnn

SEQ ID NO: 402 - DNA, 17 bp, Artificial Sequence
  Synthetic Construct
  misc_feature 15, 16, 17: n = inverted deoxythymidine
  misc_feature 16, 17: Can be present or absent
tctgcttctgggtannn

SEQ ID NO: 403 - DNA, 25 bp, Artificial Sequence
  Synthetic Construct
  misc_feature 23, 24, 25: n = inverted deoxythymidine
  misc_feature 24, 25: Can be present or absent
caccttctgcttctgggtaagannn

SEQ ID NO: 404 - DNA, 16 bp, Artificial Sequence
  Synthetic Construct
atacgtgatgtcttac

SEQ ID NO: 405 - DNA, 25 bp, Artificial Sequence
  Synthetic Construct
tttttttttatacgtgatgtcttac

SEQ ID NO: 406 - DNA, 16 bp, Artificial Sequence
  Synthetic Construct
acgtgatggcttacgt

SEQ ID NO: 407 - DNA, 27 bp, Artificial Sequence
  Synthetic Construct
tttttttttttacgtgatggcttacgt

SEQ ID NO: 408 - DNA, 17 bp, Artificial Sequence
  Synthetic Construct
aagcatacgtgatggct

SEQ ID NO: 409 - DNA, 22 bp, Artificial Sequence
  Synthetic Construct
tttttaagcatacgtgatggct

SEQ ID NO: 410 - DNA, 16 bp, Artificial Sequence
  Synthetic Construct
gcatacgtgatggctt

SEQ ID NO: 411 - DNA, 23 bp, Artificial Sequence
  Synthetic Construct
tttttttgcatacgtgatggctt

SEQ ID NO: 412 - DNA, 17 bp, Artificial Sequence
  Synthetic Construct
gcatacgtgatggctta

SEQ ID NO: 413 - DNA, 24 bp, Artificial Sequence
  Synthetic Construct
tttttttgcatacgtgatggctta

SEQ ID NO: 414 - DNA, 21 bp, Artificial Sequence
  Synthetic Construct
atggctgtggtttgtgatggt

SEQ ID NO: 415 - DNA, 22 bp, Artificial Sequence
  Synthetic Construct
acaccagccatcacgtaagaca

SEQ ID NO: 416 - DNA, 15 bp, Artificial Sequence
  Synthetic Construct
  misc_feature 13, 14, 15: n = inverted deoxythymidine
  misc_feature 14, 15: Can be present or absent
ggctgcctcctgnnn

SEQ ID NO: 417 - DNA, 16 bp, Artificial Sequence
  Synthetic Construct
  misc_feature 14, 15, 16: n = inverted deoxythymidine
  misc_feature 15, 16: Can be present or absent
cggctgcctcctgnnn

SEQ ID NO: 418 - DNA, 16 bp, Artificial Sequence
  Synthetic Construct
  misc_feature 14, 15, 16: n = inverted deoxythymidine
  misc_feature 15, 16: Can be present or absent
cggctgcctcctgnnn

SEQ ID NO: 419 - DNA, 17 bp, Artificial Sequence
  Synthetic Construct
  misc_feature 15, 16, 17: n = inverted deoxythymidine
  misc_feature 16, 17: Can be present or absent
tcggctgcctcctgnnn

SEQ ID NO: 420 - DNA, 16 bp, Artificial Sequence
  Synthetic Construct
  misc_feature 14, 15, 16: n = inverted deoxythymidine
  misc_feature 15, 16: Can be present or absent
tcggctgcctcctnnn

SEQ ID NO: 421 - DNA, 13 bp, Artificial Sequence
  Synthetic Construct
atggccatcttgg

SEQ ID NO: 422 - DNA, 22 bp, Artificial Sequence
  Synthetic Construct
tttttttttatggccatcttgg

SEQ ID NO: 423 - DNA, 12 bp, Artificial Sequence
  Synthetic Construct
ggccatcttgga

SEQ ID NO: 424 - DNA, 23 bp, Artificial Sequence
  Synthetic Construct
ttttttttttaggccatcttgga

SEQ ID NO: 425 - DNA, 13 bp, Artificial Sequence
  Synthetic Construct
gatggccatcttg

SEQ ID NO: 426 - DNA, 21 bp, Artificial Sequence
  Synthetic Construct
tttttttagatggccatcttg

SEQ ID NO: 427 - DNA, 14 bp, Artificial Sequence
  Synthetic Construct
tgatggccatcttg

SEQ ID NO: 428 - DNA, 21 bp, Artificial Sequence
  Synthetic Construct
ttttttttgatggccatcttg

SEQ ID NO: 429 - DNA, 12 bp, Artificial Sequence
  Synthetic Construct
tggccatcttgg

SEQ ID NO: 430 - DNA, 22 bp, Artificial Sequence
  Synthetic Construct
tttttttttttggccatcttgg

SEQ ID NO: 431 - DNA, 11 bp, Artificial Sequence
  Synthetic Construct
gtgatggccgg

SEQ ID NO: 432 - DNA, 22 bp, Artificial Sequence
  Synthetic Construct
tttttttttttgtgatggccgg

SEQ ID NO: 433 - DNA, 12 bp, Artificial Sequence
  Synthetic Construct
tgatggccggcg

SEQ ID NO: 434 - DNA, 24 bp, Artificial Sequence
  Synthetic Construct
tttttttttttttgatggccggcg

SEQ ID NO: 435 - DNA, 14 bp, Artificial Sequence
  Synthetic Construct
gtgatggccggcgt

SEQ ID NO: 436 - DNA, 25 bp, Artificial Sequence
  Synthetic Construct
tttttttttttgtgatggccggcgt

SEQ ID NO: 437 - DNA, 12 bp, Artificial Sequence
  Synthetic Construct
gatggccggcgt

SEQ ID NO: 438 - DNA, 25 bp, Artificial Sequence
  Synthetic Construct
tttttttttttttgatggccggcgt

SEQ ID NO: 439 - DNA, 13 bp, Artificial Sequence
  Synthetic Construct
gatggcccgcgtg

SEQ ID NO: 440 - DNA, 26 bp, Artificial Sequence
  Synthetic Construct
tttttttttttttgatggcccgcgtg

SEQ ID NO: 441 - DNA, 14 bp, Artificial Sequence
  Synthetic Construct
aacccccatcacgt

SEQ ID NO: 442 - DNA, 25 bp, Artificial Sequence
  Synthetic Construct
tttttttttttaacccccatcacgt

SEQ ID NO: 443 - DNA, 16 bp, Artificial Sequence
  Synthetic Construct
gacaacccccatcacg

SEQ ID NO: 444 - DNA, 24 bp, Artificial Sequence
  Synthetic Construct
ttttttttgacaacccccatcacg

SEQ ID NO: 445 - DNA, 18 bp, Artificial Sequence
  Synthetic Construct
cgtggacaacccccatca

SEQ ID NO: 446 - DNA, 22 bp, Artificial Sequence
  Synthetic Construct
ttttcgtggacaacccccatca

SEQ ID NO: 447 - DNA, 12 bp, Artificial Sequence
  Synthetic Construct
cccatcacgtgt

SEQ ID NO: 448 - DNA, 24 bp, Artificial Sequence
  Synthetic Construct
ttttttttttttcccatcacgtgt

SEQ ID NO: 449 - DNA, 17 bp, Artificial Sequence
  Synthetic Construct
tggacaacccccatcac

SEQ ID NO: 450 - DNA, 24 bp, Artificial Sequence
  Synthetic Construct
ttttttttggacaacccccatcac

SEQ ID NO: 451 - DNA, 14 bp, Artificial Sequence
  Synthetic Construct
gccagcgtggacgg

SEQ ID NO: 452 - DNA, 25 bp, Artificial Sequence
  Synthetic Construct
tttttttttttgccagcgtggacgg

SEQ ID NO: 453 - DNA, 14 bp, Artificial Sequence
  Synthetic Construct
cgtggacggtaacc

SEQ ID NO: 454 - DNA, 25 bp, Artificial Sequence
  Synthetic Construct
tttttttttttcgtggacggtaacc

SEQ ID NO: 455 - DNA, 13 bp, Artificial Sequence
  Synthetic Construct
gacggtaaccccc

SEQ ID NO: 456 - DNA, 24 bp, Artificial Sequence
  Synthetic Construct
tttttttttttgacggtaaccccc

SEQ ID NO: 457 - DNA, 14 bp, Artificial Sequence
  Synthetic Construct
ccagcgtggacggt

SEQ ID NO: 458 - DNA, 24 bp, Artificial Sequence
  Synthetic Construct
ttttttttttccagcgtggacggt

SEQ ID NO: 459 - DNA, 16 bp, Artificial Sequence
  Synthetic Construct
gccagcgtggacggta

SEQ ID NO: 460 - DNA, 23 bp, Artificial Sequence
  Synthetic Construct
tttttttgccagcgtggacggta

SEQ ID NO: 461 - DNA, 14 bp, Artificial Sequence
  Synthetic Construct
ccaggaggctgccg

SEQ ID NO: 462 - DNA, 26 bp, Artificial Sequence
  Synthetic Construct
tttttttttttaccaggaggctgccg

SEQ ID NO: 463 - DNA, 14 bp, Artificial Sequence
  Synthetic Construct
caggaggctgccga

SEQ ID NO: 464 - DNA, 26 bp, Artificial Sequence
  Synthetic Construct
tttttttttttacaggaggctgccga

SEQ ID NO: 465 - DNA, 14 bp, Artificial Sequence
  Synthetic Construct
tccaggaggctgcc

SEQ ID NO: 466 - DNA, 26 bp, Artificial Sequence
  Synthetic Construct
tttttttttttatccaggaggctgcc

SEQ ID NO: 467 - DNA, 13 bp, Artificial Sequence
  Synthetic Construct
ccaggaggctgcc

SEQ ID NO: 468 - DNA, 25 bp, Artificial Sequence
  Synthetic Construct
tttttttttttaccaggaggctgcc

SEQ ID NO: 469 - DNA, 12 bp, Artificial Sequence
  Synthetic Construct
caggaggctgcc

SEQ ID NO: 470 - DNA, 24 bp, Artificial Sequence
  Synthetic Construct
tttttttttttacaggaggctgcc

SEQ ID NO: 471 - DNA, 13 bp, Artificial Sequence
  Synthetic Construct
ccaggagggagcc

SEQ ID NO: 472 - DNA, 25 bp, Artificial Sequence
  Synthetic Construct
tttttttttttaccaggagggagcc

SEQ ID NO: 473 - DNA, 14 bp, Artificial Sequence
  Synthetic Construct
ccaggagggagccg

SEQ ID NO: 474 - DNA, 26 bp, Artificial Sequence
  Synthetic Construct
tttttttttttaccaggagggagccg

SEQ ID NO: 475 - DNA, 14 bp, Artificial Sequence
  Synthetic Construct
tccaggagggagcc

SEQ ID NO: 476 - DNA, 26 bp, Artificial Sequence
  Synthetic Construct
tttttttttttatccaggagggagcc

SEQ ID NO: 477 - DNA, 13 bp, Artificial Sequence
  Synthetic Construct
caggagggagccg

SEQ ID NO: 478 - DNA, 25 bp, Artificial Sequence
  Synthetic Construct
tttttttttttacaggagggagccg

SEQ ID NO: 479 - DNA, 14 bp, Artificial Sequence
  Synthetic Construct
caggagggagccga

SEQ ID NO: 480 - DNA, 26 bp, Artificial Sequence
  Synthetic Construct
tttttttttttacaggagggagccga

SEQ ID NO: 481 - DNA, 21 bp, Artificial Sequence
  Synthetic Construct
cttcgtatctctcaagaggat

SEQ ID NO: 482 - DNA, 30 bp, Artificial Sequence
  Synthetic Construct
tttttttttcttcgtatctctcaagaggat

SEQ ID NO: 483 - DNA, 21 bp, Artificial Sequence
  Synthetic Construct
gtatctctcaagaggatccaa

SEQ ID NO: 484 - DNA, 30 bp, Artificial Sequence
  Synthetic Construct
tttttttttgtatctctcaagaggatccaa

SEQ ID NO: 485 - DNA, 17 bp, Artificial Sequence
  Synthetic Construct
ttcgtatctctcaagag

SEQ ID NO: 486 - DNA, 26 bp, Artificial Sequence
  Synthetic Construct
tttttttttttcgtatctctcaagag

SEQ ID NO: 487 - DNA, 15 bp, Artificial Sequence
  Synthetic Construct
tcaagaggatccaaa

SEQ ID NO: 488 - DNA, 24 bp, Artificial Sequence
  Synthetic Construct
ttttttttttcaagaggatccaaa

SEQ ID NO: 489 - DNA, 12 bp, Artificial Sequence
  Synthetic Construct
tctctcaagagg

SEQ ID NO: 490 - DNA, 21 bp, Artificial Sequence
  Synthetic Construct
ttttttttttctctcaagagg

SEQ ID NO: 491 - DNA, 19 bp, Artificial Sequence
  Synthetic Construct
gttaaaaaggaggatccaa

SEQ ID NO: 492 - DNA, 28 bp, Artificial Sequence
  Synthetic Construct
tttttttttgttaaaaaggaggatccaa

SEQ ID NO: 493 - DNA, 20 bp, Artificial Sequence
  Synthetic Construct
acaagagttaaaaaggagga

SEQ ID NO: 494 - DNA, 28 bp, Artificial Sequence
  Synthetic Construct
ttttttttacaagagttaaaaaggagga

SEQ ID NO: 495 - DNA, 20 bp, Artificial Sequence
  Synthetic Construct
aagagttaaaaaggaggatc

SEQ ID NO: 496 - DNA, 28 bp, Artificial Sequence
  Synthetic Construct
ttattattaagagttaaaaaggaggatc

SEQ ID NO: 497 - DNA, 17 bp, Artificial Sequence
  Synthetic Construct
aaaaggaggatccaaag

SEQ ID NO: 498 - DNA, 25 bp, Artificial Sequence
  Synthetic Construct
ttttttttaaaaggaggatccaaag

SEQ ID NO: 499 - DNA, 17 bp, Artificial Sequence
  Synthetic Construct
aaggaggatccaaagtg

SEQ ID NO: 500 - DNA, 25 bp, Artificial Sequence
  Synthetic Construct
ttttttttaaggaggatccaaagtg

SEQ ID NO: 501 - DNA, 17 bp, Artificial Sequence
  Synthetic Construct
aaacaggaggatccaaa

SEQ ID NO: 502 - DNA, 25 bp, Artificial Sequence
  Synthetic Construct
ttttttttaaacaggaggatccaaa

SEQ ID NO: 503 - DNA, 18 bp, Artificial Sequence
  Synthetic Construct
aagtgcacaaacaggagg

SEQ ID NO: 504 - DNA, 26 bp, Artificial Sequence
  Synthetic Construct
tttttattaagtgcacaaacaggagg

SEQ ID NO: 505 - DNA, 19 bp, Artificial Sequence
  Synthetic Construct
gtgcacaaacaggaggatc

SEQ ID NO: 506 - DNA, 28 bp, Artificial Sequence
  Synthetic Construct
tattattatgtgcacaaacaggaggatc

SEQ ID NO: 507 - DNA, 15 bp, Artificial Sequence
  Synthetic Construct
cacaaacaggaggat

SEQ ID NO: 508 - DNA, 23 bp, Artificial Sequence
  Synthetic Construct
tattttttcacaaacaggaggat

SEQ ID NO: 509 - DNA, 16 bp, Artificial Sequence
  Synthetic Construct
aacaggaggatccaaa

SEQ ID NO: 510 - DNA, 24 bp, Artificial Sequence
  Synthetic Construct
ttttatttaacaggaggatccaaa

SEQ ID NO: 511 - DNA, 18 bp, Artificial Sequence
  Synthetic Construct
ccacactgacgtgcctct

SEQ ID NO: 512 - DNA, 23 bp, Artificial Sequence
  Synthetic Construct
gcacacgtaggggttgtccaaga

SEQ ID NO: 513 - DNA, 18 bp, Artificial Sequence
  Synthetic Construct
ccacactgacgtgcctct

SEQ ID NO: 514 - DNA, 19 bp, Artificial Sequence
  Synthetic Construct
gtacacgctggccacgccg

SEQ ID NO: 515 - DNA, 18 bp, Artificial Sequence
  Synthetic Construct
ccacactgacgtgcctct

SEQ ID NO: 516 - DNA, 17 bp, Artificial Sequence
  Synthetic Construct
caggcggcacacgtgat

SEQ ID NO: 517 - DNA, 18 bp, Artificial Sequence
  Synthetic Construct
ccacactgacgtgcctct

SEQ ID NO: 518 - DNA, 22 bp, Artificial Sequence
  Synthetic Construct
aggcggcacacgtgcgggttac

SEQ ID NO: 519 - DNA, 22 bp, Artificial Sequence
  Synthetic Construct
aaggagttagcagcatgtcagc

SEQ ID NO: 520 - DNA, 24 bp, Artificial Sequence
  Synthetic Construct
aacttcagactttacacaacctgc

SEQ ID NO: 521 - DNA, 21 bp, Artificial Sequence
  Synthetic Construct
attgattctgatgacaccgga